(19) United States
(12) Patent Application Publication
(10) Pub. No.: US 2025/0297255 A1
(43) Pub. Date: Sep. 25, 2025

(54) SYSTEMS TARGETING TMPRSS4 AND SLC34A2

(71) Applicants: Arsenal Biosciences, Inc., South San Francisco, CA (US); Juno Therapeutics, Inc., Seattle, WA (US)

(72) Inventors: Jasper Williams, San Francisco, CA (US); Michelle Nguyen, San Francisco, CA (US); Rona Harari Steinfeld, Sunnyvale, CA (US); Jianying Liu, Albany, CA (US); Matthew Drever, Concord, CA (US); Samuel A. Williams, Burlingame, CA (US); Stephanie Elise Busch, Seattle, WA (US); Pallavur V. Sivakumar, Seattle, WA (US); Gabriela Hernandez-Hoyos, Seattle, WA (US); Brian Andrew Fox, Seattle, WA (US); Cédric Cleyrat, Seattle, WA (US); Christopher Mark Hill, Seattle, WA (US); Lore K. Florin, Redwood City, CA (US); Nels B. Hamacher, Mountlake Terrace, WA (US); Craig Darwin Ostrander, Bothell, WA (US); Sergio L. Lacayo, Castro Valley, CA (US)

(21) Appl. No.: 19/085,815

(22) Filed: Mar. 20, 2025

Related U.S. Application Data
(60) Provisional application No. 63/567,860, filed on Mar. 20, 2024.
(60) Provisional application No. 63/771,558, filed on Mar. 13, 2025.

(51) Int. Cl. C12N 15/113; A61K 40/10; A61P 35/00; C07K 16/00; C12N 5/07

(52) U.S. Cl. CPC C12N 15/113; A61K 40/10; A61P 35/00; C07K 16/00; C12N 5/06; C07K 2317/24; C07K 2317/565; C12N 2510/00; C12N 2730/10011

(57) ABSTRACT

Provided herein are polypeptides, nucleic acids, and cells comprising antigen-binding domains that specifically bind to Solute Carrier Family 34 Member 2 (SLC34A2) or Transmembrane protease, serine 4 (TMPRSS4), and methods of use thereof. Also provided are polypeptides, systems, nucleic acids, and cells comprising priming receptors comprising an antigen-binding domain that specifically binds Solute Carrier Family 34 Member 2 (SLC34A2) and chimeric antigen receptors (CAR) comprising an antigen-binding domain that specifically binds to Transmembrane protease, serine 4 (TMPRSS4).

### CROSS-REFERENCE TO RELATED APPLICATIONS

This application claims the benefit of U.S. Provisional Application No. 63/567,860, filed Mar. 20, 2024, and U.S. Provisional Application No. 63/771,558, filed Mar. 13, 2025, both which are hereby incorporated in their entirety by reference.

### SEQUENCE LISTING

The instant application contains a Sequence Listing XML which has been submitted electronically and is hereby incorporated by reference in its entirety. Said XML copy, created on Mar. 13, 2025, is named ANB-501US_SL.xml, and is 1,393,781 bytes in size.

### BACKGROUND

TMPRSS4, is a 48 kDa transmembrane glycoprotein that belongs to the serine protease family of proteins, a promoter of cancer cell invasion. The canonical isoform encodes a type II single pass transmembrane protein with a 384 amino acid extracellular C-terminal domain. An autocatalytic event has been reported to induce self-cleavage between amino acids 204 and 205, resulting in a 150 amino acid extracellular domain. Elevated expression of TMPRSS4 correlates with poor prognosis in colorectal cancer, gastric cancer, prostate cancer, non-small cell lung cancer, and other cancers. Therefore, cancer immunotherapy using T cells redirected with TMPRSS4 receptor may show antitumor functions.

Cancer is a disease characterized by uncontrollable growth of cells. Many approaches to treating cancer have been tried, including drugs and radiation therapies. Recent cancer treatments have sought to use the body's own immune cells to attack cancer cells. One promising approach uses T cells that are taken from a patient and genetically engineered to produce chimeric antigen receptors, or CARs, receptor proteins that give the T cells a new ability to target a specific protein. The receptors are chimeric because they combine antigen-binding and T-cell activating functions into a single receptor. Immunotherapy using CAR-T cells is promising because the modified T cells have the potential to recognize cancer cells in order to more effectively target and destroy them.

However, there remain some concerns and limitations to CAR T cell-based immunotherapy. Some CAR T cells may engage with normal cells expressing low levels of target antigens, leading to leading to on-target, off tumor toxicity. Thus, additional therapies that reduce off-tumor toxicity remain desirable.

### SUMMARY

In one aspect, provided herein are systems comprising:
a. a first chimeric polypeptide comprising a priming receptor comprising a first antigen-binding domain that specifically binds Solute Carrier Family 34 Member 2 (SLC34A2) (SEQ ID NO: 962); and
b. a second chimeric polypeptide comprising a chimeric antigen receptor (CAR) comprising a second antigen-binding domain that specifically binds to Transmembrane protease, serine 4 (TMPRSS4) (SEQ ID NO: 960).

In some embodiments, the first antigen-binding domain comprises a first variable heavy (VH) chain sequence comprising three heavy chain CDR sequences, CDR-H1, CDR-H2, and CDR-H3, of the VH sequences set forth in SEQ ID NOs: 1001, 1009, or 1015, and a first variable light (VL) chain sequence comprising three light chain CDR sequences, CDR-L1, CDR-L2, and CDR-L3, of the VL sequences set forth in SEQ ID NOs: 1005, 1013, 1125, or 1019, optionally wherein:
a. CDR-H1 comprises the sequence set forth in SEQ ID NO: 1002, CDR-H2 comprises the sequence set forth in SEQ ID NO: 1003, CDR-H3 comprises the sequence set forth in SEQ ID NO: 1004, CDR-L1 comprises the sequence set forth in SEQ ID NO: 1006, CDR-L2 comprises the sequence set forth in SEQ ID NO: 1007, and CDR-L3 comprises the sequence set forth in SEQ ID NO: 1008; or
b. CDR-H1 comprises the sequence set forth in SEQ ID NO: 1010, CDR-H2 comprises the sequence set forth in SEQ ID NO: 1011, CDR-H3 comprises the sequence set forth in SEQ ID NO: 1012, CDR-L1 comprises the sequence set forth in SEQ ID NO: 1006, CDR-L2 comprises the sequence set forth in SEQ ID NO: 1007, and CDR-L3 comprises the sequence set forth in SEQ ID NO: 1014; or
c. CDR-H1 comprises the sequence set forth in SEQ ID NO: 1016, CDR-H2 comprises the sequence set forth in SEQ ID NO: 1017, CDR-H3 comprises the sequence set forth in SEQ ID NO: 1018, CDR-L1 comprises the sequence set forth in SEQ ID NO: 1020, CDR-L2 comprises the sequence set forth in SEQ ID NO: 1021, and CDR-L3 comprises the sequence set forth in SEQ ID NO: 1022.

In some embodiments, the first VH chain sequence comprises the sequence set forth in SEQ ID NO: 1001, 1009, or 1015.

In some embodiments, the first VL chain sequence comprises the sequence set forth in SEQ ID NO: 1005, 1013, 1125, or 1019.

In some embodiments, wherein the first antigen-binding domain comprises the sequence set forth in SEQ ID NO: 1107, 1108, or 1109.

In some embodiments, the second antigen-binding domain comprises a second variable heavy (VH) chain sequence comprising three heavy chain CDR sequences, CDR-H1, CDR-H2, and CDR-H3, of the VH sequences set forth in SEQ ID NOs: 319 or 326, and a second variable light (VL) chain sequence comprising three light chain CDR sequences, CDR-L1, CDR-L2, and CDR-L3, of the VL sequence set forth in SEQ ID NOs: 320 or 327, optionally wherein:
a. CDR-H1 comprises the sequence set forth in SEQ ID NO: 321, CDR-H2 comprises the sequence set forth in SEQ ID NO: 322, CDR-H3 comprises the sequence set forth in SEQ ID NO: 323, CDR-L1 comprises the sequence set forth in SEQ ID NO: 324, CDR-L2 comprises the sequence set forth in SEQ ID NO: 325, and CDR-L3 comprises the sequence set forth in SEQ ID NO: 16; and
b. CDR-H1 comprises the sequence set forth in SEQ ID NO: 193, CDR-H2 comprises the sequence set forth in SEQ ID NO: 80, CDR-H3 comprises the sequence set forth in SEQ ID NO: 328, CDR-L1 comprises the sequence set forth in SEQ ID NO: 329, CDR-L2 comprises the sequence set forth in SEQ ID NO: 330, and CDR-L3 comprises the sequence set forth in SEQ ID NO: 331.

In some embodiments, the second VH comprises the sequence as set forth in SEQ ID NOs: 319 or 326.

In some embodiments, the second VL comprises the sequence set forth in SEQ ID NOs: 320 or 327.

In some embodiments, the second antigen binding domain comprises the sequence set forth in SEQ ID NO: 551 or 552.

In some embodiments,
a. the first antigen-binding domain comprises the first variable heavy (VH) chain sequence comprising three heavy chain CDR sequences, CDR-H1, CDR-H2, and CDR-H3, of the VH sequence set forth in SEQ ID NO: 1009, and the first variable light (VL) chain sequence comprising three light chain CDR sequences, CDR-L1, CDR-L2, and CDR-L3, of the VL sequence set forth in SEQ ID NO: 1013 and the second antigen-binding domain comprises the first variable heavy (VH) chain sequence comprising three heavy chain CDR sequences, CDR-H1, CDR-H2, and CDR-H3, of the VH sequence set forth in SEQ ID NO: 326, and the first variable light (VL) chain sequence comprising three light chain CDR sequences, CDR-L1, CDR-L2, and CDR-L3, of the VL sequence set forth in SEQ ID NO: 327;
b. the first antigen-binding domain comprises the first variable heavy (VH) chain sequence comprising three heavy chain CDR sequences, CDR-H1, CDR-H2, and CDR-H3, of the VH sequence set forth in SEQ ID NO: 1001, and the first variable light (VL) chain sequence comprising three light chain CDR sequences, CDR-L1, CDR-L2, and CDR-L3, of the VL sequence set forth in SEQ ID NO: 1005 and
the second antigen-binding domain comprises the first variable heavy (VH) chain sequence comprising three heavy chain CDR sequences, CDR-H1, CDR-H2, and CDR-H3, of the VH sequence set forth in SEQ ID NO: 326, and the first variable light (VL) chain sequence comprising three light chain CDR sequences, CDR-L1, CDR-L2, and CDR-L3, of the VL sequence set forth in SEQ ID NO: 327;
c. the first antigen-binding domain comprises the first variable heavy (VH) chain sequence comprising three heavy chain CDR sequences, CDR-H1, CDR-H2, and CDR-H3, of the VH sequence set forth in SEQ ID NO: 1009, and the first variable light (VL) chain sequence comprising three light chain CDR sequences, CDR-L1, CDR-L2, and CDR-L3, of the VL sequence set forth in SEQ ID NO: 1013 and
the second antigen-binding domain comprises the first variable heavy (VH) chain sequence comprising three heavy chain CDR sequences, CDR-H1, CDR-H2, and CDR-H3, of the VH sequence set forth in SEQ ID NO: 319, and the first variable light (VL) chain sequence comprising three light chain CDR sequences, CDR-L1, CDR-L2, and CDR-L3, of the VL sequence set forth in SEQ ID NO: 320;
d. the first antigen-binding domain comprises the first variable heavy (VH) chain sequence comprising three heavy chain CDR sequences, CDR-H1, CDR-H2, and CDR-H3, of the VH sequence set forth in SEQ ID NO: 1015, and the first variable light (VL) chain sequence comprising three light chain CDR sequences, CDR-L1, CDR-L2, and CDR-L3, of the VL sequence set forth in SEQ ID NO: 1019 or 1125 and
the second antigen-binding domain comprises the first variable heavy (VH) chain sequence comprising three heavy chain CDR sequences, CDR-H1, CDR-H2, and CDR-H3, of the VH sequence set forth in SEQ ID NO: 319, and the first variable light (VL) chain sequence comprising three light chain CDR sequences, CDR-L1, CDR-L2, and CDR-L3, of the VL sequence set forth in SEQ ID NO: 320; or
e. the first antigen-binding domain comprises the first variable heavy (VH) chain sequence comprising three heavy chain CDR sequences, CDR-H1, CDR-H2, and CDR-H3, of the VH sequence set forth in SEQ ID NO: 1009, and the first variable light (VL) chain sequence comprising three light chain CDR sequences, CDR-L1, CDR-L2, and CDR-L3, of the VL sequence set forth in SEQ ID NO: 1013 and
the second antigen-binding domain comprises the first variable heavy (VH) chain sequence comprising three heavy chain CDR sequences, CDR-H1, CDR-H2, and CDR-H3, of the VH sequence set forth in SEQ ID NO: 326, and the first variable light (VL) chain sequence comprising three light chain CDR sequences, CDR-L1, CDR-L2, and CDR-L3, of the VL sequence set forth in SEQ ID NO: 327.

In some embodiments,
a. the first antigen-binding domain comprises a CDR-H1 comprises the sequence set forth in SEQ ID NO: 1010, CDR-H2 comprises the sequence set forth in SEQ ID NO: 1011, CDR-H3 comprises the sequence set forth in SEQ ID NO: 1012, CDR-L1 comprises the sequence set forth in SEQ ID NO: 1006, CDR-L2 comprises the sequence set forth in SEQ ID NO: 1007, and CDR-L3 comprises the sequence set forth in SEQ ID NO: 1014 and
the second antigen-binding domain comprises a CDR-H1 comprises the sequence set forth in SEQ ID NO: 193, CDR-H2 comprises the sequence set forth in SEQ ID NO: 80, CDR-H3 comprises the sequence set forth in SEQ ID NO: 328, CDR-L1 comprises the sequence set forth in SEQ ID NO: 329, CDR-L2 comprises the sequence set forth in SEQ ID NO: 330, and CDR-L3 comprises the sequence set forth in SEQ ID NO: 331;
b. the first antigen-binding domain comprises a CDR-H1 comprises the sequence set forth in SEQ ID NO: 1002, CDR-H2 comprises the sequence set forth in SEQ ID NO: 1003, CDR-H3 comprises the sequence set forth in SEQ ID NO: 1004, CDR-L1 comprises the sequence set forth in SEQ ID NO: 1006, CDR-L2 comprises the sequence set forth in SEQ ID NO: 1007, and CDR-L3 comprises the sequence set forth in SEQ ID NO: 1008 and
the second antigen-binding domain comprises a CDR-H1 comprises the sequence set forth in SEQ ID NO: 193, CDR-H2 comprises the sequence set forth in SEQ ID NO: 80, CDR-H3 comprises the sequence set forth in SEQ ID NO: 328, CDR-L1 comprises the sequence set forth in SEQ ID NO: 329, CDR-L2 comprises the sequence set forth in SEQ ID NO: 330, and CDR-L3 comprises the sequence set forth in SEQ ID NO: 331;
c. the first antigen-binding domain comprises a CDR-H1 comprises the sequence set forth in SEQ ID NO: 1010, CDR-H2 comprises the sequence set forth in SEQ ID NO: 1011, CDR-H3 comprises the sequence set forth in SEQ ID NO: 1012, CDR-L1 comprises the sequence set forth in SEQ ID NO: 1006, CDR-L2 comprises the sequence set forth in SEQ ID NO: 1007, and CDR-L3 comprises the sequence set forth in SEQ ID NO: 1014 and
the second antigen-binding domain comprises a CDR-H1 comprises the sequence set forth in SEQ ID NO: 321, CDR-H2 comprises the sequence set forth in SEQ ID NO: 322, CDR-H3 comprises the sequence set forth in SEQ ID NO: 323, CDR-L1 comprises the sequence set forth in SEQ ID NO: 324, CDR-L2 comprises the sequence set forth in SEQ ID NO: 325, and CDR-L3 comprises the sequence set forth in SEQ ID NO: 16;
d. the first antigen-binding domain comprises a CDR-H1 comprises the sequence set forth in SEQ ID NO: 1016, CDR-H2 comprises the sequence set forth in SEQ ID NO: 1017, CDR-H3 comprises the sequence set forth in SEQ ID NO: 1018, CDR-L1 comprises the sequence set forth in SEQ ID NO: 1020, CDR-L2 comprises the sequence set forth in SEQ ID NO: 1021, and CDR-L3 comprises the sequence set forth in SEQ ID NO: 1022 and
the second antigen-binding domain comprises a CDR-H1 comprises the sequence set forth in SEQ ID NO: 321, CDR-H2 comprises the sequence set forth in SEQ ID NO: 322, CDR-H3 comprises the sequence set forth in SEQ ID NO: 323, CDR-L1 comprises the sequence set forth in SEQ ID NO: 324, CDR-L2 comprises the sequence set forth in SEQ ID NO: 325, and CDR-L3 comprises the sequence set forth in SEQ ID NO: 16; or
e. the first antigen-binding domain comprises a CDR-H1 comprises the sequence set forth in SEQ ID NO: 1010, CDR-H2 comprises the sequence set forth in SEQ ID NO: 1011, CDR-H3 comprises the sequence set forth in SEQ ID NO: 1012, CDR-L1 comprises the sequence set forth in SEQ ID NO: 1006, CDR-L2 comprises the sequence set forth in SEQ ID NO: 1007, and CDR-L3 comprises the sequence set forth in SEQ ID NO: 1014 and
the second antigen-binding domain comprises a CDR-H1 comprises the sequence set forth in SEQ ID NO: 193, CDR-H2 comprises the sequence set forth in SEQ ID NO: 80, CDR-H3 comprises the sequence set forth in SEQ ID NO: 328, CDR-L1 comprises the sequence set forth in SEQ ID NO: 329, CDR-L2 comprises the sequence set forth in SEQ ID NO: 330, and CDR-L3 comprises the sequence set forth in SEQ ID NO: 331.

In some embodiments, at least one or more nucleic acids comprising a nucleic acid sequence at least 15 nucleotides in length complementary to a portion thereof of:
a. a nucleic acid encoding human Fas Cell Surface Death Receptor (FAS) comprising the sequence set forth in SEQ ID NO: 964; and/or
b. a nucleic encoding human Transforming Growth factor (TGF)-β Receptor 2 (TGFBR2) comprising the sequence set forth in SEQ ID NO: 965; and/or
c. a nucleic acid encoding Phosphatase Non-Receptor Type 2 (PTPN2) comprising the sequence set forth in SEQ ID NO: 966.

In some embodiments, the at least one or more nucleic acids comprises a nucleic acid comprising the sequence as set forth in SEQ ID NO: 967.

In some embodiments, the at least one or more nucleic acids comprises a nucleic acid comprising the sequence as set forth in SEQ ID NOs: 969 and/or 970.

In some embodiments, the at least one or more nucleic acids comprises a nucleic acid comprising the sequence as set forth in SEQ ID NO: 968.

In some embodiments, the at least one or more nucleic acids comprises a first nucleic acid comprising the sequence as set forth in SEQ ID NO: 967, a second nucleic acid comprising the sequence as set forth in SEQ ID NOs: 969 and/or 970, and a third nucleic acid comprising the sequence as set forth in SEQ ID NO: 968.

In some embodiments, the at least one or more nucleic acids comprises a first nucleic acid comprising the sequence as set forth in SEQ ID NO: 967, a second nucleic acid comprising the sequence as set forth in SEQ ID NO: 969, and a third nucleic acid comprising the sequence as set forth in SEQ ID NO: 970, and a fourth nucleic acid comprising the sequence as set forth in SEQ ID NO: 968.

In some embodiments,
a. the nucleic acid is capable of reducing expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid;
b. the nucleic acid is capable of reducing expression of TGFBR2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid; and/or
c. the nucleic acid is capable of reducing expression of PTPN2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In one aspect, provided herein are systems comprising:
a. a first chimeric polypeptide comprising a priming receptor comprising a first antigen-binding domain that specifically binds Solute Carrier Family 34 Member 2 (SLC34A2) (SEQ ID NO: 962);
b. a second chimeric polypeptide comprising a chimeric antigen receptor (CAR) comprising a second antigen-binding domain that specifically binds to Transmembrane protease, serine 4 (TMPRSS4) (SEQ ID NO: 960); and
c. at least one or more nucleic acids comprising a nucleic acid sequence at least 15 nucleotides in length complementary to a portion thereof of:
   i. a nucleic acid encoding human Fas Cell Surface Death Receptor (FAS) comprising the sequence set forth in SEQ ID NO: 964; and/or
   ii. a nucleic acid encoding human Transforming Growth factor (TGF)-β Receptor 2 (TGFBR2) comprising the sequence set forth in SEQ ID NO: 965; and/or
   iii. a nucleic acid encoding Phosphatase Non-Receptor Type 2 (PTPN2) comprising the sequence set forth in SEQ ID NO: 966.

In some embodiments, the first antigen-binding domain comprises a first variable heavy (VH) chain sequence comprising three heavy chain CDR sequences, CDR-H1, CDR-H2, and CDR-H3, of the VH sequences set forth in SEQ ID NOs: 1001, 1009, or 1015, and a first variable light (VL) chain sequence comprising three light chain CDR sequences, CDR-L1, CDR-L2, and CDR-L3, of the VL sequences set forth in SEQ ID NOs: 1005, 1013, 1125, or 1019, optionally wherein:
a. CDR-H1 comprises the sequence set forth in SEQ ID NO: 1002, CDR-H2 comprises the sequence set forth in SEQ ID NO: 1003, CDR-H3 comprises the sequence set forth in SEQ ID NO: 1004, CDR-L1 comprises the sequence set forth in SEQ ID NO: 1006, CDR-L2 comprises the sequence set forth in SEQ ID NO: 1007, and CDR-L3 comprises the sequence set forth in SEQ ID NO: 1008; or
b. CDR-H1 comprises the sequence set forth in SEQ ID NO: 1010, CDR-H2 comprises the sequence set forth in SEQ ID NO: 1011, CDR-H3 comprises the sequence set forth in SEQ ID NO: 1012, CDR-L1 comprises the sequence set forth in SEQ ID NO: 1006, CDR-L2 comprises the sequence set forth in SEQ ID NO: 1007, and CDR-L3 comprises the sequence set forth in SEQ ID NO: 1014; or
c. CDR-H1 comprises the sequence set forth in SEQ ID NO: 1016, CDR-H2 comprises the sequence set forth in SEQ ID NO: 1017, CDR-H3 comprises the sequence set forth in SEQ ID NO: 1018, CDR-L1 comprises the sequence set forth in SEQ ID NO: 1020, CDR-L2 comprises the sequence set forth in SEQ ID NO: 1021, and CDR-L3 comprises the sequence set forth in SEQ ID NO: 1022.

In some embodiments, the first VH chain sequence comprises the sequence set forth in SEQ ID NOs: 1001, 1009, or 1015.

In some embodiments, the first VL chain sequence comprises the sequence set forth in SEQ ID NOs: 1005, 1013, 1125, or 1019.

In some embodiments, the first antigen-binding domain comprises the sequence set forth in SEQ ID NOs: 1107, 1108, or 1109.

In some embodiments, the second antigen-binding domain comprises a second variable heavy (VH) chain sequence comprising three heavy chain CDR sequences, CDR-H1, CDR-H2, and CDR-H3, of the VH sequences set forth in SEQ ID NOs: 319 or 326, and a second variable light (VL) chain sequence comprising three light chain CDR sequences, CDR-L1, CDR-L2, and CDR-L3, of the VL sequence set forth in SEQ ID NOs: 320 or 327, optionally wherein:
a. CDR-H1 comprises the sequence set forth in SEQ ID NO: 321, CDR-H2 comprises the sequence set forth in SEQ ID NO: 322, CDR-H3 comprises the sequence set forth in SEQ ID NO: 323, CDR-L1 comprises the sequence set forth in SEQ ID NO: 324, CDR-L2 comprises the sequence set forth in SEQ ID NO: 325, and CDR-L3 comprises the sequence set forth in SEQ ID NO: 16; and
b. CDR-H1 comprises the sequence set forth in SEQ ID NO: 193, CDR-H2 comprises the sequence set forth in SEQ ID NO: 80, CDR-H3 comprises the sequence set forth in SEQ ID NO: 328, CDR-L1 comprises the sequence set forth in SEQ ID NO: 329, CDR-L2 comprises the sequence set forth in SEQ ID NO: 330, and CDR-L3 comprises the sequence set forth in SEQ ID NO: 331.

In some embodiments, the second VH comprises the sequence as set forth in SEQ ID NOs: 319 or 326.

In some embodiments, the second VL comprises the sequence set forth in SEQ ID NOS: 320 or 327.

In some embodiments, the second antigen binding domain comprises the sequence set forth in SEQ ID NOs: 551 or 552.

In some embodiments, the priming receptor comprises, from N-terminus to C-terminus,
a. the first antigen-binding domain;
b. a first transmembrane domain comprising one or more ligand-inducible proteolytic cleavage sites; and
c. an intracellular domain comprising a human or humanized transcriptional effector, wherein binding of the first antigen-binding domain to human SCL34A2 results in cleavage at the one or more ligand-inducible proteolytic cleavage sites.

In some embodiments, the priming receptor comprises a first hinge domain positioned between the first antigen-binding domain and the first transmembrane domain.

In some embodiments, the first hinge domain comprises a CD8α or truncated CD8α hinge domain.

In some embodiments, the first hinge comprises the sequence as set forth in SEQ ID NO: 827.

In some embodiments, the first transmembrane domain comprises a Notch1 transmembrane domain.

In some embodiments, the transmembrane domain comprises the sequence as set forth in SEQ ID NO: 828.

In some embodiments, the intracellular domain comprises an HNF1α/p65 domain or a Gal4/VP64 domain.

In some embodiments, the intracellular domain comprises the sequence as set forth in SEQ ID NO: 830, 831, or 832.

In some embodiments, the priming receptor comprises a stop-transfer-sequence or juxtamembrane domain between the first transmembrane domain and the intracellular domain.

In some embodiments, the stop-transfer-sequence or juxtamembrane domain comprises the sequence as set forth in SEQ ID NO: 829.

In some embodiments, the priming receptor comprises a sequence as set forth in SEQ ID NO: 1158, 1160, or 1162.

In some embodiments, the CAR comprises, from N-terminus to C-terminus,
a. a second antigen-binding domain;
b. a second transmembrane domain;
c. an intracellular co-stimulatory domain; and
d. an intracellular activation domain.

In some embodiments, the CAR comprises a second hinge domain.

In some embodiments, the second hinge domain comprises a CD8α or truncated CD8α hinge domain.

In some embodiments, the second hinge domain comprises a sequence as set forth in SEQ ID NO: 821.

In some embodiments, the second transmembrane domain comprises a CD8α transmembrane domain.

In some embodiments, the second transmembrane domain comprises a sequence as set forth in SEQ ID NO: 822.

In some embodiments, the intracellular co-stimulatory domain comprises a 4-1BB domain.

In some embodiments, the intracellular co-stimulatory domain comprises a sequence as set forth in SEQ ID NO: 823.

In some embodiments, the intracellular activation domain comprises a CD35 domain.

In some embodiments, the intracellular activation domain comprises a sequence as set forth in SEQ ID NO: 824.

In some embodiments, the CAR comprises a sequence as set forth in SEQ ID NOs: 1164 or 1166.

In some embodiments, the priming receptor and the CAR are capable of binding to a same target cell if the target cell expresses SLC34A2 and TMPRSS4.

In some embodiments, the at least one or more nucleic acids comprising a nucleic acid sequence at least 15 nucleotides in length complementary a portion thereof of human FAS, human TGFBR2 and/or human PTPN2 are at least 16, 17, 18, 19, 20, 21, or 22 nucleotides in length.

In some embodiments, the at least one or more nucleic acid sequences are a short hairpin RNA (shRNA), a small interfering RNA (siRNA), a double stranded RNA (dsRNA), or an antisense oligonucleotide.

In some embodiments, the at least one or more nucleic acid sequences are shRNA.

In some embodiments, the at least one or more nucleic acids comprises a sequence selected from the group consisting of the sequences set forth in SEQ ID NOs: 967-970.

In some embodiments, the nucleic acid sequence complementary to a nucleic acid encoding human FAS comprises a sequence as set forth in SEQ ID NO: 967.

In some embodiments, the nucleic acid reduces expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the nucleic acid sequence complementary to a nucleic acid encoding human TGFBR2 comprises a sequence as set forth in SEQ ID NOs: 969 or 970.

In some embodiments, the nucleic acid reduces expression of TGFBR2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the system comprises at least two nucleic acid sequences complementary to a nucleic acid encoding human TGFBR2 comprising the sequences as set forth in SEQ ID NOs: 969 and 970.

In some embodiments, the nucleic acid sequence complementary to human PTPN2 comprises a sequence set forth in SEQ ID NO: 968.

In some embodiments, the nucleic acid reduces expression of PTPN2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the at least one or more nucleic acids comprising a nucleic acid sequence at least 15 nucleotides in length complementary a portion thereof of human FAS, human TGFBR2, and human PTPN2 comprises a sequence as set forth in SEQ ID NO: 1252 or 972.

In some embodiments, the nucleic acid sequence complementary to a nucleic acid encoding human FAS reduces expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid, the nucleic acid sequence(s) complementary to a nucleic acid encoding human TGFBR2 reduces expression of TGFBR2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid, and the nucleic acid sequence complementary to a nucleic acid encoding human PTPN2 reduces expression of PTPN2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the system is encoded by: a nucleic acid sequence with at least 80%, 85%, 90%, 91%, 92%, 93%, 94%, 95%, 96%, 97%, 98%, 99%, or 100% identity to a sequence as set forth in SEQ ID NO: 1238; a nucleic acid sequence with at least 80%, 85%, 90%, 91%, 92%, 93%, 94%, 95%, 96%, 97%, 98%, 99%, or 100% identity to a sequence as set forth in SEQ ID NO: 1239; a nucleic acid sequence with at least 80%, 85%, 90%, 91%, 92%, 93%, 94%, 95%, 96%, 97%, 98%, 99%, or 100% identity to a sequence as set forth in SEQ ID NO: 1240; a nucleic acid sequence with at least 80%, 85%, 90%, 91%, 92%, 93%, 94%, 95%, 96%, 97%, 98%, 99%, or 100% identity to a sequence as set forth in SEQ ID NO: 1241; a nucleic acid sequence with at least 80%, 85%, 90%, 91%, 92%, 93%, 94%, 95%, 96%, 97%, 98%, 99%, or 100% identity to a sequence as set forth in SEQ ID NO: 1242; a nucleic acid sequence with at least 80%, 85%, 90%, 91%, 92%, 93%, 94%, 95%, 96%, 97%, 98%, 99%, or 100% identity to a sequence comprising nucleotides 481-7257 of SEQ ID NO: 1120; a nucleic acid sequence with at least 80%, 85%, 90%, 91%, 92%, 93%, 94%, 95%, 96%, 97%, 98%, 99%, or 100% identity to a sequence comprising nucleotides 481-7239 of SEQ ID NO: 1121; a nucleic acid sequence with at least 80%, 85%, 90%, 91%, 92%, 93%, 94%, 95%, 96%, 97%, 98%, 99%, or 100% identity to a sequence comprising nucleotides 481-7621 of SEQ ID NO: 1122; a nucleic acid sequence with at least 80%, 85%, 90%, 91%, 92%, 93%, 94%, 95%, 96%, 97%, 98%, 99%, or 100% identity to a sequence comprising nucleotides 481-7636 of SEQ ID NO: 1123; a nucleic acid sequence with at least 80%, 85%, 90%, 91%, 92%, 93%, 94%, 95%, 96%, 97%, 98%, 99%, or 100% identity to a sequence comprising nucleotides 481-7621 of SEQ ID NO: 1124; a nucleic acid sequence with at least 80%, 85%, 90%, 91%, 92%, 93%, 94%, 95%, 96%, 97%, 98%, 99%, or 100% identity to a sequence comprising nucleotides 24-7707 of SEQ ID NO: 1120; a nucleic acid sequence with at least 80%, 85%, 90%, 91%, 92%, 93%, 94%, 95%, 96%, 97%, 98%, 99%, or 100% identity to a sequence comprising nucleotides 24-7689 of SEQ ID NO: 1121; a nucleic acid sequence with at least 80%, 85%, 90%, 91%, 92%, 93%, 94%, 95%, 96%, 97%, 98%, 99%, or 100% identity to a sequence comprising nucleotides 24-8071 of SEQ ID NO: 1122; a nucleic acid sequence with at least 80%, 85%, 90%, 91%, 92%, 93%, 94%, 95%, 96%, 97%, 98%, 99%, or 100% identity to a sequence comprising nucleotides 24-8086 of SEQ ID NO: 1123; or a nucleic acid sequence with at least 80%, 85%, 90%, 91%, 92%, 93%, 94%, 95%, 96%, 97%, 98%, 99%, or 100% identity to a sequence comprising nucleotides 24-8071 of SEQ ID NO: 1124.

In some embodiments, the system is encoded by a nucleic acid comprising a sequence with at least 80%, 85%, 90%, 91%, 92%, 93%, 94%, 95%, 96%, 97%, 98%, 99%, or 100% identity to a sequence selected from the group consisting of the sequences set forth in SEQ ID NOs: 1120, 1121, 1122, 1123, 1124, 1238, 1239, 1240, 1241, or 1242.

In some embodiments, the target cell is a human cell.

In some embodiments, the target cell is a cancer cell expressing TMPRSS4 on its cell surface.

In some embodiments, the target cell is a cancer cell expressing SLC34A2 and TMPRSS4 on its cell surface.

In some embodiments, the cancer cell is a solid cancer cell or a liquid cancer cell.

In some embodiments, the cancer cell is a lung cell, optionally wherein the cancer cell is a non-small cell lung cancer (NSCLC) cell, ovarian cancer, cervical cancer, endometrial cancer, uterine cancer, pancreatic cancer, esophageal cancer, head and neck squamous cell cancer, thyroid cancer, bladder cancer, breast cancer, cholangiocarcinoma cancer, colon cancer, rectal cancer, kidney cancer, renal cell carcinoma, prostate cancer, stomach cancer, or gastric cancer.

In one aspect, provided herein are one or more nucleic acid(s) comprising at least one nucleic acid fragment comprising a nucleotide sequence encoding the system disclosed herein.

In some embodiments, the nucleic acid comprises a sequence with at least 80%, 85%, 90%, 91%, 92%, 93%, 94%, 95%, 96%, 97%, 98%, 99%, or 100% identity to a sequence as set forth in SEQ ID NO: 1238; a sequence with at least 80%, 85%, 90%, 91%, 92%, 93%, 94%, 95%, 96%, 97%, 98%, 99%, or 100% identity to a sequence as set forth in SEQ ID NO: 1239; a sequence with at least 80%, 85%, 90%, 91%, 92%, 93%, 94%, 95%, 96%, 97%, 98%, 99%, or 100% identity to a sequence as set forth in SEQ ID NO: 1240; a sequence with at least 80%, 85%, 90%, 91%, 92%, 93%, 94%, 95%, 96%, 97%, 98%, 99%, or 100% identity to a sequence as set forth in SEQ ID NO: 1241; a sequence with at least 80%, 85%, 90%, 91%, 92%, 93%, 94%, 95%, 96%, 97%, 98%, 99%, or 100% identity to a sequence as set forth in SEQ ID NO: 1242; at least 80%, 85%, 90%, 91%, 92%, 93%, 94%, 95%, 96%, 97%, 98%, 99%, or 100% identity to a sequence comprising nucleotides 481-7257 of SEQ ID NO: 1120; a sequence with at least 80%, 85%, 90%, 91%, 92%, 93%, 94%, 95%, 96%, 97%, 98%, 99%, or 100% identity to a sequence comprising nucleotides 481-7239 of SEQ ID NO: 1121; a sequence with at least 80%, 85%, 90%, 91%, 92%, 93%, 94%, 95%, 96%, 97%, 98%, 99%, or 100% identity to a sequence comprising nucleotides 481-7621 of SEQ ID NO: 1122; a sequence with at least 80%, 85%, 90%, 91%, 92%, 93%, 94%, 95%, 96%, 97%, 98%, 99%, or 100% identity to a sequence comprising nucleotides 481-7636 of SEQ ID NO: 1123; or a sequence with at least 80%, 85%, 90%, 91%, 92%, 93%, 94%, 95%, 96%, 97%, 98%, 99%, 100% identity to a sequence comprising nucleotides 481-7621 of SEQ ID NO: 1124; a sequence with at least 80%, 85%, 90%, 91%, 92%, 93%, 94%, 95%, 96%, 97%, 98%, 99%, or 100% identity to a sequence as set forth in SEQ ID NO: 1120; a sequence with at least 80%, 85%, 90%, 91%, 92%, 93%, 94%, 95%, 96%, 97%, 98%, 99%, or 100% identity to a sequence as set forth in SEQ ID NO: 1121; a sequence with at least 80%, 85%, 90%, 91%, 92%, 93%, 94%, 95%, 96%, 97%, 98%, 99%, or 100% identity to a sequence as set forth in SEQ ID NO: 1122, a sequence with at least 80%, 85%, 90%, 91%, 92%, 93%, 94%, 95%, 96%, 97%, 98%, 99%, or 100% identity to a sequence as set forth in SEQ ID NO: 1123, a sequence with at least 80%, 85%, 90%, 91%, 92%, 93%, 94%, 95%, 96%, 97%, 98%, 99%, or 100% identity to a sequence as set forth in SEQ ID NO: 1124; a sequence with at least 80%, 85%, 90%, 91%, 92%, 93%, 94%, 95%, 96%, 97%, 98%, 99%, or 100% identity to a sequence comprising nucleotides 24-7707 of SEQ ID NO: 1120; a sequence with at least 80%, 85%, 90%, 91%, 92%, 93%, 94%, 95%, 96%, 97%, 98%, 99%, or 100% identity to a sequence comprising nucleotides 24-7689 of SEQ ID NO: 1121; a sequence with at least 80%, 85%, 90%, 91%, 92%, 93%, 94%, 95%, 96%, 97%, 98%, 99%, or 100% identity to a sequence comprising nucleotides 24-8071 of SEQ ID NO: 1122; a sequence with at least 80%, 85%, 90%, 91%, 92%, 93%, 94%, 95%, 96%, 97%, 98%, 99%, or 100% identity to a sequence comprising nucleotides 24-8086 of SEQ ID NO: 1123; or a sequence with at least 80%, 85%, 90%, 91%, 92%, 93%, 94%, 95%, 96%, 97%, 98%, 99%, or 100% identity to a sequence comprising nucleotides 24-8071 of SEQ ID NO: 1124.

In some embodiments, the nucleic acid comprises a sequence selected from the group consisting of the sequences set forth in SEQ ID NOs: 1120, 1121, 1122, 1123, 1124, 1238, 1239, 1240, 1241 or 1242.

In one aspect, provided herein are one or more nucleic acid(s), wherein the one or more nucleic acid(s) encode:
a. a first chimeric polypeptide comprising a priming receptor comprising a first antigen-binding domain that specifically binds to human Solute Carrier Family 34 Member 2 (SLC34A2);
b. a second chimeric polypeptide comprising a chimeric antigen receptor (CAR) comprising a second antigen-binding domain that specifically binds to human Transmembrane protease, serine 4 (TMPRSS4).

In some embodiments, the nucleic acid(s) comprise comprising at least one nucleic acid sequence at least 15 nucleotides in length complementary to a portion thereof of:
a. a nucleic acid encoding human Fas Cell Surface Death Receptor (FAS) comprising the sequence set forth in SEQ ID NO: 964; and/or
b. a nucleic acid encoding human Transforming Growth factor (TGF)-β Receptor 2 (TGFBR2) comprising the sequence set forth in SEQ ID NO: 965; and/or
c. a nucleic acid encoding Phosphatase Non-Receptor Type 2 (PTPN2) comprising the sequence set forth in SEQ ID NO: 966.

In one aspect, provided herein are one or more nucleic acid(s), wherein the one or more nucleic acids encode:
a. a first chimeric polypeptide comprising a priming receptor comprising a first antigen-binding domain that specifically binds to human Solute Carrier Family 34 Member 2 (SLC34A2);
b. a second chimeric polypeptide comprising a chimeric antigen receptor (CAR) comprising a second antigen-binding domain that specifically binds to human Transmembrane protease, serine 4 (TMPRSS4); and
c. at least one nucleic acid sequence at least 15 nucleotides in length complementary to a portion thereof of:
   i. a nucleic acid encoding human Fas Cell Surface Death Receptor (FAS) comprising the sequence set forth in SEQ ID NO: 964; and/or
   ii. a nucleic acid encoding human Transforming Growth factor (TGF)-β Receptor 2 (TGFBR2) comprising the sequence set forth in SEQ ID NO: 965; and/or
   iii. a nucleic acid encoding human Protein Tyrosine Phosphatase Non-Receptor Type 2 (PTPN2) comprising the sequence set forth in SEQ ID NO: 966.

In some embodiments, the first antigen-binding domain comprises a heavy chain comprising a first variable heavy (VH) chain sequence comprising three heavy chain CDR sequences, CDR-H1, CDR-H2, and CDR-H3, of the VH sequences set forth in SEQ ID NOs: 1001, 1009, or 1015, and a first variable light (VL) chain sequence comprising three light chain CDR sequences, CDR-L1, CDR-L2, and CDR-L3, of the VL sequences set forth in SEQ ID NOs: 1005, 1013, 1125, or 1019, optionally wherein:
a. CDR-H1 comprises the sequence set forth in SEQ ID NO: 1002, CDR-H2 comprises the sequence set forth in SEQ ID NO: 1003, CDR-H3 comprises the sequence set forth in SEQ ID NO: 1004, CDR-L1 comprises the sequence set forth in SEQ ID NO: 1006, CDR-L2 comprises the sequence set forth in SEQ ID NO: 1007, and CDR-L3 comprises the sequence set forth in SEQ ID NO: 1008; or
b. CDR-H1 comprises the sequence set forth in SEQ ID NO: 1010, CDR-H2 comprises the sequence set forth in SEQ ID NO: 1011, CDR-H3 comprises the sequence set forth in SEQ ID NO: 1012, CDR-L1 comprises the sequence set forth in SEQ ID NO: 1006, CDR-L2 comprises the sequence set forth in SEQ ID NO: 1007, and CDR-L3 comprises the sequence set forth in SEQ ID NO: 1014; or
c. CDR-H1 comprises the sequence set forth in SEQ ID NO: 1016, CDR-H2 comprises the sequence set forth in SEQ ID NO: 1017, CDR-H3 comprises the sequence set forth in SEQ ID NO: 1018, CDR-L1 comprises the sequence set forth in SEQ ID NO: 1020, CDR-L2 comprises the sequence set forth in SEQ ID NO: 1021, and CDR-L3 comprises the sequence set forth in SEQ ID NO: 1022.

In some embodiments, the first VH chain sequence comprises the VH sequence set forth in SEQ ID NOs: 1001, 1009, or 1015.

In some embodiments, the second VL comprises the sequence set forth in SEQ ID NOs: 1005, 1013, 1125, or 1019.

In some embodiments, the first antigen-binding domain comprises the sequence set forth in SEQ ID NOs: 1107, 1108, or 1109.

In some embodiments, the second antigen-binding domain comprises a second variable heavy (VH) chain sequence comprising three heavy chain CDR sequences, CDR-H1, CDR-H2, and CDR-H3, of the VH sequences set forth in SEQ ID NOs: 319 or 326, and a second variable light (VL) chain sequence comprising three light chain CDR sequences, CDR-L1, CDR-L2, and CDR-L3, of the VL sequence set forth in SEQ ID NOs: 320 or 327, optionally wherein:
a. CDR-H1 comprises the sequence set forth in SEQ ID NO: 321, CDR-H2 comprises the sequence set forth in SEQ ID NO: 322, CDR-H3 comprises the sequence set forth in SEQ ID NO: 323, CDR-L1 comprises the sequence set forth in SEQ ID NO: 324, CDR-L2 comprises the sequence set forth in SEQ ID NO: 325, and CDR-L3 comprises the sequence set forth in SEQ ID NO: 16; and
b. CDR-H1 comprises the sequence set forth in SEQ ID NO: 193, CDR-H2 comprises the sequence set forth in SEQ ID NO: 80, CDR-H3 comprises the sequence set forth in SEQ ID NO: 328, CDR-L1 comprises the sequence set forth in SEQ ID NO: 329, CDR-L2 comprises the sequence set forth in SEQ ID NO: 330, and CDR-L3 comprises the sequence set forth in SEQ ID NO: 331.

In some embodiments, the second VH comprises the sequence as set forth in SEQ ID NO: 319 or 326.

In some embodiments, the second VL comprises the sequence set forth in SEQ ID NOS: 320 or 327.

In some embodiments, the second antigen binding domain comprises the sequence set forth in SEQ ID NO: 551 or 552.

In some embodiments, the at least one or more nucleic acids comprising a nucleic acid sequence at least 15 nucleotides in length complementary to a portion thereof of human FAS, human TGFBR2 and/or human PTPN2 are at least 16, 17, 18, 19, 20, 21, or 22 nucleotides in length.

In some embodiments, the at least one nucleic acid sequences are a short hairpin RNA (shRNA), a small interfering RNA (siRNA), a double stranded RNA (dsRNA), or an antisense oligonucleotide.

In some embodiments, the at least one nucleic acid sequences are shRNA.

In some embodiments, the at least one or more nucleic acids comprises a sequence with at least 80%, 85%, 90%, 91%, 92%, 93%, 94%, 95%, 96%, 97%, 98%, 99%, or 100% identity to a sequence as set forth in SEQ ID NO: 967.

In some embodiments, the at least one or more nucleic acid reduces expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the at least one or more nucleic acid comprises a sequence selected from the group consisting of the sequences set forth in SEQ ID NOs: 969 and/or 970.

In some embodiments, the at least one or more nucleic acid reduces expression of TGFBR2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the at least one or more nucleic acid comprise at least one nucleic acid sequence at least 15 nucleotides in length complementary a portion thereof of to a nucleic acid encoding human PTPN2 comprising the sequence set forth in SEQ ID NO: 966.

In some embodiments, the nucleic acid sequence complementary to human PTPN2 comprises a sequence as set forth in SEQ ID NO: 968.

In some embodiments, the nucleic acid reduces expression of PTPN2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the at least one or more nucleic acids comprising a nucleic acid sequence at least 15 nucleotides in length complementary a portion thereof of to human FAS, human TGFBR2, and human PTPN2 comprises a sequence as set forth in SEQ ID NO: 1252 or 972.

In some embodiments, the at least one or more nucleic acid sequence is encoded in at least one intron region of the nucleic acid.

In one aspect, provided herein are one or more nucleic acid(s) comprising the nucleic acid(s) disclosed herein.

In some embodiments, the nucleic acid comprises two or more nucleic acid fragments.

In some embodiments, the nucleic acid comprises an inducible promoter operably linked to the nucleotide sequence encoding the CAR, wherein the inducible promoter drives the inducible expression of the CAR.

In some embodiments, the nucleic acid comprises a constitutive promoter operably linked to the nucleotide sequence encoding the priming receptor, wherein the constitutive promoter drives constitutive expression of the priming receptor.

In some embodiments, the nucleic acid comprises an inducible promoter element operably linked to the nucleotide sequence encoding the chimeric antigen receptor and a constitutive promoter operably linked to the nucleotide sequence encoding the priming receptor.

In some embodiments, the constitutive promoter is an EF1α promoter.

In some embodiments, the constitutive promoter comprises a sequence as set forth in SEQ ID NO: 991.

In some embodiments, the inducible promoter comprises a YB-TATA promoter sequence and one or more Hepatocyte Nuclear Factor 1α (HNF1α) response element(s).

In some embodiments, the YB-TATA promoter sequence comprises a sequence as set forth in SEQ ID NO: 1246.

In some embodiments, the one or more Hepatocyte Nuclear Factor 1α (HNF1α) response element(s) comprises a sequence as set forth in SEQ ID NO: 1245.

In some embodiments, the inducible promoter comprises a sequence as set forth in SEQ ID NO: 992.

In some embodiments, the nucleic acid comprises, in a 5′ to 3′ direction,
a. the constitutive promoter;
b. the nucleotide sequence encoding priming receptor;
c. the inducible promoter element; and
d. the nucleotide sequence encoding chimeric antigen receptor.

In some embodiments, the nucleic acid comprises, in a 5′ to 3′ direction,
a. the inducible promoter element;
b. the nucleotide sequence encoding chimeric antigen receptor;
c. the constitutive promoter; and
d. the nucleotide sequence encoding priming receptor.

In some embodiments, the nucleic acid comprises, in a 5′ to 3′ direction,
a. a first constitutive promoter;
b. the nucleotide sequence encoding the priming receptor;
c. optionally, a second constitutive promoter;
d. the nucleotide sequence encoding the at least one nucleic acid complementary to human FAS, human TGFBR2, and/or human PTPN2;
e. the inducible promoter element; and
f. the optional nucleotide sequence encoding the chimeric antigen receptor.

In some embodiments, the nucleic acid comprises, in a 5′ to 3′ direction,
a. a first constitutive promoter;
b. the nucleotide sequence encoding the priming receptor;
c. optionally, a second constitutive promoter;
d. the nucleotide sequence encoding the first nucleic acid complementary to human FAS and/or the nucleotide sequence encoding the second nucleic acid complementary to human TGFBR2 and/or the nucleotide sequence encoding the third nucleic acid complementary to human PTPN2;
e. the nucleotide sequence encoding the first nucleic acid complementary to human FAS and/or the nucleotide sequence encoding the second nucleic acid complementary to human TGFBR2 and/or the nucleotide sequence encoding the third nucleic acid complementary to human PTPN2;
f. the nucleotide sequence encoding the first nucleic acid complementary to human FAS and/or the nucleotide sequence encoding the second nucleic acid complementary to human TGFBR2 and/or the nucleotide sequence encoding the third nucleic acid complementary to human PTPN2;
g. the inducible promoter element; and
h. the nucleotide sequence encoding the chimeric antigen receptor.

In some embodiments, the nucleic acid comprises, in a 5′ to 3′ direction,
a. the inducible promoter;
b. the nucleotide sequence encoding the chimeric antigen receptor;
c. a first constitutive promoter;
d. the nucleotide sequence encoding the first nucleic acid complementary to human FAS and/or the nucleotide sequence encoding the second nucleic acid complementary to human TGFBR2 and/or the nucleotide sequence encoding the third nucleic acid complementary to human PTPN2;
e. the nucleotide sequence encoding the first nucleic acid complementary to human FAS and/or the nucleotide sequence encoding the second nucleic acid complementary to human TGFBR2 and/or the nucleotide sequence encoding the third nucleic acid complementary to human PTPN2;
f. the nucleotide sequence encoding the first nucleic acid complementary to human FAS and/or the nucleotide sequence encoding the second nucleic acid complementary to human TGFBR2 and/or the nucleotide sequence encoding the third nucleic acid complementary to human PTPN2;
g. optionally, a second constitutive promoter; and
h. the nucleotide sequence encoding the priming receptor.

In some embodiments, the nucleic acid comprises a 5′ homology directed repair arm and a 3′ homology directed repair arm, both of which are complementary to an insertion site in a host cell chromosome.

In some embodiments, the nucleic acid comprises a woodchuck hepatitis virus post-translational regulatory element (WPRE).

In some embodiments, the WPRE is at the 3′ end of the nucleotide sequence encoding chimeric antigen receptor and at the 5′ end of the nucleotide sequence encoding priming receptor or wherein the WPRE is at the 3′ end of the nucleotide sequence encoding priming receptor and at the 5′ end of the nucleotide sequence encoding chimeric antigen receptor.

In some embodiments, the nucleic acid comprises synthetic polyA signal, an SV40 polyA signal, a human growth hormone (GH) polyA signal, or a bovine growth hormone (bGH) polyA signal.

In some embodiments, the nucleic acid is incorporated into an expression cassette or a vector for viral or non-viral delivery to a cell.

In some embodiments, the vector is for non-viral delivery and is, e.g., a non-viral vector.

In one aspect, provided herein are vectors comprising the nucleic acid disclosed herein.

In some embodiments, the 5′ and 3′ ends of the nucleic acid comprise nucleotide sequences that are homologous to genomic sequences flanking an insertion site in a genome of a primary cell.

In some embodiments, the insertion site is located at a genomic safe harbor (GSH) locus.

In some embodiments, the GSH locus is a GS94 locus (chr11: 128340000-128350000).

In some embodiments, the nucleotide sequences that are homologous to genomic sequences flanking the GS94 locus insertion site comprise nucleotides 24-473 and 7258-7707 of SEQ ID NO: 1120; nucleotides 24-473 and 7240-7689 of SEQ ID NO: 1121; nucleotides 24-473 and 7622-8071 of SEQ ID NO: 1122; nucleotides 24-473 and 7637-8086 of SEQ ID NO: 1123; or nucleotides 24-473 and 7622-8071 of SEQ ID NO: 1124.

In some embodiments, the nucleotide sequences that are homologous to genomic sequences flanking the GS94 locus insertion site comprise SEQ ID NOs: 1235 and 1236.

In some embodiments, nucleotide sequences comprise homology regions to the gRNA of the RNP complex used for inserting the nucleic acid into the genome of a cell.

In some embodiments, the sequences of the gRNA homology regions comprise SEQ ID NOs: 932 and 1237.

In one aspect, provided herein are isolated cells comprising:
a. the system disclosed herein;
b. at least one nucleic acid disclosed herein; and/or
c. the vector disclosed herein.

In some embodiments, the cell is an immune cell.

In one aspect, provided herein are isolated immune cells comprising:
a. the system disclosed herein;
b. at least one nucleic acid disclosed herein; and/or
c. the vector disclosed herein.

In some embodiments, the immune cell is a primary human immune cell.

In some embodiments, the primary immune cell is a natural killer (NK) cell, a T cell, a CD8+ T cell, a CD4+ T cell, a primary T cell, or a T cell progenitor.

In some embodiments, the primary immune cell is a primary T cell.

In some embodiments, the primary immune cell is a primary human T cell.

In one aspect, provided here are a population of isolated cells comprising a plurality of cells or immune cells disclosed herein.

In one aspect, provided here are pharmaceutical compositions comprising the isolated cells or immune cell disclosed herein or the population of isolated cells disclosed herein, and a pharmaceutically acceptable excipient.

In one aspect, provided here are pharmaceutical compositions comprising the nucleic acid disclosed herein or the vector disclosed herein, and a pharmaceutically acceptable excipient.

In one aspect, provided here are methods of editing a cell, comprising inserting the nucleic acid disclosed herein into a genome of the cell.

In some embodiments, the nucleic acid is inserted into a genomic safe harbor (GSH) locus.

In some embodiments, the GSH locus is a GS94 locus (chr11: 128340000-128350000).

In one aspect, provided here are methods of killing a target cell in a subject comprising administering the immune cell or population of immune cells disclosed herein to the subject, wherein the immune cell kills the target cell and/or triggers cytolysis of the target cell.

In one aspect, provided here are methods of inhibiting a target cell in a subject comprising administering the immune cell or population of immune cells disclosed herein to the subject, wherein the immune cell inhibits the target cell.

In some embodiments, the target cell expresses human TMRPSS4 or human TMPRSS4 and human SCL34A2.

In some embodiments, the target cell is a cancer cell.

In one aspect, provided here are methods of treating a disease in a human subject comprising administering the cells or immune cell or population of cells or immune cells disclosed herein or the pharmaceutical composition disclosed herein to the subject.

In some embodiments, the disease is cancer.

In one aspect, provided here are methods of treating cancer in a human subject, comprising administering the immune cell or population of immune cells disclosed herein or the pharmaceutical composition disclosed herein to the subject, wherein the immune cells are primary immune cells obtained from the subject.

In some embodiments, the cancer cells express human TMRPSS4 or human TMPRSS4 and human SCL34A2 on the cell membrane.

In one aspect, provided here are method of treating a disease in a subject comprising:
a. determining or having determined the presence of human SLC34A2-positive (SLC34A2+) cells in a cancer sample obtained from the subject; and/or
b. determining or having determined the presence of human TMPRSS4-positive (TMPRSS4+) cells in a cancer sample obtained from the subject; and
c. administering the cell or immune cell disclosed herein or the pharmaceutical composition disclosed herein to the subject.

In some embodiments, the cancer is a solid cancer or a liquid cancer.

In some embodiments, the cancer is non-small cell lung cancer (NSCLC), ovarian cancer, cervical cancer, endometrial cancer, uterine cancer, pancreatic cancer, esophageal cancer, head and neck squamous cell cancer, thyroid cancer, bladder cancer, breast cancer, cholangiocarcinoma cancer, colon cancer, rectal cancer, kidney cancer, renal cell carcinoma, prostate cancer, stomach cancer, or gastric cancer.

In some embodiments, the administration of the immune cell to the subject enhances an immune response in the subject or kills, or induces cytolysis, of the cancer cells.

In one aspect, provided here are methods of modulating the activity of a cell or immune cell comprising:
a. obtaining a cell or immune cell comprising
b. the system disclosed herein;
c. the nucleic acid disclosed herein; and/or
d. the vector disclosed herein; and
e. contacting the cell or immune cell with a target cell expressing SLC34A2 and TMPRSS4, wherein binding of the priming receptor to SLC34A2 on the target cell induces activation of the priming receptor and expression of the chimeric antigen receptor and wherein binding of the chimeric antigen receptor to TMPRSS4 on the target cell modulates the activity of the immune cell.

In some embodiments, the immune cell activity is cytolytic activity.

In some embodiments, the modulation of the immune cell activity comprises enhancing an immune response.

In some embodiments, the enhanced immune response is an adaptive immune response.

In some embodiments, the enhanced immune response is an innate immune response.

In some embodiments, the enhanced immune response is an increased expression of at least one cytokine or chemokine.

In some embodiments, the cytokine is interferon-gamma (IFNγ).

In some embodiments, the method comprises administering an immunotherapy to the subject concurrently with the cell or immune cell or subsequently to the cell or immune cell.

In one aspect, provided here are methods of inducing expression of a chimeric antigen receptor with a priming receptor in a cell comprising:
a. obtaining a cell or immune cell comprising
b. the system disclosed herein;
c. the nucleic acid disclosed herein; and/or
d. the vector disclosed herein; and
e. contacting the cell or immune cell with a cell expressing SLC34A2, wherein binding of the priming receptor to SLC34A2 on the cell induces activation of the priming receptor and expression of the chimeric antigen receptor.

In one aspect, provided here are nucleic acids comprising a nucleic acid sequence that has at least 80%, 85%, 90%, 91%, 92%, 93%, 94%, 95%, 96%, 97%, 98%, 99%, or 100% identity to SEQ ID NOs: 1120, 1121, 1122, 1123, 1124, 1238, 1239, 1240, 1241, or 1242, at least 80%, 85%, 90%, 91%, 92%, 93%, 94%, 95%, 96%, 97%, 98%, 99%, or 100% identity to a sequence comprising nucleotides 24-7707 of SEQ ID NO: 1120, at least 80%, 85%, 90%, 91%, 92%, 93%, 94%, 95%, 96%, 97%, 98%, 99%, or 100% identity to a sequence comprising nucleotides 24-7689 of SEQ ID NO: 1121, at least 80%, 85%, 90%, 91%, 92%, 93%, 94%, 95%, 96%, 97%, 98%, 99%, or 100% identity to a sequence comprising nucleotides 24-8071 of SEQ ID NO: 1122, at least 80%, 85%, 90%, 91%, 92%, 93%, 94%, 95%, 96%, 97%, 98%, 99%, or 100% identity to a sequence comprising nucleotides 24-8086 of SEQ ID NO: 1123, or at least 80%, 85%, 90%, 91%, 92%, 93%, 94%, 95%, 96%, 97%, 98%, 99%, or 100% identity to a sequence comprising nucleotides 24-8071 of SEQ ID NO: 1124; wherein the nucleic acid encodes a priming receptor comprising a first antigen binding domain that binds to SLC34A2 and a chimeric antigen receptor comprising a second antigen binding domain that binds to TMPRSS4.

In some embodiments, any difference in the nucleotide sequence as compared to SEQ ID NOs: 1120, 1121, 1122, 1123, 1124, 1238, 1239, 1240, 1241, 1242, or a sequence comprising nucleotides 24-7707 of SEQ ID NO: 1120, a sequence comprising nucleotides 24-7689 of SEQ ID NO: 1121, a sequence comprising nucleotides 24-8071 of SEQ ID NO: 1122, a sequence comprising nucleotides 24-8086 of SEQ ID NO: 1123, or a sequence comprising nucleotides 24-8071 of SEQ ID NO: 1124 does not affect the activity of any of the elements provided in Table 19.

In some embodiments, the nucleic acids comprise a sequence selected from the sequences as set forth in SEQ ID NOs: 1122, 1123, 1124, 1238, 1239, 1240, 1241, or 1242 or a sequence comprising nucleotides 24-7707 of SEQ ID NO: 1120, a sequence comprising nucleotides 24-7689 of SEQ ID NO: 1121, a sequence comprising nucleotides 24-8071 of SEQ ID NO: 1122, a sequence comprising nucleotides 24-8086 of SEQ ID NO: 1123, or a sequence comprising nucleotides 24-8071 of SEQ ID NO: 1124.

In one aspect, provided here are isolated human cells comprising the nucleic acid disclosed herein.

In some embodiments, the nucleic acid is inserted into a genomic safe harbor (GSH) locus of the cell.

In some embodiments, the GSH locus is a GS94 locus (chr11: 128340000-128350000).

In some embodiments, comprising a sequence selected from the sequences set forth in SEQ ID Nos: 1238, 1239, 1240, 1241 and 1242.

In one aspect, provided here are isolated human cells expressing a priming receptor comprising an antigen-binding domain that specifically binds to human SLC34A2 and a CAR comprising an antigen-binding domain that specifically binds to human TMPPRSS4, wherein binding of the priming receptor to SLC34A2 on the surface of a target cell and binding of the CAR to TMPRSS4 on the surface of a target cell induces lysis of the cell expressing TMPRSS4.

In some embodiments, where the cell expressing TMPRSS4 comprises SLC34A2 surface expression.

In one aspect, provided here are one or more nucleic acids comprising a nucleic acid sequence encoding a first cell surface receptor that specifically binds to human SLC34A2 and a nucleic acid sequence encoding a second cell surface receptor that specifically binds to human TMPRSS4, wherein binding of the first and second cell surface receptors to SLC34A2 and TMPRSS4 on the surface of a human cell, respectively, induces lysis of the human cell with TMPRSS4 on the surface.

In some embodiments, the first and the second cell surface receptor comprise the VH and VL of any of the SLC34A2 or TMPRSS4 antigen binding domains described herein, or their VH and VL CDRs.

### BRIEF DESCRIPTION OF THE DRAWINGS

These and other features, aspects, and advantages of the present invention will become better understood with regard to the following description, and accompanying drawings, where:

FIG. 1A shows binding of the indicated antibodies to TMPRSS4-expressing cells as determined by flow cytometry and measured by gMFI. FIG. 1B shows binding of TMPRSS4 antibodies to full-length catalytically inactive and truncated TMPRSS4 lacking the membrane-proximal domain as determined by flow cytometry and measured by gMFI. FIG. 1C shows flow cytometry histograms of binding of the TMPRSS4 antibodies to HEK293T cells engineered to express full length TMPRSS4 or parental HEK293T cells.

FIG. 2 is a schematic of an exemplary construct encoding a TMPRSS4 CAR.

FIG. 3A shows the percentages of T cells expressing CARs comprising the indicated TMPRSS4 antibodies as measured by flow cytometry (gMFI). FIG. 3B shows the overall expression of CARs comprising the indicated TMPRSS4 antibodies as measured by flow cytometry. FIG. 3C shows characterization of CD4+ cells into central memory T cells (T_{CM}), stem cell memory T cells (T_{SCM}), effector memory T cells (T_{EM}), or effector memory T cells re-expressing CD45RA (T_{EMRA}) based on flow cytometry detection of CCR7 and CD45RA. FIG. 3D shows characterization of CD8+ cells into T_{CM}, T_{SCM}, T_{EM}, or T_{EMRA} based on flow cytometry detection of CCR7 and CD45RA.

FIG. 4A shows surface expression of CD25 on T cells from donor CP5428 expressing CARs comprising the indicated TMPRSS4 antibodies. FIG. 4B shows surface expression of TIM3 on T cells from donor CP5428 expressing CARs comprising the indicated TMPRSS4 antibodies. FIG. 4C shows surface expression of CD25 on T cells from donor CP5917 expressing CARs comprising the indicated TMPRSS4 antibodies. FIG. 4D shows surface expression of TIM3 on T cells from donor CP5917 expressing CARs comprising the indicated TMPRSS4 antibodies.

FIG. 5A shows TMPRSS4 expression in LUDLU-1 and H1975 cell lines as determined by flow cytometry. FIG. 5B shows cytotoxicity (% killing) of LUDLU-1 and H1975 cell lines after incubation with TMPRSS4 CAR-expressing T cells at indicated effector: target (E:T) ratios.

FIG. 6A shows TMPRSS4 expression in H1975 cells with or without knockout of TMPRSS4 as determined by flow cytometry. FIG. 6B shows cytotoxicity (% killing) H1975 cells with or without knockout of TMPRSS4 after incubation with TMPRSS4 CAR-expressing T cells at indicated E:T ratios.

FIG. 7A shows secretion of IFNγ from TMPRSS4 CAR-expressing T cells following incubation with LUDLU-1 cells. FIG. 7B shows secretion of IFNγ from TMPRSS4 CAR-expressing T cells following incubation with parental H1975 cells. FIG. 7C shows secretion of IFNγ from TMPRSS4 CAR-expressing T cells following incubation with H1975 cells with TMPRSS4 knockout.

FIG. 8A shows secretion of TNFα from TMPRSS4 CAR-expressing T cells following incubation with LUDLU-1 cells. FIG. 8B shows secretion of TNFα from TMPRSS4 CAR-expressing T cells following incubation with parental H1975 cells. FIG. 8C shows secretion of TNFα from TMPRSS4 CAR-expressing T cells following incubation with H1975 cells with TMPRSS4 knockout.

FIG. 9 provides schematics of TMPRSS4 variants including the wild-type protein, a protein comprising a D290A mutation (indicated by X) that suppresses catalytic activity (“catalytic inactive”), and a truncated protein lacking the C-terminal serine protease domain (“cleaved/truncated”).

FIG. 10 shows CD69 surface expression in T cells expressing CARs comprising the indicated TMPRSS4 antibodies following co-culture with cells expressing the indicated TMPRSS4 protein.

FIG. 11 provides a schematic of the T cell engineering process and the first tier of the selection process.

FIG. 12 provides a schematic of functional sorting of T cells expressing a logic gate comprising an SLC34A2 primeR and a TMPRSS4 CAR following co-culture with indicated H1975 target cells.

FIG. 13A shows the percent knock-in (KI %) of genes in engineered T cells expressing a logic gate circuit in T cells from Donor A and Donor B. FIG. 13B shows the CAR expression/% KI (% conversion) of engineered T cells expressing a logic gate circuit in T cells from Donor A and Donor B. FIG. 13C shows the primeR expression in engineered T cells expressing a logic gate circuit in T cells from Donor A and Donor B.

FIG. 14 shows a schematic of the second tier of the selection process.

FIG. 15 shows SLC34A2 and TMPRSS4 expression in the indicated cell lines.

FIG. 16A shows the % killing (cytotoxicity) by engineered logic gate T cells from Donor B compared to the % killing (cytotoxicity) by engineered logic gate T cells from Donor A of TMPRSS4 knockout (KO) cells. FIG. 16B shows the % killing (cytotoxicity) by engineered logic gate T cells from Donor B compared to the % killing (cytotoxicity) by engineered logic gate T cells from Donor A of TMPRSS4^{hi} (high TMPRSS4 expression) cells. FIG. 16C shows the % killing (cytotoxicity) by engineered logic gate T cells from Donor B compared to the % killing (cytotoxicity) by engineered logic gate T cells from Donor A of SLC34A2^{hi} (high SLC34A2 expression)-TMPRSS4 knockout (KO) cells. FIG. 16D shows the % killing (cytotoxicity) by engineered logic gate T cells from Donor B compared to the % killing (cytotoxicity) by engineered logic gate T cells from Donor A of SLC34A2-TMPRSS4 expressing cells.

FIG. 17A shows IFNγ secretion by engineered T cells from donor A incubated with target cells expressing the TMPRSS4 protein. FIG. 17B shows IFNγ secretion by engineered T cells from donor B incubated with target cells expressing the TMPRSS4 protein.

FIG. 18A shows the result of the repetitive stimulation assay (RSA) of T cells from Donor A incubated with target cells expressing the TMPRSS4 protein. FIG. 18B shows the result of the RSA of T cells from Donor B incubated with target cells expressing the TMPRSS4 protein.

FIG. 19A shows a comparison of the % KI (cytotoxicity) by the engineered T cells from Donor A when incubated with cells expressing only TMPRSS4 (H1975-TMP^{hi}) as compared to the % KI (cytotoxicity) by the engineered T cells when incubated with cells expressing both SLC34A2 and TMPRSS4 (H975-dual). FIG. 19B shows a comparison of the % KI (cytotoxicity) by the engineered T cells from Donor B when incubated with cells expressing only TMPRSS4 (H1975-TMP^{hi}) as compared to the % KI (cytotoxicity) by the engineered T cells when incubated with cells expressing both SLC34A2 and TMPRSS4 (H1975-dual). The development candidates showed low % KI (cytotoxicity) when incubated with cells expressing only TMPRSS4 and high % KI (cytotoxicity) when incubated with cells expressing both SLC34A2 and TMPRSS4.

FIG. 20 shows the % cytotoxicity of T cells expressing the indicated logic gate when incubated with the indicated cell line.

FIG. 21 shows a schematic of the selection criteria for the lead logic gate candidates.

FIG. 22 shows additional characterization for leakiness and potency of the lead candidate logic gate T cells.

FIG. 23 shows the % cytotoxicity of T cells expressing the indicated logic gate when incubated with the indicated cell line.

FIG. 24 shows a diagram of exemplary logic gate circuits.

FIG. 25 shows binding of the indicated antibodies to cell expressing wild type and the D290A TMPRSS4 protein, as determined by flow cytometry and measured by gMFI.

FIG. 26A shows representative flow cytometry histograms of FAS (left panel) and TGFBR2 (right panel) expression in transgene-negative (PrimeR−) or transgene-positive (PrimeR+) T cells including the FAS/PTPN2/2xTFGBR2 shRNA module. PrimeR+ T cells showed reduced levels of FAS and TGFBR2 expression compared to PrimeR− T cells. FIG. 26B shows a Western blot image of PTPN2 expression in transgene-positive (PrimeR+) and transgene-negative (PrimeR−) T cells expressing the indicated logic gates including the FAS/PTPN2/2xTFGBR2 shRNA module. Transgene-positive T cells expressing the shRNA module had less PTPN2 expression as compared to transgene-negative T cells. FIG. 26C shows the quantified reduction of FAS (left), TGFBR2 (center), and PTPN2 expression (right) in engineered logic gate T cells expressing the indicated logic gate with the shRNA module. The data is presented as an average of 3 donors with standard deviation.

FIG. 27A shows cytotoxicity (% killing) of H2347 cells that endogenously express TMPRSS4 and SLC34A2 after incubation with the indicated engineered logic gate T cells at the indicated Effector: Target cell (E:T) ratios. FIG. 27B shows cytotoxicity (% killing) of H1648 cells that endogenously expresses TMPRSS4 and SLC34A2 after incubation with the indicated engineered logic gate T cells at the indicated E:T ratios. FIG. 27C shows the cytotoxicity (% killing) of H1975-SLC34A2/TMPRSS4 cells that expressed median levels of both target antigens after incubation with the indicated engineered logic gate T cells at the indicated E:T ratios

FIG. 28 shows quantification of the IFNγ production of the indicated logic gate T cells after incubation with a mixture of 786-O-SLC34A2 cells and aHEK293T-TMPRSS4 WT or HEK293T-TMPRSS4 D290A cells.

FIG. 29A shows a representative flow plot of priming receptor and CAR expression in the engineered logic gate T-cells after co-culture with a 786-O parental cell line that does not express SLC34A2 (left) and a SLC34A2 positive cell line (right). FIG. 29B shows prime antigen dependent % CAR expression induction (left) and CAR expression (mean fluorescent intensity, MFI) (right) for the indicated engineered logic gate T cells over time when co-cultured with a cell line expressing the SLC34A2 priming antigen. FIG. 29C shows representative flow plots of CAR and priming receptor expression in engineered logic gate T cells in an “ON” state (left) after co-culture with cells expressing the priming antigen (left, “ON” state) and after co-culture with cells lacking expression of the priming antigen (right, “OFF” state). FIG. 29D shows prime antigen dependent % CAR expression induction (left) and CAR expression (mean fluorescent intensity, MFI) (right) over time after removal of the priming antigen.

FIG. 30 shows long term cytotoxicity of T cells in a repetitive stimulation assay (RSA). The indicated logic gate T cells were incubated with a H1975-SLC34A2/TMPRSS4 overexpressing target cell line and rechallenged with new target cells over time. Lines indicate target cell restimulation times. Target cell killing was determined by measuring the fluorescence intensity of GFP expressed by the target cells.

FIG. 31A shows a priming antigen heterogeneity cytotoxicity assay, where the indicated logic gate T cells were incubated at a 1:1 E:T ratio with a mixture of H1975-EFG-SLC34A2/TMPRSS4 and H1975-EFG-TMPRSS4 cells at the indicated target cell heterogeneity ratios. FIG. 31B shows a priming antigen heterogeneity cytotoxicity assay, where the indicated logic gate T cells were incubated at a 1:3 E:T ratio with a mixture of H1975-EFG-SLC34A2/TMPRSS4 and H1975-EFG-TMPRSS4 cells at the indicated target cell heterogeneity ratios. FIG. 31C shows a priming antigen heterogeneity cytotoxicity assay, where the indicated logic gate T cells were incubated at a 1:9 E:T ratio with a mixture of H1975-EFG-SLC34A2/TMPRSS4 D20A and H1975-EFG-TMPRSS4 cells at the indicated target cell heterogeneity ratios.

FIG. 32 shows in vivo tumor-growth inhibition over time of H1975-nEFG-SLC43A2-TMPRSS4 lung adenocarcinoma tumors after treatment with the indicated logic gate T cells.

FIG. 33A shows in vivo tumor-growth inhibition over time of H1975 tumors expressing SLC34A2-TMPRSS4 in a dual flank lung adenocarcinoma xenograft model after treatment with the indicated logic gate T cells. FIG. 33B shows in vivo tumor-growth inhibition over time of tumors expressing only TMPRSS4 in a dual flank lung adenocarcinoma xenograft model after treatment with the indicated logic gate T cells.

FIG. 34A shows the % reduction of FAS expression by flow cytometry in LG 47 T cells expressing the indicated shRNA. FIG. 34B shows long term cytotoxicity of the indicated T cells in a repetitive stimulation assay (RSA), after incubation of the indicated logic gate T cells with a H1975-SLC34A2/TMPRSS4+FASL overexpressing target cell line and rechallenged with new target cells over time. Arrows indicate target cell restimulation times. FIG. 34C shows a representative histogram of Fas (left panel) and FasL (right panel) expression in the indicated cell lines. Batimastat treatment was used to inhibit cleavage of cell surface expressed FASL.

FIG. 35A shows long term cytotoxicity and T cell proliferation of the indicated T cells in a repetitive stimulation assay (RSA) after incubation of the indicated LG T cells with a K562-SLC34A2/TMPRSS4 target cell line and restimulated over time. Arrows indicate target cell restimulation times. T cell counts are shown in white shading with a solid line, target cell counts are shown in gray shading with a dotted line. FIG. 35B shows T cell proliferation across RSA rounds in the indicated logic gate T cells from 3 donors. Data for both is the mean with standard deviation across 3 donors.

FIG. 36A shows the % reduction of TGFBR2 expression by flow cytometry in T cells expressing the Logic Gate 47 priming receptor and CAR and the indicated shRNA. FIG. 36B shows quantification of phosphorylated SMAD (gMFI) in in transgene-positive (PrimeR+) and transgene-negative (PrimeR−) T cells expressing the Logic Gate 47 priming receptor and CAR and the indicated shRNA, with the addition of TGFβ (left) or without TGFβ (right). FIG. 36C shows long term cytotoxicity of the indicated T cells in a repetitive stimulation assay (RSA). The indicated logic gate T cells were incubated with a H1975-SLC34A2/TMPRSS4 overexpressing target cell line and rechallenged with new target cells over time. The T cells were restimulated with the target cells at times indicated by gray arrows.

FIG. 37 shows in vivo tumor-growth inhibition over time of H1975-nEFG-SLC43A2-TMPRSS4 lung adenocarcinoma tumors after treatment with the indicated logic gate T cells derived from two donors. The logic gate T cells expressed the full FAS/PTPN2/2xTGFBR2 shRNA, or a quad luciferase (quad luc) control shRNA module.

### DETAILED DESCRIPTION OF THE INVENTION

The present disclosure provides polypeptide systems of synthetic transcriptional modulators that comprise antigen binding domains that bind to SLC34A2 and synthetic immune receptors (e.g., CARs) that comprise antigen binding domains that bind to TMPRSS4. The present disclosure also provides nucleic acids encoding the systems, cells comprising the polypeptide systems, and methods of making and/or using the cells.

### Definitions

Terms used in the claims and specification are defined as set forth below unless otherwise specified.

It must be noted that, as used in the specification and the appended claims, the singular forms “a,” “an” and “the” include plural referents unless the context clearly dictates otherwise.

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). For example, an antibody that “selectively binds” or “specifically binds” an antigen is an antigen-binding moiety that binds the antigen with high affinity and does not significantly bind other unrelated antigens. Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule.

“Affinity” refers to the strength of the sum total of non-covalent interactions between a single binding site of a molecule (e.g., an antibody or antigen binding protein) and its binding partner (e.g., an antigen or epitope). Unless indicated otherwise, as used herein, “affinity” refers to intrinsic binding affinity, which reflects a 1:1 interaction between members of a binding pair (e.g., antibody and antigen or epitope). The affinity of a molecule X for its partner Y can be represented by the dissociation equilibrium constant (KD). The kinetic components that contribute to the dissociation equilibrium constant are described in more detail below. Affinity can be measured by common methods known in the art, including, but not limited to, surface plasmon resonance (SPR) technology (e.g., BIACORE®) or biolayer interferometry (e.g., FORTEBIO®).

The term “complementarity determining region” “CDR,” as used herein, refers to each of the regions of an antibody variable domain which are hypervariable in sequence and/or form structurally defined loops (“hypervariable loops,” “hypervariable region,” or “HVR”). Generally, native four-chain antibodies comprise six CDRs; three in the VH (HCDR1/CDR-H1, HCDR2/CDR-H2, and HCDR3/CDR-H3), and three in the VL (LCDR1/CDR-L1, LCDR2/CDR-L2, and LCDR3/CDR-L3). With the exception of CDR1 in VH, CDRs generally comprise the amino acid residues that form the hypervariable loops. Complementarity determining regions (CDRs) are also referred to as “hypervariable regions” or “HVRs”, and these terms are used herein interchangeably in reference to portions of the variable region that form the antigen-binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); Martin (Enhanced Chothia) Abhinandan and Martin, Mol Immunol. 2008 August; 45(14):3832-9; MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegger and Plückthun, J. Mol. Biol., 2001, 309:657-70 (“AHo” numbering scheme); each of which is incorporated by reference in its entirety.

Table 1 provides the positions of LCDR1/CDR-L1, LCDR2/CDR-L2, LCDR3/CDR-L3, HCDR1/CDR-H1, HCDR2/CDR-H2, and HCDR3/CDR-H3 as identified by the Kabat and Chothia schemes. For HCDR1/CDR-H1, residue numbering is provided using both the Kabat and Chothia numbering schemes.

CDRs may be assigned, for example, using antibody numbering software, such as Abnum, available at bioinf.org.uk/abs/abnum/, and described in Abhinandan and Martin, Immunology, 2008, 45:3832-3839, incorporated by reference in its entirety. Descriptions of the various antibody numbering schemes are available at bioinf.org.uk/abs/info.html and the AbYsis program.

TABLE 1
Residues in CDRs according to Kabat and Chothia numbering schemes.

| CDR | Kabat | Chothia | AbM | Contact | IMGT |
|---|---|---|---|---|---|
| L1 | L24-L34 | L24-L34 | L24-L34 | L30-L36 | L27-L32 |
| L2 | L50-L56 | L50-L56 | L50-L56 | L46-L55 | L50-L51 |
| L3 | L89-L97 | L89-L97 | L89-L97 | L89-L96 | L89-L97 |
| H1 (Kabat Numbering) | H31-H35B | H26-H32 or H34* | H26-H35B | H30-H35B | H26-H35B |
| H1 (Chothia/Martin | H31-H35 | H26-H32 | H26-H35 | H30-H35 | H26-H33 |
| Numbering) | | | | | |
| H2 | H50-H65 | H52-H56 | H50-H58 | H47-H58 | H51-H56 |
| H3 | H95-H102 | H95-H102 | H95-H102 | H93-H101 | H93-H102 |

*The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

TABLE 2
Example Conservative Substitutions

| | | Specific Example |
|---|---|---|
| Original | Example Substitutions | Substitutions |
| Ala (A) | Val, Leu, Ile | Val |
| Arg (R) | Lys, Gln, Asn | Lys |
| Asn (N) | Gln, His, Asp, Lys, Arg | Gln |
| Asp (D) | Glu, Asn | Glu |
| Cys (C) | Ser, Ala | Ser |
| Gln (Q) | Asn, Glu | Asn |
| Glu (E) | Asp, Gln | Asp |
| Gly (G) | Ala | Ala |
| His (H) | Asn, Gln, Lys, Arg | Arg |
| Ile (I) | Leu, Val, Met, Ala, Phe | Leu |
| Leu (L) | Norleucine, Ile, Val, Met, Ala | Ile |
| Lys (K) | Arg, Gln, Asn | Arg |
| Met (M) | Leu, Phe, Ile | Leu |
| Phe (F) | Leu, Val, Ile, Ala, Tyr | Tyr |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala | Leu |

As used herein, the term “single-chain” refers to a molecule comprising amino acid monomers linearly linked by peptide bonds. In a particular such embodiment, the C-terminus of a Fab light chain is connected to the N-terminus of a Fab heavy chain in a single-chain Fab molecule. As described in more detail herein, an scFv has a variable domain of light chain (VL) connected from its C-terminus to the N-terminal end of a variable domain of heavy chain (VH) by a polypeptide chain or linker. Alternately an scFv comprises a polypeptide chain wherein the C-terminal end of a VH is connected to the N-terminal end of a VL by a polypeptide chain or linker.

The “Fab fragment” (also referred to as fragment antigen-binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen-binding. Fab′ fragments differ from Fab fragments by the addition of a few residues at the carboxy terminus of the heavy chain CH1 domain including one or more cysteines from the antibody hinge region.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with β-mercaptoethanol.

“Fv” fragments comprise a non-covalently-linked dimer of one heavy chain variable domain and one light chain variable domain.

The “Single-chain Fv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds., Springer-Verlag, New York, pp. 269-315 (1994). HER2 antibody scFv fragments are described in WO93/16185; U.S. Pat. Nos. 5,571,894; and 5,587,458.

The term “single domain antibody” or “sdAb” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain antibodies are also known as sdAbs or nanobodies. Sdabs are fairly stable and easy to express as fusion partner with the Fc chain of an antibody (Harmsen M M, De Haard H J (2007). “Properties, production, and applications of camelid single-domain antibody fragments”. Appl. Microbiol Biotechnol. 77(1): 13-22). As used herein, the term “single-chain” refers to a molecule comprising amino acid monomers linearly linked by peptide bonds. In a particular such embodiment, the C-terminus of the Fab light chain is connected to the N-terminus of the Fab heavy chain in the single-chain Fab molecule. As described in more detail herein, an scFv has a variable domain of light chain (VL) connected from its C-terminus to the N-terminal end of a variable domain of heavy chain (VH) by a polypeptide chain. Alternately the scFv comprises of polypeptide chain where in the C-terminal end of the VH is connected to the N-terminal end of VL by a polypeptide chain.

As used herein, the term “gene” refers to the basic unit of heredity, consisting of a segment of DNA arranged along a chromosome, which codes for a specific protein or segment of protein. A gene typically includes a promoter, a 5′ untranslated region, one or more coding sequences (exons), optionally introns, and a 3′ untranslated region. The gene may further comprise a terminator, enhancers and/or silencers.

The terms “genetic engineering,” “gene editing,” or “genome editing”, as used herein, refer to a type of genetic manipulation in which DNA is inserted, replaced, or removed from the genome using artificially manipulated nucleases or “molecular scissors”. It is a useful tool for elucidating the function and effect of sequence-specific genes or proteins or altering cell behavior (e.g., for therapeutic purposes).

Gene editing, as contemplated herein, may involve a gene (or nucleotide sequence) knock-in or knock-out. As used herein, the term “knock-in” refers to an addition of a DNA sequence, or fragment thereof into a genome. Such DNA sequences to be knocked-in may include an entire gene or genes, may include regulatory sequences associated with a gene or any portion or fragment of the foregoing. For example, a polynucleotide donor construct encoding a recombinant protein may be inserted into the genome of a cell carrying a mutant gene. In some embodiments, a knock-in strategy involves substitution of an existing sequence with the provided sequence, e.g., substitution of a mutant allele with a wild-type copy. On the other hand, the term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant (e.g., non-coding) sequence.

Currently available genome editing tools include zinc finger nucleases (ZFN) and transcription activator-like effector nucleases (TALENs) to incorporate genes at safe harbor loci (e.g., the adeno-associated virus integration site 1 (AAVS1) safe harbor locus or any other safe harbor loci disclosed herein). The DICE (dual integrase cassette exchange) system utilizing phiC31 integrase and Bxb1 integrase is a tool for target integration. Additionally, clustered regularly interspaced short palindromic repeat/Cas (CRISPR/Cas) techniques can be used for targeted gene insertion. Site specific gene editing approaches can include homology dependent mechanisms or homology independent mechanisms. All methods known in the art for targeted insertion of gene sequences are contemplated in the methods described herein to insert constructs at gene targets or safe harbor loci.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, Cas9 in complex with guide and activating RNA to recognize and cleave foreign nucleic acid. Guide RNAs having the activity of both a guide RNA and an activating RNA are also known in the art. In some cases, such dual activity guide RNAs are referred to as a small guide RNA (sgRNA).

As used herein, a polypeptide referred to as a “Cas endonuclease” or having “Cas endonuclease activity” refers to a CRISPR-related (Cas) polypeptide encoded by a Cas gene, wherein a Cas polypeptide is a target DNA sequence that can be cleaved when operably linked to one or more guide polynucleotides (see, e.g., U.S. Pat. No. 8,697,359). Also included in this definition are variants of Cas endonuclease that retain guide polynucleotide-dependent endonuclease activity. The Cas endonuclease used in the donor DNA insertion method detailed herein is an endonuclease that introduces double-strand breaks into DNA at the target site (e.g., within the target locus or at the safe harbor site).

As used herein, the term “Cas9” refers to an RNA-mediated nuclease (e.g., of bacterial or archeal origin, or derived therefrom). Exemplary RNA-mediated nucleases include the foregoing Cas9 proteins and homologs thereof, and include but are not limited to, CPF1 (See, e.g., Zetsche et al., Cell, Volume 163, Issue 3, p759-771, 22 Oct. 2015). Similarly, as used herein, the term “Cas9 ribonucleoprotein” complex and the like refers to a complex between the Cas9 protein, and a crRNA (e.g., guide RNA or small guide RNA), the Cas9 protein and a trans-activating crRNA (tracrRNA), the Cas9 protein and a small guide RNA, or a combination thereof (e.g., a complex containing the Cas9 protein, a tracrRNA, and a crRNA guide RNA). Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5):726-737; Nat. Rev. Microbiol. 2011 June; 9 (6): 467-477; Hou, et al., Proc Natl Acad Sci USA. 2013 Sep. 24; 110(39):15644-9; Sampson et al., Nature. 2013 May 9; 497(7448):254-7; and Jinek, et al., Science. 2012 Aug. 17; 337(6096):816-21. The Cas9 nuclease domain can be optimized for efficient activity or enhanced stability in the host cell.

As used herein, the term “guide polynucleotide” relates to a polynucleotide sequence capable of complexing with a Cas endonuclease and allowing the Cas endonuclease to recognize and cleave a DNA target site. The guide polynucleotide can be a single molecule or a double molecule. The guide polynucleotide sequence can be an RNA sequence, a DNA sequence, or a combination thereof (RNA-DNA combination sequence). A guide polynucleotide comprising only ribonucleic acid is also referred to as “guide RNA”. In some embodiments, a polynucleotide donor construct is inserted at a safe harbor locus using a guide RNA (gRNA) in combination with a Cas endonuclease (e.g., Cas9 endonuclease).

As used herein, the term “homology directed repair” or HDR refers to a cellular process in which cut or nicked ends of a DNA strand are repaired by polymerization from a homologous template nucleic acid. Thus, the original sequence is replaced with the sequence of the template. The homologous template nucleic acid can be provided by homologous sequences elsewhere in the genome (sister chromatids, homologous chromosomes, or repeated regions on the same or different chromosomes). Alternatively, an exogenous template nucleic acid can be introduced to obtain a specific HDR-induced change of the sequence at the target site. In this way, specific mutations can be introduced at the cut site.

As used herein, the term “non-homologous end joining” or NHEJ refers to a cellular process in which cut or nicked ends of a DNA strand are directly ligated without the need for a homologous template nucleic acid. NHEJ can lead to the addition, the deletion, substitution, or a combination thereof, of one or more nucleotides at the repair site.

As used herein, the term “integration” refers to the process of stably inserting one or more nucleotides of a construct into the cell genome, i.e., covalently linking to a nucleic acid sequence in the chromosomal DNA of the cell. It may also refer to nucleotide deletions at a site of integration. Where there is a deletion at the insertion site, “integration” may further include substitution of the endogenous sequence or nucleotide deleted with one or more inserted nucleotides.

As used herein, the term “locus” refers to a specific, fixed physical location on a chromosome where a gene or genetic marker is located.

The term “safe harbor locus” refers to a locus at which genes or genetic elements can be incorporated without disruption to expression or regulation of adjacent genes. These safe harbor loci are also referred to as safe harbor sites (SHS) or genomic safe harbor (GSH) sites. As used herein, a safe harbor locus refers to an “integration site” or “knock-in site” at which a sequence encoding a transgene, as defined herein, can be inserted. In some embodiments the insertion occurs with replacement of a sequence that is located at the integration site. In some embodiments, the insertion occurs without replacement of a sequence at the integration site. Examples of integration sites contemplated are provided in Table 9.

A “chemotherapeutic agent” refers to a chemical compound useful in the treatment of cancer. Chemotherapeutic agents include “anti-hormonal agents” or “endocrine therapeutics” which act to regulate, reduce, block, or inhibit the effects of hormones that can promote the growth of cancer.

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease.

As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “complementary” or “complementarity” refers to specific base pairing between nucleotides or nucleic acids. Complementary nucleotides are, generally, A and T (or A and U), and G and C. The guide RNAs described herein can comprise sequences, for example, DNA targeting sequence that are perfectly complementary or substantially complementary (e.g., having 1-4 mismatches) to a genomic sequence in a cell.

The term “composition” refers to a mixture that contains, e.g., an engineered cell or protein contemplated herein. In some embodiments, the composition may contain additional components, such as adjuvants, stabilizers, excipients, and the like. The term “composition” or “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

As used herein, the term “developmental cell states” refers to, for example, states when the cell is inactive, actively expressing, differentiating, senescent, etc. developmental cell state may also refer to a cell in a precursor state (e.g., a T cell precursor).

The term “ameliorating” refers to any therapeutically beneficial result in the treatment of a disease state, e.g., a cancer disease state, lessening in the severity or progression, remission, or cure thereof.

As used herein, the term “effective amount” refers to the amount of a compound (e.g., a compositions described herein, cells described herein) sufficient to effect beneficial or desired results. An effective amount can be administered in one or more administrations, applications or dosages and is not intended to be limited to a particular formulation or administration route.

As used herein the term “expression cassette” is a polynucleotide construct, generated recombinantly or synthetically, comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in an expression vector.

As used, the term “encoding” refers to a sequence of nucleotides which codes for a protein or polypeptide of interest or non-protein coding sequences. The nucleic acid sequence may be either a molecule of DNA or RNA. In preferred embodiments, the molecule is a DNA molecule. In other preferred embodiments, the molecule is a RNA molecule. When present as a RNA molecule, it will comprise sequences which direct the ribosomes of the host cell to start translation (e.g., a start codon, ATG) and direct the ribosomes to end translation (e.g., a stop codon). Between the start codon and stop codon is an open reading frame (ORF). Such terms are known to one of ordinary skill in the art. Non-protein coding sequences include, but are not limited to, short hairpin RNA (shRNA), small interfering RNA (siRNA), double stranded RNA (dsRNA), or antisense oligonucleotides.

As used herein, a single-stranded DNA template or a double-stranded DNA template refers to a DNA oligonucleotide that can be used by a cell as a template for HDR. Generally, the single-stranded DNA template or a double-stranded DNA template has at least one region of homology to a target site. In some cases, the single-stranded DNA template or double-stranded DNA template has two homologous regions flanking a region that contains a heterologous sequence to be inserted at a target cut site.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

As used herein, the term “to insert” or “inserting” refers to process of integrating a nucleotide sequence into the genome of a cell, such as at a target locus or safe harbor site. The term “insert” also can be used to refer to the genes or genetic elements that are incorporated at the target locus or safe harbor site using, for example, homology-directed repair (HDR) CRISPR/Cas (e.g., CRISPR/Cas9) genome-editing or other methods for inserting nucleotide sequences into a genomic region known to those of ordinary skill in the art.

As used herein, the phrase “introducing” in the context of introducing into a cell a nucleic acid or a complex comprising a nucleic acid, for example, an RNP-DNA template complex, refers to the translocation of the nucleic acid sequence or the RNP-DNA template complex from outside a cell to inside the cell. In some cases, introducing refers to translocation of the nucleic acid or the complex from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

As used herein, the term “operably linked” or “operatively linked” refers to the binding of a nucleic acid sequence to a single nucleic acid fragment such that one function is affected by the other. For example, if a promoter is capable of affecting the expression of a coding sequence or functional RNA (i.e., the coding sequence or functional RNA is under transcriptional control by the promoter), the promoter is operably linked thereto. Coding sequences can be operably linked to control sequences in both sense and antisense orientation.

As used herein, a “polynucleotide donor construct” refers to a nucleotide sequence (e.g., DNA sequence) that is genetically inserted into a polynucleotide and is exogenous to that polynucleotide. The polynucleotide donor construct is transcribed into RNA and optionally translated into a polypeptide. The polynucleotide donor construct can include prokaryotic sequences, cDNA from eukaryotic mRNA, genomic DNA sequences from eukaryotic (e.g., mammalian) DNA, and synthetic DNA sequences. For example, the polynucleotide donor construct can be a miRNA, shRNA, natural polypeptide (i.e., a naturally occurring polypeptide) or fragment thereof or a variant polypeptide (e.g., a natural polypeptide having less than 100% sequence identity with the natural polypeptide) or fragments thereof.

As used herein, the term “promoter” refers to a nucleotide sequence (e.g., DNA sequence) capable of controlling the expression of a coding sequence or functional RNA. The promoter sequence consists of proximal and more distal upstream elements, the latter elements often referred to as enhancers. A promoter can be derived from natural genes in its entirety, can be composed of different elements from different promoters found in nature, and/or may comprise synthetic DNA segments. A promoter, as contemplated herein, can be endogenous to the cell of interest or exogenous to the cell of interest. It is appreciated by those skilled in the art that different promoters can induce gene expression in different tissue or cell types, or at different developmental stages, or in response to different environmental conditions. As is known in the art, a promoter can be selected according to the strength of the promoter and/or the conditions under which the promoter is active, e.g., constitutive promoter, strong promoter, weak promoter, inducible/repressible promoter, tissue specific or developmentally regulated promoters, cell cycle-dependent promoters, and the like.

A promoter can be an inducible promoter (e.g., a heat shock promoter, tetracycline-regulated promoter, steroid-regulated promoter, metal-regulated promoter, estrogen receptor-regulated promoter, etc.). In some embodiments, an inducible promoter comprises one or more inducible response elements (e.g., Hepatocyte Nuclear Factor 1α (HNF1α) response elements) operably linked to a basal promoter element (e.g., a YB-TATA promoter). The promoter can be a constitutive promoter (e.g., CMV promoter, UBC promoter). In some embodiments, the promoter can be a spatially restricted and/or temporally restricted promoter (e.g., a tissue specific promoter, a cell type specific promoter, etc.). See for example US Publication 2018/0127786, the disclosure of which is herein incorporated by reference in its entirety.

As used herein, the term “transgene” refers to a polynucleotide that has been transferred naturally, or by any of a number of genetic engineering techniques from one organism to another. It is optionally translated into a polypeptide. It is optionally translated into a recombinant protein. A “recombinant protein” is a protein encoded by a gene-recombinant DNA—that has been cloned in a system that supports expression of the gene and translation of messenger RNA (see expression system). The recombinant protein can be a therapeutic agent, e.g., a protein that treats a disease or disorder disclosed herein. As used, transgene can refer to a polynucleotide that encodes a polypeptide.

The terms “vector” and “plasmid” are used interchangeably and as used herein refer to polynucleotide vehicles useful to introduce genetic material into a cell. Vectors can be linear or circular. Vectors can integrate into a target genome of a host cell or replicate independently in a host cell. Vectors can comprise, for example, an origin of replication, a multicloning site, and/or a selectable marker. An expression vector typically comprises an expression cassette. Vectors and plasmids include, but are not limited to, integrating vectors, prokaryotic plasmids, eukaryotic plasmids, plant synthetic chromosomes, episomes, cosmids, and artificial chromosomes.

The term “in vivo” refers to processes that occur in a living organism.

The term “in situ” refers to processes that occur in a living cell growing separate from a living organism, e.g., growing in tissue culture.

As used herein, the term “ex vivo” generally includes experiments or measurements made in or on living tissue, preferably in an artificial environment outside the organism, preferably with minimal differences from natural conditions.

As used herein, the phrase “hematopoietic stem cell” refers to a type of stem cell that can give rise to a blood cell. Hematopoietic stem cells can give rise to cells of the myeloid or lymphoid lineages, or a combination thereof. Hematopoietic stem cells are predominantly found in the bone marrow, although they can be isolated from peripheral blood, or a fraction thereof. Various cell surface markers can be used to identify, sort, or purify hematopoietic stem cells. In some cases, hematopoietic stem cells are identified as c-Kit⁺ and lin. In some cases, human hematopoietic stem cells are identified as CD34⁺, CD59⁺, Thy1/CD90⁺, CD38^{lo/-}, C-kit/CD117⁺, lin. In some cases, human hematopoietic stem cells are identified as CD34⁻, CD59⁺, Thy1/CD90⁺, CD38^{lo/-}, C-kit/CD117⁺, lin⁻. In some cases, human hematopoietic stem cells are identified as CD133⁺, CD59⁺, Thy1/CD90⁺, CD38^{lo/-}, C-kit/CD117⁺, lin⁻. In some cases, mouse hematopoietic stem cells are identified as CD34^{lo/-}, SCA-1⁺, Thy1^{+/lo}, CD38⁺, C-kit⁺, lin⁻. In some cases, the hematopoietic stem cells are CD150⁺CD48⁻CD244⁻.

As used herein, the phrase “hematopoietic cell” refers to a cell derived from a hematopoietic stem cell. The hematopoietic cell may be obtained or provided by isolation from an organism, system, organ, or tissue (e.g., blood, or a fraction thereof). Alternatively, an hematopoietic stem cell can be isolated and the hematopoietic cell obtained or provided by differentiating the stem cell. Hematopoietic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes.

As used herein, the phrase “immune cell” is inclusive of all cell types that can give rise to immune cells, including hematopoietic cells such hematopoietic stem cells, pluripotent stem cells, and induced pluripotent stem cells (iPSCs). In some embodiments, the immune cell is a B cell, macrophage, a natural killer (NK) cell, an induced pluripotent stem cell (iPSC), a human pluripotent stem cell (HSPC), a T cell or a T cell progenitor or dendritic cell. In some embodiments, the cell is an innate immune cell.

As used herein, the terms “T lymphocyte” and “T cell” are used interchangeably and refer to cells that have completed maturation in the thymus, and identify certain foreign antigens in the body. The terms also refer to the major leukocyte types that have various roles in the immune system, including activation and deactivation of other immune cells. The T cell can be any T cell such as a cultured T cell, e.g., a primary T cell, or a T cell derived from a cultured T cell line, e.g., a Jurkat, SupT1, etc., or a T cell obtained from a mammal. T cells include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. The T cell can be a CD3⁺ cell. T cells can be CD4⁺, CD8⁺, or CD4⁺ and CD8⁺. The T cell can be any type of T cell, CD4⁺/CD8⁺ double positive T cells, CD4⁺ helper T cells (e.g., T_{H}1 and T_{H}2 cells), CD8⁺ T cells (e.g., cytotoxic T cells), peripheral blood mononuclear cells (PBMC), peripheral blood leukocytes (PBL), tumor infiltrating lymphocytes (TIL), memory T cells, naive T cells, regulatory T cells, γδ T cells, etc. It can be any T cell at any stage of development. Additional types of helper T cells include T_{H}3 (Treg) cells, T_{H}17 cells, T_{H}9 cells, or T_{FH} cells. Additional types of memory T cells include cells such as central memory T cells (T_{CM} cells), stem cell memory T cells (T_{SCM} cells), effector memory T cells (T_{EM} cells and T_{EMRA} cells). A T cell can also refer to a genetically modified T cell, such as a T cell that has been modified to express a T cell receptor (TCR) or a chimeric antigen receptor (CAR). T cells can also be differentiated from stem cells or progenitor cells.

“CD4⁺ T cells” refers to a subset of T cells that express CD4 on their surface and are associated with a cellular immune response. CD4⁺ T cells are characterized by a post-stimulation secretion profile that can include secretion of cytokines such as IFN-γ, TNF-α, IL-2, IL-4 and IL-10. “CD4” is a 55 kD glycoprotein originally defined as a differentiation antigen on T lymphocytes, but was also found on other cells including monocytes/macrophages. The CD4 antigen is a member of the immunoglobulin superfamily and has been implicated as an associative recognition element in MHC (major histocompatibility complex) class II restricted immune responses. On T lymphocytes, the CD4 antigen defines a helper/inducer subset.

“CD8⁺ T cells” refers to a subset of T cells that express CD8 on their surface, are MHC class I restricted, and function as cytotoxic T cells. The “CD8” molecule is a differentiation antigen present on thymocytes, as well as on cytotoxic and suppressor T lymphocytes. The CD8 antigen is a member of the immunoglobulin superfamily and is an associative recognition element in major histocompatibility complex class I restriction interactions.

As used herein, the term “primary” in the context of a primary cell or primary stem cell refers to a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized, e.g., directly without culturing or sub-culturing. In some cases, the primary cells are stimulated, activated, or differentiated. For example, primary T cells can be activated by contact with (e.g., culturing in the presence of) CD3, CD28 agonists, IL-2, IFNγ, or a combination thereof.

As used herein, the term “exogenous” in the context of an element in a cell refers to an element, e.g., a molecule or activity, that has been introduced into a host cell and is not native to that cell. The molecule can be introduced, for example, by introduction of the encoding nucleic acid into host genetic material, such as by integration into a host chromosome, or as non-chromosomal genetic material, such as a plasmid. Thus, the term, when used in connection with expression of an encoding nucleic acid, refers to the introduction of the encoding nucleic acid into a cell in an expressible form. The term “endogenous” refers to a molecule or activity that is present in a host cell under natural, unedited conditions. Similarly, the term, when used in connection with expression of the encoding nucleic acid, refers to expression of the encoding nucleic acid that is contained within the cell and not introduced exogenously.

The term “heterologous” in the context of a nucleic acid refers to a nucleic acid or polypeptide sequence or domain which is not native to a flanking sequence, e.g., wherein the heterologous sequence is not found in nature coupled to the nucleic acid or polypeptide sequences occurring at one or both ends.

The term “homologous” in the context of a nucleic acid refers to a nucleic acid or polypeptide sequence or domain which is native to a flanking sequence, e.g., wherein the homologous sequence is found in nature coupled to the nucleic acid or polypeptide sequences occurring at one or both ends.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The term “mammal” as used herein includes both humans and non-humans and include but is not limited to humans, non-human primates, canines, felines, murines, bovines, equines, and porcines.

The terms “modulate” and “modulation” refer to reducing or inhibiting or, alternatively, activating or increasing, a recited variable.

The terms “protein,” “polypeptide,” and “peptide” are used herein interchangeably.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

As used herein, the term “subject” refers to a human subject. In some embodiments the subject has a disease or condition that can be treated with an engineered cell provided herein or population thereof. In some aspects, the disease or condition is a cancer.

Certain amino acids of a protein can be modified post-transcriptionally and the amino acid sequences provided herein include amino acids that contain a post-translational modification, e.g., deamidation, glycosylation, formation of pyroglutamate, and deletion of C-terminal lysine or other amino acids. For heavy or light chains or their VH or VL domains disclosed herein that have an N-terminal glutamine (Q) or glutamic acid/glutamate (E), the N-terminal Q or E can be replaced by a pyro-glutamate. Accordingly, any VH or VL amino acid sequence disclosed herein having a Q or an E as N-terminal amino acid sequence should be understood to encompass those in which the Q or E is replaced by a pyro-glutamate. Also provided are compositions comprising proteins comprising an N-terminal VH or VL having an N-terminal Q or E, wherein at least about 10%, 20%, 30%, 40%, 50%, 60%, 70%, 80%, 90% or 95% of the proteins in the composition have a pyro-glutamate at the N-terminal amino acid of the VH and/or VL.

### TMPRSS4 Antigen Binding Domains

In some aspects, provided herein are antigen binding domains (e.g., antibodies or antigen binding fragments thereof) that bind to TMPRSS4. In some aspects, provided herein are means for binding to TMPRSS4. In some embodiments, the means for binding to TMPRSS4 comprises an antibody or antigen-binding fragment provided herein. In some embodiments, a TMPRSS4 antibody or antigen-binding fragment or equivalent thereof comprises means for binding a TMPRSS4 protein, optionally binding a human TMPRSS4 protein in the region(s) of human TMPRSS4 bound by the TMPRSS4 antigen binding domains (e.g., an antibody or antigen binding fragment thereof as described in the Examples below). In some embodiments, the means binds a TMPRSS4 protein. In some embodiments, the means binds a human TMPRSS4 protein (e.g., the TMPRSS4 protein of SEQ ID NO: 960) and related isoforms and orthologs. In some embodiments, the means is a TMPRSS4 antibody or antigen-binding fragment or equivalent thereof (e.g., a full length antibody or a F(ab′)₂ fragment, a Fab fragment, a single chain variable fragment (scFv), and a single domain antibody (sdAb), or a functional fragment thereof) means for binding a TMPRSS4 protein. In some embodiments, the means for binding TMPRSS4 includes the anti-TMPRSS4 antibodies and antigen-binding fragments or equivalents thereof described herein.

Transmembrane protease, serine 4 (TMPRSS4 HGNC: 11878, NCBI Entrez Gene: 56649; UniProtKB/Swiss-Prot: Q9NRS4), otherwise known as Transmembrane Serine Protease 4; Membrane-Type Serine Protease 2 (MT-SP2); Channel-Activating Serine Protease 2 (CAP2); Type II Membrane Serine Protease; or CAPH2, is a 48 kDa transmembrane glycoprotein that belongs to the serine protease family of proteins, a promoter of cancer cell invasion. The canonical isoform encodes a type II single pass transmembrane protein with a 384 amino acid extracellular C-terminal domain. An autocatalytic event has been reported to induce self-cleavage between amino acids 204 and 205, resulting in a 150 amino acid extracellular region.

The amino acid and nucleic acid sequences of TMPRSS4 are provided below in Table 3, as well as the amino acid sequences of a catalytically inactive TMPRSS4 (comprising a D290A mutation) and a truncated TMPRSS4 mutant.

TABLE 3
TMPRSS4 sequences

| Human | MLQDPDSDQPLNSLDVKPLRKPRIPMETFRKVGIPIIIALLSLASIIIVVVLIKVILDKYYFLCGQ |
|---|---|
| TMPRSS4 | PLHFIPRKQLCDGELDCPLGEDEEHCVKSFPEGPAVAVRLSKDRSTLQVLDSATGNWFSACFDNFT |
| SEQ ID NO: | EALAETACRQMGYSSKPTFRAVEIGPDQDLDVVEITENSQELRMRNSSGPCLSGSLVSLHCLACGK |
| 960 | SLKTPRVVGVEEASVDSWPWQVSIQYDKQHVCGGSILDPHWVLTAAHCFRKHTDVFNWKVRAGSDK |
| | LGSFPSLAVAKIIIIEFNPMYPKDNDIALMKLQFPLTFSGTVRPICLPFFDEELTPATPLWIIGWG |
| | FTKQNGGKMSDILLQASVQVIDSTRCNADDAYQGEVTEKMMCAGIPEGGVDTCQGDSGGPLMYQSD |
| | QWHVVGIVSWGYGCGGPSTPGVYTKVSAYLNWIYNVWKAEL |
| Human | ATGCTCCAGGACCCAGACTCCGACCAGCCTCTTAACTCACTTGATGTCAAGCCCTTGCGAAAACCT |
| TMPRSS4 | CGGATTCCAATGGAAACTTTCAGAAAAGTGGGTATCCCGATTATCATCGCGTTGCTCAGCTTGGCC |
| SEQ ID NO: | TCAATAATCATTGTGGTCGTATTAATCAAAGTGATCCTGGACAAATACTACTTTCTCTGCGGTCAG |
| 961 | CCCTTGCATTTCATCCCAAGGAAGCAGCTCTGCGATGGGGAGCTGGATTGTCCCTTGGGCGAAGAT |
| | GAGGAACACTGCGTCAAAAGTTTTCCAGAAGGGCCCGCTGTCGCGGTGCGGCTGAGCAAGGATCGC |
| | TCTACCCTGCAAGTGCTGGACTCCGCGACCGGGAACTGGTTCAGTGCTTGTTTTGACAATTTCACC |
| | GAGGCCCTGGCTGAGACAGCCTGCCGCCAGATGGGGTATAGCTCCAAACCAACTTTTAGAGCTGTG |
| | GAGATTGGACCGGACCAGGACCTGGACGTTGTGGAAATCACGGAGAACTCCCAAGAACTCCGAATG |
| | AGAAATTCTAGTGGACCTTGTCTGTCTGGCTCCCTGGTATCTCTACACTGTCTGGCTTGCGGCAAA |
| | TCACTTAAGACACCCAGGGTGGTCGGAGTGGAGGAGGCCTCCGTGGATAGTTGGCCTTGGCAAGTC |
| | TCTATTCAATACGATAAGCAGCACGTGTGCGGAGGTTCGATACTCGATCCGCACTGGGTCTTGACA |
| | GCAGCCCACTGCTTCCGGAAACATACTGATGTTTTTAATTGGAAAGTTCGCGCAGGGTCTGACAAG |
| | CTGGGCTCATTCCCAAGCCTGGCTGTCGCAAAAATCATAATTATCGAGTTCAACCCTATGTACCCG |
| | AAAGACAACGACATCGCCCTTATGAAACTGCAATTTCCGCTAACATTCAGCGGAACCGTCCGACCA |
| | ATTTGCCTGCCCTTTTTCGACGAAGAATTAACACCCGCAACACCTCTGTGGATAATCGGCTGGGGC |
| | TTTACTAAGCAGAACGGCGGCAAGATGAGTGACATACTACTACAAGCAAGTGTGCAAGTTATTGAT |
| | AGTACCCGTTGTAATGCCGATGATGCTTACCAAGGCGAAGTTACGGAGAAGATGATGTGCGCAGGG |
| | ATTCCAGAGGGTGGCGTGGATACCTGTCAGGGTGATAGCGGAGGGCCCTTAATGTATCAGTCCGAC |
| | CAGTGGCATGTCGTGGGGATAGTTAGCTGGGGCTACGGATGTGGAGGCCCTAGCACTCCCGGCGTC |
| | TATACCAAGGTGTCGGCCTATCTTAATTGGATCTATAACGTGTGGAAAGCCGAGTTG |
| TMPRSS4 | MLQDPDSDQPLNSLDVKPLRKPRIPMETFRKVGIPIIIALLSLASIIIVVVLIKVILDKYYFLCGQ |
| D290A- | PLHFIPRKQLCDGELDCPLGEDEEHCVKSFPEGPAVAVRLSKDRSTLQVLDSATGNWFSACFDNFT |
| Catalytically | EALAETACRQMGYSSKPTFRAVEIGPDQDLDVVEITENSQELRMRNSSGPCLSGSLVSLHCLACGK |
| inactive | SLKTPRVVGVEEASVDSWPWQVSIQYDKQHVCGGSILDPHWVLTAAHCFRKHTDVFNWKVRAGSDK |
| SEQ ID NO: | LGSFPSLAVAKIIIIEFNPMYPKDNAIALMKLQFPLTFSGTVRPICLPFFDEELTPATPLWIIGWG |
| 996 | FTKQNGGKMSDILLQASVQVIDSTRCNADDAYQGEVTEKMMCAGIPEGGVDTCQGDSGGPLMYQSD |
| | QWHVVGIVSWGYGCGGPSTPGVYTKVSAYLNWIYNVWKAEL |
| TMPRSS4_ | MLQDPDSDQPLNSLDVKPLRKPRIPMETFRKVGIPIIIALLSLASIIIVVVLIKVILDKYYFLCGQ |
| Truncated | PLHFIPRKQLCDGELDCPLGEDEEHCVKSFPEGPAVAVRLSKDRSTLQVLDSATGNWFSACFDNFT |
| mutant 1- | EALAETACRQMGYSSKPTFRAVEIGPDQDLDVVEITENSQELRMRNSSGPCLSGSLVSLHCLACGK |
| 204 | SLKTPR |
| SEQ ID NO: | |
| 997 | |

In some embodiments, the TMPRSS4 antigen-binding moiety (e.g., an antigen binding protein or domain such as an antibody of antigen binding fragment thereof) is selected from the group consisting of an antibody, a nanobody, a diabody, a triabody, or a minibody, a F(ab′)₂ fragment, a Fab fragment, a single chain variable fragment (scFv), and a single domain antibody (sdAb), or a functional fragment thereof. In some embodiments, the antigen-binding moiety comprises an scFv. The antigen-binding moiety can include naturally-occurring amino acid sequences or can be engineered, designed, or modified so as to provide desired and/or improved properties, e.g., increased binding affinity.

Table 4 provides exemplary amino acid sequences of antibody heavy chain variable domains (VHs) and light chain variable domains (VLs) that, in combination, bind to TMPRSS4 with CDR-H1, CDR-H2, CDR-H3, CDR-L1, CDR-L2, and CDR-L3 sequences noted below the respective VH or VL sequence. The CDR sequences provided in Table 4 are annotated using the Kabat scheme.

TABLE 4
TMPRSS4 VH and VL Amino Acid Sequences

| Clone | VH Sequence | VL Sequence |
|---|---|---|
| TMPRSS4 | QVQLVQSGAEVKKPGSSVKVSCKASGYTFTD | DIQMTQSPSSLSASVGDRVTITCQASQDISNY |
| Ab1 | YYMHWVRQAPGQGLEWMGGIIPIFGTANYAQ | LNWYQQKPGKAPKLLIYKASSLESGVPSRFSG |
| | KFQGRVTITADESTSTAYMELSSLRSEDTAV | SGSGTDFTLTISSLQPEDFATYYCQQSSRIPP |
| | YYCAKEGANGYWGQGTLVTVSS (SEQ ID | TFGQGTKVEIK (SEQ ID NO: 2) |
| | NO: 1) | QASQDISNYLN (SEQ ID NO: 6) |
| | DYYMH (SEQ ID NO: 3) | KASSLES (SEQ ID NO: 7) |
| | GIIPIFGTANYAQKFQG (SEQ ID NO: 4) | QQSSRIPPT (SEQ ID NO: 8) |
| | EGANGY (SEQ ID NO: 5) | |
| TMPRSS4 | EVQLLESGGGLVQPGGSLRLSCAASGFTFSD | DIQMTQSPSSLSASVGDRVTITCRASQSTNNY |
| Ab2 | YYMSWVRQAPGKGLEWVSYISGSGDAIYYAD | VNWYQQKPGKAPKLLIYAASSLQSGVPSRFSG |
| | SVKGRFTISRDNSKNTLYLQMNSLRAEDTAV | SGSGTDFTLTISSLQPEDFATYYCQQSYSIPL |
| | YYCARDRSDCGGDDRFLCDGYFDLWGRGTLV | TFGPGTKVDIK (SEQ ID NO: 10) |
| | SLS (SEQ ID NO: 9) | RASQSTNNYVN (SEQ ID NO: 14) |
| | DYYMS (SEQ ID NO: 11) | AASSLQS (SEQ ID NO: 15) |
| | YISGSGDAIYYADSVKG(SEQ ID NO: 12) | QQSYSIPLT (SEQ ID NO: 16) |
| | DRSDCGGDDRFLCDGYFDL(SEQ ID NO: 13) | |
| TMPRSS4 | QVQLVQSGAEVKKPGSSVKVSCKASGYTFTS | DIVMTQSPLSLPVTPGEPASISCRSSGSLLHS |
| Ab3 | YDINWVRQAPGQGLEWMGGIIPIFGTTKFAQ | NGYNYLDWYLQKPGQSPQLLIYAASSLQSGVP |
| | KFQGRVTITADESTSTAYMELSSLRSEDTAV | DRFSGSGSGTDFTLKISRVEAEDVGVYYCMQG |
| | YYCARDWYSSSWYNGDRGDWEDPWGQGTLVT | THWPGTFGQGTKVEIK (SEQ ID NO: 18) |
| | VSS (SEQ ID NO: 17) | RSSGSLLHSNGYNYLD (SEQ ID NO: 22) |
| | SYDIN (SEQ ID NO: 19) | AASSLQS (SEQ ID NO: 15) |
| | GIIPIFGTTKFAQKFQG (SEQ ID NO: 20) | MQGTHWPGT (SEQ ID NO: 23) |
| | DWYSSSWYNGDRGDWFDP (SEQ ID NO: 21) | |
| TMPRSS4 | QVQLVQSGAEVKKPGASVKVSCKASGYFFTT | DIVMTQSPLSLPVTPGEPASISCRSSQSLLHS |
| Ab4 | YYLHWVRQAPGQGLEWMGVINPNSRLTSYAE | NGYNYLDWYLQKPGQSPQLLIYAASTLQSGVP |
| | SFQGRVTMTRDTSTSTVYMELSSLRSEDTAV | DRFSGSGSGTDFTLKISRVEAEDVGVYYCMQG |
| | YYCEREMFPSSYGIDVWGQGTTVTVSS (SEQ | THWPPTFGQGTKLEIK (SEQ ID NO: 25) |
| | ID NO: 24) | RSSQSLLHSNGYNYLD (SEQ ID NO: 29) |
| | TYYLH (SEQ ID NO: 26) | AASTLQS (SEQ ID NO: 30) |
| | VINPNSRLTSYAESFQG (SEQ ID NO: 27) | MQGTHWPPT (SEQ ID NO: 31) |
| | EMFPSSYGIDV (SEQ ID NO: 28) | |
| TMPRSS4 | QVQLVQSGAEVKKPGSSVKVSCKASGYTFTS | EIVMTQSPATLSVSPGERATLSCRASQSVSSN |
| Ab5 | YYMHWVRQAPGQGLEWMGRIIPILGATDYAQ | LAWYQQKPGQAPRLLIYGASTRATGIPARFSG |
| | KFQGRVTITADESTSTAYMELSSLRSEDTAV | SGSGTEFTLTISSLQSEDFAVYYCQQYYSPFP |
| | YYCARAGYSSIAARPAFWGQGTLVTVSS | LTFGGGTKVEIK (SEQ ID NO: 33) |
| | (SEQ ID NO: 32) | RASQSVSSNLA (SEQ ID NO: 37) |
| | SYYMH (SEQ ID NO: 34) | GASTRAT (SEQ ID NO: 38) |
| | RIIPILGATDYAQKFQG (SEQ ID NO: 35) | QQYYSPFPLT (SEQ ID NO: 39) |
| | AGYSSIAARPAF (SEQ ID NO: 36) | |
| TMPRSS4 | QVQLVQSGAEVKKPGASVKVSCKASGYTFTS | DIVMTQSPDSLAVSLGERATINCKSSQSVLYS |
| Ab6 | YYMHWVRQAPGQGLEWLGIINPSDYTTSYAQ | SNNKNYLAWYQQKPGQPPKLLIYWASTRESGV |
| | KFQGRVTMTRDTSTSTVYMELSSLRSEDTAV | PDRFSGSGSGTDFTLTISSLQAEDVAIYYCQQ |
| | YYCARVASSSWYPGDENWYFDLWGRGTLVTV | YYAIPWTFGQGTKVEIK (SEQ ID NO: 41) |
| | SS (SEQ ID NO: 40) | KSSQSVLYSSNNKNYLA (SEQ ID NO: 44) |
| | SYYMH (SEQ ID NO: 34) | WASTRES (SEQ ID NO: 45) |
| | IINPSDYTTSYAQKFQG (SEQ ID NO: 42) | QQYYAIPWT (SEQ ID NO: 46) |
| | VASSSWYPGDENWYFDL (SEQ ID NO: 43) | |
| TMPRSS4 | QVQLVQSGAEVKKPGASVKVSCKASGYTFSR | EIVMTQSPATLSVSPGERATLSCRASQRVSNN |
| Ab7 | YFMHWVRQAPGQGLEWVGWINPNSGNTGYAQ | YLAWYQQKPGQAPRLLIYGASTRASGIPARFS |
| | KFQGRVTMTRDTSTSTVYMELSSLRSEDTAV | GSGSGTEFTLTISSLQSEDFAVYYCQQYGSTP |
| | YYCARVVTGGRLDVWGQGTTVTVSS (SEQ ID | YTFGQGTKVEIK (SEQ ID NO: 48) |
| | NO: 47) | RASQRVSNNYLA (SEQ ID NO: 52) |
| | RYFMH (SEQ ID NO: 49) | GASTRAS (SEQ ID NO: 53) |
| | WINPNSGNTGYAQKFQG (SEQ ID NO: 50) | QQYGSTPYT (SEQ ID NO: 54) |
| | VVTGGRLDV (SEQ ID NO: 51) | |
| TMPRSS4 | QVQLVQSGAEVKKPGASVKVSCKASGYRFTS | DIQMTQSPSSLSASVGDRVTITCRASQSISSW |
| Ab8 | QYMHWVRQAPGQGLEWMGIINPSGGSTSYAQ | LAWYQQKPGKAPKLLIYGASSLQSGVPSRFSG |
| | KFQGRVTMTRDTSTSTVYMELSSLRSEDTAV | SGSGTDFTLTISSLQPEDFATYYCQQANSFPP |
| | YYCARGRIAVAGHPLGYWGQGTLVTVSS | TFGGGTKVEIK (SEQ ID NO: 56) |
| | (SEQ ID NO: 55) | RASQSISSWLA (SEQ ID NO: 60) |
| | SQYMH (SEQ ID NO: 57) | GASSLQS (SEQ ID NO: 61) |
| | IINPSGGSTSYAQKFQG (SEQ ID NO: 58) | QQANSFPPT (SEQ ID NO: 62) |
| | GRIAVAGHPLGY (SEQ ID NO: 59) | |
| TMPRSS4 | QVQLVQSGAEVKKPGASVKVSCKASGYTFTR | DIQMTQSPSSLSASVGDRVTITCQASQDISRF |
| Ab9 | YYMHWVRQAPGQGLEWMGWINPNSGGTNYAQ | LHWYQQKPGKAPKLLIYGASNLKSGVPSRFSG |
| | KFQGRVTMTRDTSTSTVYMELSSLRSEDTAV | SGSGTDFTLTISSLQPEDFATYYCQQSYSTPP |
| | YYCARGGSWGSGPLGYWGQGTLVTVSS (SEQ | TFGGGTKVEIK (SEQ ID NO: 64) |
| | ID NO: 63) | QASQDISRFLH (SEQ ID NO: 68) |
| | RYYMH (SEQ ID NO: 65) | GASNLKS (SEQ ID NO: 69) |
| | WINPNSGGTNYAQKFQG (SEQ ID NO: 66) | QQSYSTPPT (SEQ ID NO: 70) |
| | GGSWGSGPLGY (SEQ ID NO: 67) | |
| TMPRSS4 | QVQLVQSGAEVKKPGASVKVSCKASGYTFTS | EIVMTQSPATLSVSPGERATLSCRASQSVSSY |
| Ab10 | YYMHWVRQAPGQGLEWMGIINPSGAGTTYGH | LAWYQQKPGQAPRLLIYGASTRATGIPARFSG |
| | NFQGRVTMTRDTSTSTVYMELSSLRSEDTAV | SGSGTEFTLTISSLQSEDFAVYYCQQYGSSPG |
| | YYCARGPRDTAMVRFDYWGQGTLVTVSS | TFGQGTKLEIK (SEQ ID NO: 72) |
| | (SEQ ID NO: 71) | RASQSVSSYLA (SEQ ID NO: 75) |
| | SYYMH (SEQ ID NO: 34) | GASTRAT (SEQ ID NO: 38) |
| | IINPSGAGTTYGHNFQG (SEQ ID NO: 73) | QQYGSSPGT (SEQ ID NO: 76) |
| | GPRDTAMVRFDY (SEQ ID NO: 74) | |
| TMPRSS4 | QVQLVQSGAEVKKPGASVKVSCKASGGTFSN | DIQMTQSPSSLSASVGDRVTITCRASQSINNY |
| Ab11 | YAISWVRQAPGQGLEWVGRINPNSGGTNYAQ | LNWYQQKPGKAPKLLIYAASSLQSGVPSRFSG |
| | KFQGRVTMTRDTSTSTVYMELSSLRSEDTAV | SGSGTDFTLTISSLQPEDFATYYCQQSYSTKW |
| | YYCARGRYSSSSWGQGTLVTVSS (SEQ ID | TFGQGTKVEIK (SEQ ID NO: 78) |
| | NO: 77) | RASQSINNYLN (SEQ ID NO: 82) |
| | NYAIS (SEQ ID NO: 79) | AASSLQS (SEQ ID NO: 15) |
| | RINPNSGGTNYAQKFQG (SEQ ID NO: 80) | QQSYSTKWT (SEQ ID NO: 83) |
| | GRYSSSS (SEQ ID NO: 81) | |
| TMPRSS4 | QVQLVQSGAEVKKPGASVKVSCKASGYTFSN | DIQMTQSPSSLSASVGDRVTITCRASQGISRW |
| Ab12 | YYIHWVRQAPGQGLEWMGWINPNSGDTNYAQ | LAWYQQKPGKAPKLLIYGASNLQTGVPSRFSG |
| | KFQGRVTMTRDTSTSTVYMELSSLRSEDTAV | SGSGTDFTLTISSLQPEDFATYYCQQSYSTPP |
| | YYCARGMTWRTSAATYWGQGTLVTVSS (SEQ | TFGPGTKVDIK (SEQ ID NO: 85) |
| | ID NO: 84) | RASQGISRWLA (SEQ ID NO: 89) |
| | NYYIH (SEQ ID NO: 86) | GASNLQT (SEQ ID NO: 90) |
| | WINPNSGDTNYAQKFQG (SEQ ID NO: 87) | QQSYSTPPT (SEQ ID NO: 70) |
| | GMTWRTSAATY (SEQ ID NO: 88) | |
| TMPRSS4 | QVQLVQSGAEVKKPGASVKVSCKASGYTFTN | EIVMTQSPATLSVSPGERATLSCRASQSVNGN |
| Ab13 | YYMHWVRQAPGQGLEWMGWISAYNGNTNYAQ | YLAWYQQKPGQAPRLLIYGVSSRASGIPARFS |
| | KLQGRVTMTRDTSTSTVYMELSSLRSEDTAV | GSGSGTEFTLTISSLQSEDFAVYYCQQYGSSP |
| | YYCATASGWGHSNSAGYWGQGTLVTVSS | YTFGQGTKVEIK (SEQ ID NO: 92) |
| | (SEQ ID NO: 91) | RASQSVNGNYLA (SEQ ID NO: 96) |
| | NYYMH (SEQ ID NO: 93) | GVSSRAS (SEQ ID NO: 97) |
| | WISAYNGNTNYAQKLQG (SEQ ID NO: 94) | QQYGSSPYT (SEQ ID NO: 98) |
| | ASGWGHSNSAGY (SEQ ID NO: 95) | |
| TMPRSS4 | EVQLLESGGGLVQPGGSLRLSCAASGFTFSN | DIQMTQSPSSLSASVGDRVTITCRASQRISTY |
| Ab14 | HYMSWVRQAPGKGLEWVSAISGSGGSTYYAD | LNWYQQKPGKAPKLLIYSASTLQAGVPSRFSG |
| | SVKGRFTISRDNSKNTLYLQMNSLRAGDTAV | SGSGTDFTLTISSLQPEDFATYYCQQAYSLPW |
| | YYCARDRYRWGRGYFQHWGQGTLVTVSS | TFGQGTKLEIK (SEQ ID NO: 100) |
| | (SEQ ID NO: 99) | RASQRISTYLN (SEQ ID NO: 104) |
| | NHYMS (SEQ ID NO: 101) | SASTLQA (SEQ ID NO: 105) |
| | AISGSGGSTYYADSVKG (SEQ ID NO: 102) | QQAYSLPWT (SEQ ID NO: 106) |
| | DRYRWGRGYFQH (SEQ ID NO: 103) | |
| TMPRSS4 | QVQLVQSGAEVKKPGASVKVSCKASGYTFTR | DIQMTQSPSSLSASVGDRVTITCRASQSINTW |
| Ab15 | YYMHWVRQAPGQGLEWMGWINPNSGVTNFAQ | LAWYQQKPGKAPKLLIYAASSLQSGVPSRFSG |
| | KFQGRVTMTRDTSTSTVYMELSSLRSEDTAV | SGSGTDFTLTISSLQPEDFATYYCQQAISFPL |
| | YYCARVRIGWLQSPPLYWGQGTLVTVSS | TFGGGTKVEIK (SEQ ID NO: 108) |
| | (SEQ ID NO: 107) | RASQSINTWLA (SEQ ID NO: 111) |
| | RYYMH (SEQ ID NO: 65) | AASSLQS (SEQ ID NO: 15) |
| | WINPNSGVTNFAQKFQG (SEQ ID NO: 109) | QQAISFPLT (SEQ ID NO: 112) |
| | VRIGWLQSPPLY (SEQ ID NO: 110) | |
| TMPRSS4 | QVQLVQSGAEVKKPGASVKVSCKASGYTFSR | DIQMTQSPSSLSASVGDRVTITCRASQSINRW |
| Ab16 | HYMHWVRQAPGQGLEWMGRINPNSGGTNYAQ | LAWYQQKPGKAPKLLIYGASNLQSGVPSRFSG |
| | KFQGRVTMTRDTSTSTVYMELSSLRSEDTAV | SGSGTDFTLTISSLQPEDFATYYCQQANSFPY |
| | YYCARSIYGDYWFDPWGQGTLVTVSS (SEQ | TFGQGTKLEIK (SEQ ID NO: 114) |
| | ID NO: 113) | RASQSINRWLA (SEQ ID NO: 117) |
| | RHYMH (SEQ ID NO: 115) | GASNLQS (SEQ ID NO: 118) |
| | RINPNSGGTNYAQKFQG (SEQ ID NO: 80) | QQANSFPYT (SEQ ID NO: 119) |
| | SIYGDYWEDP (SEQ ID NO: 116) | |
| TMPRSS4 | QVQLVQSGAEVKKPGASVKVSCKASGYTFTS | DIQMTQSPSSLSASVGDRVTITCRASQGISSY |
| Ab17 | YYIHWVRQAPGQGLEWMGWMSPNSGDTGYAQ | LNWYQQKPGKAPKLLIYAASRLQSGVPSRFSG |
| | KFQGRVTMTRDTSTSTVYMELSSLRSEDTAV | SGSGTDFTLTISSLQPEDFATYYCQQSYRSPP |
| | YYCARLVRGGFDYWGQGTLVTVSS (SEQ ID | TFGQGTKLEIK (SEQ ID NO: 121) |
| | NO: 120) | RASQGISSYLN (SEQ ID NO: 125) |
| | SYYIH (SEQ ID NO: 122) | AASRLQS (SEQ ID NO: 126) |
| | WMSPNSGDTGYAQKFQG (SEQ ID NO: 123) | QQSYRSPPT (SEQ ID NO: 127) |
| | LVRGGFDY (SEQ ID NO: 124) | |
| TMPRSS4 | QVQLVQSGAEVKKPGASVKVSCKASGYTFTS | DIVMTQSPDSLAVSLGERATINCKSSQSVLYS |
| Ab18 | SGINWVRQAPGQGLEWMGWINPNSGGAKYAQ | SNNKNYLAWYQQKPGQPPKLLIYWASTRESGV |
| | RFQGRVTMTRDTSTSTVYMELSSLRSEDTAV | PDRFSGSGSGTDFTLTISSLQAEDVAVYHCQQ |
| | YYCARARGYSGSKRDFQHWGQGTLVTVSS | YYNTPFTFGPGTKVDIK (SEQ ID NO: 129) |
| | (SEQ ID NO: 128) | KSSQSVLYSSNNKNYLA (SEQ ID NO: 44) |
| | SSGIN (SEQ ID NO: 130) | WASTRES (SEQ ID NO: 45) |
| | WINPNSGGAKYAQRFQG (SEQ ID NO: 131) | QQYYNTPFT (SEQ ID NO: 133) |
| | ARGYSGSKRDFQH (SEQ ID NO: 132) | |
| TMPRSS4 | QVQLVQSGAEVKKPGASVKVSCKASGYTENR | DIQMTQSPSSLSASVGDRVTITCRASQSISRY |
| Ab19 | KFMHWVRQAPGQGLEWMGWMNPNNGATNYAQ | LNWYQQKPGKAPKLLIYGASNLQSGVPSRFSG |
| | KFQGRVTMTRDTSTSTVYMELSSLRSEDTAV | SGSGTDFTLTISSLQPEDFATYYCQQSYSTPP |
| | YYCARGRGYYGSGSYYGDYWGQGTLVTVSS | TFGPGTKVDIK (SEQ ID NO: 135) |
| | (SEQ ID NO: 134) | RASQSISRYLN (SEQ ID NO: 139) |
| | RKFMH (SEQ ID NO: 136) | GASNLQS (SEQ ID NO: 118) |
| | WMNPNNGATNYAQKFQG (SEQ ID NO: 137) | QQSYSTPPT (SEQ ID NO: 70) |
| | GRGYYGSGSYYGDY (SEQ ID NO: 138) | |
| TMPRSS4 | QVQLVQSGAEVKKPGASVKVSCKASGYTFTR | DIQMTQSPSSLSASVGDRVTITCRASQNIATY |
| Ab20 | YYMHWVRQAPGQGLEWMGWMNPNSGNAGYAQ | LSWYQQKPGKAPKLLIYGASALRSGVPSRFSG |
| | KLQGRVTMTRDTSTSTVYMELSSLRSEDTAV | SGSGTDFTLTISSLQPEDFATYYCLQHNTYPL |
| | YYCARGYNWFDPWGQGTLVTVSS (SEQ ID | TFGGGTKVEIK (SEQ ID NO: 141) |
| | NO: 140) | RASQNIATYLS (SEQ ID NO: 144) |
| | RYYMH (SEQ ID NO: 65) | GASALRS (SEQ ID NO: 145) |
| | WMNPNSGNAGYAQKLQG (SEQ ID NO: 142) | LQHNTYPLT (SEQ ID NO: 146) |
| | GYNWFDP (SEQ ID NO: 143) | |
| TMPRSS4 | QVQLVQSGAEVKKPGASVKVSCKASGYSFSG | DIQMTQSPSSLSASVGDRVTITCRASQSISRY |
| Ab21 | YYLHWVRQAPGQGLEWMGWMNPDSGNTGYAQ | LNWYQQKPGKAPKLLIYAASTLQSGVPSRFSG |
| | NFQGRVTMTRDTSTSTVYMELSSLRSEDTAV | SGSGTDFTLTISSLQPEDFATYYCQQSYSTPV |
| | YYCARLHRGGHDYWGQGTLVTVSS (SEQ ID | TFGQGTRLEIK (SEQ ID NO: 148) |
| | NO: 147) | RASQSISRYLN (SEQ ID NO: 139) |
| | GYYLH (SEQ ID NO: 149) | AASTLQS (SEQ ID NO: 30) |
| | WMNPDSGNTGYAQNFQG (SEQ ID NO: 150) | QQSYSTPVT (SEQ ID NO: 152) |
| | LHRGGHDY (SEQ ID NO: 151) | |
| TMPRSS4 | QVQLVQSGAEVKKPGASVKVSCKASGYTFTS | DIQMTQSPSSLSASVGDRVTITCRAGQNIKRY |
| Ab22 | YYMHWVRQAPGQGLEWMGRINPHSGDADFVD | LNWYQQKPGKAPKLLIYAASSLQSGVPSRFSG |
| | KFQGRVTMTRDTSTSTVYMELSSLRSEDTAV | SGSGTDFTLTISSLQPEDFATYYCQQSYSSPL |
| | YYCARDRRGYGGNSLDYWGQGTLVTVSS | TFGGGTKVEIK (SEQ ID NO: 154) |
| | (SEQ ID NO: 153) | RAGQNIKRYLN (SEQ ID NO: 157) |
| | SYYMH (SEQ ID NO: 34) | AASSLQS (SEQ ID NO: 15) |
| | RINPHSGDADFVDKFQG (SEQ ID NO: 155) | QQSYSSPLT (SEQ ID NO: 158) |
| | DRRGYGGNSLDY (SEQ ID NO: 156) | |
| TMPRSS4 | QVQLVQSGAEVKKPGASVKVSCKASGYTFTR | EIVMTQSPATLSVSPGERATLSCRASQSVGNY |
| Ab23 | NYLHWVRQAPGQGLEWMGIINPSGGSTTYAQ | LAWYQQKPGQAPRLLIYGASTRATGIPARFSG |
| | KFQGRVTMTRDTSTSTVYMELSSLRSEDTAV | SGSGTEFTLTISSLQSEDFAVYYCQQYHSSPP |
| | YYCARGRTWFRSGMDVWGQGTTVTVSS (SEQ | YTFGQGTKVEIK (SEQ ID NO: 160) |
| | ID NO: 159) | RASQSVGNYLA (SEQ ID NO: 164) |
| | RNYLH (SEQ ID NO: 161) | GASTRAT (SEQ ID NO: 38) |
| | IINPSGGSTTYAQKFQG (SEQ ID NO: 162) | QQYHSSPPYT (SEQ ID NO: 165) |
| | GRTWFRSGMDV (SEQ ID NO: 163) | |
| TMPRSS4 | QVQLVQSGAEVKKSGASVKVSCKASGYTFTS | DIQMTQSPSSLSASVGDRVTITCRASQSISSW |
| Ab24 | YYMHWVRQAPGQGLEWMGVINPSGGTTSYAQ | LAWYQQKPGKAPKLLIYAASTLQSGVPSRFSG |
| | KFQGRVTMTRETSTSTVYMELSSLRSEDTAV | SGSGTDFTLTISSLQPEDFATYYCQQSYNTPY |
| | YYCARGRGWLRSALGYWGQGTLVTVSS (SEQ | TFGQGTKLEIK (SEQ ID NO: 167) |
| | ID NO: 166) | RASQSISSWLA (SEQ ID NO: 60) |
| | SYYMH (SEQ ID NO: 34) | AASTLQS (SEQ ID NO: 30) |
| | VINPSGGTTSYAQKFQG (SEQ ID NO: 168) | QQSYNTPYT (SEQ ID NO: 170) |
| | GRGWLRSALGY (SEQ ID NO: 169) | |
| TMPRSS4 | QVQLVQSGAEVKKPGASVKVSCKASGGRFST | EIVMTQSPATLSVSPGERATLSCRASQSVSSN |
| Ab25 | YALSWVRQAPGQGLEWMGWINPNSGGTNYAQ | YLAWYQQKPGQAPRLLIYGISTRASGIPARFS |
| | KFQGRVTMTRDTSTSTVYMELSSLRSEDTAV | GSGSGTEFTLTISSLQSEDFAVYYCQQRSNWP |
| | YYCAKSLWWSPSHYYYYGMDVWGQGTTVTVS | PSITFGQGTRLEIK (SEQ ID NO: 172) |
| | S (SEQ ID NO: 171) | RASQSVSSNYLA (SEQ ID NO: 175) |
| | TYALS (SEQ ID NO: 173) | GISTRAS (SEQ ID NO: 176) |
| | WINPNSGGTNYAQKFQG (SEQ ID NO: 66) | QQRSNWPPSIT (SEQ ID NO: 177) |
| | SLWWSPSHYYYYGMDV (SEQ ID NO: 174) | |
| TMPRSS4 | EVQLLESGGGLVQPGGSLRLSCAASGFTFSS | DIQMTQSPSSLSASVGDRVTITCRASQNVGSW |
| Ab26 | YAMHWVRQAPGKGLEWVAVIWYDGSSKYYAD | LAWYQQKPGKAPKLLIYAASSLQSGVPSRFSG |
| | SVKGRFTISRDNSKNTLYLQMNSLRAEDTAV | SGSGTDFTLTIRSLQPEDFATYYCQQSYSTPI |
| | YYCARGEVRRGFQHWGQGTLVTVSS (SEQ ID | TFGQGTRLEIK (SEQ ID NO: 179) |
| | NO: 178) | RASQNVGSWLA (SEQ ID NO: 183) |
| | SYAMH (SEQ ID NO: 180) | AASSLQS (SEQ ID NO: 15) |
| | VIWYDGSSKYYADSVKG (SEQ ID NO: 181) | QQSYSTPIT (SEQ ID NO: 184) |
| | GEVRRGFQH (SEQ ID NO: 182) | |
| TMPRSS4 | QVQLVQSGAEVKKPGASVKVSCKASGYTFSR | DIQMTQSPSSLSASVGDRVTITCRASQSISTW |
| Ab27 | YYMHWVRQAPGQGLEWMGWMNPNSGDTGYAQ | LAWYQQKPGKAPKLLIYAASSLQSGVPSRFSG |
| | KFQGRVTMTRDTSTSTVYMELSSLRSEDTAV | SGSGTDFTLTISSLQPEDFATYYCQQLSSYPL |
| | YYCAKGREWLRSPFDYWGQGTLVTVSS (SEQ | TFGQGTKVEIK (SEQ ID NO: 186) |
| | ID NO: 185) | RASQSISTWLA (SEQ ID NO: 292) |
| | RYYMH (SEQ ID NO: 187) | AASSLQS (SEQ ID NO: 15) |
| | WMNPNSGDTGYAQKFQG (SEQ ID NO: 188) | QQLSSYPLT (SEQ ID NO: 190) |
| | GREWLRSPFDY (SEQ ID NO: 189) | |
| TMPRSS4 | QVQLVQSGAEVKKPGASVKVSCKASGYTFTG | DIQMTQSPSSLSASVGDRVTITCRASQGIGNY |
| Ab28 | YYMHWVRQAPGQGLEWMGWMNPNSGNTGYAQ | LAWYQQKPGKAPKLLIYAASSLESGVPSRFSG |
| | KFQGRVTMTRDTSTSTVYMELSSLRSEDTAV | SGSGTDFTLTISSLQPEDFATYYCQQGYRFPP |
| | YYCARLRAKGGGFDYWGQGTLVTVSS (SEQ | TFGPGTKVDIK (SEQ ID NO: 192) |
| | ID NO: 191) | RASQGIGNYLA (SEQ ID NO: 196) |
| | GYYMH (SEQ ID NO: 193) | AASSLES (SEQ ID NO: 197) |
| | WMNPNSGNTGYAQKFQG (SEQ ID NO: 194) | QQGYRFPPT (SEQ ID NO: 198) |
| | LRAKGGGFDY (SEQ ID NO: 195) | |
| TMPRSS4 | QVQLVQSGAEVKKPGASVKVSCKASGYTFAN | DIVMTQSPLSLPVTPGEPASISCRSSQSLLHS |
| Ab29 | YNIHWVRQAPGQGLEWMGWMNPNSGNTGYAQ | NGYNYLDWYLQKPGQSPQLLIYLGSNRASGVP |
| | KFQGRVTMTRDTSTSTVYMELSSLRSEDTAV | DRFSGSGSGTDFTLKISRVEAEDVGVYYCMQS |
| | YYCARPRYSSGWYGWYFDLWGRGTLVTVSS | TYWPPTFGQGTKLEIK (SEQ ID NO: 200) |
| | (SEQ ID NO: 199) | RSSQSLLHSNGYNYLD (SEQ ID NO: 29) |
| | NYNIH (SEQ ID NO: 201) | LGSNRAS (SEQ ID NO: 203) |
| | WMNPNSGNTGYAQKFQG (SEQ ID NO: 194) | MQSTYWPPT (SEQ ID NO: 204) |
| | PRYSSGWYGWYFDL (SEQ ID NO: 202) | |
| TMPRSS4 | QVQLVQSGAEVKKPGASVKVSCKASGYTFTT | EIVMTQSPATLSVSPGERATLSCRASQSVGRY |
| Ab30 | YYMHWVRQAPGQGLEWMGWMNPNSGNTGYAQ | LAWYQQKPGQAPRLLIYGASTRATGIPARFSG |
| | KFQGRVTMTRDTSTSTVYMELSSLRSEDTAV | SGSGTEFTLTISSLQSEDFAVYYCQHYDSSPM |
| | YYCARARTWLLSPFDYWGQGTLVTVSS (SEQ | YTFGQGTKLEIK (SEQ ID NO: 206) |
| | ID NO: 205) | RASQSVGRYLA (SEQ ID NO: 209) |
| | TYYMH (SEQ ID NO: 207) | GASTRAT (SEQ ID NO: 38) |
| | WMNPNSGNTGYAQKFQG (SEQ ID NO: 194) | QHYDSSPMYT (SEQ ID NO: 210) |
| | ARTWLLSPEDY (SEQ ID NO: 208) | |
| TMPRSS4 | QVQLVQSGAEVKKPGSSVKVSCKASGYTFRG | EIVMTQSPATLSVSPGERATLSCRASQSVRSY |
| Ab31 | SGISWVRQAPGQGLEWMGIIYPADSETRYSP | LAWYQQKPGQAPRLLIYGASTRATGIPARFSG |
| | SFQGRVTITADESTSTAYMELSSLRSEDTAV | SGSGTEFTLTISSLQSEDFAVYYCQQHGSLPL |
| | YYCARESSSWDYFDYWGQGTLVTVSS (SEQ | TFGQGTKVEIK (SEQ ID NO: 212) |
| | ID NO: 211) | RASQSVRSYLA (SEQ ID NO: 216) |
| | GSGIS (SEQ ID NO: 213) | GASTRAT (SEQ ID NO: 38) |
| | IIYPADSETRYSPSFQG (SEQ ID NO: 214) | QQHGSLPLT (SEQ ID NO: 217) |
| | ESSSWDYFDY (SEQ ID NO: 215) | |
| TMPRSS4 | QVQLVQSGAEVKKPGSSVKVSCKASGGTFSS | DIQMTQSPSSLSASVGDRVTITCRASQSISTY |
| Ab32 | YAISWVRQAPGQGLEWMGRINPSGGSTSYAQ | LNWYQQKPGKAPKLLIYAASSLQRGVPSRFSG |
| | KFQGRVTMTRDTSTSTVYMELSSLRSEDTAV | SGSGTDFTLTISSLQPEDFATYYCQQSYTTPL |
| | YYCARGRYSSSSWGQGTLVTVSS (SEQ ID | TFGGGTKVEIK (SEQ ID NO: 219) |
| | NO: 218) | RASQSISTYLN (SEQ ID NO: 222) |
| | SYAIS (SEQ ID NO: 220) | AASSLQR (SEQ ID NO: 223) |
| | RINPSGGSTSYAQKFQG (SEQ ID NO: 221) | QQSYTTPLT (SEQ ID NO: 224) |
| | GRYSSSS (SEQ ID NO: 81) | |
| TMPRSS4 | QVQLVQSGAEVKKPGASVKVSCKASGYTFSN | DIQMTQSPSSLSASVGDRVTITCRASQYISRW |
| Ab33 | YYMHWVRQAPGQGLEWVGWMNPKSGNTGYAQ | LAWYQQKPGKAPKLLIYGSSTLQSGVPSRFSG |
| | KFQGRVTMTRDTSTSTVYMELSSLRSEDTAV | SGSGTDFTLTISSLQPEDFATYYCQQYYSTPF |
| | YYCARGRTWIQSSLGYWGQGTLVTVSS (SEQ | TFGPGTKLEIK (SEQ ID NO: 226) |
| | ID NO: 225) | RASQYISRWLA (SEQ ID NO: 230) |
| | NYYMH (SEQ ID NO: 227) | GSSTLQS (SEQ ID NO: 231) |
| | WMNPKSGNTGYAQKFQG (SEQ ID NO: 228) | QQYYSTPFT (SEQ ID NO: 232) |
| | GRTWIQSSLGY (SEQ ID NO: 229) | |
| TMPRSS4 | QVQLVQSGAEVKKPGASVKVSCKASGYTFTG | DIQMTQSPSSLSASVGDRVTITCRASQGISSW |
| Ab34 | YYIHWVRQAPGQGLEWMGWMNPHSGNTGYAQ | LAWYQQKPGKAPKLLIYAASTLQTGVPSRFSG |
| | KFQGRVTMTRDTSTSTVYMELSSLRSEDTAV | SGSGTDFTLTISSLQPEDFATYYCQQSKSIPI |
| | YYCAREGGRYSSGRLGYWGQGTLVTVSS | TFGGGTKVEIK (SEQ ID NO: 234) |
| | (SEQ ID NO: 233) | RASQGISSWLA (SEQ ID NO: 238) |
| | GYYIH (SEQ ID NO: 235) | AASTLQT (SEQ ID NO: 239) |
| | WMNPHSGNTGYAQKFQG (SEQ ID NO: 236) | QQSKSIPIT (SEQ ID NO: 240) |
| | EGGRYSSGRLGY (SEQ ID NO: 237) | |
| TMPRSS4 | QVQLVQSGAEVKKPGASVKVSCKASGYTFTG | DIQMTQSPSSLSASVGDRVTITCQASQDISNY |
| Ab35 | YYMHWVRQAPGQGLEWMGKISAHSGETKYAQ | LNWYQQKPGKAPKLLIYKASSLESGVPSRFSG |
| | NVQGRVTMTRDTSTSTVYMELSSLRSEDTAV | SGSGTDFTLTISSLQPEDFATYYCQQTYTIPI |
| | YYCARANYYGDYVNYYYGMDVWGQGTTVTVS | TFGQGTRLEIK (SEQ ID NO: 242) |
| | S (SEQ ID NO: 241) | QASQDISNYLN (SEQ ID NO: 6) |
| | GYYMH (SEQ ID NO: 193) | KASSLES (SEQ ID NO: 7) |
| | KISAHSGETKYAQNVQG (SEQ ID NO: 243) | QQTYTIPIT (SEQ ID NO: 245) |
| | ANYYGDYVNYYYGMDV (SEQ ID NO: 244) | |
| TMPRSS4 | EVQLLESGGGLVQPGGSLRLSCAASGFTESS | DIQMTQSPSSLSASVGDRVTITCRASQSISTW |
| Ab36 | RAMSWVRQAPGKGLEWVSRINYDGSATTYAD | LAWYQQKPGKAPKLLIYRASNLQSGVPSRFSG |
| | SVKGRFTISRDNSKNTLYLQMNSLRAEDTAV | SGSGTDFTLTISSLQPEDFATYYCQQSYSTPL |
| | YYCARGITIFGVFDYWGQGTLVTVSS (SEQ | TFGGGTKVEIK (SEQ ID NO: 247) |
| | ID NO: 246) | RASQSISTWLA (SEQ ID NO: 292) |
| | SRAMS (SEQ ID NO: 248) | RASNLQS (SEQ ID NO: 251) |
| | RINYDGSATTYADSVKG (SEQ ID NO: 249) | QQSYSTPLT (SEQ ID NO: 252) |
| | GITIFGVFDY (SEQ ID NO: 250) | |
| TMPRSS4 | EVQLLESGGGLVQPGGSLRLSCAASGFTFSS | DIQMTQSPSSLSASVGDRVTITCRASQSISRY |
| Ab37 | YAMHWVRQAPGKGLEWVSYISSSGSTVYYAD | LNWYQQKPGKAPKLLIYSASTLQSGVPSRFSG |
| | SVKGRFTISRDNSKNTLYLQMNSLRAEDTAV | SGSGTDFTLTISSLQPEDFATYYCQQAHSFPP |
| | YYCARVSNVTPRSGFGYWGQGTLVTVSS | SFGQGTKLEIK (SEQ ID NO: 254) |
| | (SEQ ID NO: 253) | RASQSISRYLN (SEQ ID NO: 139) |
| | SYAMH (SEQ ID NO: 180) | SASTLQS (SEQ ID NO: 257) |
| | YISSSGSTVYYADSVKG (SEQ ID NO: 255) | QQAHSFPPS (SEQ ID NO: 258) |
| | VSNVTPRSGFGY (SEQ ID NO: 256) | |
| TMPRSS4 | QVQLAQSGAEVKKPGASVKVSCKASGYTFTR | DIQMTQSPSSLSASVGDRVTITCQASQDISRY |
| Ab38 | HYIQWVRQAPGQGLEWMGWINPNSGNTGYAQ | LNWYQQKPGKAPKLLIYGASNLLSGVPSRFSG |
| | KFQGRVTMTRDTSTSTVYMELSSLRSEDTAV | SGSGTDFTLTISSLQPEDFATYYCQQTHTTPY |
| | YYCARGRQWLRGEYFQHWGQGTLVTVSS | TFGQGTRLEIK (SEQ ID NO: 260) |
| | (SEQ ID NO: 259) | QASQDISRYLN (SEQ ID NO: 263) |
| | RHYIQ (SEQ ID NO: 261) | GASNLLS (SEQ ID NO: 264) |
| | WINPNSGNTGYAQKFQG (SEQ ID NO: 50) | QQTHTTPYT (SEQ ID NO: 265) |
| | GRQWLRGEYFQH (SEQ ID NO: 262) | |
| TMPRSS4 | QVQLVQSGAEVKKPGASVKVSCKASGGSFSG | DIQMTQSPSSLSASVGDRVTITCRASQGIRNW |
| Ab39 | YAVSWVRQAPGQGLEWLGVINPSDSWTAFAQ | LAWYQQKPGKAPKLLIYRASTLQSGVPSRFSG |
| | KFQGRVTMTRDTSTSTVYMELSSLRSEDTAV | SGSGTDFTLTISSLQPEDFATYYCQQSYTTPF |
| | YYCAREREDDAFDIWGQGTTVTVSS (SEQ ID | TFGQGTKLEIK (SEQ ID NO: 267) |
| | NO: 266) | RASQGIRNWLA (SEQ ID NO: 271) |
| | GYAVS (SEQ ID NO: 268) | RASTLQS (SEQ ID NO: 272) |
| | VINPSDSWTAFAQKFQG (SEQ ID NO: 269) | QQSYTTPFT (SEQ ID NO: 273) |
| | EREDDAFDI (SEQ ID NO: 270) | |
| TMPRSS4 | QVQLVQSGAEVKKPGASVKVSCKASGGTFSS | DIQMTQSPSSLSASVGDRVTITCRASQSISSY |
| Ab40 | YAISWVRQAPGQGLEWMGIINPRGGSTNYAQ | LNWYQQKPGKAPKLLIYAAYNLQSGVPSRFSG |
| | KFQGRVTMTRDTSTSTVYMELSSLRSEDTAV | SGSGTDFTLTISSLQPEDFATYYCQQSYSIPF |
| | YYCAREGSSWYYDAFDIWGQGTMVTVSS | TFGGGTKVEIK (SEQ ID NO: 275) |
| | (SEQ ID NO: 274) | RASQSISSYLN (SEQ ID NO: 278) |
| | SYAIS (SEQ ID NO: 220) | AAYNLQS (SEQ ID NO: 279) |
| | IINPRGGSTNYAQKFQG (SEQ ID NO: 276) | QQSYSIPFT (SEQ ID NO: 280) |
| | EGSSWYYDAFDI (SEQ ID NO: 277) | |
| TMPRSS4 | QVQLVQSGAEVKKPGASVKVSCKASGGTESS | DIQMTQSPSSLSASVGDRVTITCRASQSISRW |
| Ab41 | YAISWVRQAPGQGLEWMGWMNPNSGDTHYAQ | LAWYQQKPGKAPKLLIYAASTLQTGVPSRESG |
| | KFQGRVTMTRDTSTSTVYMELSSLRSEDTAV | SGSGTDFTLTISSLQPEDFATYYCLQHSSYPF |
| | YYCAREGSSWYYDAFDIWGQGTLVTVSS | TFGQGTKVEIK (SEQ ID NO: 282) |
| | (SEQ ID NO: 281) | RASQSISRWLA (SEQ ID NO: 284) |
| | SYAIS (SEQ ID NO: 220) | AASTLQT (SEQ ID NO: 239) |
| | WMNPNSGDTHYAQKFQG (SEQ ID NO: 283) | LQHSSYPFT (SEQ ID NO: 285) |
| | EGSSWYYDAFDI (SEQ ID NO: 277) | |
| TMPRSS4 | QVQLVQSGAEVKKPGASVKVSCKASGYSFTS | DIQMTQSPSSLSASVGDRVTITCRASQSIRNY |
| Ab42 | HYMHWVRQAPGQGLEWMGWMNPNSGNTGYAQ | LNWYQQKPGKAPKLLIYEASRLQSGVPSRFSG |
| | KFQGRVTMTRDTSTSTVYMELSSLRSEDTAV | SGSGTDFTLTISSLQPEDFATYYCQQSYSAPP |
| | YYCARLGQQLDYWGQGTLVTVSS (SEQ ID | TFGPGTKVDIK (SEQ ID NO: 287) |
| | NO: 286) | RASQSIRNYLN (SEQ ID NO: 290) |
| | SHYMH (SEQ ID NO: 288) | EASRLQS (SEQ ID NO: 291) |
| | WMNPNSGNTGYAQKFQG (SEQ ID NO: 194) | QQSYSAPPT (SEQ ID NO: 293) |
| | LGQQLDY (SEQ ID NO: 289) | |
| TMPRSS4 | QVQLVQSGAEVKKPGASVKVSCKASGYTFIG | DIQMTQSPSSLSASVGDRVTITCQASQDISNY |
| Ab43 | YYMHWVRQAPGQGLEWMGRINPNSGETNYAQ | LNWYQQKPGKAPKLLIYAASSLQSGVPSRFSG |
| | KFQGRVTMTRDTSTSTVYMELSSLRSEDTAV | SGSGTDFTLTISSLQPEDFATYYCQQSYSTPV |
| | YYCARVRVRGVIHPGFDPWGQGTLVTVSS | TFGPGTKVDIK (SEQ ID NO: 295) |
| | (SEQ ID NO: 294) | QASQDISNYLN (SEQ ID NO: 6) |
| | GYYMH (SEQ ID NO: 296) | AASSLQS (SEQ ID NO: 15) |
| | RINPNSGETNYAQKFQG (SEQ ID NO: 297) | QQSYSTPVT (SEQ ID NO: 152) |
| | VRVRGVIHPGFDP (SEQ ID NO: 298) | |
| TMPRSS4 | QVQLVQSGAEVKKPGASVKVSCKASGYTFRN | DIQMTQSPSSLSASVGDRVTITCQASQDISNY |
| Ab44 | YYIHWVRQAPGQGLEWMGRINPNSGGTNYAQ | LNWYQQKPGKAPKLLIYAASSLHSGVPSRFSG |
| | KFQGRVTMTRDTSTSTVYMELSSLRSEDTAV | SGSGTDFTLTISSLQPEDFATYYCQESSSFPY |
| | YYCARARIAVAVSGFGYWGQGTLVTVSS | TFGPGTKVDIK (SEQ ID NO: 300) |
| | (SEQ ID NO: 299) | QASQDISNYLN (SEQ ID NO: 6) |
| | NYYIH (SEQ ID NO: 301) | AASSLHS (SEQ ID NO: 303) |
| | RINPNSGGTNYAQKFQG (SEQ ID NO: 80) | QESSSFPYT (SEQ ID NO: 304) |
| | ARIAVAVSGEGY (SEQ ID NO: 302) | |
| TMPRSS4 | QVQLVQSGAEVKKPGASVKVSCKASGYTFSR | DIQMTQSPSSLSASVGDRVTITCQATQDIRNY |
| Ab45 | WYMHWVRQAPGQGLEWMGRINPNSGGTNYAQ | LNWYQQKPGKAPKLLIYATSSLQSGVPSRFSG |
| | KFQGRVTMTRDTSTSTVYMELSSLRSEDTAV | SGSGTDFTLTISSLQPEDFATYYCQQSYSPPY |
| | YYCARVGGYGWFDPWGQGTLVTVSS (SEQ ID | TFGQGTKLEIK (SEQ ID NO: 306) |
| | NO: 305) | QATQDIRNYLN (SEQ ID NO: 309) |
| | RWYMH (SEQ ID NO: 307) | ATSSLQS (SEQ ID NO: 310) |
| | RINPNSGGTNYAQKFQG (SEQ ID NO: 80) | QQSYSPPYT (SEQ ID NO: 311) |
| | VGGYGWFDP (SEQ ID NO: 308) | |
| TMPRSS4 | QVQLVQSGAEVKKPGASVKVSCKASGYTFSR | DIQMTQSPSSLSASVGDRVTITCRASQSISTW |
| Ab46 | YFMHWVRQAPGQGLEWMGWINPNSGGTNYAQ | LAWYQQKPGKAPKLLIYAASSLQSGVPSRFSG |
| | KFQGRVTMTRDTSTSTVYMELSSLRSEDTAV | SGSGTDFTLTISSLQPEDFATYYCQQSYGFPW |
| | YYCARVRIGWLQSPPLYWGQGTLVTVSS | TFGQGTKVEIK (SEQ ID NO: 313) |
| | (SEQ ID NO: 312) | RASQSISTWLA (SEQ ID NO: 292) |
| | RYFMH (SEQ ID NO: 49) | AASSLQS (SEQ ID NO: 15) |
| | WINPNSGGTNYAQKFQG (SEQ ID NO: 66) | QQSYGFPWT (SEQ ID NO: 314) |
| | VRIGWLQSPPLY (SEQ ID NO: 110) | |
| TMPRSS4 | QVQLVQSGAEVKKPGASVKVSCKASGYTFTG | DIQMTQSPSSLSASVGDRVTITCRASQSISSY |
| Ab47 | YFMHWVRQAPGQGLEWMGWMNPNSGNTGYAQ | LNWYQQKPGKAPKLLIYAASSLQSGVPSRFSG |
| | KFQGRVTMTRDTSTSTVYMELSSLRSEDTAV | SGSGTDFTLTISSLQPEDFATYYCQQSYSTPL |
| | YYCVRGRTWIQSSLGYWGQGTLVTVSS (SEQ | TFGGGTKVEIK (SEQ ID NO: 316) |
| | ID NO: 315) | RASQSISSYLN (SEQ ID NO: 278) |
| | GYFMH (SEQ ID NO: 317) | AASSLQS (SEQ ID NO: 15) |
| | WMNPNSGNTGYAQKFQG (SEQ ID NO: 194) | QQSYSTPLT (SEQ ID NO: 252) |
| | GRTWIQSSLGY (SEQ ID NO: 318) | |
| TMPRSS4 | QVQLVQSGAEVKKPGASVKVSCKASGYTFTG | DIQMTQSPSSLSASVGDRVTITCRASQGISNY |
| Ab48 | YYLHWVRQAPGQGLEWMGWISAYNGNTNYAQ | LAWYQQKPGKAPKLLIYTASTLFPGVPSRFSG |
| | NLQGRVTMTRDTSTSTVYMELSSLRSEDTAV | SGSGTDFTLTISSLQPEDFATYYCQQSYSIPL |
| | YYCARHSYSGSYSTLPYYGMDVWGQGTTVTV | TFGGGTKVEIK (SEQ ID NO: 320) |
| | SS (SEQ ID NO: 319) | RASQGISNYLA (SEQ ID NO: 324) |
| | GYYLH (SEQ ID NO: 321) | TASTLEP (SEQ ID NO: 325) |
| | WISAYNGNTNYAQNLQG (SEQ ID NO: 322) | QQSYSIPLT (SEQ ID NO: 16) |
| | HSYSGSYSTLPYYGMDV (SEQ ID NO: 323) | |
| TMPRSS4 | QVQLVQSGAEVKKPGASVKVSCKASGYTFTG | DIQMTQSPSSLSASVGDRVTITCRASQSISNW |
| Ab49 | YYMHWVRQAPGQGLEWMGRINPNSGGTNYAQ | LAWYQQKPGKAPKLLIYAASTLQNGVPSRFSG |
| | KFQGRVTMTRDTSTSTVYMELSSLRSEDTAV | SGSGTDFTLTISSLQPEDFATYYCQQSYTFPI |
| | YYCARERAGYSSGQFDYWGQGTLVTVSS | TFGQGTKVEIK (SEQ ID NO: 327) |
| | (SEQ ID NO: 326) | RASQSISNWLA (SEQ ID NO: 329) |
| | GYYMH (SEQ ID NO: 193) | AASTLQN (SEQ ID NO: 330) |
| | RINPNSGGTNYAQKFQG (SEQ ID NO: 80) | QQSYTFPIT (SEQ ID NO: 331) |
| | ERAGYSSGQFDY (SEQ ID NO: 328) | |
| TMPRSS4 | QVQLVQSGAEVKKPGASVKVSCKASGYTFTG | DIQMTQSPSSLSASVGDRVTITCRASQGISNY |
| Ab50 | YYMHWVRQAPGQGLEWMGWINPNSGGTHYAQ | LAWYQQKPGKAPKLLIYATSRLQSGVPSRFSG |
| | KFQGRVTMTRDTSTSTVYMELSSLRSEDTAV | SGSGTDFTLTISSLQPEDFATYYCQQSYKTPL |
| | YYCARVRIGWLQSPPLYWGQGTLVTVSS | TFGGGTKVEIK (SEQ ID NO: 333) |
| | (SEQ ID NO: 332) | RASQGISNYLA (SEQ ID NO: 324) |
| | GYYMH (SEQ ID NO: 193) | ATSRLQS (SEQ ID NO: 335) |
| | WINPNSGGTHYAQKFQG (SEQ ID NO: 334) | QQSYKTPLT (SEQ ID NO: 336) |
| | VRIGWLQSPPLY (SEQ ID NO: 110) | |
| TMPRSS4 | QVQLVQSGAEVKKPGASVKVSCKASGYTFTN | DIQMTQSPSSLSASVGDRVTITCRASQSISSY |
| Ab51 | YYMHWVRQAPGQGLEWMGWINPKSGGTSYAQ | LNWYQQKPGKAPKLLIYAASSLQSGVPSRFSG |
| | KFQGRVTMTRDTSTSTVYMELSSLRSEDTAV | SGSGTDFTLTISSLQPEDFATYYCQQSYSTPL |
| | YYCASGKQWLVGGRFDYWGQGTLVTVSS | TFGGGTKVEIK (SEQ ID NO: 316) |
| | (SEQ ID NO: 337) | RASQSISSYLN (SEQ ID NO: 278) |
| | NYYMH (SEQ ID NO: 93) | AASSLQS (SEQ ID NO: 15) |
| | WINPKSGGTSYAQKFQG (SEQ ID NO: 338) | QQSYSTPLT (SEQ ID NO: 252) |
| | GKQWLVGGRFDY (SEQ ID NO: 339) | |
| TMPRSS4 | QVQLVQSGAEVKKPGASVKVSCKASGYTFTR | DIQMTQSPSSLSASVGDRVTITCRASQGISRW |
| Ab52 | YYIHWVRQAPGQGLEWMGWMNPNSGNTGFAQ | LGWYQQKPGKAPKLLIYGASNLQTGVPSRFSG |
| | KLQGRVTMTRDTSTSTVYMELSSLRSEDTAV | SGSGTDFTLTISSLQPEDFATYYCQQSYSSPR |
| | YYCARGPFPRGRLDLWGQGTLVTVSS (SEQ | TFGQGTKVEIK (SEQ ID NO: 341) |
| | ID NO: 340) | RASQGISRWLG (SEQ ID NO: 345) |
| | RYYIH (SEQ ID NO: 342) | GASNLQT (SEQ ID NO: 90) |
| | WMNPNSGNTGFAQKLQG (SEQ ID NO: 343) | QQSYSSPRT (SEQ ID NO: 346) |
| | GPFPRGRLDL (SEQ ID NO: 344) | |
| TMPRSS4 | QVQLVQSGAEVKKPGASVKVSCKASGYTFTR | DIQMTQSPSSLSASVGDRVTITCRASRSINRW |
| Ab53 | YYMHWVRQAPGQGLEWMGIINPTGGSTSYAQ | LAWYQQKPGKAPKLLIYGASTLQSGVPSRFSG |
| | KFQGRVTMTRDTSTSTVYMELSSLRSEDTAV | SGSGTDFTLTISSLQPEDFATYYCQQSYSTPT |
| | YYCARGRTWIQSSLGYWGQGTLVTVSS (SEQ | FGGGTKVEIK (SEQ ID NO: 348) |
| | ID NO: 347) | RASRSINRWLA (SEQ ID NO: 350) |
| | RYYMH (SEQ ID NO: 65) | GASTLQS (SEQ ID NO: 351) |
| | IINPTGGSTSYAQKFQG (SEQ ID NO: 349) | QQSYSTPT (SEQ ID NO: 352) |
| | GRTWIQSSLGY (SEQ ID NO: 229) | |
| TMPRSS4 | QVQLVQSGAEVKKPGASVKVSCKASGYTFTS | DIQMTQSPSSLSASVGDRVTITCRASQGISNY |
| Ab54 | YYMQWVRQAPGQGLEWMGWMNPNSGNTGYAQ | LAWYQQKPGKAPKLLIYAASSLQSGVPSRFSG |
| | KFQGRVTMTRDTSTSTVYMELSSLRSEDTAV | SGSGTDFTLTISSLQPEDFATYYCQQSYSIPF |
| | YYCARVRIGWLQSPPLYWGQGTLVTVSS | TFGPGTKVDIK (SEQ ID NO: 354) |
| | (SEQ ID NO: 353) | RASQGISNYLA (SEQ ID NO: 324) |
| | SYYMQ (SEQ ID NO: 355) | AASSLQS (SEQ ID NO: 15) |
| | WMNPNSGNTGYAQKFQG (SEQ ID NO: 194) | QQSYSIPFT (SEQ ID NO: 280) |
| | VRIGWLQSPPLY (SEQ ID NO: 110) | |
| TMPRSS4 | QVQLVQSGAEVKKPGASVKVSCKASGYTFTT | DIQMTQSPSSLSASVGDRVTITCRASQSISSW |
| Ab55 | YYMHWVRQAPGQGLEWMGIINPSGGSTSYAQ | LAWYQQKPGKAPKLLIYAASSLQSGVPSRFSG |
| | KFQGRVTMTRDTSTSTVYMELSSLRSEDTAV | SGSGTDFTLTISSLQPEDFATYYCQQSYSTPR |
| | YYCARGRSWYRSNVDYWGQGTLVTVSS (SEQ | TFGQGTRLEIK (SEQ ID NO: 357) |
| | ID NO: 356) | RASQSISSWLA (SEQ ID NO: 60) |
| | TYYMH (SEQ ID NO: 207) | AASSLQS (SEQ ID NO: 15) |
| | IINPSGGSTSYAQKFQG (SEQ ID NO: 58) | QQSYSTPRT (SEQ ID NO: 359) |
| | GRSWYRSNVDY (SEQ ID NO: 358) | |
| TMPRSS4 | QVQLVQSGAEVKKPGASVKVSCKASGHTFTR | DIVMTQSPLSLPVTPGEPASISCRSSQSLLHS |
| Ab56 | YYMHWVRQAPGQGLEWMGWINPNSGNTGDAQ | NGYNYLDWYLQKPGQSPQLLIYLGSNRASGVP |
| | KFQGRVTMTRDTSTSTVYMELSSLRSEDTAV | DRFSGSGSGTDFTLKISRVEAEDVGVYYCMQA |
| | YYCARDRGIVVVPAAIGGMDVWGQGTMVTVS | LQTPITFGQGTRLEIK (SEQ ID NO: 361) |
| | S (SEQ ID NO: 360) | RSSQSLLHSNGYNYLD (SEQ ID NO: 29) |
| | RYYMH (SEQ ID NO: 362) | LGSNRAS (SEQ ID NO: 203) |
| | WINPNSGNTGDAQKFQG (SEQ ID NO: 363) | MQALQTPIT (SEQ ID NO: 365) |
| | DRGIVVVPAAIGGMDV (SEQ ID NO: 364) | |
| TMPRSS4 | QVQLVQSGAEVKKPGASVKVSCKASGYTFTG | DIVMTQSPLSLPVTPGEPASISCRSSQSLLHS |
| Ab57 | YFMHWVRQAPGQGLEWMGRINPNSGGTNYAQ | NGYNYLDWYLQKPGQSPQLLIYLGSNRASGVP |
| | KFQGRVTMTRDTSTSTVYMELSSLRSEDTAV | DRFSGSGSGTDFTLKISRVEAEDVGVYYCMQG |
| | YYCARGKGRYFDLWGRGTLVTVSS (SEQ ID | THWPITFGQGTRLEIK (SEQ ID NO: 367) |
| | NO: 366) | RSSQSLLHSNGYNYLD (SEQ ID NO: 29) |
| | GYFMH (SEQ ID NO: 317) | LGSNRAS (SEQ ID NO: 203) |
| | RINPNSGGTNYAQKFQG (SEQ ID NO: 80) | MQGTHWPIT (SEQ ID NO: 369) |
| | GKGRYFDL (SEQ ID NO: 368) | |
| TMPRSS4 | QVQLVQSGAEVKKPGASVKVSCKASGYTFTR | DIVMTQSPLSLPVTPGEPASISCRSSQSLLHS |
| Ab58 | YYLHWVRQAPGQGLEWMGWVSAYNGNTNYAQ | NGYNYLDWYLQKPGQSPQLLIYLGSNRASGVP |
| | KFQGRVTMTRDTSTSTVYMELSSLRSEDTAV | DRFSGSGSGTDFTLKISRVEAEDVGVYYCMQA |
| | YYCARGYCSGGSCYWFDPWGQGTLVTVSS | LQTPLTFGQGTKVEIK (SEQ ID NO: 371) |
| | (SEQ ID NO: 370) | RSSQSLLHSNGYNYLD (SEQ ID NO: 29) |
| | RYYLH (SEQ ID NO: 372) | LGSNRAS (SEQ ID NO: 203) |
| | WVSAYNGNTNYAQKFQG (SEQ ID NO: 373) | MQALQTPLT (SEQ ID NO: 375) |
| | GYCSGGSCYWFDP (SEQ ID NO: 374) | |
| TMPRSS4 | QVQLVQSGAEVKKPGASVKVSCKASGGTFSS | EIVMTQSPATLSVSPGERATLSCRASQSVSSN |
| Ab59 | YTLSWVRQAPGQGLEWMGWIHPKSGVTKNAQ | YLAWYQQKPGQAPRLLIYGASTRATGIPARFS |
| | KFQGRVTMTRDTSTSTVYMELSSLRSEDTAV | GSGSGTEFTLTISSLQSEDFAVYYCQQYGTLP |
| | YYCARGWVYGRMDAWGQGTTVTVSS (SEQ ID | YTFGQGTKVEIK (SEQ ID NO: 377) |
| | NO: 376) | RASQSVSSNYLA (SEQ ID NO: 175) |
| | SYTLS (SEQ ID NO: 378) | GASTRAT (SEQ ID NO: 38) |
| | WIHPKSGVTKNAQKFQG (SEQ ID NO: 379) | QQYGTLPYT (SEQ ID NO: 381) |
| | GWVYGRMDA (SEQ ID NO: 380) | |
| TMPRSS4 | QVQLVQSGAEVKKPGASVKVSCKASGYSFTT | EIVMTQSPATLSVSPGERATLSCRASQSVSSN |
| Ab60 | YYIHWVRQAPGQGLEWMGIINPSGGSTSYAQ | TLAWYQQKPGQAPRLLIYGASTRATGIPARFS |
| | KFQGRVTMTRDTSTSTVYMELSSLRSEDTAV | GSGSGTEFTLTISSLQSEDFAVYYCQQYGSSP |
| | YYCARGGYYGSGYNSVGYWGPGTLVTVSS | LTFGPGTKVDIK (SEQ ID NO: 383) |
| | (SEQ ID NO: 382) | RASQSVSSNTLA (SEQ ID NO: 386) |
| | TYYIH (SEQ ID NO: 384) | GASTRAT (SEQ ID NO: 38) |
| | IINPSGGSTSYAQKFQG (SEQ ID NO: 58) | QQYGSSPLT (SEQ ID NO: 387) |
| | GGYYGSGYNSVGY (SEQ ID NO: 385) | |
| TMPRSS4 | QVQLVQSGAEVKKPGASVKVSCKASGYTFTN | EIVMTQSPATLSVSPGERATLSCRASQSVSSY |
| Ab61 | YYMHWVRQAPGQGLEWMGWMNPNSGNTGYAQ | LAWYQQKPGQAPRLLIYGASTRATGIPARFSG |
| | NLQGRVTMTRDTSTSTVYMELSSLRSEDTAV | SGSGTEFTLTISSLQSEDFAVYYCQQYDISVT |
| | YYCARGRTWFRSGMDVWGQGTTVTVSS (SEQ | FGPGTKVDIK (SEQ ID NO: 389) |
| | ID NO: 388) | RASQSVSSYLA (SEQ ID NO: 75) |
| | NYYMH (SEQ ID NO: 93) | GASTRAT (SEQ ID NO: 38) |
| | WMNPNSGNTGYAQNLQG (SEQ ID NO: 390) | QQYDISVT (SEQ ID NO: 391) |
| | GRTWFRSGMDV (SEQ ID NO: 163) | |
| TMPRSS4 | QVQLVQSGAEVKKPGASVKVSCKASGYTFTD | DIVMTQSPDSLAVSLGERATINCKSSQSVLYS |
| Ab62 | YYIHWVRQAPGQGLEWMGWISTYNGNTNYAQ | SNNKNYLAWYQQKPGQPPKLLIYWASTRESGV |
| | KLQGRVTMTRDTSTSTVYMELSSLRSEDTAV | PDRFSGSGSGTDFTLTISSLQAEDVAVYYCQQ |
| | YYCARGMVRGMDVWGQGTMVTVSS (SEQ ID | YYTTPWTFGQGTRLEIK (SEQ ID NO: 393) |
| | NO: 392) | KSSQSVLYSSNNKNYLA (SEQ ID NO: 44) |
| | DYYIH (SEQ ID NO: 394) | WASTRES (SEQ ID NO: 45) |
| | WISTYNGNTNYAQKLQG (SEQ ID NO: 395) | QQYYTTPWT (SEQ ID NO: 397) |
| | GMVRGMDV (SEQ ID NO: 396) | |
| TMPRSS4 | QVQLVQSGAEVKKPGASVKVSCKASGYTFTG | DIVMTQSPDSLAVSLGERATINCKSSQSVLYS |
| Ab63 | YRMHWVRQAPGQGLEWMGVINPNTGTARFAQ | SNNKNYLAWYQQKPGQPPKLLIYWASTRESGV |
| | KFQGRVTMTRDTSTSTVYMELSSLRSEDTAV | PDRFSGSGSGTDFTLTISSLQAEDVAVYYCQQ |
| | YYCASVGVYWYFDLWGRGTLVTVSS (SEQ ID | YYSAPLTFGGGTKVEIK (SEQ ID NO: 399) |
| | NO: 398) | KSSQSVLYSSNNKNYLA (SEQ ID NO: 44) |
| | GYRMH (SEQ ID NO: 400) | WASTRES (SEQ ID NO: 45) |
| | VINPNTGTARFAQKFQG (SEQ ID NO: 401) | QQYYSAPLT (SEQ ID NO: 403) |
| | VGVYWYFDL (SEQ ID NO: 402) | |
| TMPRSS4 | QVQLVQSGAEVKKPGASVKVSCKASGYTFTG | DIVMTQSPDSLAVSLGERATINCKSSQSVLYS |
| Ab64 | YYMHWVRQAPGQGLEWMGMINPSGGGTTYAQ | SNNKNYLAWYQQKPGQPPKLLIYWASTRESGV |
| | KFQGRVTMTRDTSTSTVYMELSSLRSEDTAV | PDRFSGSGSGTDFTLTISSLQAEDVAVYYCQQ |
| | YYCARDRRSMITFRTDYWGQGTLVTVSS | YYSTPYTFGQGTKLEIK (SEQ ID NO: 405) |
| | (SEQ ID NO: 404) | KSSQSVLYSSNNKNYLA (SEQ ID NO: 44) |
| | GYYMH (SEQ ID NO: 193) | WASTRES (SEQ ID NO: 45) |
| | MINPSGGGTTYAQKFQG (SEQ ID NO: 406) | QQYYSTPYT (SEQ ID NO: 408) |
| | DRRSMITFRTDY (SEQ ID NO: 407) | |
| TMPRSS4 | QVQLVQSGAEVKKPGASVKVSCKASGYTFTG | DIVMTQSPDSLAVSLGERATINCKSSQSVLYS |
| Ab65 | YYMHWVRQAPGQGLEWMGWMNPNSGNTGYAQ | SNNKNYLAWYQQKPGQPPKLLIYWASTRQSGV |
| | KFQGRVTMTRDTSTSTVYMELSSLRSEDTAV | PDRFSGSGSGTDFTLTISSLQAEDVAVYYCQQ |
| | YYCAGRKWLGLDFYNWFDPWGQGTLVTVSS | YYSTPWTFGQGTKVEIK (SEQ ID NO: 410) |
| | (SEQ ID NO: 409) | KSSQSVLYSSNNKNYLA (SEQ ID NO: 44) |
| | GYYMH (SEQ ID NO: 193) | WASTRQS (SEQ ID NO: 412) |
| | WMNPNSGNTGYAQKFQG (SEQ ID NO: 194) | QQYYSTPWT (SEQ ID NO: 413) |
| | RKWLGLDFYNWFDP (SEQ ID NO: 411) | |
| TMPRSS4 | QVQLVQSGAEVKKPGSSVKVSCKASGGTFSS | EIVMTQSPATLSVSPGERATLSCRASQSVNSR |
| Ab66 | YAISWVRQAPGQGLEWVGGIMPIFGTANYAQ | FLAWYQQKPGQAPRLLIYGASTRATGIPARFS |
| | KFQGRVTITADESPSTAYMELSSLRSEDTAV | GSGSGTEFTLTISSLQSEDFAVYYCMQGTHWP |
| | YYCATGRRELLNWGQGTLVTVSS (SEQ ID | YTFGQGTKVEIK (SEQ ID NO: 415) |
| | NO: 414) | RASQSVNSRFLA (SEQ ID NO: 418) |
| | SYAIS (SEQ ID NO: 220) | GASTRAT (SEQ ID NO: 38) |
| | GIMPIFGTANYAQKFQG (SEQ ID NO: 416) | MQGTHWPYT (SEQ ID NO: 419) |
| | GRRELLN (SEQ ID NO: 417) | |
| TMPRSS4 | QVQLVQSGAEVKKPGSSVKVSCKASGYTFTS | DIVMTQSPDSLAVSLGERATINCKSSQSVLYS |
| Ab67 | YDINWVRQAPGQGLEWMGGIIPIFGTANYAQ | SNNKNYLAWYQQKPGQPPKLLIYWASTRESGV |
| | KFQGRVTITADESTSTAYMELSSLRSEDTAV | PDRFSGSGSGTDFTLTISSLQAEDVAVYYCQQ |
| | YYCATTPGDAFDIWGQGTMVTVSS (SEQ ID | YSDTPLTFGQGTKVEIK (SEQ ID NO: 421) |
| | NO: 420) | KSSQSVLYSSNNKNYLA (SEQ ID NO: 44) |
| | SYDIN (SEQ ID NO: 19) | WASTRES (SEQ ID NO: 45) |
| | GIIPIFGTANYAQKFQG (SEQ ID NO: 4) | QQYSDTPLT (SEQ ID NO: 423) |
| | TPGDAFDI (SEQ ID NO: 422) | |
| TMPRSS4 | EVQLLESGGGLVKPGGSLRLSCAASGFTFSS | DIQMTQSPSSLSASVGDRVTITCRASQSISRY |
| Ab68 | SWMHWVRQAPGKGLEWVSAIGTAGDTYYPGS | LNWYQQKPGKAPKLLIYAASSLQSGVPSRFSG |
| | VKGRFTISRDDSKNTLYLQMNSLKTEDTAVY | SGSGTDFTLTISSLQPEDFATYYCQQSYSNPP |
| | YCARVRLGHFDLWGRGTLVTVSS (SEQ ID | TFGQGTKLEIK (SEQ ID NO: 425) |
| | NO: 424) | RASQSISRYLN (SEQ ID NO: 139) |
| | SSWMH (SEQ ID NO: 426) | AASSLQS (SEQ ID NO: 15) |
| | AIGTAGDTYYPGSVKG (SEQ ID NO: 427) | QQSYSNPPT (SEQ ID NO: 429) |
| | VRLGHFDL (SEQ ID NO: 428) | |
| TMPRSS4 | EVQLLESGGGLVQHGGSLRLSCAASGFAFSS | DIQMTQSPSSLSASVGDRVTITCRASQGISSW |
| Ab69 | YVLHWVRQAPGKGLEWVSSISSSSSYIYYAD | LAWYQQKPGKAPKLLIYQASNKDTGVPSRFSG |
| | SVKGRFTISRDNSKNTLYLQMNSLRAEDTAV | SGSGTDFTLTISSLQPEDFATYYCQQSYRIPW |
| | YYCARGDRYPGLPNYWGQGTLVTVSS (SEQ | TFGQGTKVEIK (SEQ ID NO: 431) |
| | ID NO: 430) | RASQGISSWLA (SEQ ID NO: 238) |
| | SYVLH (SEQ ID NO: 432) | QASNKDT (SEQ ID NO: 435) |
| | SISSSSSYIYYADSVKG (SEQ ID NO: 433) | QQSYRIPWT (SEQ ID NO: 436) |
| | GDRYPGLPNY (SEQ ID NO: 434) | |
| TMPRSS4 | EVQLLESGGGLVQPGGSLRLSCAASGFAFSG | DIQMTQSPSSLSASVGDRVTITCRASQSISGW |
| Ab70 | TWMQWVRQAPGKGLEWVSDISGSSRDTNYAD | LAWYQQKPGKAPKLLIYAASTLRDGVPSRFSG |
| | SVKGRFTISRDNSKNTLYLQMNSLRAEDTAV | SGSGTDFTLTISSLQPEDFATYYCQQANSFPL |
| | YYCAKDHWDSYGYLDYWGQGTLVTVSS (SEQ | TFGQGTKVEIK (SEQ ID NO: 438) |
| | ID NO: 437) | RASQSISGWLA (SEQ ID NO: 442) |
| | GTWMQ (SEQ ID NO: 439) | AASTLRD (SEQ ID NO: 443) |
| | DISGSSRDTNYADSVKG (SEQ ID NO: 440) | QQANSFPLT (SEQ ID NO: 444) |
| | DHWDSYGYLDY (SEQ ID NO: 441) | |
| TMPRSS4 | EVQLLESGGGLVQPGGSLRLSCAASGFMFDY | DIQMTQSPSSLSASVGDRVTITCRASQGISNN |
| Ab71 | YAMHWVRQAPGKGLEWVSLISYDGRNKYYAD | LNWYQQKPGKAPKLLIYAASSLQSGVPSRFSG |
| | SVKGRFTISRDNSKNTLYLQMNSLRAEDTAV | SGSGTDFTLTISSLQPEDFATYYCQQANNFPI |
| | YYCARPGSYSRFQHWGQGTLVTVSS (SEQ ID | TFGQGTKVEIK (SEQ ID NO: 446) |
| | NO: 445) | RASQGISNNLN (SEQ ID NO: 450) |
| | YYAMH (SEQ ID NO: 447) | AASSLQS (SEQ ID NO: 15) |
| | LISYDGRNKYYADSVKG (SEQ ID NO: 448) | QQANNFPIT (SEQ ID NO: 451) |
| | PGSYSRFQH (SEQ ID NO: 449) | |
| TMPRSS4 | EVQLLESGGGLVQPGGSLRLSCAASGFTFGA | DIQMTQSPSSLSASVGDRVTITCRASQNISRW |
| Ab72 | YVMHWVRQAPGKGLEWVSSISGGSTYYADSV | LAWYQQKPGKAPKLLIYAASSLQSGVPSRFSG |
| | KGRFTISRDNSKNTLYLQMNSLRAEDTAVYY | SGSGTDFTLTISSLQPEDFATYYCQQAISFPL |
| | CARHPVRGVIGAGWFDPWGQGTLVTVSS | TFGGGTKVEIK (SEQ ID NO: 453) |
| | (SEQ ID NO: 452) | RASQNISRWLA (SEQ ID NO: 457) |
| | AYVMH (SEQ ID NO: 454) | AASSLQS (SEQ ID NO: 15) |
| | SISGGSTYYADSVKG (SEQ ID NO: 455) | QQAISFPLT (SEQ ID NO: 112) |
| | HPVRGVIGAGWFDP (SEQ ID NO: 456) | |
| TMPRSS4 | EVQLLESGGGLVQPGGSLRLSCAASGFTFSS | DIQMTQSPSSLSASVGDRVTITCRASQGISNS |
| Ab73 | YAMHWVRQAPGKGLEWLAVISEDGSIRHYAD | LAWYQQKPGKAPKLLIYSAVNLQSGVPSRFSG |
| | SVKGRFTISRDNSKNTLYLQMNSLRAEDTAV | SGSGTDFTLTISSLQPEDFATYYCQQANSFPL |
| | YYCAKPKASSGPRLIDYWGQGTLVTVSS | TFGGGTKVEIK (SEQ ID NO: 459) |
| | (SEQ ID NO: 458) | RASQGISNSLA (SEQ ID NO: 462) |
| | SYAMH (SEQ ID NO: 180) | SAVNLQS (SEQ ID NO: 463) |
| | VISFDGSIRHYADSVKG (SEQ ID NO: 460) | QQANSFPLT (SEQ ID NO: 464) |
| | PKASSGPRLIDY (SEQ ID NO: 461) | |
| TMPRSS4 | EVQLLESGGGLVQPGGSLRLSCAASGFTFSS | DIQMTQSPSSLSASVGDRVTITCRASQSVSSW |
| Ab74 | YAMHWVRQAPGKGLEWVSSISSSSTYIHYAD | LAWYQQKPGKAPKLLIYDASSLQSGVPSRFSG |
| | SVKGRFTISRDNSKNTLYLQMNSLRAEDTAV | SGSGTDFTLTISSLQPEDFATYYCQQAKSFPP |
| | YYCARVGRYYGSGSSLVDYWGQGTLVTVSS | TFGQGTKVEIK (SEQ ID NO: 466) |
| | (SEQ ID NO: 465) | RASQSVSSWLA (SEQ ID NO: 469) |
| | SYAMH (SEQ ID NO: 180) | DASSLQS (SEQ ID NO: 470) |
| | SISSSSTYIHYADSVKG (SEQ ID NO: 467) | QQAKSFPPT (SEQ ID NO: 471) |
| | VGRYYGSGSSLVDY (SEQ ID NO: 468) | |
| TMPRSS4 | EVQLLESGGGLVQPGGSLRLSCAASGFTESS | DIQMTQSPSSLSASVGDRVTITCRASQGIRND |
| Ab75 | YAMSWVRQAPGKGLEWVSSISSASSYKYYAD | LNWYQQKPGKAPKLLIYAATRLQSGVPSRFSG |
| | SVKGRFTISRDNSKNTLYLQMNSLRAEDTAV | SGSGTDFTLTISSLQPEDFATYYCQQAHSFPY |
| | YYCARDIYSSGWRGYYYYGMDVWGQGTTVTV | SFGQGTRLEIK (SEQ ID NO: 473) |
| | SS (SEQ ID NO: 472) | RASQGIRNDLN (SEQ ID NO: 477) |
| | SYAMS (SEQ ID NO: 474) | AATRLQS (SEQ ID NO: 478) |
| | SISSASSYKYYADSVKG (SEQ ID NO: 475) | QQAHSFPYS (SEQ ID NO: 479) |
| | DIYSSGWRGYYYYGMDV (SEQ ID NO: 476) | |
| TMPRSS4 | EVQLLESGGGLVQPGGSLRLSCAASGFTFSS | DIVMTQSPDSLAVSLGERATINCKSSQSVLYS |
| Ab76 | YAMSWVRQAPGKGLEWVSAISGSGGNAYYAD | SNNKNYLAWYQQKPGQPPKLLIYWASTRASGV |
| | SVKGRFTISRDNAKNSLYLQMNSLRAEDTAV | PDRFSGSGSGTDFTLTISSLQAEDVAVYYCQQ |
| | YYCAKNSWGSYRPRAFDIWGQGTMVTVSS | YLSLPYTFGQGTKVEIK (SEQ ID NO: 481) |
| | (SEQ ID NO: 480) | KSSQSVLYSSNNKNYLA (SEQ ID NO: 44) |
| | SYAMS (SEQ ID NO: 474) | WASTRAS (SEQ ID NO: 484) |
| | AISGSGGNAYYADSVKG (SEQ ID NO: 482) | QQYLSLPYT (SEQ ID NO: 485) |
| | NSWGSYRPRAFDI (SEQ ID NO: 483) | |
| TMPRSS4 | QVQLVQSGAEVKKPGASVKVSCKASGNIFTA | DIVMTQSPDSLAVSLGERATINCKSSQSVLYS |
| Ab77 | QYMHWVRQAPGQGLEWMGWMNPNTVYTGSAQ | SNNKNYLAWYQQKPGQPPKLLIYWASTRESGV |
| | KFQGRVTMTRDTSTSTVYMELSSLRSEDTAV | PDRFSGSGSGTDFTLTISSLQAEDVAVYYCQQ |
| | YYCARDWVGDGYNSFDYWGQGTLVTVSS | YYTTPFTFGPGTKVDIK (SEQ ID NO: 487) |
| | (SEQ ID NO: 486) | KSSQSVLYSSNNKNYLA (SEQ ID NO: 44) |
| | AQYMH (SEQ ID NO: 488) | WASTRES (SEQ ID NO: 45) |
| | WMNPNTVYTGSAQKFQG (SEQ ID NO: 489) | QQYYTTPFT (SEQ ID NO: 491) |
| | DWVGDGYNSFDY (SEQ ID NO: 490) | |
| TMPRSS4 | EVQLLESGGGLVQPGGSLRLSCAASGFTFSS | DIQMTQSPSSLSASVGDRVTITCRASQDIKNF |
| Ab78 | YGMNWVRQAPGKGLEWVSAISGSGGRTYYAD | LAWYQQKPGKAPKLLIYAASSLQSGVPSRFSG |
| | SVKGRFTISRDNAKNSLYLQMNSLRAEDTAV | SGSGTDFTLTISSLQPEDFATYYCQQSYSTPW |
| | YYCAKGTYYSSPKYSFDYWGQGTLVTVSS | TFGQGTKLEIK (SEQ ID NO: 493) |
| | (SEQ ID NO: 492) | RASQDIKNFLA (SEQ ID NO: 497) |
| | SYGMN (SEQ ID NO: 494) | AASSLQS (SEQ ID NO: 15) |
| | AISGSGGRTYYADSVKG (SEQ ID NO: 495) | QQSYSTPWT (SEQ ID NO: 498) |
| | GTYYSSPKYSEDY (SEQ ID NO: 496) | |
| TMPRSS4 | EVQLLESGGGLVQPGGSLRLSCAASGLTFSS | DIQMTQSPSSLSASVGDRVTITCRASQGISNY |
| Ab79 | YQMSSVSQAPGKGLEWVSYISSAANTVYYAD | LAWYQQKPGKAPKLLIYAASSLQSGVPSRFSG |
| | SVKGRFTISRDNSKNTLYLQMNSLRAEDTAV | SGSGTDFTLTISSLQPEDFATYYCQQSYSTPL |
| | YYCAREDESRSPYCSGGSCYRAEYFQHWGQG | TFGPGTKVDIK (SEQ ID NO: 500) |
| | TLVTVSS (SEQ ID NO: 499) | RASQGISNYLA (SEQ ID NO: 324) |
| | SYQMS (SEQ ID NO: 501) | AASSLQS (SEQ ID NO: 15) |
| | YISSAANTVYYADSVKG (SEQ ID NO: 502) | QQSYSTPLT (SEQ ID NO: 252) |
| | EDESRSPYCSGGSCYRAEYFQH (SEQ ID | |
| | NO: 503) | |

In some embodiments, the antibody or antigen-binding fragment that binds to human TMPRSS4 comprises a heavy chain variable domain (VH) comprising a heavy chain complementarity determining region 1 (HCDR1), a heavy chain complementarity determining region 2 (HCDR2), and a heavy chain complementarity determining region 3 (HCDR3), and a light chain variable domain (VL) comprising a light chain complementarity determining region 1 (LCDR1), a light chain complementarity determining region 2 (LCDR2), and a light chain complementarity determining region (LCDR3), wherein the HCDR1, HCDR2, HCDR3, LCDR1, LCDR2, and LCDR3 are each from a clone listed in Table 3. In some embodiments, the combination of six CDRs (a CDR-H1, a CDR-H2, a CDR-H3, a CDR-L1, a CDR-L2 and a CDR-L3) is according to Kabat, Chothia, AbM, IMGT, or Contact numbering.

In some embodiments, the antibody or antigen-binding fragment that binds to human TMPRSS4 comprises a VH and a VL each comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence of a VH and a VL of a clone listed in Table 3, optionally wherein the VH CDRs and the VL CDRs are identical to those of the respective sequences in Table 3.

In some embodiments, the antibody or antigen-binding fragment that binds to human TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to an amino acid sequence as set forth in SEQ ID NOs: 1, 9, 17, 24, 32, 40, 47, 55, 63, 71, 77, 84, 91, 99, 107, 113, 120, 128, 134, 140, 147, 153, 159, 166, 171, 178, 185, 191, 199, 205, 211, 218, 225, 233, 241, 246, 253, 259, 266, 274, 281, 286, 294, 299, 305, 312, 315, 319, 326, 332, 337, 340, 347, 353, 356, 360, 366, 370, 376, 382, 388, 392, 398, 404, 409, 414, 420, 424, 430, 437, 554, 452, 458, 465, 472, 480, 486, 492, or 499, optionally wherein the VH CDRs and the VL CDRs are identical to those in the respective sequence. In some embodiments, the antibody or antigen-binding fragment that binds to TMPRSS4 comprises a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to an amino acid sequence as set forth in SEQ ID NOs: 2, 10, 18, 25, 33, 41, 48, 56, 64, 72, 78, 85, 92, 100, 108, 114, 121, 129, 135, 141, 148, 154, 160, 167, 172, 179, 186, 192, 200, 206, 212, 219, 226, 234, 242, 247, 254, 260, 267, 275, 282, 287, 295, 300, 306, 313, 316, 320, 327, 333, 316, 341, 348, 254, 357, 361, 367, 371, 377, 383, 389, 393, 399, 405, 410, 415, 421, 425, 431, 438, 446, 435, 459, 466, 473, 481, 487, 493, or 500, optionally wherein the VH CDRs and the VL CDRs are identical to those in the respective sequence. In some embodiments, the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to an amino acid sequence as set forth in SEQ ID NOs: 1, 9, 17, 24, 32, 40, 47, 55, 63, 71, 77, 84, 91, 99, 107, 113, 120, 128, 134, 140, 147, 153, 159, 166, 171, 178, 185, 191, 199, 205, 211, 218, 225, 233, 241, 246, 253, 259, 266, 274, 281, 286, 294, 299, 305, 312, 315, 319, 326, 332, 337, 340, 347, 353, 356, 360, 366, 370, 376, 382, 388, 392, 398, 404, 409, 414, 420, 424, 430, 437, 554, 452, 458, 465, 472, 480, 486, 492, or 499, optionally wherein the VH CDRs and the VL CDRs are identical to those in the respective sequence, and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to an amino acid sequence as set forth in SEQ ID NO: 2, 10, 18, 25, 33, 41, 48, 56, 64, 72, 78, 85, 92, 100, 108, 114, 121, 129, 135, 141, 148, 154, 160, 167, 172, 179, 186, 192, 200, 206, 212, 219, 226, 234, 242, 247, 254, 260, 267, 275, 282, 287, 295, 300, 306, 313, 316, 320, 327, 333, 316, 341, 348, 254, 357, 361, 367, 371, 377, 383, 389, 393, 399, 405, 410, 415, 421, 425, 431, 438, 446, 435, 459, 466, 473, 481, 487, 493, or 500, optionally wherein the VH CDRs and the VL CDRs are identical to those in the respective sequence.

In some embodiments, the antibody or antigen-binding fragment that binds to human TMPRSS4 is or is derived from TMPRSS4 Ab1. In some embodiments, the antibody or antigen-binding fragment that binds to TMPRSS4 comprises a VH comprising an HCDR1 comprising the amino acid sequence set forth in SEQ ID NO: 3, an HCDR2 comprising the amino acid sequence set forth in SEQ ID NO: 4, and an HCDR3 comprising the amino acid sequence set forth in SEQ ID NO: 5, and a VL comprising an LCDR1 comprising the amino acid sequence set forth in SEQ ID NO: 6, an LCDR2 comprising the amino acid sequence set forth in SEQ ID NO: 7, and an LCDR3 comprising the amino acid sequence set forth in SEQ ID NO: 8.

In some embodiments, the antibody or antigen-binding fragment that binds to human TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 1 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 2. In some embodiments, to TMPRSS4 comprises a VH comprising the amino acid sequence set forth in SEQ ID NO: 1 and a VL comprising the amino acid sequence set forth in SEQ ID NO: 2.

In some embodiments, the antibody or antigen-binding fragment that binds to human TMPRSS4 is or is derived from TMPRSS4 Ab2. In some embodiments, the antibody or antigen-binding fragment that binds to TMPRSS4 comprises a VH comprising an HCDR1 comprising the amino acid sequence set forth in SEQ ID NO: 11, an HCDR2 comprising the amino acid sequence set forth in SEQ ID NO: 12, and an HCDR3 comprising the amino acid sequence set forth in SEQ ID NO: 13, and a VL comprising an LCDR1 comprising the amino acid sequence set forth in SEQ ID NO: 14, an LCDR2 comprising the amino acid sequence set forth in SEQ ID NO: 15, and an LCDR3 comprising the amino acid sequence set forth in SEQ ID NO: 16 and/or, the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 9 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 10. In some embodiments, to TMPRSS4 comprises a VH comprising the amino acid sequence set forth in SEQ ID NO: 9 and a VL comprising the amino acid sequence set forth in SEQ ID NO: 10.

In some embodiments, the antibody or antigen-binding fragment that binds to human TMPRSS4 is or is derived from TMPRSS4 Ab3. In some embodiments, the antibody or antigen-binding fragment that binds to TMPRSS4 comprises a VH comprising an HCDR1 comprising the amino acid sequence set forth in SEQ ID NO: 19, an HCDR2 comprising the amino acid sequence set forth in SEQ ID NO: 20, and an HCDR3 comprising the amino acid sequence set forth in SEQ ID NO: 21, and a VL comprising an LCDR1 comprising the amino acid sequence set forth in SEQ ID NO: 22, an LCDR2 comprising the amino acid sequence set forth in SEQ ID NO: 15, and an LCDR3 comprising the amino acid sequence set forth in SEQ ID NO: 23 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 17 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 18. In some embodiments, to TMPRSS4 comprises a VH comprising the amino acid sequence set forth in SEQ ID NO: 17 and a VL comprising the amino acid sequence set forth in SEQ ID NO: 18.

In some embodiments, the antibody or antigen-binding fragment that binds to human TMPRSS4 is or is derived from TMPRSS4 Ab4. In some embodiments, the antibody or antigen-binding fragment that binds to TMPRSS4 comprises a VH comprising an HCDR1 comprising the amino acid sequence set forth in SEQ ID NO: 26, an HCDR2 comprising the amino acid sequence set forth in SEQ ID NO: 27, and an HCDR3 comprising the amino acid sequence set forth in SEQ ID NO: 28, and a VL comprising an LCDR1 comprising the amino acid sequence set forth in SEQ ID NO: 29, an LCDR2 comprising the amino acid sequence set forth in SEQ ID NO: 30, and an LCDR3 comprising the amino acid sequence set forth in SEQ ID NO: 31 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 24 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 25. In some embodiments, to TMPRSS4 comprises a VH comprising the amino acid sequence set forth in SEQ ID NO: 24 and a VL comprising the amino acid sequence set forth in SEQ ID NO: 25.

In some embodiments, the antibody or antigen-binding fragment that binds to human TMPRSS4 is or is derived from TMPRSS4 Ab5. In some embodiments, the antibody or antigen-binding fragment that binds to TMPRSS4 comprises a VH comprising an HCDR1 comprising the amino acid sequence set forth in SEQ ID NO: 34, an HCDR2 comprising the amino acid sequence set forth in SEQ ID NO: 35, and an HCDR3 comprising the amino acid sequence set forth in SEQ ID NO: 36, and a VL comprising an LCDR1 comprising the amino acid sequence set forth in SEQ ID NO: 37, an LCDR2 comprising the amino acid sequence set forth in SEQ ID NO: 38, and an LCDR3 comprising the amino acid sequence set forth in SEQ ID NO: 39 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 32 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 33. In some embodiments, to TMPRSS4 comprises a VH comprising the amino acid sequence set forth in SEQ ID NO: 32 and a VL comprising the amino acid sequence set forth in SEQ ID NO: 33.

In some embodiments, the antibody or antigen-binding fragment that binds to human TMPRSS4 is or is derived from TMPRSS4 Ab6. In some embodiments, the antibody or antigen-binding fragment that binds to TMPRSS4 comprises a VH comprising an HCDR1 comprising the amino acid sequence set forth in SEQ ID NO: 34, an HCDR2 comprising the amino acid sequence set forth in SEQ ID NO: 42, and an HCDR3 comprising the amino acid sequence set forth in SEQ ID NO: 43, and a VL comprising an LCDR1 comprising the amino acid sequence set forth in SEQ ID NO: 44, an LCDR2 comprising the amino acid sequence set forth in SEQ ID NO: 45, and an LCDR3 comprising the amino acid sequence set forth in SEQ ID NO: 46 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 40 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 41. In some embodiments, to TMPRSS4 comprises a VH comprising the amino acid sequence set forth in SEQ ID NO: 40 and a VL comprising the amino acid sequence set forth in SEQ ID NO: 41.

In some embodiments, the antibody or antigen-binding fragment that binds to human TMPRSS4 is or is derived from TMPRSS4 Ab7. In some embodiments, the antibody or antigen-binding fragment that binds to TMPRSS4 comprises a VH comprising an HCDR1 comprising the amino acid sequence set forth in SEQ ID NO: 49, an HCDR2 comprising the amino acid sequence set forth in SEQ ID NO: 50, and an HCDR3 comprising the amino acid sequence set forth in SEQ ID NO: 51, and a VL comprising an LCDR1 comprising the amino acid sequence set forth in SEQ ID NO: 52, an LCDR2 comprising the amino acid sequence set forth in SEQ ID NO: 53, and an LCDR3 comprising the amino acid sequence set forth in SEQ ID NO: 54 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 47 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 48. In some embodiments, to TMPRSS4 comprises a VH comprising the amino acid sequence set forth in SEQ ID NO: 47 and a VL comprising the amino acid sequence set forth in SEQ ID NO: 48.

In some embodiments, the antibody or antigen-binding fragment that binds to human TMPRSS4 is or is derived from TMPRSS4 Ab8. In some embodiments, the antibody or antigen-binding fragment that binds to TMPRSS4 comprises a VH comprising an HCDR1 comprising the amino acid sequence set forth in SEQ ID NO: 57, an HCDR2 comprising the amino acid sequence set forth in SEQ ID NO: 58, and an HCDR3 comprising the amino acid sequence set forth in SEQ ID NO: 59, and a VL comprising an LCDR1 comprising the amino acid sequence set forth in SEQ ID NO: 60, an LCDR2 comprising the amino acid sequence set forth in SEQ ID NO: 61, and an LCDR3 comprising the amino acid sequence set forth in SEQ ID NO: 62 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 55 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 56. In some embodiments, to TMPRSS4 comprises a VH comprising the amino acid sequence set forth in SEQ ID NO: 55 and a VL comprising the amino acid sequence set forth in SEQ ID NO: 56.

In some embodiments, the antibody or antigen-binding fragment that binds to human TMPRSS4 is or is derived from TMPRSS4 Ab9. In some embodiments, the antibody or antigen-binding fragment that binds to TMPRSS4 comprises a VH comprising an HCDR1 comprising the amino acid sequence set forth in SEQ ID NO: 65, an HCDR2 comprising the amino acid sequence set forth in SEQ ID NO: 66 and an HCDR3 comprising the amino acid sequence set forth in SEQ ID NO: 67, and a VL comprising an LCDR1 comprising the amino acid sequence set forth in SEQ ID NO: 68, an LCDR2 comprising the amino acid sequence set forth in SEQ ID NO: 69, and an LCDR3 comprising the amino acid sequence set forth in SEQ ID NO: 70 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 63 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 64. In some embodiments, to TMPRSS4 comprises a VH comprising the amino acid sequence set forth in SEQ ID NO: 63 and a VL comprising the amino acid sequence set forth in SEQ ID NO: 64.

In some embodiments, the antibody or antigen-binding fragment that binds to human TMPRSS4 is or is derived from TMPRSS4 Ab10. In some embodiments, the antibody or antigen-binding fragment that binds to TMPRSS4 comprises a VH comprising an HCDR1 comprising the amino acid sequence set forth in SEQ ID NO: 34, an HCDR2 comprising the amino acid sequence set forth in SEQ ID NO: 73, and an HCDR3 comprising the amino acid sequence set forth in SEQ ID NO: 74, and a VL comprising an LCDR1 comprising the amino acid sequence set forth in SEQ ID NO: 75, an LCDR2 comprising the amino acid sequence set forth in SEQ ID NO: 38, and an LCDR3 comprising the amino acid sequence set forth in SEQ ID NO: 76 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 71 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 72. In some embodiments, to TMPRSS4 comprises a VH comprising the amino acid sequence set forth in SEQ ID NO: 71 and a VL comprising the amino acid sequence set forth in SEQ ID NO: 72.

In some embodiments, the antibody or antigen-binding fragment that binds to human TMPRSS4 is or is derived from TMPRSS4 Ab11. In some embodiments, the antibody or antigen-binding fragment that binds to TMPRSS4 comprises a VH comprising an HCDR1 comprising the amino acid sequence set forth in SEQ ID NO: 79, an HCDR2 comprising the amino acid sequence set forth in SEQ ID NO: 80, and an HCDR3 comprising the amino acid sequence set forth in SEQ ID NO: 81, and a VL comprising an LCDR1 comprising the amino acid sequence set forth in SEQ ID NO: 82, an LCDR2 comprising the amino acid sequence set forth in SEQ ID NO: 15, and an LCDR3 comprising the amino acid sequence set forth in SEQ ID NO: 83 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 77 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 78. In some embodiments, to TMPRSS4 comprises a VH comprising the amino acid sequence set forth in SEQ ID NO: 77 and a VL comprising the amino acid sequence set forth in SEQ ID NO: 78.

In some embodiments, the antibody or antigen-binding fragment that binds to human TMPRSS4 is or is derived from TMPRSS4 Ab12. In some embodiments, the antibody or antigen-binding fragment that binds to TMPRSS4 comprises a VH comprising an HCDR1 comprising the amino acid sequence set forth in SEQ ID NO: 86, an HCDR2 comprising the amino acid sequence set forth in SEQ ID NO: 87, and an HCDR3 comprising the amino acid sequence set forth in SEQ ID NO: 88, and a VL comprising an LCDR1 comprising the amino acid sequence set forth in SEQ ID NO: 89, an LCDR2 comprising the amino acid sequence set forth in SEQ ID NO: 90, and an LCDR3 comprising the amino acid sequence set forth in SEQ ID NO: 70 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 84 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 85. In some embodiments, to TMPRSS4 comprises a VH comprising the amino acid sequence set forth in SEQ ID NO: 84 and a VL comprising the amino acid sequence set forth in SEQ ID NO: 85.

In some embodiments, the antibody or antigen-binding fragment that binds to human TMPRSS4 is or is derived from TMPRSS4 Ab13. In some embodiments, the antibody or antigen-binding fragment that binds to TMPRSS4 comprises a VH comprising an HCDR1 comprising the amino acid sequence set forth in SEQ ID NO: 93, an HCDR2 comprising the amino acid sequence set forth in SEQ ID NO: 94, and an HCDR3 comprising the amino acid sequence set forth in SEQ ID NO: 95, and a VL comprising an LCDR1 comprising the amino acid sequence set forth in SEQ ID NO: 96, an LCDR2 comprising the amino acid sequence set forth in SEQ ID NO: 97, and an LCDR3 comprising the amino acid sequence set forth in SEQ ID NO: 98 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 91 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 92. In some embodiments, to TMPRSS4 comprises a VH comprising the amino acid sequence set forth in SEQ ID NO: 91 and a VL comprising the amino acid sequence set forth in SEQ ID NO: 92.

In some embodiments, the antibody or antigen-binding fragment that binds to human TMPRSS4 is or is derived from TMPRSS4 Ab14. In some embodiments, the antibody or antigen-binding fragment that binds to TMPRSS4 comprises a VH comprising an HCDR1 comprising the amino acid sequence set forth in SEQ ID NO: 101, an HCDR2 comprising the amino acid sequence set forth in SEQ ID NO: 102, and an HCDR3 comprising the amino acid sequence set forth in SEQ ID NO: 103, and a VL comprising an LCDR1 comprising the amino acid sequence set forth in SEQ ID NO: 104, an LCDR2 comprising the amino acid sequence set forth in SEQ ID NO: 105, and an LCDR3 comprising the amino acid sequence set forth in SEQ ID NO: 106 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 99 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 100. In some embodiments, to TMPRSS4 comprises a VH comprising the amino acid sequence set forth in SEQ ID NO: 99 and a VL comprising the amino acid sequence set forth in SEQ ID NO: 100.

In some embodiments, the antibody or antigen-binding fragment that binds to human TMPRSS4 is or is derived from TMPRSS4 Ab15. In some embodiments, the antibody or antigen-binding fragment that binds to TMPRSS4 comprises a VH comprising an HCDR1 comprising the amino acid sequence set forth in SEQ ID NO: 65, an HCDR2 comprising the amino acid sequence set forth in SEQ ID NO: 109, and an HCDR3 comprising the amino acid sequence set forth in SEQ ID NO: 110, and a VL comprising an LCDR1 comprising the amino acid sequence set forth in SEQ ID NO: 111, an LCDR2 comprising the amino acid sequence set forth in SEQ ID NO: 15, and an LCDR3 comprising the amino acid sequence set forth in SEQ ID NO: 112 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 107 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 108. In some embodiments, to TMPRSS4 comprises a VH comprising the amino acid sequence set forth in SEQ ID NO: 107 and a VL comprising the amino acid sequence set forth in SEQ ID NO: 108.

In some embodiments, the antibody or antigen-binding fragment that binds to human TMPRSS4 is or is derived from TMPRSS4 Ab16. In some embodiments, the antibody or antigen-binding fragment that binds to TMPRSS4 comprises a VH comprising an HCDR1 comprising the amino acid sequence set forth in SEQ ID NO: 115, an HCDR2 comprising the amino acid sequence set forth in SEQ ID NO: 80, and an HCDR3 comprising the amino acid sequence set forth in SEQ ID NO: 116, and a VL comprising an LCDR1 comprising the amino acid sequence set forth in SEQ ID NO: 117, an LCDR2 comprising the amino acid sequence set forth in SEQ ID NO: 118, and an LCDR3 comprising the amino acid sequence set forth in SEQ ID NO: 119 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 113 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 114. In some embodiments, to TMPRSS4 comprises a VH comprising the amino acid sequence set forth in SEQ ID NO: 113 and a VL comprising the amino acid sequence set forth in SEQ ID NO: 114.

In some embodiments, the antibody or antigen-binding fragment that binds to human TMPRSS4 is or is derived from TMPRSS4 Ab17. In some embodiments, the antibody or antigen-binding fragment that binds to TMPRSS4 comprises a VH comprising an HCDR1 comprising the amino acid sequence set forth in SEQ ID NO: 122, an HCDR2 comprising the amino acid sequence set forth in SEQ ID NO: 123, and an HCDR3 comprising the amino acid sequence set forth in SEQ ID NO: 124, and a VL comprising an LCDR1 comprising the amino acid sequence set forth in SEQ ID NO: 125, an LCDR2 comprising the amino acid sequence set forth in SEQ ID NO: 126, and an LCDR3 comprising the amino acid sequence set forth in SEQ ID NO: 127 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 120 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 121. In some embodiments, to TMPRSS4 comprises a VH comprising the amino acid sequence set forth in SEQ ID NO: 120 and a VL comprising the amino acid sequence set forth in SEQ ID NO: 121.

In some embodiments, the antibody or antigen-binding fragment that binds to human TMPRSS4 is or is derived from TMPRSS4 Ab18. In some embodiments, the antibody or antigen-binding fragment that binds to TMPRSS4 comprises a VH comprising an HCDR1 comprising the amino acid sequence set forth in SEQ ID NO: 130, an HCDR2 comprising the amino acid sequence set forth in SEQ ID NO: 131, and an HCDR3 comprising the amino acid sequence set forth in SEQ ID NO: 132, and a VL comprising an LCDR1 comprising the amino acid sequence set forth in SEQ ID NO: 44, an LCDR2 comprising the amino acid sequence set forth in SEQ ID NO: 45, and an LCDR3 comprising the amino acid sequence set forth in SEQ ID NO: 133 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 128 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 129. In some embodiments, to TMPRSS4 comprises a VH comprising the amino acid sequence set forth in SEQ ID NO: 128 and a VL comprising the amino acid sequence set forth in SEQ ID NO: 129.

In some embodiments, the antibody or antigen-binding fragment that binds to human TMPRSS4 is or is derived from TMPRSS4 Ab19. In some embodiments, the antibody or antigen-binding fragment that binds to TMPRSS4 comprises a VH comprising an HCDR1 comprising the amino acid sequence set forth in SEQ ID NO: 136, an HCDR2 comprising the amino acid sequence set forth in SEQ ID NO: 137, and an HCDR3 comprising the amino acid sequence set forth in SEQ ID NO: 138, and a VL comprising an LCDR1 comprising the amino acid sequence set forth in SEQ ID NO: 139, an LCDR2 comprising the amino acid sequence set forth in SEQ ID NO: 118, and an LCDR3 comprising the amino acid sequence set forth in SEQ ID NO: 70 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 134 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 135. In some embodiments, to TMPRSS4 comprises a VH comprising the amino acid sequence set forth in SEQ ID NO: 134 and a VL comprising the amino acid sequence set forth in SEQ ID NO: 135.

In some embodiments, the antibody or antigen-binding fragment that binds to human TMPRSS4 is or is derived from TMPRSS4 Ab20. In some embodiments, the antibody or antigen-binding fragment that binds to TMPRSS4 comprises a VH comprising an HCDR1 comprising the amino acid sequence set forth in SEQ ID NO: 65, an HCDR2 comprising the amino acid sequence set forth in SEQ ID NO: 142, and an HCDR3 comprising the amino acid sequence set forth in SEQ ID NO: 143, and a VL comprising an LCDR1 comprising the amino acid sequence set forth in SEQ ID NO: 144, an LCDR2 comprising the amino acid sequence set forth in SEQ ID NO: 145, and an LCDR3 comprising the amino acid sequence set forth in SEQ ID NO: 146 and/or the antibody or antigen-binding fragment that binds to TMPRSS4 comprises a VH comprising an amino acid sequence at least 80%, at least 85%, at least 90%, at least 91%, least 99%, at least 99.5% or 100% identical to the amino acid sequence set forth in SEQ ID NO: 140 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 141. In some embodiments, to TMPRSS4 comprises a VH comprising the amino acid sequence set forth in SEQ ID NO: 140 and a VL comprising the amino acid sequence set forth in SEQ ID NO: 141.

In some embodiments, the antibody or antigen-binding fragment that binds to human TMPRSS4 is or is derived from TMPRSS4 Ab21. In some embodiments, the antibody or antigen-binding fragment that binds to TMPRSS4 comprises a VH comprising an HCDR1 comprising the amino acid sequence set forth in SEQ ID NO: 149, an HCDR2 comprising the amino acid sequence set forth in SEQ ID NO: 150, and an HCDR3 comprising the amino acid sequence set forth in SEQ ID NO: 151, and a VL comprising an LCDR1 comprising the amino acid sequence set forth in SEQ ID NO: 139, an LCDR2 comprising the amino acid sequence set forth in SEQ ID NO: 30, and an LCDR3 comprising the amino acid sequence set forth in SEQ ID NO: 152 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 147 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 148. In some embodiments, to TMPRSS4 comprises a VH comprising the amino acid sequence set forth in SEQ ID NO: 147 and a VL comprising the amino acid sequence set forth in SEQ ID NO: 148.

In some embodiments, the antibody or antigen-binding fragment that binds to human TMPRSS4 is or is derived from TMPRSS4 Ab22. In some embodiments, the antibody or antigen-binding fragment that binds to TMPRSS4 comprises a VH comprising an HCDR1 comprising the amino acid sequence set forth in SEQ ID NO: 34, an HCDR2 comprising the amino acid sequence set forth in SEQ ID NO: 155, and an HCDR3 comprising the amino acid sequence set forth in SEQ ID NO: 156, and a VL comprising an LCDR1 comprising the amino acid sequence set forth in SEQ ID NO: 157, an LCDR2 comprising the amino acid sequence set forth in SEQ ID NO: 15, and an LCDR3 comprising the amino acid sequence set forth in SEQ ID NO: 158 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 153 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 154. In some embodiments, to TMPRSS4 comprises a VH comprising the amino acid sequence set forth in SEQ ID NO: 153 and a VL comprising the amino acid sequence set forth in SEQ ID NO: 154.

In some embodiments, the antibody or antigen-binding fragment that binds to human TMPRSS4 is or is derived from TMPRSS4 Ab23. In some embodiments, the antibody or antigen-binding fragment that binds to TMPRSS4 comprises a VH comprising an HCDR1 comprising the amino acid sequence set forth in SEQ ID NO: 161, an HCDR2 comprising the amino acid sequence set forth in SEQ ID NO: 162, and an HCDR3 comprising the amino acid sequence set forth in SEQ ID NO: 163, and a VL comprising an LCDR1 comprising the amino acid sequence set forth in SEQ ID NO: 164, an LCDR2 comprising the amino acid sequence set forth in SEQ ID NO: 38, and an LCDR3 comprising the amino acid sequence set forth in SEQ ID NO: 165 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 159 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 160. In some embodiments, to TMPRSS4 comprises a VH comprising the amino acid sequence set forth in SEQ ID NO: 159 and a VL comprising the amino acid sequence set forth in SEQ ID NO: 160.

In some embodiments, the antibody or antigen-binding fragment that binds to human TMPRSS4 is or is derived from TMPRSS4 Ab24. In some embodiments, the antibody or antigen-binding fragment that binds to TMPRSS4 comprises a VH comprising an HCDR1 comprising the amino acid sequence set forth in SEQ ID NO: 34, an HCDR2 comprising the amino acid sequence set forth in SEQ ID NO: 168, and an HCDR3 comprising the amino acid sequence set forth in SEQ ID NO: 169, and a VL comprising an LCDR1 comprising the amino acid sequence set forth in SEQ ID NO: 60, an LCDR2 comprising the amino acid sequence set forth in SEQ ID NO: 30, and an LCDR3 comprising the amino acid sequence set forth in SEQ ID NO: 170 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 166 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 167. In some embodiments, to TMPRSS4 comprises a VH comprising the amino acid sequence set forth in SEQ ID NO: 166 and a VL comprising the amino acid sequence set forth in SEQ ID NO: 167.

In some embodiments, the antibody or antigen-binding fragment that binds to human TMPRSS4 is or is derived from TMPRSS4 Ab25. In some embodiments, the antibody or antigen-binding fragment that binds to TMPRSS4 comprises a VH comprising an HCDR1 comprising the amino acid sequence set forth in SEQ ID NO: 173, an HCDR2 comprising the amino acid sequence set forth in SEQ ID NO: 66, and an HCDR3 comprising the amino acid sequence set forth in SEQ ID NO: 174, and a VL comprising an LCDR1 comprising the amino acid sequence set forth in SEQ ID NO: 175, an LCDR2 comprising the amino acid sequence set forth in SEQ ID NO: 176, and an LCDR3 comprising the amino acid sequence set forth in SEQ ID NO: 177 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 171 and a VL comprising an amino acid sequence at least 80%, at least 85%, at least 90%, at least 98%, at least 99%, at least 99.5% or 100% identical to the amino acid sequence set forth in SEQ ID NO: 172. In some embodiments, to TMPRSS4 comprises a VH comprising the amino acid sequence set forth in SEQ ID NO: 171 and a VL comprising the amino acid sequence set forth in SEQ ID NO: 172.

In some embodiments, the antibody or antigen-binding fragment that binds to human TMPRSS4 is or is derived from TMPRSS4 Ab26. In some embodiments, the antibody or antigen-binding fragment that binds to TMPRSS4 comprises a VH comprising an HCDR1 comprising the amino acid sequence set forth in SEQ ID NO: 180, an HCDR2 comprising the amino acid sequence set forth in SEQ ID NO: 181, and an HCDR3 comprising the amino acid sequence set forth in SEQ ID NO: 182, and a VL comprising an LCDR1 comprising the amino acid sequence set forth in SEQ ID NO: 183, an LCDR2 comprising the amino acid sequence set forth in SEQ ID NO: 15, and an LCDR3 comprising the amino acid sequence set forth in SEQ ID NO: 184 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 178 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 179. In some embodiments, to TMPRSS4 comprises a VH comprising the amino acid sequence set forth in SEQ ID NO: 178 and a VL comprising the amino acid sequence set forth in SEQ ID NO: 179.

In some embodiments, the antibody or antigen-binding fragment that binds to human TMPRSS4 is or is derived from TMPRSS4 Ab27. In some embodiments, the antibody or antigen-binding fragment that binds to TMPRSS4 comprises a VH comprising an HCDR1 comprising the amino acid sequence set forth in SEQ ID NO: 187, an HCDR2 comprising the amino acid sequence set forth in SEQ ID NO: 188, and an HCDR3 comprising the amino acid sequence set forth in SEQ ID NO: 189, and a VL comprising an LCDR1 comprising the amino acid sequence set forth in SEQ ID NO: 292, an LCDR2 comprising the amino acid sequence set forth in SEQ ID NO: 15, and an LCDR3 comprising the amino acid sequence set forth in SEQ ID NO: 190 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 185 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 186. In some embodiments, to TMPRSS4 comprises a VH comprising the amino acid sequence set forth in SEQ ID NO: 185 and a VL comprising the amino acid sequence set forth in SEQ ID NO: 186.

In some embodiments, the antibody or antigen-binding fragment that binds to human TMPRSS4 is or is derived from TMPRSS4 Ab28. In some embodiments, the antibody or antigen-binding fragment that binds to TMPRSS4 comprises a VH comprising an HCDR1 comprising the amino acid sequence set forth in SEQ ID NO: 193, an HCDR2 comprising the amino acid sequence set forth in SEQ ID NO: 194, and an HCDR3 comprising the amino acid sequence set forth in SEQ ID NO: 195, and a VL comprising an LCDR1 comprising the amino acid sequence set forth in SEQ ID NO: 196, an LCDR2 comprising the amino acid sequence set forth in SEQ ID NO: 197, and an LCDR3 comprising the amino acid sequence set forth in SEQ ID NO: 198 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 191 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 192. In some embodiments, to TMPRSS4 comprises a VH comprising the amino acid sequence set forth in SEQ ID NO: 191 and a VL comprising the amino acid sequence set forth in SEQ ID NO: 192.

In some embodiments, the antibody or antigen-binding fragment that binds to human TMPRSS4 is or is derived from TMPRSS4 Ab29. In some embodiments, the antibody or antigen-binding fragment that binds to TMPRSS4 comprises a VH comprising an HCDR1 comprising the amino acid sequence set forth in SEQ ID NO: 201, an HCDR2 comprising the amino acid sequence set forth in SEQ ID NO: 194, and an HCDR3 comprising the amino acid sequence set forth in SEQ ID NO: 202, and a VL comprising an LCDR1 comprising the amino acid sequence set forth in SEQ ID NO: 29, an LCDR2 comprising the amino acid sequence set forth in SEQ ID NO: 203, and an LCDR3 comprising the amino acid sequence set forth in SEQ ID NO: 204 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 199 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 200. In some embodiments, to TMPRSS4 comprises a VH comprising the amino acid sequence set forth in SEQ ID NO: 199 and a VL comprising the amino acid sequence set forth in SEQ ID NO: 200.

In some embodiments, the antibody or antigen-binding fragment that binds to human TMPRSS4 is or is derived from TMPRSS4 Ab30. In some embodiments, the antibody or antigen-binding fragment that binds to TMPRSS4 comprises a VH comprising an HCDR1 comprising the amino acid sequence set forth in SEQ ID NO: 207, an HCDR2 comprising the amino acid sequence set forth in SEQ ID NO: 194, and an HCDR3 comprising the amino acid sequence set forth in SEQ ID NO: 208, and a VL comprising an LCDR1 comprising the amino acid sequence set forth in SEQ ID NO: 209, an LCDR2 comprising the amino acid sequence set forth in SEQ ID NO: 38, and an LCDR3 comprising the amino acid sequence set forth in SEQ ID NO: 210 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 205 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 206. In some embodiments, to TMPRSS4 comprises a VH comprising the amino acid sequence set forth in SEQ ID NO: 205 and a VL comprising the amino acid sequence set forth in SEQ ID NO: 206.

In some embodiments, the antibody or antigen-binding fragment that binds to human TMPRSS4 is or is derived from TMPRSS4 Ab31. In some embodiments, the antibody or antigen-binding fragment that binds to TMPRSS4 comprises a VH comprising an HCDR1 comprising the amino acid sequence set forth in SEQ ID NO: 213, an HCDR2 comprising the amino acid sequence set forth in SEQ ID NO: 214, and an HCDR3 comprising the amino acid sequence set forth in SEQ ID NO: 215, and a VL comprising an LCDR1 comprising the amino acid sequence set forth in SEQ ID NO: 216, an LCDR2 comprising the amino acid sequence set forth in SEQ ID NO: 38, and an LCDR3 comprising the amino acid sequence set forth in SEQ ID NO: 217 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 211 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 212. In some embodiments, to TMPRSS4 comprises a VH comprising the amino acid sequence set forth in SEQ ID NO: 211 and a VL comprising the amino acid sequence set forth in SEQ ID NO: 212.

In some embodiments, the antibody or antigen-binding fragment that binds to human TMPRSS4 is or is derived from TMPRSS4 Ab32. In some embodiments, the antibody or antigen-binding fragment that binds to TMPRSS4 comprises a VH comprising an HCDR1 comprising the amino acid sequence set forth in SEQ ID NO: 220, an HCDR2 comprising the amino acid sequence set forth in SEQ ID NO: 221, and an HCDR3 comprising the amino acid sequence set forth in SEQ ID NO: 81, and a VL comprising an LCDR1 comprising the amino acid sequence set forth in SEQ ID NO: 222, an LCDR2 comprising the amino acid sequence set forth in SEQ ID NO: 223, and an LCDR3 comprising the amino acid sequence set forth in SEQ ID NO: 224 and/or the antibody or antigen-binding fragment that binds to TMPRSS4 comprises a VH comprising an amino acid sequence at least 80%, at least 85%, at least 90%, at least 91%, least 99%, at least 99.5% or 100% identical to the amino acid sequence set forth in SEQ ID NO: 218 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 219. In some embodiments, to TMPRSS4 comprises a VH comprising the amino acid sequence set forth in SEQ ID NO: 218 and a VL comprising the amino acid sequence set forth in SEQ ID NO: 219.

In some embodiments, the antibody or antigen-binding fragment that binds to human TMPRSS4 is or is derived from TMPRSS4 Ab33. In some embodiments, the antibody or antigen-binding fragment that binds to TMPRSS4 comprises a VH comprising an HCDR1 comprising the amino acid sequence set forth in SEQ ID NO: 227, an HCDR2 comprising the amino acid sequence set forth in SEQ ID NO: 228, and an HCDR3 comprising the amino acid sequence set forth in SEQ ID NO: 229, and a VL comprising an LCDR1 comprising the amino acid sequence set forth in SEQ ID NO: 230, an LCDR2 comprising the amino acid sequence set forth in SEQ ID NO: 231, and an LCDR3 comprising the amino acid sequence set forth in SEQ ID NO: 232 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 225 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 226. In some embodiments, to TMPRSS4 comprises a VH comprising the amino acid sequence set forth in SEQ ID NO: 225 and a VL comprising the amino acid sequence set forth in SEQ ID NO: 226.

In some embodiments, the antibody or antigen-binding fragment that binds to human TMPRSS4 is or is derived from TMPRSS4 Ab34. In some embodiments, the antibody or antigen-binding fragment that binds to TMPRSS4 comprises a VH comprising an HCDR1 comprising the amino acid sequence set forth in SEQ ID NO: 235, an HCDR2 comprising the amino acid sequence set forth in SEQ ID NO: 236, and an HCDR3 comprising the amino acid sequence set forth in SEQ ID NO: 237, and a VL comprising an LCDR1 comprising the amino acid sequence set forth in SEQ ID NO: 238, an LCDR2 comprising the amino acid sequence set forth in SEQ ID NO: 239, and an LCDR3 comprising the amino acid sequence set forth in SEQ ID NO: 240 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 233 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 234. In some embodiments, to TMPRSS4 comprises a VH comprising the amino acid sequence set forth in SEQ ID NO: 233 and a VL comprising the amino acid sequence set forth in SEQ ID NO: 234.

In some embodiments, the antibody or antigen-binding fragment that binds to human TMPRSS4 is or is derived from TMPRSS4 Ab35. In some embodiments, the antibody or antigen-binding fragment that binds to TMPRSS4 comprises a VH comprising an HCDR1 comprising the amino acid sequence set forth in SEQ ID NO: 193, an HCDR2 comprising the amino acid sequence set forth in SEQ ID NO: 243, and an HCDR3 comprising the amino acid sequence set forth in SEQ ID NO: 244, and a VL comprising an LCDR1 comprising the amino acid sequence set forth in SEQ ID NO: 6, an LCDR2 comprising the amino acid sequence set forth in SEQ ID NO: 7, and an LCDR3 comprising the amino acid sequence set forth in SEQ ID NO: 245 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 241 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 242. In some embodiments, to TMPRSS4 comprises a VH comprising the amino acid sequence set forth in SEQ ID NO: 241 and a VL comprising the amino acid sequence set forth in SEQ ID NO: 242.

In some embodiments, the antibody or antigen-binding fragment that binds to human TMPRSS4 is or is derived from TMPRSS4 Ab36. In some embodiments, the antibody or antigen-binding fragment that binds to TMPRSS4 comprises a VH comprising an HCDR1 comprising the amino acid sequence set forth in SEQ ID NO: 248, an HCDR2 comprising the amino acid sequence set forth in SEQ ID NO: 249, and an HCDR3 comprising the amino acid sequence set forth in SEQ ID NO: 250, and a VL comprising an LCDR1 comprising the amino acid sequence set forth in SEQ ID NO: 292, an LCDR2 comprising the amino acid sequence set forth in SEQ ID NO: 251, and an LCDR3 comprising the amino acid sequence set forth in SEQ ID NO: 252 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 246 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 247. In some embodiments, to TMPRSS4 comprises a VH comprising the amino acid sequence set forth in SEQ ID NO: 246 and a VL comprising the amino acid sequence set forth in SEQ ID NO: 247.

In some embodiments, the antibody or antigen-binding fragment that binds to human TMPRSS4 is or is derived from TMPRSS4 Ab37. In some embodiments, the antibody or antigen-binding fragment that binds to TMPRSS4 comprises a VH comprising an HCDR1 comprising the amino acid sequence set forth in SEQ ID NO: 180, an HCDR2 comprising the amino acid sequence set forth in SEQ ID NO: 255, and an HCDR3 comprising the amino acid sequence set forth in SEQ ID NO: 256, and a VL comprising an LCDR1 comprising the amino acid sequence set forth in SEQ ID NO: 139, an LCDR2 comprising the amino acid sequence set forth in SEQ ID NO: 257, and an LCDR3 comprising the amino acid sequence set forth in SEQ ID NO: 258 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 253 and a VL comprising an amino acid sequence at least 80%, at least 85%, at least 90%, at least 98%, at least 99%, at least 99.5% or 100% identical to the amino acid sequence set forth in SEQ ID NO: 254. In some embodiments, to TMPRSS4 comprises a VH comprising the amino acid sequence set forth in SEQ ID NO: 253 and a VL comprising the amino acid sequence set forth in SEQ ID NO: 254.

In some embodiments, the antibody or antigen-binding fragment that binds to human TMPRSS4 is or is derived from TMPRSS4 Ab38. In some embodiments, the antibody or antigen-binding fragment that binds to TMPRSS4 comprises a VH comprising an HCDR1 comprising the amino acid sequence set forth in SEQ ID NO: 261, an HCDR2 comprising the amino acid sequence set forth in SEQ ID NO: 50, and an HCDR3 comprising the amino acid sequence set forth in SEQ ID NO: 262, and a VL comprising an LCDR1 comprising the amino acid sequence set forth in SEQ ID NO: 263, an LCDR2 comprising the amino acid sequence set forth in SEQ ID NO: 264, and an LCDR3 comprising the amino acid sequence set forth in SEQ ID NO: 265 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 259 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 260. In some embodiments, to TMPRSS4 comprises a VH comprising the amino acid sequence set forth in SEQ ID NO: 259 and a VL comprising the amino acid sequence set forth in SEQ ID NO: 260.

In some embodiments, the antibody or antigen-binding fragment that binds to human TMPRSS4 is or is derived from TMPRSS4 Ab39. In some embodiments, the antibody or antigen-binding fragment that binds to TMPRSS4 comprises a VH comprising an HCDR1 comprising the amino acid sequence set forth in SEQ ID NO: 268, an HCDR2 comprising the amino acid sequence set forth in SEQ ID NO: 269, and an HCDR3 comprising the amino acid sequence set forth in SEQ ID NO: 270, and a VL comprising an LCDR1 comprising the amino acid sequence set forth in SEQ ID NO: 271, an LCDR2 comprising the amino acid sequence set forth in SEQ ID NO: 272, and an LCDR3 comprising the amino acid sequence set forth in SEQ ID NO: 273 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 266 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 267. In some embodiments, to TMPRSS4 comprises a VH comprising the amino acid sequence set forth in SEQ ID NO: 266 and a VL comprising the amino acid sequence set forth in SEQ ID NO: 267.

In some embodiments, the antibody or antigen-binding fragment that binds to human TMPRSS4 is or is derived from TMPRSS4 Ab40. In some embodiments, the antibody or antigen-binding fragment that binds to TMPRSS4 comprises a VH comprising an HCDR1 comprising the amino acid sequence set forth in SEQ ID NO: 220, an HCDR2 comprising the amino acid sequence set forth in SEQ ID NO: 276, and an HCDR3 comprising the amino acid sequence set forth in SEQ ID NO: 277, and a VL comprising an LCDR1 comprising the amino acid sequence set forth in SEQ ID NO: 278, an LCDR2 comprising the amino acid sequence set forth in SEQ ID NO: 279, and an LCDR3 comprising the amino acid sequence set forth in SEQ ID NO: 280 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 274 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 275. In some embodiments, to TMPRSS4 comprises a VH comprising the amino acid sequence set forth in SEQ ID NO: 274 and a VL comprising the amino acid sequence set forth in SEQ ID NO: 275.

In some embodiments, the antibody or antigen-binding fragment that binds to human TMPRSS4 is or is derived from TMPRSS4 Ab41. In some embodiments, the antibody or antigen-binding fragment that binds to TMPRSS4 comprises a VH comprising an HCDR1 comprising the amino acid sequence set forth in SEQ ID NO: 220, an HCDR2 comprising the amino acid sequence set forth in SEQ ID NO: 283, and an HCDR3 comprising the amino acid sequence set forth in SEQ ID NO: 277, and a VL comprising an LCDR1 comprising the amino acid sequence set forth in SEQ ID NO: 284, an LCDR2 comprising the amino acid sequence set forth in SEQ ID NO: 239, and an LCDR3 comprising the amino acid sequence set forth in SEQ ID NO: 285 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 281 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 282. In some embodiments, to TMPRSS4 comprises a VH comprising the amino acid sequence set forth in SEQ ID NO: 281 and a VL comprising the amino acid sequence set forth in SEQ ID NO: 282.

In some embodiments, the antibody or antigen-binding fragment that binds to human TMPRSS4 is or is derived from TMPRSS4 Ab42. In some embodiments, the antibody or antigen-binding fragment that binds to TMPRSS4 comprises a VH comprising an HCDR1 comprising the amino acid sequence set forth in SEQ ID NO: 288, an HCDR2 comprising the amino acid sequence set forth in SEQ ID NO: 194, and an HCDR3 comprising the amino acid sequence set forth in SEQ ID NO: 289, and a VL comprising an LCDR1 comprising the amino acid sequence set forth in SEQ ID NO: 290, an LCDR2 comprising the amino acid sequence set forth in SEQ ID NO: 291, and an LCDR3 comprising the amino acid sequence set forth in SEQ ID NO: 293 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 286 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 287. In some embodiments, to TMPRSS4 comprises a VH comprising the amino acid sequence set forth in SEQ ID NO: 286 and a VL comprising the amino acid sequence set forth in SEQ ID NO: 287.

In some embodiments, the antibody or antigen-binding fragment that binds to human TMPRSS4 is or is derived from TMPRSS4 Ab43. In some embodiments, the antibody or antigen-binding fragment that binds to TMPRSS4 comprises a VH comprising an HCDR1 comprising the amino acid sequence set forth in SEQ ID NO: 296, an HCDR2 comprising the amino acid sequence set forth in SEQ ID NO: 297, and an HCDR3 comprising the amino acid sequence set forth in SEQ ID NO: 298, and a VL comprising an LCDR1 comprising the amino acid sequence set forth in SEQ ID NO: 6, an LCDR2 comprising the amino acid sequence set forth in SEQ ID NO: 15, and an LCDR3 comprising the amino acid sequence set forth in SEQ ID NO: 152 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 294 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 295. In some embodiments, to TMPRSS4 comprises a VH comprising the amino acid sequence set forth in SEQ ID NO: 294 and a VL comprising the amino acid sequence set forth in SEQ ID NO: 295.

In some embodiments, the antibody or antigen-binding fragment that binds to human TMPRSS4 is or is derived from TMPRSS4 Ab44. In some embodiments, the antibody or antigen-binding fragment that binds to TMPRSS4 comprises a VH comprising an HCDR1 comprising the amino acid sequence set forth in SEQ ID NO: 301, an HCDR2 comprising the amino acid sequence set forth in SEQ ID NO: 80, and an HCDR3 comprising the amino acid sequence set forth in SEQ ID NO: 302, and a VL comprising an LCDR1 comprising the amino acid sequence set forth in SEQ ID NO: 6, an LCDR2 comprising the amino acid sequence set forth in SEQ ID NO: 303, and an LCDR3 comprising the amino acid sequence set forth in SEQ ID NO: 304 and/or the antibody or antigen-binding fragment that binds to TMPRSS4 comprises a VH comprising an amino acid sequence at least 80%, at least 85%, at least 90%, at least 91%, least 99%, at least 99.5% or 100% identical to the amino acid sequence set forth in SEQ ID NO: 299 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 300. In some embodiments, to TMPRSS4 comprises a VH comprising the amino acid sequence set forth in SEQ ID NO: 299 and a VL comprising the amino acid sequence set forth in SEQ ID NO: 300.

In some embodiments, the antibody or antigen-binding fragment that binds to human TMPRSS4 is or is derived from TMPRSS4 Ab45. In some embodiments, the antibody or antigen-binding fragment that binds to TMPRSS4 comprises a VH comprising an HCDR1 comprising the amino acid sequence set forth in SEQ ID NO: 307, an HCDR2 comprising the amino acid sequence set forth in SEQ ID NO: 80, and an HCDR3 comprising the amino acid sequence set forth in SEQ ID NO: 308, and a VL comprising an LCDR1 comprising the amino acid sequence set forth in SEQ ID NO: 309, an LCDR2 comprising the amino acid sequence set forth in SEQ ID NO: 310, and an LCDR3 comprising the amino acid sequence set forth in SEQ ID NO: 311 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 305 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 306. In some embodiments, to TMPRSS4 comprises a VH comprising the amino acid sequence set forth in SEQ ID NO: 305 and a VL comprising the amino acid sequence set forth in SEQ ID NO: 306.

In some embodiments, the antibody or antigen-binding fragment that binds to human TMPRSS4 is or is derived from TMPRSS4 Ab46. In some embodiments, the antibody or antigen-binding fragment that binds to TMPRSS4 comprises a VH comprising an HCDR1 comprising the amino acid sequence set forth in SEQ ID NO: 49, an HCDR2 comprising the amino acid sequence set forth in SEQ ID NO: 66, and an HCDR3 comprising the amino acid sequence set forth in SEQ ID NO: 110, and a VL comprising an LCDR1 comprising the amino acid sequence set forth in SEQ ID NO: 292, an LCDR2 comprising the amino acid sequence set forth in SEQ ID NO: 15, and an LCDR3 comprising the amino acid sequence set forth in SEQ ID NO: 314 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 312 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 313. In some embodiments, to TMPRSS4 comprises a VH comprising the amino acid sequence set forth in SEQ ID NO: 312 and a VL comprising the amino acid sequence set forth in SEQ ID NO: 313.

In some embodiments, the antibody or antigen-binding fragment that binds to human TMPRSS4 is or is derived from TMPRSS4 Ab47. In some embodiments, the antibody or antigen-binding fragment that binds to TMPRSS4 comprises a VH comprising an HCDR1 comprising the amino acid sequence set forth in SEQ ID NO: 317, an HCDR2 comprising the amino acid sequence set forth in SEQ ID NO: 194, and an HCDR3 comprising the amino acid sequence set forth in SEQ ID NO: 318, and a VL comprising an LCDR1 comprising the amino acid sequence set forth in SEQ ID NO: 278, an LCDR2 comprising the amino acid sequence set forth in SEQ ID NO: 15, and an LCDR3 comprising the amino acid sequence set forth in SEQ ID NO: 252 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 315 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 316. In some embodiments, to TMPRSS4 comprises a VH comprising the amino acid sequence set forth in SEQ ID NO: 315 and a VL comprising the amino acid sequence set forth in SEQ ID NO: 316.

In some embodiments, the antibody or antigen-binding fragment that binds to human TMPRSS4 is or is derived from TMPRSS4 Ab48. In some embodiments, the antibody or antigen-binding fragment that binds to TMPRSS4 comprises a VH comprising an HCDR1 comprising the amino acid sequence set forth in SEQ ID NO: 321, an HCDR2 comprising the amino acid sequence set forth in SEQ ID NO: 322, and an HCDR3 comprising the amino acid sequence set forth in SEQ ID NO: 323, and a VL comprising an LCDR1 comprising the amino acid sequence set forth in SEQ ID NO: 324, an LCDR2 comprising the amino acid sequence set forth in SEQ ID NO: 325, and an LCDR3 comprising the amino acid sequence set forth in SEQ ID NO: 16 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 319 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 320. In some embodiments, to TMPRSS4 comprises a VH comprising the amino acid sequence set forth in SEQ ID NO: 319 and a VL comprising the amino acid sequence set forth in SEQ ID NO: 320.

In some embodiments, the antibody or antigen-binding fragment that binds to human TMPRSS4 is or is derived from TMPRSS4 Ab49. In some embodiments, the antibody or antigen-binding fragment that binds to TMPRSS4 comprises a VH comprising an HCDR1 comprising the amino acid sequence set forth in SEQ ID NO: 193, an HCDR2 comprising the amino acid sequence set forth in SEQ ID NO: 80, and an HCDR3 comprising the amino acid sequence set forth in SEQ ID NO: 328, and a VL comprising an LCDR1 comprising the amino acid sequence set forth in SEQ ID NO: 329, an LCDR2 comprising the amino acid sequence set forth in SEQ ID NO: 330, and an LCDR3 comprising the amino acid sequence set forth in SEQ ID NO: 331 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 326 and a VL comprising an amino acid sequence at least 80%, at least 85%, at least 90%, at least 98%, at least 99%, at least 99.5% or 100% identical to the amino acid sequence set forth in SEQ ID NO: 327. In some embodiments, to TMPRSS4 comprises a VH comprising the amino acid sequence set forth in SEQ ID NO: 326 and a VL comprising the amino acid sequence set forth in SEQ ID NO: 327.

In some embodiments, the antibody or antigen-binding fragment that binds to human TMPRSS4 is or is derived from TMPRSS4 Ab50. In some embodiments, the antibody or antigen-binding fragment that binds to TMPRSS4 comprises a VH comprising an HCDR1 comprising the amino acid sequence set forth in SEQ ID NO: 193, an HCDR2 comprising the amino acid sequence set forth in SEQ ID NO: 334, and an HCDR3 comprising the amino acid sequence set forth in SEQ ID NO: 110, and a VL comprising an LCDR1 comprising the amino acid sequence set forth in SEQ ID NO: 324, an LCDR2 comprising the amino acid sequence set forth in SEQ ID NO: 335, and an LCDR3 comprising the amino acid sequence set forth in SEQ ID NO: 336 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 332 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 333. In some embodiments, to TMPRSS4 comprises a VH comprising the amino acid sequence set forth in SEQ ID NO: 332 and a VL comprising the amino acid sequence set forth in SEQ ID NO: 333.

In some embodiments, the antibody or antigen-binding fragment that binds to human TMPRSS4 is or is derived from TMPRSS4 Ab51. In some embodiments, the antibody or antigen-binding fragment that binds to TMPRSS4 comprises a VH comprising an HCDR1 comprising the amino acid sequence set forth in SEQ ID NO: 93, an HCDR2 comprising the amino acid sequence set forth in SEQ ID NO: 338, and an HCDR3 comprising the amino acid sequence set forth in SEQ ID NO: 339, and a VL comprising an LCDR1 comprising the amino acid sequence set forth in SEQ ID NO: 278, an LCDR2 comprising the amino acid sequence set forth in SEQ ID NO: 15, and an LCDR3 comprising the amino acid sequence set forth in SEQ ID NO: 252 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 337 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 316. In some embodiments, to TMPRSS4 comprises a VH comprising the amino acid sequence set forth in SEQ ID NO: 337 and a VL comprising the amino acid sequence set forth in SEQ ID NO: 316.

In some embodiments, the antibody or antigen-binding fragment that binds to human TMPRSS4 is or is derived from TMPRSS4 Ab52. In some embodiments, the antibody or antigen-binding fragment that binds to TMPRSS4 comprises a VH comprising an HCDR1 comprising the amino acid sequence set forth in SEQ ID NO: 342, an HCDR2 comprising the amino acid sequence set forth in SEQ ID NO: 343, and an HCDR3 comprising the amino acid sequence set forth in SEQ ID NO: 344, and a VL comprising an LCDR1 comprising the amino acid sequence set forth in SEQ ID NO: 345, an LCDR2 comprising the amino acid sequence set forth in SEQ ID NO: 90, and an LCDR3 comprising the amino acid sequence set forth in SEQ ID NO: 346 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 340 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 341. In some embodiments, to TMPRSS4 comprises a VH comprising the amino acid sequence set forth in SEQ ID NO: 340 and a VL comprising the amino acid sequence set forth in SEQ ID NO: 341.

In some embodiments, the antibody or antigen-binding fragment that binds to human TMPRSS4 is or is derived from TMPRSS4 Ab53. In some embodiments, the antibody or antigen-binding fragment that binds to TMPRSS4 comprises a VH comprising an HCDR1 comprising the amino acid sequence set forth in SEQ ID NO: 65, an HCDR2 comprising the amino acid sequence set forth in SEQ ID NO: 349, and an HCDR3 comprising the amino acid sequence set forth in SEQ ID NO: 229, and a VL comprising an LCDR1 comprising the amino acid sequence set forth in SEQ ID NO: 350, an LCDR2 comprising the amino acid sequence set forth in SEQ ID NO: 351, and an LCDR3 comprising the amino acid sequence set forth in SEQ ID NO: 352 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 347 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 348. In some embodiments, to TMPRSS4 comprises a VH comprising the amino acid sequence set forth in SEQ ID NO: 347 and a VL comprising the amino acid sequence set forth in SEQ ID NO: 348.

In some embodiments, the antibody or antigen-binding fragment that binds to human TMPRSS4 is or is derived from TMPRSS4 Ab54. In some embodiments, the antibody or antigen-binding fragment that binds to TMPRSS4 comprises a VH comprising an HCDR1 comprising the amino acid sequence set forth in SEQ ID NO: 355, an HCDR2 comprising the amino acid sequence set forth in SEQ ID NO: 194, and an HCDR3 comprising the amino acid sequence set forth in SEQ ID NO: 110, and a VL comprising an LCDR1 comprising the amino acid sequence set forth in SEQ ID NO: 324, an LCDR2 comprising the amino acid sequence set forth in SEQ ID NO: 15, and an LCDR3 comprising the amino acid sequence set forth in SEQ ID NO: 280 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 353 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 354. In some embodiments, to TMPRSS4 comprises a VH comprising the amino acid sequence set forth in SEQ ID NO: 353 and a VL comprising the amino acid sequence set forth in SEQ ID NO: 354.

In some embodiments, the antibody or antigen-binding fragment that binds to human TMPRSS4 is or is derived from TMPRSS4 Ab55. In some embodiments, the antibody or antigen-binding fragment that binds to TMPRSS4 comprises a VH comprising an HCDR1 comprising the amino acid sequence set forth in SEQ ID NO: 207, an HCDR2 comprising the amino acid sequence set forth in SEQ ID NO: 58, and an HCDR3 comprising the amino acid sequence set forth in SEQ ID NO: 358, and a VL comprising an LCDR1 comprising the amino acid sequence set forth in SEQ ID NO: 60, an LCDR2 comprising the amino acid sequence set forth in SEQ ID NO: 15, and an LCDR3 comprising the amino acid sequence set forth in SEQ ID NO: 359 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 356 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 357. In some embodiments, to TMPRSS4 comprises a VH comprising the amino acid sequence set forth in SEQ ID NO: 356 and a VL comprising the amino acid sequence set forth in SEQ ID NO: 357.

In some embodiments, the antibody or antigen-binding fragment that binds to human TMPRSS4 is or is derived from TMPRSS4 Ab56. In some embodiments, the antibody or antigen-binding fragment that binds to TMPRSS4 comprises a VH comprising an HCDR1 comprising the amino acid sequence set forth in SEQ ID NO: 362, an HCDR2 comprising the amino acid sequence set forth in SEQ ID NO: 363, and an HCDR3 comprising the amino acid sequence set forth in SEQ ID NO: 364, and a VL comprising an LCDR1 comprising the amino acid sequence set forth in SEQ ID NO: 29, an LCDR2 comprising the amino acid sequence set forth in SEQ ID NO: 203, and an LCDR3 comprising the amino acid sequence set forth in SEQ ID NO: 365 and/or the antibody or antigen-binding fragment that binds to TMPRSS4 comprises a VH comprising an amino acid sequence at least 80%, at least 85%, at least 90%, at least 91%, least 99%, at least 99.5% or 100% identical to the amino acid sequence set forth in SEQ ID NO: 360 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 361. In some embodiments, to TMPRSS4 comprises a VH comprising the amino acid sequence set forth in SEQ ID NO: 360 and a VL comprising the amino acid sequence set forth in SEQ ID NO: 361.

In some embodiments, the antibody or antigen-binding fragment that binds to human TMPRSS4 is or is derived from TMPRSS4 Ab57. In some embodiments, the antibody or antigen-binding fragment that binds to TMPRSS4 comprises a VH comprising an HCDR1 comprising the amino acid sequence set forth in SEQ ID NO: 317, an HCDR2 comprising the amino acid sequence set forth in SEQ ID NO: 80, and an HCDR3 comprising the amino acid sequence set forth in SEQ ID NO: 368, and a VL comprising an LCDR1 comprising the amino acid sequence set forth in SEQ ID NO: 29, an LCDR2 comprising the amino acid sequence set forth in SEQ ID NO: 203, and an LCDR3 comprising the amino acid sequence set forth in SEQ ID NO: 369 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 366 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 367. In some embodiments, to TMPRSS4 comprises a VH comprising the amino acid sequence set forth in SEQ ID NO: 366 and a VL comprising the amino acid sequence set forth in SEQ ID NO: 367.

In some embodiments, the antibody or antigen-binding fragment that binds to human TMPRSS4 is or is derived from TMPRSS4 Ab58. In some embodiments, the antibody or antigen-binding fragment that binds to TMPRSS4 comprises a VH comprising an HCDR1 comprising the amino acid sequence set forth in SEQ ID NO: 372, an HCDR2 comprising the amino acid sequence set forth in SEQ ID NO: 373, and an HCDR3 comprising the amino acid sequence set forth in SEQ ID NO: 374, and a VL comprising an LCDR1 comprising the amino acid sequence set forth in SEQ ID NO: 29, an LCDR2 comprising the amino acid sequence set forth in SEQ ID NO: 203, and an LCDR3 comprising the amino acid sequence set forth in SEQ ID NO: 375 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 370 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 371. In some embodiments, to TMPRSS4 comprises a VH comprising the amino acid sequence set forth in SEQ ID NO: 370 and a VL comprising the amino acid sequence set forth in SEQ ID NO: 371.

In some embodiments, the antibody or antigen-binding fragment that binds to human TMPRSS4 is or is derived from TMPRSS4 Ab59. In some embodiments, the antibody or antigen-binding fragment that binds to TMPRSS4 comprises a VH comprising an HCDR1 comprising the amino acid sequence set forth in SEQ ID NO: 378, an HCDR2 comprising the amino acid sequence set forth in SEQ ID NO: 379, and an HCDR3 comprising the amino acid sequence set forth in SEQ ID NO: 380, and a VL comprising an LCDR1 comprising the amino acid sequence set forth in SEQ ID NO: 175, an LCDR2 comprising the amino acid sequence set forth in SEQ ID NO: 38, and an LCDR3 comprising the amino acid sequence set forth in SEQ ID NO: 381 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 376 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 377. In some embodiments, to TMPRSS4 comprises a VH comprising the amino acid sequence set forth in SEQ ID NO: 376 and a VL comprising the amino acid sequence set forth in SEQ ID NO: 377.

In some embodiments, the antibody or antigen-binding fragment that binds to human TMPRSS4 is or is derived from TMPRSS4 Ab60. In some embodiments, the antibody or antigen-binding fragment that binds to TMPRSS4 comprises a VH comprising an HCDR1 comprising the amino acid sequence set forth in SEQ ID NO: 384, an HCDR2 comprising the amino acid sequence set forth in SEQ ID NO: 58, and an HCDR3 comprising the amino acid sequence set forth in SEQ ID NO: 385, and a VL comprising an LCDR1 comprising the amino acid sequence set forth in SEQ ID NO: 386, an LCDR2 comprising the amino acid sequence set forth in SEQ ID NO: 38, and an LCDR3 comprising the amino acid sequence set forth in SEQ ID NO: 387 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 382 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 383. In some embodiments, to TMPRSS4 comprises a VH comprising the amino acid sequence set forth in SEQ ID NO: 382 and a VL comprising the amino acid sequence set forth in SEQ ID NO: 383.

In some embodiments, the antibody or antigen-binding fragment that binds to human TMPRSS4 is or is derived from TMPRSS4 Ab61. In some embodiments, the antibody or antigen-binding fragment that binds to TMPRSS4 comprises a VH comprising an HCDR1 comprising the amino acid sequence set forth in SEQ ID NO: 93, an HCDR2 comprising the amino acid sequence set forth in SEQ ID NO: 390, and an HCDR3 comprising the amino acid sequence set forth in SEQ ID NO: 163, and a VL comprising an LCDR1 comprising the amino acid sequence set forth in SEQ ID NO: 75, an LCDR2 comprising the amino acid sequence set forth in SEQ ID NO: 38, and an LCDR3 comprising the amino acid sequence set forth in SEQ ID NO: 391 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 388 and a VL comprising an amino acid sequence at least 80%, at least 85%, at least 90%, at least 98%, at least 99%, at least 99.5% or 100% identical to the amino acid sequence set forth in SEQ ID NO: 389. In some embodiments, to TMPRSS4 comprises a VH comprising the amino acid sequence set forth in SEQ ID NO: 388 and a VL comprising the amino acid sequence set forth in SEQ ID NO: 389.

In some embodiments, the antibody or antigen-binding fragment that binds to human TMPRSS4 is or is derived from TMPRSS4 Ab62. In some embodiments, the antibody or antigen-binding fragment that binds to TMPRSS4 comprises a VH comprising an HCDR1 comprising the amino acid sequence set forth in SEQ ID NO: 394, an HCDR2 comprising the amino acid sequence set forth in SEQ ID NO: 395, and an HCDR3 comprising the amino acid sequence set forth in SEQ ID NO: 396, and a VL comprising an LCDR1 comprising the amino acid sequence set forth in SEQ ID NO: 44, an LCDR2 comprising the amino acid sequence set forth in SEQ ID NO: 45, and an LCDR3 comprising the amino acid sequence set forth in SEQ ID NO: 397 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 392 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 393. In some embodiments, to TMPRSS4 comprises a VH comprising the amino acid sequence set forth in SEQ ID NO: 392 and a VL comprising the amino acid sequence set forth in SEQ ID NO: 393.

In some embodiments, the antibody or antigen-binding fragment that binds to human TMPRSS4 is or is derived from TMPRSS4 Ab63. In some embodiments, the antibody or antigen-binding fragment that binds to TMPRSS4 comprises a VH comprising an HCDR1 comprising the amino acid sequence set forth in SEQ ID NO: 400, an HCDR2 comprising the amino acid sequence set forth in SEQ ID NO: 401, and an HCDR3 comprising the amino acid sequence set forth in SEQ ID NO: 402, and a VL comprising an LCDR1 comprising the amino acid sequence set forth in SEQ ID NO: 44, an LCDR2 comprising the amino acid sequence set forth in SEQ ID NO: 45, and an LCDR3 comprising the amino acid sequence set forth in SEQ ID NO: 403 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 398 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 399. In some embodiments, to TMPRSS4 comprises a VH comprising the amino acid sequence set forth in SEQ ID NO: 398 and a VL comprising the amino acid sequence set forth in SEQ ID NO: 399.

In some embodiments, the antibody or antigen-binding fragment that binds to human TMPRSS4 is or is derived from TMPRSS4 Ab64. In some embodiments, the antibody or antigen-binding fragment that binds to TMPRSS4 comprises a VH comprising an HCDR1 comprising the amino acid sequence set forth in SEQ ID NO: 193, an HCDR2 comprising the amino acid sequence set forth in SEQ ID NO: 406, and an HCDR3 comprising the amino acid sequence set forth in SEQ ID NO: 407, and a VL comprising an LCDR1 comprising the amino acid sequence set forth in SEQ ID NO: 44, an LCDR2 comprising the amino acid sequence set forth in SEQ ID NO: 45, and an LCDR3 comprising the amino acid sequence set forth in SEQ ID NO: 408 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 404 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 405. In some embodiments, to TMPRSS4 comprises a VH comprising the amino acid sequence set forth in SEQ ID NO: 404 and a VL comprising the amino acid sequence set forth in SEQ ID NO: 405.

In some embodiments, the antibody or antigen-binding fragment that binds to human TMPRSS4 is or is derived from TMPRSS4 Ab65. In some embodiments, the antibody or antigen-binding fragment that binds to TMPRSS4 comprises a VH comprising an HCDR1 comprising the amino acid sequence set forth in SEQ ID NO: 193, an HCDR2 comprising the amino acid sequence set forth in SEQ ID NO: 194, and an HCDR3 comprising the amino acid sequence set forth in SEQ ID NO: 411, and a VL comprising an LCDR1 comprising the amino acid sequence set forth in SEQ ID NO: 44, an LCDR2 comprising the amino acid sequence set forth in SEQ ID NO: 412, and an LCDR3 comprising the amino acid sequence set forth in SEQ ID NO: 413 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 409 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 410. In some embodiments, to TMPRSS4 comprises a VH comprising the amino acid sequence set forth in SEQ ID NO: 409 and a VL comprising the amino acid sequence set forth in SEQ ID NO: 410.

In some embodiments, the antibody or antigen-binding fragment that binds to human TMPRSS4 is or is derived from TMPRSS4 Ab66. In some embodiments, the antibody or antigen-binding fragment that binds to TMPRSS4 comprises a VH comprising an HCDR1 comprising the amino acid sequence set forth in SEQ ID NO: 220, an HCDR2 comprising the amino acid sequence set forth in SEQ ID NO: 416, and an HCDR3 comprising the amino acid sequence set forth in SEQ ID NO: 417, and a VL comprising an LCDR1 comprising the amino acid sequence set forth in SEQ ID NO: 418, an LCDR2 comprising the amino acid sequence set forth in SEQ ID NO: 38, and an LCDR3 comprising the amino acid sequence set forth in SEQ ID NO: 419 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 414 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 415. In some embodiments, to TMPRSS4 comprises a VH comprising the amino acid sequence set forth in SEQ ID NO: 414 and a VL comprising the amino acid sequence set forth in SEQ ID NO: 415.

In some embodiments, the antibody or antigen-binding fragment that binds to human TMPRSS4 is or is derived from TMPRSS4 Ab67. In some embodiments, the antibody or antigen-binding fragment that binds to TMPRSS4 comprises a VH comprising an HCDR1 comprising the amino acid sequence set forth in SEQ ID NO: 19, an HCDR2 comprising the amino acid sequence set forth in SEQ ID NO: 4, and an HCDR3 comprising the amino acid sequence set forth in SEQ ID NO: 422, and a VL comprising an LCDR1 comprising the amino acid sequence set forth in SEQ ID NO: 44, an LCDR2 comprising the amino acid sequence set forth in SEQ ID NO: 45, and an LCDR3 comprising the amino acid sequence set forth in SEQ ID NO: 423 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 420 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 421. In some embodiments, to TMPRSS4 comprises a VH comprising the amino acid sequence set forth in SEQ ID NO: 420 and a VL comprising the amino acid sequence set forth in SEQ ID NO: 421.

In some embodiments, the antibody or antigen-binding fragment that binds to human TMPRSS4 is or is derived from TMPRSS4 Ab68. In some embodiments, the antibody or antigen-binding fragment that binds to TMPRSS4 comprises a VH comprising an HCDR1 comprising the amino acid sequence set forth in SEQ ID NO: 426, an HCDR2 comprising the amino acid sequence set forth in SEQ ID NO: 427, and an HCDR3 comprising the amino acid sequence set forth in SEQ ID NO: 428, and a VL comprising an LCDR1 comprising the amino acid sequence set forth in SEQ ID NO: 139, an LCDR2 comprising the amino acid sequence set forth in SEQ ID NO: 15, and an LCDR3 comprising the amino acid sequence set forth in SEQ ID NO: 429 and/or the antibody or antigen-binding fragment that binds to TMPRSS4 comprises a VH comprising an amino acid sequence at least 80%, at least 85%, at least 90%, at least 91%, least 99%, at least 99.5% or 100% identical to the amino acid sequence set forth in SEQ ID NO: 424 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 425. In some embodiments, to TMPRSS4 comprises a VH comprising the amino acid sequence set forth in SEQ ID NO: 424 and a VL comprising the amino acid sequence set forth in SEQ ID NO: 425.

In some embodiments, the antibody or antigen-binding fragment that binds to human TMPRSS4 is or is derived from TMPRSS4 Ab69. In some embodiments, the antibody or antigen-binding fragment that binds to TMPRSS4 comprises a VH comprising an HCDR1 comprising the amino acid sequence set forth in SEQ ID NO: 432, an HCDR2 comprising the amino acid sequence set forth in SEQ ID NO: 433, and an HCDR3 comprising the amino acid sequence set forth in SEQ ID NO: 434, and a VL comprising an LCDR1 comprising the amino acid sequence set forth in SEQ ID NO: 238, an LCDR2 comprising the amino acid sequence set forth in SEQ ID NO: 435, and an LCDR3 comprising the amino acid sequence set forth in SEQ ID NO: 436 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 430 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 431. In some embodiments, to TMPRSS4 comprises a VH comprising the amino acid sequence set forth in SEQ ID NO: 430 and a VL comprising the amino acid sequence set forth in SEQ ID NO: 431.

In some embodiments, the antibody or antigen-binding fragment that binds to human TMPRSS4 is or is derived from TMPRSS4 Ab70. In some embodiments, the antibody or antigen-binding fragment that binds to TMPRSS4 comprises a VH comprising an HCDR1 comprising the amino acid sequence set forth in SEQ ID NO: 439, an HCDR2 comprising the amino acid sequence set forth in SEQ ID NO: 440, and an HCDR3 comprising the amino acid sequence set forth in SEQ ID NO: 441, and a VL comprising an LCDR1 comprising the amino acid sequence set forth in SEQ ID NO: 442, an LCDR2 comprising the amino acid sequence set forth in SEQ ID NO: 443, and an LCDR3 comprising the amino acid sequence set forth in SEQ ID NO: 444 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 437 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 438. In some embodiments, to TMPRSS4 comprises a VH comprising the amino acid sequence set forth in SEQ ID NO: 437 and a VL comprising the amino acid sequence set forth in SEQ ID NO: 438.

In some embodiments, the antibody or antigen-binding fragment that binds to human TMPRSS4 is or is derived from TMPRSS4 Ab71. In some embodiments, the antibody or antigen-binding fragment that binds to TMPRSS4 comprises a VH comprising an HCDR1 comprising the amino acid sequence set forth in SEQ ID NO: 447, an HCDR2 comprising the amino acid sequence set forth in SEQ ID NO: 448, and an HCDR3 comprising the amino acid sequence set forth in SEQ ID NO: 449, and a VL comprising an LCDR1 comprising the amino acid sequence set forth in SEQ ID NO: 450, an LCDR2 comprising the amino acid sequence set forth in SEQ ID NO: 15, and an LCDR3 comprising the amino acid sequence set forth in SEQ ID NO: 451 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 445 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 446. In some embodiments, to TMPRSS4 comprises a VH comprising the amino acid sequence set forth in SEQ ID NO: 445 and a VL comprising the amino acid sequence set forth in SEQ ID NO: 446.

In some embodiments, the antibody or antigen-binding fragment that binds to human TMPRSS4 is or is derived from TMPRSS4 Ab72. In some embodiments, the antibody or antigen-binding fragment that binds to TMPRSS4 comprises a VH comprising an HCDR1 comprising the amino acid sequence set forth in SEQ ID NO: 454, an HCDR2 comprising the amino acid sequence set forth in SEQ ID NO: 455, and an HCDR3 comprising the amino acid sequence set forth in SEQ ID NO: 456, and a VL comprising an LCDR1 comprising the amino acid sequence set forth in SEQ ID NO: 457, an LCDR2 comprising the amino acid sequence set forth in SEQ ID NO: 15, and an LCDR3 comprising the amino acid sequence set forth in SEQ ID NO: 112 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 452 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 453. In some embodiments, to TMPRSS4 comprises a VH comprising the amino acid sequence set forth in SEQ ID NO: 452 and a VL comprising the amino acid sequence set forth in SEQ ID NO: 453.

In some embodiments, the antibody or antigen-binding fragment that binds to human TMPRSS4 is or is derived from TMPRSS4 Ab73. In some embodiments, the antibody or antigen-binding fragment that binds to TMPRSS4 comprises a VH comprising an HCDR1 comprising the amino acid sequence set forth in SEQ ID NO: 180, an HCDR2 comprising the amino acid sequence set forth in SEQ ID NO: 460, and an HCDR3 comprising the amino acid sequence set forth in SEQ ID NO: 461, and a VL comprising an LCDR1 comprising the amino acid sequence set forth in SEQ ID NO: 462, an LCDR2 comprising the amino acid sequence set forth in SEQ ID NO: 463, and an LCDR3 comprising the amino acid sequence set forth in SEQ ID NO: 464 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 458 and a VL comprising an amino acid sequence at least 80%, at least 85%, at least 90%, at least 98%, at least 99%, at least 99.5% or 100% identical to the amino acid sequence set forth in SEQ ID NO: 459. In some embodiments, to TMPRSS4 comprises a VH comprising the amino acid sequence set forth in SEQ ID NO: 458 and a VL comprising the amino acid sequence set forth in SEQ ID NO: 459.

In some embodiments, the antibody or antigen-binding fragment that binds to human TMPRSS4 is or is derived from TMPRSS4 Ab74. In some embodiments, the antibody or antigen-binding fragment that binds to TMPRSS4 comprises a VH comprising an HCDR1 comprising the amino acid sequence set forth in SEQ ID NO: 180, an HCDR2 comprising the amino acid sequence set forth in SEQ ID NO: 467, and an HCDR3 comprising the amino acid sequence set forth in SEQ ID NO: 468, and a VL comprising an LCDR1 comprising the amino acid sequence set forth in SEQ ID NO: 469, an LCDR2 comprising the amino acid sequence set forth in SEQ ID NO: 470, and an LCDR3 comprising the amino acid sequence set forth in SEQ ID NO: 471 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 465 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 466. In some embodiments, to TMPRSS4 comprises a VH comprising the amino acid sequence set forth in SEQ ID NO: 465 and a VL comprising the amino acid sequence set forth in SEQ ID NO: 466.

In some embodiments, the antibody or antigen-binding fragment that binds to human TMPRSS4 is or is derived from TMPRSS4 Ab75. In some embodiments, the antibody or antigen-binding fragment that binds to TMPRSS4 comprises a VH comprising an HCDR1 comprising the amino acid sequence set forth in SEQ ID NO: 474, an HCDR2 comprising the amino acid sequence set forth in SEQ ID NO: 475, and an HCDR3 comprising the amino acid sequence set forth in SEQ ID NO: 476, and a VL comprising an LCDR1 comprising the amino acid sequence set forth in SEQ ID NO: 477, an LCDR2 comprising the amino acid sequence set forth in SEQ ID NO: 478, and an LCDR3 comprising the amino acid sequence set forth in SEQ ID NO: 479 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 472 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 473. In some embodiments, to TMPRSS4 comprises a VH comprising the amino acid sequence set forth in SEQ ID NO: 472 and a VL comprising the amino acid sequence set forth in SEQ ID NO: 473.

In some embodiments, the antibody or antigen-binding fragment that binds to human TMPRSS4 is or is derived from TMPRSS4 Ab76. In some embodiments, the antibody or antigen-binding fragment that binds to TMPRSS4 comprises a VH comprising an HCDR1 comprising the amino acid sequence set forth in SEQ ID NO: 474, an HCDR2 comprising the amino acid sequence set forth in SEQ ID NO: 482, and an HCDR3 comprising the amino acid sequence set forth in SEQ ID NO: 483, and a VL comprising an LCDR1 comprising the amino acid sequence set forth in SEQ ID NO: 44, an LCDR2 comprising the amino acid sequence set forth in SEQ ID NO: 484, and an LCDR3 comprising the amino acid sequence set forth in SEQ ID NO: 485 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 480 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 481. In some embodiments, to TMPRSS4 comprises a VH comprising the amino acid sequence set forth in SEQ ID NO: 480 and a VL comprising the amino acid sequence set forth in SEQ ID NO: 481.

In some embodiments, the antibody or antigen-binding fragment that binds to human TMPRSS4 is or is derived from TMPRSS4 Ab77. In some embodiments, the antibody or antigen-binding fragment that binds to TMPRSS4 comprises a VH comprising an HCDR1 comprising the amino acid sequence set forth in SEQ ID NO: 488, an HCDR2 comprising the amino acid sequence set forth in SEQ ID NO: 489, and an HCDR3 comprising the amino acid sequence set forth in SEQ ID NO: 490, and a VL comprising an LCDR1 comprising the amino acid sequence set forth in SEQ ID NO: 44, an LCDR2 comprising the amino acid sequence set forth in SEQ ID NO: 45, and an LCDR3 comprising the amino acid sequence set forth in SEQ ID NO: 491 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 486 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 487. In some embodiments, to TMPRSS4 comprises a VH comprising the amino acid sequence set forth in SEQ ID NO: 486 and a VL comprising the amino acid sequence set forth in SEQ ID NO: 487.

In some embodiments, the antibody or antigen-binding fragment that binds to human TMPRSS4 is or is derived from TMPRSS4 Ab78. In some embodiments, the antibody or antigen-binding fragment that binds to TMPRSS4 comprises a VH comprising an HCDR1 comprising the amino acid sequence set forth in SEQ ID NO: 494, an HCDR2 comprising the amino acid sequence set forth in SEQ ID NO: 495, and an HCDR3 comprising the amino acid sequence set forth in SEQ ID NO: 496, and a VL comprising an LCDR1 comprising the amino acid sequence set forth in SEQ ID NO: 497, an LCDR2 comprising the amino acid sequence set forth in SEQ ID NO: 15, and an LCDR3 comprising the amino acid sequence set forth in SEQ ID NO: 498 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 492 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 493. In some embodiments, to TMPRSS4 comprises a VH comprising the amino acid sequence set forth in SEQ ID NO: 492 and a VL comprising the amino acid sequence set forth in SEQ ID NO: 493.

In some embodiments, the antibody or antigen-binding fragment that binds to human TMPRSS4 is or is derived from TMPRSS4 Ab79. In some embodiments, the antibody or antigen-binding fragment that binds to TMPRSS4 comprises a VH comprising an HCDR1 comprising the amino acid sequence set forth in SEQ ID NO: 501, an HCDR2 comprising the amino acid sequence set forth in SEQ ID NO: 502, and an HCDR3 comprising the amino acid sequence set forth in SEQ ID NO: 503, and a VL comprising an LCDR1 comprising the amino acid sequence set forth in SEQ ID NO: 324, an LCDR2 comprising the amino acid sequence set forth in SEQ ID NO: 15, and an LCDR3 comprising the amino acid sequence set forth in SEQ ID NO: 252 and/or the antibody or antigen-binding fragment that binds to TMPRSS4 comprises a VH comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 499 and a VL comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 500. In some embodiments, to TMPRSS4 comprises a VH comprising the amino acid sequence set forth in SEQ ID NO: 499 and a VL comprising the amino acid sequence set forth in SEQ ID NO: 500.

In various embodiments, the antigen-binding fragment that binds to TMPRSS4 comprises an scFv. In some embodiments, the scFv has the format VH-L-VL or VL-L-VH, wherein L is a linker peptide and the VH and VL are any VH and VL disclosed herein. In some embodiments, the scFv has the format VH-L-VL, wherein L is a linker peptide. In some embodiments, the scFv has the format VL-L-VH, wherein L is a linker peptide. In some embodiments, the linker peptide comprises the amino acid sequence of GGGGSGGGGSGGGGS (SEQ ID NO: 819). In some embodiments, the linker peptide comprises the amino acid sequence of GGGGSGSGGGGSGGGGS (SEQ ID NO: 820). Table 5 provides exemplary amino acid sequences of scFvs that bind to TMPRSS4. The linker peptide linking the VH to the VL is indicated in bold italic text.

TABLE 5
TMPRSS4 scFv Amino Acid Sequences

| Clone | scFv Sequence |
|---|---|
| TMPRSS4 | QVQLVQSGAEVKKPGSSVKVSCKASGYTFTDYYMHWVRQAPGQGLEWMGGIIPIFGTANYAQKFQGR |
| Ab1 scFv | VTITADESTSTAYMELSSLRSEDTAVYYCAKEGANGYWGQGTLVTVSSGGGGSGGGGSGGGGSDIQM |
| (VH-VL) | TQSPSSLSASVGDRVTITCQASQDISNYLNWYQQKPGKAPKLLIYKASSLESGVPSRFSGSGSGTDF |
| | TLTISSLQPEDFATYYCQQSSRIPPTFGQGTKVEIK (SEQ ID NO: 504) |
| TMPRSS4 | EVQLLESGGGLVQPGGSLRLSCAASGFTFSDYYMSWVRQAPGKGLEWVSYISGSGDAIYYADSVKGR |
| Ab2 scFv | FTISRDNSKNTLYLQMNSLRAEDTAVYYCARDRSDCGGDDRFLCDGYFDLWGRGTLVSLSGGGGSGG |
| (VH-VL) | GGSGGGGSDIQMTQSPSSLSASVGDRVTITCRASQSTNNYVNWYQQKPGKAPKLLIYAASSLQSGVP |
| | SRFSGSGSGTDFTLTISSLQPEDFATYYCQQSYSIPLTFGPGTKVDIK (SEQ ID NO: 505) |
| TMPRSS4 | QVQLVQSGAEVKKPGSSVKVSCKASGYTFTSYDINWVRQAPGQGLEWMGGIIPIFGTTKFAQKFQGR |
| Ab3 scFv | VTITADESTSTAYMELSSLRSEDTAVYYCARDWYSSSWYNGDRGDWFDPWGQGTLVTVSSGGGGSGG |
| (VH-VL) | GGSGGGGSDIVMTQSPLSLPVTPGEPASISCRSSGSLLHSNGYNYLDWYLQKPGQSPQLLIYAASSL |
| | QSGVPDRFSGSGSGTDFTLKISRVEAEDVGVYYCMQGTHWPGTFGQGTKVEIK (SEQ ID NO: 506) |
| TMPRSS4 | QVQLVQSGAEVKKPGASVKVSCKASGYFFTTYYLHWVRQAPGQGLEWMGVINPNSRLTSYAESFQGR |
| Ab4 scFv | VTMTRDTSTSTVYMELSSLRSEDTAVYYCEREMFPSSYGIDVWGQGTTVTVSSGGGGSGSGGGGSGG |
| (VH-VL) | GGSDIVMTQSPLSLPVTPGEPASISCRSSQSLLHSNGYNYLDWYLQKPGQSPQLLIYAASTLQSGVP |
| | DRFSGSGSGTDFTLKISRVEAEDVGVYYCMQGTHWPPTFGQGTKLEIK (SEQ ID NO: 507) |
| TMPRSS4 | QVQLVQSGAEVKKPGSSVKVSCKASGYTFTSYYMHWVRQAPGQGLEWMGRIIPILGATDYAQKFQGR |
| Ab5 scFv | VTITADESTSTAYMELSSLRSEDTAVYYCARAGYSSIAARPAFWGQGTLVTVSSGGGGSGSGGGGSG |
| (VH-VL) | GGGSEIVMTQSPATLSVSPGERATLSCRASQSVSSNLAWYQQKPGQAPRLLIYGASTRATGIPARFS |
| | GSGSGTEFTLTISSLQSEDFAVYYCQQYYSPFPLTFGGGTKVEIK (SEQ ID NO: 508) |
| TMPRSS4 | QVQLVQSGAEVKKPGASVKVSCKASGYTFTSYYMHWVRQAPGQGLEWLGIINPSDYTTSYAQKFQGR |
| Ab6 scFv | VTMTRDTSTSTVYMELSSLRSEDTAVYYCARVASSSWYPGDENWYFDLWGRGTLVTVSSGGGGSGSG |
| (VH-VL) | GGGSGGGGSDIVMTQSPDSLAVSLGERATINCKSSQSVLYSSNNKNYLAWYQQKPGQPPKLLIYWAS |
| | TRESGVPDRFSGSGSGTDFTLTISSLQAEDVAIYYCQQYYAIPWTFGQGTKVEIK (SEQ ID NO: |
| | 509) |
| TMPRSS4 | QVQLVQSGAEVKKPGASVKVSCKASGYTFSRYFMHWVRQAPGQGLEWVGWINPNSGNTGYAQKFQGR |
| Ab7 scFv | VTMTRDTSTSTVYMELSSLRSEDTAVYYCARVVTGGRLDVWGQGTTVTVSSGGGGSGGGGSGGGGSE |
| (VH-VL) | IVMTQSPATLSVSPGERATLSCRASQRVSNNYLAWYQQKPGQAPRLLIYGASTRASGIPARFSGSGS |
| | GTEFTLTISSLQSEDFAVYYCQQYGSTPYTFGQGTKVEIK (SEQ ID NO: 510) |
| TMPRSS4 | QVQLVQSGAEVKKPGASVKVSCKASGYRFTSQYMHWVRQAPGQGLEWMGIINPSGGSTSYAQKFQGR |
| Ab8 scFv | VTMTRDTSTSTVYMELSSLRSEDTAVYYCARGRIAVAGHPLGYWGQGTLVTVSSGGGGSGGGGSGGG |
| (VH-VL) | GSDIQMTQSPSSLSASVGDRVTITCRASQSISSWLAWYQQKPGKAPKLLIYGASSLQSGVPSRFSGS |
| | GSGTDFTLTISSLQPEDFATYYCQQANSFPPTFGGGTKVEIK (SEQ ID NO: 511) |
| TMPRSS4 | QVQLVQSGAEVKKPGASVKVSCKASGYTFTRYYMHWVRQAPGQGLEWMGWINPNSGGTNYAQKFQGR |
| Ab9 | VTMTRDTSTSTVYMELSSLRSEDTAVYYCARGGSWGSGPLGYWGQGTLVTVSSGGGGSGGGGSGGGG |
| | SDIQMTQSPSSLSASVGDRVTITCQASQDISRFLHWYQQKPGKAPKLLIYGASNLKSGVPSRFSGSG |
| | SGTDFTLTISSLQPEDFATYYCQQSYSTPPTFGGGTKVEIK (SEQ ID NO: 512) |
| TMPRSS4 | QVQLVQSGAEVKKPGASVKVSCKASGYTFTSYYMHWVRQAPGQGLEWMGIINPSGAGTTYGHNFQGR |
| Ab10 | VTMTRDTSTSTVYMELSSLRSEDTAVYYCARGPRDTAMVRFDYWGQGTLVTVSSGGGGSGGGGSGGG |
| | GSEIVMTQSPATLSVSPGERATLSCRASQSVSSYLAWYQQKPGQAPRLLIYGASTRATGIPARFSGS |
| | GSGTEFTLTISSLQSEDFAVYYCQQYGSSPGTFGQGTKLEIK (SEQ ID NO: 513) |
| TMPRSS4 | QVQLVQSGAEVKKPGASVKVSCKASGGTFSNYAISWVRQAPGQGLEWVGRINPNSGGTNYAQKFQGR |
| Ab11 scFv | VTMTRDTSTSTVYMELSSLRSEDTAVYYCARGRYSSSSWGQGTLVTVSSGGGGSGGGGSGGGGSDIQ |
| (VH-VL) | MTQSPSSLSASVGDRVTITCRASQSINNYLNWYQQKPGKAPKLLIYAASSLQSGVPSRFSGSGSGTD |
| | FTLTISSLQPEDFATYYCQQSYSTKWTFGQGTKVEIK (SEQ ID NO: 514) |
| TMPRSS4 | QVQLVQSGAEVKKPGASVKVSCKASGYTFSNYYIHWVRQAPGQGLEWMGWINPNSGDTNYAQKFQGR |
| Ab12 scFv | VTMTRDTSTSTVYMELSSLRSEDTAVYYCARGMTWRTSAATYWGQGTLVTVSSGGGGSGGGGSGGGG |
| (VH-VL) | SDIQMTQSPSSLSASVGDRVTITCRASQGISRWLAWYQQKPGKAPKLLIYGASNLQTGVPSRFSGSG |
| | SGTDFTLTISSLQPEDFATYYCQQSYSTPPTFGPGTKVDIK (SEQ ID NO: 515) |
| TMPRSS4 | QVQLVQSGAEVKKPGASVKVSCKASGYTFTNYYMHWVRQAPGQGLEWMGWISAYNGNTNYAQKLQGR |
| Ab13 scFv | VTMTRDTSTSTVYMELSSLRSEDTAVYYCATASGWGHSNSAGYWGQGTLVTVSSGGGGSGGGGSGGG |
| (VH-VL) | GSEIVMTQSPATLSVSPGERATLSCRASQSVNGNYLAWYQQKPGQAPRLLIYGVSSRASGIPARFSG |
| | SGSGTEFTLTISSLQSEDFAVYYCQQYGSSPYTFGQGTKVEIK (SEQ ID NO: 516) |
| TMPRSS4 | EVQLLESGGGLVQPGGSLRLSCAASGFTFSNHYMSWVRQAPGKGLEWVSAISGSGGSTYYADSVKGR |
| Ab14 scFv | FTISRDNSKNTLYLQMNSLRAGDTAVYYCARDRYRWGRGYFQHWGQGTLVTVSSGGGGSGGGGSGGG |
| (VH-VL) | GSDIQMTQSPSSLSASVGDRVTITCRASQRISTYLNWYQQKPGKAPKLLIYSASTLQAGVPSRFSGS |
| | GSGTDFTLTISSLQPEDFATYYCQQAYSLPWTFGQGTKLEIK (SEQ ID NO: 517) |
| TMPRSS4 | QVQLVQSGAEVKKPGASVKVSCKASGYTFTRYYMHWVRQAPGQGLEWMGWINPNSGVTNFAQKFQGR |
| Ab15 scFv | VTMTRDTSTSTVYMELSSLRSEDTAVYYCARVRIGWLQSPPLYWGQGTLVTVSSGGGGSGGGGSGGG |
| (VH-VL) | GSDIQMTQSPSSLSASVGDRVTITCRASQSINTWLAWYQQKPGKAPKLLIYAASSLQSGVPSRFSGS |
| | GSGTDFTLTISSLQPEDFATYYCQQAISFPLTFGGGTKVEIK (SEQ ID NO: 518) |
| TMPRSS4 | QVQLVQSGAEVKKPGASVKVSCKASGYTFSRHYMHWVRQAPGQGLEWMGRINPNSGGTNYAQKFQGR |
| Ab16 scFv | VTMTRDTSTSTVYMELSSLRSEDTAVYYCARSIYGDYWFDPWGQGTLVTVSSGGGGSGGGGSGGGGS |
| (VH-VL) | DIQMTQSPSSLSASVGDRVTITCRASQSINRWLAWYQQKPGKAPKLLIYGASNLQSGVPSRFSGSGS |
| | GTDFTLTISSLQPEDFATYYCQQANSFPYTFGQGTKLEIK (SEQ ID NO: 519) |
| TMPRSS4 | QVQLVQSGAEVKKPGASVKVSCKASGYTFTSYYIHWVRQAPGQGLEWMGWMSPNSGDTGYAQKFQGR |
| Ab17 scFv | VTMTRDTSTSTVYMELSSLRSEDTAVYYCARLVRGGFDYWGQGTLVTVSSGGGGSGGGGSGGGGSDI |
| (VH-VL) | QMTQSPSSLSASVGDRVTITCRASQGISSYLNWYQQKPGKAPKLLIYAASRLQSGVPSRFSGSGSGT |
| | DFTLTISSLQPEDFATYYCQQSYRSPPTFGQGTKLEIK (SEQ ID NO: 520) |
| TMPRSS4 | QVQLVQSGAEVKKPGASVKVSCKASGYTFTSSGINWVRQAPGQGLEWMGWINPNSGGAKYAQRFQGR |
| Ab18 scFv | VTMTRDTSTSTVYMELSSLRSEDTAVYYCARARGYSGSKRDFQHWGQGTLVTVSSGGGGSGGGGSGG |
| (VH-VL) | GGSDIVMTQSPDSLAVSLGERATINCKSSQSVLYSSNNKNYLAWYQQKPGQPPKLLIYWASTRESGV |
| | PDRFSGSGSGTDFTLTISSLQAEDVAVYHCQQYYNTPFTFGPGTKVDIK (SEQ ID NO: 521) |
| TMPRSS4 | QVQLVQSGAEVKKPGASVKVSCKASGYTFNRKFMHWVRQAPGQGLEWMGWMNPNNGATNYAQKFQGR |
| Ab19 scFv | VTMTRDTSTSTVYMELSSLRSEDTAVYYCARGRGYYGSGSYYGDYWGQGTLVTVSSGGGGSGGGGSG |
| (VH-VL) | GGGSDIQMTQSPSSLSASVGDRVTITCRASQSISRYLNWYQQKPGKAPKLLIYGASNLQSGVPSRFS |
| | GSGSGTDFTLTISSLQPEDFATYYCQQSYSTPPTFGPGTKVDIK (SEQ ID NO: 522) |
| TMPRSS4 | QVQLVQSGAEVKKPGASVKVSCKASGYTFTRYYMHWVRQAPGQGLEWMGWMNPNSGNAGYAQKLQGR |
| Ab20 scFv | VTMTRDTSTSTVYMELSSLRSEDTAVYYCARGYNWFDPWGQGTLVTVSSGGGGSGGGGSGGGGSDIQ |
| (VH-VL) | MTQSPSSLSASVGDRVTITCRASQNIATYLSWYQQKPGKAPKLLIYGASALRSGVPSRFSGSGSGTD |
| | FTLTISSLQPEDFATYYCLQHNTYPLTFGGGTKVEIK (SEQ ID NO: 523) |
| TMPRSS4 | QVQLVQSGAEVKKPGASVKVSCKASGYSFSGYYLHWVRQAPGQGLEWMGWMNPDSGNTGYAQNFQGR |
| Ab21 scFv | VTMTRDTSTSTVYMELSSLRSEDTAVYYCARLHRGGHDYWGQGTLVTVSSGGGGSGGGGSGGGGSDI |
| (VH-VL) | QMTQSPSSLSASVGDRVTITCRASQSISRYLNWYQQKPGKAPKLLIYAASTLQSGVPSRFSGSGSGT |
| | DFTLTISSLQPEDFATYYCQQSYSTPVTFGQGTRLEIK (SEQ ID NO: 524) |
| TMPRSS4 | QVQLVQSGAEVKKPGASVKVSCKASGYTFTSYYMHWVRQAPGQGLEWMGRINPHSGDADFVDKFQGR |
| Ab22 scFv | VTMTRDTSTSTVYMELSSLRSEDTAVYYCARDRRGYGGNSLDYWGQGTLVTVSSGGGGSGGGGSGGG |
| (VH-VL) | GSDIQMTQSPSSLSASVGDRVTITCRAGQNIKRYLNWYQQKPGKAPKLLIYAASSLQSGVPSRFSGS |
| | GSGTDFTLTISSLQPEDFATYYCQQSYSSPLTFGGGTKVEIK (SEQ ID NO: 525) |
| TMPRSS4 | QVQLVQSGAEVKKPGASVKVSCKASGYTFTRNYLHWVRQAPGQGLEWMGIINPSGGSTTYAQKFQGR |
| Ab23 scFv | VTMTRDTSTSTVYMELSSLRSEDTAVYYCARGRTWFRSGMDVWGQGTTVTVSSGGGGSGGGGSGGGG |
| (VH-VL) | SEIVMTQSPATLSVSPGERATLSCRASQSVGNYLAWYQQKPGQAPRLLIYGASTRATGIPARFSGSG |
| | SGTEFTLTISSLQSEDFAVYYCQQYHSSPPYTFGQGTKVEIK (SEQ ID NO: 526) |
| TMPRSS4 | QVQLVQSGAEVKKSGASVKVSCKASGYTFTSYYMHWVRQAPGQGLEWMGVINPSGGTTSYAQKFQGR |
| Ab24 scFv | VTMTRETSTSTVYMELSSLRSEDTAVYYCARGRGWLRSALGYWGQGTLVTVSSGGGGSGGGGSGGGG |
| (VH-VL) | SDIQMTQSPSSLSASVGDRVTITCRASQSISSWLAWYQQKPGKAPKLLIYAASTLQSGVPSRFSGSG |
| | SGTDFTLTISSLQPEDFATYYCQQSYNTPYTFGQGTKLEIK (SEQ ID NO: 527) |
| TMPRSS4 | QVQLVQSGAEVKKPGASVKVSCKASGGRFSTYALSWVRQAPGQGLEWMGWINPNSGGTNYAQKFQGR |
| Ab25 scFv | VTMTRDTSTSTVYMELSSLRSEDTAVYYCAKSLWWSPSHYYYYGMDVWGQGTTVTVSSGGGGSGGGG |
| (VH-VL) | SGGGGSEIVMTQSPATLSVSPGERATLSCRASQSVSSNYLAWYQQKPGQAPRLLIYGISTRASGIPA |
| | RFSGSGSGTEFTLTISSLQSEDFAVYYCQQRSNWPPSITFGQGTRLEIK (SEQ ID NO: 528) |
| TMPRSS4 | EVQLLESGGGLVQPGGSLRLSCAASGFTFSSYAMHWVRQAPGKGLEWVAVIWYDGSSKYYADSVKGR |
| Ab26 scFv | FTISRDNSKNTLYLQMNSLRAEDTAVYYCARGEVRRGFQHWGQGTLVTVSSGGGGSGGGGSGGGGSD |
| (VH-VL) | IQMTQSPSSLSASVGDRVTITCRASQNVGSWLAWYQQKPGKAPKLLIYAASSLQSGVPSRFSGSGSG |
| | TDFTLTIRSLQPEDFATYYCQQSYSTPITFGQGTRLEIK (SEQ ID NO: 529) |
| TMPRSS4 | QVQLVQSGAEVKKPGASVKVSCKASGYTFSRYYMHWVRQAPGQGLEWMGWMNPNSGDTGYAQKFQGR |
| Ab27 scFv | VTMTRDTSTSTVYMELSSLRSEDTAVYYCAKGREWLRSPFDYWGQGTLVTVSSGGGGSGGGGSGGGG |
| (VH-VL) | SDIQMTQSPSSLSASVGDRVTITCRASQSISTWLAWYQQKPGKAPKLLIYAASSLQSGVPSRFSGSG |
| | SGTDFTLTISSLQPEDFATYYCQQLSSYPLTFGQGTKVEIK (SEQ ID NO: 530) |
| TMPRSS4 | QVQLVQSGAEVKKPGASVKVSCKASGYTFTGYYMHWVRQAPGQGLEWMGWMNPNSGNTGYAQKFQGR |
| Ab28 scFv | VTMTRDTSTSTVYMELSSLRSEDTAVYYCARLRAKGGGFDYWGQGTLVTVSSGGGGSGGGGSGGGGS |
| (VH-VL) | DIQMTQSPSSLSASVGDRVTITCRASQGIGNYLAWYQQKPGKAPKLLIYAASSLESGVPSRFSGSGS |
| | GTDFTLTISSLQPEDFATYYCQQGYRFPPTFGPGTKVDIK (SEQ ID NO: 531) |
| TMPRSS4 | QVQLVQSGAEVKKPGASVKVSCKASGYTFANYNIHWVRQAPGQGLEWMGWMNPNSGNTGYAQKFQGR |
| Ab29 scFv | VTMTRDTSTSTVYMELSSLRSEDTAVYYCARPRYSSGWYGWYFDLWGRGTLVTVSSGGGGSGGGGSG |
| (VH-VL) | GGGSDIVMTQSPLSLPVTPGEPASISCRSSQSLLHSNGYNYLDWYLQKPGQSPQLLIYLGSNRASGV |
| | PDRFSGSGSGTDFTLKISRVEAEDVGVYYCMQSTYWPPTFGQGTKLEIK (SEQ ID NO: 532) |
| TMPRSS4 | QVQLVQSGAEVKKPGASVKVSCKASGYTFTTYYMHWVRQAPGQGLEWMGWMNPNSGNTGYAQKFQGR |
| Ab30 scFv | VTMTRDTSTSTVYMELSSLRSEDTAVYYCARARTWLLSPFDYWGQGTLVTVSSGGGGSGGGGSGGGG |
| (VH-VL) | SEIVMTQSPATLSVSPGERATLSCRASQSVGRYLAWYQQKPGQAPRLLIYGASTRATGIPARFSGSG |
| | SGTEFTLTISSLQSEDFAVYYCQHYDSSPMYTFGQGTKLEIK (SEQ ID NO: 533) |
| TMPRSS4 | QVQLVQSGAEVKKPGSSVKVSCKASGYTFRGSGISWVRQAPGQGLEWMGIIYPADSETRYSPSFQGR |
| Ab31 scFv | VTITADESTSTAYMELSSLRSEDTAVYYCARESSSWDYFDYWGQGTLVTVSSGGGGSGGGGSGGGGS |
| (VH-VL) | EIVMTQSPATLSVSPGERATLSCRASQSVRSYLAWYQQKPGQAPRLLIYGASTRATGIPARFSGSGS |
| | GTEFTLTISSLQSEDFAVYYCQQHGSLPLTFGQGTKVEIK (SEQ ID NO: 534) |
| TMPRSS4 | QVQLVQSGAEVKKPGSSVKVSCKASGGTFSSYAISWVRQAPGQGLEWMGRINPSGGSTSYAQKFQGR |
| Ab32 scFv | VTMTRDTSTSTVYMELSSLRSEDTAVYYCARGRYSSSSWGQGTLVTVSSGGGGSGGGGSGGGGSDIQ |
| (VH-VL) | MTQSPSSLSASVGDRVTITCRASQSISTYLNWYQQKPGKAPKLLIYAASSLQRGVPSRFSGSGSGTD |
| | FTLTISSLQPEDFATYYCQQSYTTPLTFGGGTKVEIK (SEQ ID NO: 535) |
| TMPRSS4 | QVQLVQSGAEVKKPGASVKVSCKASGYTFSNYYMHWVRQAPGQGLEWVGWMNPKSGNTGYAQKFQGR |
| Ab33 scFv | VTMTRDTSTSTVYMELSSLRSEDTAVYYCARGRTWIQSSLGYWGQGTLVTVSSGGGGSGGGGSGGGG |
| (VH-VL) | SDIQMTQSPSSLSASVGDRVTITCRASQYISRWLAWYQQKPGKAPKLLIYGSSTLQSGVPSRFSGSG |
| | SGTDFTLTISSLQPEDFATYYCQQYYSTPFTFGPGTKLEIK (SEQ ID NO: 536) |
| TMPRSS4 | QVQLVQSGAEVKKPGASVKVSCKASGYTFTGYYIHWVRQAPGQGLEWMGWMNPHSGNTGYAQKFQGR |
| Ab34 scFv | VTMTRDTSTSTVYMELSSLRSEDTAVYYCAREGGRYSSGRLGYWGQGTLVTVSSGGGGSGGGGSGGG |
| (VH-VL) | GSDIQMTQSPSSLSASVGDRVTITCRASQGISSWLAWYQQKPGKAPKLLIYAASTLQTGVPSRFSGS |
| | GSGTDFTLTISSLQPEDFATYYCQQSKSIPITFGGGTKVEIK (SEQ ID NO: 537) |
| TMPRSS4 | QVQLVQSGAEVKKPGASVKVSCKASGYTFTGYYMHWVRQAPGQGLEWMGKISAHSGETKYAQNVQGR |
| Ab35 scFv | VTMTRDTSTSTVYMELSSLRSEDTAVYYCARANYYGDYVNYYYGMDVWGQGTTVTVSSGGGGSGGGG |
| (VH-VL) | SGGGGSDIQMTQSPSSLSASVGDRVTITCQASQDISNYLNWYQQKPGKAPKLLIYKASSLESGVPSR |
| | FSGSGSGTDFTLTISSLQPEDFATYYCQQTYTIPITFGQGTRLEIK (SEQ ID NO: 538) |
| TMPRSS4 | EVQLLESGGGLVQPGGSLRLSCAASGFTFSSRAMSWVRQAPGKGLEWVSRINYDGSATTYADSVKGR |
| Ab36 scFv | FTISRDNSKNTLYLQMNSLRAEDTAVYYCARGITIFGVFDYWGQGTLVTVSSGGGGSGGGGSGGGGS |
| (VH-VL) | DIQMTQSPSSLSASVGDRVTITCRASQSISTWLAWYQQKPGKAPKLLIYRASNLQSGVPSRFSGSGS |
| | GTDFTLTISSLQPEDFATYYCQQSYSTPLTFGGGTKVEIK (SEQ ID NO: 539) |
| TMPRSS4 | EVQLLESGGGLVQPGGSLRLSCAASGFTFSSYAMHWVRQAPGKGLEWVSYISSSGSTVYYADSVKGR |
| Ab37 scFv | FTISRDNSKNTLYLQMNSLRAEDTAVYYCARVSNVTPRSGFGYWGQGTLVTVSSGGGGSGGGGSGGG |
| (VH-VL) | GSDIQMTQSPSSLSASVGDRVTITCRASQSISRYLNWYQQKPGKAPKLLIYSASTLQSGVPSRFSGS |
| | GSGTDFTLTISSLQPEDFATYYCQQAHSFPPSFGQGTKLEIK (SEQ ID NO: 540) |
| TMPRSS4 | QVQLAQSGAEVKKPGASVKVSCKASGYTFTRHYIQWVRQAPGQGLEWMGWINPNSGNTGYAQKFQGR |
| Ab38 scFv | VTMTRDTSTSTVYMELSSLRSEDTAVYYCARGRQWLRGEYFQHWGQGTLVTVSSGGGGSGGGGSGGG |
| (VH-VL) | GSDIQMTQSPSSLSASVGDRVTITCQASQDISRYLNWYQQKPGKAPKLLIYGASNLLSGVPSRFSGS |
| | GSGTDFTLTISSLQPEDFATYYCQQTHTTPYTFGQGTRLEIK (SEQ ID NO: 541) |
| TMPRSS4 | QVQLVQSGAEVKKPGASVKVSCKASGGSFSGYAVSWVRQAPGQGLEWLGVINPSDSWTAFAQKFQGR |
| Ab39 scFv | VTMTRDTSTSTVYMELSSLRSEDTAVYYCAREREDDAFDIWGQGTTVTVSSGGGGSGGGGSGGGGSD |
| (VH-VL) | IQMTQSPSSLSASVGDRVTITCRASQGIRNWLAWYQQKPGKAPKLLIYRASTLQSGVPSRFSGSGSG |
| | TDFTLTISSLQPEDFATYYCQQSYTTPFTFGQGTKLEIK (SEQ ID NO: 542) |
| TMPRSS4 | QVQLVQSGAEVKKPGASVKVSCKASGGTFSSYAISWVRQAPGQGLEWMGIINPRGGSTNYAQKFQGR |
| Ab40 scFv | VTMTRDTSTSTVYMELSSLRSEDTAVYYCAREGSSWYYDAFDIWGQGTMVTVSSGGGGSGGGGSGGG |
| (VH-VL) | GSDIQMTQSPSSLSASVGDRVTITCRASQSISSYLNWYQQKPGKAPKLLIYAAYNLQSGVPSRFSGS |
| | GSGTDFTLTISSLQPEDFATYYCQQSYSIPFTFGGGTKVEIK (SEQ ID NO: 543) |
| TMPRSS4 | QVQLVQSGAEVKKPGASVKVSCKASGGTFSSYAISWVRQAPGQGLEWMGWMNPNSGDTHYAQKFQGR |
| Ab41 scFv | VTMTRDTSTSTVYMELSSLRSEDTAVYYCAREGSSWYYDAFDIWGQGTLVTVSSGGGGSGGGGSGGG |
| (VH-VL) | GSDIQMTQSPSSLSASVGDRVTITCRASQSISRWLAWYQQKPGKAPKLLIYAASTLQTGVPSRFSGS |
| | GSGTDFTLTISSLQPEDFATYYCLQHSSYPFTFGQGTKVEIK (SEQ ID NO: 544) |
| TMPRSS4 | QVQLVQSGAEVKKPGASVKVSCKASGYSFTSHYMHWVRQAPGQGLEWMGWMNPNSGNTGYAQKFQGR |
| Ab42 scFv | VTMTRDTSTSTVYMELSSLRSEDTAVYYCARLGQQLDYWGQGTLVTVSSGGGGSGGGGSGGGGSDIQ |
| (VH-VL) | MTQSPSSLSASVGDRVTITCRASQSIRNYLNWYQQKPGKAPKLLIYEASRLQSGVPSRFSGSGSGTD |
| | FTLTISSLQPEDFATYYCQQSYSAPPTFGPGTKVDIK (SEQ ID NO: 545) |
| TMPRSS4 | QVQLVQSGAEVKKPGASVKVSCKASGYTFIGYYMHWVRQAPGQGLEWMGRINPNSGETNYAQKFQGR |
| Ab43 scFv | VTMTRDTSTSTVYMELSSLRSEDTAVYYCARVRVRGVIHPGFDPWGQGTLVTVSSGGGGSGGGGSGG |
| (VH-VL) | GGSDIQMTQSPSSLSASVGDRVTITCQASQDISNYLNWYQQKPGKAPKLLIYAASSLQSGVPSRFSG |
| | SGSGTDFTLTISSLQPEDFATYYCQQSYSTPVTFGPGTKVDIK (SEQ ID NO: 546) |
| TMPRSS4 | QVQLVQSGAEVKKPGASVKVSCKASGYTFRNYYIHWVRQAPGQGLEWMGRINPNSGGTNYAQKFQGR |
| Ab44 scFv | VTMTRDTSTSTVYMELSSLRSEDTAVYYCARARIAVAVSGFGYWGQGTLVTVSSGGGGSGGGGSGGG |
| (VH-VL) | GSDIQMTQSPSSLSASVGDRVTITCQASQDISNYLNWYQQKPGKAPKLLIYAASSLHSGVPSRFSGS |
| | GSGTDFTLTISSLQPEDFATYYCQESSSFPYTFGPGTKVDIK (SEQ ID NO: 547) |
| TMPRSS4 | QVQLVQSGAEVKKPGASVKVSCKASGYTFSRWYMHWVRQAPGQGLEWMGRINPNSGGTNYAQKFQGR |
| Ab45 scFv | VTMTRDTSTSTVYMELSSLRSEDTAVYYCARVGGYGWFDPWGQGTLVTVSSGGGGSGGGGSGGGGSD |
| (VH-VL) | IQMTQSPSSLSASVGDRVTITCQATQDIRNYLNWYQQKPGKAPKLLIYATSSLQSGVPSRFSGSGSG |
| | TDFTLTISSLQPEDFATYYCQQSYSPPYTFGQGTKLEIK (SEQ ID NO: 548) |
| TMPRSS4 | QVQLVQSGAEVKKPGASVKVSCKASGYTFSRYFMHWVRQAPGQGLEWMGWINPNSGGTNYAQKFQGR |
| Ab46 scFv | VTMTRDTSTSTVYMELSSLRSEDTAVYYCARVRIGWLQSPPLYWGQGTLVTVSSGGGGSGGGGSGGG |
| (VH-VL) | GSDIQMTQSPSSLSASVGDRVTITCRASQSISTWLAWYQQKPGKAPKLLIYAASSLQSGVPSRFSGS |
| | GSGTDFTLTISSLQPEDFATYYCQQSYGFPWTFGQGTKVEIK (SEQ ID NO: 549) |
| TMPRSS4 | QVQLVQSGAEVKKPGASVKVSCKASGYTFTGYFMHWVRQAPGQGLEWMGWMNPNSGNTGYAQKFQGR |
| Ab47 scFv | VTMTRDTSTSTVYMELSSLRSEDTAVYYCVRGRTWIQSSLGYWGQGTLVTVSSGGGGSGGGGSGGGG |
| (VH-VL) | SDIQMTQSPSSLSASVGDRVTITCRASQSISSYLNWYQQKPGKAPKLLIYAASSLQSGVPSRFSGSG |
| | SGTDFTLTISSLQPEDFATYYCQQSYSTPLTFGGGTKVEIK (SEQ ID NO: 550) |
| TMPRSS4 | QVQLVQSGAEVKKPGASVKVSCKASGYTFTGYYLHWVRQAPGQGLEWMGWISAYNGNTNYAQNLQGR |
| Ab48 scFv | VTMTRDTSTSTVYMELSSLRSEDTAVYYCARHSYSGSYSTLPYYGMDVWGQGTTVTVSSGGGGSGGG |
| (VH-VL) | GSGGGGSDIQMTQSPSSLSASVGDRVTITCRASQGISNYLAWYQQKPGKAPKLLIYTASTLFPGVPS |
| | RFSGSGSGTDFTLTISSLQPEDFATYYCQQSYSIPLTFGGGTKVEIK (SEQ ID NO: 551) |
| TMPRSS4 | QVQLVQSGAEVKKPGASVKVSCKASGYTFTGYYMHWVRQAPGQGLEWMGRINPNSGGTNYAQKFQGR |
| Ab49 scFv | VTMTRDTSTSTVYMELSSLRSEDTAVYYCARERAGYSSGQFDYWGQGTLVTVSSGGGGSGGGGSGGG |
| (VH-VL) | GSDIQMTQSPSSLSASVGDRVTITCRASQSISNWLAWYQQKPGKAPKLLIYAASTLQNGVPSRFSGS |
| | GSGTDFTLTISSLQPEDFATYYCQQSYTFPITFGQGTKVEIK (SEQ ID NO: 552) |
| TMPRSS4 | QVQLVQSGAEVKKPGASVKVSCKASGYTFTGYYMHWVRQAPGQGLEWMGWINPNSGGTHYAQKFQGR |
| Ab50 scFv | VTMTRDTSTSTVYMELSSLRSEDTAVYYCARVRIGWLQSPPLYWGQGTLVTVSSGGGGSGGGGSGGG |
| (VH-VL) | GSDIQMTQSPSSLSASVGDRVTITCRASQGISNYLAWYQQKPGKAPKLLIYATSRLQSGVPSRFSGS |
| | GSGTDFTLTISSLQPEDFATYYCQQSYKTPLTFGGGTKVEIK (SEQ ID NO: 553) |
| TMPRSS4 | QVQLVQSGAEVKKPGASVKVSCKASGYTFTNYYMHWVRQAPGQGLEWMGWINPKSGGTSYAQKFQGR |
| Ab51 scFv | VTMTRDTSTSTVYMELSSLRSEDTAVYYCASGKQWLVGGRFDYWGQGTLVTVSSGGGGSGGGGSGGG |
| (VH-VL) | GSDIQMTQSPSSLSASVGDRVTITCRASQSISSYLNWYQQKPGKAPKLLIYAASSLQSGVPSRFSGS |
| | GSGTDFTLTISSLQPEDFATYYCQQSYSTPLTFGGGTKVEIK (SEQ ID NO: 554) |
| TMPRSS4 | QVQLVQSGAEVKKPGASVKVSCKASGYTFTRYYIHWVRQAPGQGLEWMGWMNPNSGNTGFAQKLQGR |
| Ab52 scFv | VTMTRDTSTSTVYMELSSLRSEDTAVYYCARGPFPRGRLDLWGQGTLVTVSSGGGGSGGGGSGGGGS |
| (VH-VL) | DIQMTQSPSSLSASVGDRVTITCRASQGISRWLGWYQQKPGKAPKLLIYGASNLQTGVPSRFSGSGS |
| | GTDFTLTISSLQPEDFATYYCQQSYSSPRTFGQGTKVEIK (SEQ ID NO: 555) |
| TMPRSS4 | QVQLVQSGAEVKKPGASVKVSCKASGYTFTRYYMHWVRQAPGQGLEWMGIINPTGGSTSYAQKFQGR |
| Ab53 scFv | VTMTRDTSTSTVYMELSSLRSEDTAVYYCARGRTWIQSSLGYWGQGTLVTVSSGGGGSGGGGSGGGG |
| (VH-VL) | SDIQMTQSPSSLSASVGDRVTITCRASRSINRWLAWYQQKPGKAPKLLIYGASTLQSGVPSRFSGSG |
| | SGTDFTLTISSLQPEDFATYYCQQSYSTPTFGGGTKVEIK (SEQ ID NO: 556) |
| TMPRSS4 | QVQLVQSGAEVKKPGASVKVSCKASGYTFTSYYMQWVRQAPGQGLEWMGWMNPNSGNTGYAQKFQGR |
| Ab54 scFv | VTMTRDTSTSTVYMELSSLRSEDTAVYYCARVRIGWLQSPPLYWGQGTLVTVSSGGGGSGGGGSGGG |
| (VH-VL) | GSDIQMTQSPSSLSASVGDRVTITCRASQGISNYLAWYQQKPGKAPKLLIYAASSLQSGVPSRFSGS |
| | GSGTDFTLTISSLQPEDFATYYCQQSYSIPFTFGPGTKVDIK (SEQ ID NO: 557) |
| TMPRSS4 | QVQLVQSGAEVKKPGASVKVSCKASGYTFTTYYMHWVRQAPGQGLEWMGIINPSGGSTSYAQKFQGR |
| Ab55 scFv | VTMTRDTSTSTVYMELSSLRSEDTAVYYCARGRSWYRSNVDYWGQGTLVTVSSGGGGSGGGGSGGGG |
| (VH-VL) | SDIQMTQSPSSLSASVGDRVTITCRASQSISSWLAWYQQKPGKAPKLLIYAASSLQSGVPSRFSGSG |
| | SGTDFTLTISSLQPEDFATYYCQQSYSTPRTFGQGTRLEIK (SEQ ID NO: 558) |
| TMPRSS4 | QVQLVQSGAEVKKPGASVKVSCKASGHTFTRYYMHWVRQAPGQGLEWMGWINPNSGNTGDAQKFQGR |
| Ab56 scFv | VTMTRDTSTSTVYMELSSLRSEDTAVYYCARDRGIVVVPAAIGGMDVWGQGTMVTVSSGGGGSGGGG |
| (VH-VL) | SGGGGSDIVMTQSPLSLPVTPGEPASISCRSSQSLLHSNGYNYLDWYLQKPGQSPQLLIYLGSNRAS |
| | GVPDRFSGSGSGTDFTLKISRVEAEDVGVYYCMQALQTPITFGQGTRLEIK (SEQ ID NO: 559) |
| TMPRSS4 | QVQLVQSGAEVKKPGASVKVSCKASGYTFTGYFMHWVRQAPGQGLEWMGRINPNSGGTNYAQKFQGR |
| Ab57 scFv | VTMTRDTSTSTVYMELSSLRSEDTAVYYCARGKGRYFDLWGRGTLVTVSSGGGGSGGGGSGGGGSDI |
| (VH-VL) | VMTQSPLSLPVTPGEPASISCRSSQSLLHSNGYNYLDWYLQKPGQSPQLLIYLGSNRASGVPDRFSG |
| | SGSGTDFTLKISRVEAEDVGVYYCMQGTHWPITFGQGTRLEIK (SEQ ID NO: 560) |
| TMPRSS4 | QVQLVQSGAEVKKPGASVKVSCKASGYTFTRYYLHWVRQAPGQGLEWMGWVSAYNGNTNYAQKFQGR |
| Ab58 scFv | VTMTRDTSTSTVYMELSSLRSEDTAVYYCARGYCSGGSCYWFDPWGQGTLVTVSSGGGGSGGGGSGG |
| (VH-VL) | GGSDIVMTQSPLSLPVTPGEPASISCRSSQSLLHSNGYNYLDWYLQKPGQSPQLLIYLGSNRASGVP |
| | DRFSGSGSGTDFTLKISRVEAEDVGVYYCMQALQTPLTFGQGTKVEIK (SEQ ID NO: 561) |
| TMPRSS4 | QVQLVQSGAEVKKPGASVKVSCKASGGTFSSYTLSWVRQAPGQGLEWMGWIHPKSGVTKNAQKFQGR |
| Ab59 scFv | VTMTRDTSTSTVYMELSSLRSEDTAVYYCARGWVYGRMDAWGQGTTVTVSSGGGGSGGGGSGGGGSE |
| (VH-VL) | IVMTQSPATLSVSPGERATLSCRASQSVSSNYLAWYQQKPGQAPRLLIYGASTRATGIPARFSGSGS |
| | GTEFTLTISSLQSEDFAVYYCQQYGTLPYTFGQGTKVEIK (SEQ ID NO: 562) |
| TMPRSS4 | QVQLVQSGAEVKKPGASVKVSCKASGYSFTTYYIHWVRQAPGQGLEWMGIINPSGGSTSYAQKFQGR |
| Ab60 scFv | VTMTRDTSTSTVYMELSSLRSEDTAVYYCARGGYYGSGYNSVGYWGPGTLVTVSSGGGGSGGGGSGG |
| (VH-VL) | GGSEIVMTQSPATLSVSPGERATLSCRASQSVSSNTLAWYQQKPGQAPRLLIYGASTRATGIPARFS |
| | GSGSGTEFTLTISSLQSEDFAVYYCQQYGSSPLTFGPGTKVDIK (SEQ ID NO: 563) |
| TMPRSS4 | QVQLVQSGAEVKKPGASVKVSCKASGYTFTNYYMHWVRQAPGQGLEWMGWMNPNSGNTGYAQNLQGR |
| Ab61 scFv | VTMTRDTSTSTVYMELSSLRSEDTAVYYCARGRTWFRSGMDVWGQGTTVTVSSGGGGSGGGGSGGGG |
| (VH-VL) | SEIVMTQSPATLSVSPGERATLSCRASQSVSSYLAWYQQKPGQAPRLLIYGASTRATGIPARFSGSG |
| | SGTEFTLTISSLQSEDFAVYYCQQYDISVTFGPGTKVDIK (SEQ ID NO: 564) |
| TMPRSS4 | QVQLVQSGAEVKKPGASVKVSCKASGYTFTDYYIHWVRQAPGQGLEWMGWISTYNGNTNYAQKLQGR |
| Ab62 scFv | VTMTRDTSTSTVYMELSSLRSEDTAVYYCARGMVRGMDVWGQGTMVTVSSGGGGSGGGGSGGGGSDI |
| (VH-VL) | VMTQSPDSLAVSLGERATINCKSSQSVLYSSNNKNYLAWYQQKPGQPPKLLIYWASTRESGVPDRFS |
| | GSGSGTDFTLTISSLQAEDVAVYYCQQYYTTPWTFGQGTRLEIK (SEQ ID NO: 565) |
| TMPRSS4 | QVQLVQSGAEVKKPGASVKVSCKASGYTFTGYRMHWVRQAPGQGLEWMGVINPNTGTARFAQKFQGR |
| Ab63 scFv | VTMTRDTSTSTVYMELSSLRSEDTAVYYCASVGVYWYFDLWGRGTLVTVSSGGGGSGGGGSGGGGSD |
| (VH-VL) | IVMTQSPDSLAVSLGERATINCKSSQSVLYSSNNKNYLAWYQQKPGQPPKLLIYWASTRESGVPDRF |
| | SGSGSGTDFTLTISSLQAEDVAVYYCQQYYSAPLTFGGGTKVEIK (SEQ ID NO: 566) |
| TMPRSS4 | QVQLVQSGAEVKKPGASVKVSCKASGYTFTGYYMHWVRQAPGQGLEWMGMINPSGGGTTYAQKFQGR |
| Ab64 scFv | VTMTRDTSTSTVYMELSSLRSEDTAVYYCARDRRSMITFRTDYWGQGTLVTVSSGGGGSGGGGSGGG |
| (VH-VL) | GSDIVMTQSPDSLAVSLGERATINCKSSQSVLYSSNNKNYLAWYQQKPGQPPKLLIYWASTRESGVP |
| | DRFSGSGSGTDFTLTISSLQAEDVAVYYCQQYYSTPYTFGQGTKLEIK (SEQ ID NO: 567) |
| TMPRSS4 | QVQLVQSGAEVKKPGASVKVSCKASGYTFTGYYMHWVRQAPGQGLEWMGWMNPNSGNTGYAQKFQGR |
| Ab65 scFv | VTMTRDTSTSTVYMELSSLRSEDTAVYYCAGRKWLGLDFYNWEDPWGQGTLVTVSSGGGGSGGGGSG |
| (VH-VL) | GGGSDIVMTQSPDSLAVSLGERATINCKSSQSVLYSSNNKNYLAWYQQKPGQPPKLLIYWASTRQSG |
| | VPDRFSGSGSGTDFTLTISSLQAEDVAVYYCQQYYSTPWTFGQGTKVEIK (SEQ ID NO: 568) |
| TMPRSS4 | QVQLVQSGAEVKKPGSSVKVSCKASGGTFSSYAISWVRQAPGQGLEWVGGIMPIFGTANYAQKFQGR |
| Ab66 scFv | VTITADESPSTAYMELSSLRSEDTAVYYCATGRRELLNWGQGTLVTVSSGGGGSGGGGSGGGGSEIV |
| (VH-VL) | MTQSPATLSVSPGERATLSCRASQSVNSRFLAWYQQKPGQAPRLLIYGASTRATGIPARFSGSGSGT |
| | EFTLTISSLQSEDFAVYYCMQGTHWPYTFGQGTKVEIK (SEQ ID NO: 569) |
| TMPRSS4 | QVQLVQSGAEVKKPGSSVKVSCKASGYTFTSYDINWVRQAPGQGLEWMGGIIPIFGTANYAQKFQGR |
| Ab67 scFv | VTITADESTSTAYMELSSLRSEDTAVYYCATTPGDAFDIWGQGTMVTVSSGGGGSGGGGSGGGGSDI |
| (VH-VL) | VMTQSPDSLAVSLGERATINCKSSQSVLYSSNNKNYLAWYQQKPGQPPKLLIYWASTRESGVPDRFS |
| | GSGSGTDFTLTISSLQAEDVAVYYCQQYSDTPLTFGQGTKVEIK (SEQ ID NO: 570) |
| TMPRSS4 | EVQLLESGGGLVKPGGSLRLSCAASGFTFSSSWMHWVRQAPGKGLEWVSAIGTAGDTYYPGSVKGRF |
| Ab68 scFv | TISRDDSKNTLYLQMNSLKTEDTAVYYCARVRLGHFDLWGRGTLVTVSSGGGGSGGGGSGGGGSDIQ |
| (VH-VL) | MTQSPSSLSASVGDRVTITCRASQSISRYLNWYQQKPGKAPKLLIYAASSLQSGVPSRFSGSGSGTD |
| | FTLTISSLQPEDFATYYCQQSYSNPPTFGQGTKLEIK (SEQ ID NO: 571) |
| TMPRSS4 | EVQLLESGGGLVQHGGSLRLSCAASGFAFSSYVLHWVRQAPGKGLEWVSSISSSSSYIYYADSVKGR |
| Ab69 scFv | FTISRDNSKNTLYLQMNSLRAEDTAVYYCARGDRYPGLPNYWGQGTLVTVSSGGGGSGGGGSGGGGS |
| (VH-VL) | DIQMTQSPSSLSASVGDRVTITCRASQGISSWLAWYQQKPGKAPKLLIYQASNKDTGVPSRFSGSGS |
| | GTDFTLTISSLQPEDFATYYCQQSYRIPWTFGQGTKVEIK (SEQ ID NO: 572) |
| TMPRSS4 | EVQLLESGGGLVQPGGSLRLSCAASGFAFSGTWMQWVRQAPGKGLEWVSDISGSSRDTNYADSVKGR |
| Ab70 scFv | FTISRDNSKNTLYLQMNSLRAEDTAVYYCAKDHWDSYGYLDYWGQGTLVTVSSGGGGSGGGGSGGGG |
| (VH-VL) | SDIQMTQSPSSLSASVGDRVTITCRASQSISGWLAWYQQKPGKAPKLLIYAASTLRDGVPSRFSGSG |
| | SGTDFTLTISSLQPEDFATYYCQQANSFPLTFGQGTKVEIK (SEQ ID NO: 573) |
| TMPRSS4 | EVQLLESGGGLVQPGGSLRLSCAASGFMFDYYAMHWVRQAPGKGLEWVSLISYDGRNKYYADSVKGR |
| Ab71 scFv | FTISRDNSKNTLYLQMNSLRAEDTAVYYCARPGSYSRFQHWGQGTLVTVSSGGGGSGGGGSGGGGSD |
| (VH-VL) | IQMTQSPSSLSASVGDRVTITCRASQGISNNLNWYQQKPGKAPKLLIYAASSLQSGVPSRFSGSGSG |
| | TDFTLTISSLQPEDFATYYCQQANNFPITFGQGTKVEIK (SEQ ID NO: 574) |
| TMPRSS4 | EVQLLESGGGLVQPGGSLRLSCAASGFTFGAYVMHWVRQAPGKGLEWVSSISGGSTYYADSVKGRFT |
| Ab72 scFv | ISRDNSKNTLYLQMNSLRAEDTAVYYCARHPVRGVIGAGWFDPWGQGTLVTVSSGGGGSGGGGSGGG |
| (VH-VL) | GSDIQMTQSPSSLSASVGDRVTITCRASQNISRWLAWYQQKPGKAPKLLIYAASSLQSGVPSRFSGS |
| | GSGTDFTLTISSLQPEDFATYYCQQAISFPLTFGGGTKVEIK (SEQ ID NO: 575) |
| TMPRSS4 | EVQLLESGGGLVQPGGSLRLSCAASGFTFSSYAMHWVRQAPGKGLEWLAVISEDGSIRHYADSVKGR |
| Ab73 scFv | FTISRDNSKNTLYLQMNSLRAEDTAVYYCAKPKASSGPRLIDYWGQGTLVTVSSGGGGSGGGGSGGG |
| (VH-VL) | GSDIQMTQSPSSLSASVGDRVTITCRASQGISNSLAWYQQKPGKAPKLLIYSAVNLQSGVPSRFSGS |
| | GSGTDFTLTISSLQPEDFATYYCQQANSFPLTFGGGTKVEIK (SEQ ID NO: 576) |
| TMPRSS4 | EVQLLESGGGLVQPGGSLRLSCAASGFTFSSYAMHWVRQAPGKGLEWVSSISSSSTYIHYADSVKGR |
| Ab74 scFv | FTISRDNSKNTLYLQMNSLRAEDTAVYYCARVGRYYGSGSSLVDYWGQGTLVTVSSGGGGSGGGGSG |
| (VH-VL) | GGGSDIQMTQSPSSLSASVGDRVTITCRASQSVSSWLAWYQQKPGKAPKLLIYDASSLQSGVPSRFS |
| | GSGSGTDFTLTISSLQPEDFATYYCQQAKSFPPTFGQGTKVEIK (SEQ ID NO: 577) |
| TMPRSS4 | EVQLLESGGGLVQPGGSLRLSCAASGFTFSSYAMSWVRQAPGKGLEWVSSISSASSYKYYADSVKGR |
| Ab75 scFv | FTISRDNSKNTLYLQMNSLRAEDTAVYYCARDIYSSGWRGYYYYGMDVWGQGTTVTVSSGGGGSGGG |
| (VH-VL) | GSGGGGSDIQMTQSPSSLSASVGDRVTITCRASQGIRNDLNWYQQKPGKAPKLLIYAATRLQSGVPS |
| | RFSGSGSGTDFTLTISSLQPEDFATYYCQQAHSFPYSFGQGTRLEIK (SEQ ID NO: 578) |
| TMPRSS4 | EVQLLESGGGLVQPGGSLRLSCAASGFTFSSYAMSWVRQAPGKGLEWVSAISGSGGNAYYADSVKGR |
| Ab76 scFv | FTISRDNAKNSLYLQMNSLRAEDTAVYYCAKNSWGSYRPRAFDIWGQGTMVTVSSGGGGSGGGGSGG |
| (VH-VL) | GGSDIVMTQSPDSLAVSLGERATINCKSSQSVLYSSNNKNYLAWYQQKPGQPPKLLIYWASTRASGV |
| | PDRFSGSGSGTDFTLTISSLQAEDVAVYYCQQYLSLPYTFGQGTKVEIK (SEQ ID NO: 579) |
| TMPRSS4 | QVQLVQSGAEVKKPGASVKVSCKASGNIFTAQYMHWVRQAPGQGLEWMGWMNPNTVYTGSAQKFQGR |
| Ab77 scFv | VTMTRDTSTSTVYMELSSLRSEDTAVYYCARDWVGDGYNSFDYWGQGTLVTVSSGGGGSGGGGSGGG |
| (VH-VL) | GSDIVMTQSPDSLAVSLGERATINCKSSQSVLYSSNNKNYLAWYQQKPGQPPKLLIYWASTRESGVP |
| | DRFSGSGSGTDFTLTISSLQAEDVAVYYCQQYYTTPFTFGPGTKVDIK (SEQ ID NO: 580) |
| TMPRSS4 | EVQLLESGGGLVQPGGSLRLSCAASGFTFSSYGMNWVRQAPGKGLEWVSAISGSGGRTYYADSVKGR |
| Ab78 scFv | FTISRDNAKNSLYLQMNSLRAEDTAVYYCAKGTYYSSPKYSFDYWGQGTLVTVSSGGGGSGGGGSGG |
| (VH-VL) | GGSDIQMTQSPSSLSASVGDRVTITCRASQDIKNFLAWYQQKPGKAPKLLIYAASSLQSGVPSRFSG |
| | SGSGTDFTLTISSLQPEDFATYYCQQSYSTPWTFGQGTKLEIK (SEQ ID NO: 581) |
| TMPRSS4 | EVQLLESGGGLVQPGGSLRLSCAASGLTFSSYQMSSVSQAPGKGLEWVSYISSAANTVYYADSVKGR |
| Ab79 scFv | FTISRDNSKNTLYLQMNSLRAEDTAVYYCAREDESRSPYCSGGSCYRAEYFQHWGQGTLVTVSSGGG |
| (VH-VL) | GSGGGGSGGGGSDIQMTQSPSSLSASVGDRVTITCRASQGISNYLAWYQQKPGKAPKLLIYAASSLQ |
| | SGVPSRFSGSGSGTDFTLTISSLQPEDFATYYCQQSYSTPLTFGPGTKVDIK (SEQ ID NO: 582) |

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 504, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 504.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 505, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 505.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 506, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 506.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 507, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 507.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 508, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 508.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 509, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 509.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 510, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 510.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 511, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 511.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 512, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 512.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 513, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 513.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 514, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 514.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 515, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 515.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 516, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 516.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 517, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 517.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 518, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 518.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 519, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 519.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 520, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 520.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 521, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 521.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 522, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 522.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 523, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 523.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 524, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 524.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 525, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 525.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 526, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 526.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 527, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 527.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 528, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 528.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 529, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 529.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 530, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 530.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 531, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 531.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 532, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 532.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 533, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 533.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 534, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 534.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 535, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 535.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 536, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 536.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 537, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 537.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 538, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 538.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 539, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 539.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 540, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 540.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 541, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 541.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 542, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 542.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 543, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 543.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 544, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 544.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 545, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 545.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 546, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 546.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 547, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 547.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 548, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 548.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 549, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 549.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 550, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 550.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 551, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 551.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 552, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 552.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 553, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 553.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 554, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 554.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 555, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 555.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 556, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 556.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 557, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 557.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 558, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 558.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 559, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 559.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 560, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 560.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 561, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 561.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 562, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 562.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 563, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 563.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 564, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 564.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 565, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 565.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 566, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 566.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 567, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 567.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 568, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 568.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 569, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 569.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 570, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 570.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 571, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 571.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 572, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 572.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 573, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 573.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 574, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 574.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 575, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 575.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 576, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 576.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 577, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 577.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 578, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 578.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 579, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 579.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 580, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 580.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 581, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 581.

In some embodiments, the antibody or antigen-binding fragment that binds to TMPRSS4 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the amino acid sequence set forth in SEQ ID NO: 582, and optionally comprises VH CDRs and VL CDRs that are 100% identical to those therein. In some embodiments, the antibody or antigen-binding fragment that binds to TMPRSS4 comprises an scFv comprising the amino acid sequence set forth in SEQ ID NO: 582.

Table 5 provides exemplary nucleic acid sequences encoding VH and VL domains that, in combination, bind to TMPRSS4. In some embodiments, the VH and the VL of the antibody or antigen-binding fragment that binds to TMPRSS4 are each encoded by a sequence set forth in Table 6.

TABLE 6
TMPRSS4 VH and VL Nucleic Acid Sequences

| Clone | VL Nucleic Acid Sequence | VH Nucleic Acid Sequence |
|---|---|---|
| TMPRSS4 | CAAGTGCAGCTCGTTCAGTCTGGGGCGGAGGT | GACATTCAGATGACTCAGTCGCCAAGTTCACT |
| Ab1 | TAAAAAACCGGGCTCATCTGTGAAAGTGTCTT | GTCAGCTTCCGTCGGCGATAGAGTCACTATTA |
| | GCAAGGCCTCCGGCTATACCTTCACCGATTAC | CTTGTCAAGCATCCCAGGATATATCCAACTAT |
| | TACATGCATTGGGTCCGGCAGGCACCCGGGCA | TTGAACTGGTACCAGCAGAAGCCCGGCAAGGC |
| | GGGACTTGAGTGGATGGGCGGGATTATCCCAA | ACCCAAGCTGCTGATATACAAGGCCTCCTCTC |
| | TCTTTGGGACTGCAAATTATGCTCAGAAATTC | TGGAATCAGGCGTGCCTAGCCGATTTTCTGGG |
| | CAAGGGCGCGTAACTATTACCGCCGACGAAAG | TCTGGGAGTGGCACAGATTTCACGCTGACAAT |
| | CACAAGCACCGCGTACATGGAGCTATCGAGCC | CAGCTCCTTACAGCCTGAGGACTTCGCCACCT |
| | TCCGTAGCGAGGACACCGCTGTGTACTATTGC | ATTATTGTCAACAAAGCAGCAGGATCCCTCCA |
| | GCCAAGGAAGGCGCTAATGGCTACTGGGGTCA | ACCTTTGGGCAGGGAACGAAAGTTGAAATCAA |
| | GGGAACATTGGTAACAGTGTCCAGT (SEQ ID | G (SEQ ID NO: 584) |
| | NO: 583) | |
| TMPRSS4 | GAAGTGCAGCTCCTAGAGTCTGGCGGCGGGCT | GACATACAGATGACACAGAGCCCGAGTTCCCT |
| Ab2 | GGTTCAACCTGGTGGGTCATTGCGGCTGAGCT | GTCCGCTAGTGTTGGGGACAGAGTCACCATTA |
| | GCGCTGCCAGTGGGTTCACCTTTTCAGATTAC | CATGCAGGGCCTCGCAGAGCACTAACAACTAT |
| | TACATGTCATGGGTCCGGCAGGCTCCCGGGAA | GTGAATTGGTATCAGCAAAAACCCGGCAAAGC |
| | GGGCCTTGAGTGGGTGTCCTACATCTCAGGTT | ACCAAAACTGCTGATATACGCGGCCTCCTCCC |
| | CTGGCGATGCAATCTACTATGCTGACTCTGTA | TCCAATCTGGTGTGCCTTCTCGTTTTAGCGGG |
| | AAGGGCCGCTTCACCATTAGCCGCGACAATTC | AGTGGGTCGGGCACTGATTTCACGCTCACAAT |
| | AAAAAATACTCTGTACCTCCAGATGAACAGCC | CAGTTCCTTGCAGCCCGAGGACTTTGCCACCT |
| | TTCGAGCCGAAGATACTGCCGTGTACTATTGT | ACTATTGTCAACAGAGCTATAGCATTCCTTTA |
| | GCAAGGGATAGGTCTGATTGCGGTGGGGACGA | ACCTTCGGACCAGGGACAAAGGTCGATATCAA |
| | CAGATTCCTGTGTGACGGTTATTTTGACCTGT | G (SEQ ID NO: 586) |
| | GGGGAAGAGGCACGCTTGTGTCCTTGTCT | |
| | (SEQ ID NO: 585) | |
| TMPRSS4 | CAGGTTCAGCTCGTCCAGTCTGGGGCAGAAGT | GACATAGTTATGACTCAGTCACCACTGTCCCT |
| Ab3 | GAAAAAACCTGGAAGCAGCGTAAAAGTCTCCT | CCCTGTGACTCCTGGCGAGCCCGCCTCAATCT |
| | GCAAGGCCTCCGGATATACGTTCACCTCTTAT | CCTGTAGGAGTTCAGGGTCCCTGCTGCATTCT |
| | GACATTAACTGGGTGCGGCAGGCACCCGGCCA | AATGGCTACAATTACCTGGACTGGTACCTGCA |
| | GGGCCTTGAGTGGATGGGCGGGATTATCCCAA | AAAGCCCGGGCAATCACCGCAGCTATTGATCT |
| | TTTTTGGGACCACAAAGTTTGCTCAGAAGTTT | ACGCGGCAAGTAGCTTACAGAGCGGCGTGCCT |
| | CAGGGCCGCGTGACCATAACCGCTGATGAAAG | GACCGATTCTCTGGGTCAGGGAGCGGTACTGA |
| | TACAAGCACAGCTTACATGGAGCTCTCGAGTT | TTTCACCCTGAAGATCTCTAGAGTTGAAGCCG |
| | TGAGGAGCGAGGATACTGCCGTGTACTATTGT | AGGACGTCGGTGTCTACTATTGCATGCAAGGG |
| | GCCAGAGATTGGTATTCCTCGAGCTGGTATAA | ACCCACTGGCCCGGCACGTTTGGCCAAGGGAC |
| | CGGAGATCGTGGGGATTGGTTCGACCCATGGG | AAAAGTGGAAATCAAG (SEQ ID NO: 588) |
| | GTCAGGGAACACTTGTAACTGTGTCCTCC | |
| | (SEQ ID NO: 587) | |
| TMPRSS4 | CAAGTGCAGCTCGTCCAGAGTGGTGCCGAAGT | GACATTGTTATGACACAATCACCATTGAGCCT |
| Ab4 | GAAAAAACCTGGGGCATCCGTAAAGGTGTCCT | CCCAGTGACTCCAGGCGAGCCCGCCTCCATCT |
| | GCAAGGCCTCAGGCTATTTTTTCACTACATAC | CGTGTCGAAGTTCCCAGTCCCTTCTGCACAGC |
| | TACTTACATTGGGTTCGGCAGGCTCCCGGGCA | AACGGCTATAACTACTTAGACTGGTACTTGCA |
| | GGGCCTGGAATGGATGGGCGTCATAAATCCGA | GAAGCCCGGCCAGAGTCCTCAGCTCCTAATCT |
| | ATAGTCGCCTGACTAGCTACGCAGAATCCTTT | ACGCAGCGAGCACGCTCCAGTCAGGCGTGCCC |
| | CAGGGAAGGGTCACAATGACTAGAGATACCAG | GATAGGTTTTCTGGCAGCGGGTCTGGGACCGA |
| | CACATCTACCGTCTATATGGAGCTGAGCAGTC | TTTCACGCTGAAGATTTCTCGGGTTGAAGCTG |
| | TGCGTAGCGAGGACACCGCTGTGTACTATTGT | AGGACGTGGGCGTCTACTATTGCATGCAAGGG |
| | GAGAGAGAAATGTTCCCATCGAGCTATGGGAT | ACCCACTGGCCTCCTACCTTCGGCCAAGGGAC |
| | TGATGTGTGGGGTCAGGGAACCACGGTGACAG | AAAACTTGAGATCAAG (SEQ ID NO: 590) |
| | TTTCAAGC (SEQ ID NO: 589) | |
| TMPRSS4 | CAAGTGCAACTGGTTCAGTCTGGGGCGGAAGT | GAGATTGTTATGACACAGTCGCCAGCAACCCT |
| Ab5 | CAAGAAGCCAGGCTCTAGCGTAAAAGTCTCGT | GAGTGTCAGCCCGGGCGAGAGGGCAACCCTCA |
| | GCAAAGCCTCCGGGTACACTTTTACGAGCTAT | GTTGTAGAGCTTCTCAGAGCGTGTCCTCCAAC |
| | TACATGCATTGGGTGCGGCAGGCTCCCGGGCA | CTAGCATGGTATCAACAGAAGCCCGGCCAGGC |
| | GGGCCTGGAATGGATGGGTCGGATCATTCCTA | ACCCAGGTTGCTCATCTACGGCGCCTCAACAA |
| | TCCTGGGCGCCACCGATTATGCACAGAAATTC | GAGCCACCGGGATTCCTGCCCGTTTCTCTGGG |
| | CAAGGGCGCGTAACTATAACTGCCGACGAGAG | AGCGGGAGCGGGACAGAGTTTACGCTTACAAT |
| | CACATCAACCGCCTACATGGAGCTCAGCTCCC | AAGTAGTTTACAGTCAGAAGATTTCGCTGTGT |
| | TGCGATCCGAGGACACCGCTGTGTACTATTGC | ACTATTGTCAACAGTACTACTCTCCTTTCCCA |
| | GCCAGGGCTGGCTATTCCTCCATCGCCGCGCG | TTGACTTTTGGTGGCGGCACAAAGGTGGAAAT |
| | CCCGGCTTTTTGGGGTCAGGGAACTCTTGTTA | CAAG (SEQ ID NO: 592) |
| | CCGTGTCTAGT (SEQ ID NO: 591) | |
| TMPRSS4 | CAAGTGCAGCTCGTCCAGTCTGGAGCCGAAGT | GACATTGTCATGACTCAGTCTCCCGATAGTCT |
| Ab6 | GAAAAAACCTGGGGCGTCTGTAAAAGTGTCCT | TGCTGTGTCCCTGGGAGAAAGGGCAACAATTA |
| | GCAAGGCCTCCGGCTATACTTTCACGAGCTAT | ATTGTAAGTCCTCCCAGTCTGTGCTGTACTCC |
| | TACATGCATTGGGTGCGGCAGGCACCAGGACA | AGCAACAACAAAAATTACTTGGCATGGTATCA |
| | AGGGTTGGAGTGGCTCGGGATAATCAACCCAT | GCAAAAGCCCGGTCAGCCGCCCAAACTGCTGA |
| | CAGATTACACCACAAGCTACGCGCAGAAGTTT | TCTACTGGGCTTCAACCCGGGAAAGCGGCGTG |
| | CAGGGCCGCGTCACCATGACCAGGGATACTTC | CCTGATCGTTTCAGCGGGAGCGGGTCTGGGAC |
| | TACAAGCACCGTCTACATGGAACTCTCGAGTC | AGACTTTACACTGACCATATCCAGCTTACAGG |
| | TAAGAAGTGAGGATACTGCTGTGTATTACTGC | CTGAGGACGTTGCCATCTACTATTGTCAACAA |
| | GCCCGAGTGGCCTCTTCATCCTGGTATCCAGG | TACTATGCCATTCCTTGGACTTTCGGGCAGGG |
| | CGACGAGAATTGGTATTTTGACCTGTGGGGCA | AACGAAGGTTGAAATCAAG (SEQ ID NO: 594) |
| | GAGGCACACTTGTCACCGTTAGCTCA (SEQ | |
| | ID NO: 593) | |
| TMPRSS4 | CAGGTCCAGCTTGTCCAGTCCGGCGCCGAAGT | GAAATTGTGATGACTCAGAGTCCTGCTACTCT |
| Ab7 | CAAAAAACCCGGCGCGAGTGTCAAAGTGTCCT | GAGTGTGTCTCCTGGTGAGAGGGCAACCCTCA |
| | GCAAGGCCTCCGGCTATACTTTTTCCCGGTAC | GTTGTAGAGCTTCGCAGAGAGTATCTAACAAC |
| | TTCATGCATTGGGTGCGGCAGGCACCAGGCCA | TATCTCGCATGGTATCAGCAAAAGCCCGGGCA |
| | AGGGTTGGAGTGGGTTGGCTGGATTAATCCCA | GGCTCCTAGGCTGCTTATCTACGGCGCGTCAA |
| | ACTCCGGGAATACCGGCTATGCCCAAAAGTTT | CCCGGGCCTCTGGCATTCCGGCCCGTTTTTCT |
| | CAGGGCCGCGTAACCATGACTCGAGATACATC | GGGAGCGGATCAGGGACTGAGTTCACACTAAC |
| | TACATCCACTGTCTACATGGAGCTGTCTAGCC | AATATCCAGCTTACAGAGCGAGGACTTCGCCG |
| | TGCGCAGCGAAGACACCGCAGTGTACTATTGC | TGTACTATTGTCAACAGTACGGGAGCACACCA |
| | GCTAGGGTTGTTACCGGTGGCAGACTGGATGT | TACACCTTCGGCCAAGGGACGAAGGTTGAAAT |
| | GTGGGGACAAGGCACAACCGTGACAGTGTCAA | CAAG (SEQ ID NO: 596) |
| | GC (SEQ ID NO: 595) | |
| TMPRSS4 | CAAGTACAGTTGGTCCAGAGCGGCGCAGAAGT | GACATTCAGATGACTCAGTCGCCCTCTTCACT |
| Ab8 | CAAGAAGCCTGGGGCGTCAGTGAAAGTGTCCT | GTCAGCTTCGGTTGGGGATAGAGTTACTATTA |
| | GCAAAGCCTCCGGGTATCGGTTCACAAGCCAG | CATGCCGAGCCTCTCAGAGTATCTCCTCCTGG |
| | TACATGCATTGGGTCCGGCAGGCGCCAGGCCA | CTGGCCTGGTATCAGCAAAAGCCGGGCAAAGC |
| | GGGTTTGGAGTGGATGGGGATAATTAATCCTA | ACCCAAGCTGCTGATCTACGGAGCCAGTTCCC |
| | GCGGCGGTAGTACGAGTTACGCGCAGAAGTTC | TACAGAGCGGAGTGCCCTCTCGCTTCAGCGGG |
| | CAGGGCCGCGTTACCATGACTCGTGACACCTC | AGTGGCTCTGGGACCGACTTCACACTTACAAT |
| | TACCAGCACCGTCTACATGGAACTCTCCTCCC | AAGCAGTTTACAGCCCGAAGATTTTGCAACGT |
| | TGAGGAGCGAGGATACCGCCGTGTATTACTGT | ATTATTGTCAACAAGCTAACTCATTTCCACCT |
| | GCTAGAGGCAGGATCGCTGTGGCAGGCCACCC | ACCTTTGGCGGAGGAACAAAAGTGGAGATCAA |
| | ACTCGGCTACTGGGGTCAAGGGACTCTTGTAA | G (SEQ ID NO: 598) |
| | CAGTGTCCAGT (SEQ ID NO: 597) | |
| TMPRSS4 | CAGGTCCAACTGGTCCAGTCTGGCGCCGAAGT | GACATTCAGATGACACAGAGTCCCAGTTCCTT |
| Ab9 | GAAAAAACCTGGAGCGTCCGTGAAGGTGTCCT | GTCGGCTTCAGTCGGTGATCGGGTGACTATAA |
| | GCAAGGCCTCCGGCTACACCTTCACCCGTTAT | CCTGTCAGGCCTCCCAGGATATTTCTAGGTTC |
| | TACATGCATTGGGTTCGCCAGGCACCCGGGCA | TTGCATTGGTATCAGCAAAAGCCTGGGAAAGC |
| | AGGGCTGGAATGGATGGGGTGGATTAATCCGA | ACCCAAGCTGCTGATCTACGGTGCAAGCAACC |
| | ACTCCGGCGGTACAAATTATGCTCAGAAATTT | TGAAATCTGGGGTGCCATCTAGATTCAGCGGC |
| | CAGGGAAGGGTGACGATGACTCGGGATACTAG | AGCGGGAGCGGGACCGACTTTACACTTACCAT |
| | CACAAGTACAGTTTACATGGAGCTCTCGAGCC | CTCAAGTTTACAACCAGAGGACTTCGCCACTT |
| | TACGATCAGAGGACACCGCCGTGTACTATTGT | ATTACTGCCAGCAGAGCTACTCTACACCTCCC |
| | GCTAGAGGCGGCTCATGGGGCAGCGGGCCACT | ACCTTTGGCGGTGGCACCAAGGTTGAAATCAA |
| | CGGCTATTGGGGACAAGGGACGCTTGTCACTG | G (SEQ ID NO: 600) |
| | TATCCTCC (SEQ ID NO: 599) | |
| TMPRSS4 | CAAGTGCAGCTTGTTCAGTCTGGAGCCGAGGT | GAAATTGTGATGACTCAGTCTCCTGCTACTCT |
| Ab10 | TAAAAAGCCCGGCGCGTCCGTTAAAGTGTCCT | GAGTGTATCACCTGGGGAAAGGGCAACTCTGA |
| | GCAAAGCCTCTGGCTACACCTTCACGAGCTAC | GTTGTAGAGCCTCGCAGAGCGTGTCCTCCTAT |
| | TACATGCATTGGGTCCGGCAGGCACCGGGCCA | CTGGCGTGGTACCAGCAGAAGCCCGGGCAGGC |
| | AGGGTTGGAGTGGATGGGGATTATTAATCCAT | ACCAAGGCTATTGATCTACGGAGCCAGCACTA |
| | CTGGTGCCGGGACCACATATGGGCACAACTTT | GAGCCACCGGGATACCCGCCCGTTTTTCTGGC |
| | CAGGGAAGAGTCACAATGACTCGAGACACCTC | AGCGGGTCAGGCACTGAGTTCACCTTAACCAT |
| | GACAAGCACCGTCTACATGGAACTCTCTAGCC | CTCCTCCCTGCAAAGTGAGGACTTCGCTGTCT |
| | TGCGCTCAGAGGACACGGCTGTGTACTATTGT | ATTACTGCCAGCAATATGGTAGCAGCCCAGGC |
| | GCTCGCGGCCCTAGGGATACCGCAATGGTGCG | ACGTTTGGCCAGGGTACAAAGCTCGAAATCAA |
| | GTTCGATTATTGGGGTCAGGGAACACTTGTAA | G (SEQ ID NO: 602) |
| | CAGTGTCCAGT (SEQ ID NO: 601) | |
| TMPRSS4 | CAGGTCCAGCTTGTCCAGTCTGGGGCGGAAGT | GACATTCAGATGACACAGAGTCCCTCATCACT |
| Ab11 | TAAAAAGCCTGGGGCTAGTGTAAAGGTGTCCT | GTCAGCCTCCGTTGGTGATAGAGTGACAATTA |
| | GCAAGGCCTCCGGCGGCACATTTAGCAATTAC | CGTGCCGTGCCTCTCAGTCCATCAACAACTAC |
| | GCAATCAGTTGGGTGCGGCAGGCTCCAGGTCA | TTAAACTGGTATCAGCAAAAACCGGGCAAAGC |
| | AGGGTTGGAGTGGGTGGGCCGGATTAATCCCA | ACCAAAACTGCTGATCTACGCGGCAAGCAGTC |
| | ACTCCGGCGGCACTAATTATGCTCAGAAGTTT | TCCAGTCTGGCGTGCCTAGCCGCTTTAGCGGG |
| | CAAGGGCGCGTTACCATGACCAGGGATACCAG | TCTGGTAGCGGCACTGATTTCACACTGACAAT |
| | CACTAGCACCGTCTACATGGAACTAAGCTCCC | AAGTAGCCTTCAGCCCGAGGACTTCGCCACCT |
| | TGAGGTCTGAGGACACTGCTGTCTACTATTGT | ATTACTGTCAACAGTCGTATTCCACAAAATGG |
| | GCCAGAGGACGATACTCCTCTTCGTCCTGGGG | ACTTTCGGGCAAGGGACGAAAGTGGAAATCAA |
| | TCAGGGAACCCTCGTGACCGTATCATCT (SEQ | G (SEQ ID NO: 604) |
| | ID NO: 603) | |
| TMPRSS4 | CAGGTTCAGCTTGTCCAGTCTGGGGCAGAAGT | GACATTCAGATGACTCAAAGCCCAAGCTCACT |
| Ab12 | CAAGAAGCCTGGCGCCTCCGTGAAAGTGTCGT | GTCAGCTAGTGTGGGCGATAGAGTGACGATTA |
| | GCAAGGCCTCCGGCTATACCTTCTCAAACTAC | CATGTAGGGCCTCCCAAGGGATCTCGCGCTGG |
| | TACATCCACTGGGTTCGACAGGCGCCCGGGCA | CTCGCGTGGTACCAGCAGAAGCCTGGGAAGGC |
| | GGGCTTGGAATGGATGGGCTGGATTAATCCGA | ACCCAAACTGCTGATCTACGGCGCCAGCAACC |
| | ACTCCGGTGATACAAATTATGCACAAAAATTC | TCCAGACTGGGGTGCCCTCTCGTTTTTCTGGA |
| | CAAGGGCGGGTAACAATGACCCGGGATACCAG | TCTGGTAGCGGCACTGACTTCACCCTGACAAT |
| | TACATCTACGGTCTACATGGAGCTAAGCAGCC | ATCCAGCTTACAGCCCGAGGACTTTGCCACCT |
| | TGCGCAGTGAAGATACCGCTGTGTACTATTGC | ATTATTGTCAACAGAGTTATAGCACTCCTCCA |
| | GCTAGAGGCATGACCTGGAGGACCTCTGCTGC | ACTTTTGGGCCAGGGACGAAAGTCGACATCAA |
| | CACATACTGGGGTCAGGGAACACTTGTTACTG | G (SEQ ID NO: 606) |
| | TGTCCTCC (SEQ ID NO: 605) | |
| TMPRSS4 | CAGGTTCAGTTGGTTCAGTCTGGGGCAGAAGT | GAGATTGTCATGACACAGTCTCCCGCTACTCT |
| Ab13 | TAAAAAGCCAGGCGCGAGCGTGAAAGTGTCTT | GAGTGTGTCGCCCGGCGAAAGGGCAACCCTGA |
| | GTAAGGCCTCCGGCTATACCTTCACCAATTAC | GTTGCAGAGCATCCCAGTCGGTGAACGGGAAT |
| | TACATGCATTGGGTCCGGCAGGCTCCTGGGCA | TATCTCGCCTGGTATCAGCAAAAACCGGGCCA |
| | AGGGTTGGAGTGGATGGGTTGGATAAGCGCCT | GGCTCCCAGGCTGTTAATCTACGGAGTCTCCT |
| | ACAACGGCAACACCAATTACGCTCAGAAACTG | CCCGGGCCAGCGGGATTCCTGCTCGTTTCAGC |
| | CAAGGGCGCGTGACGATGACAAGAGACACTTC | GGCTCCGGATCAGGCACTGAGTTCACACTAAC |
| | AACTTCAACCGTCTATATGGAACTGAGTAGCC | CATAAGTAGCCTTCAGTCTGAAGATTTTGCCG |
| | TCCGATCTGAGGACACCGCCGTGTACTATTGT | TGTACTATTGTCAACAGTATGGAAGCTCTCCA |
| | GCAACAGCCAGCGGTTGGGGACACAGTAACTC | TACACGTTTGGGCAGGGAACAAAGGTTGAGAT |
| | CGCGGGCTACTGGGGTCAAGGGACTCTTGTGA | CAAG (SEQ ID NO: 608) |
| | CAGTCTCTTCA (SEQ ID NO: 607) | |
| TMPRSS4 | GAAGTCCAACTGCTCGAGAGCGGCGGTGGGCT | GACATCCAAATGACACAGTCACCTTCATCCTT |
| Ab14 | GGTTCAGCCAGGCGGTAGTCTCCGGCTGTCCT | GTCCGCCTCTGTCGGCGATAGAGTGACGATTA |
| | GTGCTGCGAGTGGGTTCACCTTTTCCAATCAC | CATGTAGGGCAAGTCAGAGGATTAGCACTTAT |
| | TATATGAGCTGGGTGCGGCAGGCTCCGGGAAA | CTGAATTGGTACCAGCAGAAGCCCGGAAAAGC |
| | AGGCCTGGAGTGGGTATCAGCCATCTCCGGGT | ACCCAAACTGCTGATCTACTCCGCATCGACCC |
| | CTGGCGGCAGCACTTATTACGCCGATAGCGTT | TCCAGGCTGGGGTGCCATCTCGTTTTAGCGGG |
| | AAGGGTCGCTTCACCATTAGCCGGGACAACTC | TCTGGCTCAGGCACCGATTTCACGCTTACAAT |
| | CAAGAACACACTGTACTTGCAGATGAACTCAT | AAGTAGCCTCCAGCCCGAGGACTTTGCCACTT |
| | TACGCGCTGGGGATACCGCCGTGTACTATTGC | ATTACTGCCAACAGGCCTACTCTCTTCCTTGG |
| | GCGAGGGACCGATACAGATGGGGCAGAGGCTA | ACCTTCGGGCAGGGAACTAAGTTAGAAATCAA |
| | TTTTCAGCATTGGGGACAAGGGACTCTAGTAA | G (SEQ ID NO: 610) |
| | CAGTGTCCTCG (SEQ ID NO: 609) | |
| TMPRSS4 | CAGGTTCAGCTGGTCCAGTCGGGCGCCGAAGT | GACATACAGATGACCCAGTCGCCAAGCTCCTT |
| Ab15 | GAAGAAGCCTGGGGCGAGTGTTAAAGTGTCCT | GAGTGCTTCCGTCGGAGATAGAGTGACTATAA |
| | GCAAGGCCTCTGGCTACACTTTCACTCGTTAT | CCTGCCGAGCCTCTCAGTCAATCAATACCTGG |
| | TACATGCATTGGGTTCGGCAGGCACCTGGGCA | CTCGCCTGGTATCAGCAAAAGCCCGGCAAAGC |
| | AGGGCTGGAATGGATGGGGTGGATTAACCCGA | TCCCAAACTGCTGATCTACGCAGCAAGCAGTC |
| | ACTCCGGAGTGACCAATTTTGCTCAGAAGTTC | TCCAGAGCGGCGTGCCCTCACGCTTTTCTGGG |
| | CAAGGGCGCGTAACTATGACACGGGACACCAG | TCTGGGAGTGGTACAGATTTCACCCTTACAAT |
| | TACATCAACCGTCTACATGGAGCTGTCCAGCC | TAGCAGCTTGCAGCCAGAGGATTTTGCCACGT |
| | TAAGGAGCGAAGACACTGCTGTGTACTATTGT | ATTATTGTCAACAGGCCATCTCCTTTCCCTTA |
| | GCGAGGGTCAGAATTGGCTGGCTCCAGTCACC | ACCTTCGGCGGCGGGACAAAAGTGGAGATCAA |
| | TCCACTGTACTGGGGACAAGGAACGCTTGTGA | G (SEQ ID NO: 612) |
| | CAGTATCCAGC (SEQ ID NO: 611) | |
| TMPRSS4 | CAGGTCCAGCTCGTACAGTCCGGCGCGGAGGT | GACATTCAGATGACCCAGTCTCCATCATCACT |
| Ab16 | TAAAAAGCCTGGTGCAAGTGTAAAAGTTTCCT | GTCAGCTTCTGTGGGCGATAGAGTGACAATAA |
| | GCAAGGCCTCCGGTTACACTTTCTCACGGCAC | CATGCAGGGCCTCCCAGTCGATCAATCGCTGG |
| | TACATGCATTGGGTCCGGCAGGCACCCGGGCA | CTCGCGTGGTATCAACAGAAGCCCGGGAAAGC |
| | GGGCTTGGAATGGATGGGGCGCATTAATCCGA | ACCAAAGCTGCTGATTTACGGCGCCAGCAACC |
| | ACTCCGGCGGTACTAATTACGCTCAGAAATTT | TACAGAGCGGCGTGCCTAGTCGTTTTAGCGGT |
| | CAAGGGCGAGTTACCATGACAAGGGATACCTC | TCAGGGTCTGGAACCGACTTCACGTTGACAAT |
| | TACTTCCACCGTGTATATGGAACTGAGTAGCC | AAGCAGTTTACAGCCCGAGGACTTTGCCACCT |
| | TTAGGAGCGAGGACACCGCTGTGTACTATTGT | ATTATTGTCAACAGGCTAACTCCTTCCCATAC |
| | GCCAGAAGCATCTACGGCGATTATTGGTTTGA | ACTTTCGGGCAAGGCACTAAGCTGGAAATCAA |
| | TCCTTGGGGACAAGGGACACTTGTCACAGTGT | G (SEQ ID NO: 614) |
| | CTAGT (SEQ ID NO: 613) | |
| TMPRSS4 | CAAGTGCAGCTTGTTCAGTCCGGCGCTGAAGT | GACATTCAGATGACCCAGTCTCCAAGCAGCCT |
| Ab17 | GAAAAAACCCGGCGCATCAGTTAAAGTGTCCT | GAGTGCCTCGGTTGGGGACAGAGTGACTATTA |
| | GCAAGGCCTCCGGCTATACTTTCACATCGTAT | CCTGTCGCGCTAGTCAGGGAATCTCCTCCTAC |
| | TACATCCACTGGGTCCGGCAGGCTCCTGGGCA | CTGAACTGGTATCAACAGAAGCCCGGGAAGGC |
| | GGGACTAGAGTGGATGGGGTGGATGAGCCCTA | ACCCAAACTCTTGATTTACGCGGCGAGTAGAC |
| | ATAGCGGCGATACCGGTTACGCCCAAAAGTTT | TCCAAAGCGGAGTGCCGAGCAGGTTTTCCGGG |
| | CAGGGCCGCGTAACCATGACCAGGGACACATC | AGCGGGTCTGGCACCGATTTCACACTGACAAT |
| | TACATCCACCGTCTACATGGAGCTTAGCTCTT | AAGTAGTTTACAGCCAGAGGACTTCGCAACTT |
| | TGCGATCCGAAGATACGGCTGTGTACTATTGC | ACTATTGTCAACAGTCTTATAGAAGCCCTCCA |
| | GCCCGGCTCGTCCGTGGCGGGTTTGATTACTG | ACTTTCGGGCAGGGAACTAAGCTGGAAATCAA |
| | GGGTCAGGGAACGCTGGTAACAGTGTCAAGT | G (SEQ ID NO: 616) |
| | (SEQ ID NO: 615) | |
| TMPRSS4 | CAGGTCCAGCTCGTACAGAGTGGCGCCGAAGT | GACATTGTGATGACACAGAGTCCGGACAGCCT |
| Ab18 | CAAGAAGCCTGGAGCTTCCGTCAAAGTGTCCT | TGCTGTGTCTCTCGGTGAACGCGCGACCATCA |
| | GCAAAGCCAGCGGCTATACTTTCACGTCCTCA | ACTGCAAGTCCTCCCAGTCGGTGCTGTACTCA |
| | GGAATTAATTGGGTTCGGCAGGCACCCGGGCA | AGCAACAACAAGAATTACCTGGCCTGGTATCA |
| | GGGCTTGGAGTGGATGGGGTGGATTAATCCCA | ACAGAAACCGGGCCAGCCTCCAAAGCTGCTGA |
| | ACTCCGGCGGCGCAAAATATGCCCAACGCTTC | TCTACTGGGCTAGTACCAGGGAGTCTGGGGTG |
| | CAGGGACGGGTTACTATGACCCGAGATACCAG | CCTGATCGTTTCTCCGGTAGCGGCTCCGGGAC |
| | CACCTCTACTGTCTACATGGAGCTGAGCAGCC | AGACTTCACCCTAACTATAAGCAGTTTACAAG |
| | TTAGGAGTGAAGATACCGCTGTGTACTATTGC | CAGAGGACGTGGCCGTATATCACTGTCAACAG |
| | GCCAGGGCGAGAGGGTATTCTGGCTCGAAAAG | TACTACAATACACCATTTACGTTTGGACCAGG |
| | AGATTTTCAGCATTGGGGACAAGGGACATTGG | GACAAAAGTGGACATCAAG (SEQ ID NO: 618) |
| | TTACAGTCTCATCA (SEQ ID NO: 617) | |
| TMPRSS4 | CAAGTGCAGTTGGTTCAGTCTGGAGCCGAAGT | GATATTCAGATGACCCAGTCGCCAAGTTCCTT |
| Ab19 | GAAGAAGCCTGGGGCGAGCGTCAAAGTGTCCT | GTCCGCTTCCGTAGGCGACCGCGTGACTATAA |
| | GCAAGGCCTCCGGCTACACTTTTAACCGGAAA | CTTGCAGGGCCTCTCAAAGTATCTCGAGATAC |
| | TTCATGCATTGGGTTCGGCAGGCACCCGGTCA | CTGAATTGGTACCAGCAGAAGCCAGGGAAAGC |
| | AGGGCTGGAATGGATGGGCTGGATGAACCCGA | TCCAAAACTGCTGATCTACGGAGCGAGCAATC |
| | ACAACGGTGCAACTAATTATGCACAGAAGTTC | TCCAGAGCGGCGTGCCGAGTCGTTTTAGCGGC |
| | CAGGGCCGCGTGACAATGACACGAGATACCAG | AGCGGGTCTGGGACAGACTTCACGCTGACAAT |
| | CACTTCAACCGTCTACATGGAGCTCTCTTCCC | AAGTAGCTTACAGCCCGAGGATTTTGCTACGT |
| | TAAGGAGCGAAGATACCGCCGTCTACTATTGT | ATTACTGTCAACAGAGTTACTCCACACCTCCC |
| | GCTAGAGGAAGAGGCTATTATGGTTCTGGTTC | ACCTTCGGCCCTGGGACGAAAGTGGACATCAA |
| | ATACTATGGGGACTATTGGGGACAAGGGACCC | G (SEQ ID NO: 620) |
| | TTGTAACAGTCTCCTCT (SEQ ID NO: 619) | |
| TMPRSS4 | CAAGTGCAGCTGGTGCAGAGTGGCGCGGAGGT | GACATTCAGATGACCCAGTCGCCCTCTTCACT |
| Ab20 | CAAAAAGCCTGGGGCGTCCGTGAAAGTGTCCT | GTCCGCCTCAGTTGGGGACAGAGTAACCATTA |
| | GCAAGGCCTCCGGATACACCTTCACCCGATAT | CCTGTAGGGCCTCTCAGAATATCGCCACATAC |
| | TACATGCATTGGGTTCGGCAGGCACCAGGCCA | CTGAGCTGGTATCAGCAAAAGCCTGGCAAAGC |
| | AGGGCTCGAATGGATGGGGTGGATGAATCCCA | TCCAAAGCTGCTGATCTACGGTGCAAGCGCCC |
| | ACTCCGGGAATGCAGGCTATGCTCAGAAACTG | TCCGGTCTGGAGTCCCTAGCCGTTTTTCTGGG |
| | CAAGGCCGCGTGACTATGACTCGCGACACCTC | TCCGGGAGCGGGACAGATTTCACTCTCACAAT |
| | CACCTCAACTGTCTACATGGAGCTAAGTAGCT | ATCTAGCTTACAGCCGGAAGATTTCGCTACTT |
| | TGAGGTCTGAGGACACCGCTGTGTATTACTGT | ATTACTGCCTTCAGCACAACACATATCCCTTG |
| | GCCAGAGGCTACAACTGGTTTGATCCATGGGG | ACATTTGGCGGAGGCACGAAGGTTGAAATCAA |
| | TCAGGGAACACTTGTAACAGTGTCAAGT (SEQ | G (SEQ ID NO: 622) |
| | ID NO: 621) | |
| TMPRSS4 | CAGGTTCAACTGGTCCAGTCAGGCGCTGAAGT | GACATTCAGATGACTCAGAGTCCAAGCAGTTT |
| Ab21 | GAAGAAGCCTGGCGCCTCTGTGAAAGTGTCCT | GAGTGCTTCTGTGGGCGACAGAGTAACTATTA |
| | GCAAAGCCTCCGGTTATAGTTTCTCTGGCTAC | CATGCAGGGCCTCCCAGTCAATCTCTCGCTAT |
| | TATCTGCATTGGGTTAGGCAGGCACCTGGGCA | CTCAACTGGTATCAGCAAAAACCCGGCAAGGC |
| | GGGCCTGGAATGGATGGGGTGGATGAATCCCG | ACCGAAGTTACTGATCTACGCAGCGAGCACGC |
| | ATTCTGGGAATACTGGCTACGCGCAAAACTTT | TACAGAGCGGAGTGCCATCAAGATTTAGCGGC |
| | CAGGGACGGGTTACAATGACCCGAGATACTTC | AGCGGGTCCGGGACCGATTTCACCCTTACGAT |
| | CACCTCAACCGTCTACATGGAACTCTCCTCTC | AAGCAGCTTGCAACCTGAGGACTTCGCCACCT |
| | TGCGGAGCGAGGACACCGCCGTGTACTATTGT | ACTATTGTCAACAGTCTTACTCCACACCCGTG |
| | GCTCGCCTGCACCGTGGCGGGCACGATTACTG | ACATTTGGGCAGGGAACACGGCTCGAGATCAA |
| | GGGTCAGGGAACACTTGTCACTGTATCGAGT | G (SEQ ID NO: 624) |
| | (SEQ ID NO: 623) | |
| TMPRSS4 | CAGGTTCAGCTGGTCCAGTCTGGCGCCGAAGT | GACATTCAGATGACTCAGAGTCCAAGTTCACT |
| Ab22 | TAAAAAACCTGGCGCGAGCGTGAAAGTGTCCT | GTCAGCTTCCGTGGGTGACAGAGTGACGATAA |
| | GCAAGGCCTCCGGTTACACATTCACTTCATAT | CATGTAGGGCCGGGCAGAACATCAAGCGATAC |
| | TACATGCATTGGGTGCGGCAGGCTCCTGGGCA | CTCAACTGGTACCAGCAGAAACCTGGGAAGGC |
| | AGGGTTGGAGTGGATGGGGCGGATTAATCCGC | ACCCAAACTGCTGATATATGCAGCGAGCAGCC |
| | ACTCTGGCGACGCCGATTTCGTCGATAAGTTT | TCCAGTCTGGGGTACCCTCTCGTTTCAGCGGC |
| | CAAGGGCGCGTGACTATGACCAGGGATACTAG | TCTGGGTCCGGCACCGATTTTACGCTCACAAT |
| | CACCAGCACCGTCTACATGGAGCTATCAAGTT | CAGTTCCCTGCAACCAGAGGACTTTGCTACAT |
| | TGCGCTCCGAAGATACCGCAGTGTACTATTGC | ACTATTGTCAACAGTCGTACAGCTCGCCCTTA |
| | GCCAGGGACAGAAGAGGATATGGCGGCAATAG | ACCTTCGGAGGCGGCACCAAAGTCGAAATCAA |
| | CCTTGACTATTGGGGACAAGGGACACTTGTAA | G (SEQ ID NO: 626) |
| | CTGTTAGTTCT (SEQ ID NO: 625) | |
| TMPRSS4 | CAGGTTCAGTTGGTACAATCAGGAGCAGAAGT | GAAATCGTGATGACTCAGAGTCCCGCCACACT |
| Ab23 | GAAAAAGCCTGGAGCCTCAGTGAAGGTGTCCT | GAGTGTCAGCCCGGGCGAGCGGGCAACACTGA |
| | GCAAAGCCTCCGGCTATACATTCACCCGGAAT | GTTGCAGAGCCTCCCAGTCTGTCGGCAACTAT |
| | TACCTCCATTGGGTTCGGCAGGCTCCTGGCCA | CTAGCTTGGTATCAGCAAAAGCCTGGGCAGGC |
| | GGGCCTGGAGTGGATGGGGATTATTAATCCCA | TCCAAGGCTGCTTATTTACGGCGCGAGCACTA |
| | GTGGCGGCAGCACCACTTACGCCCAGAAGTTC | GAGCAACTGGGATACCAGCGCGTTTTTCTGGG |
| | CAAGGCCGCGTAACAATGACCCGAGATACATC | TCTGGGTCTGGGACTGAGTTTACCCTTACCAT |
| | AACTTCCACCGTCTACATGGAACTCTCCAGCC | CAGCTCGTTACAGTCTGAGGACTTCGCAGTGT |
| | TGAGGAGCGAGGACACGGCTGTGTACTATTGT | ACTATTGTCAACAGTACCACTCTAGCCCACCA |
| | GCCCGCGGCAGGACATGGTTCAGATCAGGCAT | TATACGTTTGGGCAGGGTACGAAAGTCGAAAT |
| | GGATGTGTGGGGTCAAGGGACCACCGTTACAG | CAAG (SEQ ID NO: 628) |
| | TGTCCTCC (SEQ ID NO: 627) | |
| TMPRSS4 | CAAGTGCAGCTGGTCCAGTCAGGCGCGGAAGT | GACATTCAGATGACCCAGTCTCCCTCGTCCTT |
| Ab24 | CAAAAAATCTGGCGCGAGCGTTAAGGTGTCCT | GTCAGCCAGCGTGGGTGATCGCGTTACCATTA |
| | GCAAGGCCTCCGGTTACACCTTTACGTCCTAT | CCTGTAGGGCTAGTCAGAGTATCTCCTCCTGG |
| | TACATGCATTGGGTCCGGCAGGCACCTGGGCA | CTCGCTTGGTATCAGCAAAAGCCCGGCAAAGC |
| | GGGCCTGGAGTGGATGGGCGTTATAAATCCCT | TCCTAAGCTGCTGATCTACGCAGCCTCAACTT |
| | CTGGCGGCACTACAAGCTACGCCCAGAAGTTT | TGCAGTCCGGCGTACCGAGTCGTTTTTCTGGG |
| | CAAGGGCGGGTGACAATGACTCGAGAGACAAG | AGCGGGAGCGGGACCGACTTCACGCTAACTAT |
| | CACTAGCACCGTCTACATGGAACTCAGCTCGC | TAGTAGCTTACAGCCAGAGGACTTCGCCACTT |
| | TGCGCTCAGAAGATACCGCCGTGTATTACTGC | ATTATTGTCAACAGTCATACAACACGCCATAC |
| | GCAAGAGGAAGAGGGTGGCTGAGGTCTGCTCT | ACATTCGGGCAAGGCACAAAACTTGAGATCAA |
| | CGGCTATTGGGGTCAGGGTACACTTGTAACCG | G (SEQ ID NO: 630) |
| | TGTCCTCC (SEQ ID NO: 629) | |
| TMPRSS4 | CAGGTCCAGTTAGTGCAGTCTGGGGCGGAGGT | GAAATCGTGATGACACAGAGCCCTGCTACCCT |
| Ab25 | GAAAAAGCCAGGAGCGAGCGTGAAAGTGTCCT | GAGTGTTAGCCCTGGGGAAAGGGCTACCCTCT |
| | GCAAAGCCTCCGGCGGGCGATTTTCAACATAC | CGTGTAGAGCCTCACAATCTGTCTCCTCCAAT |
| | GCCCTGTCATGGGTTCGGCAAGCACCCGGCCA | TATCTCGCCTGGTATCAACAGAAGCCCGGGCA |
| | GGGCCTGGAGTGGATGGGGTGGATTAACCCGA | GGCTCCCAGGTTGCTTATCTACGGGATTTCAA |
| | ACTCCGGCGGCACAAATTACGCCCAGAAATTC | CTAGAGCATCGGGTATTCCCGCTAGATTTTCT |
| | CAGGGCCGCGTAACTATGACTCGAGACACCTC | GGTTCTGGCAGCGGGACCGAGTTTACCCTTAC |
| | AACTTCCACCGTCTACATGGAGCTAAGCTCTC | GATAAGTAGCCTCCAGAGCGAGGATTTCGCAG |
| | TGAGGAGTGAAGACACAGCAGTGTACTATTGC | TGTACTATTGTCAACAGCGCTCTAACTGGCCA |
| | GCCAAGTCCTTGTGGTGGAGTCCAAGCCACTA | CCTAGCATAACGTTCGGGCAGGGAACTCGTCT |
| | CTATTACTATGGGATGGATGTGTGGGGTCAAG | GGAAATCAAG (SEQ ID NO: 632) |
| | GCACCACAGTCACAGTTTCCAGC (SEQ ID | |
| | NO: 631) | |
| TMPRSS4 | GAGGTTCAACTGCTCGAGTCTGGCGGCGGGCT | GACATTCAGATGACACAGTCTCCTAGTTCACT |
| Ab26 | TGTTCAGCCCGGCGGTTCTTTGAGGTTGTCAT | GTCAGCCAGCGTCGGTGATAGAGTTACAATAA |
| | GTGCCGCGTCTGGGTTTACCTTCTCCTCCTAT | CTTGTAGGGCCTCGCAGAATGTAGGAAGCTGG |
| | GCCATGCATTGGGTGCGGCAGGCTCCTGGCAA | CTCGCATGGTATCAACAGAAGCCAGGGAAAGC |
| | GGGCCTGGAGTGGGTGGCTGTCATCTGGTACG | TCCCAAGCTGCTGATCTACGCAGCAAGCAGCC |
| | ACGGGAGCAGCAAATACTACGCCGATTCCGTG | TCCAGAGCGGCGTACCATCGAGGTTTTCCGGC |
| | AAAGGCCGCTTCACCATTTCTCGAGACAACTC | AGCGGAAGTGGCACCGACTTCACCTTAACTAT |
| | AAAGAATACGCTGTACCTCCAGATGAACTCCC | TAGATCTCTACAGCCGGAAGATTTTGCCACCT |
| | TGCGCGCAGAAGATACCGCTGTGTACTATTGC | ATTACTGCCAACAGAGTTATTCCACTCCCATC |
| | GCGCGTGGGGAAGTGCGGAGAGGGTTCCAGCA | ACGTTTGGGCAAGGCACTCGGTTAGAGATCAA |
| | CTGGGGTCAGGGAACACTTGTCACAGTGTCCT | G (SEQ ID NO: 634) |
| | CC (SEQ ID NO: 633) | |
| TMPRSS4 | CAGGTTCAGCTGGTCCAGTCTGGCGCCGAAGT | GACATTCAGATGACACAGAGCCCTTCTTCACT |
| Ab27 | GAAGAAGCCCGGCGCATCCGTCAAAGTGTCGT | GTCAGCTTCTGTGGGCGATAGAGTGACTATTA |
| | GCAAGGCCTCCGGATATACATTTTCCCGCTAC | CCTGTCGTGCCAGTCAGTCAATCAGCACTTGG |
| | TACATGCATTGGGTTCGGCAGGCTCCTGGCCA | CTCGCTTGGTATCAGCAAAAGCCAGGGAAAGC |
| | AGGGTTGGAATGGATGGGGTGGATGAATCCGA | GCCCAAACTGCTGATCTACGCAGCGAGTAGTC |
| | ACTCCGGCGATACTGGTTATGCACAGAAATTC | TCCAGTCTGGCGTGCCCTCTCGCTTTTCGGGC |
| | CAAGGGAGGGTAACAATGACCCGCGACACCAG | TCCGGGTCCGGCACCGATTTCACCCTTACAAT |
| | CACATCTACGGTCTACATGGAACTCTCCTCAC | TTCAAGCCTACAGCCAGAGGACTTTGCCACCT |
| | TGCGAAGCGAGGATACTGCCGTGTATTACTGC | ATTACTGTCAACAGTTAAGCTCCTACCCATTG |
| | GCTAAGGGAAGGGAGTGGCTGAGATCTCCTTT | ACATTCGGGCAAGGGACAAAAGTAGAGATCAA |
| | CGACTATTGGGGTCAAGGGACTCTTGTGACCG | G (SEQ ID NO: 636) |
| | TTAGCAGC (SEQ ID NO: 635) | |
| TMPRSS4 | CAGGTTCAACTGGTACAGAGCGGCGCCGAAGT | GACATCCAGATGACCCAGTCACCCTCTTCACT |
| Ab28 | GAAAAAACCCGGCGCGAGCGTGAAAGTGTCCT | GTCAGCCAGCGTAGGCGATAGAGTCACTATTA |
| | GCAAGGCCTCCGGCTACACCTTCACTGGATAT | CCTGTAGGGCTTCGCAAGGGATTGGAAACTAT |
| | TACATGCATTGGGTCCGGCAGGCTCCCGGGCA | TTGGCTTGGTATCAACAGAAGCCCGGGAAGGC |
| | GGGCCTGGAGTGGATGGGATGGATGAATCCAA | ACCTAAACTGCTGATCTACGCAGCAAGCAGCT |
| | ACTCCGGAAATACCGGTTACGCCCAGAAATTC | TAGAAAGCGGAGTGCCGAGTCGCTTTAGCGGG |
| | CAGGGACGAGTTACAATGACTCGTGATACATC | TCCGGGTCTGGCACAGATTTCACGCTTACAAT |
| | CACTAGCACCGTCTACATGGAACTCTCTTCTC | AAGTAGTTTGCAGCCAGAGGATTTTGCCACGT |
| | TACGCTCCGAGGACACCGCAGTGTACTATTGC | ACTATTGTCAACAGGGCTACCGGTTCCCACCT |
| | GCTAGGCTCAGAGCGAAGGGCGGCGGTTTTGA | ACCTTTGGGCCTGGGACAAAGGTGGACATCAA |
| | CTATTGGGGTCAGGGTACACTTGTTACTGTGT | G (SEQ ID NO: 638) |
| | CAAGT (SEQ ID NO: 637) | |
| TMPRSS4 | CAGGTTCAGCTCGTACAATCTGGGGCAGAAGT | GACATTGTCATGACGCAGAGTCCGTTGTCCCT |
| Ab29 | GAAAAAGCCCGGCGCCAGCGTTAAGGTGTCCT | GCCCGTGACACCTGGGGAACCAGCGTCCATAT |
| | GCAAAGCCAGCGGCTATACCTTTGCAAATTAC | CTTGTAGATCATCACAGTCCCTCCTCCATTCG |
| | AACATCCACTGGGTGCGGCAAGCACCTGGGCA | AACGGCTACAACTACTTAGATTGGTACTTGCA |
| | GGGCCTGGAGTGGATGGGGTGGATGAATCCAA | GAAGCCCGGGCAATCGCCTCAGCTCCTGATCT |
| | ACTCCGGCAATACCGGTTACGCTCAGAAATTT | ACTTAGGATCTAATAGGGCCTCTGGAGTGCCA |
| | CAAGGACGGGTGACCATGACCAGGGACACTAG | GATCGCTTCTCAGGCAGCGGGAGCGGCACAGA |
| | CACATCAACTGTGTACATGGAGCTAAGCAGTC | TTTCACGCTGAAAATTAGTCGTGTTGAGGCCG |
| | TGCGAAGCGAAGATACCGCTGTCTACTATTGC | AAGACGTAGGCGTCTACTATTGTATGCAGAGC |
| | GCTCGCCCACGATATTCATCTGGGTGGTATGG | ACTTATTGGCCTCCAACTTTCGGGCAGGGAAC |
| | GTGGTATTTTGACCTGTGGGGCAGAGGTACAC | AAAGCTTGAGATCAAG (SEQ ID NO: 640) |
| | TTGTAACAGTGTCCAGT (SEQ ID NO: 639) | |
| TMPRSS4 | CAGGTTCAACTGGTCCAGTCGGGTGCCGAGGT | GAAATTGTTATGACTCAGAGTCCTGCCACCCT |
| Ab30 | GAAAAAGCCCGGAGCGTCAGTGAAAGTGTCCT | GAGTGTAAGTCCTGGGGAGAGGGCAACTCTGA |
| | GCAAGGCTTCTGGCTATACATTCACGACTTAT | GTTGTAGAGCCTCCCAGTCTGTGGGCAGATAT |
| | TACATGCATTGGGTGCGGCAGGCACCAGGCCA | TTGGCCTGGTACCAGCAGAAGCCTGGGCAGGC |
| | AGGGCTAGAATGGATGGGGTGGATGAATCCGA | TCCCAGGCTCCTCATCTACGGCGCGTCCACCA |
| | ACTCCGGAAACACAGGCTACGCCCAGAAGTTC | GAGCAACCGGGATTCCAGCTCGTTTTTCAGGC |
| | CAAGGGCGGGTAACAATGACCCGAGATACCAG | AGCGGCTCTGGCACAGAGTTCACGTTGACTAT |
| | CACCAGCACGGTCTACATGGAGCTCTCCTCAC | AAGCAGCTTACAGTCTGAGGACTTCGCCGTCT |
| | TGCGCTCCGAAGATACCGCAGTGTACTATTGT | ATTACTGCCAGCACTATGACAGCTCACCCATG |
| | GCCCGCGCTAGGACCTGGCTGCTGTCTCCATT | TACACTTTTGGGCAGGGAACAAAACTTGAAAT |
| | TGACTATTGGGGACAAGGGACTCTTGTGACCG | CAAG (SEQ ID NO: 642) |
| | TTAGTAGC (SEQ ID NO: 641) | |
| TMPRSS4 | CAGGTCCAGCTGGTGCAGTCTGGGGCAGAAGT | GAAATAGTTATGACTCAGTCGCCTGCCACACT |
| Ab31 | GAAAAAACCCGGAAGCAGCGTAAAGGTGTCCT | GAGTGTCTCGCCCGGCGAAAGGGCTACACTGT |
| | GCAAGGCCTCCGGTTACACCTTCCGCGGGTCT | CTTGCAGAGCCTCCCAAAGCGTTAGATCATAC |
| | GGCATCTCCTGGGTCCGGCAAGCTCCTGGGCA | CTGGCATGGTATCAGCAAAAACCTGGCCAGGC |
| | AGGGTTGGAGTGGATGGGGATTATCTACCCAG | TCCAAGGCTGCTCATATACGGCGCCTCAACCA |
| | CTGACAGCGAGACACGCTACAGCCCGAGTTTT | GAGCCACCGGCATTCCAGCCCGTTTTTCAGGG |
| | CAGGGCCGAGTGACCATTACCGCGGATGAGTC | AGTGGTAGCGGGACCGAGTTCACTCTTACGAT |
| | TACTTCAACTGCCTATATGGAACTCTCTAGTC | CAGCTCCCTACAGAGCGAGGACTTCGCAGTGT |
| | TCCGGTCCGAGGACACTGCGGTGTACTATTGT | ATTACTGTCAACAGCACGGCAGTTTGCCCTTA |
| | GCAAGGGAGAGTTCCTCCTGGGATTATTTCGA | |
| | TTATTGGGGACAAGGAACACTTGTAACAGTGT | ACCTTTGGTCAAGGGACGAAAGTCGAAATCAA |
| | CAAGC (SEQ ID NO: 643) | G (SEQ ID NO: 644) |
| TMPRSS4 | CAGGTTCAGTTGGTGCAGAGCGGCGCGGAGGT | GACATTCAGATGACCCAATCTCCATCTTCACT |
| Ab32 | CAAAAAGCCTGGGAGCAGCGTAAAAGTGTCCT | GTCAGCCTCAGTTGGGGATAGAGTCACCATTA |
| | GTAAGGCCTCCGGCGGCACATTCTCCTCATAC | CATGCAGGGCCTCTCAAAGCATCTCAACCTAC |
| | GCTATATCCTGGGTCCGGCAGGCTCCTGGGCA | CTGAACTGGTATCAGCAAAAACCCGGGAAAGC |
| | GGGCTTGGAATGGATGGGTCGGATTAATCCTA | ACCCAAACTGCTCATCTACGCTGCCTCCTCCC |
| | GCGGCGGGAGCACTTCTTATGCCCAAAAGTTT | TCCAGCGCGGCGTGCCGAGTCGTTTCTCTGGG |
| | CAAGGGCGCGTGACCATGACTCGAGATACCAG | TCAGGGAGCGGTACAGACTTTACGCTGACTAT |
| | TACAAGCACCGTCTACATGGAACTTAGTTCAC | CTCCTCCCTACAGCCAGAGGATTTTGCAACTT |
| | TGAGGTCTGAGGACACCGCAGTGTACTATTGT | ATTACTGCCAGCAGTCTTATACTACACCCTTA |
| | GCTAGAGGCCGATACAGTTCGTCGTCTTGGGG | ACCTTCGGCGGCGGCACAAAGGTTGAAATCAA |
| | TCAGGGTACGCTTGTGACTGTGTCCTCC (SEQ | G (SEQ ID NO: 646) |
| | ID NO: 645) | |
| TMPRSS4 | CAGGTCCAGCTTGTCCAGAGCGGCGCCGAAGT | GACATTCAAATGACCCAGTCACCCAGCTCATT |
| Ab33 | TAAGAAGCCTGGGGCGTCCGTAAAAGTGTCCT | GTCCGCCTCTGTAGGCGATAGAGTGACTATTA |
| | GCAAGGCCTCCGGATATACTTTTTCTAACTAC | CTTGTAGGGCTTCTCAGTACATCTCTCGCTGG |
| | TACATGCATTGGGTGCGACAGGCACCCGGCCA | TTGGCTTGGTATCAGCAAAAGCCAGGGAAAGC |
| | GGGTCTGGAGTGGGTGGGCTGGATGAACCCGA | ACCAAAACTCCTGATCTATGGGAGTTCTACCC |
| | AAAGCGGGAATACTGGTTATGCACAGAAATTT | TACAATCCGGCGTTCCATCTCGTTTCTCCGGG |
| | CAAGGGCGGGTGACAATGACCAGGGACACGAG | AGTGGCAGCGGAACCGATTTCACCCTCACGAT |
| | CACGAGCACAGTTTACATGGAACTCAGTTCAC | AAGCAGCTTACAGCCCGAGGACTTCGCTACAT |
| | TGCGCTCGGAGGATACCGCCGTCTACTATTGT | ATTACTGCCAACAGTACTACAGTACACCTTTC |
| | GCCAGAGGCCGGACCTGGATTCAGTCCTCGCT | ACCTTTGGCCCTGGGACAAAGCTGGAAATCAA |
| | GGGTTATTGGGGTCAGGGAACTCTTGTGACAG | G (SEQ ID NO: 648) |
| | TGTCCAGT (SEQ ID NO: 647) | |
| TMPRSS4 | CAGGTCCAGCTCGTCCAGAGCGGCGCCGAAGT | GACATTCAGATGACTCAGAGTCCCAGCTCACT |
| Ab34 | GAAAAAACCCGGAGCTTCAGTGAAAGTTTCCT | GTCAGCTTCTGTGGGCGACAGAGTGACCATTA |
| | GCAAAGCCTCCGGTTATACCTTCACTGGTTAC | CATGCCGCGCAAGTCAAGGGATCTCCTCCTGG |
| | TACATTCACTGGGTCCGGCAGGCTCCAGGCCA | TTGGCGTGGTATCAACAGAAGCCCGGCAAGGC |
| | AGGGTTGGAGTGGATGGGGTGGATGAATCCAC | ACCTAAGCTGCTGATCTACGCAGCCAGCACCC |
| | ATAGTGGCAACACCGGATACGCCCAGAAATTC | TACAGACTGGGGTGCCTTCTAGGTTTAGCGGT |
| | CAAGGGCGGGTTACAATGACACGTGATACCTC | AGTGGCAGCGGGACTGACTTCACGCTTACGAT |
| | TACTTCTACTGTGTATATGGAACTCAGCTCGC | CAGCTCCTTACAGCCGGAAGATTTTGCCACCT |
| | TGCGCTCTGAGGATACCGCTGTGTACTATTGC | ACTATTGTCAACAGTCCAAATCCATACCTATA |
| | GCCAGGGAGGGCGGTCGATACAGTTCTGGCAG | ACCTTTGGCGGCGGGACAAAGGTTGAGATCAA |
| | ACTGGGGTATTGGGGACAAGGGACACTTGTCA | G (SEQ ID NO: 650) |
| | CAGTATCCAGC (SEQ ID NO: 649) | |
| TMPRSS4 | CAAGTGCAACTGGTGCAGAGCGGCGCTGAGGT | GACATTCAGATGACACAGTCTCCAAGCTCCCT |
| Ab35 | TAAAAAGCCGGGCGCCAGCGTCAAAGTGTCCT | GTCCGCTTCAGTTGGAGATCGCGTTACAATTA |
| | GCAAGGCCTCCGGTTACACGTTCACCGGCTAC | CCTGTCAGGCCTCCCAGGACATCAGTAATTAC |
| | TACATGCATTGGGTCCGGCAGGCACCAGGCCA | CTGAATTGGTACCAGCAGAAACCCGGCAAAGC |
| | GGGCCTGGAATGGATGGGGAAAATCTCAGCCC | TCCCAAACTTCTCATATACAAGGCCTCCAGCC |
| | ACTCTGGGGAAACCAAGTATGCTCAGAACGTG | TCGAGAGCGGCGTGCCTAGTAGATTTTCTGGT |
| | CAAGGCCGAGTCACAATGACTAGGGACACGAG | TCTGGGAGTGGCACAGATTTCACCTTGACCAT |
| | CACTTCGACCGTCTACATGGAGCTGTCTAGCC | TTCAAGTTTGCAGCCTGAGGACTTCGCCACTT |
| | TACGGAGTGAAGATACCGCAGTGTATTACTGC | ATTACTGTCAACAGACTTATACGATACCCATC |
| | GCAAGGGCGAACTACTATGGGGATTATGTAAA | ACATTTGGGCAGGGAACTCGTTTAGAGATCAA |
| | CTACTATTATGGGATGGACGTCTGGGGACAAG | G (SEQ ID NO: 652) |
| | GGACTACCGTGACAGTGTCCTCT (SEQ ID | |
| | NO: 651) | |
| TMPRSS4 | GAAGTGCAGCTGCTCGAAAGCGGCGGCGGGCT | GACATTCAGATGACTCAGAGCCCATCTTCGCT |
| Ab36 | GGTGCAGCCTGGAGGTTCATTACGGTTGTCAT | GTCAGCTAGTGTTGGGGATAGAGTCACCATCA |
| | GCGCAGCGAGCGGCTTCACCTTCTCCTCCCGC | CCTGTAGGGCTTCTCAGTCAATCTCTACATGG |
| | GCTATGAGTTGGGTGCGACAGGCACCTGGGAA | CTCGCCTGGTATCAACAGAAGCCAGGCAAGGC |
| | AGGCCTGGAGTGGGTATCCAGGATTAATTATG | ACCCAAACTGCTGATCTACCGGGCCAGCAACC |
| | ATGGGTCTGCCACAACTTATGCCGACTCTGTT | TCCAGAGCGGCGTGCCGAGTCGTTTTAGCGGC |
| | AAGGGCCGATTTACCATATCCCGCGACAATTC | TCTGGAAGTGGTACTGATTTCACGCTCACCAT |
| | CAAAAACACGCTGTACTTGCAGATGAACTCGC | CTCCAGCCTACAGCCCGAGGACTTTGCCACAT |
| | TTAGAGCTGAGGACACCGCGGTGTACTATTGC | ATTACTGTCAACAAAGCTACAGCACACCCTTA |
| | GCCAGAGGCATAACTATTTTTGGGGTCTTCGA | ACTTTCGGCGGAGGCACAAAGGTCGAAATCAA |
| | TTACTGGGGACAAGGGACACTTGTAACCGTGT | G (SEQ ID NO: 654) |
| | CCTCC (SEQ ID NO: 653) | |
| TMPRSS4 | GAAGTCCAACTGCTCGAATCTGGCGGCGGCTT | GACATTCAGATGACTCAGAGCCCGAGTTCACT |
| Ab37 | GGTTCAGCCTGGCGGGAGCTTGCGGCTCTCAT | GTCAGCTTCGGTCGGAGATCGCGTGACCATAA |
| | GTGCTGCCAGCGGGTTCACCTTCTCCTCCTAT | CTTGCAGGGCTTCTCAGTCAATCAGCCGATAC |
| | GCCATGCATTGGGTTCGCCAGGCACCCGGCAA | CTGAACTGGTATCAACAGAAGCCTGGGAAAGC |
| | GGGCTTAGAGTGGGTGTCTTACATAAGTAGTA | GCCTAAGCTGCTGATCTACTCAGCATCCACTC |
| | GTGGCAGCACCGTGTACTACGCCGATTCTGTT | TCCAAAGCGGCGTGCCCTCCCGTTTTAGCGGC |
| | AAAGGGAGGTTCACCATCTCCCGGGATAATAG | TCTGGGTCTGGGACAGACTTCACACTAACAAT |
| | CAAGAATACGCTGTACCTCCAGATGAACTCCC | AAGTAGTTTACAGCCAGAGGACTTTGCTACCT |
| | TGAGAGCAGAGGACACCGCCGTGTACTATTGC | ATTATTGTCAACAGGCCCACTCTTTCCCGCCA |
| | GCCAGAGTATCTAACGTCACTCCCAGGAGCGG | TCCTTTGGGCAGGGTACAAAACTTGAAATTAA |
| | GTTTGGCTATTGGGGTCAGGGAACGCTTGTAA | G (SEQ ID NO: 656) |
| | CAGTGTCCAGT (SEQ ID NO: 655) | |
| TMPRSS4 | CAAGTGCAGCTGGCTCAGTCTGGGGCAGAAGT | GACATCCAGATGACCCAGTCACCATCTTCCCT |
| Ab38 | CAAAAAACCTGGCGCCTCTGTAAAAGTGTCCT | GTCAGCGAGCGTGGGCGACAGAGTAACTATTA |
| | GCAAGGCCTCCGGCTATACCTTCACCCGGCAC | CCTGTCAGGCTAGTCAAGACATTAGCAGATAC |
| | TACATCCAGTGGGTGCGGCAGGCACCTGGACA | CTCAACTGGTATCAGCAAAAGCCCGGGAAGGC |
| | AGGCTTAGAGTGGATGGGGTGGATTAATCCCA | ACCCAAGCTGCTCATCTACGGTGCCAGCAACC |
| | ACTCCGGCAATACGGGCTATGCCCAAAAATTC | TGCTGAGCGGCGTGCCTAGCAGGTTTTCTGGA |
| | CAAGGGCGCGTTACCATGACTCGAGATACGTC | AGTGGTTCTGGGACAGATTTCACTCTTACAAT |
| | CACATCTACGGTCTACATGGAACTGAGTTCCT | AAGTAGCCTACAGCCGGAGGATTTTGCCACCT |
| | TGCGCTCGGAGGACACAGCGGTGTATTACTGC | ACTATTGTCAACAAACTCATACTACTCCATAT |
| | GCTAGAGGCAGGCAATGGCTCCGTGGGGAATA | ACCTTTGGGCAAGGCACAAGGTTAGAGATCAA |
| | CTTCCAGCACTGGGGACAAGGGACCCTTGTCA | G (SEQ ID NO: 658) |
| | CAGTTTCCAGC (SEQ ID NO: 657) | |
| TMPRSS4 | CAAGTGCAGCTGGTACAGAGCGGCGCAGAGGT | GACATACAGATGACACAGTCGCCCTCATCACT |
| Ab39 | CAAAAAGCCTGGCGCGAGCGTCAAAGTGTCCT | GTCAGCCTCTGTTGGGGATAGAGTGACCATTA |
| | GCAAGGCCTCCGGCGGGAGTTTTAGCGGCTAT | CCTGTAGAGCTTCGCAAGGGATTAGGAACTGG |
| | GCTGTGTCCTGGGTTCGGCAGGCGCCTGGGCA | TTGGCCTGGTACCAGCAGAAGCCCGGCAAAGC |
| | GGGTTTGGAGTGGCTGGGCGTAATTAATCCAT | ACCAAAGCTGCTGATCTACAGAGCAAGCACGC |
| | CCGATTCTTGGACCGCCTTCGCTCAGAAATTT | TACAGAGCGGCGTGCCTAGCCGTTTTAGCGGG |
| | CAGGGCCGCGTTACTATGACTCGAGATACTTC | AGCGGTAGTGGCACAGACTTCACCCTTACCAT |
| | TACTTCTACCGTCTACATGGAACTTAGTAGTC | CTCCTCCTTACAACCCGAAGACTTCGCCACAT |
| | TCAGGTCTGAGGACACGGCAGTGTACTATTGC | ACTATTGTCAACAGAGTTATACAACTCCGTTT |
| | GCTAGGGAACGCGAGGATGATGCCTTCGACAT | ACATTCGGGCAGGGTACAAAGCTCGAGATCAA |
| | CTGGGGACAAGGGACCACGGTCACAGTGTCCT | G (SEQ ID NO: 660) |
| | CC (SEQ ID NO: 659) | |
| TMPRSS4 | CAAGTGCAGCTCGTTCAGTCTGGGGCAGAAGT | GACATTCAGATGACTCAGAGCCCTAGCTCGCT |
| Ab40 | GAAAAAACCTGGGGCGAGTGTCAAAGTGTCCT | GTCGGCTTCTGTAGGCGATAGAGTGACTATTA |
| | GCAAGGCCTCAGGCGGCACATTCTCAAGTTAT | CTTGCCGAGCTTCTCAGAGTATTTCCAGCTAC |
| | GCCATCTCCTGGGTTCGGCAGGCTCCAGGCCA | CTGAATTGGTACCAGCAGAAGCCCGGCAAAGC |
| | GGGCTTGGAATGGATGGGGATAATTAATCCGC | ACCCAAGCTGCTGATCTACGCGGCCTATAACC |
| | GTGGCGGGTCAACCAACTACGCTCAGAAGTTC | TTCAATCTGGGGTGCCATCAAGGTTCAGCGGC |
| | CAAGGGCGGGTAACAATGACCAGAGATACCAG | TCCGGGTCTGGCACAGACTTCACCCTTACAAT |
| | CACTAGCACCGTCTACATGGAGCTCAGCTCCC | CAGTAGCTTACAGCCCGAGGACTTTGCCACCT |
| | TACGCTCAGAAGATACCGCCGTGTACTATTGT | ACTATTGTCAACAGTCCTACTCAATCCCTTTT |
| | GCAAGGGAAGGTTCTAGCTGGTATTATGACGC | ACGTTTGGAGGTGGCACGAAGGTTGAGATCAA |
| | CTTTGATATATGGGGACAAGGAACTATGGTCA | G (SEQ ID NO: 662) |
| | CAGTGTCCTCC (SEQ ID NO: 661) | |
| TMPRSS4 | CAGGTCCAGCTCGTACAGAGTGGCGCAGAGGT | GACATTCAGATGACACAGAGCCCTAGCAGTCT |
| Ab41 | CAAAAAACCGGGCGCCAGCGTTAAAGTGTCCT | GTCAGCCAGCGTTGGCGACAGAGTGACCATCA |
| | GCAAAGCCTCCGGTGGCACATTCTCCTCCTAC | CCTGTAGGGCTTCTCAGTCAATAAGCCGGTGG |
| | GCTATCTCCTGGGTTCGGCAGGCTCCAGGTCA | TTGGCATGGTATCAACAGAAGCCCGGGAAGGC |
| | AGGGCTGGAATGGATGGGGTGGATGAATCCCA | ACCCAAGCTGCTGATCTACGCAGCGAGCACCC |
| | ACTCCGGCGATACACATTATGCCCAAAAGTTT | TCCAGACCGGCGTGCCATCGCGTTTTAGCGGG |
| | CAGGGCCGCGTAACCATGACTAGGGACACCTC | TCTGGCAGCGGCACAGACTTCACCCTTACAAT |
| | TACGTCAACTGTCTACATGGAGCTGTCATCAC | TAGTAGTTTACAGCCTGAGGACTTTGCGACTT |
| | TAAGATCTGAAGATACTGCCGTGTACTATTGT | ACTATTGCTTGCAGCACAGCTCTTATCCTTTC |
| | GCCCGAGAAGGGTCTAGTTGGTACTACGATGC | ACGTTCGGGCAAGGGACAAAGGTGGAGATCAA |
| | TTTTGATATTTGGGGACAAGGCACCCTTGTGA | G (SEQ ID NO: 664) |
| | CTGTGTCGTCC (SEQ ID NO: 663) | |
| TMPRSS4 | CAGGTCCAGCTCGTCCAGTCTGGGGCTGAAGT | GACATTCAGATGACCCAGTCGCCCTCGTCACT |
| Ab42 | GAAAAAACCCGGCGCCAGCGTTAAAGTGTCCT | GTCAGCGAGTGTGGGCGATAGAGTAACCATCA |
| | GCAAGGCCTCAGGCTATTCCTTCACCTCCCAC | CCTGTAGGGCATCTCAGAGCATCCGCAATTAC |
| | TACATGCATTGGGTTCGGCAGGCTCCTGGCCA | CTCAACTGGTACCAGCAGAAGCCAGGGAAAGC |
| | AGGGTTAGAATGGATGGGTTGGATGAACCCGA | ACCCAAGTTGCTGATTTACGAGGCCTCCAGAC |
| | ACTCCGGCAATACCGGTTATGCTCAGAAGTTC | TACAGTCTGGTGTGCCGAGCAGGTTTAGCGGT |
| | CAAGGGCGAGTCACAATGACTCGTGATACTAG | TCCGGGTCTGGCACAGACTTCACGCTCACAAT |
| | CACTTCTACAGTGTATATGGAGCTGAGTAGTC | AAGTAGCTTGCAGCCAGAGGACTTTGCCACCT |
| | TGCGCAGTGAAGATACTGCCGTATATTACTGT | ATTACTGCCAACAAAGCTATAGCGCTCCACCT |
| | GCACGGCTTGGGCAGCAGCTGGATTACTGGGG | ACATTTGGGCCTGGCACGAAGGTTGACATCAA |
| | ACAAGGGACTCTTGTGACCGTGTCCTCC (SEQ | G (SEQ ID NO: 666) |
| | ID NO: 665 | |
| TMPRSS4 | CAAGTGCAGCTCGTCCAGTCTGGGGCTGAAGT | GACATTCAGATGACACAGAGTCCCAGCTCCCT |
| Ab43 | GAAAAAACCGGGTGCCTCCGTCAAGGTGTCCT | GTCAGCCTCGGTTGGGGACCGCGTCACCATCA |
| | GCAAGGCCTCAGGCTACACTTTCATAGGGTAC | CCTGTCAGGCCTCACAAGATATTAGTAATTAC |
| | TACATGCATTGGGTACGGCAGGCACCTGGGCA | CTGAACTGGTATCAGCAAAAACCTGGCAAGGC |
| | AGGTCTAGAATGGATGGGGCGGATTAATCCCA | ACCAAAGCTGCTGATCTACGCGGCAAGCTCCT |
| | ACTCCGGCGAAACTAACTATGCTCAGAAGTTT | TACAGTCTGGGGTGCCCTCGCGTTTTAGCGGA |
| | CAGGGCCGAGTAACCATGACTAGGGACACCAG | AGTGGAAGTGGCACAGATTTCACGCTTACTAT |
| | CACCAGCACGGTCTACATGGAGCTGTCATCTT | CTCAAGCCTCCAGCCTGAGGATTTCGCCACTT |
| | TGAGGAGCGAGGACACCGCTGTGTACTATTGC | ATTATTGTCAACAGTCTTATAGCACACCAGTG |
| | GCTAGAGTTAGAGTGCGCGGCGTGATACACCC | ACATTCGGGCCAGGGACAAAAGTTGACATCAA |
| | TGGCTTTGATCCCTGGGGTCAGGGAACACTTG | G (SEQ ID NO: 668) |
| | TCACCGTGTCCTCC (SEQ ID NO: 667) | |
| TMPRSS4 | CAAGTGCAGTTAGTTCAGTCTGGTGCGGAGGT | GACATTCAGATGACTCAAAGCCCATCATCCCT |
| Ab44 | GAAAAAACCTGGGGCATCTGTGAAAGTGTCGT | GAGTGCCTCTGTGGGCGACCGGGTCACAATAA |
| | GCAAGGCCAGCGGCTATACCTTTCGGAACTAC | CCTGTCAGGCATCACAGGACATCAGCAATTAC |
| | TACATCCACTGGGTCCGCCAGGCTCCTGGTCA | CTGAATTGGTATCAGCAAAAGCCCGGGAAAGC |
| | GGGCCTAGAATGGATGGGCAGGATTAATCCAA | TCCCAAGCTGCTGATATACGCAGCGAGCAGCC |
| | ACTCAGGCGGCACAAACTACGCCCAGAAGTTT | TCCATAGCGGAGTACCTTCTCGATTCAGCGGG |
| | CAGGGACGTGTGACTATGACTAGGGATACCTC | TCCGGGTCCGGGACCGATTTCACTCTTACAAT |
| | CACCTCCACCGTCTACATGGAACTGAGTAGTC | CAGCTCATTGCAGCCCGAAGATTTTGCAACCT |
| | TCAGATCTGAGGATACTGCTGTGTACTATTGC | ATTATTGTCAAGAGTCCTCGTCCTTTCCGTAC |
| | GCCCGCGCCAGAATTGCTGTCGCCGTTTCCGG | ACGTTCGGGCCAGGGACGAAAGTTGACATCAA |
| | GTTCGGCTATTGGGGTCAGGGAACATTGGTAA | G (SEQ ID NO: 670) |
| | CAGTGTCAAGT (SEQ ID NO: 669) | |
| TMPRSS4 | CAGGTCCAGCTCGTCCAGAGTGGCGCGGAAGT | GACATTCAGATGACGCAGAGCCCATCTTCCCT |
| Ab45 | GAAAAAGCCTGGCGCCAGCGTAAAAGTGTCCT | ATCCGCTTCGGTTGGGGATAGAGTGACTATTA |
| | GCAAGGCCTCCGGCTATACCTTTTCACGGTGG | CCTGTCAGGCAACGCAGGACATACGCAATTAC |
| | TATATGCATTGGGTCCGGCAGGCACCAGGCCA | CTGAACTGGTACCAGCAGAAACCCGGCAAGGC |
| | AGGGCTGGAATGGATGGGTCGCATTAATCCCA | TCCGAAGCTGCTTATCTACGCAACATCATCAT |
| | ACTCCGGCGGCACTAACTATGCTCAGAAATTC | TACAAAGCGGAGTGCCATCTCGTTTTTCTGGG |
| | CAAGGGCGAGTAACTATGACTAGGGACACATC | AGTGGGTCAGGCACAGATTTCACCTTGACCAT |
| | TACCAGCACAGTTTACATGGAGCTGTCAAGTT | CAGCTCGCTTCAGCCCGAGGACTTCGCCACCT |
| | TGAGGTCTGAGGATACCGCCGTGTACTATTGC | ACTATTGTCAACAGAGTTACAGCCCTCCGTAC |
| | GCCAGAGTTGGCGGTTATGGGTGGTTTGATCC | ACGTTCGGGCAGGGAACAAAGCTCGAAATCAA |
| | TTGGGGACAAGGGACCCTGGTGACAGTGTCCT | G (SEQ ID NO: 672) |
| | CC (SEQ ID NO: 671) | |
| TMPRSS4 | CAAGTGCAGCTGGTACAGTCAGGCGCCGAAGT | GACATTCAGATGACACAGTCTCCAAGCAGTTT |
| Ab46 | CAAAAAGCCCGGAGCCTCGGTTAAAGTGTCCT | GAGCGCCAGTGTGGGCGACCGTGTAACTATCA |
| | GCAAGGCCTCTGGTTACACTTTTTCCCGGTAT | CGTGTAGAGCAAGTCAGTCAATCAGCACATGG |
| | TTCATGCATTGGGTGCGGCAGGCACCTGGCCA | CTCGCATGGTATCAACAGAAGCCCGGCAAAGC |
| | GGGTTTGGAGTGGATGGGGTGGATTAATCCCA | TCCAAAACTGCTGATCTACGCGGCGAGCAGTT |
| | ACTCCGGTGGCACCAACTACGCCCAAAAGTTT | TACAGTCCGGCGTGCCTTCGAGGTTTTCTGGG |
| | CAGGGAAGAGTGACTATGACCAGGGATACCTC | TCCGGGTCCGGGACCGATTTCACCCTTACAAT |
| | AACTTCCACCGTCTACATGGAACTCAGCTCTC | AAGCAGCCTACAGCCCGAGGATTTTGCTACGT |
| | TGCGCAGCGAGGACACCGCTGTGTACTATTGC | ACTATTGTCAACAGTCTTATGGGTTCCCGTGG |
| | GCTAGAGTCCGAATTGGGTGGCTCCAGTCACC | ACTTTCGGCCAGGGTACAAAGGTCGAAATCAA |
| | TCCACTGTACTGGGGACAAGGGACACTTGTTA | G (SEQ ID NO: 674) |
| | CAGTTTCCTCT (SEQ ID NO: 673) | |
| TMPRSS4 | CAGGTCCAGCTGGTGCAGAGCGGTGCAGAAGT | GACATTCAGATGACTCAGTCTCCAAGCTCGCT |
| Ab47 | CAAGAAGCCCGGTGCAAGCGTAAAAGTGTCCT | GTCAGCTAGTGTTGGCGACAGAGTAACTATCA |
| | GCAAGGCCTCCGGGTACACCTTCACCGGTTAT | CTTGTCGGGCCTCGCAGTCCATCTCCTCATAT |
| | TTCATGCATTGGGTCCGGCAGGCTCCTGGCCA | CTCAACTGGTATCAACAGAAACCGGGCAAGGC |
| | AGGGTTGGAATGGATGGGCTGGATGAATCCCA | ACCTAAGCTGCTCATTTACGCGGCCTCCTCCC |
| | ACTCCGGGAATACCGGCTACGCTCAAAAATTT | TACAGTCTGGGGTGCCTAGTCGTTTTAGCGGG |
| | CAAGGGCGCGTGACCATGACAAGGGATACAAG | TCTGGCTCTGGGACCGACTTCACTCTTACAAT |
| | CACCTCCACCGTGTATATGGAGCTCAGCTCTC | AAGTAGTTTGCAGCCAGAAGATTTCGCCACTT |
| | TGAGATCAGAGGATACAGCCGTTTATTACTGC | ACTATTGTCAACAGAGTTACTCTACGCCCTTA |
| | GTTCGAGGGAGGACTTGGATTCAGTCAAGCCT | ACCTTTGGCGGCGGCACAAAAGTGGAGATCAA |
| | GGGGTACTGGGGTCAGGGAACGCTTGTGACAG | G (SEQ ID NO: 676) |
| | TCTCATCT (SEQ ID NO: 675) | |
| TMPRSS4 | CAAGTGCAGCTGGTCCAGTCTGGGGCGGAAGT | GACATTCAGATGACCCAGTCACCAAGTTCCTT |
| Ab48 | GAAAAAGCCCGGAGCTAGTGTAAAGGTGTCCT | ATCCGCAAGCGTTGGGGATCGTGTTACAATTA |
| | GTAAAGCCAGCGGCTACACCTTCACCGGTTAT | CTTGCAGGGCCTCGCAAGGGATCTCTAATTAT |
| | TACCTGCATTGGGTCCGGCAGGCTCCTGGCCA | CTCGCTTGGTACCAGCAGAAACCTGGGAAAGC |
| | GGGCCTGGAGTGGATGGGCTGGATTTCCGCAT | ACCCAAGCTGCTGATCTACACTGCAAGCACAC |
| | ATAACGGAAACACAAATTACGCCCAGAACCTG | TTTTTCCAGGAGTGCCGTCAAGATTCTCTGGG |
| | CAAGGCCGCGTGACCATGACCAGGGACACAAG | TCCGGGAGTGGCACTGACTTCACCCTTACCAT |
| | CACTAGCACTGTCTACATGGAGTTGTCTAGCT | CTCCTCCCTCCAGCCTGAGGACTTTGCCACAT |
| | TGAGAAGCGAAGATACCGCTGTGTACTATTGC | ATTATTGTCAACAGAGTTACTCCATACCACTC |
| | GCCCGACACTCTTACTCGGGCTCATACTCAAC | ACGTTTGGCGGCGGAACAAAaGTtGAAATCAA |
| | GCTACCCTATTATGGGATGGATGTTTGGGGTC | G (SEQ ID NO: 678) |
| | AAGGGACAACGGTCACAGTATCCTCT (SEQ | |
| | ID NO: 677) | |
| TMPRSS4 | CAGGTCCAGTTGGTACAGAGCGGCGCCGAAGT | GACATTCAGATGACTCAATCTCCCTCGTCACT |
| Ab49 | GAAAAAGCCTGGGGCGTCCGTCAAAGTGTCTT | GTCAGCTAGTGTTGGGGATAGAGTGACTATTA |
| | GCAAGGCCTCCGGCTATACATTCACCGGGTAC | CCTGCCGAGCCAGTCAGTCAATATCTAACTGG |
| | TACATGCATTGGGTGCGGCAGGCACCTGGCCA | CTCGCATGGTACCAGCAGAAGCCAGGGAAGGC |
| | GGGTCTAGAATGGATGGGCCGGATCAATCCCA | TCCCAAACTGCTGATCTACGCCGCGAGCACCC |
| | ACTCCGGCGGCACAAACTATGCTCAGAAATTT | TTCAGAATGGCGTGCCGTCTAGATTTAGCGGT |
| | CAAGGTCGCGTCACCATGACCCGTGACACAAG | TCTGGGTCTGGGACCGACTTTACACTTACTAT |
| | TACGAGCACCGTCTACATGGAGCTGTCCTCCC | CAGTAGTTTACAACCAGAGGACTTTGCTACTT |
| | TCAGGAGCGAGGATACAGCCGTGTACTATTGT | ATTACTGTCAACAGAGCTACACCTTCCCTATT |
| | GCAAGGGAGCGCGCCGGCTATAGCAGCGGGCA | ACGTTCGGCCAGGGAACAAAAGTTGAAATCAA |
| | GTTCGATTATTGGGGACAAGGGACTCTGGTAA | G (SEQ ID NO: 680) |
| | CTGTGTCCTCC (SEQ ID NO: 679) | |
| TMPRSS4 | CAGGTCCAGCTGGTTCAGTCTGGCGCGGAAGT | GACATTCAGATGACCCAGTCGCCGTCCTCCCT |
| Ab50 | TAAAAAGCCAGGCGCCTCCGTCAAAGTGTCCT | ATCCGCCAGTGTCGGTGATCGCGTAACCATTA |
| | GCAAGGCCTCCGGCTATACTTTCACCGGTTAT | CGTGTAGGGCCTCTCAAGGGATCAGCAATTAC |
| | TACATGCATTGGGTGCGGCAGGCACCTGGGCA | CTCGCATGGTACCAGCAGAAACCCGGGAAGGC |
| | AGGGCTGGAATGGATGGGATGGATTAACCCGA | ACCCAAACTGCTGATCTACGCTACAAGCAGAC |
| | ACTCCGGAGGCACACACTATGCCCAAAAGTTT | TCCAGTCAGGCGTGCCCTCTCGCTTCTCTGGC |
| | CAGGGACGGGTTACAATGACTCGTGACACTTC | AGCGGGTCTGGCACCGATTTCACCCTTACAAT |
| | AACTAGCACCGTCTACATGGAGCTTAGTAGTT | AAGTAGCCTCCAGCCTGAGGATTTTGCTACGT |
| | TGAGGTCAGAGGACACCGCTGTGTATTACTGC | ATTATTGTCAACAGAGCTACAAGACTCCCTTA |
| | GCTAGAGTGCGAATCGGGTGGCTGCAGAGTCC | ACCTTTGGCGGCGGGACAAAAGTGGAAATCAA |
| | ACCACTGTACTGGGGACAAGGGACTTTGGTAA | G (SEQ ID NO: 682) |
| | CAGTGTCAAGC (SEQ ID NO: 681) | |
| TMPRSS4 | CAAGTGCAGCTGGTCCAGAGCGGTGCCGAAGT | GACATTCAGATGACGCAGAGTCCAAGCTCCTT |
| Ab51 | GAAAAAGCCTGGGGCGTCCGTGAAAGTGTCCT | GTCCGCTTCTGTGGGCGATAGAGTAACCATTA |
| | GTAAGGCCAGCGGATATACCTTCACCAACTAC | CTTGCAGGGCTTCACAGAGCATCTCTTCATAC |
| | TACATGCATTGGGTCCGGCAGGCTCCCGGCCA | CTGAACTGGTACCAGCAGAAACCCGGGAAGGC |
| | AGGGCTGGAATGGATGGGGTGGATTAATCCAA | ACCCAAACTTCTCATCTACGCTGCCTCCTCCC |
| | AATCTGGCGGCACTTCTTATGCACAGAAGTTC | TACAATCCGGCGTGCCGAGTCGTTTTTCAGGC |
| | CAGGGCCGCGTTACTATGACTAGGGATACAAG | TCGGGCTCTGGCACCGACTTCACACTCACGAT |
| | CACCAGCACTGTCTACATGGAACTGTCGAGTT | AAGTAGTTTACAGCCTGAGGACTTTGCCACCT |
| | TGAGAAGTGAGGATACAGCAGTGTATTACTGC | ACTATTGTCAACAGAGCTATAGCACACCTCTG |
| | GCCAGCGGGAAGCAATGGCTCGTAGGAGGTCG | ACCTTTGGCGGCGGGACAAAGGTTGAGATCAA |
| | ATTCGACTATTGGGGTCAGGGAACACTTGTCA | G (SEQ ID NO: 1000) |
| | CCGTTTCATCC (SEQ ID NO: 683) | |
| TMPRSS4 | CAGGTCCAGCTGGTTCAGTCTGGCGCAGAAGT | GACATTCAGATGACTCAGTCTCCAAGCTCCTT |
| Ab52 | GAAAAAGCCTGGGGCATCTGTAAAGGTGTCCT | GTCAGCGAGTGTTGGGGATAGAGTGACAATAA |
| | GCAAGGCCTCCGGATATACCTTCACTAGATAT | CTTGCAGGGCTAGTCAGGGTATTAGTCGGTGG |
| | TACATCCACTGGGTGCGGCAGGCACCTGGGCA | CTAGGCTGGTACCAGCAGAAACCCGGGAAGGC |
| | AGGGCTGGAATGGATGGGGTGGATGAATCCGA | TCCAAAACTGCTGATCTACGGCGCCAGCAACT |
| | ACTCCGGCAATACCGGGTTTGCCCAAAAACTG | TGCAGACTGGGGTGCCCTCGCGTTTCTCAGGC |
| | CAAGGGCGAGTAACAATGACCAGGGATACCAG | TCAGGCTCTGGGACTGACTTCACCCTTACCAT |
| | CACAAGCACGGTCTACATGGAGCTCAGCTCCC | TAGTAGCTTACAGCCCGAAGATTTTGCCACCT |
| | TCCGCTCTGAGGACACCGCTGTGTACTATTGT | ATTATTGTCAACAGTCATACAGCTCTCCAAGG |
| | GCCCGCGGTCCCTTTCCTAGAGGACGGCTCGA | ACGTTCGGCCAGGGTACAAAGGTTGAGATCAA |
| | CCTGTGGGGACAAGGCACACTTGTCACAGTGT | G (SEQ ID NO: 685) |
| | CCTCC (SEQ ID NO: 684) | |
| TMPRSS4 | CAGGTCCAGCTGGTCCAGTCAGGCGCCGAAGT | GACATACAGATGACACAGTCTCCTAGTTCCTT |
| Ab53 | GAAAAAGCCTGGGGCGTCCGTGAAAGTGTCCT | GTCAGCTTCGGTTGGCGATAGAGTAACCATTA |
| | GCAAAGCCTCCGGCTATACATTTACCCGATAC | CATGCAGGGCCTCTAGATCAATCAACAGGTGG |
| | TACATGCATTGGGTGCGGCAGGCTCCAGGCCA | TTGGCGTGGTATCAACAGAAACCCGGGAAAGC |
| | AGGGCTGGAATGGATGGGGATAATCAATCCCA | ACCAAAACTGCTGATCTACGGAGCATCAACTT |
| | CAGGCGGGTCTACATCGTATGCACAGAAGTTC | TACAGAGTGGCGTGCCTAGCCGTTTTTCTGGC |
| | CAAGGGCGCGTCACTATGACTCGAGACACCTC | AGCGGTAGTGGTACTGACTTCACACTTACGAT |
| | TACTAGCACGGTCTACATGGAACTAAGTAGCC | TAGTAGCCTCCAGCCGGAGGATTTCGCAACAT |
| | TCCGCAGCGAGGATACCGCCGTGTATTACTGC | ACTATTGTCAACAGAGCTACAGCACTCCCACC |
| | GCTAGAGGCAGGACCTGGATTCAATCTAGCCT | |
| | GGGGTATTGGGGTCAGGGAACACTTGTTACCG | TTTGGCGGCGGCACGAAAGTTGAGATCAAG |
| | TGTCCTCC (SEQ ID NO: 686) | (SEQ ID NO: 687) |
| TMPRSS4 | CAAGTACAGTTAGTGCAGAGCGGAGCCGAAGT | GACATTCAGATGACCCAGTCACCTTCTTCACT |
| Ab54 | TAAAAAACCTGGGGCGTCAGTCAAAGTCTCAT | GTCAGCCTCTGTGGGCGACCGGGTTACAATTA |
| | GCAAGGCCTCCGGCTATACCTTCACCTCATAC | CATGCAGAGCTTCGCAGGGAATCTCCAACTAT |
| | TACATGCAGTGGGTTCGGCAAGCTCCCGGGCA | CTGGCTTGGTATCAGCAAAAGCCCGGCAAAGC |
| | GGGCCTGGAGTGGATGGGCTGGATGAACCCTA | ACCCAAGCTCCTTATCTACGCAGCGAGCAGTT |
| | ATTCCGGCAATACTGGTTATGCACAGAAGTTC | TGCAGTCTGGGGTACCCAGTAGGTTTAGCGGG |
| | CAGGGCCGCGTGACTATGACCAGAGATACCTC | TCTGGGAGTGGCACAGATTTTACTCTGACGAT |
| | CACTTCCACCGTCTACATGGAGCTAAGCTCCC | AAGTAGCCTTCAGCCAGAGGATTTCGCCACGT |
| | TCCGTAGCGAAGACACTGCTGTGTACTATTGT | ACTATTGTCAACAGTCCTACTCGATTCCATTC |
| | GCACGAGTGCGCATCGGGTGGCTGCAGAGTCC | ACGTTTGGGCCAGGGACAAAAGTCGACATCAA |
| | TCCGTTGTACTGGGGTCAAGGGACACTCGTGA | G (SEQ ID NO: 689) |
| | CAGTGTCCAGC (SEQ ID NO: 688) | |
| TMPRSS4 | CAAGTGCAGCTCGTTCAGAGCGGCGCGGAGGT | GACATTCAGATGACTCAGAGTCCATCGTCACT |
| Ab55 | CAAGAAGCCTGGCGCCTCAGTCAAAGTCTCTT | GTCAGCCAGCGTTGGGGATAGAGTGACCATCA |
| | GCAAGGCCTCCGGCTATACTTTCACCACGTAT | CTTGCAGGGCTAGTCAAAGTATCTCCTCCTGG |
| | TACATGCATTGGGTGCGGCAGGCTCCCGGCCA | CTCGCATGGTACCAGCAGAAACCTGGGAAGGC |
| | GGGCCTGGAATGGATGGGAATTATTAATCCGA | TCCTAAACTGCTGATATACGCAGCGTCCTCCC |
| | GCGGCGGGAGTACAAGCTACGCTCAGAAATTC | TTCAGTCTGGAGTGCCCTCGAGATTTAGCGGC |
| | CAGGGACGGGTGACTATGACCCGAGACACCAG | TCTGGCTCCGGCACAGATTTCACCCTAACAAT |
| | CACATCTACTGTCTACATGGAGCTGAGTAGCT | ATCCAGCTTGCAGCCCGAAGATTTTGCCACCT |
| | TGCGCTCAGAGGACACCGCCGTGTACTATTGT | ATTACTGTCAACAGTCTTACTCTACACCAAGG |
| | GCACGCGGGAGGAGCTGGTATAGAAGCAACGT | ACGTTTGGTCAAGGGACCCGTTTAGAAATCAA |
| | AGACTATTGGGGACAAGGGACACTTGTAACAG | G (SEQ ID NO: 691) |
| | TGTCAAGT (SEQ ID NO: 690) | |
| TMPRSS4 | CAGGTTCAACTGGTCCAGTCTGGGGCAGAAGT | GACATTGTCATGACTCAAAGTCCATTGAGTCT |
| Ab56 | GAAGAAGCCTGGAGCTTCTGTTAAAGTGTCCT | GCCAGTGACACCTGGGGAGCCCGCGAGCATCT |
| | GCAAGGCCTCCGGTCACACCTTCACTCGATAT | CTTGTAGGAGCAGCCAGTCCCTCCTGCACTCC |
| | TACATGCATTGGGTTCGGCAGGCACCTGGCCA | AACGGCTATAACTATCTCGACTGGTACCTACA |
| | GGGTTTGGAGTGGATGGGGTGGATTAATCCGA | GAAACCCGGGCAGAGCCCTCAGTTACTGATCT |
| | ACTCCGGGAATACTGGGGACGCTCAGAAATTT | ACCTTGGGTCGAATAGGGCCTCTGGGGTGCCA |
| | CAGGGCCGCGTGACCATGACACGGGATACCAG | GATAGATTCTCAGGATCTGGAAGTGGCACTGA |
| | CACCAGCACCGTCTACATGGAACTCAGCTCCC | TTTCACACTGAAGATAAGTAGAGTCGAGGCCG |
| | TGCGCTCCGAAGATACGGCAGTGTACTATTGC | AGGATGTTGGCGTCTACTATTGTATGCAGGCC |
| | GCTAGGGACAGAGGCATAGTGGTGGTGCCCGC | CTTCAGACACCCATTACGTTTGGCCAAGGCAC |
| | TGCCATCGGAGGCATGGACGTATGGGGACAAG | TCGTCTGGAGATCAAG (SEQ ID NO: 693) |
| | GCACCATGGTCACAGTGTCAAGT (SEQ ID | |
| | NO: 692) | |
| TMPRSS4 | CAAGTGCAGCTCGTCCAATCTGGGGCTGAAGT | GACATTGTTATGACTCAAAGTCCCTTGTCCCT |
| Ab57 | GAAAAAGCCTGGAGCCTCCGTGAAGGTGTCCT | GCCCGTGACTCCTGGCGAACCAGCCTCAATCT |
| | GCAAGGCCTCCGGCTACACTTTCACGGGCTAT | CCTGTCGAAGCTCTCAGAGCCTTCTCCACTCT |
| | TTCATGCATTGGGTGCGGCAGGCACCCGGGCA | AATGGCTATAACTATCTGGACTGGTACCTTCA |
| | GGGACTGGAGTGGATGGGCAGGATTAATCCCA | AAAACCGGGCCAGAGTCCTCAGCTCCTAATCT |
| | ACTCCGGCGGGACAAATTACGCGCAGAAGTTT | ACTTGGGATCTAACAGGGCCTCTGGGGTGCCA |
| | CAGGGCCGCGTTACCATGACTAGAGATACCAG | GATAGGTTTAGCGGTAGTGGCAGCGGCACAGA |
| | CACTTCAACCGTTTACATGGAGCTGAGCAGTC | TTTCACCCTGAAAATTTCGCGGGTAGAAGCAG |
| | TGCGCAGCGAGGACACGGCTGTCTACTATTGC | AGGATGTGGGTGTCTACTATTGTATGCAGGGA |
| | GCTAGAGGAAAAGGGCGATATTTCGACCTGTG | ACACATTGGCCAATAACCTTTGGGCAGGGAAC |
| | GGGTAGAGGCACACTCGTAACAGTCTCCAGT | CCGTTTAGAGATCAAG (SEQ ID NO: 695) |
| | (SEQ ID NO: 694) | |
| TMPRSS4 | CAGGTCCAACTGGTCCAGTCTGGGGCAGAAGT | GACATTGTGATGACCCAGTCTCCGCTGTCACT |
| Ab58 | GAAAAAACCCGGCGCCTCCGTAAAAGTGTCCT | CCCAGTGACACCTGGCGAACCCGCTTCAATAA |
| | GCAAGGCCTCCGGCTACACTTTCACCCGATAC | GTTGTAGAAGCAGTCAGTCTTTGCTGCATAGC |
| | TATCTCCACTGGGTCCGGCAGGCTCCCGGTCA | AACGGCTACAACTATCTAGATTGGTACTTGCA |
| | GGGCTTAGAGTGGATGGGGTGGGTTAGTGCAT | GAAGCCTGGGCAGTCGCCACAACTGCTGATCT |
| | ACAATGGAAATACAAACTATGCGCAGAAATTC | ACCTGGGCAGCAATAGGGCATCTGGGGTGCCT |
| | CAAGGGCGCGTGACCATGACCCGAGACACCAG | GACCGCTTTAGCGGCAGCGGTAGTGGCACAGA |
| | CACTTCTACTGTCTATATGGAGCTTTCCTCCC | CTTCACGCTGAAAATTAGCCGTGTAGAGGCCG |
| | TCAGGAGTGAGGATACTGCCGTGTACTATTGC | AAGATGTTGGGGTCTACTATTGTATGCAGGCC |
| | GCTAGAGGTTACTGCTCAGGCGGGTCATGTTA | CTCCAGACTCCATTAACATTTGGACAAGGCAC |
| | TTGGTTTGATCCCTGGGGTCAAGGGACGCTTG | AAAGGTTGAGATCAAG (SEQ ID NO: 697) |
| | TGACCGTGTCCTCC (SEQ ID NO: 696) | |
| TMPRSS4 | CAGGTTCAGCTAGTTCAGAGCGGAGCCGAGGT | GAAATCGTCATGACCCAGTCTCCCGCCACACT |
| Ab59 | CAAGAAGCCCGGAGCGTCTGTGAAAGTGTCCT | GAGTGTATCGCCTGGGGAGCGCGCCACTCTGA |
| | GCAAAGCCTCCGGAGGCACATTCTCCTCCTAT | GTTGTAGAGCAAGCCAGAGCGTGTCGTCCAAT |
| | ACTTTGAGTTGGGTTCGGCAGGCACCTGGCCA | TACCTGGCTTGGTATCAGCAAAAGCCTGGGCA |
| | GGGCCTGGAGTGGATGGGATGGATTCACCCGA | GGCACCCAGGTTGCTTATCTACGGCGCGTCAA |
| | AAAGCGGCGTGACCAAGAATGCACAGAAATTC | CTAGGGCTACCGGGATACCAGCCCGTTTTTCC |
| | CAAGGGCGGGTGACTATGACCCGAGATACTTC | GGGTCTGGTTCAGGGACCGAATTCACGCTCAC |
| | CACGTCTACCGTCTACATGGAACTCAGCTCAC | AATTAGCAGTTTACAGAGCGAAGATTTTGCCG |
| | TGCGCTCAGAGGACACCGCTGTGTACTATTGC | TGTACTATTGTCAACAATACGGGACACTTCCA |
| | GCTAGAGGCTGGGTGTACGGCAGGATGGACGC | TATACCTTTGGCCAAGGCACAAAAGTGGAGAT |
| | CTGGGGTCAGGGTACTACGGTCACAGTCAGTA | CAAG (SEQ ID NO: 699) |
| | GC (SEQ ID NO: 698) | |
| TMPRSS4 | CAGGTCCAGCTCGTGCAGTCCGGAGCAGAAGT | GAAATAGTGATGACCCAGTCACCCGCCACCCT |
| Ab60 | TAAGAAGCCTGGCGCGTCTGTGAAAGTTTCCT | GAGTGTTAGCCCTGGGGAAAGGGCAACCCTGA |
| | GCAAGGCCTCAGGTTACTCCTTCACCACCTAT | GTTGTAGAGCGTCGCAGTCGGTGTCCTCCAAC |
| | TACATCCACTGGGTGCGGCAGGCTCCCGGGCA | ACCCTGGCATGGTATCAGCAAAAACCCGGGCA |
| | GGGCTTGGAGTGGATGGGTATTATTAACCCAT | GGCTCCTAGGCTCCTAATCTACGGCGCCTCTA |
| | CTGGCGGGTCTACAAGCTACGCTCAAAAGTTT | CTCGTGCCACTGGCATTCCCGCCAGATTTAGC |
| | CAGGGTCGGGTCACTATGACACGAGATACTAG | GGGAGCGGGTCCGGCACAGAGTTTACGCTTAC |
| | CACATCTACCGTCTACATGGAGCTGAGCAGTC | AATAAGTTCCTTGCAGTCAGAAGATTTCGCAG |
| | TGCGCAGCGAGGACACCGCTGTGTACTATTGC | TGTACTATTGTCAACAATATGGATCTTCTCCG |
| | GCCCGCGGCGGCTACTATGGCTCCGGATACAA | TTAACGTTCGGGCCAGGCACAAAAGTGGACAT |
| | TTCAGTCGGTTATTGGGGACCAGGGACGCTTG | CAAG (SEQ ID NO: 701) |
| | TAACAGTATCAAGC (SEQ ID NO: 700) | |
| TMPRSS4 | CAGGTTCAGTTGGTCCAGTCTGGCGCGGAAGT | GAGATTGTTATGACTCAGTCTCCGGCCACACT |
| Ab61 | AAAAAAGCCTGGAGCGTCAGTGAAAGTGTCGT | GAGTGTTTCTCCTGGCGAAAGGGCAACTCTCA |
| | GCAAGGCTAGTGGCTATACCTTCACCAATTAC | GTTGCAGAGCATCTCAAAGCGTGTCCTCCTAC |
| | TACATGCATTGGGTGCGGCAGGCACCAGGCCA | CTAGCTTGGTATCAACAGAAACCCGGGCAGGC |
| | GGGCTTGGAGTGGATGGGCTGGATGAATCCCA | ACCTAGGCTGCTCATCTACGGCGCCTCGACCA |
| | ACTCCGGGAACACCGGCTATGCTCAGAACCTG | GAGCCACCGGGATTCCCGCCCGTTTTAGCGGG |
| | CAAGGGCGAGTCACAATGACCCGAGATACCAG | TCTGGGAGCGGGACTGAGTTCACCTTAACAAT |
| | CACTAGCACTGTCTACATGGAGCTGTCCAGCC | AAGTAGCCTTCAGTCAGAAGACTTCGCTGTGT |
| | TGCGCTCAGAGGACACCGCCGTGTACTATTGT | ACTATTGTCAACAGTATGATATATCTGTGACG |
| | GCCCGCGGGAGGACTTGGTTTAGATCCGGAAT | TTTGGGCCAGGCACAAAGGTCGATATCAAG |
| | GGACGTGTGGGGTCAGGGAACGACGGTCACAG | (SEQ ID NO: 703) |
| | TGTCCAGT (SEQ ID NO: 702) | |
| TMPRSS4 | CAGGTCCAGCTGGTTCAGTCTGGCGCGGAAGT | GACATTGTGATGACCCAGTCACCAGATAGCCT |
| Ab62 | GAAAAAGCCGGGCGCCTCCGTCAAAGTGTCCT | TGCAGTGTCCCTGGGAGAAAGGGCTACCATTA |
| | GCAAGGCCTCCGGTTACACCTTCACTGACTAT | ATTGCAAGAGCAGTCAGTCAGTGCTCTACTCA |
| | TACATCCACTGGGTCCGGCAAGCACCCGGGCA | AGCAACAACAAAAACTATTTGGCTTGGTACCA |
| | GGGCCTGGAATGGATGGGGTGGATTTCTACTT | ACAGAAGCCCGGGCAGCCTCCTAAGCTGCTGA |
| | ATAATGGGAACACGAATTATGCACAGAAACTC | TCTACTGGGCCTCTACAAGGGAGTCTGGGGTG |
| | CAGGGCCGCGTCACTATGACCCGGGATACAAG | CCAGATCGCTTTTCCGGGTCCGGCTCCGGGAC |
| | CACTAGCACCGTCTACATGGAGCTAAGTAGCT | AGACTTCACGCTTACAATAAGTAGTCTCCAGG |
| | TGCGTAGCGAGGACACCGCTGTGTATTACTGT | CTGAGGACGTAGCCGTGTACTATTGTCAACAA |
| | GCCAGAGGCATGGTGCGAGGCATGGATGTATG | TACTATACTACACCCTGGACCTTTGGCCAGGG |
| | GGGACAAGGAACAATGGTTACTGTTTCTAGT | AACCAGATTAGAGATCAAG (SEQ ID NO: 705) |
| | (SEQ ID NO: 704) | |
| TMPRSS4 | CAAGTGCAGCTGGTGCAGTCTGGAGCGGAAGT | GACATTGTTATGACACAGTCTCCCGATAGCCT |
| Ab63 | GAAAAAACCTGGCGCCAGTGTGAAAGTGTCCT | CGCCGTTAGTCTCGGCGAGCGTGCCACGATAA |
| | GCAAGGCCTCCGGTTATACTTTCACGGGCTAT | ACTGCAAATCTTCTCAGTCCGTGTTGTACTCA |
| | CGGATGCATTGGGTTCGGCAGGCACCTGGGCA | AGCAACAACAAGAATTATCTGGCATGGTACCA |
| | AGGACTAGAGTGGATGGGCGTAATTAATCCAA | GCAGAAACCTGGGCAACCACCCAAACTGCTGA |
| | ATACTGGGACCGCTCGCTTTGCTCAGAAGTTT | TCTACTGGGCCTCAACCCGCGAATCTGGGGTG |
| | CAGGGACGAGTCACAATGACTAGGGATACATC | CCGGATAGATTCAGCGGGAGCGGCTCCGGGAC |
| | AACCAGCACTGTCTACATGGAGCTGAGCAGCC | AGATTTCACCCTTACTATCTCGAGTTTGCAGG |
| | TCAGGAGCGAAGACACCGCGGTCTACTATTGT | CTGAGGACGTTGCCGTCTACTATTGTCAACAG |
| | GCTTCTGTGGGCGTCTACTGGTATTTTGACCT | TACTATTCAGCACCCTTAACCTTCGGCGGCGG |
| | GTGGGGTAGAGGCACACTTGTAACCGTGTCCT | CACAAAAGTGGAAATCAAG (SEQ ID NO: 707) |
| | CC (SEQ ID NO: 706) | |
| TMPRSS4 | CAGGTTCAGCTGGTACAGTCAGGCGCCGAAGT | GACATTGTAATGACTCAGAGCCCAGATAGTCT |
| Ab64 | GAAAAAACCGGGTGCCAGCGTCAAGGTGTCCT | TGCAGTGTCCTTGGGCGAGAGGGCTACAATCA |
| | GCAAGGCCTCCGGCTACACCTTCACCGGATAT | ACTGCAAGAGCAGTCAGAGCGTGCTCTACTCG |
| | TACATGCATTGGGTCCGGCAGGCACCCGGCCA | TCTAACAACAAAAATTACCTTGCGTGGTATCA |
| | AGGGCTGGAATGGATGGGGATGATTAATCCTA | ACAGAAACCCGGGCAGCCTCCCAAACTGCTGA |
| | GTGGTGGCGGGACCACATACGCTCAGAAGTTC | TCTACTGGGCTTCTACTAGAGAGTCTGGCGTG |
| | CAAGGGCGGGTTACGATGACTCGAGACACCAG | CCTGATCGTTTTAGCGGTTCCGGGTCTGGGAC |
| | CACGTCTACCGTCTACATGGAACTGTCCAGCC | AGACTTCACGTTAACAATAAGTAGCCTCCAGG |
| | TACGCTCTGAGGATACTGCCGTGTACTATTGT | CTGAGGACGTCGCCGTGTACTATTGTCAACAG |
| | GCAAGGGACAGGAGATCAATGATTACCTTTCG | TACTATTCAACACCATATACTTTTGGCCAAGG |
| | CACAGATTATTGGGGTCAGGGAACTCTCGTTA | CACCAAGTTGGAGATCAAG (SEQ ID NO: 709) |
| | CCGTGTCCTCC (SEQ ID NO: 708) | |
| TMPRSS4 | CAGGTCCAACTGGTCCAGTCTGGCGCGGAGGT | GACATTGTGATGACTCAGAGTCCGGATAGCCT |
| Ab65 | TAAAAAGCCTGGGGCTTCAGTCAAAGTGTCGT | TGCAGTGTCTTTGGGCGAGCGCGCCACTATTA |
| | GTAAGGCCTCGGGCTATACATTCACCGGCTAT | ATTGCAAATCCTCCCAGTCCGTTCTCTACAGC |
| | TACATGCATTGGGTCCGTCAGGCACCTGGGCA | TCAAATAACAAGAACTATCTCGCATGGTACCA |
| | AGGGCTGGAATGGATGGGTTGGATGAATCCGA | GCAGAAGCCAGGACAGCCACCAAAACTGCTGA |
| | ACTCCGGGAATACCGGTTATGCCCAAAAATTC | TATACTGGGCTAGTACCAGACAGTCTGGCGTG |
| | CAAGGGAGAGTAACTATGACACGAGACACCAG | CCCGACAGGTTTTCAGGGTCCGGGAGCGGCAC |
| | CACTAGCACCGTGTATATGGAACTCAGCTCTC | AGACTTCACGCTAACGATAAGTAGTTTACAGG |
| | TGAGGAGCGAGGACACAGCCGTGTATTACTGC | CCGAGGATGTCGCTGTGTATTACTGCCAGCAG |
| | GCGGGCCGGAAATGGCTGGGCTTGGATTTCTA | TACTACTCTACACCCTGGACCTTTGGGCAAGG |
| | CAACTGGTTTGATCCTTGGGGTCAGGGAACTC | CACAAAGGTTGAAATCAAG (SEQ ID NO: 711) |
| | TTGTAACGGTGTCCAGT (SEQ ID NO: 710) | |
| TMPRSS4 | CAGGTTCAACTGGTACAGAGCGGCGCGGAAGT | GAGATTGTGATGACTCAGAGCCCTGCTACACT |
| Ab66 | GAAAAAACCCGGCTCGTCAGTTAAAGTGTCCT | GAGTGTATCTCCTGGGGAACGGGCCACCCTCA |
| | GTAAGGCCTCCGGCGGCACATTCTCCTCCTAC | GTTGTAGAGCCTCTCAGAGCGTCAACTCTAGA |
| | GCTATCTCCTGGGTCCGGCAAGCACCCGGGCA | TTTCTAGCCTGGTATCAGCAAAAGCCGGGCCA |
| | AGGTCTGGAGTGGGTGGGTGGCATAATGCCCA | GGCACCAAGGTTGCTCATCTACGGCGCATCAA |
| | TATTCGGGACAGCGAATTACGCTCAGAAGTTT | CTAGGGCAACCGGGATTCCTGCCCGCTTCTCA |
| | CAGGGAAGAGTGACTATTACCGCCGATGAATC | GGCAGCGGGTCAGGGACTGAGTTTACACTTAC |
| | TCCATCTACGGCTTATATGGAACTCTCCAGTC | GATCAGTAGCTTACAGAGCGAGGACTTTGCCG |
| | TGCGCAGCGAGGACACCGCTGTGTACTATTGC | TCTACTATTGCATGCAGGGAACTCACTGGCCA |
| | GCCACCGGGCGTCGAGAGTTGCTGAACTGGGG | TACACCTTCGGCCAAGGGACAAAAGTGGAAAT |
| | TCAGGGAACACTTGTTACCGTGTCCAGC (SEQ | CAAG (SEQ ID NO: 713) |
| | ID NO: 712) | |
| TMPRSS4 | CAGGTCCAGCTAGTTCAGTCTGGGGCAGAAGT | GACATTGTAATGACCCAGTCTCCCGATAGCCT |
| Ab67 | CAAGAAACCGGGTAGCTCAGTGAAGGTGTCCT | TGCCGTGTCCTTGGGCGAAAGGGCAACCATAA |
| | GCAAAGCAAGCGGCTACACTTTCACCTCATAT | ATTGCAAGTCCTCCCAGTCCGTCTTGTACTCG |
| | GATATTAATTGGGTGCGACAGGCACCAGGCCA | AGTAACAACAAAAACTATCTCGCGTGGTATCA |
| | AGGGCTGGAATGGATGGGCGGCATCATTCCCA | GCAAAAGCCCGGGCAACCTCCTAAACTGCTGA |
| | TCTTCGGGACAGCCAATTACGCTCAGAAGTTT | TCTACTGGGCTAGTACCAGAGAGAGCGGCGTG |
| | CAGGGACGGGTTACGATTACTGCCGATGAAAG | CCAGACCGTTTTTCTGGGAGTGGTAGCGGGAC |
| | CACCTCTACTGCCTATATGGAGTTAAGCTCTC | TGATTTCACCCTTACGATAAGTAGTCTCCAGG |
| | TGCGCAGCGAGGACACCGCTGTGTACTATTGT | CCGAGGACGTAGCGGTGTACTATTGTCAACAG |
| | GCCACTACACCAGGCGATGCTTTCGACATATG | TACAGTGACACACCTCTGACCTTTGGGCAAGG |
| | GGGTCAGGGAACAATGGTTACAGTCTCATCT | CACAAAAGTGGAAATCAAG (SEQ ID NO: 715) |
| | (SEQ ID NO: 714) | |
| TMPRSS4 | GAAGTGCAACTGCTCGAATCTGGCGGTGGGCT | GACATCCAGATGACTCAGTCTCCGAGCAGCTT |
| Ab68 | GGTCAAACCTGGTGGATCTCTTCGGCTGTCAT | GAGTGCTTCTGTTGGGGATCGCGTTACCATTA |
| | GCGCAGCCTCCGGGTTTACCTTCTCCTCCTCC | CATGCAGGGCCTCCCAGTCAATCTCTCGTTAC |
| | TGGATGCATTGGGTGCGACAGGCACCCGGGAA | TTAAATTGGTATCAGCAAAAACCCGGCAAGGC |
| | AGGCCTGGAGTGGGTGTCAGCCATCGGGACTG | ACCCAAGCTGCTGATTTACGCGGCGAGCAGTC |
| | CCGGTGATACCTATTACCCTGGCTCGGTGAAA | TCCAAAGCGGAGTGCCATCCAGATTCAGCGGG |
| | GGGAGGTTCACCATTAGCCGGGATGACTCCAA | AGCGGGTCGGGCACCGATTTTACCCTTACAAT |
| | GAATACGCTGTACTTGCAGATGAACTCACTCA | AAGTAGTTTGCAGCCAGAGGACTTCGCTACAT |
| | AGACTGAGGACACGGCTGTCTATTACTGTGCC | ACTATTGTCAACAGTCCTATAGCAACCCACCT |
| | AGAGTGCGACTCGGCCACTTTGACCTGTGGGG | ACTTTTGGCCAGGGAACTAAGCTAGAAATCAA |
| | ACGCGGGACACTTGTAACAGTCAGCTCA (SEQ | G (SEQ ID NO: 717) |
| | ID NO: 716) | |
| TMPRSS4 | GAAGTTCAACTGCTCGAGTCCGGCGGTGGGCT | GACATTCAGATGACCCAGTCTCCAAGTAGCCT |
| Ab69 | TGTTCAGCACGGCGGCTCTTTGCGGCTGTCCT | AAGTGCAAGCGTCGGCGACAGAGTAACAATTA |
| | GTGCTGCGAGCGGATTTGCCTTCTCCTCCTAC | CATGCAGAGCTTCGCAAGGGATCTCAAGTTGG |
| | GTCCTGCATTGGGTTCGGCAGGCACCTGGGAA | CTCGCCTGGTACCAGCAGAAACCTGGGAAAGC |
| | GGGCCTGGAGTGGGTGTCCAGTATCAGTTCCA | ACCTAAGCTTCTCATATACCAGGCCTCAAACA |
| | GTAGCAGCTACATCTACTACGCCGACTCCGTG | AGGATACTGGGGTGCCATCTAGGTTTTCTGGC |
| | AAAGGGCGATTCACCATCTCACGCGACAATTC | TCTGGCTCGGGCACGGACTTCACCCTCACAAT |
| | AAAAAACACACTGTACTTGCAGATGAACAGCC | AAGCAGCTTACAGCCCGAAGATTTTGCCACGT |
| | TTAGGGCCGAGGATACTGCTGTGTACTATTGC | ATTATTGTCAACAGTCATATAGGATTCCGTGG |
| | GCTCGTGGGGATCGCTATCCCGGCCTGCCCAA | ACTTTCGGGCAGGGAACCAAGGTGGAAATCAA |
| | TTATTGGGGTCAGGGAACATTAGTAACCGTGT | G (SEQ ID NO: 719) |
| | CTAGT (SEQ ID NO: 718) | |
| TMPRSS4 | GAGGTCCAGCTTCTGGAATCAGGCGGCGGGCT | GACATTCAGATGACTCAGAGTCCAAGCAGCCT |
| Ab70 | GGTCCAGCCTGGTGGTTCCTTACGGTTGTCCT | GAGCGCCAGCGTTGGCGATAGAGTGACCATTA |
| | GCGCTGCCTCCGGGTTTGCGTTTAGTGGCACT | CCTGCAGAGCGTCACAATCCATCTCGGGTTGG |
| | TGGATGCAGTGGGTGCGGCAGGCACCGGGCAA | CTGGCCTGGTACCAGCAGAAGCCTGGGAAAGC |
| | GGGCCTGGAGTGGGTTTCTGACATTTCCGGGT | ACCCAAGCTCCTCATCTACGCAGCTTCGACCC |
| | CCAGTAGGGACACCAACTACGCTGACAGTGTA | TACGTGATGGGGTCCCATCTCGCTTTAGCGGG |
| | AAGGGACGATTCACAATAAGCAGGGACAACTC | AGTGGCTCTGGCACTGATTTCACGCTCACAAT |
| | TAAAAATACGCTGTACCTCCAGATGAACTCTC | AAGCAGCCTTCAGCCCGAGGACTTCGCCACCT |
| | TGCGCGCTGAAGATACAGCCGTGTACTATTGT | ACTATTGTCAACAGGCCAATTCCTTTCCCTTA |
| | GCAAAAGATCACTGGGATTCATATGGGTATCT | ACCTTCGGACAAGGGACAAAAGTGGAAATCAA |
| | GGACTATTGGGGACAAGGGACTTTGGTGACAG | G (SEQ ID NO: 721) |
| | TGTCTAGT (SEQ ID NO: 720) | |
| TMPRSS4 | GAAGTCCAACTGCTCGAGAGTGGCGGCGGGCT | GACATTCAGATGACCCAATCGCCAAGTTCCCT |
| Ab71 | TGTTCAGCCTGGCGGTTCTTTGCGGCTGTCAT | GAGTGCTTCAGTCGGCGACCGCGTAACTATTA |
| | GCGCCGCGAGCGGCTTCATGTTTGACTATTAC | CCTGTAGAGCCAGTCAAGGGATTTCTAATAAC |
| | GCCATGCATTGGGTTCGACAGGCTCCCGGGAA | CTGAATTGGTATCAACAGAAGCCCGGGAAAGC |
| | GGGCCTAGAGTGGGTGTCCTTGATCTCCTATG | TCCTAAACTGCTGATATACGCGGCATCCTCCC |
| | ATGGGAGGAACAAGTACTACGCCGACTCAGTG | TCCAGTCAGGCGTGCCTAGTCGTTTTAGCGGG |
| | AAGGGCCGCTTCACAATCAGCCGGGATAATTC | TCTGGCTCAGGCACTGACTTTACACTCACGAT |
| | CAAAAACACCCTGTACCTCCAGATGAACAGCC | CAGCTCCTTACAGCCGGAGGATTTCGCCACTT |
| | TTAGAGCCGAAGATACCGCTGTCTACTATTGT | ATTACTGCCAGCAGGCAAATAACTTCCCAATA |
| | GCAAGGCCCGGGAGCTATTCTAGATTTCAGCA | ACGTTCGGGCAGGGAACCAAAGTGGAAATCAA |
| | CTGGGGTCAGGGAACATTAGTGACAGTGTCGT | G (SEQ ID NO: 723) |
| | CT (SEQ ID NO: 722) | |
| TMPRSS4 | GAAGTGCAGCTGCTCGAGTCGGGCGGCGGGCT | GACATACAGATGACTCAGTCTCCAAGCTCCTT |
| Ab72 | GGTGCAACCAGGAGGGTCATTACGGTTGTCAT | GTCAGCTTCTGTTGGGGACCGAGTTACCATCA |
| | GCGCTGCCTCTGGGTTTACGTTCGGAGCCTAC | CTTGTAGGGCCTCACAAAATATCTCCCGCTGG |
| | GTGATGCATTGGGTCCGGCAGGCACCTGGGAA | CTGGCTTGGTATCAGCAAAAGCCGGGCAAAGC |
| | AGGCCTGGAGTGGGTGTCCTCCATTAGTGGCG | TCCCAAGCTGCTGATCTACGCGGCGAGTAGCC |
| | GCAGCACTTACTACGCCGACTCCGTTAAAGGG | TCCAGTCGGGAGTACCCAGCAGATTTAGCGGC |
| | AGATTCACAATAAGCAGGGACAACTCAAAGAA | TCCGGGTCTGGGACCGATTTCACCCTAACAAT |
| | CACGCTGTACCTCCAGATGAATAGCCTTCGCG | CAGTAGCCTCCAGCCCGAGGATTTTGCCACTT |
| | CAGAAGATACCGCCGTGTACTATTGCGCCAGG | ATTATTGTCAACAGGCAATTAGCTTCCCTTTA |
| | CACCCAGTCCGTGGCGTGATTGGGGCAGGCTG | ACCTTTGGCGGTGGCACCAAGGTCGAAATCAA |
| | GTTCGATCCTTGGGGTCAGGGTACACTTGTAA | G (SEQ ID NO: 725) |
| | CAGTGTCCAGT (SEQ ID NO: 724) | |
| TMPRSS4 | GAGGTTCAGCTTCTCGAGTCGGGCGGAGGGCT | GACATTCAGATGACACAGAGTCCATCTTCACT |
| Ab73 | AGTTCAGCCTGGCGGGTCTTTGCGTCTGAGCT | GTCAGCTTCGGTCGGCGACAGAGTGACTATTA |
| | GTGCCGCCAGCGGCTTTACCTTCTCCTCATAC | CATGTCGAGCATCCCAAGGAATCTCCAATTCT |
| | GCCATGCATTGGGTTCGACAGGCTCCTGGGAA | CTGGCGTGGTATCAACAGAAACCCGGCAAAGC |
| | GGGCCTGGAGTGGCTCGCCGTAATCTCCTTCG | TCCCAAGCTGCTGATATACAGCGCAGTGAACC |
| | ACGGGTCTATCAGACACTATGCGGACTCCGTG | TCCAGAGCGGAGTGCCCTCACGCTTTTCCGGA |
| | AAAGGCAGGTTCACCATTTCTCGGGACAATTC | AGTGGCTCTGGGACTGATTTTACACTTACGAT |
| | CAAGAACACGCTGTACTTGCAGATGAACAGTC | AAGTAGCCTCCAGCCTGAAGATTTCGCTACAT |
| | TGCGGGCAGAAGATACCGCCGTGTACTATTGC | ATTACTGCCAACAGGCAAATAGCTTTCCATTA |
| | GCCAAACCAAAGGCCTCCAGCGGGCCGCGCTT | ACTTTCGGCGGCGGAACAAAAGTCGAAATCAA |
| | GATAGATTACTGGGGACAAGGGACTCTTGTGA | G (SEQ ID NO: 727) |
| | CCGTCAGCTCA (SEQ ID NO: 726) | |
| TMPRSS4 | GAAGTGCAGCTGCTCGAAAGTGGAGGCGGGCT | GACATTCAGATGACTCAGTCGCCATCATCGTT |
| Ab74 | GGTTCAACCCGGAGGGAGCTTGCGGCTCTCAT | GTCCGCTAGTGTCGGCGATAGAGTGACTATTA |
| | GCGCTGCGAGCGGTTTTACCTTCTCCTCCTAT | CCTGCCGTGCCTCTCAGAGCGTGTCCTCCTGG |
| | GCCATGCATTGGGTTCGGCAGGCGCCCGGGAA | CTGGCATGGTATCAACAGAAACCTGGGAAGGC |
| | AGGCCTGGAGTGGGTGTCCTCCATCTCAAGTA | ACCGAAGCTGCTCATCTACGATGCCTCTAGTC |
| | GCTCTACCTACATTCACTACGCCGATAGCGTC | TACAGTCTGGCGTACCCTCCCGCTTTAGCGGC |
| | AAGGGTAGATTCACGATCTCCCGCGACAACAG | TCTGGGTCTGGCACCGATTTCACTCTTACAAT |
| | CAAAAATACCCTGTACCTCCAGATGAACTCCT | AAGCAGTTTACAGCCTGAGGACTTCGCCACAT |
| | TACGAGCCGAGGACACTGCTGTGTACTATTGT | ATTACTGTCAACAGGCTAAAAGTTTTCCACCT |
| | GCAAGGGTGGGGAGGTATTACGGCAGCGGCTC | ACGTTTGGCCAAGGGACAAAGGTCGAAATCAA |
| | ATCACTGGTAGACTATTGGGGACAAGGGACAC | G (SEQ ID NO: 729) |
| | TTGTCACCGTGTCTAGC (SEQ ID NO: 728) | |
| TMPRSS4 | GAAGTGCAGTTACTCGAGAGCGGCGGCGGATT | GACATTCAGATGACCCAGTCTCCATCTTCACT |
| Ab75 | GGTCCAGCCTGGCGGTAGCCTGCGGTTGTCAT | GTCAGCTAGTGTTGGGGATAGAGTCACTATAA |
| | GCGCTGCGAGTGGGTTCACCTTCTCCTCATAC | CTTGCAGGGCCTCTCAAGGGATTAGGAACGAC |
| | GCCATGTCGTGGGTCCGGCAGGCACCCGGCAA | CTTAACTGGTATCAGCAAAAGCCCGGGAAAGC |
| | AGGCCTGGAGTGGGTGTCCTCCATCAGTAGTG | ACCGAAGCTCCTGATTTACGCAGCGACAAGAC |
| | CTTCTAGCTACAAGTATTATGCCGACTCCGTG | TGCAAAGCGGCGTACCTAGCAGATTCAGCGGG |
| | AAAGGGCGATTTACCATCTCCCGAGACAATTC | TCGGGTTCCGGGACTGATTTTACCCTCACAAT |
| | CAAGAATACGCTGTACCTCCAGATGAACTCTC | CAGCTCCCTACAGCCAGAGGACTTCGCCACTT |
| | TTCGCGCTGAAGATACCGCCGTGTACTATTGT | ATTACTGTCAACAGGCCCATAGTTTTCCCTAT |
| | GCAAGGGATATATACTCCAGCGGATGGCGCGG | AGCTTTGGGCAGGGAACACGTCTGGAAATCAA |
| | CTACTACTACTATGGGATGGATGTTTGGGGAC | G (SEQ ID NO: 731) |
| | AAGGGACCACGGTGACAGTGTCAAGT (SEQ | |
| | ID NO: 730) | |
| TMPRSS4 | GAAGTCCAACTCCTGGAGAGTGGCGGCGGGCT | GACATTGTGATGACACAAAGCCCAGATTCTCT |
| Ab76 | GGTACAGCCCGGCGGATCTTTGCGGCTGAGTT | TGCTGTCAGCCTCGGAGAGAGAGCCACGATCA |
| | GTGCTGCAAGCGGGTTTACCTTCTCCTCATAT | ATTGCAAATCCTCCCAGTCTGTGCTGTACTCA |
| | GCCATGTCATGGGTTCGGCAGGCACCTGGGAA | TCTAACAACAAGAACTATCTCGCATGGTACCA |
| | GGGCCTAGAGTGGGTCTCAGCCATTAGCGGGT | GCAGAAACCGGGCCAGCCTCCCAAACTGCTGA |
| | CGGGTGGCAATGCGTACTATGCGGACAGCGTG | TCTACTGGGCCTCTACCCGAGCATCGGGCGTG |
| | AAGGGCCGTTTCACCATCAGCCGCGACAATGC | CCTGACCGCTTTAGCGGGTCTGGGTCTGGGAC |
| | CAAAAACAGTCTCTACTTGCAGATGAACTCCC | CGATTTCACTCTTACAATAAGTTCCTTACAAG |
| | TGAGGGCTGAGGACACTGCGGTGTACTATTGC | CAGAAGATGTCGCCGTTTATTACTGCCAGCAG |
| | GCCAAAAATAGTTGGGGCTCTTATAGGCCAAG | TATCTGAGCTTACCCTACACCTTCGGCCAGGG |
| | AGCCTTTGATATTTGGGGACAAGGGACAATGG | TACAAAAGTTGAAATCAAG (SEQ ID NO: 733) |
| | TGACTGTGTCCTCC (SEQ ID NO: 732) | |
| TMPRSS4 | CAGGTTCAGCTTGTCCAGTCTGGTGCCGAGGT | GACATTGTTATGACCCAGTCACCAGATTCGCT |
| Ab77 | GAAAAAGCCTGGCGCGTCAGTTAAAGTGTCCT | GGCAGTGTCCCTGGGCGAAAGGGCTACTATTA |
| | GCAAGGCCAGCGGGAATATCTTCACCGCACAG | ACTGTAAAAGTAGCCAGTCCGTGCTCTATAGC |
| | TACATGCATTGGGTTCGGCAGGCACCAGGCCA | AGTAACAACAAAAACTACTTGGCCTGGTACCA |
| | GGGTTTGGAATGGATGGGCTGGATGAATCCGA | ACAGAAGCCCGGGCAGCCTCCCAAACTGCTGA |
| | ATACCGTCTATACCGGGAGTGCCCAAAAGTTC | TCTACTGGGCCTCTACAAGAGAGTCTGGGGTG |
| | CAGGGCCGAGTCACTATGACAAGAGACACCAG | CCCGACCGTTTTTCTGGCTCTGGGTCCGGCAC |
| | CACATCCACTGTCTACATGGAGCTAAGCTCTC | TGACTTCACGCTTACGATAAGCAGCCTCCAGG |
| | TGCGCTCAGAAGATACCGCTGTGTACTATTGC | CTGAGGACGTAGCTGTGTACTATTGTCAACAG |
| | GCCAGGGATTGGGTCGGTGATGGGTATAATAG | TACTATACCACACCTTTCACGTTTGGCCCAGG |
| | CTTTGATTATTGGGGACAAGGCACATTAGTAA | GACAAAGGTGGACATCAAG (SEQ ID NO: 735) |
| | CTGTGTCCAGT (SEQ ID NO: 734) | |
| TMPRSS4 | GAAGTTCAGCTGCTCGAAAGCGGAGGCGGGCT | GACATCCAGATGACGCAGAGTCCTAGCTCCCT |
| Ab78 | GGTCCAGCCTGGCGGGAGTTTACGGTTGTCCT | GTCAGCCTCTGTCGGCGATAGAGTGACAATTA |
| | GTGCCGCGTCTGGTTTTACCTTCTCCTCATAC | CTTGCCGGGCGAGTCAAGATATCAAGAATTTC |
| | GGCATGAATTGGGTGCGCCAGGCACCCGGGAA | CTCGCATGGTATCAGCAAAAGCCCGGGAAAGC |
| | AGGCCTGGAGTGGGTCAGCGCTATTAGTGGTT | ACCGAAACTGCTGATCTACGCCGCCAGCAGCC |
| | CTGGTGGCAGGACTTACTACGCTGATTCAGTT | TCCAGAGCGGCGTGCCATCTCGTTTTTCAGGC |
| | AAAGGGAGATTCACCATTTCACGAGACAACGC | TCTGGGTCTGGGACAGATTTCACACTTACCAT |
| | TAAAAACAGCCTGTACTTGCAGATGAACTCCC | AAGTAGCTTACAGCCCGAGGACTTTGCCACCT |
| | TTAGGGCCGAGGACACCGCAGTGTATTACTGC | ATTACTGTCAACAGTCTTACAGCACTCCTTGG |
| | GCTAAGGGCACATATTATTCCTCGCCAAAGTA | ACGTTCGGGCAGGGAACAAAGCTAGAAATCAA |
| | TTCGTTTGACTATTGGGGACAAGGCACTCTCG | G (SEQ ID NO: 737) |
| | TAACCGTGTCCTCC (SEQ ID NO: 736) | |
| TMPRSS4 | GAAGTTCAGCTTCTCGAGAGCGGCGGCGGGCT | GACATTCAGATGACACAGAGCCCAAGCTCACT |
| Ab79 | GGTGCAACCGGGAGGTTCTTTGCGGCTGTCAT | GTCAGCTTCTGTAGGCGACCGCGTCACCATCA |
| | GTGCCGCGAGCGGGCTGACCTTCAGCTCATAT | CTTGCAGAGCCTCGCAGGGAATTTCTAATTAC |
| | CAGATGTCATCCGTCAGTCAGGCACCTGGCAA | CTAGCATGGTATCAACAGAAGCCAGGGAAAGC |
| | AGGCCTGGAGTGGGTGTCCTACATAAGCAGTG | TCCTAAGCTGCTGATCTACGCAGCAAGTAGTC |
| | CAGCCAATACTGTGTACTATGCGGACAGCGTT | TCCAGAGTGGAGTGCCGTCCAGGTTTAGCGGC |
| | AAGGGCCGATTCACGATCAGTCGGGACAACTC | AGCGGTTCAGGGACGGACTTCACCCTCACCAT |
| | CAAGAACACTCTGTACCTCCAGATGAACTCCT | AAGTTCCTTACAGCCCGAGGATTTCGCCACCT |
| | TACGCGCAGAGGATACTGCTGTGTACTATTGC | ATTATTGTCAACAGAGCTACTCTACACCCTTG |
| | GCCAGGGAAGATGAGTCTAGATCGCCTTATTG | ACATTTGGGCCAGGGACAAAAGTCGATATTAA |
| | TAGCGGCGGGTCTTGCTACCGTGCTGAATACT | G (SEQ ID NO: 739) |
| | TTCAACATTGGGGTCAGGGTACACTTGTAACC | |
| | GTGTCCTCC (SEQ ID NO: 738) | |

In some embodiments, the VH of the antibody or antigen-binding fragment that binds to TMPRSS4 is encoded by a nucleic acid comprising a sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the sequence set forth in SEQ ID NO: 583, 585, 587, 589, 591, 592, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 684, 686, 688, 690, 692, 694, 696, 698, 700, 702, 704, 706, 708, 710, 712, 714, 716, 718, 720, 722, 724, 726, 728, 730, 732, 734, 736, or 738, optionally comprising a nucleotide sequence encoding the VH CDRs encoded by SEQ ID NO: 583, 585, 587, 589, 591, 592, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 684, 686, 688, 690, 692, 694, 696, 698, 700, 702, 704, 706, 708, 710, 712, 714, 716, 718, 720, 722, 724, 726, 728, 730, 732, 734, 736, or 738, respectively. In some embodiments the VL of the antibody or antigen-binding fragment that binds to TMPRSS4 is encoded by a nucleic acid comprising a sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the sequence set forth in SEQ ID NO: 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 685, 687, 689, 691, 693, 695, 697, 699, 701, 703, 705, 707, 709, 711, 713, 715, 717, 719, 721, 723, 725, 727, 729, 731, 733, 735, 737, or 739, optionally comprising a nucleotide sequence encoding the VL CDRs encoded by SEQ ID NO: 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 685, 687, 689, 691, 693, 695, 697, 699, 701, 703, 705, 707, 709, 711, 713, 715, 717, 719, 721, 723, 725, 727, 729, 731, 733, 735, 737, or 739. Table 7 provides exemplary nucleic acid sequences encoding scFvs that bind to TMPRSS4. In some embodiments, the antibody or antigen-binding fragment that binds to TMPRSS4 comprises an scFv encoded by a nucleic acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to a nucleic acid sequence set forth in Table 7, optionally wherein the VH CDRs and the VL CDRs are identical to those in the respective sequences in Table 7.

TABLE 7
TMPRSS4 scFv Nucleotide Sequences

| Clone | scFv Nucleic Acid Sequence |
|---|---|
| TMPRSS4 | CAAGTGCAGCTCGTTCAGTCTGGGGCGGAGGTTAAAAAACCGGGCTCATCTGTGAAAGTGTCTTGC |
| Ab1 scFv | AAGGCCTCCGGCTATACCTTCACCGATTACTACATGCATTGGGTCCGGCAGGCACCCGGGCAGGGA |
| (VH-VL) | CTTGAGTGGATGGGCGGGATTATCCCAATCTTTGGGACTGCAAATTATGCTCAGAAATTCCAAGGG |
| | CGCGTAACTATTACCGCCGACGAAAGCACAAGCACCGCGTACATGGAGCTATCGAGCCTCCGTAGC |
| | GAGGACACCGCTGTGTACTATTGCGCCAAGGAAGGCGCTAATGGCTACTGGGGTCAGGGAACATTG |
| | GTAACAGTGTCCAGTGGAGGCGGCGGTTCAGGAGGTGGTGGAAGTGGAGGAGGAGGTTCCGACATT |
| | CAGATGACTCAGTCGCCAAGTTCACTGTCAGCTTCCGTCGGCGATAGAGTCACTATTACTTGTCAA |
| | GCATCCCAGGATATATCCAACTATTTGAACTGGTACCAGCAGAAGCCCGGCAAGGCACCCAAGCTG |
| | CTGATATACAAGGCCTCCTCTCTGGAATCAGGCGTGCCTAGCCGATTTTCTGGGTCTGGGAGTGGC |
| | ACAGATTTCACGCTGACAATCAGCTCCTTACAGCCTGAGGACTTCGCCACCTATTATTGTCAACAA |
| | AGCAGCAGGATCCCTCCAACCTTTGGGCAGGGAACGAAAGTTGAAATCAAG |
| | (SEQ ID NO: 740) |
| TMPRSS4 | GAAGTGCAGCTCCTAGAGTCTGGCGGCGGGCTGGTTCAACCTGGTGGGTCATTGCGGCTGAGCTGC |
| Ab2 scFv | GCTGCCAGTGGGTTCACCTTTTCAGATTACTACATGTCATGGGTCCGGCAGGCTCCCGGGAAGGGC |
| (VH-VL) | CTTGAGTGGGTGTCCTACATCTCAGGTTCTGGCGATGCAATCTACTATGCTGACTCTGTAAAGGGC |
| | CGCTTCACCATTAGCCGCGACAATTCAAAAAATACTCTGTACCTCCAGATGAACAGCCTTCGAGCC |
| | GAAGATACTGCCGTGTACTATTGTGCAAGGGATAGGTCTGATTGCGGTGGGGACGACAGATTCCTG |
| | TGTGACGGTTATTTTGACCTGTGGGGAAGAGGCACGCTTGTGTCCTTGTCTGGAGGAGGCGGATCA |
| | GGAGGCGGTGGAAGTGGCGGCGGAGGAAGCGACATACAGATGACACAGAGCCCGAGTTCCCTGTCC |
| | GCTAGTGTTGGGGACAGAGTCACCATTACATGCAGGGCCTCGCAGAGCACTAACAACTATGTGAAT |
| | TGGTATCAGCAAAAACCCGGCAAAGCACCAAAACTGCTGATATACGCGGCCTCCTCCCTCCAATCT |
| | GGTGTGCCTTCTCGTTTTAGCGGGAGTGGGTCGGGCACTGATTTCACGCTCACAATCAGTTCCTTG |
| | CAGCCCGAGGACTTTGCCACCTACTATTGTCAACAGAGCTATAGCATTCCTTTAACCTTCGGACCA |
| | GGGACAAAGGTCGATATCAAG (SEQ ID NO: 741) |
| TMPRSS4 | CAGGTTCAGCTCGTCCAGTCTGGGGCAGAAGTGAAAAAACCTGGAAGCAGCGTAAAAGTCTCCTGC |
| Ab3 scFv | AAGGCCTCCGGATATACGTTCACCTCTTATGACATTAACTGGGTGCGGCAGGCACCCGGCCAGGGC |
| (VH-VL) | CTTGAGTGGATGGGCGGGATTATCCCAATTTTTGGGACCACAAAGTTTGCTCAGAAGTTTCAGGGC |
| | CGCGTGACCATAACCGCTGATGAAAGTACAAGCACAGCTTACATGGAGCTCTCGAGTTTGAGGAGC |
| | GAGGATACTGCCGTGTACTATTGTGCCAGAGATTGGTATTCCTCGAGCTGGTATAACGGAGATCGT |
| | GGGGATTGGTTCGACCCATGGGGTCAGGGAACACTTGTAACTGTGTCCTCCGGCGGAGGAGGATCT |
| | GGTGGCGGTGGAAGTGGCGGTGGAGGCTCTGACATAGTTATGACTCAGTCACCACTGTCCCTCCCT |
| | GTGACTCCTGGCGAGCCCGCCTCAATCTCCTGTAGGAGTTCAGGGTCCCTGCTGCATTCTAATGGC |
| | TACAATTACCTGGACTGGTACCTGCAAAAGCCCGGGCAATCACCGCAGCTATTGATCTACGCGGCA |
| | AGTAGCTTACAGAGCGGCGTGCCTGACCGATTCTCTGGGTCAGGGAGCGGTACTGATTTCACCCTG |
| | AAGATCTCTAGAGTTGAAGCCGAGGACGTCGGTGTCTACTATTGCATGCAAGGGACCCACTGGCCC |
| | GGCACGTTTGGCCAAGGGACAAAAGTGGAAATCAAG (SEQ ID NO: 742) |
| TMPRSS4 | CAAGTGCAGCTCGTCCAGAGTGGTGCCGAAGTGAAAAAACCTGGGGCATCCGTAAAGGTGTCCTGC |
| Ab4 scFv | AAGGCCTCAGGCTATTTTTTCACTACATACTACTTACATTGGGTTCGGCAGGCTCCCGGGCAGGGC |
| (VH-VL) | CTGGAATGGATGGGCGTCATAAATCCGAATAGTCGCCTGACTAGCTACGCAGAATCCTTTCAGGGA |
| | AGGGTCACAATGACTAGAGATACCAGCACATCTACCGTCTATATGGAGCTGAGCAGTCTGCGTAGC |
| | GAGGACACCGCTGTGTACTATTGTGAGAGAGAAATGTTCCCATCGAGCTATGGGATTGATGTGTGG |
| | GGTCAGGGAACCACGGTGACAGTTTCAAGCGGAGGAGGAGGGTCTGGTTCCGGAGGAGGTGGATCT |
| | GGCGGCGGTGGATCAGACATTGTTATGACACAATCACCATTGAGCCTCCCAGTGACTCCAGGCGAG |
| | CCCGCCTCCATCTCGTGTCGAAGTTCCCAGTCCCTTCTGCACAGCAACGGCTATAACTACTTAGAC |
| | TGGTACTTGCAGAAGCCCGGCCAGAGTCCTCAGCTCCTAATCTACGCAGCGAGCACGCTCCAGTCA |
| | GGCGTGCCCGATAGGTTTTCTGGCAGCGGGTCTGGGACCGATTTCACGCTGAAGATTTCTCGGGTT |
| | GAAGCTGAGGACGTGGGCGTCTACTATTGCATGCAAGGGACCCACTGGCCTCCTACCTTCGGCCAA |
| | GGGACAAAACTTGAGATCAAG (SEQ ID NO: 743) |
| TMPRSS4 | CAAGTGCAACTGGTTCAGTCTGGGGCGGAAGTCAAGAAGCCAGGCTCTAGCGTAAAAGTCTCGTGC |
| Ab5 scFv | AAAGCCTCCGGGTACACTTTTACGAGCTATTACATGCATTGGGTGCGGCAGGCTCCCGGGCAGGGC |
| (VH-VL) | CTGGAATGGATGGGTCGGATCATTCCTATCCTGGGCGCCACCGATTATGCACAGAAATTCCAAGGG |
| | CGCGTAACTATAACTGCCGACGAGAGCACATCAACCGCCTACATGGAGCTCAGCTCCCTGCGATCC |
| | GAGGACACCGCTGTGTACTATTGCGCCAGGGCTGGCTATTCCTCCATCGCCGCGCGCCCGGCTTTT |
| | TGGGGTCAGGGAACTCTTGTTACCGTGTCTAGTGGAGGAGGCGGGTCCGGTTCAGGTGGAGGCGGA |
| | TCAGGCGGAGGAGGATCTGAGATTGTTATGACACAGTCGCCAGCAACCCTGAGTGTCAGCCCGGGC |
| | GAGAGGGCAACCCTCAGTTGTAGAGCTTCTCAGAGCGTGTCCTCCAACCTAGCATGGTATCAACAG |
| | AAGCCCGGCCAGGCACCCAGGTTGCTCATCTACGGCGCCTCAACAAGAGCCACCGGGATTCCTGCC |
| | CGTTTCTCTGGGAGCGGGAGCGGGACAGAGTTTACGCTTACAATAAGTAGTTTACAGTCAGAAGAT |
| | TTCGCTGTGTACTATTGTCAACAGTACTACTCTCCTTTCCCATTGACTTTTGGTGGCGGCACAAAG |
| | GTGGAAATCAAG (SEQ ID NO: 744) |
| TMPRSS4 | CAAGTGCAGCTCGTCCAGTCTGGAGCCGAAGTGAAAAAACCTGGGGCGTCTGTAAAAGTGTCCTGC |
| Ab6 scFv | AAGGCCTCCGGCTATACTTTCACGAGCTATTACATGCATTGGGTGCGGCAGGCACCAGGACAAGGG |
| (VH-VL) | TTGGAGTGGCTCGGGATAATCAACCCATCAGATTACACCACAAGCTACGCGCAGAAGTTTCAGGGC |
| | CGCGTCACCATGACCAGGGATACTTCTACAAGCACCGTCTACATGGAACTCTCGAGTCTAAGAAGT |
| | GAGGATACTGCTGTGTATTACTGCGCCCGAGTGGCCTCTTCATCCTGGTATCCAGGCGACGAGAAT |
| | TGGTATTTTGACCTGTGGGGCAGAGGCACACTTGTCACCGTTAGCTCAGGCGGTGGCGGGAGTGGA |
| | TCAGGCGGAGGTGGTAGTGGAGGCGGTGGATCGGACATTGTCATGACTCAGTCTCCCGATAGTCTT |
| | GCTGTGTCCCTGGGAGAAAGGGCAACAATTAATTGTAAGTCCTCCCAGTCTGTGCTGTACTCCAGC |
| | AACAACAAAAATTACTTGGCATGGTATCAGCAAAAGCCCGGTCAGCCGCCCAAACTGCTGATCTAC |
| | TGGGCTTCAACCCGGGAAAGCGGCGTGCCTGATCGTTTCAGCGGGAGCGGGTCTGGGACAGACTTT |
| | ACACTGACCATATCCAGCTTACAGGCTGAGGACGTTGCCATCTACTATTGTCAACAATACTATGCC |
| | ATTCCTTGGACTTTCGGGCAGGGAACGAAGGTTGAAATCAAG (SEQ ID NO: 745) |
| TMPRSS4 | CAGGTCCAGCTTGTCCAGTCCGGCGCCGAAGTCAAAAAACCCGGCGCGAGTGTCAAAGTGTCCTGC |
| Ab7 scFv | AAGGCCTCCGGCTATACTTTTTCCCGGTACTTCATGCATTGGGTGCGGCAGGCACCAGGCCAAGGG |
| (VH-VL) | TTGGAGTGGGTTGGCTGGATTAATCCCAACTCCGGGAATACCGGCTATGCCCAAAAGTTTCAGGGC |
| | CGCGTAACCATGACTCGAGATACATCTACATCCACTGTCTACATGGAGCTGTCTAGCCTGCGCAGC |
| | GAAGACACCGCAGTGTACTATTGCGCTAGGGTTGTTACCGGTGGCAGACTGGATGTGTGGGGACAA |
| | GGCACAACCGTGACAGTGTCAAGCGGAGGTGGAGGATCAGGAGGAGGCGGGAGTGGTGGAGGAGGT |
| | TCAGAAATTGTGATGACTCAGAGTCCTGCTACTCTGAGTGTGTCTCCTGGTGAGAGGGCAACCCTC |
| | AGTTGTAGAGCTTCGCAGAGAGTATCTAACAACTATCTCGCATGGTATCAGCAAAAGCCCGGGCAG |
| | GCTCCTAGGCTGCTTATCTACGGCGCGTCAACCCGGGCCTCTGGCATTCCGGCCCGTTTTTCTGGG |
| | AGCGGATCAGGGACTGAGTTCACACTAACAATATCCAGCTTACAGAGCGAGGACTTCGCCGTGTAC |
| | TATTGTCAACAGTACGGGAGCACACCATACACCTTCGGCCAAGGGACGAAGGTTGAAATCAAG |
| | (SEQ ID NO: 746) |
| TMPRSS4 | CAAGTACAGTTGGTCCAGAGCGGCGCAGAAGTCAAGAAGCCTGGGGCGTCAGTGAAAGTGTCCTGC |
| Ab8 scFv | AAAGCCTCCGGGTATCGGTTCACAAGCCAGTACATGCATTGGGTCCGGCAGGCGCCAGGCCAGGGT |
| (VH-VL) | TTGGAGTGGATGGGGATAATTAATCCTAGCGGCGGTAGTACGAGTTACGCGCAGAAGTTCCAGGGC |
| | CGCGTTACCATGACTCGTGACACCTCTACCAGCACCGTCTACATGGAACTCTCCTCCCTGAGGAGC |
| | GAGGATACCGCCGTGTATTACTGTGCTAGAGGCAGGATCGCTGTGGCAGGCCACCCACTCGGCTAC |
| | TGGGGTCAAGGGACTCTTGTAACAGTGTCCAGTGGAGGAGGAGGAAGCGGCGGTGGCGGTAGTGGC |
| | GGTGGAGGGTCAGACATTCAGATGACTCAGTCGCCCTCTTCACTGTCAGCTTCGGTTGGGGATAGA |
| | GTTACTATTACATGCCGAGCCTCTCAGAGTATCTCCTCCTGGCTGGCCTGGTATCAGCAAAAGCCG |
| | GGCAAAGCACCCAAGCTGCTGATCTACGGAGCCAGTTCCCTACAGAGCGGAGTGCCCTCTCGCTTC |
| | AGCGGGAGTGGCTCTGGGACCGACTTCACACTTACAATAAGCAGTTTACAGCCCGAAGATTTTGCA |
| | ACGTATTATTGTCAACAAGCTAACTCATTTCCACCTACCTTTGGCGGAGGAACAAAAGTGGAGATC |
| | AAG (SEQ ID NO: 747) |
| TMPRSS4 | CAGGTCCAACTGGTCCAGTCTGGCGCCGAAGTGAAAAAACCTGGAGCGTCCGTGAAGGTGTCCTGC |
| Ab9 scFv | AAGGCCTCCGGCTACACCTTCACCCGTTATTACATGCATTGGGTTCGCCAGGCACCCGGGCAAGGG |
| (VH-VL) | CTGGAATGGATGGGGTGGATTAATCCGAACTCCGGCGGTACAAATTATGCTCAGAAATTTCAGGGA |
| | AGGGTGACGATGACTCGGGATACTAGCACAAGTACAGTTTACATGGAGCTCTCGAGCCTACGATCA |
| | GAGGACACCGCCGTGTACTATTGTGCTAGAGGCGGCTCATGGGGCAGCGGGCCACTCGGCTATTGG |
| | GGACAAGGGACGCTTGTCACTGTATCCTCCGGAGGCGGTGGATCTGGAGGAGGCGGTAGTGGAGGC |
| | GGAGGGTCAGACATTCAGATGACACAGAGTCCCAGTTCCTTGTCGGCTTCAGTCGGTGATCGGGTG |
| | ACTATAACCTGTCAGGCCTCCCAGGATATTTCTAGGTTCTTGCATTGGTATCAGCAAAAGCCTGGG |
| | AAAGCACCCAAGCTGCTGATCTACGGTGCAAGCAACCTGAAATCTGGGGTGCCATCTAGATTCAGC |
| | GGCAGCGGGAGCGGGACCGACTTTACACTTACCATCTCAAGTTTACAACCAGAGGACTTCGCCACT |
| | TATTACTGCCAGCAGAGCTACTCTACACCTCCCACCTTTGGCGGTGGCACCAAGGTTGAAATCAAG |
| | (SEQ ID NO: 748) |
| TMPRSS4 | CAAGTGCAGCTTGTTCAGTCTGGAGCCGAGGTTAAAAAGCCCGGCGCGTCCGTTAAAGTGTCCTGC |
| Ab10 scFv | AAAGCCTCTGGCTACACCTTCACGAGCTACTACATGCATTGGGTCCGGCAGGCACCGGGCCAAGGG |
| (VH-VL) | TTGGAGTGGATGGGGATTATTAATCCATCTGGTGCCGGGACCACATATGGGCACAACTTTCAGGGA |
| | AGAGTCACAATGACTCGAGACACCTCGACAAGCACCGTCTACATGGAACTCTCTAGCCTGCGCTCA |
| | GAGGACACGGCTGTGTACTATTGTGCTCGCGGCCCTAGGGATACCGCAATGGTGCGGTTCGATTAT |
| | TGGGGTCAGGGAACACTTGTAACAGTGTCCAGTGGAGGCGGAGGGTCAGGTGGCGGAGGTTCAGGC |
| | GGAGGAGGCAGTGAAATTGTGATGACTCAGTCTCCTGCTACTCTGAGTGTATCACCTGGGGAAAGG |
| | GCAACTCTGAGTTGTAGAGCCTCGCAGAGCGTGTCCTCCTATCTGGCGTGGTACCAGCAGAAGCCC |
| | GGGCAGGCACCAAGGCTATTGATCTACGGAGCCAGCACTAGAGCCACCGGGATACCCGCCCGTTTT |
| | TCTGGCAGCGGGTCAGGCACTGAGTTCACCTTAACCATCTCCTCCCTGCAAAGTGAGGACTTCGCT |
| | GTCTATTACTGCCAGCAATATGGTAGCAGCCCAGGCACGTTTGGCCAGGGTACAAAGCTCGAAATC |
| | AAG (SEQ ID NO: 749) |
| TMPRSS4 | CAGGTCCAGCTTGTCCAGTCTGGGGCGGAAGTTAAAAAGCCTGGGGCTAGTGTAAAGGTGTCCTGC |
| Ab11 scFv | AAGGCCTCCGGCGGCACATTTAGCAATTACGCAATCAGTTGGGTGCGGCAGGCTCCAGGTCAAGGG |
| (VH-VL) | TTGGAGTGGGTGGGCCGGATTAATCCCAACTCCGGCGGCACTAATTATGCTCAGAAGTTTCAAGGG |
| | CGCGTTACCATGACCAGGGATACCAGCACTAGCACCGTCTACATGGAACTAAGCTCCCTGAGGTCT |
| | GAGGACACTGCTGTCTACTATTGTGCCAGAGGACGATACTCCTCTTCGTCCTGGGGTCAGGGAACC |
| | CTCGTGACCGTATCATCTGGCGGCGGAGGCTCAGGAGGAGGTGGAAGTGGTGGAGGAGGGTCAGAC |
| | ATTCAGATGACACAGAGTCCCTCATCACTGTCAGCCTCCGTTGGTGATAGAGTGACAATTACGTGC |
| | CGTGCCTCTCAGTCCATCAACAACTACTTAAACTGGTATCAGCAAAAACCGGGCAAAGCACCAAAA |
| | CTGCTGATCTACGCGGCAAGCAGTCTCCAGTCTGGCGTGCCTAGCCGCTTTAGCGGGTCTGGTAGC |
| | GGCACTGATTTCACACTGACAATAAGTAGCCTTCAGCCCGAGGACTTCGCCACCTATTACTGTCAA |
| | CAGTCGTATTCCACAAAATGGACTTTCGGGCAAGGGACGAAAGTGGAAATCAAG |
| | (SEQ ID NO: 750) |
| TMPRSS4 | CAGGTTCAGCTTGTCCAGTCTGGGGCAGAAGTCAAGAAGCCTGGCGCCTCCGTGAAAGTGTCGTGC |
| Ab12 scFv | AAGGCCTCCGGCTATACCTTCTCAAACTACTACATCCACTGGGTTCGACAGGCGCCCGGGCAGGGC |
| (VH-VL) | TTGGAATGGATGGGCTGGATTAATCCGAACTCCGGTGATACAAATTATGCACAAAAATTCCAAGGG |
| | CGGGTAACAATGACCCGGGATACCAGTACATCTACGGTCTACATGGAGCTAAGCAGCCTGCGCAGT |
| | GAAGATACCGCTGTGTACTATTGCGCTAGAGGCATGACCTGGAGGACCTCTGCTGCCACATACTGG |
| | GGTCAGGGAACACTTGTTACTGTGTCCTCCGGCGGAGGAGGATCAGGCGGTGGCGGAAGTGGAGGT |
| | GGAGGTTCAGACATTCAGATGACTCAAAGCCCAAGCTCACTGTCAGCTAGTGTGGGCGATAGAGTG |
| | ACGATTACATGTAGGGCCTCCCAAGGGATCTCGCGCTGGCTCGCGTGGTACCAGCAGAAGCCTGGG |
| | AAGGCACCCAAACTGCTGATCTACGGCGCCAGCAACCTCCAGACTGGGGTGCCCTCTCGTTTTTCT |
| | GGATCTGGTAGCGGCACTGACTTCACCCTGACAATATCCAGCTTACAGCCCGAGGACTTTGCCACC |
| | TATTATTGTCAACAGAGTTATAGCACTCCTCCAACTTTTGGGCCAGGGACGAAAGTCGACATCAAG |
| | (SEQ ID NO: 751) |
| TMPRSS4 | CAGGTTCAGTTGGTTCAGTCTGGGGCAGAAGTTAAAAAGCCAGGCGCGAGCGTGAAAGTGTCTTGT |
| Ab13 scFv | AAGGCCTCCGGCTATACCTTCACCAATTACTACATGCATTGGGTCCGGCAGGCTCCTGGGCAAGGG |
| (VH-VL) | TTGGAGTGGATGGGTTGGATAAGCGCCTACAACGGCAACACCAATTACGCTCAGAAACTGCAAGGG |
| | CGCGTGACGATGACAAGAGACACTTCAACTTCAACCGTCTATATGGAACTGAGTAGCCTCCGATCT |
| | GAGGACACCGCCGTGTACTATTGTGCAACAGCCAGCGGTTGGGGACACAGTAACTCCGCGGGCTAC |
| | TGGGGTCAAGGGACTCTTGTGACAGTCTCTTCAGGTGGCGGCGGATCTGGAGGTGGAGGGTCAGGC |
| | GGAGGCGGTAGTGAGATTGTCATGACACAGTCTCCCGCTACTCTGAGTGTGTCGCCCGGCGAAAGG |
| | GCAACCCTGAGTTGCAGAGCATCCCAGTCGGTGAACGGGAATTATCTCGCCTGGTATCAGCAAAAA |
| | CCGGGCCAGGCTCCCAGGCTGTTAATCTACGGAGTCTCCTCCCGGGCCAGCGGGATTCCTGCTCGT |
| | TTCAGCGGCTCCGGATCAGGCACTGAGTTCACACTAACCATAAGTAGCCTTCAGTCTGAAGATTTT |
| | GCCGTGTACTATTGTCAACAGTATGGAAGCTCTCCATACACGTTTGGGCAGGGAACAAAGGTTGAG |
| | ATCAAG (SEQ ID NO: 752) |
| TMPRSS4 | GAAGTCCAACTGCTCGAGAGCGGCGGTGGGCTGGTTCAGCCAGGCGGTAGTCTCCGGCTGTCCTGT |
| Ab14 scFv | GCTGCGAGTGGGTTCACCTTTTCCAATCACTATATGAGCTGGGTGCGGCAGGCTCCGGGAAAAGGC |
| (VH-VL) | CTGGAGTGGGTATCAGCCATCTCCGGGTCTGGCGGCAGCACTTATTACGCCGATAGCGTTAAGGGT |
| | CGCTTCACCATTAGCCGGGACAACTCCAAGAACACACTGTACTTGCAGATGAACTCATTACGCGCT |
| | GGGGATACCGCCGTGTACTATTGCGCGAGGGACCGATACAGATGGGGCAGAGGCTATTTTCAGCAT |
| | TGGGGACAAGGGACTCTAGTAACAGTGTCCTCGGGCGGAGGAGGGAGTGGAGGTGGTGGATCTGGA |
| | GGCGGTGGCTCTGACATCCAAATGACACAGTCACCTTCATCCTTGTCCGCCTCTGTCGGCGATAGA |
| | GTGACGATTACATGTAGGGCAAGTCAGAGGATTAGCACTTATCTGAATTGGTACCAGCAGAAGCCC |
| | GGAAAAGCACCCAAACTGCTGATCTACTCCGCATCGACCCTCCAGGCTGGGGTGCCATCTCGTTTT |
| | AGCGGGTCTGGCTCAGGCACCGATTTCACGCTTACAATAAGTAGCCTCCAGCCCGAGGACTTTGCC |
| | ACTTATTACTGCCAACAGGCCTACTCTCTTCCTTGGACCTTCGGGCAGGGAACTAAGTTAGAAATC |
| | AAG (SEQ ID NO: 753) |
| TMPRSS4 | CAGGTTCAGCTGGTCCAGTCGGGCGCCGAAGTGAAGAAGCCTGGGGCGAGTGTTAAAGTGTCCTGC |
| Ab15 scFv | AAGGCCTCTGGCTACACTTTCACTCGTTATTACATGCATTGGGTTCGGCAGGCACCTGGGCAAGGG |
| (VH-VL) | CTGGAATGGATGGGGTGGATTAACCCGAACTCCGGAGTGACCAATTTTGCTCAGAAGTTCCAAGGG |
| | CGCGTAACTATGACACGGGACACCAGTACATCAACCGTCTACATGGAGCTGTCCAGCCTAAGGAGC |
| | GAAGACACTGCTGTGTACTATTGTGCGAGGGTCAGAATTGGCTGGCTCCAGTCACCTCCACTGTAC |
| | TGGGGACAAGGAACGCTTGTGACAGTATCCAGCGGCGGCGGAGGATCTGGTGGTGGTGGATCTGGC |
| | GGCGGTGGATCAGACATACAGATGACCCAGTCGCCAAGCTCCTTGAGTGCTTCCGTCGGAGATAGA |
| | GTGACTATAACCTGCCGAGCCTCTCAGTCAATCAATACCTGGCTCGCCTGGTATCAGCAAAAGCCC |
| | GGCAAAGCTCCCAAACTGCTGATCTACGCAGCAAGCAGTCTCCAGAGCGGCGTGCCCTCACGCTTT |
| | TCTGGGTCTGGGAGTGGTACAGATTTCACCCTTACAATTAGCAGCTTGCAGCCAGAGGATTTTGCC |
| | ACGTATTATTGTCAACAGGCCATCTCCTTTCCCTTAACCTTCGGCGGCGGGACAAAAGTGGAGATC |
| | AAG (SEQ ID NO: 754) |
| TMPRSS4 | CAGGTCCAGCTCGTACAGTCCGGCGCGGAGGTTAAAAAGCCTGGTGCAAGTGTAAAAGTTTCCTGC |
| Ab16 scFv | AAGGCCTCCGGTTACACTTTCTCACGGCACTACATGCATTGGGTCCGGCAGGCACCCGGGCAGGGC |
| (VH-VL) | TTGGAATGGATGGGGCGCATTAATCCGAACTCCGGCGGTACTAATTACGCTCAGAAATTTCAAGGG |
| | CGAGTTACCATGACAAGGGATACCTCTACTTCCACCGTGTATATGGAACTGAGTAGCCTTAGGAGC |
| | GAGGACACCGCTGTGTACTATTGTGCCAGAAGCATCTACGGCGATTATTGGTTTGATCCTTGGGGA |
| | CAAGGGACACTTGTCACAGTGTCTAGTGGAGGCGGAGGGTCTGGAGGTGGAGGCAGCGGCGGAGGT |
| | GGATCTGACATTCAGATGACCCAGTCTCCATCATCACTGTCAGCTTCTGTGGGCGATAGAGTGACA |
| | ATAACATGCAGGGCCTCCCAGTCGATCAATCGCTGGCTCGCGTGGTATCAACAGAAGCCCGGGAAA |
| | GCACCAAAGCTGCTGATTTACGGCGCCAGCAACCTACAGAGCGGCGTGCCTAGTCGTTTTAGCGGT |
| | TCAGGGTCTGGAACCGACTTCACGTTGACAATAAGCAGTTTACAGCCCGAGGACTTTGCCACCTAT |
| | TATTGTCAACAGGCTAACTCCTTCCCATACACTTTCGGGCAAGGCACTAAGCTGGAAATCAAG |
| | (SEQ ID NO: 755) |
| TMPRSS4 | CAAGTGCAGCTTGTTCAGTCCGGCGCTGAAGTGAAAAAACCCGGCGCATCAGTTAAAGTGTCCTGC |
| Ab17 scFv | AAGGCCTCCGGCTATACTTTCACATCGTATTACATCCACTGGGTCCGGCAGGCTCCTGGGCAGGGA |
| (VH-VL) | CTAGAGTGGATGGGGTGGATGAGCCCTAATAGCGGCGATACCGGTTACGCCCAAAAGTTTCAGGGC |
| | CGCGTAACCATGACCAGGGACACATCTACATCCACCGTCTACATGGAGCTTAGCTCTTTGCGATCC |
| | GAAGATACGGCTGTGTACTATTGCGCCCGGCTCGTCCGTGGCGGGTTTGATTACTGGGGTCAGGGA |
| | ACGCTGGTAACAGTGTCAAGTGGTGGCGGAGGATCAGGCGGAGGCGGATCTGGTGGTGGCGGCTCT |
| | GACATTCAGATGACCCAGTCTCCAAGCAGCCTGAGTGCCTCGGTTGGGGACAGAGTGACTATTACC |
| | TGTCGCGCTAGTCAGGGAATCTCCTCCTACCTGAACTGGTATCAACAGAAGCCCGGGAAGGCACCC |
| | AAACTCTTGATTTACGCGGCGAGTAGACTCCAAAGCGGAGTGCCGAGCAGGTTTTCCGGGAGCGGG |
| | TCTGGCACCGATTTCACACTGACAATAAGTAGTTTACAGCCAGAGGACTTCGCAACTTACTATTGT |
| | CAACAGTCTTATAGAAGCCCTCCAACTTTCGGGCAGGGAACTAAGCTGGAAATCAAG |
| | (SEQ ID NO: 756) |
| TMPRSS4 | CAGGTCCAGCTCGTACAGAGTGGCGCCGAAGTCAAGAAGCCTGGAGCTTCCGTCAAAGTGTCCTGC |
| Ab18 scFv | AAAGCCAGCGGCTATACTTTCACGTCCTCAGGAATTAATTGGGTTCGGCAGGCACCCGGGCAGGGC |
| (VH-VL) | TTGGAGTGGATGGGGTGGATTAATCCCAACTCCGGCGGCGCAAAATATGCCCAACGCTTCCAGGGA |
| | CGGGTTACTATGACCCGAGATACCAGCACCTCTACTGTCTACATGGAGCTGAGCAGCCTTAGGAGT |
| | GAAGATACCGCTGTGTACTATTGCGCCAGGGCGAGAGGGTATTCTGGCTCGAAAAGAGATTTTCAG |
| | CATTGGGGACAAGGGACATTGGTTACAGTCTCATCAGGAGGAGGCGGTTCTGGCGGCGGCGGGAGT |
| | GGAGGTGGTGGATCTGACATTGTGATGACACAGAGTCCGGACAGCCTTGCTGTGTCTCTCGGTGAA |
| | CGCGCGACCATCAACTGCAAGTCCTCCCAGTCGGTGCTGTACTCAAGCAACAACAAGAATTACCTG |
| | GCCTGGTATCAACAGAAACCGGGCCAGCCTCCAAAGCTGCTGATCTACTGGGCTAGTACCAGGGAG |
| | TCTGGGGTGCCTGATCGTTTCTCCGGTAGCGGCTCCGGGACAGACTTCACCCTAACTATAAGCAGT |
| | TTACAAGCAGAGGACGTGGCCGTATATCACTGTCAACAGTACTACAATACACCATTTACGTTTGGA |
| | CCAGGGACAAAAGTGGACATCAAG (SEQ ID NO: 757) |
| TMPRSS4 | CAAGTGCAGTTGGTTCAGTCTGGAGCCGAAGTGAAGAAGCCTGGGGCGAGCGTCAAAGTGTCCTGC |
| Ab19 scFv | AAGGCCTCCGGCTACACTTTTAACCGGAAATTCATGCATTGGGTTCGGCAGGCACCCGGTCAAGGG |
| (VH-VL) | CTGGAATGGATGGGCTGGATGAACCCGAACAACGGTGCAACTAATTATGCACAGAAGTTCCAGGGC |
| | CGCGTGACAATGACACGAGATACCAGCACTTCAACCGTCTACATGGAGCTCTCTTCCCTAAGGAGC |
| | GAAGATACCGCCGTCTACTATTGTGCTAGAGGAAGAGGCTATTATGGTTCTGGTTCATACTATGGG |
| | GACTATTGGGGACAAGGGACCCTTGTAACAGTCTCCTCTGGAGGAGGCGGATCAGGCGGTGGCGGA |
| | AGCGGTGGAGGAGGCTCAGATATTCAGATGACCCAGTCGCCAAGTTCCTTGTCCGCTTCCGTAGGC |
| | GACCGCGTGACTATAACTTGCAGGGCCTCTCAAAGTATCTCGAGATACCTGAATTGGTACCAGCAG |
| | AAGCCAGGGAAAGCTCCAAAACTGCTGATCTACGGAGCGAGCAATCTCCAGAGCGGCGTGCCGAGT |
| | CGTTTTAGCGGCAGCGGGTCTGGGACAGACTTCACGCTGACAATAAGTAGCTTACAGCCCGAGGAT |
| | TTTGCTACGTATTACTGTCAACAGAGTTACTCCACACCTCCCACCTTCGGCCCTGGGACGAAAGTG |
| | GACATCAAG (SEQ ID NO: 758) |
| TMPRSS4 | CAAGTGCAGCTGGTGCAGAGTGGCGCGGAGGTCAAAAAGCCTGGGGCGTCCGTGAAAGTGTCCTGC |
| Ab20 scFv | AAGGCCTCCGGATACACCTTCACCCGATATTACATGCATTGGGTTCGGCAGGCACCAGGCCAAGGG |
| (VH-VL) | CTCGAATGGATGGGGTGGATGAATCCCAACTCCGGGAATGCAGGCTATGCTCAGAAACTGCAAGGC |
| | CGCGTGACTATGACTCGCGACACCTCCACCTCAACTGTCTACATGGAGCTAAGTAGCTTGAGGTCT |
| | GAGGACACCGCTGTGTATTACTGTGCCAGAGGCTACAACTGGTTTGATCCATGGGGTCAGGGAACA |
| | CTTGTAACAGTGTCAAGTGGAGGAGGTGGAAGTGGCGGCGGAGGCTCAGGTGGTGGCGGAAGCGAC |
| | ATTCAGATGACCCAGTCGCCCTCTTCACTGTCCGCCTCAGTTGGGGACAGAGTAACCATTACCTGT |
| | AGGGCCTCTCAGAATATCGCCACATACCTGAGCTGGTATCAGCAAAAGCCTGGCAAAGCTCCAAAG |
| | CTGCTGATCTACGGTGCAAGCGCCCTCCGGTCTGGAGTCCCTAGCCGTTTTTCTGGGTCCGGGAGC |
| | GGGACAGATTTCACTCTCACAATATCTAGCTTACAGCCGGAAGATTTCGCTACTTATTACTGCCTT |
| | CAGCACAACACATATCCCTTGACATTTGGCGGAGGCACGAAGGTTGAAATCAAG |
| | (SEQ ID NO: 759) |
| TMPRSS4 | CAGGTTCAACTGGTCCAGTCAGGCGCTGAAGTGAAGAAGCCTGGCGCCTCTGTGAAAGTGTCCTGC |
| Ab21 scFv | AAAGCCTCCGGTTATAGTTTCTCTGGCTACTATCTGCATTGGGTTAGGCAGGCACCTGGGCAGGGC |
| (VH-VL) | CTGGAATGGATGGGGTGGATGAATCCCGATTCTGGGAATACTGGCTACGCGCAAAACTTTCAGGGA |
| | CGGGTTACAATGACCCGAGATACTTCCACCTCAACCGTCTACATGGAACTCTCCTCTCTGCGGAGC |
| | GAGGACACCGCCGTGTACTATTGTGCTCGCCTGCACCGTGGCGGGCACGATTACTGGGGTCAGGGA |
| | ACACTTGTCACTGTATCGAGTGGTGGCGGAGGGTCCGGAGGTGGAGGCTCGGGTGGAGGTGGATCA |
| | GACATTCAGATGACTCAGAGTCCAAGCAGTTTGAGTGCTTCTGTGGGCGACAGAGTAACTATTACA |
| | TGCAGGGCCTCCCAGTCAATCTCTCGCTATCTCAACTGGTATCAGCAAAAACCCGGCAAGGCACCG |
| | AAGTTACTGATCTACGCAGCGAGCACGCTACAGAGCGGAGTGCCATCAAGATTTAGCGGCAGCGGG |
| | TCCGGGACCGATTTCACCCTTACGATAAGCAGCTTGCAACCTGAGGACTTCGCCACCTACTATTGT |
| | CAACAGTCTTACTCCACACCCGTGACATTTGGGCAGGGAACACGGCTCGAGATCAAG |
| | (SEQ ID NO: 760) |
| TMPRSS4 | CAGGTTCAGCTGGTCCAGTCTGGCGCCGAAGTTAAAAAACCTGGCGCGAGCGTGAAAGTGTCCTGC |
| Ab22 scFv | AAGGCCTCCGGTTACACATTCACTTCATATTACATGCATTGGGTGCGGCAGGCTCCTGGGCAAGGG |
| (VH-VL) | TTGGAGTGGATGGGGCGGATTAATCCGCACTCTGGCGACGCCGATTTCGTCGATAAGTTTCAAGGG |
| | CGCGTGACTATGACCAGGGATACTAGCACCAGCACCGTCTACATGGAGCTATCAAGTTTGCGCTCC |
| | GAAGATACCGCAGTGTACTATTGCGCCAGGGACAGAAGAGGATATGGCGGCAATAGCCTTGACTAT |
| | TGGGGACAAGGGACACTTGTAACTGTTAGTTCTGGCGGTGGAGGAAGCGGAGGAGGCGGTTCCGGA |
| | GGAGGCGGTTCAGACATTCAGATGACTCAGAGTCCAAGTTCACTGTCAGCTTCCGTGGGTGACAGA |
| | GTGACGATAACATGTAGGGCCGGGCAGAACATCAAGCGATACCTCAACTGGTACCAGCAGAAACCT |
| | GGGAAGGCACCCAAACTGCTGATATATGCAGCGAGCAGCCTCCAGTCTGGGGTACCCTCTCGTTTC |
| | AGCGGCTCTGGGTCCGGCACCGATTTTACGCTCACAATCAGTTCCCTGCAACCAGAGGACTTTGCT |
| | ACATACTATTGTCAACAGTCGTACAGCTCGCCCTTAACCTTCGGAGGCGGCACCAAAGTCGAAATC |
| | AAG (SEQ ID NO: 761) |
| TMPRSS4 | CAGGTTCAGTTGGTACAATCAGGAGCAGAAGTGAAAAAGCCTGGAGCCTCAGTGAAGGTGTCCTGC |
| Ab23 scFv | AAAGCCTCCGGCTATACATTCACCCGGAATTACCTCCATTGGGTTCGGCAGGCTCCTGGCCAGGGC |
| (VH-VL) | CTGGAGTGGATGGGGATTATTAATCCCAGTGGCGGCAGCACCACTTACGCCCAGAAGTTCCAAGGC |
| | CGCGTAACAATGACCCGAGATACATCAACTTCCACCGTCTACATGGAACTCTCCAGCCTGAGGAGC |
| | GAGGACACGGCTGTGTACTATTGTGCCCGCGGCAGGACATGGTTCAGATCAGGCATGGATGTGTGG |
| | GGTCAAGGGACCACCGTTACAGTGTCCTCCGGAGGAGGAGGGAGCGGTGGCGGAGGAAGTGGTGGT |
| | GGAGGATCAGAAATCGTGATGACTCAGAGTCCCGCCACACTGAGTGTCAGCCCGGGCGAGCGGGCA |
| | ACACTGAGTTGCAGAGCCTCCCAGTCTGTCGGCAACTATCTAGCTTGGTATCAGCAAAAGCCTGGG |
| | CAGGCTCCAAGGCTGCTTATTTACGGCGCGAGCACTAGAGCAACTGGGATACCAGCGCGTTTTTCT |
| | GGGTCTGGGTCTGGGACTGAGTTTACCCTTACCATCAGCTCGTTACAGTCTGAGGACTTCGCAGTG |
| | TACTATTGTCAACAGTACCACTCTAGCCCACCATATACGTTTGGGCAGGGTACGAAAGTCGAAATC |
| | AAG (SEQ ID NO: 762) |
| TMPRSS4 | CAAGTGCAGCTGGTCCAGTCAGGCGCGGAAGTCAAAAAATCTGGCGCGAGCGTTAAGGTGTCCTGC |
| Ab24 scFv | AAGGCCTCCGGTTACACCTTTACGTCCTATTACATGCATTGGGTCCGGCAGGCACCTGGGCAGGGC |
| (VH-VL) | CTGGAGTGGATGGGCGTTATAAATCCCTCTGGCGGCACTACAAGCTACGCCCAGAAGTTTCAAGGG |
| | CGGGTGACAATGACTCGAGAGACAAGCACTAGCACCGTCTACATGGAACTCAGCTCGCTGCGCTCA |
| | GAAGATACCGCCGTGTATTACTGCGCAAGAGGAAGAGGGTGGCTGAGGTCTGCTCTCGGCTATTGG |
| | GGTCAGGGTACACTTGTAACCGTGTCCTCCGGAGGCGGAGGTAGTGGCGGAGGAGGATCTGGCGGA |
| | GGAGGGTCTGACATTCAGATGACCCAGTCTCCCTCGTCCTTGTCAGCCAGCGTGGGTGATCGCGTT |
| | ACCATTACCTGTAGGGCTAGTCAGAGTATCTCCTCCTGGCTCGCTTGGTATCAGCAAAAGCCCGGC |
| | AAAGCTCCTAAGCTGCTGATCTACGCAGCCTCAACTTTGCAGTCCGGCGTACCGAGTCGTTTTTCT |
| | GGGAGCGGGAGCGGGACCGACTTCACGCTAACTATTAGTAGCTTACAGCCAGAGGACTTCGCCACT |
| | TATTATTGTCAACAGTCATACAACACGCCATACACATTCGGGCAAGGCACAAAACTTGAGATCAAG |
| | (SEQ ID NO: 763) |
| TMPRSS4 | CAGGTCCAGTTAGTGCAGTCTGGGGCGGAGGTGAAAAAGCCAGGAGCGAGCGTGAAAGTGTCCTGC |
| Ab25 scFv | AAAGCCTCCGGCGGGCGATTTTCAACATACGCCCTGTCATGGGTTCGGCAAGCACCCGGCCAGGGC |
| (VH-VL) | CTGGAGTGGATGGGGTGGATTAACCCGAACTCCGGCGGCACAAATTACGCCCAGAAATTCCAGGGC |
| | CGCGTAACTATGACTCGAGACACCTCAACTTCCACCGTCTACATGGAGCTAAGCTCTCTGAGGAGT |
| | GAAGACACAGCAGTGTACTATTGCGCCAAGTCCTTGTGGTGGAGTCCAAGCCACTACTATTACTAT |
| | GGGATGGATGTGTGGGGTCAAGGCACCACAGTCACAGTTTCCAGCGGAGGAGGTGGAAGTGGAGGC |
| | GGCGGCTCTGGAGGAGGAGGCAGTGAAATCGTGATGACACAGAGCCCTGCTACCCTGAGTGTTAGC |
| | CCTGGGGAAAGGGCTACCCTCTCGTGTAGAGCCTCACAATCTGTCTCCTCCAATTATCTCGCCTGG |
| | TATCAACAGAAGCCCGGGCAGGCTCCCAGGTTGCTTATCTACGGGATTTCAACTAGAGCATCGGGT |
| | ATTCCCGCTAGATTTTCTGGTTCTGGCAGCGGGACCGAGTTTACCCTTACGATAAGTAGCCTCCAG |
| | AGCGAGGATTTCGCAGTGTACTATTGTCAACAGCGCTCTAACTGGCCACCTAGCATAACGTTCGGG |
| | CAGGGAACTCGTCTGGAAATCAAG (SEQ ID NO: 764) |
| TMPRSS4 | GAGGTTCAACTGCTCGAGTCTGGCGGCGGGCTTGTTCAGCCCGGCGGTTCTTTGAGGTTGTCATGT |
| Ab26 scFv | GCCGCGTCTGGGTTTACCTTCTCCTCCTATGCCATGCATTGGGTGCGGCAGGCTCCTGGCAAGGGC |
| (VH-VL) | CTGGAGTGGGTGGCTGTCATCTGGTACGACGGGAGCAGCAAATACTACGCCGATTCCGTGAAAGGC |
| | CGCTTCACCATTTCTCGAGACAACTCAAAGAATACGCTGTACCTCCAGATGAACTCCCTGCGCGCA |
| | GAAGATACCGCTGTGTACTATTGCGCGCGTGGGGAAGTGCGGAGAGGGTTCCAGCACTGGGGTCAG |
| | GGAACACTTGTCACAGTGTCCTCCGGAGGAGGCGGGTCAGGTGGAGGTGGTAGTGGAGGAGGAGGC |
| | AGTGACATTCAGATGACACAGTCTCCTAGTTCACTGTCAGCCAGCGTCGGTGATAGAGTTACAATA |
| | ACTTGTAGGGCCTCGCAGAATGTAGGAAGCTGGCTCGCATGGTATCAACAGAAGCCAGGGAAAGCT |
| | CCCAAGCTGCTGATCTACGCAGCAAGCAGCCTCCAGAGCGGCGTACCATCGAGGTTTTCCGGCAGC |
| | GGAAGTGGCACCGACTTCACCTTAACTATTAGATCTCTACAGCCGGAAGATTTTGCCACCTATTAC |
| | TGCCAACAGAGTTATTCCACTCCCATCACGTTTGGGCAAGGCACTCGGTTAGAGATCAAG |
| | (SEQ ID NO: 765) |
| TMPRSS4 | CAGGTTCAGCTGGTCCAGTCTGGCGCCGAAGTGAAGAAGCCCGGCGCATCCGTCAAAGTGTCGTGC |
| Ab27 scFv | AAGGCCTCCGGATATACATTTTCCCGCTACTACATGCATTGGGTTCGGCAGGCTCCTGGCCAAGGG |
| (VH-VL) | TTGGAATGGATGGGGTGGATGAATCCGAACTCCGGCGATACTGGTTATGCACAGAAATTCCAAGGG |
| | AGGGTAACAATGACCCGCGACACCAGCACATCTACGGTCTACATGGAACTCTCCTCACTGCGAAGC |
| | GAGGATACTGCCGTGTATTACTGCGCTAAGGGAAGGGAGTGGCTGAGATCTCCTTTCGACTATTGG |
| | GGTCAAGGGACTCTTGTGACCGTTAGCAGCGGTGGCGGCGGTAGTGGAGGTGGAGGAAGCGGCGGA |
| | GGAGGATCAGACATTCAGATGACACAGAGCCCTTCTTCACTGTCAGCTTCTGTGGGCGATAGAGTG |
| | ACTATTACCTGTCGTGCCAGTCAGTCAATCAGCACTTGGCTCGCTTGGTATCAGCAAAAGCCAGGG |
| | AAAGCGCCCAAACTGCTGATCTACGCAGCGAGTAGTCTCCAGTCTGGCGTGCCCTCTCGCTTTTCG |
| | GGCTCCGGGTCCGGCACCGATTTCACCCTTACAATTTCAAGCCTACAGCCAGAGGACTTTGCCACC |
| | TATTACTGTCAACAGTTAAGCTCCTACCCATTGACATTCGGGCAAGGGACAAAAGTAGAGATCAAG |
| | (SEQ ID NO: 766) |
| TMPRSS4 | CAGGTTCAACTGGTACAGAGCGGCGCCGAAGTGAAAAAACCCGGCGCGAGCGTGAAAGTGTCCTGC |
| Ab28 scFv | AAGGCCTCCGGCTACACCTTCACTGGATATTACATGCATTGGGTCCGGCAGGCTCCCGGGCAGGGC |
| (VH-VL) | CTGGAGTGGATGGGATGGATGAATCCAAACTCCGGAAATACCGGTTACGCCCAGAAATTCCAGGGA |
| | CGAGTTACAATGACTCGTGATACATCCACTAGCACCGTCTACATGGAACTCTCTTCTCTACGCTCC |
| | GAGGACACCGCAGTGTACTATTGCGCTAGGCTCAGAGCGAAGGGCGGCGGTTTTGACTATTGGGGT |
| | CAGGGTACACTTGTTACTGTGTCAAGTGGCGGAGGAGGGTCTGGAGGAGGCGGTAGTGGCGGTGGC |
| | GGTTCTGACATCCAGATGACCCAGTCACCCTCTTCACTGTCAGCCAGCGTAGGCGATAGAGTCACT |
| | ATTACCTGTAGGGCTTCGCAAGGGATTGGAAACTATTTGGCTTGGTATCAACAGAAGCCCGGGAAG |
| | GCACCTAAACTGCTGATCTACGCAGCAAGCAGCTTAGAAAGCGGAGTGCCGAGTCGCTTTAGCGGG |
| | TCCGGGTCTGGCACAGATTTCACGCTTACAATAAGTAGTTTGCAGCCAGAGGATTTTGCCACGTAC |
| | TATTGTCAACAGGGCTACCGGTTCCCACCTACCTTTGGGCCTGGGACAAAGGTGGACATCAAG |
| | (SEQ ID NO: 767) |
| TMPRSS4 | CAGGTTCAGCTCGTACAATCTGGGGCAGAAGTGAAAAAGCCCGGCGCCAGCGTTAAGGTGTCCTGC |
| Ab29 scFv | AAAGCCAGCGGCTATACCTTTGCAAATTACAACATCCACTGGGTGCGGCAAGCACCTGGGCAGGGC |
| (VH-VL) | CTGGAGTGGATGGGGTGGATGAATCCAAACTCCGGCAATACCGGTTACGCTCAGAAATTTCAAGGA |
| | CGGGTGACCATGACCAGGGACACTAGCACATCAACTGTGTACATGGAGCTAAGCAGTCTGCGAAGC |
| | GAAGATACCGCTGTCTACTATTGCGCTCGCCCACGATATTCATCTGGGTGGTATGGGTGGTATTTT |
| | GACCTGTGGGGCAGAGGTACACTTGTAACAGTGTCCAGTGGAGGCGGAGGATCCGGTGGTGGCGGA |
| | AGTGGAGGCGGTGGTTCTGACATTGTCATGACGCAGAGTCCGTTGTCCCTGCCCGTGACACCTGGG |
| | GAACCAGCGTCCATATCTTGTAGATCATCACAGTCCCTCCTCCATTCGAACGGCTACAACTACTTA |
| | GATTGGTACTTGCAGAAGCCCGGGCAATCGCCTCAGCTCCTGATCTACTTAGGATCTAATAGGGCC |
| | TCTGGAGTGCCAGATCGCTTCTCAGGCAGCGGGAGCGGCACAGATTTCACGCTGAAAATTAGTCGT |
| | GTTGAGGCCGAAGACGTAGGCGTCTACTATTGTATGCAGAGCACTTATTGGCCTCCAACTTTCGGG |
| | CAGGGAACAAAGCTTGAGATCAAG (SEQ ID NO: 768) |
| TMPRSS4 | CAGGTTCAACTGGTCCAGTCGGGTGCCGAGGTGAAAAAGCCCGGAGCGTCAGTGAAAGTGTCCTGC |
| Ab30 scFv | AAGGCTTCTGGCTATACATTCACGACTTATTACATGCATTGGGTGCGGCAGGCACCAGGCCAAGGG |
| (VH-VL) | CTAGAATGGATGGGGTGGATGAATCCGAACTCCGGAAACACAGGCTACGCCCAGAAGTTCCAAGGG |
| | CGGGTAACAATGACCCGAGATACCAGCACCAGCACGGTCTACATGGAGCTCTCCTCACTGCGCTCC |
| | GAAGATACCGCAGTGTACTATTGTGCCCGCGCTAGGACCTGGCTGCTGTCTCCATTTGACTATTGG |
| | GGACAAGGGACTCTTGTGACCGTTAGTAGCGGTGGAGGAGGATCTGGTGGCGGCGGGTCCGGCGGT |
| | GGAGGTTCAGAAATTGTTATGACTCAGAGTCCTGCCACCCTGAGTGTAAGTCCTGGGGAGAGGGCA |
| | ACTCTGAGTTGTAGAGCCTCCCAGTCTGTGGGCAGATATTTGGCCTGGTACCAGCAGAAGCCTGGG |
| | CAGGCTCCCAGGCTCCTCATCTACGGCGCGTCCACCAGAGCAACCGGGATTCCAGCTCGTTTTTCA |
| | GGCAGCGGCTCTGGCACAGAGTTCACGTTGACTATAAGCAGCTTACAGTCTGAGGACTTCGCCGTC |
| | TATTACTGCCAGCACTATGACAGCTCACCCATGTACACTTTTGGGCAGGGAACAAAACTTGAAATC |
| | AAG (SEQ ID NO: 769) |
| TMPRSS4 | CAGGTCCAGCTGGTGCAGTCTGGGGCAGAAGTGAAAAAACCCGGAAGCAGCGTAAAGGTGTCCTGC |
| Ab31 scFv | AAGGCCTCCGGTTACACCTTCCGCGGGTCTGGCATCTCCTGGGTCCGGCAAGCTCCTGGGCAAGGG |
| (VH-VL) | TTGGAGTGGATGGGGATTATCTACCCAGCTGACAGCGAGACACGCTACAGCCCGAGTTTTCAGGGC |
| | CGAGTGACCATTACCGCGGATGAGTCTACTTCAACTGCCTATATGGAACTCTCTAGTCTCCGGTCC |
| | GAGGACACTGCGGTGTACTATTGTGCAAGGGAGAGTTCCTCCTGGGATTATTTCGATTATTGGGGA |
| | CAAGGAACACTTGTAACAGTGTCAAGCGGCGGCGGAGGATCTGGAGGCGGAGGTTCTGGAGGTGGA |
| | GGTTCAGAAATAGTTATGACTCAGTCGCCTGCCACACTGAGTGTCTCGCCCGGCGAAAGGGCTACA |
| | CTGTCTTGCAGAGCCTCCCAAAGCGTTAGATCATACCTGGCATGGTATCAGCAAAAACCTGGCCAG |
| | GCTCCAAGGCTGCTCATATACGGCGCCTCAACCAGAGCCACCGGCATTCCAGCCCGTTTTTCAGGG |
| | AGTGGTAGCGGGACCGAGTTCACTCTTACGATCAGCTCCCTACAGAGCGAGGACTTCGCAGTGTAT |
| | TACTGTCAACAGCACGGCAGTTTGCCCTTAACCTTTGGTCAAGGGACGAAAGTCGAAATCAAG |
| | (SEQ ID NO: 770) |
| TMPRSS4 | CAGGTTCAGTTGGTGCAGAGCGGCGCGGAGGTCAAAAAGCCTGGGAGCAGCGTAAAAGTGTCCTGT |
| Ab32 scFv | AAGGCCTCCGGCGGCACATTCTCCTCATACGCTATATCCTGGGTCCGGCAGGCTCCTGGGCAGGGC |
| (VH-VL) | TTGGAATGGATGGGTCGGATTAATCCTAGCGGCGGGAGCACTTCTTATGCCCAAAAGTTTCAAGGG |
| | CGCGTGACCATGACTCGAGATACCAGTACAAGCACCGTCTACATGGAACTTAGTTCACTGAGGTCT |
| | GAGGACACCGCAGTGTACTATTGTGCTAGAGGCCGATACAGTTCGTCGTCTTGGGGTCAGGGTACG |
| | CTTGTGACTGTGTCCTCCGGAGGAGGAGGAAGTGGAGGAGGAGGAAGCGGCGGCGGTGGAAGCGAC |
| | ATTCAGATGACCCAATCTCCATCTTCACTGTCAGCCTCAGTTGGGGATAGAGTCACCATTACATGC |
| | AGGGCCTCTCAAAGCATCTCAACCTACCTGAACTGGTATCAGCAAAAACCCGGGAAAGCACCCAAA |
| | CTGCTCATCTACGCTGCCTCCTCCCTCCAGCGCGGCGTGCCGAGTCGTTTCTCTGGGTCAGGGAGC |
| | GGTACAGACTTTACGCTGACTATCTCCTCCCTACAGCCAGAGGATTTTGCAACTTATTACTGCCAG |
| | CAGTCTTATACTACACCCTTAACCTTCGGCGGCGGCACAAAGGTTGAAATCAAG |
| | (SEQ ID NO: 771) |
| TMPRSS4 | CAGGTCCAGCTTGTCCAGAGCGGCGCCGAAGTTAAGAAGCCTGGGGCGTCCGTAAAAGTGTCCTGC |
| Ab33 scFv | AAGGCCTCCGGATATACTTTTTCTAACTACTACATGCATTGGGTGCGACAGGCACCCGGCCAGGGT |
| (VH-VL) | CTGGAGTGGGTGGGCTGGATGAACCCGAAAAGCGGGAATACTGGTTATGCACAGAAATTTCAAGGG |
| | CGGGTGACAATGACCAGGGACACGAGCACGAGCACAGTTTACATGGAACTCAGTTCACTGCGCTCG |
| | GAGGATACCGCCGTCTACTATTGTGCCAGAGGCCGGACCTGGATTCAGTCCTCGCTGGGTTATTGG |
| | GGTCAGGGAACTCTTGTGACAGTGTCCAGTGGTGGCGGAGGATCAGGCGGAGGAGGGTCTGGAGGC |
| | GGAGGCTCAGACATTCAAATGACCCAGTCACCCAGCTCATTGTCCGCCTCTGTAGGCGATAGAGTG |
| | ACTATTACTTGTAGGGCTTCTCAGTACATCTCTCGCTGGTTGGCTTGGTATCAGCAAAAGCCAGGG |
| | AAAGCACCAAAACTCCTGATCTATGGGAGTTCTACCCTACAATCCGGCGTTCCATCTCGTTTCTCC |
| | GGGAGTGGCAGCGGAACCGATTTCACCCTCACGATAAGCAGCTTACAGCCCGAGGACTTCGCTACA |
| | TATTACTGCCAACAGTACTACAGTACACCTTTCACCTTTGGCCCTGGGACAAAGCTGGAAATCAAG |
| | (SEQ ID NO: 772) |
| TMPRSS4 | CAGGTCCAGCTCGTCCAGAGCGGCGCCGAAGTGAAAAAACCCGGAGCTTCAGTGAAAGTTTCCTGC |
| Ab34 scFv | AAAGCCTCCGGTTATACCTTCACTGGTTACTACATTCACTGGGTCCGGCAGGCTCCAGGCCAAGGG |
| (VH-VL) | TTGGAGTGGATGGGGTGGATGAATCCACATAGTGGCAACACCGGATACGCCCAGAAATTCCAAGGG |
| | CGGGTTACAATGACACGTGATACCTCTACTTCTACTGTGTATATGGAACTCAGCTCGCTGCGCTCT |
| | GAGGATACCGCTGTGTACTATTGCGCCAGGGAGGGCGGTCGATACAGTTCTGGCAGACTGGGGTAT |
| | TGGGGACAAGGGACACTTGTCACAGTATCCAGCGGAGGAGGAGGATCAGGCGGAGGAGGCTCGGGA |
| | GGCGGTGGTTCAGACATTCAGATGACTCAGAGTCCCAGCTCACTGTCAGCTTCTGTGGGCGACAGA |
| | GTGACCATTACATGCCGCGCAAGTCAAGGGATCTCCTCCTGGTTGGCGTGGTATCAACAGAAGCCC |
| | GGCAAGGCACCTAAGCTGCTGATCTACGCAGCCAGCACCCTACAGACTGGGGTGCCTTCTAGGTTT |
| | AGCGGTAGTGGCAGCGGGACTGACTTCACGCTTACGATCAGCTCCTTACAGCCGGAAGATTTTGCC |
| | ACCTACTATTGTCAACAGTCCAAATCCATACCTATAACCTTTGGCGGCGGGACAAAGGTTGAGATC |
| | AAG (SEQ ID NO: 773) |
| TMPRSS4 | CAAGTGCAACTGGTGCAGAGCGGCGCTGAGGTTAAAAAGCCGGGCGCCAGCGTCAAAGTGTCCTGC |
| Ab35 scFv | AAGGCCTCCGGTTACACGTTCACCGGCTACTACATGCATTGGGTCCGGCAGGCACCAGGCCAGGGC |
| (VH-VL) | CTGGAATGGATGGGGAAAATCTCAGCCCACTCTGGGGAAACCAAGTATGCTCAGAACGTGCAAGGC |
| | CGAGTCACAATGACTAGGGACACGAGCACTTCGACCGTCTACATGGAGCTGTCTAGCCTACGGAGT |
| | GAAGATACCGCAGTGTATTACTGCGCAAGGGCGAACTACTATGGGGATTATGTAAACTACTATTAT |
| | GGGATGGACGTCTGGGGACAAGGGACTACCGTGACAGTGTCCTCTGGTGGTGGCGGAAGCGGAGGA |
| | GGAGGGTCAGGTGGAGGCGGGTCAGACATTCAGATGACACAGTCTCCAAGCTCCCTGTCCGCTTCA |
| | GTTGGAGATCGCGTTACAATTACCTGTCAGGCCTCCCAGGACATCAGTAATTACCTGAATTGGTAC |
| | CAGCAGAAACCCGGCAAAGCTCCCAAACTTCTCATATACAAGGCCTCCAGCCTCGAGAGCGGCGTG |
| | CCTAGTAGATTTTCTGGTTCTGGGAGTGGCACAGATTTCACCTTGACCATTTCAAGTTTGCAGCCT |
| | GAGGACTTCGCCACTTATTACTGTCAACAGACTTATACGATACCCATCACATTTGGGCAGGGAACT |
| | CGTTTAGAGATCAAG (SEQ ID NO: 774) |
| TMPRSS4 | GAAGTGCAGCTGCTCGAAAGCGGCGGCGGGCTGGTGCAGCCTGGAGGTTCATTACGGTTGTCATGC |
| Ab36 scFv | GCAGCGAGCGGCTTCACCTTCTCCTCCCGCGCTATGAGTTGGGTGCGACAGGCACCTGGGAAAGGC |
| (VH-VL) | CTGGAGTGGGTATCCAGGATTAATTATGATGGGTCTGCCACAACTTATGCCGACTCTGTTAAGGGC |
| | CGATTTACCATATCCCGCGACAATTCCAAAAACACGCTGTACTTGCAGATGAACTCGCTTAGAGCT |
| | GAGGACACCGCGGTGTACTATTGCGCCAGAGGCATAACTATTTTTGGGGTCTTCGATTACTGGGGA |
| | CAAGGGACACTTGTAACCGTGTCCTCCGGAGGAGGTGGATCAGGAGGCGGCGGTTCTGGCGGAGGA |
| | GGGAGTGACATTCAGATGACTCAGAGCCCATCTTCGCTGTCAGCTAGTGTTGGGGATAGAGTCACC |
| | ATCACCTGTAGGGCTTCTCAGTCAATCTCTACATGGCTCGCCTGGTATCAACAGAAGCCAGGCAAG |
| | GCACCCAAACTGCTGATCTACCGGGCCAGCAACCTCCAGAGCGGCGTGCCGAGTCGTTTTAGCGGC |
| | TCTGGAAGTGGTACTGATTTCACGCTCACCATCTCCAGCCTACAGCCCGAGGACTTTGCCACATAT |
| | TACTGTCAACAAAGCTACAGCACACCCTTAACTTTCGGCGGAGGCACAAAGGTCGAAATCAAG |
| | (SEQ ID NO: 775) |
| TMPRSS4 | GAAGTCCAACTGCTCGAATCTGGCGGCGGCTTGGTTCAGCCTGGCGGGAGCTTGCGGCTCTCATGT |
| Ab37 scFv | GCTGCCAGCGGGTTCACCTTCTCCTCCTATGCCATGCATTGGGTTCGCCAGGCACCCGGCAAGGGC |
| (VH-VL) | TTAGAGTGGGTGTCTTACATAAGTAGTAGTGGCAGCACCGTGTACTACGCCGATTCTGTTAAAGGG |
| | AGGTTCACCATCTCCCGGGATAATAGCAAGAATACGCTGTACCTCCAGATGAACTCCCTGAGAGCA |
| | GAGGACACCGCCGTGTACTATTGCGCCAGAGTATCTAACGTCACTCCCAGGAGCGGGTTTGGCTAT |
| | TGGGGTCAGGGAACGCTTGTAACAGTGTCCAGTGGAGGTGGAGGAAGCGGCGGAGGTGGATCGGGA |
| | GGCGGAGGTTCAGACATTCAGATGACTCAGAGCCCGAGTTCACTGTCAGCTTCGGTCGGAGATCGC |
| | GTGACCATAACTTGCAGGGCTTCTCAGTCAATCAGCCGATACCTGAACTGGTATCAACAGAAGCCT |
| | GGGAAAGCGCCTAAGCTGCTGATCTACTCAGCATCCACTCTCCAAAGCGGCGTGCCCTCCCGTTTT |
| | AGCGGCTCTGGGTCTGGGACAGACTTCACACTAACAATAAGTAGTTTACAGCCAGAGGACTTTGCT |
| | ACCTATTATTGTCAACAGGCCCACTCTTTCCCGCCATCCTTTGGGCAGGGTACAAAACTTGAAATT |
| | AAG (SEQ ID NO: 776) |
| TMPRSS4 | CAAGTGCAGCTGGCTCAGTCTGGGGCAGAAGTCAAAAAACCTGGCGCCTCTGTAAAAGTGTCCTGC |
| Ab38 scFv | AAGGCCTCCGGCTATACCTTCACCCGGCACTACATCCAGTGGGTGCGGCAGGCACCTGGACAAGGC |
| (VH-VL) | TTAGAGTGGATGGGGTGGATTAATCCCAACTCCGGCAATACGGGCTATGCCCAAAAATTCCAAGGG |
| | CGCGTTACCATGACTCGAGATACGTCCACATCTACGGTCTACATGGAACTGAGTTCCTTGCGCTCG |
| | GAGGACACAGCGGTGTATTACTGCGCTAGAGGCAGGCAATGGCTCCGTGGGGAATACTTCCAGCAC |
| | TGGGGACAAGGGACCCTTGTCACAGTTTCCAGCGGAGGTGGCGGGTCAGGTGGCGGTGGATCAGGC |
| | GGAGGAGGAAGTGACATCCAGATGACCCAGTCACCATCTTCCCTGTCAGCGAGCGTGGGCGACAGA |
| | GTAACTATTACCTGTCAGGCTAGTCAAGACATTAGCAGATACCTCAACTGGTATCAGCAAAAGCCC |
| | GGGAAGGCACCCAAGCTGCTCATCTACGGTGCCAGCAACCTGCTGAGCGGCGTGCCTAGCAGGTTT |
| | TCTGGAAGTGGTTCTGGGACAGATTTCACTCTTACAATAAGTAGCCTACAGCCGGAGGATTTTGCC |
| | ACCTACTATTGTCAACAAACTCATACTACTCCATATACCTTTGGGCAAGGCACAAGGTTAGAGATC |
| | AAG (SEQ ID NO: 777) |
| TMPRSS4 | CAAGTGCAGCTGGTACAGAGCGGCGCAGAGGTCAAAAAGCCTGGCGCGAGCGTCAAAGTGTCCTGC |
| Ab39 scFv | AAGGCCTCCGGCGGGAGTTTTAGCGGCTATGCTGTGTCCTGGGTTCGGCAGGCGCCTGGGCAGGGT |
| (VH-VL) | TTGGAGTGGCTGGGCGTAATTAATCCATCCGATTCTTGGACCGCCTTCGCTCAGAAATTTCAGGGC |
| | CGCGTTACTATGACTCGAGATACTTCTACTTCTACCGTCTACATGGAACTTAGTAGTCTCAGGTCT |
| | GAGGACACGGCAGTGTACTATTGCGCTAGGGAACGCGAGGATGATGCCTTCGACATCTGGGGACAA |
| | GGGACCACGGTCACAGTGTCCTCCGGCGGTGGAGGAAGCGGAGGAGGAGGTTCAGGAGGCGGAGGG |
| | TCAGACATACAGATGACACAGTCGCCCTCATCACTGTCAGCCTCTGTTGGGGATAGAGTGACCATT |
| | ACCTGTAGAGCTTCGCAAGGGATTAGGAACTGGTTGGCCTGGTACCAGCAGAAGCCCGGCAAAGCA |
| | CCAAAGCTGCTGATCTACAGAGCAAGCACGCTACAGAGCGGCGTGCCTAGCCGTTTTAGCGGGAGC |
| | GGTAGTGGCACAGACTTCACCCTTACCATCTCCTCCTTACAACCCGAAGACTTCGCCACATACTAT |
| | TGTCAACAGAGTTATACAACTCCGTTTACATTCGGGCAGGGTACAAAGCTCGAGATCAAG |
| | (SEQ ID NO: 778) |
| TMPRSS4 | CAAGTGCAGCTCGTTCAGTCTGGGGCAGAAGTGAAAAAACCTGGGGCGAGTGTCAAAGTGTCCTGC |
| Ab40 scFv | AAGGCCTCAGGCGGCACATTCTCAAGTTATGCCATCTCCTGGGTTCGGCAGGCTCCAGGCCAGGGC |
| (VH-VL) | TTGGAATGGATGGGGATAATTAATCCGCGTGGCGGGTCAACCAACTACGCTCAGAAGTTCCAAGGG |
| | CGGGTAACAATGACCAGAGATACCAGCACTAGCACCGTCTACATGGAGCTCAGCTCCCTACGCTCA |
| | GAAGATACCGCCGTGTACTATTGTGCAAGGGAAGGTTCTAGCTGGTATTATGACGCCTTTGATATA |
| | TGGGGACAAGGAACTATGGTCACAGTGTCCTCCGGCGGTGGAGGATCTGGTGGAGGCGGAAGTGGA |
| | GGTGGAGGGAGTGACATTCAGATGACTCAGAGCCCTAGCTCGCTGTCGGCTTCTGTAGGCGATAGA |
| | GTGACTATTACTTGCCGAGCTTCTCAGAGTATTTCCAGCTACCTGAATTGGTACCAGCAGAAGCCC |
| | GGCAAAGCACCCAAGCTGCTGATCTACGCGGCCTATAACCTTCAATCTGGGGTGCCATCAAGGTTC |
| | AGCGGCTCCGGGTCTGGCACAGACTTCACCCTTACAATCAGTAGCTTACAGCCCGAGGACTTTGCC |
| | ACCTACTATTGTCAACAGTCCTACTCAATCCCTTTTACGTTTGGAGGTGGCACGAAGGTTGAGATC |
| | AAG (SEQ ID NO: 779) |
| TMPRSS4 | CAGGTCCAGCTCGTACAGAGTGGCGCAGAGGTCAAAAAACCGGGCGCCAGCGTTAAAGTGTCCTGC |
| Ab41 scFv | AAAGCCTCCGGTGGCACATTCTCCTCCTACGCTATCTCCTGGGTTCGGCAGGCTCCAGGTCAAGGG |
| (VH-VL) | CTGGAATGGATGGGGTGGATGAATCCCAACTCCGGCGATACACATTATGCCCAAAAGTTTCAGGGC |
| | CGCGTAACCATGACTAGGGACACCTCTACGTCAACTGTCTACATGGAGCTGTCATCACTAAGATCT |
| | GAAGATACTGCCGTGTACTATTGTGCCCGAGAAGGGTCTAGTTGGTACTACGATGCTTTTGATATT |
| | TGGGGACAAGGCACCCTTGTGACTGTGTCGTCCGGAGGTGGAGGCTCCGGAGGAGGAGGGTCAGGT |
| | GGAGGAGGCTCTGACATTCAGATGACACAGAGCCCTAGCAGTCTGTCAGCCAGCGTTGGCGACAGA |
| | GTGACCATCACCTGTAGGGCTTCTCAGTCAATAAGCCGGTGGTTGGCATGGTATCAACAGAAGCCC |
| | GGGAAGGCACCCAAGCTGCTGATCTACGCAGCGAGCACCCTCCAGACCGGCGTGCCATCGCGTTTT |
| | AGCGGGTCTGGCAGCGGCACAGACTTCACCCTTACAATTAGTAGTTTACAGCCTGAGGACTTTGCG |
| | ACTTACTATTGCTTGCAGCACAGCTCTTATCCTTTCACGTTCGGGCAAGGGACAAAGGTGGAGATC |
| | AAG (SEQ ID NO: 780) |
| TMPRSS4 | CAGGTCCAGCTCGTCCAGTCTGGGGCTGAAGTGAAAAAACCCGGCGCCAGCGTTAAAGTGTCCTGC |
| Ab42 scFv | AAGGCCTCAGGCTATTCCTTCACCTCCCACTACATGCATTGGGTTCGGCAGGCTCCTGGCCAAGGG |
| (VH-VL) | TTAGAATGGATGGGTTGGATGAACCCGAACTCCGGCAATACCGGTTATGCTCAGAAGTTCCAAGGG |
| | CGAGTCACAATGACTCGTGATACTAGCACTTCTACAGTGTATATGGAGCTGAGTAGTCTGCGCAGT |
| | GAAGATACTGCCGTATATTACTGTGCACGGCTTGGGCAGCAGCTGGATTACTGGGGACAAGGGACT |
| | CTTGTGACCGTGTCCTCCGGCGGCGGAGGTTCAGGAGGCGGAGGATCAGGAGGAGGAGGCTCTGAC |
| | ATTCAGATGACCCAGTCGCCCTCGTCACTGTCAGCGAGTGTGGGCGATAGAGTAACCATCACCTGT |
| | AGGGCATCTCAGAGCATCCGCAATTACCTCAACTGGTACCAGCAGAAGCCAGGGAAAGCACCCAAG |
| | TTGCTGATTTACGAGGCCTCCAGACTACAGTCTGGTGTGCCGAGCAGGTTTAGCGGTTCCGGGTCT |
| | GGCACAGACTTCACGCTCACAATAAGTAGCTTGCAGCCAGAGGACTTTGCCACCTATTACTGCCAA |
| | CAAAGCTATAGCGCTCCACCTACATTTGGGCCTGGCACGAAGGTTGACATCAAG |
| | (SEQ ID NO: 781) |
| TMPRSS4 | CAAGTGCAGCTCGTCCAGTCTGGGGCTGAAGTGAAAAAACCGGGTGCCTCCGTCAAGGTGTCCTGC |
| Ab43 scFv | AAGGCCTCAGGCTACACTTTCATAGGGTACTACATGCATTGGGTACGGCAGGCACCTGGGCAAGGT |
| (VH-VL) | CTAGAATGGATGGGGCGGATTAATCCCAACTCCGGCGAAACTAACTATGCTCAGAAGTTTCAGGGC |
| | CGAGTAACCATGACTAGGGACACCAGCACCAGCACGGTCTACATGGAGCTGTCATCTTTGAGGAGC |
| | GAGGACACCGCTGTGTACTATTGCGCTAGAGTTAGAGTGCGCGGCGTGATACACCCTGGCTTTGAT |
| | CCCTGGGGTCAGGGAACACTTGTCACCGTGTCCTCCGGAGGCGGAGGATCTGGCGGAGGAGGCTCT |
| | GGTGGTGGTGGCAGTGACATTCAGATGACACAGAGTCCCAGCTCCCTGTCAGCCTCGGTTGGGGAC |
| | CGCGTCACCATCACCTGTCAGGCCTCACAAGATATTAGTAATTACCTGAACTGGTATCAGCAAAAA |
| | CCTGGCAAGGCACCAAAGCTGCTGATCTACGCGGCAAGCTCCTTACAGTCTGGGGTGCCCTCGCGT |
| | TTTAGCGGAAGTGGAAGTGGCACAGATTTCACGCTTACTATCTCAAGCCTCCAGCCTGAGGATTTC |
| | GCCACTTATTATTGTCAACAGTCTTATAGCACACCAGTGACATTCGGGCCAGGGACAAAAGTTGAC |
| | ATCAAG (SEQ ID NO: 782) |
| TMPRSS4 | CAAGTGCAGTTAGTTCAGTCTGGTGCGGAGGTGAAAAAACCTGGGGCATCTGTGAAAGTGTCGTGC |
| Ab44 scFv | AAGGCCAGCGGCTATACCTTTCGGAACTACTACATCCACTGGGTCCGCCAGGCTCCTGGTCAGGGC |
| (VH-VL) | CTAGAATGGATGGGCAGGATTAATCCAAACTCAGGCGGCACAAACTACGCCCAGAAGTTTCAGGGA |
| | CGTGTGACTATGACTAGGGATACCTCCACCTCCACCGTCTACATGGAACTGAGTAGTCTCAGATCT |
| | GAGGATACTGCTGTGTACTATTGCGCCCGCGCCAGAATTGCTGTCGCCGTTTCCGGGTTCGGCTAT |
| | TGGGGTCAGGGAACATTGGTAACAGTGTCAAGTGGTGGCGGCGGAAGTGGCGGAGGAGGATCTGGT |
| | GGCGGTGGATCTGACATTCAGATGACTCAAAGCCCATCATCCCTGAGTGCCTCTGTGGGCGACCGG |
| | GTCACAATAACCTGTCAGGCATCACAGGACATCAGCAATTACCTGAATTGGTATCAGCAAAAGCCC |
| | GGGAAAGCTCCCAAGCTGCTGATATACGCAGCGAGCAGCCTCCATAGCGGAGTACCTTCTCGATTC |
| | AGCGGGTCCGGGTCCGGGACCGATTTCACTCTTACAATCAGCTCATTGCAGCCCGAAGATTTTGCA |
| | ACCTATTATTGTCAAGAGTCCTCGTCCTTTCCGTACACGTTCGGGCCAGGGACGAAAGTTGACATC |
| | AAG (SEQ ID NO: 783) |
| TMPRSS4 | CAGGTCCAGCTCGTCCAGAGTGGCGCGGAAGTGAAAAAGCCTGGCGCCAGCGTAAAAGTGTCCTGC |
| Ab45 scFv | AAGGCCTCCGGCTATACCTTTTCACGGTGGTATATGCATTGGGTCCGGCAGGCACCAGGCCAAGGG |
| (VH-VL) | CTGGAATGGATGGGTCGCATTAATCCCAACTCCGGCGGCACTAACTATGCTCAGAAATTCCAAGGG |
| | CGAGTAACTATGACTAGGGACACATCTACCAGCACAGTTTACATGGAGCTGTCAAGTTTGAGGTCT |
| | GAGGATACCGCCGTGTACTATTGCGCCAGAGTTGGCGGTTATGGGTGGTTTGATCCTTGGGGACAA |
| | GGGACCCTGGTGACAGTGTCCTCCGGCGGAGGAGGAAGTGGCGGAGGAGGATCTGGCGGTGGTGGT |
| | AGCGACATTCAGATGACGCAGAGCCCATCTTCCCTATCCGCTTCGGTTGGGGATAGAGTGACTATT |
| | ACCTGTCAGGCAACGCAGGACATACGCAATTACCTGAACTGGTACCAGCAGAAACCCGGCAAGGCT |
| | CCGAAGCTGCTTATCTACGCAACATCATCATTACAAAGCGGAGTGCCATCTCGTTTTTCTGGGAGT |
| | GGGTCAGGCACAGATTTCACCTTGACCATCAGCTCGCTTCAGCCCGAGGACTTCGCCACCTACTAT |
| | TGTCAACAGAGTTACAGCCCTCCGTACACGTTCGGGCAGGGAACAAAGCTCGAAATCAAG |
| | (SEQ ID NO: 784) |
| TMPRSS4 | CAAGTGCAGCTGGTACAGTCAGGCGCCGAAGTCAAAAAGCCCGGAGCCTCGGTTAAAGTGTCCTGC |
| Ab46 scFv | AAGGCCTCTGGTTACACTTTTTCCCGGTATTTCATGCATTGGGTGCGGCAGGCACCTGGCCAGGGT |
| (VH-VL) | TTGGAGTGGATGGGGTGGATTAATCCCAACTCCGGTGGCACCAACTACGCCCAAAAGTTTCAGGGA |
| | AGAGTGACTATGACCAGGGATACCTCAACTTCCACCGTCTACATGGAACTCAGCTCTCTGCGCAGC |
| | GAGGACACCGCTGTGTACTATTGCGCTAGAGTCCGAATTGGGTGGCTCCAGTCACCTCCACTGTAC |
| | TGGGGACAAGGGACACTTGTTACAGTTTCCTCTGGAGGCGGAGGGTCAGGTGGCGGAGGAAGTGGC |
| | GGTGGCGGATCTGACATTCAGATGACACAGTCTCCAAGCAGTTTGAGCGCCAGTGTGGGCGACCGT |
| | GTAACTATCACGTGTAGAGCAAGTCAGTCAATCAGCACATGGCTCGCATGGTATCAACAGAAGCCC |
| | GGCAAAGCTCCAAAACTGCTGATCTACGCGGCGAGCAGTTTACAGTCCGGCGTGCCTTCGAGGTTT |
| | TCTGGGTCCGGGTCCGGGACCGATTTCACCCTTACAATAAGCAGCCTACAGCCCGAGGATTTTGCT |
| | ACGTACTATTGTCAACAGTCTTATGGGTTCCCGTGGACTTTCGGCCAGGGTACAAAGGTCGAAATC |
| | AAG (SEQ ID NO: 785) |
| TMPRSS4 | CAGGTCCAGCTGGTGCAGAGCGGTGCAGAAGTCAAGAAGCCCGGTGCAAGCGTAAAAGTGTCCTGC |
| Ab47 scFv | AAGGCCTCCGGGTACACCTTCACCGGTTATTTCATGCATTGGGTCCGGCAGGCTCCTGGCCAAGGG |
| (VH-VL) | TTGGAATGGATGGGCTGGATGAATCCCAACTCCGGGAATACCGGCTACGCTCAAAAATTTCAAGGG |
| | CGCGTGACCATGACAAGGGATACAAGCACCTCCACCGTGTATATGGAGCTCAGCTCTCTGAGATCA |
| | GAGGATACAGCCGTTTATTACTGCGTTCGAGGGAGGACTTGGATTCAGTCAAGCCTGGGGTACTGG |
| | GGTCAGGGAACGCTTGTGACAGTCTCATCTGGAGGAGGCGGAAGCGGAGGAGGCGGAAGTGGAGGT |
| | GGCGGAAGCGACATTCAGATGACTCAGTCTCCAAGCTCGCTGTCAGCTAGTGTTGGCGACAGAGTA |
| | ACTATCACTTGTCGGGCCTCGCAGTCCATCTCCTCATATCTCAACTGGTATCAACAGAAACCGGGC |
| | AAGGCACCTAAGCTGCTCATTTACGCGGCCTCCTCCCTACAGTCTGGGGTGCCTAGTCGTTTTAGC |
| | GGGTCTGGCTCTGGGACCGACTTCACTCTTACAATAAGTAGTTTGCAGCCAGAAGATTTCGCCACT |
| | TACTATTGTCAACAGAGTTACTCTACGCCCTTAACCTTTGGCGGCGGCACAAAAGTGGAGATCAAG |
| | (SEQ ID NO: 786) |
| TMPRSS4 | CAAGTGCAGCTGGTCCAGTCTGGGGCGGAAGTGAAAAAGCCCGGAGCTAGTGTAAAGGTGTCCTGT |
| Ab48 scFv | AAAGCCAGCGGCTACACCTTCACCGGTTATTACCTGCATTGGGTCCGGCAGGCTCCTGGCCAGGGC |
| (VH-VL) | CTGGAGTGGATGGGCTGGATTTCCGCATATAACGGAAACACAAATTACGCCCAGAACCTGCAAGGC |
| | CGCGTGACCATGACCAGGGACACAAGCACTAGCACTGTCTACATGGAGTTGTCTAGCTTGAGAAGC |
| | GAAGATACCGCTGTGTACTATTGCGCCCGACACTCTTACTCGGGCTCATACTCAACGCTACCCTAT |
| | TATGGGATGGATGTTTGGGGTCAAGGGACAACGGTCACAGTATCCTCTGGAGGCGGTGGCAGCGGA |
| | GGAGGCGGGTCTGGAGGTGGTGGATCAGACATTCAGATGACCCAGTCACCAAGTTCCTTATCCGCA |
| | AGCGTTGGGGATCGTGTTACAATTACTTGCAGGGCCTCGCAAGGGATCTCTAATTATCTCGCTTGG |
| | TACCAGCAGAAACCTGGGAAAGCACCCAAGCTGCTGATCTACACTGCAAGCACACTTTTTCCAGGA |
| | GTGCCGTCAAGATTCTCTGGGTCCGGGAGTGGCACTGACTTCACCCTTACCATCTCCTCCCTCCAG |
| | CCTGAGGACTTTGCCACATATTATTGTCAACAGAGTTACTCCATACCACTCACGTTTGGCGGCGGA |
| | ACAAAaGTtGAAATCAAG (SEQ ID NO: 787) |
| TMPRSS4 | CAGGTCCAGTTGGTACAGAGCGGCGCCGAAGTGAAAAAGCCTGGGGCGTCCGTCAAAGTGTCTTGC |
| Ab49 scFv | AAGGCCTCCGGCTATACATTCACCGGGTACTACATGCATTGGGTGCGGCAGGCACCTGGCCAGGGT |
| (VH-VL) | CTAGAATGGATGGGCCGGATCAATCCCAACTCCGGCGGCACAAACTATGCTCAGAAATTTCAAGGT |
| | CGCGTCACCATGACCCGTGACACAAGTACGAGCACCGTCTACATGGAGCTGTCCTCCCTCAGGAGC |
| | GAGGATACAGCCGTGTACTATTGTGCAAGGGAGCGCGCCGGCTATAGCAGCGGGCAGTTCGATTAT |
| | TGGGGACAAGGGACTCTGGTAACTGTGTCCTCCGGAGGCGGAGGATCAGGCGGAGGAGGCTCAGGA |
| | GGTGGAGGTTCTGACATTCAGATGACTCAATCTCCCTCGTCACTGTCAGCTAGTGTTGGGGATAGA |
| | GTGACTATTACCTGCCGAGCCAGTCAGTCAATATCTAACTGGCTCGCATGGTACCAGCAGAAGCCA |
| | GGGAAGGCTCCCAAACTGCTGATCTACGCCGCGAGCACCCTTCAGAATGGCGTGCCGTCTAGATTT |
| | AGCGGTTCTGGGTCTGGGACCGACTTTACACTTACTATCAGTAGTTTACAACCAGAGGACTTTGCT |
| | ACTTATTACTGTCAACAGAGCTACACCTTCCCTATTACGTTCGGCCAGGGAACAAAAGTTGAAATC |
| | AAG (SEQ ID NO: 788) |
| TMPRSS4 | CAGGTCCAGCTGGTTCAGTCTGGCGCGGAAGTTAAAAAGCCAGGCGCCTCCGTCAAAGTGTCCTGC |
| Ab50 scFv | AAGGCCTCCGGCTATACTTTCACCGGTTATTACATGCATTGGGTGCGGCAGGCACCTGGGCAAGGG |
| (VH-VL) | CTGGAATGGATGGGATGGATTAACCCGAACTCCGGAGGCACACACTATGCCCAAAAGTTTCAGGGA |
| | CGGGTTACAATGACTCGTGACACTTCAACTAGCACCGTCTACATGGAGCTTAGTAGTTTGAGGTCA |
| | GAGGACACCGCTGTGTATTACTGCGCTAGAGTGCGAATCGGGTGGCTGCAGAGTCCACCACTGTAC |
| | TGGGGACAAGGGACTTTGGTAACAGTGTCAAGCGGCGGAGGTGGATCAGGAGGCGGCGGTAGCGGA |
| | GGAGGTGGATCTGACATTCAGATGACCCAGTCGCCGTCCTCCCTATCCGCCAGTGTCGGTGATCGC |
| | GTAACCATTACGTGTAGGGCCTCTCAAGGGATCAGCAATTACCTCGCATGGTACCAGCAGAAACCC |
| | GGGAAGGCACCCAAACTGCTGATCTACGCTACAAGCAGACTCCAGTCAGGCGTGCCCTCTCGCTTC |
| | TCTGGCAGCGGGTCTGGCACCGATTTCACCCTTACAATAAGTAGCCTCCAGCCTGAGGATTTTGCT |
| | ACGTATTATTGTCAACAGAGCTACAAGACTCCCTTAACCTTTGGCGGCGGGACAAAAGTGGAAATC |
| | AAG (SEQ ID NO: 789) |
| TMPRSS4 | CAAGTGCAGCTGGTCCAGAGCGGTGCCGAAGTGAAAAAGCCTGGGGCGTCCGTGAAAGTGTCCTGT |
| Ab51 scFv | AAGGCCAGCGGATATACCTTCACCAACTACTACATGCATTGGGTCCGGCAGGCTCCCGGCCAAGGG |
| (VH-VL) | CTGGAATGGATGGGGTGGATTAATCCAAAATCTGGCGGCACTTCTTATGCACAGAAGTTCCAGGGC |
| | CGCGTTACTATGACTAGGGATACAAGCACCAGCACTGTCTACATGGAACTGTCGAGTTTGAGAAGT |
| | GAGGATACAGCAGTGTATTACTGCGCCAGCGGGAAGCAATGGCTCGTAGGAGGTCGATTCGACTAT |
| | TGGGGTCAGGGAACACTTGTCACCGTTTCATCCGGAGGAGGAGGGTCTGGTGGAGGAGGGTCTGGA |
| | GGTGGCGGGTCAGACATTCAGATGACGCAGAGTCCAAGCTCCTTGTCCGCTTCTGTGGGCGATAGA |
| | GTAACCATTACTTGCAGGGCTTCACAGAGCATCTCTTCATACCTGAACTGGTACCAGCAGAAACCC |
| | GGGAAGGCACCCAAACTTCTCATCTACGCTGCCTCCTCCCTACAATCCGGCGTGCCGAGTCGTTTT |
| | TCAGGCTCGGGCTCTGGCACCGACTTCACACTCACGATAAGTAGTTTACAGCCTGAGGACTTTGCC |
| | ACCTACTATTGTCAACAGAGCTATAGCACACCTCTGACCTTTGGCGGCGGGACAAAGGTTGAGATC |
| | AAG (SEQ ID NO: 790) |
| TMPRSS4 | CAGGTCCAGCTGGTTCAGTCTGGCGCAGAAGTGAAAAAGCCTGGGGCATCTGTAAAGGTGTCCTGC |
| Ab52 scFv | AAGGCCTCCGGATATACCTTCACTAGATATTACATCCACTGGGTGCGGCAGGCACCTGGGCAAGGG |
| (VH-VL) | CTGGAATGGATGGGGTGGATGAATCCGAACTCCGGCAATACCGGGTTTGCCCAAAAACTGCAAGGG |
| | CGAGTAACAATGACCAGGGATACCAGCACAAGCACGGTCTACATGGAGCTCAGCTCCCTCCGCTCT |
| | GAGGACACCGCTGTGTACTATTGTGCCCGCGGTCCCTTTCCTAGAGGACGGCTCGACCTGTGGGGA |
| | CAAGGCACACTTGTCACAGTGTCCTCCGGTGGTGGAGGAAGCGGAGGAGGCGGTTCAGGCGGAGGA |
| | GGCAGTGACATTCAGATGACTCAGTCTCCAAGCTCCTTGTCAGCGAGTGTTGGGGATAGAGTGACA |
| | ATAACTTGCAGGGCTAGTCAGGGTATTAGTCGGTGGCTAGGCTGGTACCAGCAGAAACCCGGGAAG |
| | GCTCCAAAACTGCTGATCTACGGCGCCAGCAACTTGCAGACTGGGGTGCCCTCGCGTTTCTCAGGC |
| | TCAGGCTCTGGGACTGACTTCACCCTTACCATTAGTAGCTTACAGCCCGAAGATTTTGCCACCTAT |
| | TATTGTCAACAGTCATACAGCTCTCCAAGGACGTTCGGCCAGGGTACAAAGGTTGAGATCAAG |
| | (SEQ ID NO: 791) |
| TMPRSS4 | CAGGTCCAGCTGGTCCAGTCAGGCGCCGAAGTGAAAAAGCCTGGGGCGTCCGTGAAAGTGTCCTGC |
| Ab53 scFv | AAAGCCTCCGGCTATACATTTACCCGATACTACATGCATTGGGTGCGGCAGGCTCCAGGCCAAGGG |
| (VH-VL) | CTGGAATGGATGGGGATAATCAATCCCACAGGCGGGTCTACATCGTATGCACAGAAGTTCCAAGGG |
| | CGCGTCACTATGACTCGAGACACCTCTACTAGCACGGTCTACATGGAACTAAGTAGCCTCCGCAGC |
| | GAGGATACCGCCGTGTATTACTGCGCTAGAGGCAGGACCTGGATTCAATCTAGCCTGGGGTATTGG |
| | GGTCAGGGAACACTTGTTACCGTGTCCTCCGGAGGAGGAGGTTCCGGTGGCGGAGGGAGTGGAGGA |
| | GGTGGATCTGACATACAGATGACACAGTCTCCTAGTTCCTTGTCAGCTTCGGTTGGCGATAGAGTA |
| | ACCATTACATGCAGGGCCTCTAGATCAATCAACAGGTGGTTGGCGTGGTATCAACAGAAACCCGGG |
| | AAAGCACCAAAACTGCTGATCTACGGAGCATCAACTTTACAGAGTGGCGTGCCTAGCCGTTTTTCT |
| | GGCAGCGGTAGTGGTACTGACTTCACACTTACGATTAGTAGCCTCCAGCCGGAGGATTTCGCAACA |
| | TACTATTGTCAACAGAGCTACAGCACTCCCACCTTTGGCGGCGGCACGAAAGTTGAGATCAAG |
| | (SEQ ID NO: 792) |
| TMPRSS4 | CAAGTACAGTTAGTGCAGAGCGGAGCCGAAGTTAAAAAACCTGGGGCGTCAGTCAAAGTCTCATGC |
| Ab54 scFv | AAGGCCTCCGGCTATACCTTCACCTCATACTACATGCAGTGGGTTCGGCAAGCTCCCGGGCAGGGC |
| (VH-VL) | CTGGAGTGGATGGGCTGGATGAACCCTAATTCCGGCAATACTGGTTATGCACAGAAGTTCCAGGGC |
| | CGCGTGACTATGACCAGAGATACCTCCACTTCCACCGTCTACATGGAGCTAAGCTCCCTCCGTAGC |
| | GAAGACACTGCTGTGTACTATTGTGCACGAGTGCGCATCGGGTGGCTGCAGAGTCCTCCGTTGTAC |
| | TGGGGTCAAGGGACACTCGTGACAGTGTCCAGCGGTGGAGGTGGATCTGGCGGTGGAGGATCTGGA |
| | GGCGGAGGCTCTGACATTCAGATGACCCAGTCACCTTCTTCACTGTCAGCCTCTGTGGGCGACCGG |
| | GTTACAATTACATGCAGAGCTTCGCAGGGAATCTCCAACTATCTGGCTTGGTATCAGCAAAAGCCC |
| | GGCAAAGCACCCAAGCTCCTTATCTACGCAGCGAGCAGTTTGCAGTCTGGGGTACCCAGTAGGTTT |
| | AGCGGGTCTGGGAGTGGCACAGATTTTACTCTGACGATAAGTAGCCTTCAGCCAGAGGATTTCGCC |
| | ACGTACTATTGTCAACAGTCCTACTCGATTCCATTCACGTTTGGGCCAGGGACAAAAGTCGACATC |
| | AAG (SEQ ID NO: 793) |
| TMPRSS4 | CAAGTGCAGCTCGTTCAGAGCGGCGCGGAGGTCAAGAAGCCTGGCGCCTCAGTCAAAGTCTCTTGC |
| Ab55 scFv | AAGGCCTCCGGCTATACTTTCACCACGTATTACATGCATTGGGTGCGGCAGGCTCCCGGCCAGGGC |
| (VH-VL) | CTGGAATGGATGGGAATTATTAATCCGAGCGGCGGGAGTACAAGCTACGCTCAGAAATTCCAGGGA |
| | CGGGTGACTATGACCCGAGACACCAGCACATCTACTGTCTACATGGAGCTGAGTAGCTTGCGCTCA |
| | GAGGACACCGCCGTGTACTATTGTGCACGCGGGAGGAGCTGGTATAGAAGCAACGTAGACTATTGG |
| | GGACAAGGGACACTTGTAACAGTGTCAAGTGGCGGAGGCGGGTCCGGTGGAGGTGGTTCAGGAGGT |
| | GGAGGTTCTGACATTCAGATGACTCAGAGTCCATCGTCACTGTCAGCCAGCGTTGGGGATAGAGTG |
| | ACCATCACTTGCAGGGCTAGTCAAAGTATCTCCTCCTGGCTCGCATGGTACCAGCAGAAACCTGGG |
| | AAGGCTCCTAAACTGCTGATATACGCAGCGTCCTCCCTTCAGTCTGGAGTGCCCTCGAGATTTAGC |
| | GGCTCTGGCTCCGGCACAGATTTCACCCTAACAATATCCAGCTTGCAGCCCGAAGATTTTGCCACC |
| | TATTACTGTCAACAGTCTTACTCTACACCAAGGACGTTTGGTCAAGGGACCCGTTTAGAAATCAAG |
| | (SEQ ID NO: 794) |
| TMPRSS4 | CAGGTTCAACTGGTCCAGTCTGGGGCAGAAGTGAAGAAGCCTGGAGCTTCTGTTAAAGTGTCCTGC |
| Ab56 scFv | AAGGCCTCCGGTCACACCTTCACTCGATATTACATGCATTGGGTTCGGCAGGCACCTGGCCAGGGT |
| (VH-VL) | TTGGAGTGGATGGGGTGGATTAATCCGAACTCCGGGAATACTGGGGACGCTCAGAAATTTCAGGGC |
| | CGCGTGACCATGACACGGGATACCAGCACCAGCACCGTCTACATGGAACTCAGCTCCCTGCGCTCC |
| | GAAGATACGGCAGTGTACTATTGCGCTAGGGACAGAGGCATAGTGGTGGTGCCCGCTGCCATCGGA |
| | GGCATGGACGTATGGGGACAAGGCACCATGGTCACAGTGTCAAGTGGAGGAGGCGGTTCAGGCGGT |
| | GGCGGGTCTGGTGGTGGAGGATCAGACATTGTCATGACTCAAAGTCCATTGAGTCTGCCAGTGACA |
| | CCTGGGGAGCCCGCGAGCATCTCTTGTAGGAGCAGCCAGTCCCTCCTGCACTCCAACGGCTATAAC |
| | TATCTCGACTGGTACCTACAGAAACCCGGGCAGAGCCCTCAGTTACTGATCTACCTTGGGTCGAAT |
| | AGGGCCTCTGGGGTGCCAGATAGATTCTCAGGATCTGGAAGTGGCACTGATTTCACACTGAAGATA |
| | AGTAGAGTCGAGGCCGAGGATGTTGGCGTCTACTATTGTATGCAGGCCCTTCAGACACCCATTACG |
| | TTTGGCCAAGGCACTCGTCTGGAGATCAAG (SEQ ID NO: 795) |
| TMPRSS4 | CAAGTGCAGCTCGTCCAATCTGGGGCTGAAGTGAAAAAGCCTGGAGCCTCCGTGAAGGTGTCCTGC |
| Ab57 scFv | AAGGCCTCCGGCTACACTTTCACGGGCTATTTCATGCATTGGGTGCGGCAGGCACCCGGGCAGGGA |
| (VH-VL) | CTGGAGTGGATGGGCAGGATTAATCCCAACTCCGGCGGGACAAATTACGCGCAGAAGTTTCAGGGC |
| | CGCGTTACCATGACTAGAGATACCAGCACTTCAACCGTTTACATGGAGCTGAGCAGTCTGCGCAGC |
| | GAGGACACGGCTGTCTACTATTGCGCTAGAGGAAAAGGGCGATATTTCGACCTGTGGGGTAGAGGC |
| | ACACTCGTAACAGTCTCCAGTGGAGGAGGCGGGTCAGGTGGCGGTGGGTCTGGAGGTGGAGGGTCA |
| | GACATTGTTATGACTCAAAGTCCCTTGTCCCTGCCCGTGACTCCTGGCGAACCAGCCTCAATCTCC |
| | TGTCGAAGCTCTCAGAGCCTTCTCCACTCTAATGGCTATAACTATCTGGACTGGTACCTTCAAAAA |
| | CCGGGCCAGAGTCCTCAGCTCCTAATCTACTTGGGATCTAACAGGGCCTCTGGGGTGCCAGATAGG |
| | TTTAGCGGTAGTGGCAGCGGCACAGATTTCACCCTGAAAATTTCGCGGGTAGAAGCAGAGGATGTG |
| | GGTGTCTACTATTGTATGCAGGGAACACATTGGCCAATAACCTTTGGGCAGGGAACCCGTTTAGAG |
| | ATCAAG (SEQ ID NO: 796) |
| TMPRSS4 | CAGGTCCAACTGGTCCAGTCTGGGGCAGAAGTGAAAAAACCCGGCGCCTCCGTAAAAGTGTCCTGC |
| Ab58 scFv | AAGGCCTCCGGCTACACTTTCACCCGATACTATCTCCACTGGGTCCGGCAGGCTCCCGGTCAGGGC |
| (VH-VL) | TTAGAGTGGATGGGGTGGGTTAGTGCATACAATGGAAATACAAACTATGCGCAGAAATTCCAAGGG |
| | CGCGTGACCATGACCCGAGACACCAGCACTTCTACTGTCTATATGGAGCTTTCCTCCCTCAGGAGT |
| | GAGGATACTGCCGTGTACTATTGCGCTAGAGGTTACTGCTCAGGCGGGTCATGTTATTGGTTTGAT |
| | CCCTGGGGTCAAGGGACGCTTGTGACCGTGTCCTCCGGAGGAGGCGGAAGCGGTGGAGGAGGATCA |
| | GGCGGTGGAGGCTCTGACATTGTGATGACCCAGTCTCCGCTGTCACTCCCAGTGACACCTGGCGAA |
| | CCCGCTTCAATAAGTTGTAGAAGCAGTCAGTCTTTGCTGCATAGCAACGGCTACAACTATCTAGAT |
| | TGGTACTTGCAGAAGCCTGGGCAGTCGCCACAACTGCTGATCTACCTGGGCAGCAATAGGGCATCT |
| | GGGGTGCCTGACCGCTTTAGCGGCAGCGGTAGTGGCACAGACTTCACGCTGAAAATTAGCCGTGTA |
| | GAGGCCGAAGATGTTGGGGTCTACTATTGTATGCAGGCCCTCCAGACTCCATTAACATTTGGACAA |
| | GGCACAAAGGTTGAGATCAAG (SEQ ID NO: 797) |
| TMPRSS4 | CAGGTTCAGCTAGTTCAGAGCGGAGCCGAGGTCAAGAAGCCCGGAGCGTCTGTGAAAGTGTCCTGC |
| Ab59 scFv | AAAGCCTCCGGAGGCACATTCTCCTCCTATACTTTGAGTTGGGTTCGGCAGGCACCTGGCCAGGGC |
| (VH-VL) | CTGGAGTGGATGGGATGGATTCACCCGAAAAGCGGCGTGACCAAGAATGCACAGAAATTCCAAGGG |
| | CGGGTGACTATGACCCGAGATACTTCCACGTCTACCGTCTACATGGAACTCAGCTCACTGCGCTCA |
| | GAGGACACCGCTGTGTACTATTGCGCTAGAGGCTGGGTGTACGGCAGGATGGACGCCTGGGGTCAG |
| | GGTACTACGGTCACAGTCAGTAGCGGAGGTGGCGGATCTGGAGGAGGCGGGTCTGGCGGTGGAGGG |
| | TCAGAAATCGTCATGACCCAGTCTCCCGCCACACTGAGTGTATCGCCTGGGGAGCGCGCCACTCTG |
| | AGTTGTAGAGCAAGCCAGAGCGTGTCGTCCAATTACCTGGCTTGGTATCAGCAAAAGCCTGGGCAG |
| | GCACCCAGGTTGCTTATCTACGGCGCGTCAACTAGGGCTACCGGGATACCAGCCCGTTTTTCCGGG |
| | TCTGGTTCAGGGACCGAATTCACGCTCACAATTAGCAGTTTACAGAGCGAAGATTTTGCCGTGTAC |
| | TATTGTCAACAATACGGGACACTTCCATATACCTTTGGCCAAGGCACAAAAGTGGAGATCAAG |
| | (SEQ ID NO: 798) |
| TMPRSS4 | CAGGTCCAGCTCGTGCAGTCCGGAGCAGAAGTTAAGAAGCCTGGCGCGTCTGTGAAAGTTTCCTGC |
| Ab60 scFv | AAGGCCTCAGGTTACTCCTTCACCACCTATTACATCCACTGGGTGCGGCAGGCTCCCGGGCAGGGC |
| (VH-VL) | TTGGAGTGGATGGGTATTATTAACCCATCTGGCGGGTCTACAAGCTACGCTCAAAAGTTTCAGGGT |
| | CGGGTCACTATGACACGAGATACTAGCACATCTACCGTCTACATGGAGCTGAGCAGTCTGCGCAGC |
| | GAGGACACCGCTGTGTACTATTGCGCCCGCGGCGGCTACTATGGCTCCGGATACAATTCAGTCGGT |
| | TATTGGGGACCAGGGACGCTTGTAACAGTATCAAGCGGAGGAGGCGGTAGCGGTGGCGGCGGGAGT |
| | GGAGGAGGTGGAAGTGAAATAGTGATGACCCAGTCACCCGCCACCCTGAGTGTTAGCCCTGGGGAA |
| | AGGGCAACCCTGAGTTGTAGAGCGTCGCAGTCGGTGTCCTCCAACACCCTGGCATGGTATCAGCAA |
| | AAACCCGGGCAGGCTCCTAGGCTCCTAATCTACGGCGCCTCTACTCGTGCCACTGGCATTCCCGCC |
| | AGATTTAGCGGGAGCGGGTCCGGCACAGAGTTTACGCTTACAATAAGTTCCTTGCAGTCAGAAGAT |
| | TTCGCAGTGTACTATTGTCAACAATATGGATCTTCTCCGTTAACGTTCGGGCCAGGCACAAAAGTG |
| | GACATCAAG (SEQ ID NO: 799) |
| TMPRSS4 | CAGGTTCAGTTGGTCCAGTCTGGCGCGGAAGTAAAAAAGCCTGGAGCGTCAGTGAAAGTGTCGTGC |
| Ab61 scFv | AAGGCTAGTGGCTATACCTTCACCAATTACTACATGCATTGGGTGCGGCAGGCACCAGGCCAGGGC |
| (VH-VL) | TTGGAGTGGATGGGCTGGATGAATCCCAACTCCGGGAACACCGGCTATGCTCAGAACCTGCAAGGG |
| | CGAGTCACAATGACCCGAGATACCAGCACTAGCACTGTCTACATGGAGCTGTCCAGCCTGCGCTCA |
| | GAGGACACCGCCGTGTACTATTGTGCCCGCGGGAGGACTTGGTTTAGATCCGGAATGGACGTGTGG |
| | GGTCAGGGAACGACGGTCACAGTGTCCAGTGGAGGTGGTGGCTCAGGCGGAGGAGGGAGTGGAGGC |
| | GGAGGAAGCGAGATTGTTATGACTCAGTCTCCGGCCACACTGAGTGTTTCTCCTGGCGAAAGGGCA |
| | ACTCTCAGTTGCAGAGCATCTCAAAGCGTGTCCTCCTACCTAGCTTGGTATCAACAGAAACCCGGG |
| | CAGGCACCTAGGCTGCTCATCTACGGCGCCTCGACCAGAGCCACCGGGATTCCCGCCCGTTTTAGC |
| | GGGTCTGGGAGCGGGACTGAGTTCACCTTAACAATAAGTAGCCTTCAGTCAGAAGACTTCGCTGTG |
| | TACTATTGTCAACAGTATGATATATCTGTGACGTTTGGGCCAGGCACAAAGGTCGATATCAAG |
| | (SEQ ID NO: 800) |
| TMPRSS4 | CAGGTCCAGCTGGTTCAGTCTGGCGCGGAAGTGAAAAAGCCGGGCGCCTCCGTCAAAGTGTCCTGC |
| Ab62 scFv | AAGGCCTCCGGTTACACCTTCACTGACTATTACATCCACTGGGTCCGGCAAGCACCCGGGCAGGGC |
| (VH-VL) | CTGGAATGGATGGGGTGGATTTCTACTTATAATGGGAACACGAATTATGCACAGAAACTCCAGGGC |
| | CGCGTCACTATGACCCGGGATACAAGCACTAGCACCGTCTACATGGAGCTAAGTAGCTTGCGTAGC |
| | GAGGACACCGCTGTGTATTACTGTGCCAGAGGCATGGTGCGAGGCATGGATGTATGGGGACAAGGA |
| | ACAATGGTTACTGTTTCTAGTGGAGGCGGCGGTTCAGGTGGAGGAGGGTCTGGAGGTGGCGGTTCG |
| | GACATTGTGATGACCCAGTCACCAGATAGCCTTGCAGTGTCCCTGGGAGAAAGGGCTACCATTAAT |
| | TGCAAGAGCAGTCAGTCAGTGCTCTACTCAAGCAACAACAAAAACTATTTGGCTTGGTACCAACAG |
| | AAGCCCGGGCAGCCTCCTAAGCTGCTGATCTACTGGGCCTCTACAAGGGAGTCTGGGGTGCCAGAT |
| | CGCTTTTCCGGGTCCGGCTCCGGGACAGACTTCACGCTTACAATAAGTAGTCTCCAGGCTGAGGAC |
| | GTAGCCGTGTACTATTGTCAACAATACTATACTACACCCTGGACCTTTGGCCAGGGAACCAGATTA |
| | GAGATCAAG (SEQ ID NO: 801) |
| TMPRSS4 | CAAGTGCAGCTGGTGCAGTCTGGAGCGGAAGTGAAAAAACCTGGCGCCAGTGTGAAAGTGTCCTGC |
| Ab63 scFv | AAGGCCTCCGGTTATACTTTCACGGGCTATCGGATGCATTGGGTTCGGCAGGCACCTGGGCAAGGA |
| (VH-VL) | CTAGAGTGGATGGGCGTAATTAATCCAAATACTGGGACCGCTCGCTTTGCTCAGAAGTTTCAGGGA |
| | CGAGTCACAATGACTAGGGATACATCAACCAGCACTGTCTACATGGAGCTGAGCAGCCTCAGGAGC |
| | GAAGACACCGCGGTCTACTATTGTGCTTCTGTGGGCGTCTACTGGTATTTTGACCTGTGGGGTAGA |
| | GGCACACTTGTAACCGTGTCCTCCGGAGGAGGCGGGAGTGGAGGTGGAGGTTCCGGTGGTGGAGGG |
| | AGTGACATTGTTATGACACAGTCTCCCGATAGCCTCGCCGTTAGTCTCGGCGAGCGTGCCACGATA |
| | AACTGCAAATCTTCTCAGTCCGTGTTGTACTCAAGCAACAACAAGAATTATCTGGCATGGTACCAG |
| | CAGAAACCTGGGCAACCACCCAAACTGCTGATCTACTGGGCCTCAACCCGCGAATCTGGGGTGCCG |
| | GATAGATTCAGCGGGAGCGGCTCCGGGACAGATTTCACCCTTACTATCTCGAGTTTGCAGGCTGAG |
| | GACGTTGCCGTCTACTATTGTCAACAGTACTATTCAGCACCCTTAACCTTCGGCGGCGGCACAAAA |
| | GTGGAAATCAAG (SEQ ID NO: 802) |
| TMPRSS4 | CAGGTTCAGCTGGTACAGTCAGGCGCCGAAGTGAAAAAACCGGGTGCCAGCGTCAAGGTGTCCTGC |
| Ab64 scFv | AAGGCCTCCGGCTACACCTTCACCGGATATTACATGCATTGGGTCCGGCAGGCACCCGGCCAAGGG |
| (VH-VL) | CTGGAATGGATGGGGATGATTAATCCTAGTGGTGGCGGGACCACATACGCTCAGAAGTTCCAAGGG |
| | CGGGTTACGATGACTCGAGACACCAGCACGTCTACCGTCTACATGGAACTGTCCAGCCTACGCTCT |
| | GAGGATACTGCCGTGTACTATTGTGCAAGGGACAGGAGATCAATGATTACCTTTCGCACAGATTAT |
| | TGGGGTCAGGGAACTCTCGTTACCGTGTCCTCCGGTGGCGGAGGAAGTGGAGGCGGAGGATCAGGA |
| | GGTGGCGGGTCAGACATTGTAATGACTCAGAGCCCAGATAGTCTTGCAGTGTCCTTGGGCGAGAGG |
| | GCTACAATCAACTGCAAGAGCAGTCAGAGCGTGCTCTACTCGTCTAACAACAAAAATTACCTTGCG |
| | TGGTATCAACAGAAACCCGGGCAGCCTCCCAAACTGCTGATCTACTGGGCTTCTACTAGAGAGTCT |
| | GGCGTGCCTGATCGTTTTAGCGGTTCCGGGTCTGGGACAGACTTCACGTTAACAATAAGTAGCCTC |
| | CAGGCTGAGGACGTCGCCGTGTACTATTGTCAACAGTACTATTCAACACCATATACTTTTGGCCAA |
| | GGCACCAAGTTGGAGATCAAG (SEQ ID NO: 803) |
| TMPRSS4 | CAGGTCCAACTGGTCCAGTCTGGCGCGGAGGTTAAAAAGCCTGGGGCTTCAGTCAAAGTGTCGTGT |
| Ab65 scFv | AAGGCCTCGGGCTATACATTCACCGGCTATTACATGCATTGGGTCCGTCAGGCACCTGGGCAAGGG |
| (VH-VL) | CTGGAATGGATGGGTTGGATGAATCCGAACTCCGGGAATACCGGTTATGCCCAAAAATTCCAAGGG |
| | AGAGTAACTATGACACGAGACACCAGCACTAGCACCGTGTATATGGAACTCAGCTCTCTGAGGAGC |
| | GAGGACACAGCCGTGTATTACTGCGCGGGCCGGAAATGGCTGGGCTTGGATTTCTACAACTGGTTT |
| | GATCCTTGGGGTCAGGGAACTCTTGTAACGGTGTCCAGTGGCGGCGGTGGCTCAGGAGGAGGAGGA |
| | TCTGGAGGAGGCGGAAGCGACATTGTGATGACTCAGAGTCCGGATAGCCTTGCAGTGTCTTTGGGC |
| | GAGCGCGCCACTATTAATTGCAAATCCTCCCAGTCCGTTCTCTACAGCTCAAATAACAAGAACTAT |
| | CTCGCATGGTACCAGCAGAAGCCAGGACAGCCACCAAAACTGCTGATATACTGGGCTAGTACCAGA |
| | CAGTCTGGCGTGCCCGACAGGTTTTCAGGGTCCGGGAGCGGCACAGACTTCACGCTAACGATAAGT |
| | AGTTTACAGGCCGAGGATGTCGCTGTGTATTACTGCCAGCAGTACTACTCTACACCCTGGACCTTT |
| | GGGCAAGGCACAAAGGTTGAAATCAAG (SEQ ID NO: 804) |
| TMPRSS4 | CAGGTTCAACTGGTACAGAGCGGCGCGGAAGTGAAAAAACCCGGCTCGTCAGTTAAAGTGTCCTGT |
| Ab66 scFv | AAGGCCTCCGGCGGCACATTCTCCTCCTACGCTATCTCCTGGGTCCGGCAAGCACCCGGGCAAGGT |
| (VH-VL) | CTGGAGTGGGTGGGTGGCATAATGCCCATATTCGGGACAGCGAATTACGCTCAGAAGTTTCAGGGA |
| | AGAGTGACTATTACCGCCGATGAATCTCCATCTACGGCTTATATGGAACTCTCCAGTCTGCGCAGC |
| | GAGGACACCGCTGTGTACTATTGCGCCACCGGGCGTCGAGAGTTGCTGAACTGGGGTCAGGGAACA |
| | CTTGTTACCGTGTCCAGCGGCGGAGGAGGAAGTGGCGGAGGAGGCTCAGGTGGTGGAGGTTCTGAG |
| | ATTGTGATGACTCAGAGCCCTGCTACACTGAGTGTATCTCCTGGGGAACGGGCCACCCTCAGTTGT |
| | AGAGCCTCTCAGAGCGTCAACTCTAGATTTCTAGCCTGGTATCAGCAAAAGCCGGGCCAGGCACCA |
| | AGGTTGCTCATCTACGGCGCATCAACTAGGGCAACCGGGATTCCTGCCCGCTTCTCAGGCAGCGGG |
| | TCAGGGACTGAGTTTACACTTACGATCAGTAGCTTACAGAGCGAGGACTTTGCCGTCTACTATTGC |
| | ATGCAGGGAACTCACTGGCCATACACCTTCGGCCAAGGGACAAAAGTGGAAATCAAG |
| | (SEQ ID NO: 805) |
| TMPRSS4 | CAGGTCCAGCTAGTTCAGTCTGGGGCAGAAGTCAAGAAACCGGGTAGCTCAGTGAAGGTGTCCTGC |
| Ab67 scFv | AAAGCAAGCGGCTACACTTTCACCTCATATGATATTAATTGGGTGCGACAGGCACCAGGCCAAGGG |
| (VH-VL) | CTGGAATGGATGGGCGGCATCATTCCCATCTTCGGGACAGCCAATTACGCTCAGAAGTTTCAGGGA |
| | CGGGTTACGATTACTGCCGATGAAAGCACCTCTACTGCCTATATGGAGTTAAGCTCTCTGCGCAGC |
| | GAGGACACCGCTGTGTACTATTGTGCCACTACACCAGGCGATGCTTTCGACATATGGGGTCAGGGA |
| | ACAATGGTTACAGTCTCATCTGGAGGTGGAGGATCAGGAGGAGGCGGGTCAGGTGGCGGAGGCAGT |
| | GACATTGTAATGACCCAGTCTCCCGATAGCCTTGCCGTGTCCTTGGGCGAAAGGGCAACCATAAAT |
| | TGCAAGTCCTCCCAGTCCGTCTTGTACTCGAGTAACAACAAAAACTATCTCGCGTGGTATCAGCAA |
| | AAGCCCGGGCAACCTCCTAAACTGCTGATCTACTGGGCTAGTACCAGAGAGAGCGGCGTGCCAGAC |
| | CGTTTTTCTGGGAGTGGTAGCGGGACTGATTTCACCCTTACGATAAGTAGTCTCCAGGCCGAGGAC |
| | GTAGCGGTGTACTATTGTCAACAGTACAGTGACACACCTCTGACCTTTGGGCAAGGCACAAAAGTG |
| | GAAATCAAG (SEQ ID NO: 806) |
| TMPRSS4 | GAAGTGCAACTGCTCGAATCTGGCGGTGGGCTGGTCAAACCTGGTGGATCTCTTCGGCTGTCATGC |
| Ab68 scFv | GCAGCCTCCGGGTTTACCTTCTCCTCCTCCTGGATGCATTGGGTGCGACAGGCACCCGGGAAAGGC |
| (VH-VL) | CTGGAGTGGGTGTCAGCCATCGGGACTGCCGGTGATACCTATTACCCTGGCTCGGTGAAAGGGAGG |
| | TTCACCATTAGCCGGGATGACTCCAAGAATACGCTGTACTTGCAGATGAACTCACTCAAGACTGAG |
| | GACACGGCTGTCTATTACTGTGCCAGAGTGCGACTCGGCCACTTTGACCTGTGGGGACGCGGGACA |
| | CTTGTAACAGTCAGCTCAGGAGGAGGCGGCTCTGGAGGTGGCGGATCTGGTGGAGGCGGCAGTGAC |
| | ATCCAGATGACTCAGTCTCCGAGCAGCTTGAGTGCTTCTGTTGGGGATCGCGTTACCATTACATGC |
| | AGGGCCTCCCAGTCAATCTCTCGTTACTTAAATTGGTATCAGCAAAAACCCGGCAAGGCACCCAAG |
| | CTGCTGATTTACGCGGCGAGCAGTCTCCAAAGCGGAGTGCCATCCAGATTCAGCGGGAGCGGGTCG |
| | GGCACCGATTTTACCCTTACAATAAGTAGTTTGCAGCCAGAGGACTTCGCTACATACTATTGTCAA |
| | CAGTCCTATAGCAACCCACCTACTTTTGGCCAGGGAACTAAGCTAGAAATCAAG |
| | (SEQ ID NO: 807) |
| TMPRSS4 | GAAGTTCAACTGCTCGAGTCCGGCGGTGGGCTTGTTCAGCACGGCGGCTCTTTGCGGCTGTCCTGT |
| Ab69 scFv | GCTGCGAGCGGATTTGCCTTCTCCTCCTACGTCCTGCATTGGGTTCGGCAGGCACCTGGGAAGGGC |
| (VH-VL) | CTGGAGTGGGTGTCCAGTATCAGTTCCAGTAGCAGCTACATCTACTACGCCGACTCCGTGAAAGGG |
| | CGATTCACCATCTCACGCGACAATTCAAAAAACACACTGTACTTGCAGATGAACAGCCTTAGGGCC |
| | GAGGATACTGCTGTGTACTATTGCGCTCGTGGGGATCGCTATCCCGGCCTGCCCAATTATTGGGGT |
| | CAGGGAACATTAGTAACCGTGTCTAGTGGCGGAGGAGGAAGCGGCGGAGGCGGTAGCGGTGGAGGA |
| | GGATCTGACATTCAGATGACCCAGTCTCCAAGTAGCCTAAGTGCAAGCGTCGGCGACAGAGTAACA |
| | ATTACATGCAGAGCTTCGCAAGGGATCTCAAGTTGGCTCGCCTGGTACCAGCAGAAACCTGGGAAA |
| | GCACCTAAGCTTCTCATATACCAGGCCTCAAACAAGGATACTGGGGTGCCATCTAGGTTTTCTGGC |
| | TCTGGCTCGGGCACGGACTTCACCCTCACAATAAGCAGCTTACAGCCCGAAGATTTTGCCACGTAT |
| | TATTGTCAACAGTCATATAGGATTCCGTGGACTTTCGGGCAGGGAACCAAGGTGGAAATCAAG |
| | (SEQ ID NO: 808) |
| TMPRSS4 | GAGGTCCAGCTTCTGGAATCAGGCGGCGGGCTGGTCCAGCCTGGTGGTTCCTTACGGTTGTCCTGC |
| Ab70 scFv | GCTGCCTCCGGGTTTGCGTTTAGTGGCACTTGGATGCAGTGGGTGCGGCAGGCACCGGGCAAGGGC |
| (VH-VL) | CTGGAGTGGGTTTCTGACATTTCCGGGTCCAGTAGGGACACCAACTACGCTGACAGTGTAAAGGGA |
| | CGATTCACAATAAGCAGGGACAACTCTAAAAATACGCTGTACCTCCAGATGAACTCTCTGCGCGCT |
| | GAAGATACAGCCGTGTACTATTGTGCAAAAGATCACTGGGATTCATATGGGTATCTGGACTATTGG |
| | GGACAAGGGACTTTGGTGACAGTGTCTAGTGGAGGAGGAGGATCAGGCGGTGGCGGATCTGGCGGA |
| | GGCGGTTCCGACATTCAGATGACTCAGAGTCCAAGCAGCCTGAGCGCCAGCGTTGGCGATAGAGTG |
| | ACCATTACCTGCAGAGCGTCACAATCCATCTCGGGTTGGCTGGCCTGGTACCAGCAGAAGCCTGGG |
| | AAAGCACCCAAGCTCCTCATCTACGCAGCTTCGACCCTACGTGATGGGGTCCCATCTCGCTTTAGC |
| | GGGAGTGGCTCTGGCACTGATTTCACGCTCACAATAAGCAGCCTTCAGCCCGAGGACTTCGCCACC |
| | TACTATTGTCAACAGGCCAATTCCTTTCCCTTAACCTTCGGACAAGGGACAAAAGTGGAAATCAAG |
| | (SEQ ID NO: 809) |
| TMPRSS4 | GAAGTCCAACTGCTCGAGAGTGGCGGCGGGCTTGTTCAGCCTGGCGGTTCTTTGCGGCTGTCATGC |
| Ab71 scFv | GCCGCGAGCGGCTTCATGTTTGACTATTACGCCATGCATTGGGTTCGACAGGCTCCCGGGAAGGGC |
| (VH-VL) | CTAGAGTGGGTGTCCTTGATCTCCTATGATGGGAGGAACAAGTACTACGCCGACTCAGTGAAGGGC |
| | CGCTTCACAATCAGCCGGGATAATTCCAAAAACACCCTGTACCTCCAGATGAACAGCCTTAGAGCC |
| | GAAGATACCGCTGTCTACTATTGTGCAAGGCCCGGGAGCTATTCTAGATTTCAGCACTGGGGTCAG |
| | GGAACATTAGTGACAGTGTCGTCTGGAGGAGGTGGAAGCGGTGGCGGTGGATCTGGAGGAGGCGGG |
| | AGCGACATTCAGATGACCCAATCGCCAAGTTCCCTGAGTGCTTCAGTCGGCGACCGCGTAACTATT |
| | ACCTGTAGAGCCAGTCAAGGGATTTCTAATAACCTGAATTGGTATCAACAGAAGCCCGGGAAAGCT |
| | CCTAAACTGCTGATATACGCGGCATCCTCCCTCCAGTCAGGCGTGCCTAGTCGTTTTAGCGGGTCT |
| | GGCTCAGGCACTGACTTTACACTCACGATCAGCTCCTTACAGCCGGAGGATTTCGCCACTTATTAC |
| | TGCCAGCAGGCAAATAACTTCCCAATAACGTTCGGGCAGGGAACCAAAGTGGAAATCAAG |
| | (SEQ ID NO: 810) |
| TMPRSS4 | GAAGTGCAGCTGCTCGAGTCGGGCGGCGGGCTGGTGCAACCAGGAGGGTCATTACGGTTGTCATGC |
| Ab72 scFv | GCTGCCTCTGGGTTTACGTTCGGAGCCTACGTGATGCATTGGGTCCGGCAGGCACCTGGGAAAGGC |
| (VH-VL) | CTGGAGTGGGTGTCCTCCATTAGTGGCGGCAGCACTTACTACGCCGACTCCGTTAAAGGGAGATTC |
| | ACAATAAGCAGGGACAACTCAAAGAACACGCTGTACCTCCAGATGAATAGCCTTCGCGCAGAAGAT |
| | ACCGCCGTGTACTATTGCGCCAGGCACCCAGTCCGTGGCGTGATTGGGGCAGGCTGGTTCGATCCT |
| | TGGGGTCAGGGTACACTTGTAACAGTGTCCAGTGGAGGAGGTGGAAGTGGTGGCGGAGGTTCTGGA |
| | GGAGGAGGGTCCGACATACAGATGACTCAGTCTCCAAGCTCCTTGTCAGCTTCTGTTGGGGACCGA |
| | GTTACCATCACTTGTAGGGCCTCACAAAATATCTCCCGCTGGCTGGCTTGGTATCAGCAAAAGCCG |
| | GGCAAAGCTCCCAAGCTGCTGATCTACGCGGCGAGTAGCCTCCAGTCGGGAGTACCCAGCAGATTT |
| | AGCGGCTCCGGGTCTGGGACCGATTTCACCCTAACAATCAGTAGCCTCCAGCCCGAGGATTTTGCC |
| | ACTTATTATTGTCAACAGGCAATTAGCTTCCCTTTAACCTTTGGCGGTGGCACCAAGGTCGAAATC |
| | AAG (SEQ ID NO: 811) |
| TMPRSS4 | GAGGTTCAGCTTCTCGAGTCGGGCGGAGGGCTAGTTCAGCCTGGCGGGTCTTTGCGTCTGAGCTGT |
| Ab73 scFv | GCCGCCAGCGGCTTTACCTTCTCCTCATACGCCATGCATTGGGTTCGACAGGCTCCTGGGAAGGGC |
| (VH-VL) | CTGGAGTGGCTCGCCGTAATCTCCTTCGACGGGTCTATCAGACACTATGCGGACTCCGTGAAAGGC |
| | AGGTTCACCATTTCTCGGGACAATTCCAAGAACACGCTGTACTTGCAGATGAACAGTCTGCGGGCA |
| | GAAGATACCGCCGTGTACTATTGCGCCAAACCAAAGGCCTCCAGCGGGCCGCGCTTGATAGATTAC |
| | TGGGGACAAGGGACTCTTGTGACCGTCAGCTCAGGTGGAGGAGGAAGTGGCGGTGGTGGGTCTGGT |
| | GGTGGCGGGAGCGACATTCAGATGACACAGAGTCCATCTTCACTGTCAGCTTCGGTCGGCGACAGA |
| | GTGACTATTACATGTCGAGCATCCCAAGGAATCTCCAATTCTCTGGCGTGGTATCAACAGAAACCC |
| | GGCAAAGCTCCCAAGCTGCTGATATACAGCGCAGTGAACCTCCAGAGCGGAGTGCCCTCACGCTTT |
| | TCCGGAAGTGGCTCTGGGACTGATTTTACACTTACGATAAGTAGCCTCCAGCCTGAAGATTTCGCT |
| | ACATATTACTGCCAACAGGCAAATAGCTTTCCATTAACTTTCGGCGGCGGAACAAAAGTCGAAATC |
| | AAG (SEQ ID NO: 812) |
| TMPRSS4 | GAAGTGCAGCTGCTCGAAAGTGGAGGCGGGCTGGTTCAACCCGGAGGGAGCTTGCGGCTCTCATGC |
| Ab74 scFv | GCTGCGAGCGGTTTTACCTTCTCCTCCTATGCCATGCATTGGGTTCGGCAGGCGCCCGGGAAAGGC |
| (VH-VL) | CTGGAGTGGGTGTCCTCCATCTCAAGTAGCTCTACCTACATTCACTACGCCGATAGCGTCAAGGGT |
| | AGATTCACGATCTCCCGCGACAACAGCAAAAATACCCTGTACCTCCAGATGAACTCCTTACGAGCC |
| | GAGGACACTGCTGTGTACTATTGTGCAAGGGTGGGGAGGTATTACGGCAGCGGCTCATCACTGGTA |
| | GACTATTGGGGACAAGGGACACTTGTCACCGTGTCTAGCGGCGGCGGTGGATCAGGTGGCGGAGGA |
| | AGTGGAGGTGGAGGGTCTGACATTCAGATGACTCAGTCGCCATCATCGTTGTCCGCTAGTGTCGGC |
| | GATAGAGTGACTATTACCTGCCGTGCCTCTCAGAGCGTGTCCTCCTGGCTGGCATGGTATCAACAG |
| | AAACCTGGGAAGGCACCGAAGCTGCTCATCTACGATGCCTCTAGTCTACAGTCTGGCGTACCCTCC |
| | CGCTTTAGCGGCTCTGGGTCTGGCACCGATTTCACTCTTACAATAAGCAGTTTACAGCCTGAGGAC |
| | TTCGCCACATATTACTGTCAACAGGCTAAAAGTTTTCCACCTACGTTTGGCCAAGGGACAAAGGTC |
| | GAAATCAAG (SEQ ID NO: 813) |
| TMPRSS4 | GAAGTGCAGTTACTCGAGAGCGGCGGCGGATTGGTCCAGCCTGGCGGTAGCCTGCGGTTGTCATGC |
| Ab75 scFv | GCTGCGAGTGGGTTCACCTTCTCCTCATACGCCATGTCGTGGGTCCGGCAGGCACCCGGCAAAGGC |
| (VH-VL) | CTGGAGTGGGTGTCCTCCATCAGTAGTGCTTCTAGCTACAAGTATTATGCCGACTCCGTGAAAGGG |
| | CGATTTACCATCTCCCGAGACAATTCCAAGAATACGCTGTACCTCCAGATGAACTCTCTTCGCGCT |
| | GAAGATACCGCCGTGTACTATTGTGCAAGGGATATATACTCCAGCGGATGGCGCGGCTACTACTAC |
| | TATGGGATGGATGTTTGGGGACAAGGGACCACGGTGACAGTGTCAAGTGGAGGCGGCGGTTCTGGA |
| | GGAGGCGGATCTGGAGGCGGCGGTTCAGACATTCAGATGACCCAGTCTCCATCTTCACTGTCAGCT |
| | AGTGTTGGGGATAGAGTCACTATAACTTGCAGGGCCTCTCAAGGGATTAGGAACGACCTTAACTGG |
| | TATCAGCAAAAGCCCGGGAAAGCACCGAAGCTCCTGATTTACGCAGCGACAAGACTGCAAAGCGGC |
| | GTACCTAGCAGATTCAGCGGGTCGGGTTCCGGGACTGATTTTACCCTCACAATCAGCTCCCTACAG |
| | CCAGAGGACTTCGCCACTTATTACTGTCAACAGGCCCATAGTTTTCCCTATAGCTTTGGGCAGGGA |
| | ACACGTCTGGAAATCAAG (SEQ ID NO: 814) |
| TMPRSS4 | GAAGTCCAACTCCTGGAGAGTGGCGGCGGGCTGGTACAGCCCGGCGGATCTTTGCGGCTGAGTTGT |
| Ab76 scFv | GCTGCAAGCGGGTTTACCTTCTCCTCATATGCCATGTCATGGGTTCGGCAGGCACCTGGGAAGGGC |
| (VH-VL) | CTAGAGTGGGTCTCAGCCATTAGCGGGTCGGGTGGCAATGCGTACTATGCGGACAGCGTGAAGGGC |
| | CGTTTCACCATCAGCCGCGACAATGCCAAAAACAGTCTCTACTTGCAGATGAACTCCCTGAGGGCT |
| | GAGGACACTGCGGTGTACTATTGCGCCAAAAATAGTTGGGGCTCTTATAGGCCAAGAGCCTTTGAT |
| | ATTTGGGGACAAGGGACAATGGTGACTGTGTCCTCCGGCGGAGGTGGATCAGGTGGCGGAGGAAGC |
| | GGAGGTGGAGGGAGTGACATTGTGATGACACAAAGCCCAGATTCTCTTGCTGTCAGCCTCGGAGAG |
| | AGAGCCACGATCAATTGCAAATCCTCCCAGTCTGTGCTGTACTCATCTAACAACAAGAACTATCTC |
| | GCATGGTACCAGCAGAAACCGGGCCAGCCTCCCAAACTGCTGATCTACTGGGCCTCTACCCGAGCA |
| | TCGGGCGTGCCTGACCGCTTTAGCGGGTCTGGGTCTGGGACCGATTTCACTCTTACAATAAGTTCC |
| | TTACAAGCAGAAGATGTCGCCGTTTATTACTGCCAGCAGTATCTGAGCTTACCCTACACCTTCGGC |
| | CAGGGTACAAAAGTTGAAATCAAG (SEQ ID NO: 815) |
| TMPRSS4 | CAGGTTCAGCTTGTCCAGTCTGGTGCCGAGGTGAAAAAGCCTGGCGCGTCAGTTAAAGTGTCCTGC |
| Ab77 scFv | AAGGCCAGCGGGAATATCTTCACCGCACAGTACATGCATTGGGTTCGGCAGGCACCAGGCCAGGGT |
| (VH-VL) | TTGGAATGGATGGGCTGGATGAATCCGAATACCGTCTATACCGGGAGTGCCCAAAAGTTCCAGGGC |
| | CGAGTCACTATGACAAGAGACACCAGCACATCCACTGTCTACATGGAGCTAAGCTCTCTGCGCTCA |
| | GAAGATACCGCTGTGTACTATTGCGCCAGGGATTGGGTCGGTGATGGGTATAATAGCTTTGATTAT |
| | TGGGGACAAGGCACATTAGTAACTGTGTCCAGTGGCGGAGGCGGCTCCGGAGGAGGAGGTAGTGGA |
| | GGAGGAGGGTCAGACATTGTTATGACCCAGTCACCAGATTCGCTGGCAGTGTCCCTGGGCGAAAGG |
| | GCTACTATTAACTGTAAAAGTAGCCAGTCCGTGCTCTATAGCAGTAACAACAAAAACTACTTGGCC |
| | TGGTACCAACAGAAGCCCGGGCAGCCTCCCAAACTGCTGATCTACTGGGCCTCTACAAGAGAGTCT |
| | GGGGTGCCCGACCGTTTTTCTGGCTCTGGGTCCGGCACTGACTTCACGCTTACGATAAGCAGCCTC |
| | CAGGCTGAGGACGTAGCTGTGTACTATTGTCAACAGTACTATACCACACCTTTCACGTTTGGCCCA |
| | GGGACAAAGGTGGACATCAAG (SEQ ID NO: 816) |
| TMPRSS4 | GAAGTTCAGCTGCTCGAAAGCGGAGGCGGGCTGGTCCAGCCTGGCGGGAGTTTACGGTTGTCCTGT |
| Ab78 scFv | GCCGCGTCTGGTTTTACCTTCTCCTCATACGGCATGAATTGGGTGCGCCAGGCACCCGGGAAAGGC |
| (VH-VL) | CTGGAGTGGGTCAGCGCTATTAGTGGTTCTGGTGGCAGGACTTACTACGCTGATTCAGTTAAAGGG |
| | AGATTCACCATTTCACGAGACAACGCTAAAAACAGCCTGTACTTGCAGATGAACTCCCTTAGGGCC |
| | GAGGACACCGCAGTGTATTACTGCGCTAAGGGCACATATTATTCCTCGCCAAAGTATTCGTTTGAC |
| | TATTGGGGACAAGGCACTCTCGTAACCGTGTCCTCCGGCGGAGGAGGTTCCGGAGGAGGTGGATCA |
| | GGCGGTGGAGGGAGTGACATCCAGATGACGCAGAGTCCTAGCTCCCTGTCAGCCTCTGTCGGCGAT |
| | AGAGTGACAATTACTTGCCGGGCGAGTCAAGATATCAAGAATTTCCTCGCATGGTATCAGCAAAAG |
| | CCCGGGAAAGCACCGAAACTGCTGATCTACGCCGCCAGCAGCCTCCAGAGCGGCGTGCCATCTCGT |
| | TTTTCAGGCTCTGGGTCTGGGACAGATTTCACACTTACCATAAGTAGCTTACAGCCCGAGGACTTT |
| | GCCACCTATTACTGTCAACAGTCTTACAGCACTCCTTGGACGTTCGGGCAGGGAACAAAGCTAGAA |
| | ATCAAG (SEQ ID NO: 817) |
| TMPRSS4 | GAAGTTCAGCTTCTCGAGAGCGGCGGCGGGCTGGTGCAACCGGGAGGTTCTTTGCGGCTGTCATGT |
| Ab79 scFv | GCCGCGAGCGGGCTGACCTTCAGCTCATATCAGATGTCATCCGTCAGTCAGGCACCTGGCAAAGGC |
| (VH-VL) | CTGGAGTGGGTGTCCTACATAAGCAGTGCAGCCAATACTGTGTACTATGCGGACAGCGTTAAGGGC |
| | CGATTCACGATCAGTCGGGACAACTCCAAGAACACTCTGTACCTCCAGATGAACTCCTTACGCGCA |
| | GAGGATACTGCTGTGTACTATTGCGCCAGGGAAGATGAGTCTAGATCGCCTTATTGTAGCGGCGGG |
| | TCTTGCTACCGTGCTGAATACTTTCAACATTGGGGTCAGGGTACACTTGTAACCGTGTCCTCCGGA |
| | GGCGGAGGGAGTGGAGGCGGTGGTTCTGGAGGAGGAGGCTCTGACATTCAGATGACACAGAGCCCA |
| | AGCTCACTGTCAGCTTCTGTAGGCGACCGCGTCACCATCACTTGCAGAGCCTCGCAGGGAATTTCT |
| | AATTACCTAGCATGGTATCAACAGAAGCCAGGGAAAGCTCCTAAGCTGCTGATCTACGCAGCAAGT |
| | AGTCTCCAGAGTGGAGTGCCGTCCAGGTTTAGCGGCAGCGGTTCAGGGACGGACTTCACCCTCACC |
| | ATAAGTTCCTTACAGCCCGAGGATTTCGCCACCTATTATTGTCAACAGAGCTACTCTACACCCTTG |
| | ACATTTGGGCCAGGGACAAAAGTCGATATTAAG (SEQ ID NO: 818) |

In some embodiments, the antibody or antigen-binding fragment that binds to TMPRSS4 comprises an scFv encoded by a nucleic acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the nucleic acid sequence set forth in SEQ ID NO: 740, and optionally comprises nucleic acid sequences encoding VH CDRs and VL CDRs that are 100% identical to those therein. In some embodiments, the antibody or antigen-binding fragment that binds to TMPRSS4 comprises an scFv encoded by the nucleic acid sequence set forth in any one of SEQ ID NOs: 740-818, and optionally comprises VH CDRs and VL CDRs that are 100% identical to those therein.

### SLC34A2 Antigen Binding Domains

In some aspects, provided herein are antigen binding domains (e.g., antibodies or antigen binding fragments thereof) that bind to SLC34A2. In some aspects, provided herein are means for binding to SLC34A2. In some embodiments, the means for binding to SLC34A2 comprises an antibody or antigen-binding fragment provided herein. In some embodiments, a SLC34A2 antibody or antigen-binding fragment or equivalent thereof comprises means for binding a SLC34A2 protein, optionally binding a human SLC34A2 protein in the region(s) of human SLC34A2 bound by the SLC34A2 antigen binding domains (e.g., an antibody or antigen binding fragment thereof as described in the Examples below). In some embodiments, the means binds a SLC34A2 protein. In some embodiments, the means binds a human SLC34A2 protein (e.g., the SLC34A2 protein of SEQ ID NO: 962) and related isoforms and orthologs. In some embodiments, the means is a SLC34A2 antibody or antigen-binding fragment or equivalent thereof (e.g., a full length antibody or a F(ab′)₂ fragment, a Fab fragment, a single chain variable fragment (scFv), and a single domain antibody (sdAb), or a functional fragment thereof) means for binding a SLC34A2 protein. In some embodiments, the means for binding SLC34A2 includes the anti-SLC34A2 antibodies and antigen-binding fragments or equivalents thereof described herein.

Solute Carrier Family 34 Member 2 (SLC34A2 HGNC: 11020, NCBI Entrez Gene: 10568; UniProtKB/Swiss-Prot: 095436), otherwise known as NaPi2b, NPT2B, NPTIIb, and Sodium-Phosphate Transport Protein 2B, is a pH-sensitive sodium-dependent phosphate transporter involved in actively transporting phosphate into cells via Na(+) cotransport. Phosphate uptake via SLC34A2 is increased at a lower pH.

The amino acid and nucleic acid sequences of human SLC34A2 are provided below in Table 8.

TABLE 8
SLC34A2 sequences

| | |
|---|---|
| Human | MAPWPELGDAQPNPDKYLEGAAGQQPTAPDKSKETNKTDNTEAPVTKIELLPSYSTATLIDEPTEV |
| SLC34A2 | DDPWNLPTLQDSGIKWSERDTKGKILCFFQGIGRLILLLGFLYFFVCSLDILSSAFQLVGGKMAGQ |
| SEQ ID | FFSNSSIMSNPLLGLVIGVLVTVLVQSSSTSTSIVVSMVSSSLLTVRAAIPIIMGANIGTSITNTI |
| NO: 962 | VALMQVGDRSEFRRAFAGATVHDFFNWLSVLVLLPVEVATHYLEIITQLIVESFHFKNGEDAPDLL |
| | KVITKPFTKLIVQLDKKVISQIAMNDEKAKNKSLVKIWCKTFTNKTQINVTVPSTANCTSPSLCWT |
| | DGIQNWTMKNVTYKENIAKCQHIFVNFHLPDLAVGTILLILSLLVLCGCLIMIVKILGSVLKGQVA |
| | TVIKKTINTDFPFPFAWLTGYLAILVGAGMTFIVQSSSVFTSALTPLIGIGVITIERAYPLTLGSN |
| | IGTTTTAILAALASPGNALRSSLQIALCHFFFNISGILLWYPIPFTRLPIRMAKGLGNISAKYRWF |
| | AVFYLIIFFFLIPLTVFGLSLAGWRVLVGVGVPVVFIIILVLCLRLLQSRCPRVLPKKLQNWNFLP |
| | LWMRSLKPWDAVVSKFTGCFQMRCCCCCRVCCRACCLLCDCPKCCRCSKCCEDLEEAQEGQDVPVK |
| | APETFDNITISREAQGEVPASDSKTECTAL |
| Human | ATGGCTCCCTGGCCTGAATTGGGAGATGCCCAGCCCAACCCCGATAAGTACCTCGAAGGGGCCGCA |
| SLC34A2 | GGTCAGCAGCCCACTGCCCCTGATAAAAGCAAAGAGACCAACAAAACAGATAACACTGAGGCACCT |
| SEQ ID | GTAACCAAGATTGAACTTCTGCCGTCCTACTCCACGGCTACACTGATAGATGAGCCCACTGAGGTG |
| NO: 963 | GATGACCCCTGGAACCTACCCACTCTTCAGGACTCGGGGATCAAGTGGTCAGAGAGAGACACCAAA |
| | GGGAAGATTCTCTGTTTCTTCCAAGGGATTGGGAGATTGATTTTACTTCTCGGATTTCTCTACTTT |
| | TTCGTGTGCTCCCTGGATATTCTTAGTAGCGCCTTCCAGCTGGTTGGAGGAAAAATGGCAGGACAG |
| | TTCTTCAGCAACAGCTCTATTATGTCCAACCCTTTGTTGGGGCTGGTGATCGGGGTGCTGGTGACC |
| | GTCTTGGTGCAGAGCTCCAGCACCTCAACGTCCATCGTTGTCAGCATGGTGTCCTCTTCATTGCTC |
| | ACTGTTCGGGCTGCCATCCCCATTATCATGGGGGCCAACATTGGAACGTCAATCACCAACACTATT |
| | GTTGCGCTCATGCAGGTGGGAGATCGGAGTGAGTTCAGAAGAGCTTTTGCAGGAGCCACTGTCCAT |
| | GACTTCTTCAACTGGCTGTCCGTGTTGGTGCTCTTGCCCGTGGAGGTGGCCACCCATTACCTCGAG |
| | ATCATAACCCAGCTTATAGTGGAGAGCTTCCACTTCAAGAATGGAGAAGATGCCCCAGATCTTCTG |
| | AAAGTCATCACTAAGCCCTTCACAAAGCTCATTGTCCAGCTGGATAAAAAAGTTATCAGCCAAATT |
| | GCAATGAACGATGAAAAAGCGAAAAACAAGAGTCTTGTCAAGATTTGGTGCAAAACTTTTACCAAC |
| | AAGACCCAGATTAACGTCACTGTTCCCTCGACTGCTAACTGCACCTCCCCTTCCCTCTGTTGGACG |
| | GATGGCATCCAAAACTGGACCATGAAGAATGTGACCTACAAGGAGAACATCGCCAAATGCCAGCAT |
| | ATCTTTGTGAATTTCCACCTCCCGGATCTTGCTGTGGGCACCATCTTGCTCATACTCTCCCTGCTG |
| | GTCCTCTGTGGTTGCCTGATCATGATTGTCAAGATCCTGGGCTCTGTGCTCAAGGGGCAGGTCGCC |
| | ACTGTCATCAAGAAGACCATCAACACTGATTTCCCCTTTCCCTTTGCATGGTTGACTGGCTACCTG |
| | GCCATCCTCGTCGGGGCAGGCATGACCTTCATCGTACAGAGCAGCTCTGTGTTCACGTCGGCCTTG |
| | ACCCCCCTGATTGGAATCGGCGTGATAACCATTGAGAGGGCTTATCCACTCACGCTGGGCTCCAAC |
| | ATCGGCACCACCACCACCGCCATCCTGGCCGCCTTAGCCAGCCCTGGCAATGCATTGAGGAGTTCA |
| | CTCCAGATCGCCCTGTGCCACTTTTTCTTCAACATCTCCGGCATCTTGCTGTGGTACCCGATCCCG |
| | TTCACTCGCCTGCCCATCCGCATGGCCAAGGGGCTGGGCAACATCTCTGCCAAGTATCGCTGGTTC |
| | GCCGTCTTCTACCTGATCATCTTCTTCTTCCTGATCCCGCTGACGGTGTTTGGCCTCTCGCTGGCC |
| | GGCTGGCGGGTGCTGGTTGGTGTCGGGGTTCCCGTCGTCTTCATCATCATCCTGGTACTGTGCCTC |
| | CGACTCCTGCAGTCTCGCTGCCCACGCGTCCTGCCGAAGAAACTCCAGAACTGGAACTTCCTGCCG |
| | CTGTGGATGCGCTCGCTGAAGCCCTGGGATGCCGTCGTCTCCAAGTTCACCGGCTGCTTCCAGATG |
| | CGCTGCTGCTGCTGCTGCCGCGTGTGCTGCCGCGCGTGCTGCTTGCTGTGTGACTGCCCCAAGTGC |
| | TGCCGCTGCAGCAAGTGCTGCGAGGACTTGGAGGAGGCGCAGGAGGGGCAGGATGTCCCTGTCAAG |
| | GCTCCTGAGACCTTTGATAACATAACCATTAGCAGAGAGGCTCAGGGTGAGGTCCCTGCCTCGGAC |
| | TCAAAGACCGAATGCACGGCCTTG |

In some embodiments, the SLC34A2 antigen-binding moiety (e.g., an antigen binding protein or domain such as an antibody of antigen binding fragment thereof) is selected from the group consisting of an antibody, a nanobody, a diabody, a triabody, or a minibody, a F(ab′)₂ fragment, a Fab fragment, a single chain variable fragment (scFv), and a single domain antibody (sdAb), or a functional fragment thereof. In some embodiments, the antigen-binding moiety comprises an scFv. The antigen-binding moiety can include naturally-occurring amino acid sequences or can be engineered, designed, or modified so as to provide desired and/or improved properties, e.g., increased binding affinity.

In some embodiments, provided SLC34A2 antigen-binding moieties, including antigen-binding fragments thereof, include any combination of the heavy chain and light chain complementarity-determining regions (CDRs) described herein. In some embodiments, the anti-SLC34A2 antibody or antigen-binding fragment thereof comprises any one of the CDR-H1 as described herein, any one of the CDR-H2 as described herein, any one of the CDR-H3 as described herein, any one of the CDR-L1 as described herein, any one of the CDR-L2 as described herein and any one of the CDR-L3 as described herein. In some of any such embodiments, any one or more of the CDR-H1, the CDR-H2 and the CDR-H3 sequences described herein, and any one or more of the CDR-L1, the CDR-L2 and the CDR-L3 sequences described herein can be used in combination.

Also among the antibodies are those having sequences at least at or about 90%, at or about 91%, at or about 92%, at or about 93%, at or about 94%, at or about 95%, at or about 96%, at or about 97%, at or about 98%, or at or about 99% identical to any such CDR sequence, e.g., any of the CDR-H1, CDR-H2, CDR-H3, CDR-L1, CDR-L2, CDR-L3. In some embodiments, among the antibodies are those in which a CDR contained therein has no more than 2 amino acid difference compared to any such above CDR sequence, e.g., any of the CDR-H1, CDR-H2, CDR-H3, CDR-L1, CDR-L2, CDR-L3. In some embodiments, among the antibodies are those in which a CDR contained therein has no more than 1 amino acid difference compared to any such above CDR sequence, e.g., any of the CDR-H1, CDR-H2, CDR-H3, CDR-L1, CDR-L2, CDR-L3.

In some embodiments, a provided anti-SLC34A2 antibody or an antigen-binding fragment thereof has a CDR-H1, a CDR-H2 and a CDR-H3 present in a VH region amino acid sequence set forth in any one of SEQ ID NOs: 1001, 1009, 1015, 1023, 1031, 1039, 1047, 1053, 1059, 1066, 1073, 1078, 1084, 1090, 1094, 1100, or an amino acid sequence that has at least at or about 85%, 86%, 87%, 88%, 89%, 90%, 91%, 92%, 93%, 94%, 95%, 96%, 97%, 98%, or 99% sequence identity to the VH region amino acid sequence set forth in any one of SEQ ID NOs: 1001, 1009, 1015, 1023, 1031, 1039, 1047, 1053, 1059, 1066, 1073, 1078, 1084, 1090, 1094, 1100, and a CDR-L1, a CDR-L2 and a CDR-L3 present in a VL region amino acid sequence set forth in any one of SEQ ID NOs: 1005, 1013, 1019, 1027, 1035, 1043, 1051, 1056, 1063, 1070, 1076, 1082, 1088, 1093, 1097, 1103, 1125, 1154, 1155, 1156, 1178, 1233, or 1234, or an amino acid sequence that has at least at or about 85%, 86%, 87%, 88%, 89%, 90%, 91%, 92%, 93%, 94%, 95%, 96%, 97%, 98%, or 99% sequence identity to the VL region amino acid sequence set forth in any one of SEQ ID NOs: 1005, 1013, 1019, 1027, 1035, 1043, 1051, 1056, 1063, 1070, 1076, 1082, 1088, 1093, 1097, 1103, 1154, 1125, 1155, 1156, 1178, 1233, or 1234.

In some embodiments, a provided anti-SLC34A2 antibody or an antigen-binding fragment thereof has a CDR-H1, a CDR-H2 and a CDR-H3 present in a VH region amino acid sequence set forth in any one of SEQ ID NOs: 1001, 1009, 1015, 1023, 1031, 1039, 1047, 1053, 1059, 1066, 1073, 1078, 1084, 1090, 1094, 1100, and a CDR-L1, a CDR-L2 and a CDR-L3 present in a VL region amino acid sequence set forth in any one of SEQ ID NOs: 1005, 1013, 1019, 1027, 1035, 1043, 1051, 1056, 1063, 1070, 1076, 1082, 1088, 1093, 1097, 1103, 1125, 1154, 1155, 1156, 1178, 1233, or 1234. In some embodiments, the combination of six CDRs (a CDR-H1, a CDR-H2, a CDR-H3, a CDR-L1, a CDR-L2 and a CDR-L3) is according to Kabat, Chothia, AbM, IMGT, or Contact numbering.

Exemplary heavy and light chain CDR sequences of the anti-SLC34A2 antibodies or antigen-binding fragments thereof are provided in Table 9 provided herein.

In some of any of the provided embodiments, the VH region contains a CDR-H1 set forth in SEQ ID NO: 1002, a CDR-H2 set forth in SEQ ID NO: 1003, and a CDR-H3 set forth in SEQ ID NO: 1004; and the VL region contains a CDR-L1 set forth in SEQ ID NO: 1006, a CDR-L2 set forth in SEQ ID NO: 1007, and a CDR-L3 set forth in SEQ ID NO: 1008. In some of any of the provided embodiments, the VH region contains a CDR-H1 set forth in SEQ ID NO: 1010, a CDR-H2 set forth in SEQ ID NO: 1011, and a CDR-H3 set forth in SEQ ID NO: 1012; and the VL region contains a CDR-L1 set forth in SEQ ID NO: 1006, a CDR-L2 set forth in SEQ ID NO: 1007, and a CDR-L3 set forth in SEQ ID NO: 1014. In some of any of the provided embodiments, the VH region contains a CDR-H1 set forth in SEQ ID NO: 1016, a CDR-H2 set forth in SEQ ID NO: 1017, and a CDR-H3 set forth in SEQ ID NO: 1018; and the V_{L} region contains a CDR-L1 set forth in SEQ ID NO: 1020, a CDR-L2 set forth in SEQ ID NO: 1021, and a CDR-L3 set forth in SEQ ID NO: 1022. In some of any of the provided embodiments, the V_{H} region contains a CDR-H1 set forth in SEQ ID NO: 1024, a CDR-H2 set forth in SEQ ID NO: 1025, and a CDR-H3 set forth in SEQ ID NO: 1026; and the V_{L} region contains a CDR-L1 set forth in SEQ ID NO: 1028, a CDR-L2 set forth in SEQ ID NO: 1029, and a CDR-L3 set forth in SEQ ID NO: 1030. In some of any of the provided embodiments, the V_{H} region contains a CDR-H1 set forth in SEQ ID NO: 1032, a CDR-H2 set forth in SEQ ID NO: 10 33, and a CDR-H3 set forth in SEQ ID NO: 1034; and the V_{L} region contains a CDR-L1 set forth in SEQ ID NO: 1036, a CDR-L2 set forth in SEQ ID NO: 1037, and a CDR-L3 set forth in SEQ ID NO: 1038. In some of any of the provided embodiments, the V_{H} region contains a CDR-H1 set forth in SEQ ID NO: 1040, a CDR-H2 set forth in SEQ ID NO: 1041, and a CDR-H3 set forth in SEQ ID NO: 1042; and the V_{L} region contains a CDR-L1 set forth in SEQ ID NO: 1044, a CDR-L2 set forth in SEQ ID NO: 1045, and a CDR-L3 set forth in SEQ ID NO: 1046. In some of any of the provided embodiments, the V_{H} region contains a CDR-H1 set forth in SEQ ID NO: 1048, a CDR-H2 set forth in SEQ ID NO: 1049, and a CDR-H3 set forth in SEQ ID NO: 1050; and the V_{L} region contains a CDR-L1 set forth in SEQ ID NO: 1036, a CDR-L2 set forth in SEQ ID NO: 1021, and a CDR-L3 set forth in SEQ ID NO: 1052. In some of any of the provided embodiments, the V_{H} region contains a CDR-H1 set forth in SEQ ID NO: 1048, a CDR-H2 set forth in SEQ ID NO: 1054, and a CDR-H3 set forth in SEQ ID NO: 1055; and the V_{L} region contains a CDR-L1 set forth in SEQ ID NO: 1036, a CDR-L2 set forth in SEQ ID NO: 1057, and a CDR-L3 set forth in SEQ ID NO: 1058. In some of any of the provided embodiments, the V_{H} region contains a CDR-H1 set forth in SEQ ID NO: 1060, a CDR-H2 set forth in SEQ ID NO: 1061, and a CDR-H3 set forth in SEQ ID NO: 1062; and the V_{L} region contains a CDR-L1 set forth in SEQ ID NO: 1064, a CDR-L2 set forth in SEQ ID NO: 1021, and a CDR-L3 set forth in SEQ ID NO: 1065. In some of any of the provided embodiments, the V_{H} region contains a CDR-H1 set forth in SEQ ID NO: 1067, a CDR-H2 set forth in SEQ ID NO: 1068, and a CDR-H3 set forth in SEQ ID NO: 1069; and the V_{L} region contains a CDR-L1 set forth in SEQ ID NO: 1064, a CDR-L2 set forth in SEQ ID NO: 1021, and a CDR-L3 set forth in SEQ ID NO: 1065. In some of any of the provided embodiments, the V_{H} region contains a CDR-H1 set forth in SEQ ID NO: 1067, a CDR-H2 set forth in SEQ ID NO: 1068, and a CDR-H3 set forth in SEQ ID NO: 1069; and the V_{L} region contains a CDR-L1 set forth in SEQ ID NO: 1028, a CDR-L2 set forth in SEQ ID NO: 1071, and a CDR-L3 set forth in SEQ ID NO: 1072. In some of any of the provided embodiments, the V_{H} region contains a CDR-H1 set forth in SEQ ID NO: 1032, a CDR-H2 set forth in SEQ ID NO: 1074, and a CDR-H3 set forth in SEQ ID NO: 1075; and the V_{L} region contains a CDR-L1 set forth in SEQ ID NO: 1036, a CDR-L2 set forth in SEQ ID NO: 1021, and a CDR-L3 set forth in SEQ ID NO: 1077. In some of any of the provided embodiments, the V_{H} region contains a CDR-H1 set forth in SEQ ID NO: 1079, a CDR-H2 set forth in SEQ ID NO: 1080, and a CDR-H3 set forth in SEQ ID NO: 1081; and the V_{L} region contains a CDR-L1 set forth in SEQ ID NO: 1036, a CDR-L2 set forth in SEQ ID NO: 1021, and a CDR-L3 set forth in SEQ ID NO: 1083. In some of any of the provided embodiments, the V_{H} region contains a CDR-H1 set forth in SEQ ID NO: 1085, a CDR-H2 set forth in SEQ ID NO: 1086, and a CDR-H3 set forth in SEQ ID NO: 1087; and the V_{L} region contains a CDR-L1 set forth in SEQ ID NO: 1089, a CDR-L2 set forth in SEQ ID NO: 1021, and a CDR-L3 set forth in SEQ ID NO: 1065. In some of any of the provided embodiments, the V_{H} region contains a CDR-H1 set forth in SEQ ID NO: 1010, a CDR-H2 set forth in SEQ ID NO: 1091, and a CDR-H3 set forth in SEQ ID NO: 1092; and the V_{L} region contains a CDR-L1 set forth in SEQ ID NO: 106, a CDR-L2 set forth in SEQ ID NO: 107, and a CDR-L3 set forth in SEQ ID NO: 108. In some of any of the provided embodiments, the V_{H} region contains a CDR-H1 set forth in SEQ ID NO: 1095, a CDR-H2 set forth in SEQ ID NO: 103, and a CDR-H3 set forth in SEQ ID NO: 1096; and the V_{L} region contains a CDR-L1 set forth in SEQ ID NO: 1036, a CDR-L2 set forth in SEQ ID NO: 1098, and a CDR-L3 set forth in SEQ ID NO: 1099. In some of any of the provided embodiments, the V_{H} region contains a CDR-H1 set forth in SEQ ID NO: 1101, a CDR-H2 set forth in SEQ ID NO: 1102, and a CDR-H3 set forth in SEQ ID NO: 1096; and the V_{L} region contains a CDR-L1 set forth in SEQ ID NO: 1036, a CDR-L2 set forth in SEQ ID NO: 1104, and a CDR-L3 set forth in SEQ ID NO: 1105. In some of any of the provided embodiments, the V_{H} region contains a CDR-H1 set forth in SEQ ID NO: 1048, a CDR-H2 set forth in SEQ ID NO: 1049, and a CDR-H3 set forth in SEQ ID NO: 1050; and the V_{L} region contains a CDR-L1 set forth in SEQ ID NO: 1036, a CDR-L2 set forth in SEQ ID NO: 15, and a CDR-L3 set forth in SEQ ID NO: 1052. In some of any of the provided embodiments, the V_{H} region contains a CDR-H1 set forth in SEQ ID NO: 1032, a CDR-H2 set forth in SEQ ID NO: 1074, and a CDR-H3 set forth in SEQ ID NO: 1075; and the V_{L} region contains a CDR-L1 set forth in SEQ ID NO: 1251, a CDR-L2 set forth in SEQ ID NO: 15, and a CDR-L3 set forth in SEQ ID NO: 1077.

In some embodiments, any of the provided anti-SLC34A2 antibodies or antigen binding fragments has a V_{H} region having the amino acid sequence set forth in any one of SEQ ID NOS: 1001, 1009, 1015, 1023, 1031, 1039, 1047, 1053, 1059, 1066, 1073, 1078, 1084, 1090, 1094, 1100, or an amino acid sequence that has at least at or about 85%, 86%, 87%, 88%, 89%, 90%, 91%, 92%, 93%, 94%, 95%, 96%, 97%, 98%, or 99% sequence identity to the V_{H} region amino acid sequence set forth in any one of SEQ ID NOs: 1001, 1009, 1015, 1023, 1031, 1039, 1047, 1053, 1059, 1066, 1073, 1078, 1084, 1090, 1094, 1100, and has a V_{L} region having the amino acid sequence set forth in any one of SEQ ID NOs: 1005, 1013, 1019, 1027, 1035, 1043, 1051, 1056, 1063, 1070, 1076, 1082, 1088, 1093, 1097, 1103, 1125, 1154, 1155, 1156, 1178, 1233, 1234, or 1251, or an amino acid sequence that has at least at or about 85%, 86%, 87%, 88%, 89%, 90%, 91%, 92%, 93%, 94%, 95%, 96%, 97%, 98%, or 99% sequence identity to the V_{L} region amino acid sequence set forth in any one of SEQ ID NOs: 1005, 1013, 1019, 1027, 1035, 1043, 1051, 1056, 1063, 1070, 1076, 1082, 1088, 1093, 1097, 1103, 1125, 1154, 1155, 1156, 1178, 1233, 1234, or 1251.

In some embodiments, the antibody or antigen-binding fragment provided herein, the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1001, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1005; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1009, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1013; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1015, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1019 or 1125; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1023, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1027; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1031, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1035; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1039, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1043; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1047, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1051; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1053, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1056; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1059, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1063; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1066, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1070; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1073, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1076; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1078, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1082; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1084, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1088; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1090, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1093; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 10 94, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1097; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1100, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1103; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1031, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1154; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1039, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1155; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1059, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1156; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1066, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1233; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1094, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1234; the V_{H} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1096, and the V_{L} region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 1251.

In some embodiments, the V_{H} region of the antibody or antigen-binding fragment thereof comprises the amino acid sequence of any one of SEQ ID NOs: 1001, 1009, 1015, 1023, 1031, 1039, 1047, 1053, 1059, 1066, 1073, 1078, 1084, 1090, 1094, 1100, and the V_{L} region of the antibody or antigen-binding fragment comprises the amino acid sequence of any one of SEQ ID NOs: 1005, 1013, 1019, 1027, 1035, 1043, 1051, 1056, 1063, 1070, 1076, 1082, 1088, 1093, 1097, 1125, 1103, 1154, 1155, 1156, 1178, 1233, 1234, 1251.

In some embodiments of the antibody or antigen-binding fragment provided herein, the V_{H} region and the V_{L} region are or comprise the sequence set forth in SEQ ID NO: 1001 and 1005, respectively. In some embodiments of the antibody or antigen-binding fragment provided herein, the V_{H} region and the V_{L} region are or comprise the sequence set forth in SEQ ID NO: 1009 and 1013, respectively. In some embodiments of the antibody or antigen-binding fragment provided herein, the V_{H} region and the V_{L} region are or comprise the sequence set forth in SEQ ID NO: 1015 and 1019, respectively. In some embodiments of the antibody or antigen-binding fragment provided herein, the V_{H} region and the V_{L} region are or comprise the sequence set forth in SEQ ID NO: 1015 and 1125, respectively. In some embodiments of the antibody or antigen-binding fragment provided herein, the V_{H} region and the V_{L} region are or comprise the sequence set forth in SEQ ID NO: 1023 and 1027, respectively. In some embodiments of the antibody or antigen-binding fragment provided herein, the V_{H} region and the V_{L} region are or comprise the sequence set forth in SEQ ID NO: 1031 and 1035, respectively. In some embodiments of the antibody or antigen-binding fragment provided herein, the V_{H} region and the V_{L} region are or comprise the sequence set forth in SEQ ID NO: 1039 and 1043, respectively. In some embodiments of the antibody or antigen-binding fragment provided herein, the V_{H} region and the V_{L} region are or comprise the sequence set forth in SEQ ID NO: 1047 and 1051, respectively. In some embodiments of the antibody or antigen-binding fragment provided herein, the V_{H} region and the V_{L} region are or comprise the sequence set forth in SEQ ID NO: 1053 and 1056, respectively. In some embodiments of the antibody or antigen-binding fragment provided herein, the V_{H} region and the V_{L} region are or comprise the sequence set forth in SEQ ID NO: 1059 and 1063, respectively. In some embodiments of the antibody or antigen-binding fragment provided herein, the V_{H} region and the V_{L} region are or comprise the sequence set forth in SEQ ID NO: 1066 and 1070, respectively. In some embodiments of the antibody or antigen-binding fragment provided herein, the V_{H} region and the V_{L} region are or comprise the sequence set forth in SEQ ID NO: 1073 and 1076, respectively. In some embodiments of the antibody or antigen-binding fragment provided herein, the V_{H} region and the V_{L} region are or comprise the sequence set forth in SEQ ID NO: 1078 and 1082, respectively. In some embodiments of the antibody or antigen-binding fragment provided herein, the V_{H} region and the V_{L} region are or comprise the sequence set forth in SEQ ID NO: 1084 and 1088, respectively. In some embodiments of the antibody or antigen-binding fragment provided herein, the V_{H} region and the V_{L} region are or comprise the sequence set forth in SEQ ID NO: 1090 and 1093, respectively. In some embodiments of the antibody or antigen-binding fragment provided herein, the V_{H} region and the V_{L} region are or comprise the sequence set forth in SEQ ID NO: 1094 and 1097, respectively. In some embodiments of the antibody or antigen-binding fragment provided herein, the V_{H} region and the V_{L} region are or comprise the sequence set forth in SEQ ID NO: 1100 and 1103, respectively. In some embodiments of the antibody or antigen-binding fragment provided herein, the V_{H} region and the V_{L} region are or comprise the sequence set forth in SEQ ID NO: 1031 and 1154, respectively. In some embodiments of the antibody or antigen-binding fragment provided herein, the V_{H} region and the V_{L} region are or comprise the sequence set forth in SEQ ID NO: 1039 and 1155, respectively. In some embodiments of the antibody or antigen-binding fragment provided herein, the V_{H} region and the V_{L} region are or comprise the sequence set forth in SEQ ID NO: 1059 and 1156, respectively. In some embodiments of the antibody or antigen-binding fragment provided herein, the V_{H} region and the V_{L} region are or comprise the sequence set forth in SEQ ID NO: 1066 and 1233, respectively. In some embodiments of the antibody or antigen-binding fragment provided herein, the V_{H} region and the V_{L} region are or comprise the sequence set forth in SEQ ID NO: 1094 and 1234, respectively. In some embodiments of the antibody or antigen-binding fragment provided herein, the V_{H} region and the V_{L} region are or comprise the sequence set forth in SEQ ID NO: 1100 and 1250, respectively

Table 9 provides exemplary amino acid sequences of antibody heavy chain variable domains (VHs) and light chain variable domains (VLs) that, in combination, bind to SLC34A2, with CDR-H1, CDR-H2, CDR-H3, CDR-L1, CDR-L2, and CDR-L3 sequences noted below the respective V_{H} or V_{L} sequence. The CDR sequences provided in Table 9 are annotated using the Kabat scheme.

TABLE 9
SLC34A2 VH and VL Amino Acid Sequences

| Clone | VH Sequence | VL Sequence |
|---|---|---|
| SLC34A2 | QVQLQESGPGLVKPSETLSLTCTVSGGSVSSG | EIVLTQSPDFQSVTPKEKVTITCRASQSVGSG |
| Ab 1 | NFYWSWIRQPPGKGLEWIGYIYYSGSTYYNP | LHWYQQKPDQSPKLLIKYASQSFSGVPSRFSG |
| | SLKSRVTISVDTSKNQFSLKLRSLTAADTAV | SGSGTDFTLTINSLEVEDAATFYCLQSSSLPW |
| | YYCARWMTKVKGYFDYWGQGTLVTVSS | TFGQGTKVEIK (SEQ ID NO: 1005) |
| | (SEQ ID NO: 1001) | RASQSVGSGLH (SEQ ID NO: 1006) |
| | SGNFYWS (SEQ ID NO: 1002) | YASQSFS (SEQ ID NO: 1007) |
| | YIYYSGSTYYNPSLKS (SEQ ID NO: | LQSSSLPWT (SEQ ID NO: 1008) |
| | 1003) | |
| | WMTKVKGYFDY (SEQ ID NO: 1004) | |
| SLC34A2 | QVQLQESGPGLVKPSETLSLTCTVSGGSVSSG | EIVLTQSPDFQSVTPKEKVTITCRASQSVGSG |
| Ab 2 | SYYWSWIRQAPGKGLEWIGYIYYSGSNYYNP | LHWYQQKPDQSPKLLIKYASQSFSGVPSRFSG |
| | SLKSRVTISVDTSKNQFSLKLRAVTAADTAV | SGSGTDFTLTINSLETEDAATYFCQQSSSLPW |
| | YYCARWMTTIKGYFDYWGQGTLVTVSS | TFGQGTKVEIK (SEQ ID NO: 1013) |
| | (SEQ ID NO: 1009) | RASQSVGSGLH (SEQ ID NO: 1006) |
| | SGSYYWS (SEQ ID NO: 1010) | YASQSFS (SEQ ID NO: 1007) |
| | YIYYSGSNYYNPSLKS (SEQ ID NO: | QQSSSLPWT (SEQ ID NO: 1014) |
| | 1011) | |
| | WMTTIKGYFDY (SEQ ID NO: 1012) | |
| SLC34A2 | QVQLVESGGGLVQPGRSLRLSCTGSGFTFGDY | AIRMTQSPSSLSASVGDRVTITCRASQDINNY |
| Ab 3.1 | AMNWVRQAPGKGLEWVGFIRTKPYGGTTEYA | LAWYQQKPGKVPKLLIYAASTLQSGVPSRFSG |
| | ASVKGRFTFSRDDSKSIAYLQMNSLKTEDTA | SGSGTDFTLTISSLQPEDVATYYSLNYYSVPW |
| | VYYCTMIPVLRFLEWLPWGQGTLVTVSS | TFGQGTKVEIK (SEQ ID NO: 1019) |
| | (SEQ ID NO: 1015) | RASQDINNYLA (SEQ ID NO: 1020) |
| | DYAMN (SEQ ID NO: 1016) | AASTLQS (SEQ ID NO: 1021) |
| | FIRTKPYGGTTEYAASVKG (SEQ ID NO: | LNYYSVPWT (SEQ ID NO: 1022) |
| | 1017) | |
| | IPVLRFLEWLP (SEQ ID NO: 1018) | |
| SLC34A2 | QVQLVESGGGLVQPGRSLRLSCTGSGFTFGDY | DIQMTQSPSSLSASVGDRVTITCRASQDINNY |
| Ab 3 | AMNWVRQAPGKGLEWVGFIRTKPYGGTTEYA | LAWYQQKPGKVPKLLIYAASTLQSGVPSRFSG |
| | ASVKGRFTFSRDDSKSIAYLQMNSLKTEDTA | SGSGTDFTLTISSLQPEDVATYYSLNYYSVPW |
| | VYYCTMIPVLRFLEWLPWGQGTLVTVSS | TFGQGTKVEIK (SEQ ID NO: 1125) |
| | (SEQ ID NO: 1015) | RASQDINNYLA (SEQ ID NO: 1020) |
| | DYAMN (SEQ ID NO: 1016) | AASTLQS (SEQ ID NO: 1021) |
| | FIRTKPYGGTTEYAASVKG (SEQ ID NO: | LNYYSVPWT (SEQ ID NO: 1022) |
| | 1017) | |
| | IPVLRFLEWLP (SEQ ID NO: 1018) | |
| SLC34A2 | QVQLLESGGGLVQPGGSLRLSCAASGFTFSSY | EIVLTQSPSSLSASVGDRVTITCRASQGISNY |
| Ab 4 | AMNWVRQAPGKGLEWVSAISGGVGNTYYADS | LAWYQQKPGKVPNLLIYTASTLQSGVPSRFSG |
| | VKGRFTISRDNSKNTLYLQMNSLRAEDTAVY | SGSGTDFTLTISSLQPEDVATYYCQKYNSAPF |
| | YCTKDGPLWGNYFDYWGRGTLVTVSS (SEQ | TFGPGTKVDIK (SEQ ID NO: 1027) |
| | ID NO: 1023) | RASQGISNYLA (SEQ ID NO: 1028) |
| | SYAMN (SEQ ID NO: 1024) | TASTLQS (SEQ ID NO: 1029) |
| | AISGGVGNTYYADSVKG (SEQ ID NO: | QKYNSAPFT (SEQ ID NO: 1030) |
| | 1025) | |
| | DGPLWGNYFDY (SEQ ID NO: 1026) | |
| SLC34A2 | QVQLQESGPGLVKPSETLSLTCSVSGGSISSS | AIRMTQSPSSLSASVGDRVTITCRASQSISSY |
| Ab 5.1 | SYYWGWIRQPPGKGLEWIGSIDYSGSTYYNP | LNWYQQKPGKAPKLLIYAASSLLTGVPSRFTG |
| | SLKSRVTISIDTSKNQFSLRLSSVTAAEKAV | SGSGTDFTLTISSLQPEDFGTYYCQQSYSPPL |
| | YYCARHGRGTIGYFDYWGQGTLVTVSS | TFGGGTKVEIK (SEQ ID NO: 1035) |
| | (SEQ ID NO: 1031) | RASQSISSYLN (SEQ ID NO: 1036) |
| | SSSYYWG (SEQ ID NO: 1032) | AASSLLT (SEQ ID NO: 1037) |
| | SIDYSGSTYYNPSLKS (SEQ ID NO: | QQSYSPPLT (SEQ ID NO: 1038) |
| | 1033) | |
| | HGRGTIGYFDY (SEQ ID NO: 1034) | |
| SLC34A2 | QVQLQESGPGLVKPSETLSLTCSVSGGSISSS | DIQMTQSPSSLSASVGDRVTITCRASQSISSY |
| Ab 5 | SYYWGWIRQPPGKGLEWIGSIDYSGSTYYNP | LNWYQQKPGKAPKLLIYAASSLLTGVPSRFTG |
| | SLKSRVTISIDTSKNQFSLRLSSVTAAEKAV | SGSGTDFTLTISSLQPEDFGTYYCQQSYSPPL |
| | YYCARHGRGTIGYFDYWGQGTLVTVSS | TFGGGTKVEIK (SEQ ID NO: 1154) |
| | (SEQ ID NO: 1031) | RASQSISSYLN (SEQ ID NO: 1036) |
| | SSSYYWG (SEQ ID NO: 1032) | AASSLLT (SEQ ID NO: 1037) |
| | SIDYSGSTYYNPSLKS (SEQ ID NO: | QQSYSPPLT (SEQ ID NO: 1038) |
| | 1033) | |
| | HGRGTIGYFDY (SEQ ID NO: 1034) | |
| SLC34A2 | QVQLLESGGGLVQPGGSLRLSCAASGFTFSSS | AIRMTQSPSSLSASVGDRVTIACRASQVISNY |
| Ab 6.1 | AMAWVRQAPGKGLEWVSAISSSGDNTYYADS | LAWYQQKPGKVPKLLIYVASTLQSGVPSRFSG |
| | VKGRFTISRDTSKNTLSLQMSSLRAEDTAIY | SGSGTDFTLTISSLQPEDVATYYCQNYNSAPW |
| | YCAKQGTNWGLYFDYWGQGTLVTVSS (SEQ | TFGQGTKLEIK (SEQ ID NO: 1043) |
| | ID NO: 1039) | RASQVISNYLA (SEQ ID NO: 1044) |
| | SSAMA (SEQ ID NO: 1040) | VASTLQS (SEQ ID NO: 1045) |
| | AISSSGDNTYYADSVKG (SEQ ID NO: | QNYNSAPWT (SEQ ID NO: 1046) |
| | 1041) | |
| | QGTNWGLYFDY (SEQ ID NO: 1042) | |
| SLC34A2 | QVQLLESGGGLVQPGGSLRLSCAASGFTFSSS | DIQMTQSPSSLSASVGDRVTIACRASQVISNY |
| Ab 6 | AMAWVRQAPGKGLEWVSAISSSGDNTYYADS | LAWYQQKPGKVPKLLIYVASTLQSGVPSRFSG |
| | VKGRFTISRDTSKNTLSLQMSSLRAEDTAIY | SGSGTDFTLTISSLQPEDVATYYCQNYNSAPW |
| | YCAKQGTNWGLYFDYWGQGTLVTVSS (SEQ | TFGQGTKLEIK (SEQ ID NO: 1155) |
| | ID NO: 1039) | RASQVISNYLA (SEQ ID NO: 1044) |
| | SSAMA (SEQ ID NO: 1040) | VASTLQS (SEQ ID NO: 1045) |
| | AISSSGDNTYYADSVKG (SEQ ID NO: | QNYNSAPWT (SEQ ID NO: 1046) |
| | 1041) | |
| | QGTNWGLYFDY (SEQ ID NO: 1042) | |
| SLC34A2 | QVQLQESGPELVKPSETLSITCTVSGGSISSR | EIVLTQSPSSLSASVGDRVTITCRASQSISSY |
| Ab 7 | SYYWGWIRQPPGKGLEWIGSIYYGGSTYYNP | LNWYQQKPGRAPELLIYAASSLQSGVPSRFSG |
| | SLKSRVTISADTSKNQFSLKLNSVTAADTAV | SGSGTDFTLTISSLQPEDFATYYCQQSFSSLT |
| | FYCVRHPAGYSTRWSAFDIWGQGTMVTVSS | FGQGTRLEIK (SEQ ID NO: 1051) |
| | (SEQ ID NO: 1047) | RASQSISSYLN (SEQ ID NO: 1036) |
| | SRSYYWG (SEQ ID NO: 1048) | AASSLQS (SEQ ID NO: 15) |
| | SIYYGGSTYYNPSLKS (SEQ ID NO: | QQSFSSLT (SEQ ID NO: 1052) |
| | 1049) | |
| | HPAGYSTRWSAFDI (SEQ ID NO: | |
| | 1050) | |
| SLC34A2 | QVQLQESGPGLVKPSETLSLTCTVSGGSISSR | EIVLTQSPSSLSASVGDRVTITCRASQSISSY |
| Ab 8 | SYYWGWIRQPPGKGPEWIGSIYYSGSTFYNP | LNWYQLKPGKAPKLLIYAASSLHSGVPSRFSG |
| | SLKSRVTISEDTSKSQFSLKVTSVTAADTAV | SGSGTDFTLTISSLQPADFATYYCQQAYISLT |
| | YYCARHPAGYSSSWSAFDIWGQGTMVTVSS | FGQGTRLEIK (SEQ ID NO: 1056) |
| | (SEQ ID NO: 1053) | RASQSISSYLN (SEQ ID NO: 1036) |
| | SRSYYWG (SEQ ID NO: 1048) | AASSLHS (SEQ ID NO: 1057) |
| | SIYYSGSTFYNPSLKS (SEQ ID NO: | QQAYISLT (SEQ ID NO: 1058) |
| | 1054) | |
| | HPAGYSSSWSAFDI (SEQ ID NO: 1055) | |
| SLC34A2 | QVQLVESGGGLVQPGRSLRLSCSGSGFTSGDY | AIRMTQSPSSLSAFVGDRVTITCRASQDIGNY |
| Ab 9.1 | AVSWVRQAPGKGLEWVGFIRTKPYGETTEYA | LAWYQQTPEKVPKLLIYAASTLQSGVPSRFSG |
| | ASVKGRFTISRDDSKSIAYLQMNSLKAEDTA | SGSGTDFTLTISSLQPEDVATYYCQNYYSVPW |
| | VFYCTFIPVSRFLEWLPWGQGIPVTVSS | TFGQGTKVEIK (SEQ ID NO: 1063) |
| | (SEQ ID NO: 1059) | RASQDIGNYLA (SEQ ID NO: 1064) |
| | DYAVS (SEQ ID NO: 1060) | AASTLQS (SEQ ID NO: 1021) |
| | FIRTKPYGETTEYAASVKG (SEQ ID NO: | QNYYSVPWT (SEQ ID NO: 1065) |
| | 1061) | |
| | IPVSRFLEWLP (SEQ ID NO: 1062) | |
| SLC34A2 | QVQLVESGGGLVQPGRSLRLSCSGSGFTSGDY | DIQMTQSPSSLSAFVGDRVTITCRASQDIGNY |
| Ab 9 | AVSWVRQAPGKGLEWVGFIRTKPYGETTEYA | LAWYQQTPEKVPKLLIYAASTLQSGVPSRFSG |
| | ASVKGRFTISRDDSKSIAYLQMNSLKAEDTA | SGSGTDFTLTISSLQPEDVATYYCQNYYSVPW |
| | VFYCTFIPVSRFLEWLPWGQGIPVTVSS | TFGQGTKVEIK (SEQ ID NO: 1156) |
| | (SEQ ID NO: 1059) | RASQDIGNYLA (SEQ ID NO: 1064) |
| | DYAVS (SEQ ID NO: 1060) | AASTLQS (SEQ ID NO: 1021) |
| | FIRTKPYGETTEYAASVKG (SEQ ID NO: | QNYYSVPWT (SEQ ID NO: 1065) |
| | 1061) | |
| | IPVSRFLEWLP (SEQ ID NO: 1062) | |
| SLC34A2 | QVQLLESGGGLVQPGASLRLSCAASGFTFSTY | AIRMTQSPSSLSASVGDRVTITCRASQGISNY |
| Ab 10.1 | AMTWVRQAPGKGLEWVSGINGGGDTTYYADS | LAWYQQRPGKVPKLLIYAASTLRSGVPSRFSG |
| | VKGRFTISRDNSKNTLYLQMNSLRAEDTAVY | SGSGTDFTLTISSLQPEDVATYYCQKYNSAPL |
| | YCAVRGYTYGYFFDYWGQGTLVTVSS (SEQ | TFGGGTKVEIK (SEQ ID NO: 1070) |
| | ID NO: 1066) | RASQGISNYLA (SEQ ID NO: 1028) |
| | TYAMT (SEQ ID NO: 1067) | AASTLRS (SEQ ID NO: 1071) |
| | GINGGGDTTYYADSVKG (SEQ ID NO: | QKYNSAPLT (SEQ ID NO: 1072) |
| | 1068) | |
| | RGYTYGYFFDY (SEQ ID NO: 1069) | |
| SLC34A2 | QVQLLESGGGLVQPGASLRLSCAASGFTFSTY | DIQMTQSPSSLSASVGDRVTITCRASQGISNY |
| Ab 10 | AMTWVRQAPGKGLEWVSGINGGGDTTYYADS | LAWYQQRPGKVPKLLIYAASTLRSGVPSRFSG |
| | VKGRFTISRDNSKNTLYLQMNSLRAEDTAVY | SGSGTDFTLTISSLQPEDVATYYCQKYNSAPL |
| | YCAVRGYTYGYFFDYWGQGTLVTVSS (SEQ | TFGGGTKVEIK (SEQ ID NO: 1233) |
| | ID NO: 1066) | RASQGISNYLA (SEQ ID NO: 1028) |
| | TYAMT (SEQ ID NO: 1067) | AASTLRS (SEQ ID NO: 1071) |
| | GINGGGDTTYYADSVKG (SEQ ID NO: | QKYNSAPLT (SEQ ID NO: 1072) |
| | 1068) | |
| | RGYTYGYFFDY (SEQ ID NO: 1069) | |
| SLC34A2 | QVQLQESGPGLVKPSETLSLTCTVSGGSISSS | EIVLTQSPSSLSASVGDRLTITCRASQTISSY |
| Ab 11 | SYYWGWIRQPPGKGLEWIGSLYYSGSTYYNP | LNWYQQKPGKAPKVLIYAASSLQSGVPSRFSG |
| | SLKSRVTISVDTSKNQFSLKLNSVTAADTAV | SGSGTDFTLTISSLQPEDFATYYCQQSFIIPY |
| | YYCTRHPRGIAARWGNWFDPWGQGTLVTVSS | TFGQGTKLEIK (SEQ ID NO: 1076) |
| | (SEQ ID NO: 1073) | RASQTISSYLN (SEQ ID NO: 1251) |
| | SSSYYWG (SEQ ID NO: 1032) | AASSLQS (SEQ ID NO: 15) |
| | SLYYSGSTYYNPSLKS (SEQ ID NO: | QQSFIIPYT (SEQ ID NO: 1077) |
| | 1074) | |
| | HPRGIAARWGNWFDP (SEQ ID NO: | |
| | 1075) | |
| SLC34A2 | QVQLQESGPGLVKPSETLSLTCTVSGGSISSS | EIVLTQSPSSLSASVGDRVTITCRASQSISSY |
| Ab 12 | SYYRGWIRQPPGKGLEWIGSIYYSGSTYYNP | LNWYQQKPGKAPKLLIYAASSLQSGVPSRFSG |
| | SLKSRVTISVDTSKNQFSLKLSSVTAADTAV | SGSGTDFTLTISSLQPEDFATYYCQQSYSTPF |
| | YYCARHPRGSYGANFDYWGQGTLVTVSS | TFGPGTKVDIK (SEQ ID NO: 1082) |
| | (SEQ ID NO: 1078) | RASQSISSYLN (SEQ ID NO: 1036) |
| | SSSYYRG (SEQ ID NO: 1079) | AASSLQS (SEQ ID NO: 15) |
| | SIYYSGSTYYNPSLKS (SEQ ID NO: | QQSYSTPFT (SEQ ID NO: 1083) |
| | 1080) | |
| | HPRGSYGANFDY (SEQ ID NO: 1081) | |
| SLC34A2 | QVQLVESGGGLVQPGRSLRLSCIGSGFTFGEY | EIVLTQSPSSLSASVGDRVTVTCRANQDINNY |
| Ab 13 | AMSWVRQAPGKGLEWVGFIRTKPYGGTTEFA | LAWYQQTPGKVPKLLIYAASTLQSGVPSRFSG |
| | ASVKGRFTMSRDDSKSIAYLEMNSLKTEDTA | SGSGTDFTLTISSLQPEDVATYYCQNYYSVPW |
| | VYYCTLIPALRFLEWLPWGQGTLVTVSS | TFGQGTKLEIK (SEQ ID NO: 1088) |
| | (SEQ ID NO: 1084) | RANQDINNYLA (SEQ ID NO: 1089) |
| | EYAMS (SEQ ID NO: 1085) | AASTLQS (SEQ ID NO: 1021) |
| | FIRTKPYGGTTEFAASVKG (SEQ ID NO: | QNYYSVPWT (SEQ ID NO: 1065) |
| | 1086) | |
| | IPALRFLEWLP (SEQ ID NO: 1087) | |
| SLC34A2 | QVQLQESGPGLVKPSETLSLTCTVSGGSVSSG | EIVLTQSPDFQSVTPKESVTITCRASQSVGSG |
| Ab 14 | SYYWSWIRQPPGKGLEWIGYIFYSGSTYYNP | LHWYQQKPDQSPKLLIKYASQSFSGVPSRFSG |
| | SLKSRVTISVDTSKNQFSLKLTSVTAADTAV | SGSGTDFTLTINSLEAEDAATYYCLQSSSLPW |
| | YFCARWMTTVKGYFDYWGQGTLVTVSS | TFGQGTKVEIK (SEQ ID NO: 1093) |
| | (SEQ ID NO: 1090) | RASQSVGSGLH (SEQ ID NO: 1006) |
| | SGSYYWS (SEQ ID NO: 1010) | YASQSFS (SEQ ID NO: 1007) |
| | YIFYSGSTYYNPSLKS (SEQ ID NO: | LQSSSLPWT (SEQ ID NO: 1008) |
| | 1091) | |
| | WMTTVKGYFDY (SEQ ID NO: 1092) | |
| SLC34A2 | QVQLQESGPGLVKPSETLSLTCTVSGGSVSSA | AIRMTQSPSSLSASVGDRVTITCRASQSISSY |
| Ab 15.1 | SYYWSWIRQPPGKGLEYIGYIYYSGSTYYNP | LNWYQQKPGKAPKFLISPASSLQSGVPSRFSG |
| | SLKSRVTISIDTSKNQFSLNLRSVTAADTAV | SGSGTDFTLTISSLQPEDFATYYCQQSYSIPW |
| | YYCARYIVGRPGFNWFDPWGQGTLVTVSS | TFGQGTKVEIK (SEQ ID NO: 1097) |
| | (SEQ ID NO: 1094) | RASQSISSYLN (SEQ ID NO: 1036) |
| | SASYYWS (SEQ ID NO: 1095) | PASSLQS (SEQ ID NO: 1098) |
| | YIYYSGSTYYNPSLKS (SEQ ID NO: | QQSYSIPWT (SEQ ID NO: 1099) |
| | 1003) | |
| | YIVGRPGFNWFDP (SEQ ID NO: 1096) | |
| SLC34A2 | QVQLQESGPGLVKPSETLSLTCTVSGGSVSSA | DIQMTQSPSSLSASVGDRVTITCRASQSISSY |
| Ab 15 | SYYWSWIRQPPGKGLEYIGYIYYSGSTYYNP | LNWYQQKPGKAPKFLISPASSLQSGVPSRFSG |
| | SLKSRVTISIDTSKNQFSLNLRSVTAADTAV | SGSGTDFTLTISSLQPEDFATYYCQQSYSIPW |
| | YYCARYIVGRPGFNWFDPWGQGTLVTVSS | TFGQGTKVEIK (SEQ ID NO: 1234) |
| | (SEQ ID NO: 1094) | RASQSISSYLN (SEQ ID NO: 1036) |
| | SASYYWS (SEQ ID NO: 1095) | PASSLQS (SEQ ID NO: 1098) |
| | YIYYSGSTYYNPSLKS (SEQ ID NO: | QQSYSIPWT (SEQ ID NO: 1099) |
| | 1003) | |
| | YIVGRPGFNWFDP (SEQ ID NO: 1096) | |
| SLC34A2 | QVQLQESGPGLVKPSETLSLTCTVSGDSVSSV | AIRMTQSPSSLSASVGDRVTITCRASQTISSY |
| Ab 16.1 | SYYWSWIRQPPGKGLEWIGYIWYSGSTYYNP | LNWYQQKPGKAPKLLISPASNLQSGVPSRFSG |
| | SLKSRVTISIDTSKNQFSLKLRSVTAADTAV | SGSGTDFTLTISSLQPEDFATYYCQQSYIIPW |
| | YYCARYIVGRPGFNWFDPWGQGTLVTVSS | TFGQGTKVEIK (SEQ ID NO: 1103) |
| | (SEQ ID NO: 1100) | RASQTISSYLN (SEQ ID NO: 1251) |
| | SVSYYWS (SEQ ID NO: 1101) | PASNLQS (SEQ ID NO: 1104) |
| | YIWYSGSTYYNPSLKS (SEQ ID NO: | QQSYIIPWT (SEQ ID NO: 1105) |
| | 1102) | |
| | YIVGRPGFNWFDP (SEQ ID NO: 1096) | |
| SLC34A2 | QVQLQESGPGLVKPSETLSLTCTVSGDSVSSV | DIQMTQSPSSLSASVGDRVTITCRASQTISSY |
| Ab 16 | SYYWSWIRQPPGKGLEWIGYIWYSGSTYYNP | LNWYQQKPGKAPKLLISPASNLQSGVPSRFSG |
| | SLKSRVTISIDTSKNQFSLKLRSVTAADTAV | SGSGTDFTLTISSLQPEDFATYYCQQSYIIPW |
| | YYCARYIVGRPGFNWFDPWGQGTLVTVSS | TFGQGTKVEIK (SEQ ID NO: 1250) |
| | (SEQ ID NO: 1100) | RASQTISSYLN (SEQ ID NO: 1251) |
| | SVSYYWS (SEQ ID NO: 1101) | PASNLQS (SEQ ID NO: 1104) |
| | YIWYSGSTYYNPSLKS (SEQ ID NO: | QQSYIIPWT (SEQ ID NO: 1105) |
| | 1102) | |
| | YIVGRPGFNWFDP (SEQ ID NO: 1096) | |

In various embodiments, the antigen-binding fragment that binds to SLC34A2 comprises an scFv. In some embodiments, the scFv has the format V_{H}-L-V_{L} or V_{L}-L-V_{H}, wherein L is a linker peptide and the V_{H} and V_{L} are any V_{H} and V_{L} disclosed herein. In some embodiments, the scFv has the format V_{H}-L-V_{L}, wherein L is a linker peptide. In some embodiments, the scFv has the format V_{L}-L-V_{H}, wherein L is a linker peptide. In some embodiments, the linker peptide comprises the amino acid sequence of GGGGSGGGGSGGGGS (SEQ ID NO: 819). In some embodiments, the linker peptide comprises the amino acid sequence of GGGGSGSGGGGSGGGGS (SEQ ID NO: 820). In some embodiments, the linker peptide comprises the amino acid sequence of GSTSGSGKPGSGEGSTKG (SEQ ID NO: 998). Table 10 provides exemplary amino acid sequences of scFvs that bind to SLC34A2. The linker peptide linking the V_{H} to the V_{L} is indicated in bold italic text.

TABLE 10
SLC34A2 scFv Amino Acid Sequences

| Clone | scFv Sequence |
|---|---|
| SLC34A2 Ab 1 | EIVLTQSPDFQSVTPKEKVTITCRASQSVGSGLHWYQQKPDQSPKLLIKYASQSFSGVPSRF |
| scFv (VL-VH) | SGSGSGTDFTLTINSLEVEDAATFYCLQSSSLPWTFGQGTKVEIKGSTSGSGKPGSGEGSTK |
| SEQ ID NO: 1107 | GQVQLQESGPGLVKPSETLSLTCTVSGGSVSSGNFYWSWIRQPPGKGLEWIGYIYYSGSTYY |
| | NPSLKSRVTISVDTSKNQFSLKLRSLTAADTAVYYCARWMTKVKGYFDYWGQGTLVTVSS |
| SLC34A2 Ab 1 | QVQLQESGPGLVKPSETLSLTCTVSGGSVSSGNFYWSWIRQPPGKGLEWIGYIYYSGSTYYN |
| scFv (VH-VL) | PSLKSRVTISVDTSKNQFSLKLRSLTAADTAVYYCARWMTKVKGYFDYWGQGTLVTVSSGST |
| SEQ ID NO: 1126 | SGSGKPGSGEGSTKGEIVLTQSPDFQSVTPKEKVTITCRASQSVGSGLHWYQQKPDQSPKLL |
| | IKYASQSFSGVPSRFSGSGSGTDFTLTINSLEVEDAATFYCLQSSSLPWTFGQGTKVEIK |
| SLC34A2 Ab 2 | EIVLTQSPDFQSVTPKEKVTITCRASQSVGSGLHWYQQKPDQSPKLLIKYASQSFSGVPSRF |
| scFv | SGSGSGTDFTLTINSLETEDAATYFCQQSSSLPWTFGQGTKVEIKGSTSGSGKPGSGEGSTK |
| (VL-VH) | GQVQLQESGPGLVKPSETLSLTCTVSGGSVSSGSYYWSWIRQAPGKGLEWIGYIYYSGSNYY |
| SEQ ID NO: 1108 | NPSLKSRVTISVDTSKNQFSLKLRAVTAADTAVYYCARWMTTIKGYFDYWGQGTLVTVSS |
| SLC34A2 Ab 2 | QVQLQESGPGLVKPSETLSLTCTVSGGSVSSGSYYWSWIRQAPGKGLEWIGYIYYSGSNYYN |
| scFv (VH-VL) | PSLKSRVTISVDTSKNQFSLKLRAVTAADTAVYYCARWMTTIKGYFDYWGQGTLVTVSSGST |
| SEQ ID NO: 1127 | SGSGKPGSGEGSTKGEIVLTQSPDFQSVTPKEKVTITCRASQSVGSGLHWYQQKPDQSPKLL |
| | IKYASQSFSGVPSRFSGSGSGTDFTLTINSLETEDAATYFCQQSSSLPWTFGQGTKVEIK |
| SLC34A2 Ab 3 | DIQMTQSPSSLSASVGDRVTITCRASQDINNYLAWYQQKPGKVPKLLIYAASTLQSGVPSRF |
| scFv (VL-VH) | SGSGSGTDFTLTISSLQPEDVATYYSLNYYSVPWTFGQGTKVEIKGSTSGSGKPGSGEGSTK |
| SEQ ID NO: 1109 | GQVQLVESGGGLVQPGRSLRLSCTGSGFTFGDYAMNWVRQAPGKGLEWVGFIRTKPYGGTTE |
| | YAASVKGRFTFSRDDSKSIAYLQMNSLKTEDTAVYYCTMIPVLRFLEWLPWGQGTLVTVSS |
| SLC34A2 Ab 3 | QVQLVESGGGLVQPGRSLRLSCTGSGFTFGDYAMNWVRQAPGKGLEWVGFIRTKPYGGTTEY |
| scFv (VH-VL) | AASVKGRFTFSRDDSKSIAYLQMNSLKTEDTAVYYCTMIPVLRFLEWLPWGQGTLVTVSSGS |
| SEQ ID NO: 1128 | TSGSGKPGSGEGSTKGDIQMTQSPSSLSASVGDRVTITCRASQDINNYLAWYQQKPGKVPKL |
| | LIYAASTLQSGVPSRFSGSGSGTDFTLTISSLQPEDVATYYSLNYYSVPWTFGQGTKVEIK |
| SLC34A2 Ab 4 | DIQMTQSPSSLSASVGDRVTITCRASQGISNYLAWYQQKPGKVPNLLIYTASTLQSGVPSRF |
| scFv (VL-VH) | SGSGSGTDFTLTISSLQPEDVATYYCQKYNSAPFTFGPGTKVDIKGSTSGSGKPGSGEGSTK |
| SEQ ID NO: | GQVQLLESGGGLVQPGGSLRLSCAASGFTFSSYAMNWVRQAPGKGLEWVSAISGGVGNTYYA |
| 1129 | DSVKGRFTISRDNSKNTLYLQMNSLRAEDTAVYYCTKDGPLWGNYFDYWGRGTLVTVSS |
| SLC34A2 Ab 4 | QVQLLESGGGLVQPGGSLRLSCAASGFTFSSYAMNWVRQAPGKGLEWVSAISGGVGNTYYAD |
| scFv (VH-VL) | SVKGRFTISRDNSKNTLYLQMNSLRAEDTAVYYCTKDGPLWGNYFDYWGRGTLVTVSSGSTS |
| SEQ ID NO: 1130 | GSGKPGSGEGSTKGDIQMTQSPSSLSASVGDRVTITCRASQGISNYLAWYQQKPGKVPNLLI |
| | YTASTLQSGVPSRFSGSGSGTDFTLTISSLQPEDVATYYCQKYNSAPFTFGPGTKVDIK |
| SLC34A2 Ab 5 | DIQMTQSPSSLSASVGDRVTITCRASQSISSYLNWYQQKPGKAPKLLIYAASSLLTGVPSRF |
| scFv (VL-VH) | TGSGSGTDFTLTISSLQPEDFGTYYCQQSYSPPLTFGGGTKVEIKGSTSGSGKPGSGEGSTK |
| SEQ ID NO: 1131 | GQVQLQESGPGLVKPSETLSLTCSVSGGSISSSSYYWGWIRQPPGKGLEWIGSIDYSGSTYY |
| | NPSLKSRVTISIDTSKNQFSLRLSSVTAAEKAVYYCARHGRGTIGYFDYWGQGTLVTVSS |
| SLC34A2 Ab 6 | DIQMTQSPSSLSASVGDRVTIACRASQVISNYLAWYQQKPGKVPKLLIYVASTLQSGVPSRF |
| scFv (VL-VH) | SGSGSGTDFTLTISSLQPEDVATYYCQNYNSAPWTFGQGTKLEIKGSTSGSGKPGSGEGSTK |
| SEQ ID NO: 1132 | GQVQLLESGGGLVQPGGSLRLSCAASGFTFSSSAMAWVRQAPGKGLEWVSAISSSGDNTYYA |
| | DSVKGRFTISRDTSKNTLSLQMSSLRAEDTAIYYCAKQGTNWGLYFDYWGQGTLVTVSS |
| SLC34A2 Ab 6 | QVQLLESGGGLVQPGGSLRLSCAASGFTESSSAMAWVRQAPGKGLEWVSAISSSGDNTYYAD |
| scFv (VH-VL) | SVKGRFTISRDTSKNTLSLQMSSLRAEDTAIYYCAKQGTNWGLYFDYWGQGTLVTVSSGSTS |
| SEQ ID NO: 1133 | GSGKPGSGEGSTKGDIQMTQSPSSLSASVGDRVTIACRASQVISNYLAWYQQKPGKVPKLLI |
| | YVASTLQSGVPSRFSGSGSGTDFTLTISSLQPEDVATYYCQNYNSAPWTFGQGTKLEIK |
| SLC34A2 Ab 7 | EIVLTQSPSSLSASVGDRVTITCRASQSISSYLNWYQQKPGRAPELLIYAASSLQSGVPSRF |
| scFv (VL-VH) | SGSGSGTDFTLTISSLQPEDFATYYCQQSFSSLTFGQGTRLEIKGSTSGSGKPGSGEGSTKG |
| SEQ ID NO: 1134 | QVQLQESGPELVKPSETLSITCTVSGGSISSRSYYWGWIRQPPGKGLEWIGSIYYGGSTYYN |
| | PSLKSRVTISADTSKNQFSLKLNSVTAADTAVFYCVRHPAGYSTRWSAFDIWGQGTMVTVSS |
| SLC34A2 Ab 8 | EIVLTQSPSSLSASVGDRVTITCRASQSISSYLNWYQLKPGKAPKLLIYAASSLHSGVPSRF |
| scFv (VL-VH) | SGSGSGTDFTLTISSLQPADFATYYCQQAYISLTFGQGTRLEIKGSTSGSGKPGSGEGSTKG |
| SEQ ID NO: 1135 | QVQLQESGPGLVKPSETLSLTCTVSGGSISSRSYYWGWIRQPPGKGPEWIGSIYYSGSTFYN |
| | PSLKSRVTISEDTSKSQFSLKVTSVTAADTAVYYCARHPAGYSSSWSAFDIWGQGTMVTVSS |
| SLC34A2 Ab 9 | DIQMTQSPSSLSAFVGDRVTITCRASQDIGNYLAWYQQTPEKVPKLLIYAASTLQSGVPSRF |
| scFv (VL-VH) | SGSGSGTDFTLTISSLQPEDVATYYCQNYYSVPWTFGQGTKVEIKGSTSGSGKPGSGEGSTK |
| SEQ ID NO: 1136 | GQVQLVESGGGLVQPGRSLRLSCSGSGFTSGDYAVSWVRQAPGKGLEWVGFIRTKPYGETTE |
| | YAASVKGRFTISRDDSKSIAYLQMNSLKAEDTAVFYCTFIPVSRFLEWLPWGQGIPVTVSS |
| SLC34A2 Ab 10 | DIQMTQSPSSLSASVGDRVTITCRASQGISNYLAWYQQRPGKVPKLLIYAASTLRSGVPSRF |
| scFv (VL-VH) | SGSGSGTDFTLTISSLQPEDVATYYCQKYNSAPLTFGGGTKVEIKGSTSGSGKPGSGEGSTK |
| SEQ ID NO: 1137 | GQVQLLESGGGLVQPGASLRLSCAASGFTFSTYAMTWVRQAPGKGLEWVSGINGGGDTTYYA |
| | DSVKGRFTISRDNSKNTLYLQMNSLRAEDTAVYYCAVRGYTYGYFFDYWGQGTLVTVSS |
| SLC34A2 Ab 11 | EIVLTQSPSSLSASVGDRLTITCRASQTISSYLNWYQQKPGKAPKVLIYAASSLQSGVPSRF |
| scFv (VL-VH) | SGSGSGTDFTLTISSLQPEDFATYYCQQSFIIPYTFGQGTKLEIKGSTSGSGKPGSGEGSTK |
| SEQ ID NO: 1138 | GQVQLQESGPGLVKPSETLSLTCTVSGGSISSSSYYWGWIRQPPGKGLEWIGSLYYSGSTYY |
| | NPSLKSRVTISVDTSKNQFSLKLNSVTAADTAVYYCTRHPRGIAARWGNWEDPWGQGTLVTV |
| | SS |
| SLC34A2 Ab 12 | EIVLTQSPSSLSASVGDRVTITCRASQSISSYLNWYQQKPGKAPKLLIYAASSLQSGVPSRF |
| scFv (VL-VH) | SGSGSGTDFTLTISSLQPEDFATYYCQQSYSTPFTFGPGTKVDIKGSTSGSGKPGSGEGSTK |
| SEQ ID NO: 1139 | GQVQLQESGPGLVKPSETLSLTCTVSGGSISSSSYYRGWIRQPPGKGLEWIGSIYYSGSTYY |
| | NPSLKSRVTISVDTSKNQFSLKLSSVTAADTAVYYCARHPRGSYGANFDYWGQGTLVTVSS |
| SLC34A2 Ab 12 | QVQLQESGPGLVKPSETLSLTCTVSGGSISSSSYYRGWIRQPPGKGLEWIGSIYYSGSTYYN |
| scFv (VH-VL) | PSLKSRVTISVDTSKNQFSLKLSSVTAADTAVYYCARHPRGSYGANFDYWGQGTLVTVSSGS |
| SEQ ID NO: 1140 | TSGSGKPGSGEGSTKGEIVLTQSPSSLSASVGDRVTITCRASQSISSYLNWYQQKPGKAPKL |
| | LIYAASSLQSGVPSRFSGSGSGTDFTLTISSLQPEDFATYYCQQSYSTPFTFGPGTKVDIK |
| SLC34A2 Ab 13 | DIQMTQSPSSLSASVGDRVTVTCRANQDINNYLAWYQQTPGKVPKLLIYAASTLQSGVPSRF |
| scFv (VL-VH) | SGSGSGTDFTLTISSLQPEDVATYYCQNYYSVPWTFGQGTKLEIKGSTSGSGKPGSGEGSTK |
| SEQ ID NO: 1141 | GQVQLVESGGGLVQPGRSLRLSCIGSGFTFGEYAMSWVRQAPGKGLEWVGFIRTKPYGGTTE |
| | FAASVKGRFTMSRDDSKSIAYLEMNSLKTEDTAVYYCTLIPALRFLEWLPWGQGTLVTVSS |
| SLC34A2 Ab 14 | EIVLTQSPDFQSVTPKESVTITCRASQSVGSGLHWYQQKPDQSPKLLIKYASQSFSGVPSRF |
| scFv (VL-VH) | SGSGSGTDFTLTINSLEAEDAATYYCLQSSSLPWTFGQGTKVEIKGSTSGSGKPGSGEGSTK |
| SEQ ID NO: 1142 | GQVQLQESGPGLVKPSETLSLTCTVSGGSVSSGSYYWSWIRQPPGKGLEWIGYIFYSGSTYY |
| | NPSLKSRVTISVDTSKNQFSLKLTSVTAADTAVYFCARWMTTVKGYFDYWGQGTLVTVSS |
| SLC34A2 Ab 15 | DIQMTQSPSSLSASVGDRVTITCRASQSISSYLNWYQQKPGKAPKFLISPASSLQSGVPSRF |
| scFv (VL-VH) | SGSGSGTDFTLTISSLQPEDFATYYCQQSYSIPWTFGQGTKVEIKGSTSGSGKPGSGEGSTK |
| SEQ ID NO: 1143 | GQVQLQESGPGLVKPSETLSLTCTVSGGSVSSASYYWSWIRQPPGKGLEYIGYIYYSGSTYY |
| | NPSLKSRVTISIDTSKNQFSLNLRSVTAADTAVYYCARYIVGRPGFNWFDPWGQGTLVTVSS |
| SLC34A2 Ab 16 | DIQMTQSPSSLSASVGDRVTITCRASQTISSYLNWYQQKPGKAPKLLISPASNLQSGVPSRF |
| scFv (VL-VH) | SGSGSGTDFTLTISSLQPEDFATYYCQQSYIIPWTFGQGTKVEIKGSTSGSGKPGSGEGSTK |
| SEQ ID NO: 1144 | GQVQLQESGPGLVKPSETLSLTCTVSGDSVSSVSYYWSWIRQPPGKGLEWIGYIWYSGSTYY |
| | NPSLKSRVTISIDTSKNQFSLKLRSVTAADTAVYYCARYIVGRPGENWEDPWGQGTLVTVSS |
| SLC34A2 Ab 16 | QVQLQESGPGLVKPSETLSLTCTVSGDSVSSVSYYWSWIRQPPGKGLEWIGYIWYSGSTYYN |
| scFv (VH-VL) | PSLKSRVTISIDTSKNQFSLKLRSVTAADTAVYYCARYIVGRPGFNWEDPWGQGTLVTVSSG |
| SEQ ID NO: 1145 | STSGSGKPGSGEGSTKGDIQMTQSPSSLSASVGDRVTITCRASQTISSYLNWYQQKPGKAPK |
| | LLISPASNLQSGVPSRFSGSGSGTDFTLTISSLQPEDFATYYCQQSYIIPWTFGQGTKVEIK |

In some embodiments, the antibody or antigen-binding fragment that binds to SLC34A2 comprises an scFv comprising an amino acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to an amino acid sequence selected from the sequence as set forth in SEQ ID NOs: 1107, 1126, 1108, 1127, 1109, 1128, 1129, 1130, 1131, 1132, 1133, 1134, 1135, 1136, 1137, 1138, 1139, 1140, 1141, 1142, 1143, 1144, or 1145, optionally, wherein the V_{H} CDRs and the V_{L} CDRs are identical to those in SEQ ID NOs: 1107, 1126, 1108, 1127, 1109, 1128, 1129, 1130, 1131, 1132, 1133, 1134, 1135, 1136, 1137, 1138, 1139, 1140, 1141, 1142, 1143, 1144, or 1145, respectively. In some embodiments, the antibody or antigen-binding fragment that binds to SLC34A2 comprises an scFv comprising the amino acid sequence selected from the sequence as set forth in SEQ ID NOs: 1107, 1126, 1108, 1127, 1109, 1128, 1129, 1130, 1131, 1132, 1133, 1134, 1135, 1136, 1137, 1138, 1139, 1140, 1141, 1142, 1143, 1144, or 1145.

Table 11 provides exemplary nucleic acid sequences encoding V_{H} and V_{L} domains that, in combination, bind to SLC34A2. In some embodiments, the V_{H} and the V_{L} of the antibody or antigen-binding fragment that binds to SLC34A2 are each encoded by a sequence set forth in Table 11.

TABLE 11
SLC34A2 VH and VL Nucleic Acid Sequences

| Clone | VH Sequence | VL Sequence |
|---|---|---|
| SLC34A2 | CAAGTGCAACTGCAAGAGTCTGGACCCGGGC | GAAATCGTCCTGACACAGTCTCCAGATTTTCA |
| Ab 1 | TGGTGAAACCAAGCGAGACATTATCCCTCAC | GAGCGTGACGCCAAAGGAGAAAGTGACAATTA |
| | TTGTACCGTGTCAGGCGGTAGTGTGTCCTCC | CATGCCGGGCATCTCAGTCTGTTGGGTCTGGG |
| | GGGAATTTCTACTGGAGTTGGATACGCCAGC | TTGCATTGGTATCAGCAAAAGCCCGACCAGTC |
| | CTCCTGGGAAGGGCCTTGAATGGATTGGCTA | ACCCAAACTGCTCATCAAATATGCAAGCCAGA |
| | CATCTACTATTCAGGCTCCACCTACTACAAC | GTTTTTCAGGCGTACCTTCACGATTTAGCGGA |
| | CCGTCTTTGAAGTCAAGGGTTACGATAAGCG | AGTGGTTCTGGCACTGACTTCACCTTGACGAT |
| | TCGATACCTCCAAAAACCAATTCTCCCTAAA | TAATAGCCTGGAAGTAGAAGACGCTGCCACTT |
| | GCTCAGATCGTTAACTGCCGCTGATACCGCG | TCTACTGCCTGCAAAGTAGCTCCCTGCCCTGG |
| | GTGTACTATTGTGCCCGTTGGATGACCAAAG | ACTTTTGGGCAGGGTACTAAGGTCGAGATCAA |
| | TTAAGGGTTATTTCGACTATTGGGGACAAGG | G (SEQ ID NO: 1111) |
| | GACACTTGTCACCGTGTCCTCC (SEQ ID | |
| | NO: 1110) | |
| SLC34A2 | CAAGTGCAACTGCAAGAATCGGGACCCGGGC | GAGATAGTCCTGACCCAGTCACCCGATTTCCA |
| Ab 2 | TGGTTAAACCAAGTGAAACCCTTTCCCTCAC | GAGTGTTACTCCTAAGGAGAAAGTGACTATAA |
| | TTGTACCGTAAGCGGCGGTAGCGTGTCCTCT | CATGCCGGGCATCTCAGTCTGTCGGAAGCGGG |
| | GGTAGCTACTATTGGAGTTGGATTAGGCAGG | CTACATTGGTACCAGCAGAAGCCTGACCAGAG |
| | CGCCAGGGAAAGGCCTCGAATGGATTGGGTA | CCCGAAACTGCTCATCAAATATGCCTCCCAGT |
| | TATCTACTACAGCGGCAGTAACTACTACAAC | CGTTTTCTGGCGTGCCCTCTCGCTTTTCCGGA |
| | CCATCATTGAAGTCTAGAGTGACAATTAGTG | AGCGGATCTGGCACAGACTTCACCTTGACCAT |
| | TCGATACCTCTAAAAATCAATTCTCACTTAA | CAATAGCCTGGAAACTGAGGACGCCGCTACGT |
| | GCTGCGAGCTGTAACCGCCGCAGACACTGCG | ATTTCTGCCAGCAGTCCTCCAGTCTGCCTTGG |
| | GTGTACTATTGTGCCCGTTGGATGACCACTA | ACATTTGGTCAGGGAACGAAGGTGGAGATCAA |
| | TCAAGGGCTACTTCGATTATTGGGGACAAGG | G (SEQ ID NO: 1113) |
| | GACATTAGTTACGGTGTCCTCC (SEQ ID | |
| | NO: 1112) | |
| SLC34A2 | CAGGTCCAGCTCGTGGAGTCTGGCGGTGGGC | GATATTCAGATGACACAGAGCCCAAGTTCACT |
| Ab 3 | TGGTTCAGCCAGGGAGGAGTTTGCGGCTTTC | GTCAGCGAGTGTCGGAGATCGGGTTACTATAA |
| | CTGTACGGGAAGCGGATTCACCTTTGGTGAC | CCTGCCGAGCATCTCAGGATATCAATAATTAC |
| | TATGCCATGAACTGGGTCAGGCAGGCTCCTG | CTGGCCTGGTATCAGCAAAAACCCGGCAAAGT |
| | GAAAAGGCCTAGAGTGGGTGGGTTTCATCAG | CCCGAAGCTCCTGATATACGCAGCTTCAACAC |
| | AACCAAGCCATATGGCGGCACTACAGAATAT | TGCAAAGCGGTGTGCCCTCGCGCTTTAGCGGC |
| | GCAGCCAGCGTAAAAGGGAGATTCACGTTCA | TCTGGCAGCGGGACAGATTTCACCCTGACGAT |
| | GCCGCGACGACTCTAAGTCAATAGCTTACCT | TTCCTCCCTTCAACCCGAGGACGTGGCCACTT |
| | TCAGATGAACTCCCTCAAGACCGAAGACACC | ACTACTCCCTCAACTATTACTCTGTACCCTGG |
| | GCCGTGTACTATTGTACTATGATCCCTGTGC | ACCTTTGGCCAAGGGACAAAGGTGGAAATCAA |
| | TGCGTTTTTTAGAGTGGTTGCCTTGGGGACA | G (SEQ ID NO: 1115) |
| | AGGGACATTAGTTACTGTGTCCTCC (SEQ | |
| | ID NO: 1114) | |
| SLC34A2 | CAGGTCCAGCTCGTGGAGTCTGGCGGTGGGC | GCCATCCGGATGACACAGAGCCCAAGTTCACT |
| Ab 3.1 | TGGTTCAGCCAGGGAGGAGTTTGCGGCTTTC | GTCAGCGAGTGTCGGAGATCGGGTTACTATAA |
| | CTGTACGGGAAGCGGATTCACCTTTGGTGAC | CCTGCCGAGCATCTCAGGATATCAATAATTAC |
| | TATGCCATGAACTGGGTCAGGCAGGCTCCTG | CTGGCCTGGTATCAGCAAAAACCCGGCAAAGT |
| | GAAAAGGCCTAGAGTGGGTGGGTTTCATCAG | CCCGAAGCTCCTGATATACGCAGCTTCAACAC |
| | AACCAAGCCATATGGCGGCACTACAGAATAT | TGCAAAGCGGTGTGCCCTCGCGCTTTAGCGGC |
| | GCAGCCAGCGTAAAAGGGAGATTCACGTTCA | TCTGGCAGCGGGACAGATTTCACCCTGACGAT |
| | GCCGCGACGACTCTAAGTCAATAGCTTACCT | TTCCTCCCTTCAACCCGAGGACGTGGCCACTT |
| | TCAGATGAACTCCCTCAAGACCGAAGACACC | ACTACTCCCTCAACTATTACTCTGTACCCTGG |
| | GCCGTGTACTATTGTACTATGATCCCTGTGC | ACCTTTGGCCAAGGGACAAAGGTGGAAATCAA |
| | TGCGTTTTTTAGAGTGGTTGCCTTGGGGACA | G (SEQ ID NO: 999) |
| | AGGGACATTAGTTACTGTGTCCTCC (SEQ | |
| | ID NO: 1114) | |
| SLC34A2 | CAAGTGCAGCTGCTGGAATCTGGCGGCGGGC | GACATTCAGATGACTCAGTCACCTAGCTCACT |
| Ab 4 | TGGTGCAACCTGGAGGGAGCTTGCGGTTATC | GTCAGCGTCAGTCGGAGATCGGGTTACTATTA |
| | ATGCGCCGCCTCTGGCTTCACCTTCTCCTCC | CGTGTAGGGCTAGTCAGGGTATCAGCAATTAC |
| | TATGCCATGAATTGGGTTAGGCAGGCTCCTG | CTCGCCTGGTACCAGCAGAAACCCGGCAAAGT |
| | GGAAAGGTCTGGAGTGGGTAAGCGCTATTAG | CCCAAACCTGCTCATCTACACTGCAAGCACCC |
| | TGGTGGAGTGGGAAACACATATTATGCCGAT | TACAGAGCGGTGTTCCATCCCGTTTTTCTGGA |
| | TCTGTGAAGGGACGCTTTACAATCTCGAGAG | TCGGGATCTGGGACGGATTTCACCCTCACCAT |
| | ACAACAGCAAAAATACTCTGTACCTTCAAAT | ATCCTCCTTACAACCCGAGGACGTCGCAACTT |
| | GAACTCCCTCCGAGCCGAGGATACCGCAGTG | ATTACTGTCAGAAGTACAATAGTGCTCCCTTC |
| | TACTATTGCACAAAGGACGGGCCACTTTGGG | ACGTTTGGCCCGGGCACAAAGGTGGACATCAA |
| | GCAACTATTTTGATTACTGGGGCAGAGGAAC | G (SEQ ID NO: 1179) |
| | ATTGGTAACCGTGTCCTCC (SEQ ID | |
| | NO: 1178) | |
| SLC34A2 | CAGGTCCAACTACAGGAGTCGGGCCCTGGGC | GATATTCAAATGACTCAGAGTCCTTCTTCACT |
| Ab 5 | TGGTGAAGCCATCTGAAACCCTGTCACTCAC | GTCAGCCTCTGTTGGCGACCGGGTCACTATAA |
| | TTGCTCTGTTAGTGGTGGTTCAATCTCCTCC | CTTGTAGGGCTTCGCAGTCAATCTCCTCATAC |
| | AGCTCATATTACTGGGGCTGGATTAGACAGC | CTGAACTGGTACCAGCAGAAGCCCGGCAAAGC |
| | CACCCGGAAAAGGGTTAGAGTGGATAGGCAG | GCCGAAACTTTTGATATATGCAGCCAGTTCCT |
| | TATCGACTACAGCGGTAGCACCTATTACAAC | TACTGACCGGAGTACCTAGTCGCTTCACCGGC |
| | CCAAGCCTCAAGTCCCGAGTCACCATCAGTA | AGCGGGAGCGGGACAGACTTCACGCTCACGAT |
| | TCGATACAAGCAAGAATCAGTTCAGCCTGAG | TAGTTCCTTGCAGCCCGAGGACTTTGGAACCT |
| | GCTGTCCTCCGTGACAGCCGCTGAGAAAGCC | ATTACTGCCAACAGTCTTATAGCCCACCTCTT |
| | GTGTACTATTGTGCAAGACATGGCCGTGGGA | ACGTTTGGCGGCGGCACAAAGGTGGAAATCAA |
| | CTATTGGATATTTTGATTACTGGGGACAAGG | G (SEQ ID NO: 1181) |
| | GACACTCGTGACCGTGTCCTCC (SEQ ID | |
| | NO: 1180) | |
| SLC34A2 | CAGGTCCAGCTTCTGGAAAGCGGCGGCGGGC | GACATTCAGATGACCCAGTCTCCTAGTTCACT |
| Ab 6 | TGGTACAGCCAGGAGGCTCCTTGCGGTTATC | GTCAGCCTCTGTGGGCGACAGAGTGACCATTG |
| | ATGTGCCGCTAGTGGATTCACCTTTTCTAGC | CATGCAGGGCCTCTCAGGTTATTAGTAACTAC |
| | TCTGCAATGGCCTGGGTGCGCCAGGCACCGG | TTGGCTTGGTACCAGCAGAAACCTGGGAAGGT |
| | GAAAAGGTCTGGAGTGGGTCAGCGCAATATC | TCCCAAACTGCTGATATACGTAGCTTCGACTC |
| | AAGCTCTGGTGACAACACCTACTATGCCGAT | TACAGTCAGGCGTGCCATCCCGTTTTAGCGGA |
| | AGTGTCAAGGGAAGGTTCACCATCTCGAGAG | AGCGGTAGCGGCACTGATTTCACGCTCACCAT |
| | ACACATCTAAAAATACTCTTAGCCTTCAGAT | CTCCTCCCTGCAACCCGAGGATGTTGCCACGT |
| | GTCCTCCCTCCGAGCTGAGGACACAGCGATC | ATTATTGTCAAAACTACAATAGTGCGCCTTGG |
| | TACTATTGCGCTAAGCAAGGAACAAATTGGG | ACTTTCGGGCAAGGGACAAAGTTAGAGATCAA |
| | GACTCTACTTTGATTATTGGGGTCAGGGAAC | G (SEQ ID NO: 1183) |
| | ACTCGTGACTGTGTCCTCC (SEQ ID NO: | |
| | 1182) | |
| SLC34A2 | CAGGTCCAACTGCAAGAATCGGGCCCTGAGT | GAGATTGTCTTGACCCAGTCTCCCTCTTCACT |
| Ab 7 | TAGTTAAACCTAGCGAAACGTTGTCCATTAC | GTCGGCCTCTGTTGGGGACCGCGTAACCATTA |
| | ATGTACCGTGAGTGGTGGCTCTATCTCCTCC | CCTGCCGGGCCTCTCAGTCAATCAGTTCTTAC |
| | AGAAGTTATTATTGGGGCTGGATTAGACAGC | CTGAATTGGTACCAGCAGAAGCCAGGGCGGGC |
| | CACCTGGGAAAGGGCTCGAGTGGATCGGGTC | ACCCGAACTGCTGATCTACGCAGCCAGCAGCC |
| | CATATACTACGGAGGCAGCACCTACTATAAT | TGCAGTCTGGGGTGCCCTCGAGGTTTAGCGGC |
| | CCAAGTCTGAAGAGTCGAGTAACTATATCAG | TCAGGAAGCGGAACAGACTTCACACTCACAAT |
| | CGGATACGAGCAAGAACCAGTTTAGCCTTAA | CTCCTCCCTCCAACCCGAGGACTTCGCCACAT |
| | ACTTAACAGTGTGACCGCCGCTGATACTGCT | ACTATTGCCAGCAATCATTTTCCTCCCTAACC |
| | GTGTTCTATTGTGTCCGCCACCCGGCTGGAT | TTCGGCCAGGGTACTAGGCTCGAAATCAAG |
| | ATAGCACTCGTTGGAGTGCATTTGATATTTG | (SEQ ID NO: 1185) |
| | GGGACAAGGCACAATGGTGACTGTGTCCTCC | |
| | (SEQ ID NO: 1184) | |
| SLC34A2 | CAAGTGCAGCTCCAGGAGAGCGGGCCAGGGC | GAGATTGTGCTGACACAGAGCCCTTCTTCGCT |
| Ab 8 | TGGTGAAACCAAGTGAAACGCTGTCCCTGAC | GAGTGCCAGTGTCGGAGATCGGGTCACAATTA |
| | TTGCACGGTTAGCGGCGGCTCGATCTCCTCC | CCTGTAGGGCATCACAGTCAATCTCCTCCTAC |
| | AGATCATATTACTGGGGATGGATCCGACAGC | CTGAACTGGTACCAGTTGAAGCCTGGCAAGGC |
| | CTCCCGGCAAAGGGCCGGAGTGGATTGGAAG | TCCCAAACTTTTGATATACGCTGCTTCCAGCC |
| | TATCTACTATAGCGGGTCTACATTCTACAAT | TGCATAGCGGTGTGCCTTCACGCTTTTCTGGA |
| | CCCTCCCTAAAATCCAGAGTTACCATTAGTG | TCTGGTAGCGGGACAGACTTCACGCTCACCAT |
| | AGGACACCAGCAAATCACAGTTCAGCTTAAA | TAGTTCTTTACAACCCGCCGACTTCGCAACCT |
| | GGTCACTAGCGTTACTGCCGCAGATACCGCT | ATTATTGTCAACAGGCGTATATCTCTCTTACA |
| | GTGTACTATTGCGCCCGTCACCCAGCCGGAT | TTTGGCCAAGGGACCAGGCTCGAAATCAAG |
| | ACAGTTCTTCTTGGTCTGCCTTTGATATATG | (SEQ ID NO: 1187) |
| | GGGTCAGGGAACTATGGTAACAGTGTCCTCC | |
| | (SEQ ID NO: 1186) | |
| SLC34A2 | CAGGTACAGCTGGTCGAATCCGGTGGCGGAT | GATATTCAGATGACACAGAGCCCTAGCTCCCT |
| Ab 9 | TGGTACAGCCAGGAAGGAGTTTACGGCTTTC | TTCCGCTTTTGTCGGCGACCGGGTGACTATTA |
| | CTGTAGTGGATCTGGTTTTACGAGCGGTGAT | CTTGCAGGGCCTCGCAGGACATAGGCAATTAC |
| | TATGCAGTGTCCTGGGTTAGACAGGCACCCG | CTGGCCTGGTATCAACAGACGCCTGAAAAAGT |
| | GGAAAGGCCTAGAGTGGGTTGGCTTCATCAG | CCCGAAACTGCTGATCTACGCTGCCTCGACTC |
| | AACCAAGCCTTACGGAGAAACCACAGAGTAC | TCCAGTCAGGCGTGCCCTCTCGCTTTTCTGGG |
| | GCTGCCAGTGTGAAAGGCCGATTCACCATTA | TCTGGATCAGGGACAGATTTCACTCTGACCAT |
| | GCCGCGACGACAGCAAGAGTATCGCCTATCT | CTCATCACTGCAACCAGAGGATGTCGCAACCT |
| | CCAGATGAACTCTCTCAAAGCTGAAGACACA | ATTACTGCCAAAACTACTATTCAGTTCCCTGG |
| | GCGGTGTTCTACTGTACCTTCATACCCGTCA | ACCTTTGGCCAAGGGACAAAGGTGGAGATCAA |
| | GCCGTTTTTTAGAGTGGTTGCCGTGGGGACA | G (SEQ ID NO: 1189) |
| | AGGGATTCCTGTGACTGTGTCCTCC (SEQ | |
| | ID NO: 1188) | |
| SLC34A2 | CAAGTGCAGCTCCTAGAGTCGGGCGGTGGGT | GATATTCAGATGACTCAGTCTCCTTCGTCACT |
| Ab 10 | TGGTTCAACCTGGAGCCAGCCTTCGGTTATC | GTCAGCGAGCGTAGGCGACCGGGTCACTATTA |
| | CTGTGCCGCAAGTGGTTTCACCTTTTCTACC | CTTGCAGGGCCTCTCAAGGAATCAGTAATTAC |
| | TACGCCATGACCTGGGTCAGGCAGGCACCTG | CTGGCTTGGTACCAGCAGCGCCCAGGGAAAGT |
| | GCAAAGGTTTAGAGTGGGTGAGTGGGATTAA | TCCAAAGCTGCTGATCTACGCCGCATCAACAC |
| | CGGCGGAGGTGACACCACCTATTATGCAGAT | TGAGATCAGGCGTGCCCTCTAGATTTAGCGGG |
| | AGTGTTAAAGGGCGATTCACCATATCCCGCG | TCAGGGTCTGGGACAGACTTCACCCTCACTAT |
| | ACAATTCCAAGAACACGCTTTACCTACAGAT | CAGCTCCCTCCAGCCCGAGGACGTGGCGACCT |
| | GAACAGCTTGAGGGCCGAAGATACGGCTGTC | ATTATTGCCAGAAGTACAATTCCGCTCCGCTG |
| | TACTATTGTGCTGTCCGTGGCTATACTTACG | ACATTTGGCGGAGGCACAAAGGTGGAAATCAA |
| | GCTATTTTTTCGATTACTGGGGACAAGGAAC | G (SEQ ID NO: 1191) |
| | GCTCGTGACAGTGTCCTCC (SEQ ID NO: | |
| | 1190) | |
| SLC34A2 | CAGGTTCAGTTGCAGGAATCGGGCCCAGGCC | GAGATCGTCCTGACACAGAGTCCCTCATCACT |
| Ab 11 | TGGTCAAACCGAGTGAAACGCTGTCCTTAAC | GAGTGCCTCTGTTGGGGATCGGCTAACCATTA |
| | CTGCACCGTATCTGGAGGCAGCATCTCCTCA | CATGTAGAGCCTCCCAGACCATCTCATCATAT |
| | AGCTCTTATTACTGGGGCTGGATTAGGCAGC | CTTAATTGGTATCAGCAAAAACCCGGGAAGGC |
| | CACCCGGCAAAGGCCTGGAGTGGATTGGTTC | ACCTAAAGTGCTGATCTACGCAGCATCCTCTC |
| | CTTGTACTACAGCGGGAGCACCTATTACAAC | TTCAGAGCGGCGTCCCATCTCGCTTTAGCGGT |
| | CCAAGCCTGAAGTCCCGAGTAACCATTAGTG | TCTGGGTCTGGGACTGACTTCACTCTCACTAT |
| | TGGACACTTCCAAGAACCAATTCAGTCTGAA | CTCATCATTGCAACCTGAGGACTTCGCTACAT |
| | GCTCAACTCTGTGACCGCTGCGGATACTGCC | ACTATTGCCAGCAGTCCTTCATCATACCTTAC |
| | GTGTACTATTGCACGAGGCACCCTAGAGGAA | ACGTTTGGACAAGGCACAAAGCTCGAGATAAA |
| | TTGCCGCTCGTTGGGGAAATTGGTTTGATCC | G (SEQ ID NO: 1193) |
| | CTGGGGTCAGGGTACACTCGTGACAGTGTCC | |
| | TCC (SEQ ID NO: 1192) | |
| SLC34A2 | CAAGTGCAACTGCAAGAATCAGGGCCTGGTT | GAAATTGTACTCACACAGAGTCCATCTTCCCT |
| Ab 12 | TGGTCAAGCCATCTGAGACACTTTCCTTAAC | TTCCGCAAGTGTCGGAGACAGAGTTACTATTA |
| | TTGTACGGTATCTGGTGGGTCAATCTCCTCC | CTTGCAGGGCCTCTCAGAGCATCTCTAGTTAC |
| | TCATCATATTACCGCGGGTGGATTAGGCAGC | CTGAACTGGTACCAGCAGAAACCGGGCAAAGC |
| | CACCCGGCAAAGGACTGGAGTGGATTGGAAG | ACCCAAGCTCCTAATCTACGCTGCCAGCTCTC |
| | TATATACTATTCCGGCTCAACGTACTACAAC | TGCAGTCTGGTGTGCCTAGTCGTTTTTCAGGA |
| | CCAAGCCTGAAGAGTCGGGTCACTATAAGTG | AGCGGCTCCGGAACAGATTTCACCCTCACCAT |
| | TGGACACTAGCAAGAATCAATTTTCCCTGAA | CAGCTCGTTACAGCCGGAGGACTTCGCCACCT |
| | ACTCTCGAGCGTTACCGCAGCCGATACCGCG | ATTACTGCCAGCAGAGCTATTCTACACCCTTC |
| | GTGTACTATTGTGCTAGACACCCTCGAGGGA | ACCTTTGGACCCGGCACAAAAGTGGATATCAA |
| | GCTATGGCGCTAATTTCGACTATTGGGGTCA | G (SEQ ID NO: 1195) |
| | GGGAACATTGGTTACTGTGTCCTCC (SEQ | |
| | ID NO: 1194) | |
| SLC34A2 | CAGGTTCAGTTAGTTGAGTCTGGTGGCGGGC | GACATTCAGATGACCCAATCTCCTTCTTCTCT |
| Ab 13 | TTGTCCAACCCGGTAGGAGCTTGCGGCTGAG | GAGTGCTTCGGTTGGGGATCGGGTCACTGTCA |
| | TTGCATAGGGAGTGGGTTCACGTTTGGGGAA | CCTGTCGCGCTAACCAGGACATTAACAATTAC |
| | TATGCCATGAGTTGGGTGAGGCAGGCACCTG | CTGGCTTGGTACCAGCAGACACCCGGCAAAGT |
| | GCAAAGGGCTAGAGTGGGTGGGGTTCATCAG | ACCAAAACTCCTTATTTACGCCGCAAGCACTC |
| | AACCAAGCCATACGGTGGCACCACTGAATTT | TGCAGAGCGGAGTACCAAGTCGATTCAGCGGA |
| | GCGGCAAGCGTGAAGGGAAGATTCACGATGT | TCAGGCTCTGGGACTGATTTCACACTGACCAT |
| | CCCGCGATGACTCCAAATCAATCGCCTATCT | CTCCTCACTGCAACCTGAGGACGTCGCCACGT |
| | CGAGATGAACTCCCTCAAGACCGAGGACACT | ACTATTGCCAGAATTATTACAGCGTGCCCTGG |
| | GCGGTGTACTATTGTACACTCATACCCGCCC | ACCTTTGGTCAGGGAACAAAGCTGGAAATCAA |
| | TTCGTTTTCTGGAGTGGTTGCCCTGGGGACA | G (SEQ ID NO: 1197) |
| | AGGCACATTGGTGACAGTGTCCTCC (SEQ | |
| | ID NO: 1196) | |
| SLC34A2 | CAAGTGCAACTGCAAGAGAGCGGACCAGGGC | GAGATCGTTCTGACCCAGTCACCTGATTTCCA |
| Ab 14 | TGGTGAAACCCTCTGAGACCCTTTCCCTGAC | GAGCGTAACACCTAAAGAATCTGTGACTATAA |
| | CTGTACGGTTTCAGGAGGGTCCGTGTCCTCC | CTTGTCGGGCAAGCCAGTCTGTCGGATCAGGC |
| | GGTAGTTATTACTGGTCATGGATTAGACAGC | CTCCACTGGTACCAGCAGAAGCCCGACCAGAG |
| | CTCCGGGTAAAGGGTTAGAGTGGATTGGCTA | TCCCAAGCTCCTAATCAAATATGCCAGTCAGA |
| | CATATTCTACTCCGGGTCTACGTACTATAAC | GCTTCTCAGGAGTCCCAAGTCGATTTTCAGGG |
| | CCTAGTTTGAAGTCGCGCGTTACCATTAGCG | AGTGGCAGCGGAACAGATTTCACATTGACAAT |
| | TGGACACTAGCAAGAACCAATTCTCCCTTAA | CAATTCCCTGGAAGCCGAGGACGCGGCCACAT |
| | GCTCACCAGTGTGACAGCCGCAGACACCGCT | ATTATTGCCTCCAATCTAGCAGCCTGCCCTGG |
| | GTCTACTTTTGCGCTAGGTGGATGACCACTG | ACTTTTGGGCAAGGCACGAAGGTGGAAATCAA |
| | TCAAAGGCTACTTTGATTATTGGGGTCAGGG | G (SEQ ID NO: 1199) |
| | AACCCTGGTAACTGTCTCCTCC (SEQ ID | |
| | NO: 1198) | |
| SLC34A2 | CAGGTTCAGTTGCAGGAAAGCGGGCCCGGGC | GATATTCAGATGACCCAAAGCCCTAGTTCCCT |
| Ab 15 | TGGTCAAACCATCAGAAACCTTGTCCTTAAC | GTCCGCAAGTGTTGGGGACCGGGTGACTATTA |
| | CTGTACCGTAAGCGGCGGAAGTGTGTCCTCC | CATGCCGAGCTTCTCAAAGCATCAGCTCATAC |
| | GCTTCATATTATTGGTCGTGGATAAGGCAGC | CTGAACTGGTACCAGCAGAAACCCGGCAAGGC |
| | CACCCGGAAAAGGGCTCGAGTACATTGGATA | ACCTAAGTTCCTGATTAGCCCAGCCTCTAGCC |
| | CATCTACTATTCAGGATCTACTTACTACAAC | TGCAGTCTGGCGTACCCAGTCGCTTTTCTGGG |
| | CCAAGCCTGAAGTCAAGAGTGACCATCTCCA | TCCGGCTCTGGCACGGATTTCACGCTAACAAT |
| | TTGATACCTCCAAGAATCAGTTCAGTCTTAA | CAGCTCGCTTCAGCCGGAGGACTTTGCCACTT |
| | TCTCAGGAGTGTCACTGCTGCCGACACAGCG | ATTATTGCCAGCAATCATATAGCATACCTTGG |
| | GTGTACTATTGCGCCAGATACATCGTCGGTC | ACATTTGGCCAAGGGACAAAGGTGGAAATCAA |
| | GTCCCGGCTTCAACTGGTTTGACCCGTGGGG | G (SEQ ID NO: 1201) |
| | TCAGGGTACACTCGTGACTGTGTCCTCC | |
| | (SEQ ID NO: 1200) | |
| SLC34A2 | CAAGTGCAGCTCCAAGAATCAGGGCCCGGGT | GATATTCAGATGACTCAGAGTCCCTCATCCCT |
| Ab 16 | TGGTTAAACCAAGTGAAACCCTTTCCCTGAC | TTCCGCTTCTGTCGGCGACCGCGTTACAATCA |
| | TTGCACCGTTAGCGGAGATTCTGTGTCCTCC | CCTGTAGGGCCTCGCAGACAATCAGTTCGTAC |
| | GTGTCCTATTACTGGAGCTGGATACGACAGC | CTGAACTGGTATCAACAGAAACCTGGGAAGGC |
| | CACCTGGCAAGGGACTGGAGTGGATTGGCTA | TCCTAAACTCCTCATAAGTCCTGCCTCAAATT |
| | CATCTGGTACAGCGGCAGCACCTATTACAAC | TACAGTCTGGGGTCCCAAGCAGATTTTCTGGA |
| | CCAAGTCTAAAGTCTCGGGTGACAATTTCCA | TCAGGGTCTGGGACGGATTTCACCCTGACTAT |
| | TTGACACTTCAAAGAATCAGTTCAGCCTGAA | CAGCTCGCTGCAGCCGGAGGACTTTGCCACGT |
| | GTTAAGGAGTGTGACCGCAGCCGACACTGCG | ATTATTGCCAGCAATCATACATCATTCCCTGG |
| | GTGTACTATTGTGCAAGATACATCGTAGGCC | ACCTTTGGCCAGGGAACAAAGGTCGAGATCAA |
| | GTCCCGGTTTCAACTGGTTCGATCCCTGGGG | G (SEQ ID NO: 1203) |
| | TCAGGGAACATTGGTGACAGTATCCTCC | |
| | (SEQ ID NO: 1202) | |

In some embodiments, the V_{H} of the antibody or antigen-binding fragment that binds to SLC34A2 is encoded by a nucleic acid comprising a sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the sequence set forth in SEQ ID NOs: 1110, 1112, 1114, 1178, 1180, 1182, 1184, 1186, 1188, 1190, 1192, 1194, 1196, 1198, 1200, or 1202, optionally, wherein the V_{H} CDRs are identical to those encoded by SEQ ID NOs: 1110, 1112, 1114, 1178, 1180, 1182, 1184, 1186, 1188, 1190, 1192, 1194, 1196, 1198, 1200, or 1202, respectively. In some embodiments the V_{L} of the antibody or antigen-binding fragment that binds to SLC34A2 is encoded by a nucleic acid comprising a sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the sequence set forth in SEQ ID NO: 999, 1111, 1113, 1115, 1179, 1181, 1183, 1185, 1197, 1189, 1191, 1193, 1195, 1197, 1199, 1201, or 1203, optionally, wherein the V_{L} CDRs are identical to those encoded by SEQ ID NOs: 999, 1111, 1113, 1115, 1179, 1181, 1183, 1185, 1197, 1189, 1191, 1193, 1195, 1197, 1199, 1201, or 1203, respectively.

Table 12 provides exemplary nucleic acid sequences encoding scFvs that bind to SLC34A2. In some embodiments, the antibody or antigen-binding fragment that binds to SLC34A2 comprises an scFv encoded by a nucleic acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to a nucleic acid sequence set forth in Table 12, and optionally comprising V_{H} and V_{L} CDR sequences that are 100% identical to those thereof.

TABLE 12
SLC34A2 scFv Nucleotide Sequences

| Clone | scFv Sequence |
|---|---|
| SLC34A2 | GAAATCGTCCTGACACAGTCTCCAGATTTTCAGAGCGTGACGCCAAAGGAGAAAGTGACAATTACAT |
| Ab 1 scFv | GCCGGGCATCTCAGTCTGTTGGGTCTGGGTTGCATTGGTATCAGCAAAAGCCCGACCAGTCACCCAA |
| (VL-VH) | ACTGCTCATCAAATATGCAAGCCAGAGTTTTTCAGGCGTACCTTCACGATTTAGCGGAAGTGGTTCT |
| (SEQ ID | GGCACTGACTTCACCTTGACGATTAATAGCCTGGAAGTAGAAGACGCTGCCACTTTCTACTGCCTGC |
| NO: 1116 | AAAGTAGCTCCCTGCCCTGGACTTTTGGGCAGGGTACTAAGGTCGAGATCAAGGGCTCGACAAGCGG |
| | AAGTGGCAAACCGGGCAGCGGCGAGGGAAGCACCAAGGGACAAGTGCAACTGCAAGAGTCTGGACCC |
| | GGGCTGGTGAAACCAAGCGAGACATTATCCCTCACTTGTACCGTGTCAGGCGGTAGTGTGTCCTCCG |
| | GGAATTTCTACTGGAGTTGGATACGCCAGCCTCCTGGGAAGGGCCTTGAATGGATTGGCTACATCTA |
| | CTATTCAGGCTCCACCTACTACAACCCGTCTTTGAAGTCAAGGGTTACGATAAGCGTCGATACCTCC |
| | AAAAACCAATTCTCCCTAAAGCTCAGATCGTTAACTGCCGCTGATACCGCGGTGTACTATTGTGCCC |
| | GTTGGATGACCAAAGTTAAGGGTTATTTCGACTATTGGGGACAAGGGACACTTGTCACCGTGTCCTC |
| | C |
| SLC34A2 | CAGGTTCAACTGCAAGAGTCTGGGCCCGGATTGGTGAAGCCAAGTGAAACCCTTTCCCTGACTTGTA |
| Ab 1 scFv | CGGTATCCGGCGGTTCGGTGTCCTCCGGGAATTTCTACTGGTCATGGATTAGACAGCCACCTGGTAA |
| (VH-VL) | AGGGCTGGAATGGATTGGGTACATCTACTACTCTGGAAGCACTTACTATAACCCATCTCTTAAAAGT |
| SEQ ID | AGAGTCACCATAAGTGTGGACACAAGCAAGAACCAGTTCAGCCTGAAGCTGCGTTCTTTAACCGCCG |
| NO: 1209 | CGGACACAGCCGTGTATTACTGCGCTAGGTGGATGACCAAAGTAAAGGGCTATTTCGATTATTGGGG |
| | ACAAGGGACATTGGTCACAGTGTCCTCCGGCTCAACTAGCGGTAGTGGTAAACCTGGCAGCGGCGAG |
| | GGAAGTACGAAAGGCGAGATAGTCCTGACTCAGTCTCCAGATTTTCAGTCGGTGACGCCTAAGGAGA |
| | AGGTTACTATTACCTGCCGAGCATCACAGTCTGTGGGCAGCGGGCTCCACTGGTATCAACAGAAACC |
| | GGATCAGAGCCCTAAGCTCCTCATCAAATATGCCAGTCAGTCATTTTCTGGAGTGCCCTCCCGGTTT |
| | TCCGGCAGCGGCTCAGGAACCGACTTCACCCTGACAATCAATAGCCTCGAGGTCGAAGACGCAGCCA |
| | CCTTCTACTGTCTACAGTCTTCATCCTTACCCTGGACTTTTGGACAAGGGACAAAGGTTGAAATCAA |
| | G |
| SLC34A2 | GAGATAGTCCTGACCCAGTCACCCGATTTCCAGAGTGTTACTCCTAAGGAGAAAGTGACTATAACAT |
| Ab 2 scFv | GCCGGGCATCTCAGTCTGTCGGAAGCGGGCTACATTGGTACCAGCAGAAGCCTGACCAGAGCCCGAA |
| (VL-VH) | ACTGCTCATCAAATATGCCTCCCAGTCGTTTTCTGGCGTGCCCTCTCGCTTTTCCGGAAGCGGATCT |
| SEQ ID | GGCACAGACTTCACCTTGACCATCAATAGCCTGGAAACTGAGGACGCCGCTACGTATTTCTGCCAGC |
| NO: 1117 | AGTCCTCCAGTCTGCCTTGGACATTTGGTCAGGGAACGAAGGTGGAGATCAAGGGTTCAACATCAGG |
| | GAGCGGGAAACCGGGCTCTGGCGAGGGCTCAACAAAGGGACAAGTGCAACTGCAAGAATCGGGACCC |
| | GGGCTGGTTAAACCAAGTGAAACCCTTTCCCTCACTTGTACCGTAAGCGGCGGTAGCGTGTCCTCTG |
| | GTAGCTACTATTGGAGTTGGATTAGGCAGGCGCCAGGGAAAGGCCTCGAATGGATTGGGTATATCTA |
| | CTACAGCGGCAGTAACTACTACAACCCATCATTGAAGTCTAGAGTGACAATTAGTGTCGATACCTCT |
| | AAAAATCAATTCTCACTTAAGCTGCGAGCTGTAACCGCCGCAGACACTGCGGTGTACTATTGTGCCC |
| | GTTGGATGACCACTATCAAGGGCTACTTCGATTATTGGGGACAAGGGACATTAGTTACGGTGTCCTC |
| | C |
| SLC34A2 | CAGGTCCAACTGCAAGAATCGGGACCCGGGCTGGTTAAGCCTTCCGAAACGTTGAGTCTGACCTGTA |
| Ab 2 scFv | CCGTATCTGGAGGCTCGGTGTCCTCCGGCAGTTATTATTGGAGCTGGATTCGGCAGGCACCAGGGAA |
| (VH-VL) | AGGGCTGGAATGGATTGGATACATCTACTACTCAGGATCAAACTACTATAATCCATCCCTGAAGAGT |
| SEQ ID | AGGGTCACAATCAGTGTGGACACCAGCAAAAACCAATTCTCCTTGAAACTTAGAGCCGTCACTGCGG |
| NO: 1210 | CAGATACCGCTGTGTACTATTGCGCGCGTTGGATGACTACCATCAAAGGCTACTTCGATTACTGGGG |
| | TCAGGGTACACTCGTTACCGTGTCCTCCGGAAGCACATCTGGTTCTGGGAAACCTGGCTCTGGCGAG |
| | GGTTCAACGAAGGGCGAAATAGTATTAACGCAGTCTCCAGACTTTCAGTCAGTGACACCTAAGGAGA |
| | AAGTTACTATAACCTGCCGAGCCTCACAATCTGTGGGAAGTGGCCTACATTGGTATCAGCAAAAGCC |
| | CGACCAGAGCCCAAAGCTCCTCATCAAATATGCTTCACAGAGCTTCAGCGGTGTGCCCTCTCGCTTC |
| | TCCGGGTCTGGCTCCGGCACTGACTTTACACTGACTATTAATAGCCTTGAGACCGAGGATGCCGCAA |
| | CCTACTTTTGTCAACAAAGCAGCAGCTTACCGTGGACATTTGGACAAGGGACAAAGGTCGAGATCAA |
| | G |
| SLC34A2 | GATATTCAGATGACACAGAGCCCAAGTTCACTGTCAGCGAGTGTCGGAGATCGGGTTACTATAACCT |
| Ab 3 scFv | GCCGAGCATCTCAGGATATCAATAATTACCTGGCCTGGTATCAGCAAAAACCCGGCAAAGTCCCGAA |
| (VL-VH) | GCTCCTGATATACGCAGCTTCAACACTGCAAAGCGGTGTGCCCTCGCGCTTTAGCGGCTCTGGCAGC |
| SEQ ID | GGGACAGATTTCACCCTGACGATTTCCTCCCTTCAACCCGAGGACGTGGCCACTTACTACTCCCTCA |
| NO: 1118 | ACTATTACTCTGTACCCTGGACCTTTGGCCAAGGGACAAAGGTGGAAATCAAGGGCTCTACTAGCGG |
| | TTCAGGGAAACCTGGGAGTGGAGAGGGCTCGACCAAGGGACAGGTCCAGCTCGTGGAGTCTGGCGGT |
| | GGGCTGGTTCAGCCAGGGAGGAGTTTGCGGCTTTCCTGTACGGGAAGCGGATTCACCTTTGGTGACT |
| | ATGCCATGAACTGGGTCAGGCAGGCTCCTGGAAAAGGCCTAGAGTGGGTGGGTTTCATCAGAACCAA |
| | GCCATATGGCGGCACTACAGAATATGCAGCCAGCGTAAAAGGGAGATTCACGTTCAGCCGCGACGAC |
| | TCTAAGTCAATAGCTTACCTTCAGATGAACTCCCTCAAGACCGAAGACACCGCCGTGTACTATTGTA |
| | CTATGATCCCTGTGCTGCGTTTTTTAGAGTGGTTGCCTTGGGGACAAGGGACATTAGTTACTGTGTC |
| | CTCC |
| SLC34A2 | CAAGTGCAGCTGGTCGAATCTGGAGGCGGCCTGGTCCAGCCCGGAAGGAGCTTGCGACTGTCATGCA |
| Ab 3 scFv | CCGGTTCCGGGTTCACGTTTGGGGATTATGCCATGAACTGGGTCAGGCAGGCTCCTGGAAAGGGACT |
| (VH-VL) | GGAGTGGGTGGGGTTCATCAGAACCAAGCCCTATGGTGGAACTACCGAATATGCCGCCTCTGTTAAA |
| SEQ ID | GGAAGATTCACCTTTAGCCGCGATGACTCCAAAAGCATCGCATACCTCCAGATGAACTCACTCAAAA |
| NO: 1211 | CAGAGGACACCGCGGTGTATTACTGCACAATGATCCCAGTCCTGCGGTTCCTTGAGTGGCTGCCGTG |
| | GGGACAAGGCACACTCGTGACAGTGTCCTCCGGTTCTACTAGCGGAAGTGGCAAACCGGGTTCTGGG |
| | GAGGGCTCGACCAAGGGCGATATTCAGATGACTCAGAGTCCCTCATCCCTATCAGCAAGTGTTGGGG |
| | ACCGCGTGACCATTACATGTCGGGCCTCCCAGGACATCAATAATTACCTAGCTTGGTATCAACAGAA |
| | GCCTGGCAAAGTACCAAAACTCTTGATATACGCCGCTTCGACTCTTCAGAGCGGTGTCCCTAGTCGT |
| | TTTTCAGGCAGCGGCTCTGGCACTGATTTCACGTTAACAATATCCAGCTTACAGCCAGAGGACGTAG |
| | CAACTTACTATTCTCTGAACTACTACAGCGTTCCCTGGACCTTTGGGCAAGGGACAAAGGTGGAAAT |
| | TAAG |
| SLC34A2 | GATATTCAGATGACTCAATCGCCCTCTTCCCTGAGCGCCAGTGTCGGAGATCGAGTAACCATCACTT |
| Ab 4 scFv | GTAGGGCTAGTCAAGGAATCTCTAACTATCTGGCCTGGTACCAGCAGAAGCCTGGGAAAGTTCCGAA |
| (VL-VH) | CCTTCTCATCTACACAGCAAGCACCCTCCAGTCTGGGGTTCCTAGTAGGTTTTCAGGGTCTGGCAGC |
| SEQ ID | GGTACTGACTTCACGCTGACTATTTCATCCTTGCAACCCGAGGATGTTGCAACTTATTATTGCCAGA |
| NO: 1212 | AGTACAATAGCGCTCCATTCACCTTTGGCCCAGGGACAAAGGTGGACATCAAGGGCTCGACATCTGG |
| | TAGCGGCAAACCTGGCTCTGGCGAGGGAAGTACCAAGGGACAGGTCCAGCTGCTGGAAAGCGGTGGT |
| | GGGCTGGTCCAACCTGGAGGATCATTGCGGCTGTCCTGCGCCGCCAGTGGGTTCACCTTTAGCTCCT |
| | ACGCCATGAATTGGGTGCGCCAGGCTCCCGGCAAAGGCCTAGAGTGGGTGTCTGCCATTAGCGGCGG |
| | AGTCGGGAATACCTACTATGCGGACAGCGTGAAAGGGCGGTTTACAATATCACGCGACAACTCCAAA |
| | AACACCTTATACTTACAGATGAACTCACTTAGAGCTGAAGATACCGCAGTGTACTATTGTACGAAGG |
| | ACGGTCCACTCTGGGGCAATTATTTCGATTACTGGGGACGTGGCACACTCGTGACAGTGTCCTCC |
| SLC34A2 | CAAGTGCAGCTGCTGGAATCTGGCGGCGGGCTGGTGCAACCTGGAGGGAGCTTGCGGTTATCATGCG |
| Ab 4 scFv | CCGCCTCTGGCTTCACCTTCTCCTCCTATGCCATGAATTGGGTTAGGCAGGCTCCTGGGAAAGGTCT |
| (VH-VL) | GGAGTGGGTAAGCGCTATTAGTGGTGGAGTGGGAAACACATATTATGCCGATTCTGTGAAGGGACGC |
| SEQ ID | TTTACAATCTCGAGAGACAACAGCAAAAATACTCTGTACCTTCAAATGAACTCCCTCCGAGCCGAGG |
| NO: 1213 | ATACCGCAGTGTACTATTGCACAAAGGACGGGCCACTTTGGGGCAACTATTTTGATTACTGGGGCAG |
| | AGGAACATTGGTAACCGTGTCCTCCGGGAGTACATCTGGCAGCGGGAAACCCGGCTCTGGGGAAGGT |
| | TCAACCAAGGGCGACATTCAGATGACTCAGTCACCTAGCTCACTGTCAGCGTCAGTCGGAGATCGGG |
| | TTACTATTACGTGTAGGGCTAGTCAGGGTATCAGCAATTACCTCGCCTGGTACCAGCAGAAACCCGG |
| | CAAAGTCCCAAACCTGCTCATCTACACTGCAAGCACCCTACAGAGCGGTGTTCCATCCCGTTTTTCT |
| | GGATCGGGATCTGGGACGGATTTCACCCTCACCATATCCTCCTTACAACCCGAGGACGTCGCAACTT |
| | ATTACTGTCAGAAGTACAATAGTGCTCCCTTCACGTTTGGCCCGGGCACAAAGGTGGACATCAAG |
| SLC34A2 | GATATTCAAATGACTCAGAGTCCTTCTTCACTGTCAGCCTCTGTTGGCGACCGGGTCACTATAACTT |
| Ab 5 scFv | GTAGGGCTTCGCAGTCAATCTCCTCATACCTGAACTGGTACCAGCAGAAGCCCGGCAAAGCGCCGAA |
| (VL-VH) | ACTTTTGATATATGCAGCCAGTTCCTTACTGACCGGAGTACCTAGTCGCTTCACCGGCAGCGGGAGC |
| SEQ ID | GGGACAGACTTCACGCTCACGATTAGTTCCTTGCAGCCCGAGGACTTTGGAACCTATTACTGCCAAC |
| NO: 1214 | AGTCTTATAGCCCACCTCTTACGTTTGGCGGCGGCACAAAGGTGGAAATCAAGGGTTCTACAAGCGG |
| | ATCTGGGAAACCCGGGAGCGGAGAAGGTTCTACCAAGGGACAGGTCCAACTACAGGAGTCGGGCCCT |
| | GGGCTGGTGAAGCCATCTGAAACCCTGTCACTCACTTGCTCTGTTAGTGGTGGTTCAATCTCCTCCA |
| | GCTCATATTACTGGGGCTGGATTAGACAGCCACCCGGAAAAGGGTTAGAGTGGATAGGCAGTATCGA |
| | CTACAGCGGTAGCACCTATTACAACCCAAGCCTCAAGTCCCGAGTCACCATCAGTATCGATACAAGC |
| | AAGAATCAGTTCAGCCTGAGGCTGTCCTCCGTGACAGCCGCTGAGAAAGCCGTGTACTATTGTGCAA |
| | GACATGGCCGTGGGACTATTGGATATTTTGATTACTGGGGACAAGGGACACTCGTGACCGTGTCCTC |
| | C |
| SLC34A2 | GATATACAGATGACTCAGTCGCCCAGCTCATTGAGTGCGAGTGTCGGAGATCGCGTGACCATTGCGT |
| Ab 6 scFv | GCAGGGCTTCACAAGTGATTAGCAATTACCTGGCCTGGTATCAGCAAAAACCGGGCAAGGTGCCTAA |
| (VL-VH) | GCTGCTGATATACGTCGCCTCGACTCTCCAGAGTGGCGTGCCTTCTAGATTTTCAGGGAGTGGCAGC |
| SEQ ID | GGAACCGATTTCACCCTGACCATCTCATCACTGCAACCAGAGGACGTAGCCACCTATTACTGCCAGA |
| NO: 1216 | ATTATAACAGCGCACCCTGGACATTTGGGCAGGGTACAAAGCTGGAAATCAAGGGTTCTACATCTGG |
| | GTCTGGGAAACCTGGGTCTGGGGAGGGCTCCACCAAGGGACAGGTTCAGCTTCTTGAAAGTGGCGGC |
| | GGATTAGTGCAGCCAGGAGGTTCTTTGCGGCTTTCATGTGCAGCCAGTGGCTTCACCTTCTCCTCCT |
| | CTGCTATGGCCTGGGTTCGACAGGCTCCCGGAAAAGGGCTAGAGTGGGTTAGCGCTATCTCCTCCTC |
| | TGGCGATAATACTTACTACGCCGACAGCGTAAAAGGCAGGTTCACGATTAGCAGAGACACTAGCAAA |
| | AACACGCTCAGCCTCCAGATGTCCTCCCTGCGTGCCGAGGACACTGCAATCTACTATTGTGCAAAGC |
| | AGGGAACAAACTGGGGCTTATACTTTGACTATTGGGGTCAAGGAACATTGGTCACAGTGTCCTCC |
| SLC34A2 | CAGGTCCAGCTTCTGGAAAGCGGCGGCGGGCTGGTACAGCCAGGAGGCTCCTTGCGGTTATCATGTG |
| Ab 6 scFv | CCGCTAGTGGATTCACCTTTTCTAGCTCTGCAATGGCCTGGGTGCGCCAGGCACCGGGAAAAGGTCT |
| (VH-VL) | GGAGTGGGTCAGCGCAATATCAAGCTCTGGTGACAACACCTACTATGCCGATAGTGTCAAGGGAAGG |
| SEQ ID | TTCACCATCTCGAGAGACACATCTAAAAATACTCTTAGCCTTCAGATGTCCTCCCTCCGAGCTGAGG |
| NO: 1217 | ACACAGCGATCTACTATTGCGCTAAGCAAGGAACAAATTGGGGACTCTACTTTGATTATTGGGGTCA |
| | GGGAACACTCGTGACTGTGTCCTCCGGCTCAACATCTGGGAGTGGCAAACCCGGGAGCGGCGAAGGG |
| | AGCACCAAGGGCGACATTCAGATGACCCAGTCTCCTAGTTCACTGTCAGCCTCTGTGGGCGACAGAG |
| | TGACCATTGCATGCAGGGCCTCTCAGGTTATTAGTAACTACTTGGCTTGGTACCAGCAGAAACCTGG |
| | GAAGGTTCCCAAACTGCTGATATACGTAGCTTCGACTCTACAGTCAGGCGTGCCATCCCGTTTTAGC |
| | GGAAGCGGTAGCGGCACTGATTTCACGCTCACCATCTCCTCCCTGCAACCCGAGGATGTTGCCACGT |
| | ATTATTGTCAAAACTACAATAGTGCGCCTTGGACTTTCGGGCAAGGGACAAAGTTAGAGATCAAG |
| SLC34A2 | GAGATTGTCTTGACCCAGTCTCCCTCTTCACTGTCGGCCTCTGTTGGGGACCGCGTAACCATTACCT |
| Ab 7 scFv | GCCGGGCCTCTCAGTCAATCAGTTCTTACCTGAATTGGTACCAGCAGAAGCCAGGGCGGGCACCCGA |
| (VL-VH) | ACTGCTGATCTACGCAGCCAGCAGCCTGCAGTCTGGGGTGCCCTCGAGGTTTAGCGGCTCAGGAAGC |
| SEQ ID | GGAACAGACTTCACACTCACAATCTCCTCCCTCCAACCCGAGGACTTCGCCACATACTATTGCCAGC |
| NO: 1218 | AATCATTTTCCTCCCTAACCTTCGGCCAGGGTACTAGGCTCGAAATCAAGGGCTCAACTTCTGGGAG |
| | CGGCAAACCTGGTAGCGGAGAGGGTTCTACCAAGGGACAGGTCCAACTGCAAGAATCGGGCCCTGAG |
| | TTAGTTAAACCTAGCGAAACGTTGTCCATTACATGTACCGTGAGTGGTGGCTCTATCTCCTCCAGAA |
| | GTTATTATTGGGGCTGGATTAGACAGCCACCTGGGAAAGGGCTCGAGTGGATCGGGTCCATATACTA |
| | CGGAGGCAGCACCTACTATAATCCAAGTCTGAAGAGTCGAGTAACTATATCAGCGGATACGAGCAAG |
| | AACCAGTTTAGCCTTAAACTTAACAGTGTGACCGCCGCTGATACTGCTGTGTTCTATTGTGTCCGCC |
| | ACCCGGCTGGATATAGCACTCGTTGGAGTGCATTTGATATTTGGGGACAAGGCACAATGGTGACTGT |
| | GTCCTCC |
| SLC34A2 | GAGATTGTGCTGACACAGAGCCCTTCTTCGCTGAGTGCCAGTGTCGGAGATCGGGTCACAATTACCT |
| Ab 8 scFv | GTAGGGCATCACAGTCAATCTCCTCCTACCTGAACTGGTACCAGTTGAAGCCTGGCAAGGCTCCCAA |
| (VL-VH) | ACTTTTGATATACGCTGCTTCCAGCCTGCATAGCGGTGTGCCTTCACGCTTTTCTGGATCTGGTAGC |
| SEQ ID | GGGACAGACTTCACGCTCACCATTAGTTCTTTACAACCCGCCGACTTCGCAACCTATTATTGTCAAC |
| NO: 1219 | AGGCGTATATCTCTCTTACATTTGGCCAAGGGACCAGGCTCGAAATCAAGGGCTCGACTAGCGGTTC |
| | AGGGAAGCCAGGCAGCGGCGAAGGCTCAACCAAGGGACAAGTGCAGCTCCAGGAGAGCGGGCCAGGG |
| | CTGGTGAAACCAAGTGAAACGCTGTCCCTGACTTGCACGGTTAGCGGCGGCTCGATCTCCTCCAGAT |
| | CATATTACTGGGGATGGATCCGACAGCCTCCCGGCAAAGGGCCGGAGTGGATTGGAAGTATCTACTA |
| | TAGCGGGTCTACATTCTACAATCCCTCCCTAAAATCCAGAGTTACCATTAGTGAGGACACCAGCAAA |
| | TCACAGTTCAGCTTAAAGGTCACTAGCGTTACTGCCGCAGATACCGCTGTGTACTATTGCGCCCGTC |
| | ACCCAGCCGGATACAGTTCTTCTTGGTCTGCCTTTGATATATGGGGTCAGGGAACTATGGTAACAGT |
| | GTCCTCC |
| SLC34A2 | GATATTCAGATGACACAGAGCCCTAGCTCCCTTTCCGCTTTTGTCGGCGACCGGGTGACTATTACTT |
| Ab 9 scFv | GCAGGGCCTCGCAGGACATAGGCAATTACCTGGCCTGGTATCAACAGACGCCTGAAAAAGTCCCGAA |
| (VL-VH) | ACTGCTGATCTACGCTGCCTCGACTCTCCAGTCAGGCGTGCCCTCTCGCTTTTCTGGGTCTGGATCA |
| SEQ ID | GGGACAGATTTCACTCTGACCATCTCATCACTGCAACCAGAGGATGTCGCAACCTATTACTGCCAAA |
| NO: 1220 | ACTACTATTCAGTTCCCTGGACCTTTGGCCAAGGGACAAAGGTGGAGATCAAGGGATCTACAAGCGG |
| | GAGTGGCAAGCCAGGGAGCGGAGAGGGTAGCACCAAGGGTCAGGTACAGCTGGTCGAATCCGGTGGC |
| | GGATTGGTACAGCCAGGAAGGAGTTTACGGCTTTCCTGTAGTGGATCTGGTTTTACGAGCGGTGATT |
| | ATGCAGTGTCCTGGGTTAGACAGGCACCCGGGAAAGGCCTAGAGTGGGTTGGCTTCATCAGAACCAA |
| | GCCTTACGGAGAAACCACAGAGTACGCTGCCAGTGTGAAAGGCCGATTCACCATTAGCCGCGACGAC |
| | AGCAAGAGTATCGCCTATCTCCAGATGAACTCTCTCAAAGCTGAAGACACAGCGGTGTTCTACTGTA |
| | CCTTCATACCCGTCAGCCGTTTTTTAGAGTGGTTGCCGTGGGGACAAGGGATTCCTGTGACTGTGTC |
| | CTCC |
| SLC34A2 | GATATTCAGATGACTCAGTCTCCTTCGTCACTGTCAGCGAGCGTAGGCGACCGGGTCACTATTACTT |
| Ab 10 scFv | GCAGGGCCTCTCAAGGAATCAGTAATTACCTGGCTTGGTACCAGCAGCGCCCAGGGAAAGTTCCAAA |
| (VL-VH) | GCTGCTGATCTACGCCGCATCAACACTGAGATCAGGCGTGCCCTCTAGATTTAGCGGGTCAGGGTCT |
| SEQ ID | GGGACAGACTTCACCCTCACTATCAGCTCCCTCCAGCCCGAGGACGTGGCGACCTATTATTGCCAGA |
| NO: 1221 | AGTACAATTCCGCTCCGCTGACATTTGGCGGAGGCACAAAGGTGGAAATCAAGGGCTCTACAAGTGG |
| | TAGCGGAAAACCCGGTAGCGGCGAAGGGAGCACCAAGGGACAAGTGCAGCTCCTAGAGTCGGGCGGT |
| | GGGTTGGTTCAACCTGGAGCCAGCCTTCGGTTATCCTGTGCCGCAAGTGGTTTCACCTTTTCTACCT |
| | ACGCCATGACCTGGGTCAGGCAGGCACCTGGCAAAGGTTTAGAGTGGGTGAGTGGGATTAACGGCGG |
| | AGGTGACACCACCTATTATGCAGATAGTGTTAAAGGGCGATTCACCATATCCCGCGACAATTCCAAG |
| | AACACGCTTTACCTACAGATGAACAGCTTGAGGGCCGAAGATACGGCTGTCTACTATTGTGCTGTCC |
| | GTGGCTATACTTACGGCTATTTTTTCGATTACTGGGGACAAGGAACGCTCGTGACAGTGTCCTCC |
| SLC34A2 | GAGATCGTCCTGACACAGAGTCCCTCATCACTGAGTGCCTCTGTTGGGGATCGGCTAACCATTACAT |
| Ab 11 scFv | GTAGAGCCTCCCAGACCATCTCATCATATCTTAATTGGTATCAGCAAAAACCCGGGAAGGCACCTAA |
| (VL-VH) | AGTGCTGATCTACGCAGCATCCTCTCTTCAGAGCGGCGTCCCATCTCGCTTTAGCGGTTCTGGGTCT |
| SEQ ID | GGGACTGACTTCACTCTCACTATCTCATCATTGCAACCTGAGGACTTCGCTACATACTATTGCCAGC |
| NO: 1222 | AGTCCTTCATCATACCTTACACGTTTGGACAAGGCACAAAGCTCGAGATAAAGGGATCGACTAGCGG |
| | CAGCGGGAAACCTGGTAGCGGCGAAGGAAGTACCAAGGGACAGGTTCAGTTGCAGGAATCGGGCCCA |
| | GGCCTGGTCAAACCGAGTGAAACGCTGTCCTTAACCTGCACCGTATCTGGAGGCAGCATCTCCTCAA |
| | GCTCTTATTACTGGGGCTGGATTAGGCAGCCACCCGGCAAAGGCCTGGAGTGGATTGGTTCCTTGTA |
| | CTACAGCGGGAGCACCTATTACAACCCAAGCCTGAAGTCCCGAGTAACCATTAGTGTGGACACTTCC |
| | AAGAACCAATTCAGTCTGAAGCTCAACTCTGTGACCGCTGCGGATACTGCCGTGTACTATTGCACGA |
| | GGCACCCTAGAGGAATTGCCGCTCGTTGGGGAAATTGGTTTGATCCCTGGGGTCAGGGTACACTCGT |
| | GACAGTGTCCTCC |
| SLC34A2 | GAAATTGTACTCACCCAAAGCCCAAGCTCCCTGTCCGCCTCTGTTGGTGATCGGGTCACAATAACAT |
| Ab 12 scFv | GCAGGGCGTCACAGAGCATCTCCTCCTACCTGAACTGGTACCAGCAGAAGCCTGGCAAGGCACCGAA |
| (VL-VH) | GCTGCTGATATACGCCGCCTCTAGTCTACAGTCTGGGGTGCCCTCTCGCTTTAGCGGCAGCGGGAGT |
| SEQ ID | GGAACGGACTTCACTCTCACGATTTCATCGTTGCAACCCGAGGACTTTGCAACATATTATTGCCAGC |
| NO: 1223 | AGAGCTATAGCACTCCATTCACCTTCGGGCCTGGGACAAAGGTCGATATCAAGGGAAGCACTTCAGG |
| | AAGTGGCAAACCTGGGTCTGGGGAGGGTTCGACCAAGGGACAGGTCCAACTGCAAGAATCAGGTCCC |
| | GGGCTGGTTAAACCTTCAGAAACCTTGTCCTTAACTTGTACCGTATCAGGCGGAAGTATCTCCTCCT |
| | CTTCTTATTATCGAGGCTGGATTAGACAGCCACCCGGCAAAGGCCTTGAGTGGATTGGCTCCATCTA |
| | CTACAGCGGCAGCACGTACTATAACCCAAGCCTGAAAAGTAGGGTGACTATCTCTGTGGACACTTCT |
| | AAGAATCAGTTCAGTCTCAAACTTTCATCCGTGACCGCCGCGGACACCGCTGTGTACTATTGTGCTA |
| | GACACCCGCGTGGAAGTTACGGCGCTAATTTTGATTACTGGGGTCAGGGAACACTCGTTACAGTGTC |
| | CTCC |
| SLC34A2 | CAAGTGCAACTGCAAGAATCAGGGCCTGGTTTGGTCAAGCCATCTGAGACACTTTCCTTAACTTGTA |
| Ab 12 scFv | CGGTATCTGGTGGGTCAATCTCCTCCTCATCATATTACCGCGGGTGGATTAGGCAGCCACCCGGCAA |
| (VH-VL) | AGGACTGGAGTGGATTGGAAGTATATACTATTCCGGCTCAACGTACTACAACCCAAGCCTGAAGAGT |
| SEQ ID | CGGGTCACTATAAGTGTGGACACTAGCAAGAATCAATTTTCCCTGAAACTCTCGAGCGTTACCGCAG |
| NO: 1224 | CCGATACCGCGGTGTACTATTGTGCTAGACACCCTCGAGGGAGCTATGGCGCTAATTTCGACTATTG |
| | GGGTCAGGGAACATTGGTTACTGTGTCCTCCGGCAGCACCAGCGGGTCAGGGAAACCTGGGAGCGGC |
| | GAAGGCTCTACCAAGGGCGAAATTGTACTCACACAGAGTCCATCTTCCCTTTCCGCAAGTGTCGGAG |
| | ACAGAGTTACTATTACTTGCAGGGCCTCTCAGAGCATCTCTAGTTACCTGAACTGGTACCAGCAGAA |
| | ACCGGGCAAAGCACCCAAGCTCCTAATCTACGCTGCCAGCTCTCTGCAGTCTGGTGTGCCTAGTCGT |
| | TTTTCAGGAAGCGGCTCCGGAACAGATTTCACCCTCACCATCAGCTCGTTACAGCCGGAGGACTTCG |
| | CCACCTATTACTGCCAGCAGAGCTATTCTACACCCTTCACCTTTGGACCCGGCACAAAAGTGGATAT |
| | CAAG |
| SLC34A2 | GACATTCAGATGACCCAATCTCCTTCTTCTCTGAGTGCTTCGGTTGGGGATCGGGTCACTGTCACCT |
| Ab 13 scFv | GTCGCGCTAACCAGGACATTAACAATTACCTGGCTTGGTACCAGCAGACACCCGGCAAAGTACCAAA |
| (VL-VH) | ACTCCTTATTTACGCCGCAAGCACTCTGCAGAGCGGAGTACCAAGTCGATTCAGCGGATCAGGCTCT |
| SEQ ID | GGGACTGATTTCACACTGACCATCTCCTCACTGCAACCTGAGGACGTCGCCACGTACTATTGCCAGA |
| NO: 1225 | ATTATTACAGCGTGCCCTGGACCTTTGGTCAGGGAACAAAGCTGGAAATCAAGGGCTCAACTTCTGG |
| | CTCAGGAAAACCGGGCAGCGGTGAAGGCTCCACCAAGGGACAGGTTCAGTTAGTTGAGTCTGGTGGC |
| | GGGCTTGTCCAACCCGGTAGGAGCTTGCGGCTGAGTTGCATAGGGAGTGGGTTCACGTTTGGGGAAT |
| | ATGCCATGAGTTGGGTGAGGCAGGCACCTGGCAAAGGGCTAGAGTGGGTGGGGTTCATCAGAACCAA |
| | GCCATACGGTGGCACCACTGAATTTGCGGCAAGCGTGAAGGGAAGATTCACGATGTCCCGCGATGAC |
| | TCCAAATCAATCGCCTATCTCGAGATGAACTCCCTCAAGACCGAGGACACTGCGGTGTACTATTGTA |
| | CACTCATACCCGCCCTTCGTTTTCTGGAGTGGTTGCCCTGGGGACAAGGCACATTGGTGACAGTGTC |
| | CTCC |
| SLC34A2 | GAGATCGTTCTGACCCAGTCACCTGATTTCCAGAGCGTAACACCTAAAGAATCTGTGACTATAACTT |
| Ab 14 scFv | GTCGGGCAAGCCAGTCTGTCGGATCAGGCCTCCACTGGTACCAGCAGAAGCCCGACCAGAGTCCCAA |
| (VL-VH) | GCTCCTAATCAAATATGCCAGTCAGAGCTTCTCAGGAGTCCCAAGTCGATTTTCAGGGAGTGGCAGC |
| SEQ ID | GGAACAGATTTCACATTGACAATCAATTCCCTGGAAGCCGAGGACGCGGCCACATATTATTGCCTCC |
| NO: 1226 | AATCTAGCAGCCTGCCCTGGACTTTTGGGCAAGGCACGAAGGTGGAAATCAAGGGCTCGACAAGCGG |
| | CTCTGGCAAACCGGGCTCTGGTGAAGGGTCTACCAAGGGACAAGTGCAACTGCAAGAGAGCGGACCA |
| | GGGCTGGTGAAACCCTCTGAGACCCTTTCCCTGACCTGTACGGTTTCAGGAGGGTCCGTGTCCTCCG |
| | GTAGTTATTACTGGTCATGGATTAGACAGCCTCCGGGTAAAGGGTTAGAGTGGATTGGCTACATATT |
| | CTACTCCGGGTCTACGTACTATAACCCTAGTTTGAAGTCGCGCGTTACCATTAGCGTGGACACTAGC |
| | AAGAACCAATTCTCCCTTAAGCTCACCAGTGTGACAGCCGCAGACACCGCTGTCTACTTTTGCGCTA |
| | GGTGGATGACCACTGTCAAAGGCTACTTTGATTATTGGGGTCAGGGAACCCTGGTAACTGTCTCCTC |
| | C |
| SLC34A2 | GATATTCAGATGACCCAAAGCCCTAGTTCCCTGTCCGCAAGTGTTGGGGACCGGGTGACTATTACAT |
| Ab 15 scFv | GCCGAGCTTCTCAAAGCATCAGCTCATACCTGAACTGGTACCAGCAGAAACCCGGCAAGGCACCTAA |
| (VL-VH) | GTTCCTGATTAGCCCAGCCTCTAGCCTGCAGTCTGGCGTACCCAGTCGCTTTTCTGGGTCCGGCTCT |
| SEQ ID | GGCACGGATTTCACGCTAACAATCAGCTCGCTTCAGCCGGAGGACTTTGCCACTTATTATTGCCAGC |
| NO: 1227 | AATCATATAGCATACCTTGGACATTTGGCCAAGGGACAAAGGTGGAAATCAAGGGATCAACCTCTGG |
| | CTCTGGCAAACCTGGGAGCGGTGAGGGAAGTACCAAAGGACAGGTTCAGTTGCAGGAAAGCGGGCCC |
| | GGGCTGGTCAAACCATCAGAAACCTTGTCCTTAACCTGTACCGTAAGCGGCGGAAGTGTGTCCTCCG |
| | CTTCATATTATTGGTCGTGGATAAGGCAGCCACCCGGAAAAGGGCTCGAGTACATTGGATACATCTA |
| | CTATTCAGGATCTACTTACTACAACCCAAGCCTGAAGTCAAGAGTGACCATCTCCATTGATACCTCC |
| | AAGAATCAGTTCAGTCTTAATCTCAGGAGTGTCACTGCTGCCGACACAGCGGTGTACTATTGCGCCA |
| | GATACATCGTCGGTCGTCCCGGCTTCAACTGGTTTGACCCGTGGGGTCAGGGTACACTCGTGACTGT |
| | GTCCTCC |
| SLC34A2 | GATATTCAGATGACTCAGAGTCCCTCATCCCTTTCCGCTTCTGTCGGCGACCGCGTTACAATCACCT |
| Ab 16 scFv | GTAGGGCCTCGCAGACAATCAGTTCGTACCTGAACTGGTATCAACAGAAACCTGGGAAGGCTCCTAA |
| (VL-VH) | ACTCCTCATAAGTCCTGCCTCAAATTTACAGTCTGGGGTCCCAAGCAGATTTTCTGGATCAGGGTCT |
| SEQ ID | GGGACGGATTTCACCCTGACTATCAGCTCGCTGCAGCCGGAGGACTTTGCCACGTATTATTGCCAGC |
| NO: 1228 | AATCATACATCATTCCCTGGACCTTTGGCCAGGGAACAAAGGTCGAGATCAAGGGTAGCACTAGCGG |
| | CAGCGGAAAACCGGGCTCTGGCGAAGGGTCTACCAAGGGTCAAGTGCAGCTCCAAGAATCAGGGCCC |
| | GGGTTGGTTAAACCAAGTGAAACCCTTTCCCTGACTTGCACCGTTAGCGGAGATTCTGTGTCCTCCG |
| | TGTCCTATTACTGGAGCTGGATACGACAGCCACCTGGCAAGGGACTGGAGTGGATTGGCTACATCTG |
| | GTACAGCGGCAGCACCTATTACAACCCAAGTCTAAAGTCTCGGGTGACAATTTCCATTGACACTTCA |
| | AAGAATCAGTTCAGCCTGAAGTTAAGGAGTGTGACCGCAGCCGACACTGCGGTGTACTATTGTGCAA |
| | GATACATCGTAGGCCGTCCCGGTTTCAACTGGTTCGATCCCTGGGGTCAGGGAACATTGGTGACAGT |
| | ATCCTCC |
| SLC34A2 | CAGGTCCAACTGCAAGAATCGGGTCCAGGGCTGGTGAAACCCAGCGAAACCCTTAGTCTGACTTGTA |
| Ab 16 scFv | CCGTATCCGGAGACTCCGTGTCCTCCGTCAGTTATTATTGGAGCTGGATTCGGCAGCCACCTGGGAA |
| (VH-VL) | GGGCCTGGAGTGGATTGGCTACATCTGGTACTCTGGGTCAACTTACTATAACCCATCGCTCAAAAGC |
| SEQ ID | CGCGTAACGATAAGTATCGACACGAGCAAAAATCAATTCTCATTGAAGCTCAGGAGCGTGACAGCCG |
| NO: 1229 | CAGATACTGCGGTGTACTATTGTGCTAGATACATCGTGGGTAGGCCCGGCTTCAACTGGTTTGATCC |
| | CTGGGGACAAGGCACACTCGTTACCGTGTCCTCCGGCAGCACTTCAGGGTCTGGAAAACCGGGAAGC |
| | GGAGAGGGCTCTACCAAGGGCGATATTCAGATGACCCAGTCTCCAAGCTCCCTTTCCGCTTCTGTTG |
| | GGGATCGAGTGACAATTACATGCAGAGCCAGTCAGACCATCTCATCTTACCTGAACTGGTATCAGCA |
| | AAAGCCCGGGAAGGCACCGAAACTGCTGATTAGTCCCGCCAGTAATCTACAGTCAGGTGTCCCTAGC |
| | CGTTTCTCTGGAAGCGGTTCTGGCACGGACTTCACGTTGACAATCAGTTCCTTACAGCCTGAGGACT |
| | TTGCCACCTATTACTGCCAGCAGTCATACATAATCCCTTGGACCTTTGGCCAGGGAACAAAGGTCGA |
| | AATCAAG |
| SLC34A2 | CAGGTTCAGCTCGTCCAGTCTGGGGCAGAAGTAAAAAAGCCTGGGGCTAGTGTGAAGGTGTCCTGTA |
| Ab 17 | AGGCGTCCGGATATACATTCACCGGCTATAACATCCACTGGGTTCGGCAGGCACCAGGCCAGGGACT |
| (VL-VH) | AGAATGGATGGGCGCTATATACCCAGGAAATGGAGATACTTCTTATGCCCAAAAATTTCAAGGCCGC |
| SEQ ID | GTTACCATGACAGCAGATACCAGCACTTCTACCGTCTACATGGAGCTCAGTAGCCTTAGGAGTGAGG |
| NO: 1230 | ACACGGCTGTGTACTATTGCGCCAGAGGAGAAACTGCGCGAGCCACCTTTGCCTACTGGGGACAAGG |
| | CACGCTGGTGACAGTGTCCTCCGGCAGCACAAGCGGGTCTGGGAAACCTGGTTCTGGGGAGGGCTCA |
| | ACGAAGGGCGACATTCAGATGACTCAGTCACCCTCATCCCTGTCCGCCTCAGTCGGAGATAGAGTGA |
| | CCATTACCTGTAGGGCCTCGCAGGACATTGGGAACTTCCTGAATTGGTACCAACAGAAACCCGGCAA |
| | GGCACCAAAAGTGCTGATCTACTACACCAGCTCCCTGTACTCAGGCGTACCCAGTCGTTTTAGCGGT |
| | AGTGGTAGCGGGACTGACTATACATTGACCATCTCGTCTTTGCAACCTGAAGATTTCGCTACTTATT |
| | ACTGCCAGCAGTATAGCAAACTCCCGCTTACATTCGGTCAGGGTACAAAGTTAGAGATCAAG |
| SLC34A2 | CAAGTGCAGTTGGTACAGTCTGGGGCAGAGGTCAAAAAACCCGGTGCAAGTGTTAAAATGAGTTGCA |
| Ab 18 | AGGCCTCCGGCTACACCTTTACCGGCTATAACATCCATTGGGTTCGGCAGGCACCGGGCCAGGGCCT |
| (VL-VH) | GGAATGGATAGGAGCTATCTACCCAGGCAATGGCGATACATCGTATGCTCAGAAGTTCCAAGGGCGC |
| SEQ ID | GCTACCCTCACCGCGGACACTAGCACCTCTACAGTGTATATGGAACTGTCTAGCCTGAGGTCCGAGG |
| NO: 1231 | ACACAGCGGTGTACTATTGCGCCAGAGGAGAGACTGCCCGAGCCACTTTCGCCTACTGGGGTCAGGG |
| | TACACTCGTGACTGTGTCCTCCGGGTCTACGTCAGGGAGTGGCAAACCTGGGTCTGGGGAGGGAAGT |
| | ACGAAGGGTGATATTCAGATGACTCAGAGCCCAAGCTCACTGTCAGCTTCGGTCGGCGATAGAGTAA |
| | CTATTACATGTCGTGCCAGTCAAGACATTGGAAATTTCCTTAACTGGTATCAGCAAAAGCCTGGAAA |
| | GGCACCCAAAGTTCTAATCTACTACACAAGCTCCTTGTACTCCGGCGTCCCAAGCAGGTTTTCAGGC |
| | TCAGGTTCTGGAACAGACTATACCCTGACCATATCCAGCCTTCAGCCCGAGGATTTTGCCACCTACT |
| | ATTGTCAACAGTACAGCAAACTCCCTCTAACCTTCGGGCAGGGAACGAAGTTAGAAATCAAG |
| SLC34A2 | GACATTCAAATGACTCAGTCGCCCTCTTCACTGTCAGCTTCTGTTGGGGATCGTGTAACCATTACCT |
| Ab 19 | GTAGGGCATCCCAGGATATAGGAAACTTCCTGAATTGGTATCAGCAAAAGCCCGGCAAAGCTCCAAA |
| (VL-VH) | GCTGCTGATCTACTACACCAGCAGCCTTTATTCAGGTGTCCCAAGCAGATTCTCCGGCAGCGGGTCC |
| SEQ ID | GGCACAGACTTCACCCTGACCATCTCTAGCCTACAGCCCGAGGACTTTGCAACATATTATTGTCAAC |
| NO: 1232 | AGTACAGTAAATTGCCACTGACGTTTGGGCAAGGTACAAAGCTCGAGATCAAGGGCTCGACAAGCGG |
| | CTCAGGCAAACCGGGCTCTGGGGAAGGAAGTACCAAGGGTCAGGTCCAGCTCGTACAGAGTGGAGCC |
| | GAAGTCAAAAAGCCTGGCGCGAGTGTTAAAGTGTCCTGCAAGGCCTCTGGGTACACATTCACCGGTT |
| | ACAACATCCACTGGGTTCGACAGGCTCCTGGCCAGGGACTTGAGTGGATGGGAGCCATTTACCCTGG |
| | GAATGGGGACACTTCTTATGCACAGAAATTTCAGGGTAGGGTGACTATGACTAGAGATACGTCAACG |
| | TCCACTGTCTACATGGAGTTGAGTAGCTTACGGAGCGAAGATACCGCCGTGTACTATTGCGCTCGCG |
| | GAGAGACAGCCCGGGCCACTTTTGCATATTGGGGACAAGGCACACTCGTGACCGTGTCCTCC |

In some embodiments, the antibody or antigen-binding fragment that binds to SLC34A2 comprises an scFv encoded by a nucleic acid sequence at least 80%, at least 85%, at least 90%, at least 91%, at least 92%, at least 93%, at least 94%, at least 95%, at least 96%, at least 97%, at least 98%, at least 99%, at least 99.5% or 100% identical to the nucleic acid sequence set forth in SEQ ID NOs: 1116, 1117, 1118, 1209, 1210, 1211, 1212, 1213, 1214, 1216, 1217, 1218, 1219, 1220, 1221, 1222, 1223, 1224, 1225, 1226, 1227, 1228, 1229, 1230, 1231, or 1232, optionally wherein the V_{H} CDRs and the V_{L} CDRs are identical to those encoded by 1116, 1117, 1118, 1209, 1210, 1211, 1212, 1213, 1214, 1216, 1217, 1218, 1219, 1220, 1221, 1222, 1223, 1224, 1225, 1226, 1227, 1228, 1229, 1230, 1231, or 1232, respectively. In some embodiments, the antibody or antigen-binding fragment that binds to SLC34A2 comprises an scFv encoded by the nucleic acid sequence set forth in any one of SEQ ID NOS: 1116, 1117, 1118, 1209, 1210, 1211, 1212, 1213, 1214, 1216, 1217, 1218, 1219, 1220, 1221, 1222, 1223, 1224, 1225, 1226, 1227, 1228, 1229, 1230, 1231, or 1232.

### Synthetic Immune Receptors

In some aspects, the present disclosure provides a synthetic immune receptor. In some embodiments, the synthetic immune receptor comprises an extracellular domain comprising a TMPRSS4 antibody or antigen-binding fragment thereof provided herein.

### Chimeric Antigen Receptors

In some aspects, the synthetic immune receptor is a chimeric antigen receptor (CAR). The CAR may be a human CAR, comprising fully human sequences, e.g., natural human sequences. An exemplary CAR comprises, from N-terminus to C-terminus, an antigen-binding domain (e.g., an extracellular antigen binding domain); a transmembrane domain; an intracellular co-stimulatory domain; and an intracellular activation domain.

In some embodiments, the chimeric antigen receptor includes an extracellular portion comprising an antigen binding domain. The antigen recognition domain of a receptor such as a CAR can be linked to one or more intracellular signaling components, such as signaling components that mimic activation through an antigen receptor complex, such as a TCR complex, in the case of a CAR, and/or signal via another cell surface receptor. Thus, in some embodiments, the extracellular binding component (e.g., ligand-binding or antigen-binding domain) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

In various embodiments, a CAR comprises means for binding a TMPRSS4 protein, optionally binding a human TMPRSS4 protein in the region(s) of human TMPRSS4 bound by the TMPRSS4 antibody or antigen binding fragment (e.g., as described in the Examples below). In some embodiments, the means binds a TMPRSS4 protein. In some embodiments, the means binds a human TMPRSS4 protein. In some embodiments, the means is a TMPRSS4 antibody or antigen-binding fragment or equivalent thereof (e.g., a full length antibody or a F(ab′)₂ fragment, a Fab fragment, a single chain variable fragment (scFv), and a single domain antibody (sdAb), or a functional fragment thereof) means for binding a TMPRSS4 protein. In some embodiments, the means for binding TMPRSS4 includes the anti-TMPRSS4 antibodies and antigen-binding fragments or equivalents thereof described herein.

In some aspects, the chimeric antigen receptor includes an extracellular portion comprising a TMPRSS4 antigen binding domain described herein and an intracellular signaling domain. In some embodiments, the antigen binding domain (e.g., an antibody or antigen binding fragment thereof) is referred to as a “binder.” In some embodiments, the antigen-binding domain is selected from the group consisting of an antibody, a nanobody, a diabody, a triabody, or a minibody, a F(ab′)₂ fragment, a Fab fragment, a single chain variable fragment (scFv), and a single domain antibody (sdAb), or a functional fragment thereof. In some embodiments, the antigen-binding moiety comprises an scFv. The antigen-binding moiety can include naturally-occurring amino acid sequences or can be engineered, designed, or modified so as to provide desired and/or improved properties, e.g., increased binding affinity. In some embodiments, an antibody or fragment includes an scFv, a V_{H}, or a single-domain V_{H} antibody and the intracellular domain contains an ITAM. In some aspects, the intracellular signaling domain includes a signaling domain of a zeta chain of a CD3-zeta (CD3) chain. In some embodiments, the chimeric antigen receptor includes a transmembrane domain linking the extracellular domain and the intracellular signaling domain.

In some aspects, the transmembrane domain contains a transmembrane portion of CD8α or CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 4-1BB.

In some embodiments, the N-terminus or C-terminus of the CAR comprises a post-translation modification, such as a deletion or modification of one or more amino acids. For example, the first, second or third N-terminus or C-terminus amino acid can be modified or deleted in the CAR.

In some embodiments, the CAR comprises SEQ ID NO:1164 wherein the N-terminal amino acid, the two N-terminal amino acids or the three N-terminal amino acids are different from those in SEQ ID NO: 1164, e.g., due to one or more posttranscriptional modification. In some embodiments, the CAR comprises SEQ ID NO:1164 wherein the C-terminal amino acid, the two C-terminal amino acids or the three C-terminal amino acids are different from those in SEQ ID NO: 1164, e.g., due to one or more posttranscriptional modification. In some embodiments, the CAR comprises SEQ ID NO:1166 wherein the N-terminal amino acid, the two N-terminal amino acids or the three N-terminal amino acids are different from those in SEQ ID NO: 1166, e.g., due to one or more posttranscriptional modification. In some embodiments, the CAR comprises SEQ ID NO:1166 wherein the C-terminal amino acid, the two C-terminal amino acids or the three C-terminal amino acids are different from those in SEQ ID NO: 1166, e.g., due to one or more posttranscriptional modification.

### CAR Transmembrane Domain

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CDS, CD9, CD16, CD22, CD33, CD37, CD64, CD80, CD86, CD 134, CD137, and/or CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

In some embodiments, the transmembrane domain of the receptor, e.g., the CAR, is a transmembrane domain of human CD28 or variant thereof, e.g., a 27-amino acid transmembrane domain of a human CD28 (Accession No.: P10747.1).

In some embodiments, the CAR comprises a CD8α transmembrane domain. In some embodiments, the CD8α transmembrane domain comprises the sequence set forth in SEQ ID NO: 822.

### CAR Hinge

In some embodiments, the CAR further includes a spacer, which may be or include at least a portion of an immunoglobulin constant region or variant or modified version thereof, such as a hinge region, e.g., a CD8α hinge, a CD4 hinge, a CD28 hinge, an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include CD8α hinge, IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153 or international patent application publication number WO2014031687. In some embodiments, the CAR hinge comprises a CD8α hinge. In some embodiments, the CD8α hinge comprises the sequence set forth in SEQ ID NO: 821.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the receptor.

### CAR Intracellular Domain

In some embodiments, upon ligation of the CAR, the cytoplasmic domain or intracellular signaling domain of the receptor activates at least one of the normal effector functions or responses of the immune cell, e.g., T cell engineered to express the receptor. In some embodiments, the CAR comprises means for activating at least one of the normal effector functions or responses of the immune cell, e.g., T cell engineered to express the receptor. For example, in some contexts, the receptor induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability. In some embodiments, the means for at least one of the normal effector functions or responses of the immune cell comprises a CAR intracellular activation domain, e.g., an intracellular activation domain provided herein or an equivalent thereof. In some embodiments, the means for at least one of the normal effector functions or responses of the immune cell comprises a CAR intracellular activation domain and a CAR co-stimulatory domain, e.g., a co-stimulatory domain provided herein or an equivalent thereof.

In some aspects, the receptor includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3 zeta.

In some embodiments, the intracellular signaling domain comprises a human CD3 zeta stimulatory signaling domain or functional variant thereof, such as a 112 AA cytoplasmic domain of isoform 3 of human CD3.zeta. (Accession No.: P20963.2) or a CD3 zeta signaling domain as described in U.S. Pat. No. 7,446,190 or U.S. Pat. No. 8,911,993.

The receptor, e.g., the CAR, can include at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the extracellular domain is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor-gamma, CD8, CD4, CD25, or CD16. For example, in some aspects, the CAR includes a chimeric molecule between CD3-zeta or Fc receptor-gamma and CD8, CD4, CD25 or CD16. In some embodiments, the CAR comprises a CD35 activation domain comprising the sequence set forth in SEQ ID NO: 824.

In some embodiments, the intracellular domain comprises an intracellular costimulatory signaling domain of 4-1BB or functional variant or portion thereof, such as a 42-amino acid cytoplasmic domain of a human 4-1BB (Accession No. Q07011.1) or functional variant or portion thereof.

In some embodiments, the receptor encompasses one or more, e.g., two or more, costimulatory domains and an activation domain, e.g., primary activation domain, in the cytoplasmic portion. Exemplary receptors include intracellular components of CD3-zeta, CD28, and 4-1BB. In some embodiments, the chimeric antigen receptor contains an intracellular domain of a T cell costimulatory molecule. In some aspects, the T cell costimulatory molecule is 4-1BB.

In some embodiments, the receptor includes a signaling domain and/or transmembrane portion of a costimulatory receptor, such as CD28, 4-1BB, OX40, DAP10, and ICOS. In some aspects, the same receptor includes both the activating and costimulatory components. In some embodiments, the same receptor includes multiple costimulatory components.

In certain embodiments, the intracellular signaling domain comprises a CD8α transmembrane and signaling domain linked to a CD3 (e.g., CD3-zeta) intracellular domain. In some embodiments, the intracellular signaling domain comprises a 4-1BB (CD137, TNFRSF9) co-stimulatory domains, linked to a CD3 zeta intracellular domain. In some embodiments, the CAR comprises a 4-1BB co-stimulatory domain. In some embodiments, the 4-1BB co-stimulatory domain comprises the sequence as set forth in SEQ ID NO: 823.

In some embodiments, the CAR or other antigen receptor further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a nerve growth factor receptor (NGFR), or epidermal growth factor receptor (e.g., tEGFR). In some embodiments, the nucleic acid encoding the marker is operably linked to a polynucleotide encoding for a linker sequence, such as a cleavable linker sequence or a ribosomal skip sequence, e.g., T2A. See WO2014031687. In some embodiments, introduction of a construct encoding the CAR and EGFRt separated by a T2A ribosome switch can express two proteins from the same construct, such that the EGFRt can be used as a marker to detect cells expressing such construct. In some embodiments, a marker, and optionally a linker sequence, can be any as disclosed in published patent application No. WO2014031687. For example, the marker can be a truncated EGFR (tEGFR) that is, optionally, linked to a linker sequence, such as a T2A ribosomal skip sequence.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

The CAR may comprise one or more modified or synthetic amino acids in place of one or more naturally-occurring amino acids. Exemplary modified amino acids include, but are not limited to, aminocyclohexane carboxylic acid, norleucine, α-amino n-decanoic acid, homoserine, S-acetylaminomethylcysteine, trans-3- and trans-4-hydroxyproline, 4-aminophenylalanine, 4-nitrophenylalanine, 4-chlorophenylalanine, 4-carboxyphenylalanine, (3-phenylserine (3-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbomane)-carboxylic acid, α,γ-diaminobutyric acid, α,γ-diaminopropionic acid, homophenylalanine, and α-tertbutylglycine.

For example, in some embodiments, the CAR includes an antibody or fragment thereof, including single chain antibodies (sdAbs, e.g., containing only the V_{H} region), V_{H} domains, and scFvs, described herein, a spacer such as a CD8α hinge, a CD8α transmembrane domain, a 4-1BB intracellular signaling domain, and a CD3 zeta signaling domain. In some embodiments, the CAR includes an antibody or fragment, including sdAbs and scFvs described herein, a spacer such as a CD8α hinge, a CD8α transmembrane domain, a 4-1BB intracellular signaling domain, and a CD3 zeta signaling domain.

Exemplary sequences of CAR components are provided in Table 13.

TABLE 13
CAR Components

| Component | Sequence |
|---|---|
| CD8α Hinge Domain | TTTPAPRPPTPAPTIASQPLSLRPEACRPAAGGAVHTRGLDFACD (SEQ ID NO: |
| | 821) |
| CD8α Transmembrane | IYIWAPLAGTCGVLLLSLVITLYC (SEQ ID NO: 822) |
| Domain | |
| 4-1BB Co-stimulatory | KRGRKKLLYIFKQPFMRPVQTTQEEDGCSCRFPEEEEGGCEL (SEQ ID NO: 823) |
| Domain | |
| CD3ζ Activation | RVKFSRSADAPAYQQGQNQLYNELNLGRREEYDVLDKRRGRDPEMGGKPRRKNPQEG |
| Domain | LYNELQKDKMAEAYSEIGMKGERRRGKGHDGLYQGLSTATKDTYDALHMQALPPR |
| | (SEQ ID NO: 824) |
| CD8α signal sequence | MALPVTALLLPLALLLHAARP (SEQ ID NO: 825) |
| Myc Tag | EQKLISEEDL (SEQ ID NO: 826) |
| Flag Tag | DYKDDDDK (SEQ ID NO: 959) |

Exemplary chimeric antigen receptor (CAR) sequences (nucleic acid and amino acid) are provided in Table 14. In some embodiments, the CAR is encoded by a sequence with at least 80%, 85%, 90%, 91%, 92%, 93%, 94%, 95%, 96%, 97%, 98%, 99%, or 100% identity to SEQ ID NOs: 1163 or 1165. In some embodiments, the CAR comprises a sequence with at least 80%, 85%, 90%, 91%, 92%, 93%, 94%, 95%, 96%, 97%, 98%, 99%, or 100% identity to SEQ ID NOs: 1164 or 1166.

TABLE 14
Exemplary CAR sequences

| Name and | |
|---|---|
| SEQ ID NO | Sequence |
| TMPRSS4 | CAAGTGCAGCTGGTCCAGTCTGGGGCGGAAGTGAAAAAGCCCGGAGCTAGTGTAAAGGTGTCCT |
| Ab48 CAR | GTAAAGCCAGCGGCTACACCTTCACCGGTTATTACCTGCATTGGGTCCGGCAGGCTCCTGGCCA |
| SEQ ID NO: | GGGCCTGGAGTGGATGGGCTGGATTTCCGCATATAACGGAAACACAAATTACGCCCAGAACCTG |
| 1163 | CAAGGCCGCGTGACCATGACCAGGGACACAAGCACTAGCACTGTCTACATGGAGTTGTCTAGCT |
| | TGAGAAGCGAAGATACCGCTGTGTACTATTGCGCCCGACACTCTTACTCGGGCTCATACTCAAC |
| | GCTACCCTATTATGGGATGGATGTTTGGGGTCAAGGGACAACGGTCACAGTATCCTCTGGAGGC |
| | GGTGGCAGCGGAGGAGGCGGGTCTGGAGGTGGTGGATCAGACATTCAGATGACCCAGTCACCAA |
| | GTTCCTTATCCGCAAGCGTTGGGGATCGTGTTACAATTACTTGCAGGGCCTCGCAAGGGATCTC |
| | TAATTATCTCGCTTGGTACCAGCAGAAACCTGGGAAAGCACCCAAGCTGCTGATCTACACTGCA |
| | AGCACACTTTTTCCAGGAGTGCCGTCAAGATTCTCTGGGTCCGGGAGTGGCACTGACTTCACCC |
| | TTACCATCTCCTCCCTCCAGCCTGAGGACTTTGCCACATATTATTGTCAACAGAGTTACTCCAT |
| | ACCACTCACGTTTGGCGGCGGAACAAAaGTtGAAATCAAGGCGGCAGCAaccacgacgccagcg |
| | ccgcgaccaccaacaccggcgcccaccatcgcgtcgcagccactgtcactgcgcccagaagcgt |
| | gccggccagcggcggggggcgcagtgcaTacgagggggctggacttcgcctgtgatatctacat |
| | ctgggcgcccttggccgggacttgtggggtccttctcctgtcactggttatcaccctttactgc |
| | aaacggggcagaaagaaactcctgtatatattcaaacaaccatttatgagaccagtacaaacta |
| | ctcaagaagaggacggctgtagctgccgatttccagaagaagaagaaggaggatgtgaactgag |
| | agtgaagttcagcaggagcgcagacgcccccgcgtaccagcagggccagaaccagctctataac |
| | gagctcaatctaggacgaagagaggagtacgatgttttggacaagaggcgtggccgggaccctg |
| | agatggggggaaagccgagaaggaagaaccctcaggaaggcctgtacaatgaactgcagaaaga |
| | taagatggcggaggcctacagtgagattgggatgaaaggcgagcgccggaggggcaaggggcac |
| | gatggcctttaccagggtctcagtacagccaccaaggacacctacgacgcccttcacatgcagg |
| | ccctgccccctaggtaa |
| TMPRSS4 | QVQLVQSGAEVKKPGASVKVSCKASGYTFTGYYLHWVRQAPGQGLEWMGWISAYNGNTNYAQNL |
| Ab48 CAR | QGRVTMTRDTSTSTVYMELSSLRSEDTAVYYCARHSYSGSYSTLPYYGMDVWGQGTTVTVSSGG |
| SEQ ID NO: | GGSGGGGSGGGGSDIQMTQSPSSLSASVGDRVTITCRASQGISNYLAWYQQKPGKAPKLLIYTA |
| 1164 | STLFPGVPSRFSGSGSGTDFTLTISSLQPEDFATYYCQQSYSIPLTFGGGTKVEIKAAATTTPA |
| | PRPPTPAPTIASQPLSLRPEACRPAAGGAVHTRGLDFACDIYIWAPLAGTCGVLLLSLVITLYC |
| | KRGRKKLLYIFKQPFMRPVQTTQEEDGCSCRFPEEEEGGCELRVKFSRSADAPAYQQGQNQLYN |
| | ELNLGRREEYDVLDKRRGRDPEMGGKPRRKNPQEGLYNELQKDKMAEAYSEIGMKGERRRGKGH |
| | DGLYQGLSTATKDTYDALHMQALPPR |
| TMPRSS4 | CAGGTCCAGTTGGTACAGAGCGGCGCCGAAGTGAAAAAGCCTGGGGCGTCCGTCAAAGTGTCTT |
| Ab49 CAR | GCAAGGCCTCCGGCTATACATTCACCGGGTACTACATGCATTGGGTGCGGCAGGCACCTGGCCA |
| SEQ ID NO: | GGGTCTAGAATGGATGGGCCGGATCAATCCCAACTCCGGCGGCACAAACTATGCTCAGAAATTT |
| 1165 | CAAGGTCGCGTCACCATGACCCGTGACACAAGTACGAGCACCGTCTACATGGAGCTGTCCTCCC |
| | TCAGGAGCGAGGATACAGCCGTGTACTATTGTGCAAGGGAGCGCGCCGGCTATAGCAGCGGGCA |
| | GTTCGATTATTGGGGACAAGGGACTCTGGTAACTGTGTCCTCCGGAGGCGGAGGATCAGGCGGA |
| | GGAGGCTCAGGAGGTGGAGGTTCTGACATTCAGATGACTCAATCTCCCTCGTCACTGTCAGCTA |
| | GTGTTGGGGATAGAGTGACTATTACCTGCCGAGCCAGTCAGTCAATATCTAACTGGCTCGCATG |
| | GTACCAGCAGAAGCCAGGGAAGGCTCCCAAACTGCTGATCTACGCCGCGAGCACCCTTCAGAAT |
| | GGCGTGCCGTCTAGATTTAGCGGTTCTGGGTCTGGGACCGACTTTACACTTACTATCAGTAGTT |
| | TACAACCAGAGGACTTTGCTACTTATTACTGTCAACAGAGCTACACCTTCCCTATTACGTTCGG |
| | CCAGGGAACAAAAGTTGAAATCAAGGCGGCAGCAaccacgacgccagcgccgcgaccaccaaca |
| | ccggcgcccaccatcgcgtcgcagccactgtcactgcgcccagaagcgtgccggccagcggcgg |
| | ggggcgcagtgcaTacgagggggctggacttcgcctgtgatatctacatctgggcgcccttggc |
| | cgggacttgtggggtccttctcctgtcactggttatcaccctttactgcaaacggggcagaaag |
| | aaactcctgtatatattcaaacaaccatttatgagaccagtacaaactactcaagaagaggacg |
| | gctgtagctgccgatttccagaagaagaagaaggaggatgtgaactgagagtgaagttcagcag |
| | gagcgcagacgcccccgcgtaccagcagggccagaaccagctctataacgagctcaatctagga |
| | cgaagagaggagtacgatgttttggacaagaggcgtggccgggaccctgagatggggggaaagc |
| | cgagaaggaagaaccctcaggaaggcctgtacaatgaactgcagaaagataagatggcggaggc |
| | ctacagtgagattgggatgaaaggcgagcgccggaggggcaaggggcacgatggcctttaccag |
| | ggtctcagtacagccaccaaggacacctacgacgcccttcacatgcaggccctgccccctaggt |
| | aa |
| TMPRSS4 | QVQLVQSGAEVKKPGASVKVSCKASGYTFTGYYMHWVRQAPGQGLEWMGRINPNSGGTNYAQKF |
| Ab49 CAR | QGRVTMTRDTSTSTVYMELSSLRSEDTAVYYCARERAGYSSGQFDYWGQGTLVTVSSGGGGSGG |
| SEQ ID NO: | GGSGGGGSDIQMTQSPSSLSASVGDRVTITCRASQSISNWLAWYQQKPGKAPKLLIYAASTLQN |
| 1166 | GVPSRFSGSGSGTDFTLTISSLQPEDFATYYCQQSYTFPITFGQGTKVEIKAAATTTPAPRPPT |
| | PAPTIASQPLSLRPEACRPAAGGAVHTRGLDFACDIYIWAPLAGTCGVLLLSLVITLYCKRGRK |
| | KLLYIFKQPFMRPVQTTQEEDGCSCRFPEEEEGGCELRVKFSRSADAPAYQQGQNQLYNELNLG |
| | RREEYDVLDKRRGRDPEMGGKPRRKNPQEGLYNELQKDKMAEAYSEIGMKGERRRGKGHDGLYQ |
| | GLSTATKDTYDALHMQALPPR |

### Synthetic Transcriptional Modulators

In various embodiments, the synthetic immune receptor is a synthetic transcriptional modulator. In some embodiments, the synthetic transcriptional modulator is a priming receptor (primeR). A priming receptor can activate transcription of a selected gene or genes following binding to a target antigen.

The recombinant synthetic immune receptor may be a synthetic human transcriptional modulator, comprising fully human sequences, e.g., natural human sequences. In various embodiments, the synthetic transcriptional modulator comprises (a) an extracellular antigen-binding domain, (b) a transmembrane domain comprising one or more-ligand inducible proteolytic sites, and (c) an intracellular domain comprising a human or humanized transcriptional effector.

In various embodiments, a synthetic transcriptional modulator comprises means for binding an SCL34A2 protein, optionally binding a human SCL34A2 protein in the region(s) of human SCL34A2 bound by a SCL34A2 antibody or antigen binding fragment (e.g., an antibody or antigen binding fragment thereof as described in the Examples below). In some embodiments, the means binds an SCL34A2 protein. In some embodiments, the means binds a human SCL34A2 protein. In some embodiments, the means is an SCL34A2 antibody or antigen-binding fragment or equivalent thereof (e.g., a full length antibody or a F(ab′)₂ fragment, a Fab fragment, a single chain variable fragment (scFv), and a single domain antibody (sdAb), or a functional fragment thereof) means for binding an SCL34A2 protein. In some embodiments, the means for binding SCL34A2 includes the anti-SCL34A2 antibodies and antigen-binding fragments or equivalents thereof described herein.

In some aspects, the synthetic transcriptional modulator includes an extracellular portion comprising an SLC34A2 antigen binding domain described herein and an intracellular signaling domain. In some embodiments, the antigen binding domain (e.g., an antibody or antigen binding fragment thereof) is referred to as a “binder.” In various embodiments, the antigen-binding domain of the synthetic transcriptional modulator described herein is selected from the group consisting of an antibody, a nanobody, a diabody, a triabody, or a minibody, a F(ab′)₂ fragment, a Fab fragment, a single chain variable fragment (scFv), and a single domain antibody (sdAb), or a functional fragment thereof. In some embodiments, the antigen-binding moiety comprises an scFv. The antigen-binding moiety can include naturally-occurring amino acid sequences or can be engineered, designed, or modified so as to provide desired and/or improved properties, e.g., increased binding affinity. In some embodiments, an antibody or fragment includes an scFv, a VH, or a single-domain V_{H}H antibody. In some embodiments, the antigen binding domain is referred to as a “binder.”

In various embodiments, the synthetic transcriptional modulator is based on the Notch protein (i.e., a synNotch). Binding of a natural Notch receptor to a cognate ligand, such as those from the Delta family of proteins, causes intramembrane proteolysis that cleaves an intracellular fragment of the Notch protein. This intracellular fragment is a transcriptional regulator that only functions when cleaved from Notch. Cleavage may occur by sequential proteolysis by ADAM metalloprotease and the gamma-secretase complex. This intracellular fragment enters the nucleus of a cell and activates cell-cell signaling genes. In contrast to a natural Notch protein, a synNotch replaces the natural Notch intracellular fragment with one that causes a gene encoding a protein of choice, such as a CAR, to be transcribed upon release of the intracellular fragment from the synthetic transcriptional modulator.

Notch receptors have a modular domain organization. The ectodomains of Notch receptors consist of a series of N-terminal epidermal growth factor (EGF)-like repeats that are responsible for ligand binding. In synNotch receptors, the Notch ligand-binding domain is replaced with a ligand binding domain that binds a selected target ligand or antigen. The EGF repeats are followed by three LIN-12/Notch repeat (LNR) modules, which are unique to Notch receptors, and are widely reported to participate in preventing premature receptor activation. The heterodimerization (HD) domain of Notch1 is divided by furin cleavage, so that its N-terminal part terminates the extracellular subunit, and its C-terminal half constitutes the beginning of the transmembrane subunit. Following the extracellular region, the receptor has a transmembrane segment and an intracellular domain (ICD), which includes a transcriptional regulator.

Multiple forms of synthetic transcriptional modulators can be used in the methods, cells, and nucleic acids as described herein. One type of synthetic transcriptional modulator contemplated for use in the methods and cells herein comprise a heterologous extracellular ligand binding domain, a linking polypeptide having substantial sequence identity with a Notch receptor including the NRR, a TMD, and an ICD. “Fn Notch” receptors comprise a heterologous extracellular ligand binding domain, a linking polypeptide having substantial sequence identity with a Robo receptor (such as a mammalian Robo1, Robo2, Robo3, or Robo4), followed by 1, 2, or 3 fibronectin repeats (“Fn”), a TMD, and an ICD. “Mini Notch” receptors comprise a heterologous extracellular ligand binding domain, a linking polypeptide having substantial sequence identity with a Notch receptor (lacking the NRR), a TMD, and an ICD. “Minimal Linker Notch” receptors comprise a heterologous extracellular ligand binding domain, a linking polypeptide lacking substantial sequence identity with a Notch receptor (e.g., a synthetic (GGS) n polypeptide sequence), a TMD, and an ICD. “Hinge Notch” receptors comprise a heterologous extracellular ligand binding domain, a hinge sequence comprising an oligomerization domain (i.e., a domain that promotes dimerization, trimerization, or higher order multimerization with a synthetic receptor and/or an existing host receptor), a TMD, and an ICD. All of these receptor classes are synthetic, recombinant, and do not occur in nature. In some embodiments, the non-naturally occurring receptors disclosed herein bind a target cell-surface displayed ligand, which triggers proteolytic cleavage of the receptors and release of a transcriptional regulator that modulates a custom transcriptional program in the cell. In some embodiments, the synthetic transcriptional modulator does not include a LIN-12-Notch repeat (LNR) and/or a heterodimerization domain (HD) of a Notch receptor.

### Synthetic Transcriptional Modulator Transmembrane Domain

As described above, the synthetic transcriptional modulator comprises a transmembrane domain (TMD) comprising one or more ligand-inducible proteolytic cleavage sites. In some embodiments, the TMD comprises a Notch1 transmembrane domain. In some embodiments, the transmembrane domain comprises the sequence as set forth in SEQ ID NO: 828.

Generally, the TMD suitable for the chimeric receptors disclosed herein can be any transmembrane domain of a Type 1 transmembrane receptor including at least one gamma-secretase cleavage site. Detailed description of the structure and function of the gamma-secretase complex as well as its substrate proteins, including amyloid precursor protein (APP) and Notch, can, for example, be found in a recent review by Zhang et al, Frontiers Cell Neurosci (2014). Non limiting suitable TMDs from Type 1 transmembrane receptors include those from CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A, and IFNAR2, wherein the TMD includes at least one gamma secretase cleavage site. Additional TMDs suitable for the compositions and methods described herein include, but are not limited to, transmembrane domains from Type 1 transmembrane receptors ILIR1, IL1R2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, YASN, FLT1, CDH5, PKHD1, NECTIN1, PCDHGC3, NRG1, LRP1B, CDH2, NRG2, PTPRK, SCN2B, Nradd, and PTPRM. In some embodiments, the TMD of the chimeric polypeptides or Notch receptors of the disclosure is a TMD derived from the TMD of a member of the calsyntenin family, such as, alcadein alpha and alcadein gamma. In some embodiments, the TMD of the chimeric polypeptides or Notch receptors of the disclosure is a TMD known for Notch receptors. In some embodiments, the TMD of the chimeric polypeptides or Notch receptors of the disclosure is a TMD derived from a different Notch receptor. For example, in a Mini Notch based on human Notch1, the Notch1 TMD can be substituted with a Notch2 TMD, Notch3 TMD, Notch4 TMD, or a Notch TMD from a non-human animal such as Danio rerio, Drosophila melanogaster, Xenopus laevis, or Gallus gallus.

In some embodiments, the synthetic transcriptional modulator comprises a Notch cleavage site, such as S2 or S3. Additional proteolytic cleavage sites suitable for the compositions and methods disclosed herein include, but are not limited to, ADAM10, a metalloproteinase cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). Another example of a suitable protease cleavage site is a plasminogen activator cleavage site, e.g., a urokinase plasminogen activator (uPA) or a tissue plasminogen activator (tPA) cleavage site. Another example of a suitable protease cleavage site is a prolactin cleavage site. Specific examples of cleavage sequences of uPA and tPA include sequences comprising Val-Gly-Arg. Another example of a protease cleavage site that can be included in a proteolytically cleavable linker is a tobacco etch vims (TEV) protease cleavage site, e.g., Glu-Asn-Leu-Tyr-Thr-Gln-Ser (SEQ ID NO: 833), where the protease cleaves between the glutamine and the serine. Another example of a protease cleavage site that can be included in a proteolytically cleavable linker is an enterokinase cleavage site, e.g., Asp-Asp-Asp-Asp-Lys (SEQ ID NO: 834), where cleavage occurs after the lysine residue. Another example of a protease cleavage site that can be included in a proteolytically cleavable linker is a thrombin cleavage site, e.g., Leu-Val-Pro-Arg (SEQ ID NO: 835). Additional suitable linkers comprising protease cleavage sites include sequences cleavable by the following proteases: a PreScission™ protease (a fusion protein comprising human rhinovirus 3C protease and glutathione-S-transferase), a thrombin, cathepsin B, Epstein-Barr vims proteas, MMP-3 (stromelysin), MMP-7 (matrilysin), MMP-9; thermolysin-like MMP, matrix metalloproteinase 2 (MMP-2), cathepsin L; cathepsin D, matrix metalloproteinase 1 (MMP-1), urokinase-type plasminogen activator, membrane type 1 matrixmetalloprotemase (MT-MMP), stromelysin 3 (or MMP-11), thermo lysin, fibroblast collagenase and stromelysin-1, matrix metalloproteinase 13 (collagenase-3), tissue-type plasminogen activator (tPA), human prostate-specific antigen, kallikrein (hK3), neutrophil elastase, and calpain (calcium activated neutral protease). Proteases that are not native to the host cell in which the receptor is expressed (for example, TEV) can be used as a further regulatory mechanism, in which activation of the receptor is reduced until the protease is expressed or otherwise provided. Additionally, a protease may be tumor-associated or disease-associated (expressed to a significantly higher degree than in normal tissue), and serve as an independent regulatory mechanism. For example, some matrix metalloproteases are highly expressed in certain cancer types.

In some embodiments, the amino acid substitution(s) within the TMD includes one or more substitutions within a “GV” motif of the TMD. In some embodiments, at least one of such substitution(s) comprises a substitution to alanine. Additional sequences and substitutions are described in WO2021061872, hereby incorporated by reference in its entirety.

### Synthetic Transcriptional Modulator Intracellular Domain

In some embodiments, the synthetic transcriptional modulator comprises one or more intracellular domains from or derived from a transcriptional regulator and/or a DNA-binding domain. In some embodiments, the intracellular domain comprises means for modulating transcription of one or more genes. In some embodiments, the means for means for modulating transcription of one or more genes comprises a transcriptional regulator, e.g., a transcriptional regulator provided herein or an equivalent thereof. In some embodiments, the intracellular domain comprises an HNF1α/p65 domain or a Gal4/VP64 domain. In some embodiments, the intracellular domain comprises a human or humanized intracellular domain. In some embodiments, the intracellular domain comprises the sequence as set forth in SEQ ID NO: 832.

Transcriptional regulators either activate or repress transcription from cognate promoters. Transcriptional activators typically bind nearby to transcriptional promoters and recruit RNA polymerase to directly initiate transcription. Transcriptional repressors bind to transcriptional promoters and sterically hinder transcriptional initiation by RNA polymerase. Other transcriptional regulators serve as either an activator or a repressor depending on where it binds and cellular conditions. Accordingly, as used herein, a “transcriptional activation domain” refers to the domain of a transcription factor that interacts with transcriptional control elements and/or transcriptional regulatory proteins (i.e., transcription factors, RNA polymerases, etc.) to increase and/or activate transcription of one or more genes. Non-limiting examples of transcriptional activation domains include: a herpes simplex virus VP16 activation domain, VP64 (which is a tetrameric derivative of VP16), HIV TAT, a NFkB p65 activation domain, p53 activation domains 1 and 2, a CREB (cAMP response element binding protein) activation domain, an E2A activation domain, NFAT (nuclear factor of activated T-cells) activation domain, yeast Gal4, yeast GCN4, yeast HAP1, MLL, RTG3, GLN3, OAF1, PIP2, PDR1, PDR3, PHO4, LEU3 glucocorticoid receptor transcription activation domain, B-cell POU homeodomain protein Oct2, plant Ap2, or any others known to one or ordinary skill in the art. In some embodiments, the transcriptional regulator is selected from Gal4-VP16, Gal4-VP64, tetR-VP64, ZFHD1-YP64, Gal4-KRAB, and HAP1-VP16. In some embodiments, the transcriptional regulator is Gal4-VP64. A transcriptional activation domain can comprise a wild-type or naturally occurring sequence, or it can be a modified, mutant, or derivative version of the original transcriptional activation domain that has the desired ability to increase and/or activate transcription of one or more genes. In some embodiments, the transcriptional regulator can further include a nuclear localization signal.

In some embodiments, the synthetic transcriptional modulator comprises one or more intracellular “DNA-binding domains” (or “DB domains”). Such “DNA-binding domains” refer to sequence-specific DNA binding domains that bind a particular DNA sequence element. Accordingly, as used herein, a “sequence-specific DNA-binding domain” refers to a protein domain portion that has the ability to selectively bind DNA having a specific, predetermined sequence. A sequence-specific DNA binding domain can comprise a wild-type or naturally occurring sequence, or it can be a modified, mutant, or derivative version of the original domain that has the desired ability to bind to a desired sequence. In some embodiments, the sequence-specific DNA binding domain is engineered to bind a desired sequence. Non-limiting examples of proteins having sequence-specific DNA binding domains that can be used in synthetic proteins described herein include HNF1a, Gal4, GCN4, reverse tetracycline receptor, THY1, SYN1, NSE/RU5′, AGRP, CALB2, CAMK2A, CCK, CHAT, DLX6A, EMX1, zinc finger proteins or domains thereof, CRISPR/Cas proteins, such as Cas9, Cas3, Cas4, Cas5, Cas5e (or CasD), Cash, Cas6e, Casof, Cas7, Cas8a1, Cas8a2, Cas8b, Cas8c, Cas10, Cas10d, CasF, CasG, CasH, Csy1, Csy2, Csy3, Csel (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu196, and TALEN.

In those embodiments where a CRISPR/Cas-like protein is used, the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the functions of the systems described herein. For example, a CRISPR enzyme that is used as a DNA binding protein or domain thereof can be mutated with respect to a corresponding wild-type enzyme such that the mutated CRISPR or domain thereof lacks the ability to cleave a nucleic acid sequence containing a DNA binding domain target site. For example, a D10A mutation can be combined with one or more of H840A, N854A, or N863A mutations to produce a Cas9 enzyme substantially lacking all DNA cleavage activity.

### Synthetic Transcriptional Modulator Juxtamembrane Domain

The ECD and the TMD, or the TMD and the ICD, can be linked to each other with a linking polypeptide, such as a juxtamembrane domain. SynNotches comprise a heterologous extracellular ligand-binding domain, a linking polypeptide having substantial sequence identity with a Notch receptor JMD (including the NRR), a TMD, and an ICD. “Fn Notch” receptors comprise a heterologous extracellular ligand binding domain, a linking polypeptide having substantial sequence identity with a Robo receptor (such as a mammalian Robo1, Robo2, Robo3, or Robo4), followed by 1, 2, or 3 fibronectin repeats (“Fn”), a TMD, and an ICD. “Mini Notch” receptors comprise a heterologous extracellular ligand binding domain, a linking polypeptide having substantial sequence identity with a Notch receptor JMD but lacking the NRR (the LIN-12-Notch repeat (LNR) modules, and the heterodimerization domain), a TMD, and an ICD. “Minimal Linker Notch” receptors comprise a heterologous extracellular ligand-binding domain, a linking polypeptide lacking substantial sequence identity with a Notch receptor (for example, without limitation, having a synthetic (GGS) n polypeptide sequence), a TMD, and an ICD. “Hinge Notch” receptors comprise a heterologous extracellular ligand-binding domain, a hinge sequence comprising an oligomerization domain (i.e., a domain that promotes dimerization, trimerization, or higher order multimerization with a synthetic receptor and/or an existing host receptor), a TMD, and an ICD.

In some embodiments, the synthetic transcriptional modulator comprises a juxtamembrane domain (JMD) peptide in between the extracellular domain and the transmembrane domain. In some embodiments, the synthetic transcriptional modulator comprises a juxtamembrane domain (JMD) peptide in between the transmembrane domain and the intracellular domain. In some embodiments, the JMD peptide comprises an LWF motif. The use of LWF motifs in receptor constructs is described in U.S. Pat. No. 10,858,443, hereby incorporated by reference in its entirety. In some embodiments, the JMD peptide has substantial sequence identity to the JMD of Notch1, Notch2, Notch3, and/or Notch4. In some embodiments, the JMD peptide has substantial sequence identity to the Notch1, Notch2, Notch3, and/or Notch4 JMD, but does not include a LIN-12-Notch repeat (LNR) and/or a heterodimerization domain (HD) of a Notch receptor. In some embodiments, the JMD peptide does not have substantial sequence identity to the Notch1, Notch2, Notch3, and/or Notch4 JMD. In some embodiments, the JMD peptide includes an oligomerization domain which promotes formation of dimers, trimers, or higher order assemblages of the receptor. Such JMD peptides are described in WO2021061872, hereby incorporated by reference in its entirety.

In the Mini Notch receptor, the linking polypeptide is derived from a Notch JMD sequence after deletion of the NRR and HD domain. The Notch JMD sequence may be the sequence from Notch1, Notch2, Notch3, or Notch4, and can be derived from a non-human homolog, such as those from Drosophila, Gallus, Danio, and the like. Four to 50 amino acid residues of the remaining Notch sequence can be used as a polypeptide linker. In some embodiments, the length and amino acid composition of the linker polypeptide sequence are varied to alter the orientation and/or proximity of the ECD and the TMD relative to one another to achieve a desired activity of the chimeric polypeptide, such as the signal transduction level when ligand induced or in the absence of ligand.

In the Minimal Linker Notch receptor, the linking polypeptide does not have substantial sequence identity to a Notch JMD sequence, including the Notch JMD sequence from Notch1, Notch2, Notch3, or Notch4, or a non-human homolog thereof. Four to 50 amino acid residues can be used as a polypeptide linker. In some embodiments, the length and amino acid composition of the linker polypeptide sequence are varied to alter the orientation and/or proximity of the ECD and the TMD relative to one another to achieve a desired activity of the chimeric polypeptide of the disclosure. The Minimal Linker sequence can be designed to include or omit a protease cleavage site, and can include or omit a glycosylation site or sites for other types of post-translational modification. In some embodiments, the Minimal Linker does not comprise a protease cleavage site or a glycosylation site.

In some embodiments, the synthetic transcriptional modulator further comprises a hinge. Hinge linkers that can be used in the synthetic transcriptional modulator can include an oligomerization domain (e.g., a hinge domain) containing one or more polypeptide motifs that promote oligomer formation of the chimeric polypeptides via intermolecular disulfide bonding. In these instances, within the chimeric receptors disclosed herein, the hinge domain generally includes a flexible polypeptide connector region disposed between the ECD and the TMD. Thus, the hinge domain provides flexibility between the ECD and TMD and also provides sites for intermolecular disulfide bonding between two or more chimeric polypeptide monomers to form an oligomeric complex. In some embodiments, the hinge domain includes motifs that promote dimer formation of the chimeric polypeptides disclosed herein. In some embodiments, the hinge domain includes motifs that promote trimer formation of the chimeric polypeptides disclosed herein (e.g., a hinge domain derived from OX40). Hinge polypeptide sequences suitable for the compositions and methods of the disclosure can be naturally-occurring hinge polypeptide sequences (e.g., those from naturally-occurring immunoglobulins) or can be engineered, designed, or modified so as to provide desired and/or improved properties, e.g., modulating transcription. Suitable hinge polypeptide sequences include, but are not limited to, those derived from IgA, IgD, and IgG subclasses, such as IgG1 hinge domain, IgG2 hinge domain, IgG3 hinge domain, and IgG4 hinge domain, or a functional variant thereof. In some embodiments, the hinge polypeptide sequence contains one or more CXXC motifs. In some embodiments, the hinge polypeptide sequence contains one or more CPPC motifs (SEQ ID NO: 836).

Hinge polypeptide sequences can also be derived from a CD8α hinge domain, a CD28 hinge domain, a CD152 hinge domain, a PD-1 hinge domain, a CTLA4 hinge domain, an OX40 hinge domain, and functional variants thereof. In some embodiments, the hinge domain includes a hinge polypeptide sequence derived from a CD8 α hinge domain or a functional variant thereof. In some embodiments, the hinge domain includes a hinge polypeptide sequence derived from a CD28 hinge domain or a functional variant thereof. In some embodiments, the hinge domain includes a hinge polypeptide sequence derived from an OX40 hinge domain or a functional variant thereof. In some embodiments, the hinge domain includes a hinge polypeptide sequence derived from an IgG4 hinge domain or a functional variant thereof.

The Fn Notch linking polypeptide is derived from the Robo1 JMD, which contains a fibronectin repeat (Fn) domain, with a short polypeptide sequence between the Fn repeats and the TMD. The Fn Notch linking polypeptide does not contain a Notch negative regulatory region (NRR), or the Notch HD domain. The Fn linking polypeptide can contain 1, 2, 3, 4, or 5 Fn repeats. In some embodiments, the chimeric receptor comprises a Fn linking polypeptide having about 1 to about 5 Fn repeats, about 1 to about 3 Fn repeats, or about 2 to about 3 Fn repeats. The short polypeptide sequence between the Fn repeats and the TMD can be from about 2 to about 30 amino acid residues. In some embodiments, the short polypeptide sequence can be between about 5 and about 20 amino acids, of any sequence. In some embodiments, the short polypeptide sequence can be between about 5 and about 20 naturally-occurring amino acids, of any sequence. In some embodiments, the short polypeptide sequence can be between about 5 and about 20 amino acids, of any sequence but having no more than one proline. In some embodiments, the short polypeptide sequence can be between about 5 and about 20 amino acids, and about 50% or more of the amino acids are glycine. In some embodiments, the short polypeptide sequence can be between about 5 and about 20 amino acids, where the amino acids are selected from glycine, serine, threonine, and alanine. In some embodiments, the length and amino acid composition of the Fn linking polypeptide sequence can be varied to alter the orientation and/or proximity of the ECD and the TMD relative to one another to achieve a desired activity of the chimeric polypeptide of the disclosure.

### Synthetic Transcriptional Modulator Stop-Transfer Sequence

In some embodiments, the synthetic transcriptional modulator further comprises a stop-transfer sequence (STS) in between the transmembrane domain and the intracellular domains. The STS comprises a charged, lipophobic sequence. Without being bound by any theory, the STS serves as a membrane anchor, and is believed to prevent passage of the intracellular domain into the plasma membrane. The use of STS domains in synthetic transcriptional modulators is described in WO2021061872, hereby incorporated by reference in its entirety. Non-limiting exemplary STS sequences include APLP1, APLP2, APP, TGBR3, CSF1R, CXCL16, CX3CL1, DAG1, DCC, DNER, DSG2, CDH1, GHR, HLA-A, IFNAR2, IGF1R, IL1R1, ERN2, KCNE1, KCNE2, CHL1, LRP1, LRP2, LRP18, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, VASN, FLT1, CDH5, PKTFD1, NECTIN1, KL, IL6R, EFNB1, CD44, CLSTN1, LRP8, PCDHGC3, NRG1, LRP1B, JAG2, EFNB2, DLL1, CLSTN2, EPCAM, ErbB4, KCNE3, CDH2, NRG2, PTPRK, BTC, EPHA4, IL1R2, KCNE4, SCN2B, Nradd, PTPRM, Notch1, Notch2, Notch3, and Notch4 STS sequences. In some embodiments, the STS is heterologous to the transmembrane domain. In some embodiments, the STS is homologous to the transmembrane domain. STS sequences are described in WO2021061872, hereby incorporated by reference in its entirety.

In some embodiments, the stop-transfer-sequence comprises the sequence as set forth in SEQ ID NO: 829.

Exemplary sequences of synthetic transcriptional modulator components are provided in Table 15.

TABLE 15
Synthetic Transcriptional Modulator/Priming Receptor (PrimeR) Components

| Component | Sequence |
|---|---|
| primeR CD8 Hinge #2 | TTTPAPRPPTPAPTIASQPLSLRPEAC (SEQ ID NO: 827) |
| primeR transmembrane | FMYVAAAAFVLLFFVGCGVLLS (SEQ ID NO: 828) |
| domain | |
| primeR Stop-Transfer- | RKRRRQHGQLWFPEGFKVSEASKKKRREPLGEDSVGLKPLKNA (SEQ ID NO: 829) |
| Sequence (JMD) | |
| primeR HNF1α DNA- | MVSKLSQLQTELLAALLESGLSKEALLQALGEPGPYLLAGEGPLDKGESCGGGRGEL |
| Binding Domain | AELPNGLGETRGSEDETDDDGEDFTPPILKELENLSPEEAAHQKAVVETLLQEDPWR |
| | VAKMVKSYLQQHNIPQREVVDTTGLNQSHLSQHLNKGTPMKTQKRAALYTWYVRKQR |
| | EVAQQFTHAGQGGLIEEPTGDELPTKKGRRNRFKWGPASQQILFQAYERQKNPSKEE |
| | RETLVEECNRAECIQRGVSPSQAQGLGSNLVTEVRVYNWFANRRKEEAFRHKLAM |
| | (SEQ ID NO: 830) |
| primeR p65 Trans- | DEFPTMVFPSGQISQASALAPAPPQVLPQAPAPAPAPAMVSALAQAPAPVPVLAPGP |
| Activation Domain | PQAVAPPAPKPTQAGEGTLSEALLQLQFDDEDLGALLGNSTDPAVFTDLASVDNSEF |
| | QQLLNQGIPVAPHTTEPMLMEYPEAITRLVTGAQRPPDPAPAPLGAPGLPNGLLSGD |
| | EDFSSIADMDFSALLSQISS (SEQ ID NO: 831) |
| primeR HNF1a-p65 | MVSKLSQLQTELLAALLESGLSKEALLQALGEPGPYLLAGEGPLDKGESCGGGRGEL |
| Trans-Activation Domain | AELPNGLGETRGSEDETDDDGEDFTPPILKELENLSPEEAAHQKAVVETLLQEDPWR |
| | VAKMVKSYLQQHNIPQREVVDTTGLNQSHLSQHLNKGTPMKTQKRAALYTWYVRKQR |
| | EVAQQFTHAGQGGLIEEPTGDELPTKKGRRNRFKWGPASQQILFQAYERQKNPSKEE |
| | RETLVEECNRAECIQRGVSPSQAQGLGSNLVTEVRVYNWFANRRKEEAFRHKLAMTC |
| | RDEFPTMVFPSGQISQASALAPAPPQVLPQAPAPAPAPAMVSALAQAPAPVPVLAPG |
| | PPQAVAPPAPKPTQAGEGTLSEALLQLQFDDEDLGALLGNSTDPAVFTDLASVDNSE |
| | FQQLLNQGIPVAPHTTEPMLMEYPEAITRLVTGAQRPPDPAPAPLGAPGLPNGLLSG |
| | DEDESSIADMDFSALLSQISS (SEQ ID NO: 832) |

### Priming Receptors

As used herein, a “priming receptor” or “PrimeR” is a polypeptide comprising an extracellular antigen binding domain and a signaling component that relocates to the nucleus and activates an inducible promoter when the antigen binding domain binds its cognate antigen, e.g., a cognate antigen expressed on the surface of a cell.

In some aspects, a priming receptor comprises an extracellular antigen binding domain, a transmembrane domain comprising one or more ligand-inducible proteolytic cleavage sites; and an intracellular domain comprising a human or humanized transcriptional effector, wherein binding of the first antigen-binding domain to its cognate target results in cleavage at the one or more ligand-inducible proteolytic cleavage sites in the transmembrane domain. In various embodiments, the intracellular domain of the priming receptor is cleaved from the transmembrane domain upon binding of the priming receptor to the priming antigen. The intracellular domain is then capable of translocating into a cell nucleus where it induces expression of the chimeric antigen receptor.

Exemplary priming receptor (e.g., synthetic transcriptional modulators) sequences (nucleic acid and amino acid) are provided in Table 16. In some embodiments, the priming receptor is encoded by a sequence with at least 80%, 85%, 90%, 91%, 92%, 93%, 94%, 95%, 96%, 97%, 98%, 99%, or 100% identity to SEQ ID NOs: 1157, 1159, or 1161. In some embodiments, the priming receptor comprises a sequence with at least 80%, 85%, 90%, 91%, 92%, 93%, 94%, 95%, 96%, 97%, 98%, 99%, or 100% identity to SEQ ID NOs: 1158, 1160, or 1162.

TABLE 16
Exemplary Priming Receptors

| Name and | |
|---|---|
| SEQ ID NO | Sequence |
| SCL34A2 Ab | GAAATCGTCCTGACACAGTCTCCAGATTTTCAGAGCGTGACGCCAAAGGAGAAAGTGACAATTA |
| 1 (VL-VH) | CATGCCGGGCATCTCAGTCTGTTGGGTCTGGGTTGCATTGGTATCAGCAAAAGCCCGACCAGTC |
| primeR | ACCCAAACTGCTCATCAAATATGCAAGCCAGAGTTTTTCAGGCGTACCTTCACGATTTAGCGGA |
| SEQ ID NO: | AGTGGTTCTGGCACTGACTTCACCTTGACGATTAATAGCCTGGAAGTAGAAGACGCTGCCACTT |
| 1157 | TCTACTGCCTGCAAAGTAGCTCCCTGCCCTGGACTTTTGGGCAGGGTACTAAGGTCGAGATCAA |
| | GGGCTCGACAAGCGGAAGTGGCAAACCGGGCAGCGGCGAGGGAAGCACCAAGGGACAAGTGCAA |
| | CTGCAAGAGTCTGGACCCGGGCTGGTGAAACCAAGCGAGACATTATCCCTCACTTGTACCGTGT |
| | CAGGCGGTAGTGTGTCCTCCGGGAATTTCTACTGGAGTTGGATACGCCAGCCTCCTGGGAAGGG |
| | CCTTGAATGGATTGGCTACATCTACTATTCAGGCTCCACCTACTACAACCCGTCTTTGAAGTCA |
| | AGGGTTACGATAAGCGTCGATACCTCCAAAAACCAATTCTCCCTAAAGCTCAGATCGTTAACTG |
| | CCGCTGATACCGCGGTGTACTATTGTGCCCGTTGGATGACCAAAGTTAAGGGTTATTTCGACTA |
| | TTGGGGACAAGGGACACTTGTCACCGTGTCCTCCGCAACCAcgacgccagcgccgcgaccacca |
| | acaccggcgcccaccatcgcgtcgcagcccctgtccctgcgcccTgaggcgtgcttcatgtacg |
| | tggcggcggccgcctttgtgcttctgttcttcgtgggctgcggggtgctgctgtcccgtaaacg |
| | Cagacgtcaacacggtcaactgtggtttccagaaggttttaaggtctccgaagcaagtaagaag |
| | aaaagacgtgaaccactgggagaagatagcgtcggtctgaaaccactcaagaatgccatggttt |
| | ctaaactgagccagctgcagacggagctcctggcggccctgctggagtcagggctgagcaaaga |
| | ggcactgctccaggcactgggCgagccggggccctacctcctggctggagaaggccccctggac |
| | aagggggagtcctgcggcggcggtcgaggggagctggctgagctgcccaatgggctgggggaga |
| | ctcggggctccgaggacgagacCgacgacgatggggaagacttcacgccacccatcctcaaaga |
| | gctggagaacctcagccctgaggaggcggcccaccagaaagccgtggtggagacccttctgcag |
| | gaggacccgtggcgtgtggcgaagatggtcaagtcctacctgcagcagcacaacatcccacagc |
| | gggaggtggtcgataccactggcctcaaccagtcccacctgtcccaacacctcaacaagggcac |
| | tcccatgaagacgcagaagcgggccgccctgtacacctggtaTgtccgcaagcagcgagaggtg |
| | gcgcagcagttcacccatgcagggcagggagggctgattgaAgaGcccacaggAgatgagctac |
| | caaccaagaaggggcggaggaaccgtttcaagtggggcccagcatcccagcagatcctgttcca |
| | ggcctatgagaggcagaagaaccctagcaaggaggagcgagaAacgctagtggaggagtgcaat |
| | agggcggaatgcatccagagaggTgtgtcAccatcacaAgcacaAggTctgggctccaacctcg |
| | tcacggaggtgcgtgtctacaactggtttgccaaccggcgcaaagaagaagccttccggcacaa |
| | gctggccatgacctgcagggatgagtttcccaccatggtgtttccttctgggcagatcagccag |
| | gcctcggccttggccccggcccctccccaagtcctgccccaggctccagcccctgcccctgctc |
| | cagccatggtatcagctctggcccaggccccagcccctgtcccagtcctagccccaggccctcc |
| | tcaAgctgtggccccacctgcccccaagcccacccaAgctggggaaggaacgctgtcagaggcc |
| | ctgctgcagctgcagtttgatgatgaagacctgggggccttgcttggcaacagcacagacccag |
| | ctgtgttcacagacctggcatccgtcgacaactccgagtttcagcagctgctgaaccagggcat |
| | acctgtggccccccacacaactgagcccatgctgatggagtaccctgaggctataactcgccta |
| | gtgacaggggcccagaggccccccgacccagctcctgctccactgggggccccggggctcccca |
| | atggcctcctttcaggagatgaagacttctcctccattgcggacatggacttctcagccctgct |
| | gagtcagatcagctccTAA |
| SCL34A2 Ab | EIVLTQSPDFQSVTPKEKVTITCRASQSVGSGLHWYQQKPDQSPKLLIKYASQSFSGVPSRFSG |
| 1 (VL-VH) | SGSGTDFTLTINSLEVEDAATFYCLQSSSLPWTFGQGTKVEIKGSTSGSGKPGSGEGSTKGQVQ |
| primeR | LQESGPGLVKPSETLSLTCTVSGGSVSSGNFYWSWIRQPPGKGLEWIGYIYYSGSTYYNPSLKS |
| SEQ ID NO: | RVTISVDTSKNQFSLKLRSLTAADTAVYYCARWMTKVKGYFDYWGQGTLVTVSSATTTPAPRPP |
| 1158 | TPAPTIASQPLSLRPEACEMYVAAAAFVLLFFVGCGVLLSRKRRRQHGQLWFPEGFKVSEASKK |
| | KRREPLGEDSVGLKPLKNAMVSKLSQLQTELLAALLESGLSKEALLQALGEPGPYLLAGEGPLD |
| | KGESCGGGRGELAELPNGLGETRGSEDETDDDGEDFTPPILKELENLSPEEAAHQKAVVETLLQ |
| | EDPWRVAKMVKSYLQQHNIPQREVVDTTGLNQSHLSQHLNKGTPMKTQKRAALYTWYVRKQREV |
| | AQQFTHAGQGGLIEEPTGDELPTKKGRRNRFKWGPASQQILFQAYERQKNPSKEERETLVEECN |
| | RAECIQRGVSPSQAQGLGSNLVTEVRVYNWFANRRKEEAFRHKLAMTCRDEFPTMVFPSGQISQ |
| | ASALAPAPPQVLPQAPAPAPAPAMVSALAQAPAPVPVLAPGPPQAVAPPAPKPTQAGEGTLSEA |
| | LLQLQFDDEDLGALLGNSTDPAVFTDLASVDNSEFQQLLNQGIPVAPHTTEPMLMEYPEAITRL |
| | VTGAQRPPDPAPAPLGAPGLPNGLLSGDEDESSIADMDFSALLSQISS |
| SCL34A2 Ab | GAGATAGTCCTGACCCAGTCACCCGATTTCCAGAGTGTTACTCCTAAGGAGAAAGTGACTATAA |
| 2 (VL-VH) | CATGCCGGGCATCTCAGTCTGTCGGAAGCGGGCTACATTGGTACCAGCAGAAGCCTGACCAGAG |
| primeR | CCCGAAACTGCTCATCAAATATGCCTCCCAGTCGTTTTCTGGCGTGCCCTCTCGCTTTTCCGGA |
| SEQ ID NO: | AGCGGATCTGGCACAGACTTCACCTTGACCATCAATAGCCTGGAAACTGAGGACGCCGCTACGT |
| 1159 | ATTTCTGCCAGCAGTCCTCCAGTCTGCCTTGGACATTTGGTCAGGGAACGAAGGTGGAGATCAA |
| | GGGTTCAACATCAGGGAGCGGGAAACCGGGCTCTGGCGAGGGCTCAACAAAGGGACAAGTGCAA |
| | CTGCAAGAATCGGGACCCGGGCTGGTTAAACCAAGTGAAACCCTTTCCCTCACTTGTACCGTAA |
| | GCGGCGGTAGCGTGTCCTCTGGTAGCTACTATTGGAGTTGGATTAGGCAGGCGCCAGGGAAAGG |
| | CCTCGAATGGATTGGGTATATCTACTACAGCGGCAGTAACTACTACAACCCATCATTGAAGTCT |
| | AGAGTGACAATTAGTGTCGATACCTCTAAAAATCAATTCTCACTTAAGCTGCGAGCTGTAACCG |
| | CCGCAGACACTGCGGTGTACTATTGTGCCCGTTGGATGACCACTATCAAGGGCTACTTCGATTA |
| | TTGGGGACAAGGGACATTAGTTACGGTGTCCTCCGCAACCAcgacgccagcgccgcgaccacca |
| | acaccggcgcccaccatcgcgtcgcagcccctgtccctgcgcccTgaggcgtgcttcatgtacg |
| | tggcggcggccgcctttgtgcttctgttcttcgtgggctgcggggtgctgctgtcccgtaaacg |
| | Cagacgtcaacacggtcaactgtggtttccagaaggttttaaggtctccgaagcaagtaagaag |
| | aaaagacgtgaaccactgggagaagatagcgtcggtctgaaaccactcaagaatgccatggttt |
| | ctaaactgagccagctgcagacggagctcctggcggccctgctggagtcagggctgagcaaaga |
| | ggcactgctccaggcactgggCgagccggggccctacctcctggctggagaaggccccctggac |
| | aagggggagtcctgcggcggcggtcgaggggagctggctgagctgcccaatgggctgggggaga |
| | ctcggggctccgaggacgagacCgacgacgatggggaagacttcacgccacccatcctcaaaga |
| | gctggagaacctcagccctgaggaggcggcccaccagaaagccgtggtggagacccttctgcag |
| | gaggacccgtggcgtgtggcgaagatggtcaagtcctacctgcagcagcacaacatcccacagc |
| | gggaggtggtcgataccactggcctcaaccagtcccacctgtcccaacacctcaacaagggcac |
| | tcccatgaagacgcagaagcgggccgccctgtacacctggtaTgtccgcaagcagcgagaggtg |
| | gcgcagcagttcacccatgcagggcagggagggctgattgaAgaGcccacaggAgatgagctac |
| | caaccaagaaggggcggaggaaccgtttcaagtggggcccagcatcccagcagatcctgttcca |
| | ggcctatgagaggcagaagaaccctagcaaggaggagcgagaAacgctagtggaggagtgcaat |
| | agggcggaatgcatccagagaggTgtgtcAccatcacaAgcacaAggTctgggctccaacctcg |
| | tcacggaggtgcgtgtctacaactggtttgccaaccggcgcaaagaagaagccttccggcacaa |
| | gctggccatgacctgcagggatgagtttcccaccatggtgtttccttctgggcagatcagccag |
| | gcctcggccttggccccggcccctccccaagtcctgccccaggctccagcccctgcccctgctc |
| | cagccatggtatcagctctggcccaggccccagcccctgtcccagtcctagccccaggccctcc |
| | tcaAgctgtggccccacctgcccccaagcccacccaAgctggggaaggaacgctgtcagaggcc |
| | ctgctgcagctgcagtttgatgatgaagacctgggggccttgcttggcaacagcacagacccag |
| | ctgtgttcacagacctggcatccgtcgacaactccgagtttcagcagctgctgaaccagggcat |
| | acctgtggccccccacacaactgagcccatgctgatggagtaccctgaggctataactcgccta |
| | gtgacaggggcccagaggccccccgacccagctcctgctccactgggggccccggggctcccca |
| | atggcctcctttcaggagatgaagacttctcctccattgcggacatggacttctcagccctgct |
| | gagtcagatcagctccTAA |
| SCL34A2 Ab | EIVLTQSPDFQSVTPKEKVTITCRASQSVGSGLHWYQQKPDQSPKLLIKYASQSFSGVPSRFSG |
| 2 (VL-VH) | SGSGTDFTLTINSLETEDAATYFCQQSSSLPWTFGQGTKVEIKGSTSGSGKPGSGEGSTKGQVQ |
| primeR | LQESGPGLVKPSETLSLTCTVSGGSVSSGSYYWSWIRQAPGKGLEWIGYIYYSGSNYYNPSLKS |
| SEQ ID NO: | RVTISVDTSKNQFSLKLRAVTAADTAVYYCARWMTTIKGYFDYWGQGTLVTVSSATTTPAPRPP |
| 1160 | TPAPTIASQPLSLRPEACEMYVAAAAFVLLFFVGCGVLLSRKRRRQHGQLWFPEGFKVSEASKK |
| | KRREPLGEDSVGLKPLKNAMVSKLSQLQTELLAALLESGLSKEALLQALGEPGPYLLAGEGPLD |
| | KGESCGGGRGELAELPNGLGETRGSEDETDDDGEDFTPPILKELENLSPEEAAHQKAVVETLLQ |
| | EDPWRVAKMVKSYLQQHNIPQREVVDTTGLNQSHLSQHLNKGTPMKTQKRAALYTWYVRKQREV |
| | AQQFTHAGQGGLIEEPTGDELPTKKGRRNRFKWGPASQQILFQAYERQKNPSKEERETLVEECN |
| | RAECIQRGVSPSQAQGLGSNLVTEVRVYNWFANRRKEEAFRHKLAMTCRDEFPTMVFPSGQISQ |
| | ASALAPAPPQVLPQAPAPAPAPAMVSALAQAPAPVPVLAPGPPQAVAPPAPKPTQAGEGTLSEA |
| | LLQLQFDDEDLGALLGNSTDPAVFTDLASVDNSEFQQLLNQGIPVAPHTTEPMLMEYPEAITRL |
| | VTGAQRPPDPAPAPLGAPGLPNGLLSGDEDESSIADMDFSALLSQISS |
| SCL34A2 Ab | GATATTCAGATGACACAGAGCCCAAGTTCACTGTCAGCGAGTGTCGGAGATCGGGTTACTATAA |
| 3 (VL-VH) | CCTGCCGAGCATCTCAGGATATCAATAATTACCTGGCCTGGTATCAGCAAAAACCCGGCAAAGT |
| primeR | CCCGAAGCTCCTGATATACGCAGCTTCAACACTGCAAAGCGGTGTGCCCTCGCGCTTTAGCGGC |
| SEQ ID NO: | TCTGGCAGCGGGACAGATTTCACCCTGACGATTTCCTCCCTTCAACCCGAGGACGTGGCCACTT |
| 1161 | ACTACTCCCTCAACTATTACTCTGTACCCTGGACCTTTGGCCAAGGGACAAAGGTGGAAATCAA |
| | GGGCTCTACTAGCGGTTCAGGGAAACCTGGGAGTGGAGAGGGCTCGACCAAGGGACAGGTCCAG |
| | CTCGTGGAGTCTGGCGGTGGGCTGGTTCAGCCAGGGAGGAGTTTGCGGCTTTCCTGTACGGGAA |
| | GCGGATTCACCTTTGGTGACTATGCCATGAACTGGGTCAGGCAGGCTCCTGGAAAAGGCCTAGA |
| | GTGGGTGGGTTTCATCAGAACCAAGCCATATGGCGGCACTACAGAATATGCAGCCAGCGTAAAA |
| | GGGAGATTCACGTTCAGCCGCGACGACTCTAAGTCAATAGCTTACCTTCAGATGAACTCCCTCA |
| | AGACCGAAGACACCGCCGTGTACTATTGTACTATGATCCCTGTGCTGCGTTTTTTAGAGTGGTT |
| | GCCTTGGGGACAAGGGACATTAGTTACTGTGTCCTCCGCAACCAcgacgccagcgccgcgacca |
| | ccaacaccggcgcccaccatcgcgtcgcagcccctgtccctgcgcccTgaggcgtgcttcatgt |
| | acgtggcggcggccgcctttgtgcttctgttcttcgtgggctgcggggtgctgctgtcccgtaa |
| | acgCagacgtcaacacggtcaactgtggtttccagaaggttttaaggtctccgaagcaagtaag |
| | aagaaaagacgtgaaccactgggagaagatagcgtcggtctgaaaccactcaagaatgccatgg |
| | tttctaaactgagccagctgcagacggagctcctggcggccctgctggagtcagggctgagcaa |
| | agaggcactgctccaggcactgggCgagccggggccctacctcctggctggagaaggccccctg |
| | gacaagggggagtcctgcggcggcggtcgaggggagctggctgagctgcccaatgggctggggg |
| | agactcggggctccgaggacgagacCgacgacgatggggaagacttcacgccacccatcctcaa |
| | agagctggagaacctcagccctgaggaggcggcccaccagaaagccgtggtggagacccttctg |
| | caggaggacccgtggcgtgtggcgaagatggtcaagtcctacctgcagcagcacaacatcccac |
| | agcgggaggtggtcgataccactggcctcaaccagtcccacctgtcccaacacctcaacaaggg |
| | cactcccatgaagacgcagaagcgggccgccctgtacacctggtaTgtccgcaagcagcgagag |
| | gtggcgcagcagttcacccatgcagggcagggagggctgattgaAgaGcccacaggAgatgagc |
| | taccaaccaagaaggggcggaggaaccgtttcaagtggggcccagcatcccagcagatcctgtt |
| | ccaggcctatgagaggcagaagaaccctagcaaggaggagcgagaAacgctagtggaggagtgc |
| | aatagggcggaatgcatccagagaggTgtgtcAccatcacaAgcacaAggTctgggctccaacc |
| | tcgtcacggaggtgcgtgtctacaactggtttgccaaccggcgcaaagaagaagccttccggca |
| | caagctggccatgacctgcagggatgagtttcccaccatggtgtttccttctgggcagatcagc |
| | caggcctcggccttggccccggcccctccccaagtcctgccccaggctccagcccctgcccctg |
| | ctccagccatggtatcagctctggcccaggccccagcccctgtcccagtcctagccccaggccc |
| | tcctcaAgctgtggccccacctgcccccaagcccacccaAgctggggaaggaacgctgtcagag |
| | gccctgctgcagctgcagtttgatgatgaagacctgggggccttgcttggcaacagcacagacc |
| | cagctgtgttcacagacctggcatccgtcgacaactccgagtttcagcagctgctgaaccaggg |
| | catacctgtggccccccacacaactgagcccatgctgatggagtaccctgaggctataactcgc |
| | ctagtgacaggggcccagaggccccccgacccagctcctgctccactgggggccccggggctcc |
| | ccaatggcctcctttcaggagatgaagacttctcctccattgcggacatggacttctcagccct |
| | gctgagtcagatcagctccTAA |
| SCL34A2 Ab | DIQMTQSPSSLSASVGDRVTITCRASQDINNYLAWYQQKPGKVPKLLIYAASTLQSGVPSRFSG |
| 3 (VL-VH) | SGSGTDFTLTISSLQPEDVATYYSLNYYSVPWTFGQGTKVEIKGSTSGSGKPGSGEGSTKGQVQ |
| primeR | LVESGGGLVQPGRSLRLSCTGSGFTFGDYAMNWVRQAPGKGLEWVGFIRTKPYGGTTEYAASVK |
| SEQ ID NO: | GRFTFSRDDSKSIAYLQMNSLKTEDTAVYYCTMIPVLRFLEWLPWGQGTLVTVSSATTTPAPRP |
| 1162 | PTPAPTIASQPLSLRPEACEMYVAAAAFVLLFFVGCGVLLSRKRRRQHGQLWFPEGFKVSEASK |
| | KKRREPLGEDSVGLKPLKNAMVSKLSQLQTELLAALLESGLSKEALLQALGEPGPYLLAGEGPL |
| | DKGESCGGGRGELAELPNGLGETRGSEDETDDDGEDFTPPILKELENLSPEEAAHQKAVVETLL |
| | QEDPWRVAKMVKSYLQQHNIPQREVVDTTGLNQSHLSQHLNKGTPMKTQKRAALYTWYVRKQRE |
| | VAQQFTHAGQGGLIEEPTGDELPTKKGRRNRFKWGPASQQILFQAYERQKNPSKEERETLVEEC |
| | NRAECIQRGVSPSQAQGLGSNLVTEVRVYNWFANRRKEEAFRHKLAMTCRDEFPTMVFPSGQIS |
| | QASALAPAPPQVLPQAPAPAPAPAMVSALAQAPAPVPVLAPGPPQAVAPPAPKPTQAGEGTLSE |
| | ALLQLQFDDEDLGALLGNSTDPAVFTDLASVDNSEFQQLLNQGIPVAPHTTEPMLMEYPEAITR |
| | LVTGAQRPPDPAPAPLGAPGLPNGLLSGDEDFSSIADMDFSALLSQISS |

In some embodiments, the N-terminus or C-terminus of the synthetic transcriptional modulator, such as a priming receptor, comprises a post-translation modification, such as a deletion or modification of the amino acids. For example, the first, second or third N-terminus or C-terminus amino acid can be modified or deleted in the synthetic transcriptional modulator, such as a priming receptor.

In some embodiments, the synthetic transcriptional modulator, such as a priming receptor, comprises SEQ ID NO:1158 wherein the N-terminal amino acid, the two N-terminal amino acids or the three N-terminal amino acids are different from those in SEQ ID NO: 1158, e.g., due to one or more posttranscriptional modification. In some embodiments, the synthetic transcriptional modulator, such as a priming receptor, comprises SEQ ID NO: 1158 wherein the C-terminal amino acid, the two C-terminal amino acids or the three C-terminal amino acids are different from those in SEQ ID NO: 1158, e.g., due to one or more posttranscriptional modification. In some embodiments, the synthetic transcriptional modulator, such as a priming receptor, comprises SEQ ID NO: 1160 wherein the N-terminal amino acid, the two N-terminal amino acids or the three N-terminal amino acids are different from those in SEQ ID NO: 1160, e.g., due to one or more posttranscriptional modification. In some embodiments, the synthetic transcriptional modulator, such as a priming receptor, comprises SEQ ID NO: 1160 wherein the C-terminal amino acid, the two C-terminal amino acids or the three C-terminal amino acids are different from those in SEQ ID NO: 1160, e.g., due to one or more posttranscriptional modification. In some embodiments, the synthetic transcriptional modulator, such as a priming receptor, comprises SEQ ID NO: 1162 wherein the N-terminal amino acid, the two N-terminal amino acids or the three N-terminal amino acids are different from those in SEQ ID NO: 1162, e.g., due to one or more posttranscriptional modification. In some embodiments, the synthetic transcriptional modulator, such as a priming receptor, comprises SEQ ID NO: 1162 wherein the C-terminal amino acid, the two C-terminal amino acids or the three C-terminal amino acids are different from those in SEQ ID NO: 1162, e.g., due to one or more posttranscriptional modification.

### Logic Gate Systems

Provided herein are systems, e.g., logic gates, that induce killing, or cytolysis, of target cells and/or tissues expressing both TMPRSS4 and SLC34A2. In some embodiments, the target cells are in tumors or cancers. In some embodiments, the tissues are tumors or cancers. It has been discovered that making a T cell dependent on expression of both of these antigens improves tumor cell and/or tissue targeting specificity. Accordingly, provided herein are cells, e.g., immune cells such as T cells that are modified to target and kill cells and/or tissues that express TMPRSS4 or both TMPRSS4 and SLC34A2. In some embodiments, the target cell is a cancer cell. In some embodiments, the target tissues are tumors or cancers. In some embodiments a cell, e.g., an immune cell such as a T cell, is engineered to express a logic gate, e.g., an IF_THEN logic gate or an AND logic gate, comprising cell surface receptors to TMPRSS4 and SLC34A2. Exemplary engineered cells, e.g., T cells, comprise a first receptor that binds specifically to TMPRSS4 on the surface of a target cell, e.g., a cancer cell, which, when bound to TMPRSS4, induces the expression of a second receptor that binds specifically to SLC34A2 on the surface of the target cell, which second receptor triggers the killing or cytolysis of the target cell. Exemplary engineered cells, e.g., T cells, comprise a first receptor that binds specifically to SLC34A2 on the surface of a target cell, e.g., a cancer cell, which, when bound to SLC34A2, induces the expression of a second receptor that binds specifically to TMPRSS4 on the surface of the target cell, which second receptor triggers the killing or cytolysis of the target cell. The second receptor can be a chimeric antigen receptor.

In various embodiments, provided herein are polypeptide systems comprising a priming receptor (primeR) that binds to a first target antigen and a chimeric antigen receptor that binds to a second antigen. In some embodiments, the CAR antigen is TMPRSS4 and the priming receptor antigen is not TMPRSS4. In some embodiments, the CAR antigen is TMPRSS4 and the priming receptor antigen is SLC34A2. Such systems are alternatively termed “logic gates” or “circuits.”

As used herein, a “logic gate,” “circuit,” “circuit receptor,” “system” or “system receptor” refers to a two part or more polypeptide or polypeptide expression system comprising, e.g., a synthetic transcriptional activator such as a priming receptor, and a CAR, wherein one or more of the polypeptide(s) is dependent on the activity of another of the polypeptide(s) for activity or expression. In some embodiments, the polypeptide system comprises at least a first polypeptide comprising a synthetic transcriptional activator such as a priming receptor, and at least a second polypeptide comprising a CAR. The polypeptide expression system can be encoded on at least one nucleic acid inserted into a cell, where the synthetic transcriptional activator such as a priming receptor and the CAR are expressed in the cell. One or more suppressors of gene expression (e.g., an sgRNA or an shRNA) can also be employed to enhance expansion and activity of logic gate-expressing T cells (LG T cells or integrated circuit T (ICT) cells).

In various embodiments, the system comprises 4 steps leading to T cell activation: (1) the priming receptor (primeR) is constitutively expressed; (2) the priming receptor is triggered, resulting in cleavage of the intracellular domain; (3) the cleaved priming receptor intracellular domain induces expression of the CAR; and (4) the CAR is activated, resulting in T cell activation.

In some aspects, the system is encoded by nucleic acid transgenes inserted into an immune cell. The system can be encoded on a single nucleic acid insert or fragment that comprises both transgenes, or can be encoded on two nucleic acids that encode the system transgenes individually. The priming receptor and CAR of the system can be placed in any order on the single nucleic acid. For example, the priming receptor can be at the 5′ end and the CAR can be at the 3′ end or the CAR can be at the 5′ end and the primeR can be at the 3′ end.

A constitutive promoter can be operably linked to the nucleotide sequence encoding the priming receptor. An inducible promoter can also be operably linked to the nucleotide sequence encoding the CAR. In some embodiments, when the system is encoded on a single nucleic acid insert or fragment that comprises both transgenes, the nucleic acid can comprise, in a 5′ to 3′ direction, the constitutive promoter; the nucleotide sequence encoding the priming receptor; the inducible promoter; and the nucleotide sequence encoding the CAR. Alternatively, the nucleic acid can comprise, in a 5′ to 3′ direction, the inducible promoter; the nucleotide sequence encoding CAR; the constitutive promoter; and the nucleotide sequence encoding priming receptor. The one or more suppressors of gene expression, if present, can be present upstream or downstream of the primeR and/or the CAR.

Non-limiting examples of suitable promoters include constitutive and inducible promoters, such as EF1α or inducible Hepatocyte Nuclear Factor 1α (HNF1α)-YB TATA or RNA polymerase II (pol II)-based promoters. In some embodiments, the constitutive promoter is EF1α. In some embodiments, the EF1α promoter comprises as sequence as set forth in SEQ ID NO: 991. Non-limiting examples of suitable promoters further include the tetracycline inducible or repressible promoter, RNA polymerase I or III-based promoters, the pol II dependent viral promoters, such as the CMV-IE promoter, and the pol III U6 and H1 promoters, as well as Hepatocyte Nuclear Factor 1α (HNF1α)-YB TATA promotor provided in SEQ ID NO: 992. Table 17 provides the sequences of exemplary promoters.

TABLE 17
Exemplary Promoters

| Name | Sequence |
|---|---|
| EF1α promoter | GGATCTGCGATCGCTCCGGTGCCCGTCAGTGGGCAGAGCGCACATCGCCCACAG |
| (SEQ ID NO: | TCCCCGAGAAGTTGGGGGGAGGGGTCGGCAATTGAACGGGTGCCTAGAGAAGGT |
| 991) | GGCGCGGGGTAAACTGGGAAAGTGATGTCGTGTACTGGCTCCGCCTTTTTCCCG |
| | AGGGTGGGGGAGAACCGTATATAAGTGCAGTAGTCGCCGTGAACGTTCTTTTTC |
| | GCAACGGGTTTGCCGCCAGAACACAGCTGAAGCTTCGAGGGGCTCGCATCTCTC |
| | CTTCACGCGCCCGCCGCCCTACCTGAGGCCGCCATCCACGCCGGTTGAGTCGCG |
| | TTCTGCCGCCTCCCGCCTGTGGTGCCTCCTGAACTGCGTCCGCCGTCTAG |
| HNF1α response | gttaataattaacatatatgttaatcattaacatatagttaattattaaccgct |
| elements | atgttaatgattaacaacggttaataattaacatatatgttaatcattaacata |
| (SEQ ID NO: | ta |
| 1245) | |
| YB TATA | tctagagggtatataatgggggcca |
| promoter (SEQ ID | |
| NO: 1246) | |
| HNF1α-YB | gttaataattaacatatatgttaatcattaacatatagttaattattaaccgct |
| TATA inducible | atgttaatgattaacaacggttaataattaacatatatgttaatcattaacata |
| promoter | taactagtctagagggtatataatgggggcca |
| (SEQ ID NO: | |
| 992) | |

In some aspects, the system is encoded by a nucleic acid comprising a sequence with at least 80%, 85%, 90%, 91%, 92%, 93%, 94%, 95%, 96%, 97%, 98%, 99%, or 100% identity to a sequence comprising nucleotides 481-7257 of SEQ ID NO: 1120; a sequence with at least 80%, 85%, 90%, 91%, 92%, 93%, 94%, 95%, 96%, 97%, 98%, 99%, or 100% identity to a sequence comprising nucleotides 481-7239 of SEQ ID NO: 1121; a sequence with at least 80%, 85%, 90%, 91%, 92%, 93%, 94%, 95%, 96%, 97%, 98%, 99%, or 100% identity to a sequence comprising nucleotides 481-7621 of SEQ ID NO: 1122; a sequence with at least 80%, 85%, 90%, 91%, 92%, 93%, 94%, 95%, 96%, 97%, 98%, 99%, or 100% identity to a sequence comprising nucleotides 481-7636 of SEQ ID NO: 1123; or a sequence with at least 80%, 85%, 90%, 91%, 92%, 93%, 94%, 95%, 96%, 97%, 98%, 99%, or 100% identity to a sequence comprising nucleotides 481-7621 of SEQ ID NO: 1124. In some aspects, the system is encoded by a nucleic acid comprising a sequence with at least 80%, 85%, 90%, 91%, 92%, 93%, 94%, 95%, 96%, 97%, 98%, 99%, or 100% identity to a sequence comprising SEQ ID NO: 1238; a sequence with at least 80%, 85%, 90%, 91%, 92%, 93%, 94%, 95%, 96%, 97%, 98%, 99%, or 100% identity to a sequence comprising SEQ ID NO: 1239; a sequence with at least 80%, 85%, 90%, 91%, 92%, 93%, 94%, 95%, 96%, 97%, 98%, 99%, or 100% identity to a sequence comprising SEQ ID NO: 1240; a sequence with at least 80%, 85%, 90%, 91%, 92%, 93%, 94%, 95%, 96%, 97%, 98%, 99%, or 100% identity to a sequence comprising SEQ ID NO: 1241; or a sequence with at least 80%, 85%, 90%, 91%, 92%, 93%, 94%, 95%, 96%, 97%, 98%, 99%, or 100% identity to a sequence comprising SEQ ID NO: 1242.

In some aspects, the system is encoded by a nucleic acid comprising a sequence with at least 80%, 85%, 90%, 91%, 92%, 93%, 94%, 95%, 96%, 97%, 98%, 99%, or 100% identity to a sequence selected from the group consisting of the sequences set forth in SEQ ID NOs: 1120, 1121, 1122, 1123, 1124, 1238, 1239, 1240, 1241, or 1242.

Exemplary nucleic acids or expression plasmids encoding logic gate systems are provided in SEQ ID NOS: 1120, 1121, 1122, 1123, 1124, 1238, 1239, 1240, 1241, or 1242. Such exemplary nucleic acids or expression plasmids encoding logic gate systems include 5′ and 3′ nucleotides encoding a homology wing and sgRNA target sequence for cleavage of the expression plasmid and homology mediated insertion of the logic gate-encoding DNA into a cell genome (e.g., the 480 5′ nucleotides and the 473 3′ nucleotides of SEQ ID NOs: 1120, 1121, 1122, 1123, or 1124). An exemplary 5′ homology wing is provided in SEQ ID NO: 1235, and an exemplary 3′ homology wing is provided in SEQ ID NO: 1236.

Exemplary systems are provided in Table 18. In some aspects, the system is encoded by a nucleic acid comprising a sequence with at least 80%, 85%, 90%, 91%, 92%, 93%, 94%, 95%, 96%, 97%, 98%, 99%, or 100% identity to a sequence selected from the group provided in Table 18.

TABLE 18
Exemplary Logic Gate systems

| Name | Sequence |
|---|---|
| LG 219 | GAGCCATGCTTGGCTTACGAGGGCGACCAACCCATCAAACTCCCCGCCCCCAGCACTTTTATTTCTCCTC |
| (with | TTTAGGAAGTACACTTCAGTATCTTTGGCACAGTGCATGAGCACGACTAAAGTAAAACATCGCAGAAAAC |
| sgRNA | ATAGCTTTAGTCTACCCTTCGTGTCCTAAAAGGAAAACCAGTAGCTTCCCAGGCCACCGGAAGGGCAACA |
| and | CATGTCCTCTGCAGTTTCTGCACACGGGAAGGTAAAGACAGAGAGAGGACCTACTCCTCAACACAGAAAC |
| homology | ATTTCAAAATCTTTCCTCGCCTGCAACCCAAGCTGAAGTCATTCTCCCCAGAAATAACAAAAGTTGGAAG |
| wings) | AGAAGCCGGAGACAGGATAGGTGCAGGAAGCCCACACTTTGAGGGCAGCACTCAGACACCCTCTCCTGTG |
| SEQ ID | TGCAGGACGTGCCGAATGTTCAGGTGCAATGAGAATGAGCCATGCttggcttataaggtacgactgtgcc |
| NO: 1120 | ttctagttgccagccatctgttgtttgcccctcccccgtgccttccttgaccctggaaggtgccactccc |
| | actgtcctttcctaataaaatgaggaaattgcatcgcattgtctgagtaggtgtcattctattctggggg |
| | gtggggtggggcaggacagcaagggggaggattgggaagacaatagcaggcatgctggggatgcggtggg |
| | ctctatgggataagcttgatatcgaattcatcgatgttaataattaacatatatgttaatcattaacata |
| | tagttaattattaaccgctatgttaatgattaacaacggttaataattaacatatatgttaatcattaac |
| | atataactagtctagagggtatataatgggggccactagtctactaccagagTtcatcgctagcgctacc |
| | ggatccgccaccATGGCCCTGCCAGTAACGGCTCTGCTGCTGCCACTTGCTCTGCTCCTCCATGCAGCCA |
| | GGCCTCAAGTGCAGCTGGTCCAGTCTGGGGCGGAAGTGAAAAAGCCCGGAGCTAGTGTAAAGGTGTCCTG |
| | TAAAGCCAGCGGCTACACCTTCACCGGTTATTACCTGCATTGGGTCCGGCAGGCTCCTGGCCAGGGCCTG |
| | GAGTGGATGGGCTGGATTTCCGCATATAACGGAAACACAAATTACGCCCAGAACCTGCAAGGCCGCGTGA |
| | CCATGACCAGGGACACAAGCACTAGCACTGTCTACATGGAGTTGTCTAGCTTGAGAAGCGAAGATACCGC |
| | TGTGTACTATTGCGCCCGACACTCTTACTCGGGCTCATACTCAACGCTACCCTATTATGGGATGGATGTT |
| | TGGGGTCAAGGGACAACGGTCACAGTATCCTCTGGAGGCGGTGGCAGCGGAGGAGGCGGGTCTGGAGGTG |
| | GTGGATCAGACATTCAGATGACCCAGTCACCAAGTTCCTTATCCGCAAGCGTTGGGGATCGTGTTACAAT |
| | TACTTGCAGGGCCTCGCAAGGGATCTCTAATTATCTCGCTTGGTACCAGCAGAAACCTGGGAAAGCACCC |
| | AAGCTGCTGATCTACACTGCAAGCACACTTTTTCCAGGAGTGCCGTCAAGATTCTCTGGGTCCGGGAGTG |
| | GCACTGACTTCACCCTTACCATCTCCTCCCTCCAGCCTGAGGACTTTGCCACATATTATTGTCAACAGAG |
| | TTACTCCATACCACTCACGTTTGGCGGCGGAACAAAaGTtGAAATCAAGGCGGCAGCAaccacgacgcca |
| | gcgccgcgaccaccaacaccggcgcccaccatcgcgtcgcagccactgtcactgcgcccagaagcgtgcc |
| | ggccagcggcggggggcgcagtgcaTacgagggggctggacttcgcctgtgatatctacatctgggcgcc |
| | cttggccgggacttgtggggtccttctcctgtcactggttatcaccctttactgcaaacggggcagaaag |
| | aaactcctgtatatattcaaacaaccatttatgagaccagtacaaactactcaagaagaggacggctgta |
| | gctgccgatttccagaagaagaagaaggaggatgtgaactgagagtgaagttcagcaggagcgcagacgc |
| | ccccgcgtaccagcagggccagaaccagctctataacgagctcaatctaggacgaagagaggagtacgat |
| | gttttggacaagaggcgtggccgggaccctgagatggggggaaagccgagaaggaagaaccctcaggaag |
| | gcctgtacaatgaactgcagaaagataagatggcggaggcctacagtgagattgggatgaaaggcgagcg |
| | ccggaggggcaaggggcacgatggcctttaccagggtctcagtacagccaccaaggacacctacgacgcc |
| | cttcacatgcaggccctgccccctaggtaaaatcaacctctggattacaaaatttgtgaaagattgactg |
| | gtattcttaactatgttgctccttttacgctatgtggatacgctgctttaatgcctttgtatcatgctat |
| | tgcttcccgtatggctttcattttctcctccttgtataaatcctggttgctgtctctttatgaggagttg |
| | tggcccgttgtcaggcaacgtggcgtggtCtgcactgtgtttgctgacgcaacccccactggttggggca |
| | ttgccaccacctgtcagctcctttccgggactttcgctttccccctccctattgccacggcggaactcat |
| | cgccgcctgccttgcccgctgctggacaggggctcggctgttgggcactgacaattccgtggtgttgtcg |
| | gggaaatcatcgtcctttccttggctgctcgcctgtgttgccacctggattctgcgcgggacgtccttct |
| | gctacgtcccttcggccctcaatccagcggaccttccttcccgcggcctgctgccggctctgcggcctct |
| | tccgcgtcttcgccttcgccctcagacgagtcggatctccctttgggccgcctccccgcctggatccttg |
| | acttgcggccaacttgtttattgcagcttataatggttacaaataaagcaatagcatcacaaatttcaca |
| | aataaagcatttttttcactgcattctagttgtggtttgtccaaactcatcaatgtatcttatcatgtct |
| | gggatccttgacttgcggccgcaactcccacctgcaacatgcgtgactgactgaggccgcgactctagag |
| | tcgaccggatctgcgatcgctccggtgcccgtcagtgggcagagcgcacatcgcccacagtccccgagaa |
| | gttggggggaggggtcggcaattgaacgggtgcctagagaaggtggcgcggggtaaactgggaaagtgat |
| | gtcgtgtactggctccgcctttttcccgagggtgggggagaaccgtatataagtgcagtagtcgccgtga |
| | acgttctttttcgcaacgggtttgccgccagaacacagctgaagcttcgaggggctcgcatctctccttc |
| | acgcgcccgccgccctacctgaggccgccatccacgccggttgagtcgcgttctgccgcctcccgcctgt |
| | ggtgcctcctgaactgcgtccgccgtctaggtaaGTcgactcgttggatccCCACTACCCGGATCAACGC |
| | CCTAGGTTTATGTTTGGATGAACTGACATACGCGTATCCGTCTTAAGAATCTTTTCAAACACTAGTAGTG |
| | AAATATATATTAAACTAGTGTTTGAAAAGATTCTTATTACGGTAACGCGGAATTCGCAACTATTTTATCA |
| | ATTTTTTGCGTCGACACTTCAAGGGGCTTGCGGCCGCAACCATCTCCATGGCTGTTTGAATGAGGCTTCA |
| | GTACTTTACAGAATCGTTGCCTGCACATCTTGGAAACACTTGCTGGGATTACTTCGACTTCTTAACCCAA |
| | CAGAAGGCTCGAGAAGGTATATTGCTGTTGACAGTGAGCGCCAGTGTGAAGCTCTTGTCAGATAGTGAAG |
| | CCACAGATGTATCTGACAAGAGCTTCACACTGATGCCTACTGCCTCGGACTTCAAGGGGCTAGAATTCGA |
| | GCAATTATCTTGTTTACTAAAACTGAATACCTTGCTATCTCTTTGATACATTTTTACAAAGCTGAATTAA |
| | AATGGTATAAATTAAATCACTTTGACGGTGACTTAGGAGTATGCCGGATCAACGCCCTAGGTTTATGTTT |
| | GGATGAACTGACATACGCGTATCCGTCTAAAAATCAATCTCATTTCCTGGTAGTGAAATATATATTAAAC |
| | CAGGAAATGAGATTGATTTTTTTACGGTAACGCGGAATTCGCAACTATTTTATCAATTTTTTGCGTCGAC |
| | GACTGTGACAGCAGAGTATGCCGGATCAACGCCCTAGGTTTATGTTTGGATGAACTGACATACGCGTATC |
| | CGTCTTATGTAAAAGACAAACAATGCGTAGTGAAATATATATTAAACGCATTGTTTGTCTTTTACATATT |
| | ACGGTAACGCGGAATTCGCAACTATTTTATCAATTTTTTGCGTCGACCGGAACTATCTTGAAGAGTAGTA |
| | GTGGactagtgtgacgctgctgacccctttctttcccttctACAGATCCAAGCTGTGACCGGCGCCTACa |
| | cctgcagcccaagcttaccatggccttaccagtgaccgccttgctcctgccgctggccttgctgctccac |
| | gccgccaggcctGATATTCAGATGACACAGAGCCCAAGTTCACTGTCAGCGAGTGTCGGAGATCGGGTTA |
| | CTATAACCTGCCGAGCATCTCAGGATATCAATAATTACCTGGCCTGGTATCAGCAAAAACCCGGCAAAGT |
| | CCCGAAGCTCCTGATATACGCAGCTTCAACACTGCAAAGCGGTGTGCCCTCGCGCTTTAGCGGCTCTGGC |
| | AGCGGGACAGATTTCACCCTGACGATTTCCTCCCTTCAACCCGAGGACGTGGCCACTTACTACTCCCTCA |
| | ACTATTACTCTGTACCCTGGACCTTTGGCCAAGGGACAAAGGTGGAAATCAAGGGCTCTACTAGCGGTTC |
| | AGGGAAACCTGGGAGTGGAGAGGGCTCGACCAAGGGACAGGTCCAGCTCGTGGAGTCTGGCGGTGGGCTG |
| | GTTCAGCCAGGGAGGAGTTTGCGGCTTTCCTGTACGGGAAGCGGATTCACCTTTGGTGACTATGCCATGA |
| | ACTGGGTCAGGCAGGCTCCTGGAAAAGGCCTAGAGTGGGTGGGTTTCATCAGAACCAAGCCATATGGCGG |
| | CACTACAGAATATGCAGCCAGCGTAAAAGGGAGATTCACGTTCAGCCGCGACGACTCTAAGTCAATAGCT |
| | TACCTTCAGATGAACTCCCTCAAGACCGAAGACACCGCCGTGTACTATTGTACTATGATCCCTGTGCTGC |
| | GTTTTTTAGAGTGGTTGCCTTGGGGACAAGGGACATTAGTTACTGTGTCCTCCGCAACCAcgacgccagc |
| | gccgcgaccaccaacaccggcgcccaccatcgcgtcgcagcccctgtccctgcgcccTgaggcgtgcttc |
| | atgtacgtggcggcggccgcctttgtgcttctgttcttcgtgggctgcggggtgctgctgtcccgtaaac |
| | gCagacgtcaacacggtcaactgtggtttccagaaggttttaaggtctccgaagcaagtaagaagaaaag |
| | acgtgaaccactgggagaagatagcgtcggtctgaaaccactcaagaatgccatggtttctaaactgagc |
| | cagctgcagacggagctcctggcggccctgctggagtcagggctgagcaaagaggcactgctccaggcac |
| | tgggCgagccggggccctacctcctggctggagaaggccccctggacaagggggagtcctgcggcggcgg |
| | tcgaggggagctggctgagctgcccaatgggctgggggagactcggggctccgaggacgagacCgacgac |
| | gatggggaagacttcacgccacccatcctcaaagagctggagaacctcagccctgaggaggcggcccacc |
| | agaaagccgtggtggagacccttctgcaggaggacccgtggcgtgtggcgaagatggtcaagtcctacct |
| | gcagcagcacaacatcccacagcgggaggtggtcgataccactggcctcaaccagtcccacctgtcccaa |
| | cacctcaacaagggcactcccatgaagacgcagaagcgggccgccctgtacacctggtaTgtccgcaagc |
| | agcgagaggtggcgcagcagttcacccatgcagggcagggagggctgattgaAgaGcccacaggAgatga |
| | gctaccaaccaagaaggggcggaggaaccgtttcaagtggggcccagcatcccagcagatcctgttccag |
| | gcctatgagaggcagaagaaccctagcaaggaggagcgagaAacgctagtggaggagtgcaatagggcgg |
| | aatgcatccagagaggTgtgtcAccatcacaAgcacaAggTctgggctccaacctcgtcacggaggtgcg |
| | tgtctacaactggtttgccaaccggcgcaaagaagaagccttccggcacaagctggccatgacctgcagg |
| | gatgagtttcccaccatggtgtttccttctgggcagatcagccaggcctcggccttggccccggcccctc |
| | cccaagtcctgccccaggctccagcccctgcccctgctccagccatggtatcagctctggcccaggcccc |
| | agcccctgtcccagtcctagccccaggccctcctcaAgctgtggccccacctgcccccaagcccacccaA |
| | gctggggaaggaacgctgtcagaggccctgctgcagctgcagtttgatgatgaagacctgggggccttgc |
| | ttggcaacagcacagacccagctgtgttcacagacctggcatccgtcgacaactccgagtttcagcagct |
| | gctgaaccagggcatacctgtggccccccacacaactgagcccatgctgatggagtaccctgaggctata |
| | actcgcctagtgacaggggcccagaggccccccgacccagctcctgctccactgggggccccggggctcc |
| | ccaatggcctcctttcaggagatgaagacttctcctccattgcggacatggacttctcagccctgctgag |
| | tcagatcagctccTAAAGGAaataaaagatctttaatgaaaatAGATCTGTGTGTTGGTTTTTTGTGTGa |
| | ataaaagatccagagctctagAGATCTGTGTGTTGGTTTTTTGTGTGCGAGGGCAATCTGGCCCATCAAG |
| | TGGCCTTCGCCTCTGGGAGTAACAAAAATGCACTTCAAAATAGCTTCTGTAATCAAGCTGCATGGGTGGA |
| | GTACTCCCCAGCTGACTCCAGGAAGTTCTCTATCCAAAGCTATTCATTAGGCCAGAGCTGTGCAAATAAT |
| | TAGTCACCCACTTGCTCCATAACCCTCCATGACAGCCCAGGCATTGAGTCCAGGTGGGACCATCAAGCCA |
| | TGCTCTGGTGGCTCATGCATTATCATAGAAATGGGAGGCTTTATTTATTTTACTAAAAAGAACAAAAACA |
| | ACAGACTGCTGTCCTTTAGACAATAGGATCACGTCATCTGAGCCCTCTGTGCCCCAGGTGACAAGCCCAG |
| | CCCCAAGTTCTCTTTCCTCAGCCTCCCCACACATGTTCTGGAGGAGATGGGCCCAGCAGGCTGCTCTGAG |
| | GCCTGGCCCCTCGTAAGCCAAGCATGGCTC |
| LG 239 | GAGCCATGCTTGGCTTACGAGGGCGACCAACCCATCAAACTCCCCGCCCCCAGCACTTTTATTTCTCCTC |
| (with | TTTAGGAAGTACACTTCAGTATCTTTGGCACAGTGCATGAGCACGACTAAAGTAAAACATCGCAGAAAAC |
| sgRNA | ATAGCTTTAGTCTACCCTTCGTGTCCTAAAAGGAAAACCAGTAGCTTCCCAGGCCACCGGAAGGGCAACA |
| and | CATGTCCTCTGCAGTTTCTGCACACGGGAAGGTAAAGACAGAGAGAGGACCTACTCCTCAACACAGAAAC |
| homology | ATTTCAAAATCTTTCCTCGCCTGCAACCCAAGCTGAAGTCATTCTCCCCAGAAATAACAAAAGTTGGAAG |
| wings) | AGAAGCCGGAGACAGGATAGGTGCAGGAAGCCCACACTTTGAGGGCAGCACTCAGACACCCTCTCCTGTG |
| SEQ ID | TGCAGGACGTGCCGAATGTTCAGGTGCAATGAGAATGAGCCATGCTTGGCTTATAaGGTAcgactgtgcc |
| NO: 1121 | ttctagttgccagccatctgttgtttgcccctcccccgtgccttccttgaccctggaaggtgccactccc |
| | actgtcctttcctaataaaatgaggaaattgcatcgcattgtctgagtaggtgtcattctattctggggg |
| | gtggggtggggcaggacagcaagggggaggattgggaagacaatagcaggcatgctggggatgcggtggg |
| | ctctatgggataagcttgatatcgaattcatcgatgttaataattaacatatatgttaatcattaacata |
| | tagttaattattaaccgctatgttaatgattaacaacggttaataattaacatatatgttaatcattaac |
| | atataactagtctagagggtatataatgggggccactagtctactaccagagTtcatcgctagcgctacc |
| | ggatccgccaccATGGCCCTGCCAGTAACGGCTCTGCTGCTGCCACTTGCTCTGCTCCTCCATGCAGCCA |
| | GGCCTCAGGTCCAGTTGGTACAGAGCGGCGCCGAAGTGAAAAAGCCTGGGGCGTCCGTCAAAGTGTCTTG |
| | CAAGGCCTCCGGCTATACATTCACCGGGTACTACATGCATTGGGTGCGGCAGGCACCTGGCCAGGGTCTA |
| | GAATGGATGGGCCGGATCAATCCCAACTCCGGCGGCACAAACTATGCTCAGAAATTTCAAGGTCGCGTCA |
| | CCATGACCCGTGACACAAGTACGAGCACCGTCTACATGGAGCTGTCCTCCCTCAGGAGCGAGGATACAGC |
| | CGTGTACTATTGTGCAAGGGAGCGCGCCGGCTATAGCAGCGGGCAGTTCGATTATTGGGGACAAGGGACT |
| | CTGGTAACTGTGTCCTCCGGAGGCGGAGGATCAGGCGGAGGAGGCTCAGGAGGTGGAGGTTCTGACATTC |
| | AGATGACTCAATCTCCCTCGTCACTGTCAGCTAGTGTTGGGGATAGAGTGACTATTACCTGCCGAGCCAG |
| | TCAGTCAATATCTAACTGGCTCGCATGGTACCAGCAGAAGCCAGGGAAGGCTCCCAAACTGCTGATCTAC |
| | GCCGCGAGCACCCTTCAGAATGGCGTGCCGTCTAGATTTAGCGGTTCTGGGTCTGGGACCGACTTTACAC |
| | TTACTATCAGTAGTTTACAACCAGAGGACTTTGCTACTTATTACTGTCAACAGAGCTACACCTTCCCTAT |
| | TACGTTCGGCCAGGGAACAAAAGTTGAAATCAAGGCGGCAGCAaccacgacgccagcgccgcgaccacca |
| | acaccggcgcccaccatcgcgtcgcagccactgtcactgcgcccagaagcgtgccggccagcggcggggg |
| | gcgcagtgcaTacgagggggctggacttcgcctgtgatatctacatctgggcgcccttggccgggacttg |
| | tggggtccttctcctgtcactggttatcaccctttactgcaaacggggcagaaagaaactcctgtatata |
| | ttcaaacaaccatttatgagaccagtacaaactactcaagaagaggacggctgtagctgccgatttccag |
| | aagaagaagaaggaggatgtgaactgagagtgaagttcagcaggagcgcagacgcccccgcgtaccagca |
| | gggccagaaccagctctataacgagctcaatctaggacgaagagaggagtacgatgttttggacaagagg |
| | cgtggccgggaccctgagatggggggaaagccgagaaggaagaaccctcaggaaggcctgtacaatgaac |
| | tgcagaaagataagatggcggaggcctacagtgagattgggatgaaaggcgagcgccggaggggcaaggg |
| | gcacgatggcctttaccagggtctcagtacagccaccaaggacacctacgacgcccttcacatgcaggcc |
| | ctgccccctaggtaaaatcaacctctggattacaaaatttgtgaaagattgactggtattcttaactatg |
| | ttgctccttttacgctatgtggatacgctgctttaatgcctttgtatcatgctattgcttcccgtatggc |
| | tttcattttctcctccttgtataaatcctggttgctgtctctttatgaggagttgtggcccgttgtcagg |
| | caacgtggcgtggtCtgcactgtgtttgctgacgcaacccccactggttggggcattgccaccacctgtc |
| | agctcctttccgggactttcgctttccccctccctattgccacggcggaactcatcgccgcctgccttgc |
| | ccgctgctggacaggggctcggctgttgggcactgacaattccgtggtgttgtcggggaaatcatcgtcc |
| | tttccttggctgctcgcctgtgttgccacctggattctgcgcgggacgtccttctgctacgtcccttcgg |
| | ccctcaatccagcggaccttccttcccgcggcctgctgccggctctgcggcctcttccgcgtcttcgcct |
| | tcgccctcagacgagtcggatctccctttgggccgcctccccgcctggatccttgacttgcggccaactt |
| | gtttattgcagcttataatggttacaaataaagcaatagcatcacaaatttcacaaataaagcatttttt |
| | tcactgcattctagttgtggtttgtccaaactcatcaatgtatcttatcatgtctgggatccttgacttg |
| | cggccgcaactcccacctgcaacatgcgtgactgactgaggccgcgactctagagtcgaccggatctgcg |
| | atcgctccggtgcccgtcagtgggcagagcgcacatcgcccacagtccccgagaagttggggggaggggt |
| | cggcaattgaacgggtgcctagagaaggtggcgcggggtaaactgggaaagtgatgtcgtgtactggctc |
| | cgcctttttcccgagggtgggggagaaccgtatataagtgcagtagtcgccgtgaacgttctttttcgca |
| | acgggtttgccgccagaacacagctgaagcttcgaggggctcgcatctctccttcacgcgcccgccgccc |
| | tacctgaggccgccatccacgccggttgagtcgcgttctgccgcctcccgcctgtggtgcctcctgaact |
| | gcgtccgccgtctaggtaaGTcgactcgttggatccCCACTACCCGGATCAACGCCCTAGGTTTATGTTT |
| | GGATGAACTGACATACGCGTATCCGTCTTAAGAATCTTTTCAAACACTAGTAGTGAAATATATATTAAAC |
| | TAGTGTTTGAAAAGATTCTTATTACGGTAACGCGGAATTCGCAACTATTTTATCAATTTTTTGCGTCGAC |
| | ACTTCAAGGGGCTTGCGGCCGCAACCATCTCCATGGCTGTTTGAATGAGGCTTCAGTACTTTACAGAATC |
| | GTTGCCTGCACATCTTGGAAACACTTGCTGGGATTACTTCGACTTCTTAACCCAACAGAAGGCTCGAGAA |
| | GGTATATTGCTGTTGACAGTGAGCGCCAGTGTGAAGCTCTTGTCAGATAGTGAAGCCACAGATGTATCTG |
| | ACAAGAGCTTCACACTGATGCCTACTGCCTCGGACTTCAAGGGGCTAGAATTCGAGCAATTATCTTGTTT |
| | ACTAAAACTGAATACCTTGCTATCTCTTTGATACATTTTTACAAAGCTGAATTAAAATGGTATAAATTAA |
| | ATCACTTTGACGGTGACTTAGGAGTATGCCGGATCAACGCCCTAGGTTTATGTTTGGATGAACTGACATA |
| | CGCGTATCCGTCTAAAAATCAATCTCATTTCCTGGTAGTGAAATATATATTAAACCAGGAAATGAGATTG |
| | ATTTTTTTACGGTAACGCGGAATTCGCAACTATTTTATCAATTTTTTGCGTCGACGACTGTGACAGCAGA |
| | GTATGCCGGATCAACGCCCTAGGTTTATGTTTGGATGAACTGACATACGCGTATCCGTCTTATGTAAAAG |
| | ACAAACAATGCGTAGTGAAATATATATTAAACGCATTGTTTGTCTTTTACATATTACGGTAACGCGGAAT |
| | TCGCAACTATTTTATCAATTTTTTGCGTCGACCGGAACTATCTTGAAGAGTAGTAGTGGactagtgtgac |
| | gctgctgacccctttctttcccttctACAGATCCAAGCTGTGACCGGCGCCTACacctgcagcccaagct |
| | taccatggccttaccagtgaccgccttgctcctgccgctggccttgctgctccacgccgccaggcctGAG |
| | ATAGTCCTGACCCAGTCACCCGATTTCCAGAGTGTTACTCCTAAGGAGAAAGTGACTATAACATGCCGGG |
| | CATCTCAGTCTGTCGGAAGCGGGCTACATTGGTACCAGCAGAAGCCTGACCAGAGCCCGAAACTGCTCAT |
| | CAAATATGCCTCCCAGTCGTTTTCTGGCGTGCCCTCTCGCTTTTCCGGAAGCGGATCTGGCACAGACTTC |
| | ACCTTGACCATCAATAGCCTGGAAACTGAGGACGCCGCTACGTATTTCTGCCAGCAGTCCTCCAGTCTGC |
| | CTTGGACATTTGGTCAGGGAACGAAGGTGGAGATCAAGGGTTCAACATCAGGGAGCGGGAAACCGGGCTC |
| | TGGCGAGGGCTCAACAAAGGGACAAGTGCAACTGCAAGAATCGGGACCCGGGCTGGTTAAACCAAGTGAA |
| | ACCCTTTCCCTCACTTGTACCGTAAGCGGCGGTAGCGTGTCCTCTGGTAGCTACTATTGGAGTTGGATTA |
| | GGCAGGCGCCAGGGAAAGGCCTCGAATGGATTGGGTATATCTACTACAGCGGCAGTAACTACTACAACCC |
| | ATCATTGAAGTCTAGAGTGACAATTAGTGTCGATACCTCTAAAAATCAATTCTCACTTAAGCTGCGAGCT |
| | GTAACCGCCGCAGACACTGCGGTGTACTATTGTGCCCGTTGGATGACCACTATCAAGGGCTACTTCGATT |
| | ATTGGGGACAAGGGACATTAGTTACGGTGTCCTCCGCAACCAcgacgccagcgccgcgaccaccaacacc |
| | ggcgcccaccatcgcgtcgcagcccctgtccctgcgcccTgaggcgtgcttcatgtacgtggcggcggcc |
| | gcctttgtgcttctgttcttcgtgggctgcggggtgctgctgtcccgtaaacgCagacgtcaacacggtc |
| | aactgtggtttccagaaggttttaaggtctccgaagcaagtaagaagaaaagacgtgaaccactgggaga |
| | agatagcgtcggtctgaaaccactcaagaatgccatggtttctaaactgagccagctgcagacggagctc |
| | ctggcggccctgctggagtcagggctgagcaaagaggcactgctccaggcactgggCgagccggggccct |
| | acctcctggctggagaaggccccctggacaagggggagtcctgcggcggcggtcgaggggagctggctga |
| | gctgcccaatgggctgggggagactcggggctccgaggacgagacCgacgacgatggggaagacttcacg |
| | ccacccatcctcaaagagctggagaacctcagccctgaggaggcggcccaccagaaagccgtggtggaga |
| | cccttctgcaggaggacccgtggcgtgtggcgaagatggtcaagtcctacctgcagcagcacaacatccc |
| | acagcgggaggtggtcgataccactggcctcaaccagtcccacctgtcccaacacctcaacaagggcact |
| | cccatgaagacgcagaagcgggccgccctgtacacctggtaTgtccgcaagcagcgagaggtggcgcagc |
| | agttcacccatgcagggcagggagggctgattgaAgaGcccacaggAgatgagctaccaaccaagaaggg |
| | gcggaggaaccgtttcaagtggggcccagcatcccagcagatcctgttccaggcctatgagaggcagaag |
| | aaccctagcaaggaggagcgagaAacgctagtggaggagtgcaatagggcggaatgcatccagagaggTg |
| | tgtcAccatcacaAgcacaAggTctgggctccaacctcgtcacggaggtgcgtgtctacaactggtttgc |
| | caaccggcgcaaagaagaagccttccggcacaagctggccatgacctgcagggatgagtttcccaccatg |
| | gtgtttccttctgggcagatcagccaggcctcggccttggccccggcccctccccaagtcctgccccagg |
| | ctccagcccctgcccctgctccagccatggtatcagctctggcccaggccccagcccctgtcccagtcct |
| | agccccaggccctcctcaAgctgtggccccacctgcccccaagcccacccaAgctggggaaggaacgctg |
| | tcagaggccctgctgcagctgcagtttgatgatgaagacctgggggccttgcttggcaacagcacagacc |
| | cagctgtgttcacagacctggcatccgtcgacaactccgagtttcagcagctgctgaaccagggcatacc |
| | tgtggccccccacacaactgagcccatgctgatggagtaccctgaggctataactcgcctagtgacaggg |
| | gcccagaggccccccgacccagctcctgctccactgggggccccggggctccccaatggcctcctttcag |
| | gagatgaagacttctcctccattgcggacatggacttctcagccctgctgagtcagatcagctccTAAAG |
| | GAaataaaagatctttaatgaaaatAGATCTGTGTGTTGGTTTTTTGTGTGaataaaagatccagagctc |
| | tagAGATCTGTGTGTTGGTTTTTTGTGTGCGAGGGCAATCTGGCCCATCAAGTGGCCTTCGCCTCTGGGA |
| | GTAACAAAAATGCACTTCAAAATAGCTTCTGTAATCAAGCTGCATGGGTGGAGTACTCCCCAGCTGACTC |
| | CAGGAAGTTCTCTATCCAAAGCTATTCATTAGGCCAGAGCTGTGCAAATAATTAGTCACCCACTTGCTCC |
| | ATAACCCTCCATGACAGCCCAGGCATTGAGTCCAGGTGGGACCATCAAGCCATGCTCTGGTGGCTCATGC |
| | ATTATCATAGAAATGGGAGGCTTTATTTATTTTACTAAAAAGAACAAAAACAACAGACTGCTGTCCTTTA |
| | GACAATAGGATCACGTCATCTGAGCCCTCTGTGCCCCAGGTGACAAGCCCAGCCCCAAGTTCTCTTTCCT |
| | CAGCCTCCCCACACATGTTCTGGAGGAGATGGGCCCAGCAGGCTGCTCTGAGGCCTGGCCCCTCGTAAGC |
| | CAAGCATGGCTC |
| LG 39 | GAGCCATGCTTGGCTTACGAGGGCGACCAACCCATCAAACTCCCCGCCCCCAGCACTTTTATTTCTCCTC |
| (with | TTTAGGAAGTACACTTCAGTATCTTTGGCACAGTGCATGAGCACGACTAAAGTAAAACATCGCAGAAAAC |
| sgRNA | ATAGCTTTAGTCTACCCTTCGTGTCCTAAAAGGAAAACCAGTAGCTTCCCAGGCCACCGGAAGGGCAACA |
| and | CATGTCCTCTGCAGTTTCTGCACACGGGAAGGTAAAGACAGAGAGAGGACCTACTCCTCAACACAGAAAC |
| homology | ATTTCAAAATCTTTCCTCGCCTGCAACCCAAGCTGAAGTCATTCTCCCCAGAAATAACAAAAGTTGGAAG |
| wings) | AGAAGCCGGAGACAGGATAGGTGCAGGAAGCCCACACTTTGAGGGCAGCACTCAGACACCCTCTCCTGTG |
| SEQ ID | TGCAGGACGTGCCGAATGTTCAGGTGCAATGAGAATGAGCCATGCTTGGCTTATAaGGTAcgactgtgcc |
| NO: 1122 | ttctagttgccagccatctgttgtttgcccctcccccgtgccttccttgaccctggaaggtgccactccc |
| | actgtcctttcctaataaaatgaggaaattgcatcgcattgtctgagtaggtgtcattctattctggggg |
| | gtggggtggggcaggacagcaagggggaggattgggaagacaatagcaggcatgctggggatgcggtggg |
| | ctctatgggataagcttgatatcgaattcatcgatgttaataattaacatatatgttaatcattaacata |
| | tagttaattattaaccgctatgttaatgattaacaacggttaataattaacatatatgttaatcattaac |
| | atataactagtctagagggtatataatgggggccactagtctactaccagagTtcatcgctagcgctacc |
| | ggatccgccaccATGGCCCTGCCAGTAACGGCTCTGCTGCTGCCACTTGCTCTGCTCCTCCATGCAGCCA |
| | GGCCTCAGGTCCAGTTGGTACAGAGCGGCGCCGAAGTGAAAAAGCCTGGGGCGTCCGTCAAAGTGTCTTG |
| | CAAGGCCTCCGGCTATACATTCACCGGGTACTACATGCATTGGGTGCGGCAGGCACCTGGCCAGGGTCTA |
| | GAATGGATGGGCCGGATCAATCCCAACTCCGGCGGCACAAACTATGCTCAGAAATTTCAAGGTCGCGTCA |
| | CCATGACCCGTGACACAAGTACGAGCACCGTCTACATGGAGCTGTCCTCCCTCAGGAGCGAGGATACAGC |
| | CGTGTACTATTGTGCAAGGGAGCGCGCCGGCTATAGCAGCGGGCAGTTCGATTATTGGGGACAAGGGACT |
| | CTGGTAACTGTGTCCTCCGGAGGCGGAGGATCAGGCGGAGGAGGCTCAGGAGGTGGAGGTTCTGACATTC |
| | AGATGACTCAATCTCCCTCGTCACTGTCAGCTAGTGTTGGGGATAGAGTGACTATTACCTGCCGAGCCAG |
| | TCAGTCAATATCTAACTGGCTCGCATGGTACCAGCAGAAGCCAGGGAAGGCTCCCAAACTGCTGATCTAC |
| | GCCGCGAGCACCCTTCAGAATGGCGTGCCGTCTAGATTTAGCGGTTCTGGGTCTGGGACCGACTTTACAC |
| | TTACTATCAGTAGTTTACAACCAGAGGACTTTGCTACTTATTACTGTCAACAGAGCTACACCTTCCCTAT |
| | TACGTTCGGCCAGGGAACAAAAGTTGAAATCAAGGCGGCAGCAaccacgacgccagcgccgcgaccacca |
| | acaccggcgcccaccatcgcgtcgcagccactgtcactgcgcccagaagcgtgccggccagcggcggggg |
| | gcgcagtgcaTacgagggggctggacttcgcctgtgatatctacatctgggcgcccttggccgggacttg |
| | tggggtccttctcctgtcactggttatcaccctttactgcaaacggggcagaaagaaactcctgtatata |
| | ttcaaacaaccatttatgagaccagtacaaactactcaagaagaggacggctgtagctgccgatttccag |
| | aagaagaagaaggaggatgtgaactgagagtgaagttcagcaggagcgcagacgcccccgcgtaccagca |
| | gggccagaaccagctctataacgagctcaatctaggacgaagagaggagtacgatgttttggacaagagg |
| | cgtggccgggaccctgagatggggggaaagccgagaaggaagaaccctcaggaaggcctgtacaatgaac |
| | tgcagaaagataagatggcggaggcctacagtgagattgggatgaaaggcgagcgccggaggggcaaggg |
| | gcacgatggcctttaccagggtctcagtacagccaccaaggacacctacgacgcccttcacatgcaggcc |
| | ctgccccctaggtaaaatcaacctctggattacaaaatttgtgaaagattgactggtattcttaactatg |
| | ttgctccttttacgctatgtggatacgctgctttaatgcctttgtatcatgctattgcttcccgtatggc |
| | tttcattttctcctccttgtataaatcctggttgctgtctctttatgaggagttgtggcccgttgtcagg |
| | caacgtggcgtggtCtgcactgtgtttgctgacgcaacccccactggttggggcattgccaccacctgtc |
| | agctcctttccgggactttcgctttccccctccctattgccacggcggaactcatcgccgcctgccttgc |
| | ccgctgctggacaggggctcggctgttgggcactgacaattccgtggtgttgtcggggaaatcatcgtcc |
| | tttccttggctgctcgcctgtgttgccacctggattctgcgcgggacgtccttctgctacgtcccttcgg |
| | ccctcaatccagcggaccttccttcccgcggcctgctgccggctctgcggcctcttccgcgtcttcgcct |
| | tcgccctcagacgagtcggatctccctttgggccgcctccccgcctggatccttgacttgcggccaactt |
| | gtttattgcagcttataatggttacaaataaagcaatagcatcacaaatttcacaaataaagcatttttt |
| | tcactgcattctagttgtggtttgtccaaactcatcaatgtatcttatcatgtctgggatccttgacttg |
| | cggccgcaactcccacctgcaacatgcgtgactgactgaggccgcgactctagagtcgaccggatctgcg |
| | atcgctccggtgcccgtcagtgggcagagcgcacatcgcccacagtccccgagaagttggggggaggggt |
| | cggcaattgaacgggtgcctagagaaggtggcgcggggtaaactgggaaagtgatgtcgtgtactggctc |
| | cgcctttttcccgagggtgggggagaaccgtatataagtgcagtagtcgccgtgaacgttctttttcgca |
| | acgggtttgccgccagaacacagctgaagcttcgaggggctcgcatctctccttcacgcgcccgccgccc |
| | tacctgaggccgccatccacgccggttgagtcgcgttctgccgcctcccgcctgtggtgcctcctgaact |
| | gcgtccgccgtctaggtaaGTcgactcgttggatccCCACTACCCGGATCAACGCCCTAGGTTTATGTTT |
| | GGATGAACTGACATACGCGTATCCGTCTTAAGAATCTTTTCAAACACTAGTAGTGAAATATATATTAAAC |
| | TAGTGTTTGAAAAGATTCTTATTACGGTAACGCGGAATTCGCAACTATTTTATCAATTTTTTGCGTCGAC |
| | ACTTCAAGGGGCTTGCGGCCGCAACCATCTCCATGGCTGTTTGAATGAGGCTTCAGTACTTTACAGAATC |
| | GTTGCCTGCACATCTTGGAAACACTTGCTGGGATTACTTCGACTTCTTAACCCAACAGAAGGCTCGAGAA |
| | GGTATATTGCTGTTGACAGTGAGCGCCAGTGTGAAGCTCTTGTCAGATAGTGAAGCCACAGATGTATCTG |
| | ACAAGAGCTTCACACTGATGCCTACTGCCTCGGACTTCAAGGGGCTAGAATTCGAGCAATTATCTTGTTT |
| | ACTAAAACTGAATACCTTGCTATCTCTTTGATACATTTTTACAAAGCTGAATTAAAATGGTATAAATTAA |
| | ATCACTTTGACGGTGACTTAGGAGTATGCCGGATCAACGCCCTAGGTTTATGTTTGGATGAACTGACATA |
| | CGCGTATCCGTCTAAAAATCAATCTCATTTCCTGGTAGTGAAATATATATTAAACCAGGAAATGAGATTG |
| | ATTTTTTTACGGTAACGCGGAATTCGCAACTATTTTATCAATTTTTTGCGTCGACGACTGTGACAGCAGA |
| | GTATGCCGGATCAACGCCCTAGGTTTATGTTTGGATGAACTGACATACGCGTATCCGTCTTATGTAAAAG |
| | ACAAACAATGCGTAGTGAAATATATATTAAACGCATTGTTTGTCTTTTACATATTACGGTAACGCGGAAT |
| | TCGCAACTATTTTATCAATTTTTTGCGTCGACCGGAACTATCTTGAAGAGTAGTAGTGGactagtgtgac |
| | gctgctgacccctttctttcccttctACAGATCCAAGCTGTGACCGGCGCCTACacctgcagcccaagct |
| | taccatggccttaccagtgaccgccttgctcctgccgctggccttgctgctccacgccgccaggcctGAA |
| | ATCGTCCTGACACAGTCTCCAGATTTTCAGAGCGTGACGCCAAAGGAGAAAGTGACAATTACATGCCGGG |
| | CATCTCAGTCTGTTGGGTCTGGGTTGCATTGGTATCAGCAAAAGCCCGACCAGTCACCCAAACTGCTCAT |
| | CAAATATGCAAGCCAGAGTTTTTCAGGCGTACCTTCACGATTTAGCGGAAGTGGTTCTGGCACTGACTTC |
| | ACCTTGACGATTAATAGCCTGGAAGTAGAAGACGCTGCCACTTTCTACTGCCTGCAAAGTAGCTCCCTGC |
| | CCTGGACTTTTGGGCAGGGTACTAAGGTCGAGATCAAGGGCTCGACAAGCGGAAGTGGCAAACCGGGCAG |
| | CGGCGAGGGAAGCACCAAGGGACAAGTGCAACTGCAAGAGTCTGGACCCGGGCTGGTGAAACCAAGCGAG |
| | ACATTATCCCTCACTTGTACCGTGTCAGGCGGTAGTGTGTCCTCCGGGAATTTCTACTGGAGTTGGATAC |
| | GCCAGCCTCCTGGGAAGGGCCTTGAATGGATTGGCTACATCTACTATTCAGGCTCCACCTACTACAACCC |
| | GTCTTTGAAGTCAAGGGTTACGATAAGCGTCGATACCTCCAAAAACCAATTCTCCCTAAAGCTCAGATCG |
| | TTAACTGCCGCTGATACCGCGGTGTACTATTGTGCCCGTTGGATGACCAAAGTTAAGGGTTATTTCGACT |
| | ATTGGGGACAAGGGACACTTGTCACCGTGTCCTCCGCAACCAcgacgccagcgccgcgaccaccaacacc |
| | ggcgcccaccatcgcgtcgcagcccctgtccctgcgcccTgaggcgtgcttcatgtacgtggcggcggcc |
| | gcctttgtgcttctgttcttcgtgggctgcggggtgctgctgtcccgtaaacgCagacgtcaacacggtc |
| | aactgtggtttccagaaggttttaaggtctccgaagcaagtaagaagaaaagacgtgaaccactgggaga |
| | agatagcgtcggtctgaaaccactcaagaatgccatggtttctaaactgagccagctgcagacggagctc |
| | ctggcggccctgctggagtcagggctgagcaaagaggcactgctccaggcactgggCgagccggggccct |
| | acctcctggctggagaaggccccctggacaagggggagtcctgcggcggcggtcgaggggagctggctga |
| | gctgcccaatgggctgggggagactcggggctccgaggacgagacCgacgacgatggggaagacttcacg |
| | ccacccatcctcaaagagctggagaacctcagccctgaggaggcggcccaccagaaagccgtggtggaga |
| | cccttctgcaggaggacccgtggcgtgtggcgaagatggtcaagtcctacctgcagcagcacaacatccc |
| | acagcgggaggtggtcgataccactggcctcaaccagtcccacctgtcccaacacctcaacaagggcact |
| | cccatgaagacgcagaagcgggccgccctgtacacctggtaTgtccgcaagcagcgagaggtggcgcagc |
| | agttcacccatgcagggcagggagggctgattgaAgaGcccacaggAgatgagctaccaaccaagaaggg |
| | gcggaggaaccgtttcaagtggggcccagcatcccagcagatcctgttccaggcctatgagaggcagaag |
| | aaccctagcaaggaggagcgagaAacgctagtggaggagtgcaatagggcggaatgcatccagagaggTg |
| | tgtcAccatcacaAgcacaAggTctgggctccaacctcgtcacggaggtgcgtgtctacaactggtttgc |
| | caaccggcgcaaagaagaagccttccggcacaagctggccatgacctgcagggatgagtttcccaccatg |
| | gtgtttccttctgggcagatcagccaggcctcggccttggccccggcccctccccaagtcctgccccagg |
| | ctccagcccctgcccctgctccagccatggtatcagctctggcccaggccccagcccctgtcccagtcct |
| | agccccaggccctcctcaAgctgtggccccacctgcccccaagcccacccaAgctggggaaggaacgctg |
| | tcagaggccctgctgcagctgcagtttgatgatgaagacctgggggccttgcttggcaacagcacagacc |
| | cagctgtgttcacagacctggcatccgtcgacaactccgagtttcagcagctgctgaaccagggcatacc |
| | tgtggccccccacacaactgagcccatgctgatggagtaccctgaggctataactcgcctagtgacaggg |
| | gcccagaggccccccgacccagctcctgctccactgggggccccggggctccccaatggcctcctttcag |
| | gagatgaagacttctcctccattgcggacatggacttctcagccctgctgagtcagatcagctccTAAAG |
| | GACGGGTGGCATCCCTGTGACCCCTCCCCAGTGCCTCTCCTGGCCCTGGAAGTTGCCACTCCAGTGCCCA |
| | CCAGCCTTGTCCTAATAAAATTAAGTTGCATCATTTTGTCTGACTAGGTGTCCTTCTATAATATTATGGG |
| | GTGGAGGGGGGTGGTATGGAGCAAGGGGCAAGTTGGGAAGACAACCTGTAGGGCCTGCGGGGTCTATTGG |
| | GAACCAAGCTGGAGTGCAGTGGCACAATCTTGGCTCACTGCAATCTCCGCCTCCTGGGTTCAAGCGATTC |
| | TCCTGCCTCAGCCTCCCGAGTTGTTGGGATTCCAGGCATGCATGACCAGGCTCAGCTAATTTTTGTTTTT |
| | TTGGTAGAaACGGGGTTTCACCATATTGGCCAGGCTGGTCTCCAACTCCTAATCTCAGGTGATCTACCCA |
| | CCTTGGCCTCCCAAATTGCTGGGATTACAGGCGTGAACCACTGCTCCCttccctgtccttcCGAGGGCAA |
| | TCTGGCCCATCAAGTGGCCTTCGCCTCTGGGAGTAACAAAAATGCACTTCAAAATAGCTTCTGTAATCAA |
| | GCTGCATGGGTGGAGTACTCCCCAGCTGACTCCAGGAAGTTCTCTATCCAAAGCTATTCATTAGGCCAGA |
| | GCTGTGCAAATAATTAGTCACCCACTTGCTCCATAACCCTCCATGACAGCCCAGGCATTGAGTCCAGGTG |
| | GGACCATCAAGCCATGCTCTGGTGGCTCATGCATTATCATAGAAATGGGAGGCTTTATTTATTTTACTAA |
| | AAAGAACAAAAACAACAGACTGCTGTCCTTTAGACAATAGGATCACGTCATCTGAGCCCTCTGTGCCCCA |
| | GGTGACAAGCCCAGCCCCAAGTTCTCTTTCCTCAGCCTCCCCACACATGTTCTGGAGGAGATGGGCCCAG |
| | CAGGCTGCTCTGAGGCCTGGCCCCTCGTAAGCCAAGCATGGCTC |
| LG 43 | GAGCCATGCTTGGCTTACGAGGGCGACCAACCCATCAAACTCCCCGCCCCCAGCACTTTTATTTCTCCTC |
| (with | TTTAGGAAGTACACTTCAGTATCTTTGGCACAGTGCATGAGCACGACTAAAGTAAAACATCGCAGAAAAC |
| sgRNA | ATAGCTTTAGTCTACCCTTCGTGTCCTAAAAGGAAAACCAGTAGCTTCCCAGGCCACCGGAAGGGCAACA |
| and | CATGTCCTCTGCAGTTTCTGCACACGGGAAGGTAAAGACAGAGAGAGGACCTACTCCTCAACACAGAAAC |
| homology | ATTTCAAAATCTTTCCTCGCCTGCAACCCAAGCTGAAGTCATTCTCCCCAGAAATAACAAAAGTTGGAAG |
| wings) | AGAAGCCGGAGACAGGATAGGTGCAGGAAGCCCACACTTTGAGGGCAGCACTCAGACACCCTCTCCTGTG |
| SEQ ID | TGCAGGACGTGCCGAATGTTCAGGTGCAATGAGAATGAGCCATGCttggcttataaggtacgactgtgcc |
| NO: 1123 | ttctagttgccagccatctgttgtttgcccctcccccgtgccttccttgaccctggaaggtgccactccc |
| | actgtcctttcctaataaaatgaggaaattgcatcgcattgtctgagtaggtgtcattctattctggggg |
| | gtggggtggggcaggacagcaagggggaggattgggaagacaatagcaggcatgctggggatgcggtggg |
| | ctctatgggataagcttgatatcgaattcatcgatgttaataattaacatatatgttaatcattaacata |
| | tagttaattattaaccgctatgttaatgattaacaacggttaataattaacatatatgttaatcattaac |
| | atataactagtctagagggtatataatgggggccactagtctactaccagagTtcatcgctagcgctacc |
| | ggatccgccaccATGGCCCTGCCAGTAACGGCTCTGCTGCTGCCACTTGCTCTGCTCCTCCATGCAGCCA |
| | GGCCTCAAGTGCAGCTGGTCCAGTCTGGGGCGGAAGTGAAAAAGCCCGGAGCTAGTGTAAAGGTGTCCTG |
| | TAAAGCCAGCGGCTACACCTTCACCGGTTATTACCTGCATTGGGTCCGGCAGGCTCCTGGCCAGGGCCTG |
| | GAGTGGATGGGCTGGATTTCCGCATATAACGGAAACACAAATTACGCCCAGAACCTGCAAGGCCGCGTGA |
| | CCATGACCAGGGACACAAGCACTAGCACTGTCTACATGGAGTTGTCTAGCTTGAGAAGCGAAGATACCGC |
| | TGTGTACTATTGCGCCCGACACTCTTACTCGGGCTCATACTCAACGCTACCCTATTATGGGATGGATGTT |
| | TGGGGTCAAGGGACAACGGTCACAGTATCCTCTGGAGGCGGTGGCAGCGGAGGAGGCGGGTCTGGAGGTG |
| | GTGGATCAGACATTCAGATGACCCAGTCACCAAGTTCCTTATCCGCAAGCGTTGGGGATCGTGTTACAAT |
| | TACTTGCAGGGCCTCGCAAGGGATCTCTAATTATCTCGCTTGGTACCAGCAGAAACCTGGGAAAGCACCC |
| | AAGCTGCTGATCTACACTGCAAGCACACTTTTTCCAGGAGTGCCGTCAAGATTCTCTGGGTCCGGGAGTG |
| | GCACTGACTTCACCCTTACCATCTCCTCCCTCCAGCCTGAGGACTTTGCCACATATTATTGTCAACAGAG |
| | TTACTCCATACCACTCACGTTTGGCGGCGGAACAAAaGTtGAAATCAAGGCGGCAGCAaccacgacgcca |
| | gcgccgcgaccaccaacaccggcgcccaccatcgcgtcgcagccactgtcactgcgcccagaagcgtgcc |
| | ggccagcggcggggggcgcagtgcaTacgagggggctggacttcgcctgtgatatctacatctgggcgcc |
| | cttggccgggacttgtggggtccttctcctgtcactggttatcaccctttactgcaaacggggcagaaag |
| | aaactcctgtatatattcaaacaaccatttatgagaccagtacaaactactcaagaagaggacggctgta |
| | gctgccgatttccagaagaagaagaaggaggatgtgaactgagagtgaagttcagcaggagcgcagacgc |
| | ccccgcgtaccagcagggccagaaccagctctataacgagctcaatctaggacgaagagaggagtacgat |
| | gttttggacaagaggcgtggccgggaccctgagatggggggaaagccgagaaggaagaaccctcaggaag |
| | gcctgtacaatgaactgcagaaagataagatggcggaggcctacagtgagattgggatgaaaggcgagcg |
| | ccggaggggcaaggggcacgatggcctttaccagggtctcagtacagccaccaaggacacctacgacgcc |
| | cttcacatgcaggccctgccccctaggtaaaatcaacctctggattacaaaatttgtgaaagattgactg |
| | gtattcttaactatgttgctccttttacgctatgtggatacgctgctttaatgcctttgtatcatgctat |
| | tgcttcccgtatggctttcattttctcctccttgtataaatcctggttgctgtctctttatgaggagttg |
| | tggcccgttgtcaggcaacgtggcgtggtCtgcactgtgtttgctgacgcaacccccactggttggggca |
| | ttgccaccacctgtcagctcctttccgggactttcgctttccccctccctattgccacggcggaactcat |
| | cgccgcctgccttgcccgctgctggacaggggctcggctgttgggcactgacaattccgtggtgttgtcg |
| | gggaaatcatcgtcctttccttggctgctcgcctgtgttgccacctggattctgcgcgggacgtccttct |
| | gctacgtcccttcggccctcaatccagcggaccttccttcccgcggcctgctgccggctctgcggcctct |
| | tccgcgtcttcgccttcgccctcagacgagtcggatctccctttgggccgcctccccgcctggatccttg |
| | acttgcggccaacttgtttattgcagcttataatggttacaaataaagcaatagcatcacaaatttcaca |
| | aataaagcatttttttcactgcattctagttgtggtttgtccaaactcatcaatgtatcttatcatgtct |
| | gggatccttgacttgcggccgcaactcccacctgcaacatgcgtgactgactgaggccgcgactctagag |
| | tcgaccggatctgcgatcgctccggtgcccgtcagtgggcagagcgcacatcgcccacagtccccgagaa |
| | gttggggggaggggtcggcaattgaacgggtgcctagagaaggtggcgcggggtaaactgggaaagtgat |
| | gtcgtgtactggctccgcctttttcccgagggtgggggagaaccgtatataagtgcagtagtcgccgtga |
| | acgttctttttcgcaacgggtttgccgccagaacacagctgaagcttcgaggggctcgcatctctccttc |
| | acgcgcccgccgccctacctgaggccgccatccacgccggttgagtcgcgttctgccgcctcccgcctgt |
| | ggtgcctcctgaactgcgtccgccgtctaggtaaGTcgactcgttggatccCCACTACCCGGATCAACGC |
| | CCTAGGTTTATGTTTGGATGAACTGACATACGCGTATCCGTCTTAAGAATCTTTTCAAACACTAGTAGTG |
| | AAATATATATTAAACTAGTGTTTGAAAAGATTCTTATTACGGTAACGCGGAATTCGCAACTATTTTATCA |
| | ATTTTTTGCGTCGACACTTCAAGGGGCTTGCGGCCGCAACCATCTCCATGGCTGTTTGAATGAGGCTTCA |
| | GTACTTTACAGAATCGTTGCCTGCACATCTTGGAAACACTTGCTGGGATTACTTCGACTTCTTAACCCAA |
| | CAGAAGGCTCGAGAAGGTATATTGCTGTTGACAGTGAGCGCCAGTGTGAAGCTCTTGTCAGATAGTGAAG |
| | CCACAGATGTATCTGACAAGAGCTTCACACTGATGCCTACTGCCTCGGACTTCAAGGGGCTAGAATTCGA |
| | GCAATTATCTTGTTTACTAAAACTGAATACCTTGCTATCTCTTTGATACATTTTTACAAAGCTGAATTAA |
| | AATGGTATAAATTAAATCACTTTGACGGTGACTTAGGAGTATGCCGGATCAACGCCCTAGGTTTATGTTT |
| | GGATGAACTGACATACGCGTATCCGTCTAAAAATCAATCTCATTTCCTGGTAGTGAAATATATATTAAAC |
| | CAGGAAATGAGATTGATTTTTTTACGGTAACGCGGAATTCGCAACTATTTTATCAATTTTTTGCGTCGAC |
| | GACTGTGACAGCAGAGTATGCCGGATCAACGCCCTAGGTTTATGTTTGGATGAACTGACATACGCGTATC |
| | CGTCTTATGTAAAAGACAAACAATGCGTAGTGAAATATATATTAAACGCATTGTTTGTCTTTTACATATT |
| | ACGGTAACGCGGAATTCGCAACTATTTTATCAATTTTTTGCGTCGACCGGAACTATCTTGAAGAGTAGTA |
| | GTGGactagtgtgacgctgctgacccctttctttcccttctACAGATCCAAGCTGTGACCGGCGCCTACa |
| | cctgcagcccaagcttaccatggccttaccagtgaccgccttgctcctgccgctggccttgctgctccac |
| | gccgccaggcctGAGATAGTCCTGACCCAGTCACCCGATTTCCAGAGTGTTACTCCTAAGGAGAAAGTGA |
| | CTATAACATGCCGGGCATCTCAGTCTGTCGGAAGCGGGCTACATTGGTACCAGCAGAAGCCTGACCAGAG |
| | CCCGAAACTGCTCATCAAATATGCCTCCCAGTCGTTTTCTGGCGTGCCCTCTCGCTTTTCCGGAAGCGGA |
| | TCTGGCACAGACTTCACCTTGACCATCAATAGCCTGGAAACTGAGGACGCCGCTACGTATTTCTGCCAGC |
| | AGTCCTCCAGTCTGCCTTGGACATTTGGTCAGGGAACGAAGGTGGAGATCAAGGGTTCAACATCAGGGAG |
| | CGGGAAACCGGGCTCTGGCGAGGGCTCAACAAAGGGACAAGTGCAACTGCAAGAATCGGGACCCGGGCTG |
| | GTTAAACCAAGTGAAACCCTTTCCCTCACTTGTACCGTAAGCGGCGGTAGCGTGTCCTCTGGTAGCTACT |
| | ATTGGAGTTGGATTAGGCAGGCGCCAGGGAAAGGCCTCGAATGGATTGGGTATATCTACTACAGCGGCAG |
| | TAACTACTACAACCCATCATTGAAGTCTAGAGTGACAATTAGTGTCGATACCTCTAAAAATCAATTCTCA |
| | CTTAAGCTGCGAGCTGTAACCGCCGCAGACACTGCGGTGTACTATTGTGCCCGTTGGATGACCACTATCA |
| | AGGGCTACTTCGATTATTGGGGACAAGGGACATTAGTTACGGTGTCCTCCGCAACCAcgacgccagcgcc |
| | gcgaccaccaacaccggcgcccaccatcgcgtcgcagcccctgtccctgcgcccTgaggcgtgcttcatg |
| | tacgtggcggcggccgcctttgtgcttctgttcttcgtgggctgcggggtgctgctgtcccgtaaacgCa |
| | gacgtcaacacggtcaactgtggtttccagaaggttttaaggtctccgaagcaagtaagaagaaaagacg |
| | tgaaccactgggagaagatagcgtcggtctgaaaccactcaagaatgccatggtttctaaactgagccag |
| | ctgcagacggagctcctggcggccctgctggagtcagggctgagcaaagaggcactgctccaggcactgg |
| | gCgagccggggccctacctcctggctggagaaggccccctggacaagggggagtcctgcggcggcggtcg |
| | aggggagctggctgagctgcccaatgggctgggggagactcggggctccgaggacgagacCgacgacgat |
| | ggggaagacttcacgccacccatcctcaaagagctggagaacctcagccctgaggaggcggcccaccaga |
| | aagccgtggtggagacccttctgcaggaggacccgtggcgtgtggcgaagatggtcaagtcctacctgca |
| | gcagcacaacatcccacagcgggaggtggtcgataccactggcctcaaccagtcccacctgtcccaacac |
| | ctcaacaagggcactcccatgaagacgcagaagcgggccgccctgtacacctggtaTgtccgcaagcagc |
| | gagaggtggcgcagcagttcacccatgcagggcagggagggctgattgaAgaGcccacaggAgatgagct |
| | accaaccaagaaggggcggaggaaccgtttcaagtggggcccagcatcccagcagatcctgttccaggcc |
| | tatgagaggcagaagaaccctagcaaggaggagcgagaAacgctagtggaggagtgcaatagggcggaat |
| | gcatccagagaggTgtgtcAccatcacaAgcacaAggTctgggctccaacctcgtcacggaggtgcgtgt |
| | ctacaactggtttgccaaccggcgcaaagaagaagccttccggcacaagctggccatgacctgcagggat |
| | gagtttcccaccatggtgtttccttctgggcagatcagccaggcctcggccttggccccggcccctcccc |
| | aagtcctgccccaggctccagcccctgcccctgctccagccatggtatcagctctggcccaggccccagc |
| | ccctgtcccagtcctagccccaggccctcctcaAgctgtggccccacctgcccccaagcccacccaAgct |
| | ggggaaggaacgctgtcagaggccctgctgcagctgcagtttgatgatgaagacctgggggccttgcttg |
| | gcaacagcacagacccagctgtgttcacagacctggcatccgtcgacaactccgagtttcagcagctgct |
| | gaaccagggcatacctgtggccccccacacaactgagcccatgctgatggagtaccctgaggctataact |
| | cgcctagtgacaggggcccagaggccccccgacccagctcctgctccactgggggccccggggctcccca |
| | atggcctcctttcaggagatgaagacttctcctccattgcggacatggacttctcagccctgctgagtca |
| | gatcagctccTAAAGGACGGGTGGCATCCCTGTGACCCCTCCCCAGTGCCTCTCCTGGCCCTGGAAGTTG |
| | CCACTCCAGTGCCCACCAGCCTTGTCCTAATAAAATTAAGTTGCATCATTTTGTCTGACTAGGTGTCCTT |
| | CTATAATATTATGGGGTGGAGGGGGGTGGTATGGAGCAAGGGGCAAGTTGGGAAGACAACCTGTAGGGCC |
| | TGCGGGGTCTATTGGGAACCAAGCTGGAGTGCAGTGGCACAATCTTGGCTCACTGCAATCTCCGCCTCCT |
| | GGGTTCAAGCGATTCTCCTGCCTCAGCCTCCCGAGTTGTTGGGATTCCAGGCATGCATGACCAGGCTCAG |
| | CTAATTTTTGTTTTTTTGGTAGAaACGGGGTTTCACCATATTGGCCAGGCTGGTCTCCAACTCCTAATCT |
| | CAGGTGATCTACCCACCTTGGCCTCCCAAATTGCTGGGATTACAGGCGTGAACCACTGCTCCCttccctg |
| | tccttcCGAGGGCAATCTGGCCCATCAAGTGGCCTTCGCCTCTGGGAGTAACAAAAATGCACTTCAAAAT |
| | AGCTTCTGTAATCAAGCTGCATGGGTGGAGTACTCCCCAGCTGACTCCAGGAAGTTCTCTATCCAAAGCT |
| | ATTCATTAGGCCAGAGCTGTGCAAATAATTAGTCACCCACTTGCTCCATAACCCTCCATGACAGCCCAGG |
| | CATTGAGTCCAGGTGGGACCATCAAGCCATGCTCTGGTGGCTCATGCATTATCATAGAAATGGGAGGCTT |
| | TATTTATTTTACTAAAAAGAACAAAAACAACAGACTGCTGTCCTTTAGACAATAGGATCACGTCATCTGA |
| | GCCCTCTGTGCCCCAGGTGACAAGCCCAGCCCCAAGTTCTCTTTCCTCAGCCTCCCCACACATGTTCTGG |
| | AGGAGATGGGCCCAGCAGGCTGCTCTGAGGCCTGGCCCCTCGTAAGCCAAGCATGGCTC |
| LG 47 | GAGCCATGCTTGGCTTACGAGGGCGACCAACCCATCAAACTCCCCGCCCCCAGCACTTTTATTTCTCCTC |
| (with | TTTAGGAAGTACACTTCAGTATCTTTGGCACAGTGCATGAGCACGACTAAAGTAAAACATCGCAGAAAAC |
| sgRNA | ATAGCTTTAGTCTACCCTTCGTGTCCTAAAAGGAAAACCAGTAGCTTCCCAGGCCACCGGAAGGGCAACA |
| homology | CATGTCCTCTGCAGTTTCTGCACACGGGAAGGTAAAGACAGAGAGAGGACCTACTCCTCAACACAGAAAC |
| wings) | AGAAGCCGGAGACAGGATAGGTGCAGGAAGCCCACACTTTGAGGGCAGCACTCAGACACCCTCTCCTGTG |
| and | ATTTCAAAATCTTTCCTCGCCTGCAACCCAAGCTGAAGTCATTCTCCCCAGAAATAACAAAAGTTGGAAG |
| NO: 1124 | ttctagttgccagccatctgttgtttgcccctcccccgtgccttccttgaccctggaaggtgccactccc |
| SEQ ID | TGCAGGACGTGCCGAATGTTCAGGTGCAATGAGAATGAGCCATGCttggcttataaggtacgactgtgcc |
| | actgtcctttcctaataaaatgaggaaattgcatcgcattgtctgagtaggtgtcattctattctggggg |
| | gtggggtggggcaggacagcaagggggaggattgggaagacaatagcaggcatgctggggatgcggtggg |
| | ctctatgggataagcttgatatcgaattcatcgatgttaataattaacatatatgttaatcattaacata |
| | tagttaattattaaccgctatgttaatgattaacaacggttaataattaacatatatgttaatcattaac |
| | atataactagtctagagggtatataatgggggccactagtctactaccagagTtcatcgctagcgctacc |
| | ggatccgccaccATGGCCCTGCCAGTAACGGCTCTGCTGCTGCCACTTGCTCTGCTCCTCCATGCAGCCA |
| | GGCCTCAGGTCCAGTTGGTACAGAGCGGCGCCGAAGTGAAAAAGCCTGGGGCGTCCGTCAAAGTGTCTTG |
| | CAAGGCCTCCGGCTATACATTCACCGGGTACTACATGCATTGGGTGCGGCAGGCACCTGGCCAGGGTCTA |
| | GAATGGATGGGCCGGATCAATCCCAACTCCGGCGGCACAAACTATGCTCAGAAATTTCAAGGTCGCGTCA |
| | CCATGACCCGTGACACAAGTACGAGCACCGTCTACATGGAGCTGTCCTCCCTCAGGAGCGAGGATACAGC |
| | CGTGTACTATTGTGCAAGGGAGCGCGCCGGCTATAGCAGCGGGCAGTTCGATTATTGGGGACAAGGGACT |
| | CTGGTAACTGTGTCCTCCGGAGGCGGAGGATCAGGCGGAGGAGGCTCAGGAGGTGGAGGTTCTGACATTC |
| | AGATGACTCAATCTCCCTCGTCACTGTCAGCTAGTGTTGGGGATAGAGTGACTATTACCTGCCGAGCCAG |
| | TCAGTCAATATCTAACTGGCTCGCATGGTACCAGCAGAAGCCAGGGAAGGCTCCCAAACTGCTGATCTAC |
| | GCCGCGAGCACCCTTCAGAATGGCGTGCCGTCTAGATTTAGCGGTTCTGGGTCTGGGACCGACTTTACAC |
| | TTACTATCAGTAGTTTACAACCAGAGGACTTTGCTACTTATTACTGTCAACAGAGCTACACCTTCCCTAT |
| | TACGTTCGGCCAGGGAACAAAAGTTGAAATCAAGGCGGCAGCAaccacgacgccagcgccgcgaccacca |
| | acaccggcgcccaccatcgcgtcgcagccactgtcactgcgcccagaagcgtgccggccagcggcggggg |
| | gcgcagtgcaTacgagggggctggacttcgcctgtgatatctacatctgggcgcccttggccgggacttg |
| | tggggtccttctcctgtcactggttatcaccctttactgcaaacggggcagaaagaaactcctgtatata |
| | ttcaaacaaccatttatgagaccagtacaaactactcaagaagaggacggctgtagctgccgatttccag |
| | aagaagaagaaggaggatgtgaactgagagtgaagttcagcaggagcgcagacgcccccgcgtaccagca |
| | gggccagaaccagctctataacgagctcaatctaggacgaagagaggagtacgatgttttggacaagagg |
| | cgtggccgggaccctgagatggggggaaagccgagaaggaagaaccctcaggaaggcctgtacaatgaac |
| | tgcagaaagataagatggcggaggcctacagtgagattgggatgaaaggcgagcgccggaggggcaaggg |
| | gcacgatggcctttaccagggtctcagtacagccaccaaggacacctacgacgcccttcacatgcaggcc |
| | ctgccccctaggtaaaatcaacctctggattacaaaatttgtgaaagattgactggtattcttaactatg |
| | ttgctccttttacgctatgtggatacgctgctttaatgcctttgtatcatgctattgcttcccgtatggc |
| | tttcattttctcctccttgtataaatcctggttgctgtctctttatgaggagttgtggcccgttgtcagg |
| | caacgtggcgtggtCtgcactgtgtttgctgacgcaacccccactggttggggcattgccaccacctgtc |
| | agctcctttccgggactttcgctttccccctccctattgccacggcggaactcatcgccgcctgccttgc |
| | ccgctgctggacaggggctcggctgttgggcactgacaattccgtggtgttgtcggggaaatcatcgtcc |
| | tttccttggctgctcgcctgtgttgccacctggattctgcgcgggacgtccttctgctacgtcccttcgg |
| | ccctcaatccagcggaccttccttcccgcggcctgctgccggctctgcggcctcttccgcgtcttcgcct |
| | tcgccctcagacgagtcggatctccctttgggccgcctccccgcctggatccttgacttgcggccaactt |
| | gtttattgcagcttataatggttacaaataaagcaatagcatcacaaatttcacaaataaagcatttttt |
| | tcactgcattctagttgtggtttgtccaaactcatcaatgtatcttatcatgtctgggatccttgacttg |
| | cggccgcaactcccacctgcaacatgcgtgactgactgaggccgcgactctagagtcgaccggatctgcg |
| | atcgctccggtgcccgtcagtgggcagagcgcacatcgcccacagtccccgagaagttggggggaggggt |
| | cggcaattgaacgggtgcctagagaaggtggcgcggggtaaactgggaaagtgatgtcgtgtactggctc |
| | cgcctttttcccgagggtgggggagaaccgtatataagtgcagtagtcgccgtgaacgttctttttcgca |
| | acgggtttgccgccagaacacagctgaagcttcgaggggctcgcatctctccttcacgcgcccgccgccc |
| | tacctgaggccgccatccacgccggttgagtcgcgttctgccgcctcccgcctgtggtgcctcctgaact |
| | gcgtccgccgtctaggtaaGTcgactcgttggatccCCACTACCCGGATCAACGCCCTAGGTTTATGTTT |
| | GGATGAACTGACATACGCGTATCCGTCTTAAGAATCTTTTCAAACACTAGTAGTGAAATATATATTAAAC |
| | TAGTGTTTGAAAAGATTCTTATTACGGTAACGCGGAATTCGCAACTATTTTATCAATTTTTTGCGTCGAC |
| | ACTTCAAGGGGCTTGCGGCCGCAACCATCTCCATGGCTGTTTGAATGAGGCTTCAGTACTTTACAGAATC |
| | GTTGCCTGCACATCTTGGAAACACTTGCTGGGATTACTTCGACTTCTTAACCCAACAGAAGGCTCGAGAA |
| | GGTATATTGCTGTTGACAGTGAGCGCCAGTGTGAAGCTCTTGTCAGATAGTGAAGCCACAGATGTATCTG |
| | ACAAGAGCTTCACACTGATGCCTACTGCCTCGGACTTCAAGGGGCTAGAATTCGAGCAATTATCTTGTTT |
| | ACTAAAACTGAATACCTTGCTATCTCTTTGATACATTTTTACAAAGCTGAATTAAAATGGTATAAATTAA |
| | ATCACTTTGACGGTGACTTAGGAGTATGCCGGATCAACGCCCTAGGTTTATGTTTGGATGAACTGACATA |
| | CGCGTATCCGTCTAAAAATCAATCTCATTTCCTGGTAGTGAAATATATATTAAACCAGGAAATGAGATTG |
| | ATTTTTTTACGGTAACGCGGAATTCGCAACTATTTTATCAATTTTTTGCGTCGACGACTGTGACAGCAGA |
| | GTATGCCGGATCAACGCCCTAGGTTTATGTTTGGATGAACTGACATACGCGTATCCGTCTTATGTAAAAG |
| | ACAAACAATGCGTAGTGAAATATATATTAAACGCATTGTTTGTCTTTTACATATTACGGTAACGCGGAAT |
| | TCGCAACTATTTTATCAATTTTTTGCGTCGACCGGAACTATCTTGAAGAGTAGTAGTGGactagtgtgac |
| | gctgctgacccctttctttcccttctACAGATCCAAGCTGTGACCGGCGCCTACacctgcagcccaagct |
| | taccatggccttaccagtgaccgccttgctcctgccgctggccttgctgctccacgccgccaggcctGAG |
| | ATAGTCCTGACCCAGTCACCCGATTTCCAGAGTGTTACTCCTAAGGAGAAAGTGACTATAACATGCCGGG |
| | CATCTCAGTCTGTCGGAAGCGGGCTACATTGGTACCAGCAGAAGCCTGACCAGAGCCCGAAACTGCTCAT |
| | CAAATATGCCTCCCAGTCGTTTTCTGGCGTGCCCTCTCGCTTTTCCGGAAGCGGATCTGGCACAGACTTC |
| | ACCTTGACCATCAATAGCCTGGAAACTGAGGACGCCGCTACGTATTTCTGCCAGCAGTCCTCCAGTCTGC |
| | CTTGGACATTTGGTCAGGGAACGAAGGTGGAGATCAAGGGTTCAACATCAGGGAGCGGGAAACCGGGCTC |
| | TGGCGAGGGCTCAACAAAGGGACAAGTGCAACTGCAAGAATCGGGACCCGGGCTGGTTAAACCAAGTGAA |
| | ACCCTTTCCCTCACTTGTACCGTAAGCGGCGGTAGCGTGTCCTCTGGTAGCTACTATTGGAGTTGGATTA |
| | GGCAGGCGCCAGGGAAAGGCCTCGAATGGATTGGGTATATCTACTACAGCGGCAGTAACTACTACAACCC |
| | ATCATTGAAGTCTAGAGTGACAATTAGTGTCGATACCTCTAAAAATCAATTCTCACTTAAGCTGCGAGCT |
| | GTAACCGCCGCAGACACTGCGGTGTACTATTGTGCCCGTTGGATGACCACTATCAAGGGCTACTTCGATT |
| | ATTGGGGACAAGGGACATTAGTTACGGTGTCCTCCGCAACCAcgacgccagcgccgcgaccaccaacacc |
| | ggcgcccaccatcgcgtcgcagcccctgtccctgcgcccTgaggcgtgcttcatgtacgtggcggcggcc |
| | gcctttgtgcttctgttcttcgtgggctgcggggtgctgctgtcccgtaaacgCagacgtcaacacggtc |
| | aactgtggtttccagaaggttttaaggtctccgaagcaagtaagaagaaaagacgtgaaccactgggaga |
| | agatagcgtcggtctgaaaccactcaagaatgccatggtttctaaactgagccagctgcagacggagctc |
| | ctggcggccctgctggagtcagggctgagcaaagaggcactgctccaggcactgggCgagccggggccct |
| | acctcctggctggagaaggccccctggacaagggggagtcctgcggcggcggtcgaggggagctggctga |
| | gctgcccaatgggctgggggagactcggggctccgaggacgagacCgacgacgatggggaagacttcacg |
| | ccacccatcctcaaagagctggagaacctcagccctgaggaggcggcccaccagaaagccgtggtggaga |
| | cccttctgcaggaggacccgtggcgtgtggcgaagatggtcaagtcctacctgcagcagcacaacatccc |
| | acagcgggaggtggtcgataccactggcctcaaccagtcccacctgtcccaacacctcaacaagggcact |
| | cccatgaagacgcagaagcgggccgccctgtacacctggtaTgtccgcaagcagcgagaggtggcgcagc |
| | agttcacccatgcagggcagggagggctgattgaAgaGcccacaggAgatgagctaccaaccaagaaggg |
| | gcggaggaaccgtttcaagtggggcccagcatcccagcagatcctgttccaggcctatgagaggcagaag |
| | aaccctagcaaggaggagcgagaAacgctagtggaggagtgcaatagggcggaatgcatccagagaggTg |
| | tgtcAccatcacaAgcacaAggTctgggctccaacctcgtcacggaggtgcgtgtctacaactggtttgc |
| | caaccggcgcaaagaagaagccttccggcacaagctggccatgacctgcagggatgagtttcccaccatg |
| | gtgtttccttctgggcagatcagccaggcctcggccttggccccggcccctccccaagtcctgccccagg |
| | ctccagcccctgcccctgctccagccatggtatcagctctggcccaggccccagcccctgtcccagtcct |
| | agccccaggccctcctcaAgctgtggccccacctgcccccaagcccacccaAgctggggaaggaacgctg |
| | tcagaggccctgctgcagctgcagtttgatgatgaagacctgggggccttgcttggcaacagcacagacc |
| | cagctgtgttcacagacctggcatccgtcgacaactccgagtttcagcagctgctgaaccagggcatacc |
| | tgtggccccccacacaactgagcccatgctgatggagtaccctgaggctataactcgcctagtgacaggg |
| | gcccagaggccccccgacccagctcctgctccactgggggccccggggctccccaatggcctcctttcag |
| | gagatgaagacttctcctccattgcggacatggacttctcagccctgctgagtcagatcagctccTAAAG |
| | GACGGGTGGCATCCCTGTGACCCCTCCCCAGTGCCTCTCCTGGCCCTGGAAGTTGCCACTCCAGTGCCCA |
| | CCAGCCTTGTCCTAATAAAATTAAGTTGCATCATTTTGTCTGACTAGGTGTCCTTCTATAATATTATGGG |
| | GTGGAGGGGGGTGGTATGGAGCAAGGGGCAAGTTGGGAAGACAACCTGTAGGGCCTGCGGGGTCTATTGG |
| | GAACCAAGCTGGAGTGCAGTGGCACAATCTTGGCTCACTGCAATCTCCGCCTCCTGGGTTCAAGCGATTC |
| | TCCTGCCTCAGCCTCCCGAGTTGTTGGGATTCCAGGCATGCATGACCAGGCTCAGCTAATTTTTGTTTTT |
| | TTGGTAGAaACGGGGTTTCACCATATTGGCCAGGCTGGTCTCCAACTCCTAATCTCAGGTGATCTACCCA |
| | CCTTGGCCTCCCAAATTGCTGGGATTACAGGCGTGAACCACTGCTCCCttccctqtccttcCGAGGGCAA |
| | TCTGGCCCATCAAGTGGCCTTCGCCTCTGGGAGTAACAAAAATGCACTTCAAAATAGCTTCTGTAATCAA |
| | GCTGCATGGGTGGAGTACTCCCCAGCTGACTCCAGGAAGTTCTCTATCCAAAGCTATTCATTAGGCCAGA |
| | GCTGTGCAAATAATTAGTCACCCACTTGCTCCATAACCCTCCATGACAGCCCAGGCATTGAGTCCAGGTG |
| | GGACCATCAAGCCATGCTCTGGTGGCTCATGCATTATCATAGAAATGGGAGGCTTTATTTATTTTACTAA |
| | AAAGAACAAAAACAACAGACTGCTGTCCTTTAGACAATAGGATCACGTCATCTGAGCCCTCTGTGCCCCA |
| | GGTGACAAGCCCAGCCCCAAGTTCTCTTTCCTCAGCCTCCCCACACATGTTCTGGAGGAGATGGGCCCAG |
| | CAGGCTGCTCTGAGGCCTGGCCCCTCGTAAGCCAAGCATGGCTC |
| 5′ | CGACCAACCCATCAAACTCCCCGCCCCCAGCACTTTTATTTCTCCTCTTTAGGAAGTACACTTCAGTATC |
| homology | TTTGGCACAGTGCATGAGCACGACTAAAGTAAAACATCGCAGAAAACATAGCTTTAGTCTACCCTTCGTG |
| wing | TCCTAAAAGGAAAACCAGTAGCTTCCCAGGCCACCGGAAGGGCAACACATGTCCTCTGCAGTTTCTGCAC |
| SEQ ID | ACGGGAAGGTAAAGACAGAGAGAGGACCTACTCCTCAACACAGAAACATTTCAAAATCTTTCCTCGCCTG |
| NO: 1235 | CAACCCAAGCTGAAGTCATTCTCCCCAGAAATAACAAAAGTTGGAAGAGAAGCCGGAGACAGGATAGGTG |
| | CAGGAAGCCCACACTTTGAGGGCAGCACTCAGACACCCTCTCCTGTGTGCAGGACGTGCCGAATGTTCAG |
| | GTGCAATGAGAATGAGCCATGCTTGGCTTA |
| 5′ | CGAGGGCAATCTGGCCCATCAAGTGGCCTTCGCCTCTGGGAGTAACAAAAATGCACTTCAAAATAGCTTC |
| homology | TGTAATCAAGCTGCATGGGTGGAGTACTCCCCAGCTGACTCCAGGAAGTTCTCTATCCAAAGCTATTCAT |
| wing | TAGGCCAGAGCTGTGCAAATAATTAGTCACCCACTTGCTCCATAACCCTCCATGACAGCCCAGGCATTGA |
| SEQ ID | GTCCAGGTGGGACCATCAAGCCATGCTCTGGTGGCTCATGCATTATCATAGAAATGGGAGGCTTTATTTA |
| NO: 1236 | TTTTACTAAAAAGAACAAAAACAACAGACTGCTGTCCTTTAGACAATAGGATCACGTCATCTGAGCCCTC |
| | TGTGCCCCAGGTGACAAGCCCAGCCCCAAGTTCTCTTTCCTCAGCCTCCCCACACATGTTCTGGAGGAGA |
| | TGGGCCCAGCAGGCTGCTCTGAGGCCTGGC |
| sgRNA 94 | GAGCCATGCTTGGCTTACGA |
| sequence | |
| SEQ ID | |
| NO: 932 | |
| sgRNA 94 | TCGTAAGCCAAGCATGGCTC |
| reverse | |
| complement | |
| sequence | |
| SEQ ID | |
| NO: 1237 | |
| LG 219 | cgactgtgccttctagttgccagccatctgttgtttgcccctcccccgtgccttccttgaccctggaagg |
| core | tgccactcccactgtcctttcctaataaaatgaggaaattgcatcgcattgtctgagtaggtgtcattct |
| insert | attctggggggtggggtggggcaggacagcaagggggaggattgggaagacaatagcaggcatgctgggg |
| (w/o | atgcggtgggctctatgggataagcttgatatcgaattcatcgatgttaataattaacatatatgttaat |
| homology | cattaacatatagttaattattaaccgctatgttaatgattaacaacggttaataattaacatatatgtt |
| wings) | aatcattaacatataactagtctagagggtatataatgggggccactagtctactaccagagTtcatcgc |
| SEQ ID | tagcgctaccggatccgccaccATGGCCCTGCCAGTAACGGCTCTGCTGCTGCCACTTGCTCTGCTCCTC |
| NO: 1238 | CATGCAGCCAGGCCTCAAGTGCAGCTGGTCCAGTCTGGGGCGGAAGTGAAAAAGCCCGGAGCTAGTGTAA |
| | AGGTGTCCTGTAAAGCCAGCGGCTACACCTTCACCGGTTATTACCTGCATTGGGTCCGGCAGGCTCCTGG |
| | CCAGGGCCTGGAGTGGATGGGCTGGATTTCCGCATATAACGGAAACACAAATTACGCCCAGAACCTGCAA |
| | GGCCGCGTGACCATGACCAGGGACACAAGCACTAGCACTGTCTACATGGAGTTGTCTAGCTTGAGAAGCG |
| | AAGATACCGCTGTGTACTATTGCGCCCGACACTCTTACTCGGGCTCATACTCAACGCTACCCTATTATGG |
| | GATGGATGTTTGGGGTCAAGGGACAACGGTCACAGTATCCTCTGGAGGCGGTGGCAGCGGAGGAGGCGGG |
| | TCTGGAGGTGGTGGATCAGACATTCAGATGACCCAGTCACCAAGTTCCTTATCCGCAAGCGTTGGGGATC |
| | GTGTTACAATTACTTGCAGGGCCTCGCAAGGGATCTCTAATTATCTCGCTTGGTACCAGCAGAAACCTGG |
| | GAAAGCACCCAAGCTGCTGATCTACACTGCAAGCACACTTTTTCCAGGAGTGCCGTCAAGATTCTCTGGG |
| | TCCGGGAGTGGCACTGACTTCACCCTTACCATCTCCTCCCTCCAGCCTGAGGACTTTGCCACATATTATT |
| | GTCAACAGAGTTACTCCATACCACTCACGTTTGGCGGCGGAACAAAaGTtGAAATCAAGGCGGCAGCAac |
| | cacgacgccagcgccgcgaccaccaacaccggcgcccaccatcgcgtcgcagccactgtcactgcgccca |
| | gaagcgtgccggccagcggcggggggcgcagtgcaTacgagggggctggacttcgcctgtgatatctaca |
| | tctgggcgcccttggccgggacttgtggggtccttctcctgtcactggttatcaccctttactgcaaacg |
| | gggcagaaagaaactcctgtatatattcaaacaaccatttatgagaccagtacaaactactcaagaagag |
| | gacggctgtagctgccgatttccagaagaagaagaaggaggatgtgaactgagagtgaagttcagcagga |
| | gcgcagacgcccccgcgtaccagcagggccagaaccagctctataacgagctcaatctaggacgaagaga |
| | ggagtacgatgttttggacaagaggcgtggccgggaccctgagatggggggaaagccgagaaggaagaac |
| | cctcaggaaggcctgtacaatgaactgcagaaagataagatggcggaggcctacagtgagattgggatga |
| | aaggcgagcgccggaggggcaaggggcacgatggcctttaccagggtctcagtacagccaccaaggacac |
| | ctacgacgcccttcacatgcaggccctgccccctaggtaaaatcaacctctggattacaaaatttgtgaa |
| | agattgactggtattcttaactatgttgctccttttacgctatgtggatacgctgctttaatgcctttgt |
| | atcatgctattgcttcccgtatggctttcattttctcctccttgtataaatcctggttgctgtctcttta |
| | tgaggagttgtggcccgttgtcaggcaacgtggcgtggtCtgcactgtgtttgctgacgcaacccccact |
| | ggttggggcattgccaccacctgtcagctcctttccgggactttcgctttccccctccctattgccacgg |
| | cggaactcatcgccgcctgccttgcccgctgctggacaggggctcggctgttgggcactgacaattccgt |
| | ggtgttgtcggggaaatcatcgtcctttccttggctgctcgcctgtgttgccacctggattctgcgcggg |
| | acgtccttctgctacgtcccttcggccctcaatccagcggaccttccttcccgcggcctgctgccggctc |
| | tgcggcctcttccgcgtcttcgccttcgccctcagacgagtcggatctccctttgggccgcctccccgcc |
| | tggatccttgacttgcggccaacttgtttattgcagcttataatggttacaaataaagcaatagcatcac |
| | aaatttcacaaataaagcatttttttcactgcattctagttgtggtttgtccaaactcatcaatgtatct |
| | tatcatgtctgggatccttgacttgcggccgcaactcccacctgcaacatgcgtgactgactgaggccgc |
| | gactctagagtcgaccggatctgcgatcgctccggtgcccgtcagtgggcagagcgcacatcgcccacag |
| | tccccgagaagttggggggaggggtcggcaattgaacgggtgcctagagaaggtggcgcggggtaaactg |
| | ggaaagtgatgtcgtgtactggctccgcctttttcccgagggtgggggagaaccgtatataagtgcagta |
| | gtcgccgtgaacgttctttttcgcaacgggtttgccgccagaacacagctgaagcttcgaggggctcgca |
| | tctctccttcacgcgcccgccgccctacctgaggccgccatccacgccggttgagtcgcgttctgccgcc |
| | tcccgcctgtggtgcctcctgaactgcgtccgccgtctaggtaaGTcgactcgttggatccCCACTACCC |
| | GGATCAACGCCCTAGGTTTATGTTTGGATGAACTGACATACGCGTATCCGTCTTAAGAATCTTTTCAAAC |
| | ACTAGTAGTGAAATATATATTAAACTAGTGTTTGAAAAGATTCTTATTACGGTAACGCGGAATTCGCAAC |
| | TATTTTATCAATTTTTTGCGTCGACACTTCAAGGGGCTTGCGGCCGCAACCATCTCCATGGCTGTTTGAA |
| | TGAGGCTTCAGTACTTTACAGAATCGTTGCCTGCACATCTTGGAAACACTTGCTGGGATTACTTCGACTT |
| | CTTAACCCAACAGAAGGCTCGAGAAGGTATATTGCTGTTGACAGTGAGCGCCAGTGTGAAGCTCTTGTCA |
| | GATAGTGAAGCCACAGATGTATCTGACAAGAGCTTCACACTGATGCCTACTGCCTCGGACTTCAAGGGGC |
| | TAGAATTCGAGCAATTATCTTGTTTACTAAAACTGAATACCTTGCTATCTCTTTGATACATTTTTACAAA |
| | GCTGAATTAAAATGGTATAAATTAAATCACTTTGACGGTGACTTAGGAGTATGCCGGATCAACGCCCTAG |
| | GTTTATGTTTGGATGAACTGACATACGCGTATCCGTCTAAAAATCAATCTCATTTCCTGGTAGTGAAATA |
| | TATATTAAACCAGGAAATGAGATTGATTTTTTTACGGTAACGCGGAATTCGCAACTATTTTATCAATTTT |
| | TTGCGTCGACGACTGTGACAGCAGAGTATGCCGGATCAACGCCCTAGGTTTATGTTTGGATGAACTGACA |
| | TACGCGTATCCGTCTTATGTAAAAGACAAACAATGCGTAGTGAAATATATATTAAACGCATTGTTTGTCT |
| | TTTACATATTACGGTAACGCGGAATTCGCAACTATTTTATCAATTTTTTGCGTCGACCGGAACTATCTTG |
| | AAGAGTAGTAGTGGactagtgtgacgctgctgacccctttctttcccttctACAGATCCAAGCTGTGACC |
| | GGCGCCTACacctgcagcccaagcttaccatggccttaccagtgaccgccttgctcctgccgctggcctt |
| | gctgctccacgccgccaggcctGATATTCAGATGACACAGAGCCCAAGTTCACTGTCAGCGAGTGTCGGA |
| | GATCGGGTTACTATAACCTGCCGAGCATCTCAGGATATCAATAATTACCTGGCCTGGTATCAGCAAAAAC |
| | CCGGCAAAGTCCCGAAGCTCCTGATATACGCAGCTTCAACACTGCAAAGCGGTGTGCCCTCGCGCTTTAG |
| | CGGCTCTGGCAGCGGGACAGATTTCACCCTGACGATTTCCTCCCTTCAACCCGAGGACGTGGCCACTTAC |
| | TACTCCCTCAACTATTACTCTGTACCCTGGACCTTTGGCCAAGGGACAAAGGTGGAAATCAAGGGCTCTA |
| | CTAGCGGTTCAGGGAAACCTGGGAGTGGAGAGGGCTCGACCAAGGGACAGGTCCAGCTCGTGGAGTCTGG |
| | CGGTGGGCTGGTTCAGCCAGGGAGGAGTTTGCGGCTTTCCTGTACGGGAAGCGGATTCACCTTTGGTGAC |
| | TATGCCATGAACTGGGTCAGGCAGGCTCCTGGAAAAGGCCTAGAGTGGGTGGGTTTCATCAGAACCAAGC |
| | CATATGGCGGCACTACAGAATATGCAGCCAGCGTAAAAGGGAGATTCACGTTCAGCCGCGACGACTCTAA |
| | GTCAATAGCTTACCTTCAGATGAACTCCCTCAAGACCGAAGACACCGCCGTGTACTATTGTACTATGATC |
| | CCTGTGCTGCGTTTTTTAGAGTGGTTGCCTTGGGGACAAGGGACATTAGTTACTGTGTCCTCCGCAACCA |
| | cgacgccagcgccgcgaccaccaacaccggcgcccaccatcgcgtcgcagcccctgtccctgcgcccTga |
| | ggcgtgcttcatgtacgtggcggcggccgcctttgtgcttctgttcttcgtgggctgcggggtgctgctg |
| | tcccgtaaacgCagacgtcaacacggtcaactgtggtttccagaaggttttaaggtctccgaagcaagta |
| | agaagaaaagacgtgaaccactgggagaagatagcgtcggtctgaaaccactcaagaatgccatggtttc |
| | taaactgagccagctgcagacggagctcctggcggccctgctggagtcagggctgagcaaagaggcactg |
| | ctccaggcactgggCgagccggggccctacctcctggctggagaaggccccctggacaagggggagtcct |
| | gcggcggcggtcgaggggagctggctgagctgcccaatgggctgggggagactcggggctccgaggacga |
| | gacCgacgacgatggggaagacttcacgccacccatcctcaaagagctggagaacctcagccctgaggag |
| | gcggcccaccagaaagccgtggtggagacccttctgcaggaggacccgtggcgtgtggcgaagatggtca |
| | agtcctacctgcagcagcacaacatcccacagcgggaggtggtcgataccactggcctcaaccagtccca |
| | cctgtcccaacacctcaacaagggcactcccatgaagacgcagaagcgggccgccctgtacacctggtaT |
| | gtccgcaagcagcgagaggtggcgcagcagttcacccatgcagggcagggagggctgattgaAgaGccca |
| | caggAgatgagctaccaaccaagaaggggcggaggaaccgtttcaagtggggcccagcatcccagcagat |
| | cctgttccaggcctatgagaggcagaagaaccctagcaaggaggagcgagaAacgctagtggaggagtgc |
| | aatagggcggaatgcatccagagaggTgtgtcAccatcacaAgcacaAggTctgggctccaacctcgtca |
| | cggaggtgcgtgtctacaactggtttgccaaccggcgcaaagaagaagccttccggcacaagctggccat |
| | gacctgcagggatgagtttcccaccatggtgtttccttctgggcagatcagccaggcctcggccttggcc |
| | ccggcccctccccaagtcctgccccaggctccagcccctgcccctgctccagccatggtatcagctctgg |
| | cccaggccccagcccctgtcccagtcctagccccaggccctcctcaAgctgtggccccacctgcccccaa |
| | gcccacccaAgctggggaaggaacgctgtcagaggccctgctgcagctgcagtttgatgatgaagacctg |
| | ggggccttgcttggcaacagcacagacccagctgtgttcacagacctggcatccgtcgacaactccgagt |
| | ttcagcagctgctgaaccagggcatacctgtggccccccacacaactgagcccatgctgatggagtaccc |
| | tgaggctataactcgcctagtgacaggggcccagaggccccccgacccagctcctgctccactgggggcc |
| | ccggggctccccaatggcctcctttcaggagatgaagacttctcctccattgcggacatggacttctcag |
| | ccctgctgagtcagatcagctcc |
| LG 239 | cgactgtgccttctagttgccagccatctgttgtttgcccctcccccgtgccttccttgaccctggaagg |
| core | tgccactcccactgtcctttcctaataaaatgaggaaattgcatcgcattgtctgagtaggtgtcattct |
| insert | attctggggggtggggtggggcaggacagcaagggggaggattgggaagacaatagcaggcatgctgggg |
| (w/o | atgcggtgggctctatgggataagcttgatatcgaattcatcgatgttaataattaacatatatgttaat |
| homology | cattaacatatagttaattattaaccgctatgttaatgattaacaacggttaataattaacatatatgtt |
| wings) | aatcattaacatataactagtctagagggtatataatgggggccactagtctactaccagagTtcatcgc |
| SEQ ID | tagcgctaccggatccgccaccATGGCCCTGCCAGTAACGGCTCTGCTGCTGCCACTTGCTCTGCTCCTC |
| NO: 1239 | CATGCAGCCAGGCCTCAGGTCCAGTTGGTACAGAGCGGCGCCGAAGTGAAAAAGCCTGGGGCGTCCGTCA |
| | AAGTGTCTTGCAAGGCCTCCGGCTATACATTCACCGGGTACTACATGCATTGGGTGCGGCAGGCACCTGG |
| | CCAGGGTCTAGAATGGATGGGCCGGATCAATCCCAACTCCGGCGGCACAAACTATGCTCAGAAATTTCAA |
| | GGTCGCGTCACCATGACCCGTGACACAAGTACGAGCACCGTCTACATGGAGCTGTCCTCCCTCAGGAGCG |
| | AGGATACAGCCGTGTACTATTGTGCAAGGGAGCGCGCCGGCTATAGCAGCGGGCAGTTCGATTATTGGGG |
| | ACAAGGGACTCTGGTAACTGTGTCCTCCGGAGGCGGAGGATCAGGCGGAGGAGGCTCAGGAGGTGGAGGT |
| | TCTGACATTCAGATGACTCAATCTCCCTCGTCACTGTCAGCTAGTGTTGGGGATAGAGTGACTATTACCT |
| | GCCGAGCCAGTCAGTCAATATCTAACTGGCTCGCATGGTACCAGCAGAAGCCAGGGAAGGCTCCCAAACT |
| | GCTGATCTACGCCGCGAGCACCCTTCAGAATGGCGTGCCGTCTAGATTTAGCGGTTCTGGGTCTGGGACC |
| | GACTTTACACTTACTATCAGTAGTTTACAACCAGAGGACTTTGCTACTTATTACTGTCAACAGAGCTACA |
| | CCTTCCCTATTACGTTCGGCCAGGGAACAAAAGTTGAAATCAAGGCGGCAGCAaccacgacgccagcgcc |
| | gcgaccaccaacaccggcgcccaccatcgcgtcgcagccactgtcactgcgcccagaagcgtgccggcca |
| | gcggcggggggcgcagtgcaTacgagggggctggacttcgcctgtgatatctacatctgggcgcccttgg |
| | ccgggacttgtggggtccttctcctgtcactggttatcaccctttactgcaaacggggcagaaagaaact |
| | cctgtatatattcaaacaaccatttatgagaccagtacaaactactcaagaagaggacggctgtagctgc |
| | cgatttccagaagaagaagaaggaggatgtgaactgagagtgaagttcagcaggagcgcagacgcccccg |
| | cgtaccagcagggccagaaccagctctataacgagctcaatctaggacgaagagaggagtacgatgtttt |
| | ggacaagaggcgtggccgggaccctgagatggggggaaagccgagaaggaagaaccctcaggaaggcctg |
| | tacaatgaactgcagaaagataagatggcggaggcctacagtgagattgggatgaaaggcgagcgccgga |
| | ggggcaaggggcacgatggcctttaccagggtctcagtacagccaccaaggacacctacgacgcccttca |
| | catgcaggccctgccccctaggtaaaatcaacctctggattacaaaatttgtgaaagattgactggtatt |
| | cttaactatgttgctccttttacgctatgtggatacgctgctttaatgcctttgtatcatgctattgctt |
| | cccgtatggctttcattttctcctccttgtataaatcctggttgctgtctctttatgaggagttgtggcc |
| | cgttgtcaggcaacgtggcgtggtCtgcactgtgtttgctgacgcaacccccactggttggggcattgcc |
| | accacctgtcagctcctttccgggactttcgctttccccctccctattgccacggcggaactcatcgccg |
| | cctgccttgcccgctgctggacaggggctcggctgttgggcactgacaattccgtggtgttgtcggggaa |
| | atcatcgtcctttccttggctgctcgcctgtgttgccacctggattctgcgcgggacgtccttctgctac |
| | gtcccttcggccctcaatccagcggaccttccttcccgcggcctgctgccggctctgcggcctcttccgc |
| | gtcttcgccttcgccctcagacgagtcggatctccctttgggccgcctccccgcctggatccttgacttg |
| | cggccaacttgtttattgcagcttataatggttacaaataaagcaatagcatcacaaatttcacaaataa |
| | agcatttttttcactgcattctagttgtggtttgtccaaactcatcaatgtatcttatcatgtctgggat |
| | ccttgacttgcggccgcaactcccacctgcaacatgcgtgactgactgaggccgcgactctagagtcgac |
| | cggatctgcgatcgctccggtgcccgtcagtgggcagagcgcacatcgcccacagtccccgagaagttgg |
| | ggggaggggtcggcaattgaacgggtgcctagagaaggtggcgcggggtaaactgggaaagtgatgtcgt |
| | gtactggctccgcctttttcccgagggtgggggagaaccgtatataagtgcagtagtcgccgtgaacgtt |
| | ctttttcgcaacgggtttgccgccagaacacagctgaagcttcgaggggctcgcatctctccttcacgcg |
| | cccgccgccctacctgaggccgccatccacgccggttgagtcgcgttctgccgcctcccgcctgtggtgc |
| | ctcctgaactgcgtccgccgtctaggtaaGTcgactcgttggatccCCACTACCCGGATCAACGCCCTAG |
| | GTTTATGTTTGGATGAACTGACATACGCGTATCCGTCTTAAGAATCTTTTCAAACACTAGTAGTGAAATA |
| | TATATTAAACTAGTGTTTGAAAAGATTCTTATTACGGTAACGCGGAATTCGCAACTATTTTATCAATTTT |
| | TTGCGTCGACACTTCAAGGGGCTTGCGGCCGCAACCATCTCCATGGCTGTTTGAATGAGGCTTCAGTACT |
| | TTACAGAATCGTTGCCTGCACATCTTGGAAACACTTGCTGGGATTACTTCGACTTCTTAACCCAACAGAA |
| | GGCTCGAGAAGGTATATTGCTGTTGACAGTGAGCGCCAGTGTGAAGCTCTTGTCAGATAGTGAAGCCACA |
| | GATGTATCTGACAAGAGCTTCACACTGATGCCTACTGCCTCGGACTTCAAGGGGCTAGAATTCGAGCAAT |
| | TATCTTGTTTACTAAAACTGAATACCTTGCTATCTCTTTGATACATTTTTACAAAGCTGAATTAAAATGG |
| | TATAAATTAAATCACTTTGACGGTGACTTAGGAGTATGCCGGATCAACGCCCTAGGTTTATGTTTGGATG |
| | AACTGACATACGCGTATCCGTCTAAAAATCAATCTCATTTCCTGGTAGTGAAATATATATTAAACCAGGA |
| | AATGAGATTGATTTTTTTACGGTAACGCGGAATTCGCAACTATTTTATCAATTTTTTGCGTCGACGACTG |
| | TGACAGCAGAGTATGCCGGATCAACGCCCTAGGTTTATGTTTGGATGAACTGACATACGCGTATCCGTCT |
| | TATGTAAAAGACAAACAATGCGTAGTGAAATATATATTAAACGCATTGTTTGTCTTTTACATATTACGGT |
| | AACGCGGAATTCGCAACTATTTTATCAATTTTTTGCGTCGACCGGAACTATCTTGAAGAGTAGTAGTGGa |
| | ctagtgtgacgctgctgacccctttctttcccttctACAGATCCAAGCTGTGACCGGCGCCTACacctgc |
| | agcccaagcttaccatggccttaccagtgaccgccttgctcctgccgctggccttgctgctccacgccgc |
| | caggcctGAGATAGTCCTGACCCAGTCACCCGATTTCCAGAGTGTTACTCCTAAGGAGAAAGTGACTATA |
| | ACATGCCGGGCATCTCAGTCTGTCGGAAGCGGGCTACATTGGTACCAGCAGAAGCCTGACCAGAGCCCGA |
| | AACTGCTCATCAAATATGCCTCCCAGTCGTTTTCTGGCGTGCCCTCTCGCTTTTCCGGAAGCGGATCTGG |
| | CACAGACTTCACCTTGACCATCAATAGCCTGGAAACTGAGGACGCCGCTACGTATTTCTGCCAGCAGTCC |
| | TCCAGTCTGCCTTGGACATTTGGTCAGGGAACGAAGGTGGAGATCAAGGGTTCAACATCAGGGAGCGGGA |
| | AACCGGGCTCTGGCGAGGGCTCAACAAAGGGACAAGTGCAACTGCAAGAATCGGGACCCGGGCTGGTTAA |
| | ACCAAGTGAAACCCTTTCCCTCACTTGTACCGTAAGCGGCGGTAGCGTGTCCTCTGGTAGCTACTATTGG |
| | AGTTGGATTAGGCAGGCGCCAGGGAAAGGCCTCGAATGGATTGGGTATATCTACTACAGCGGCAGTAACT |
| | ACTACAACCCATCATTGAAGTCTAGAGTGACAATTAGTGTCGATACCTCTAAAAATCAATTCTCACTTAA |
| | GCTGCGAGCTGTAACCGCCGCAGACACTGCGGTGTACTATTGTGCCCGTTGGATGACCACTATCAAGGGC |
| | TACTTCGATTATTGGGGACAAGGGACATTAGTTACGGTGTCCTCCGCAACCAcgacgccagcgccgcgac |
| | caccaacaccggcgcccaccatcgcgtcgcagcccctgtccctgcgcccTgaggcgtgcttcatgtacgt |
| | ggcggcggccgcctttgtgcttctgttcttcgtgggctgcggggtgctgctgtcccgtaaacgCagacgt |
| | caacacggtcaactgtggtttccagaaggttttaaggtctccgaagcaagtaagaagaaaagacgtgaac |
| | cactgggagaagatagcgtcggtctgaaaccactcaagaatgccatggtttctaaactgagccagctgca |
| | gacggagctcctggcggccctgctggagtcagggctgagcaaagaggcactgctccaggcactgggCgag |
| | ccggggccctacctcctggctggagaaggccccctggacaagggggagtcctgcggcggcggtcgagggg |
| | agctggctgagctgcccaatgggctgggggagactcggggctccgaggacgagacCgacgacgatgggga |
| | agacttcacgccacccatcctcaaagagctggagaacctcagccctgaggaggcggcccaccagaaagcc |
| | gtggtggagacccttctgcaggaggacccgtggcgtgtggcgaagatggtcaagtcctacctgcagcagc |
| | acaacatcccacagcgggaggtggtcgataccactggcctcaaccagtcccacctgtcccaacacctcaa |
| | caagggcactcccatgaagacgcagaagcgggccgccctgtacacctggtaTgtccgcaagcagcgagag |
| | gtggcgcagcagttcacccatgcagggcagggagggctgattgaAgaGcccacaggAgatgagctaccaa |
| | ccaagaaggggcggaggaaccgtttcaagtggggcccagcatcccagcagatcctgttccaggcctatga |
| | gaggcagaagaaccctagcaaggaggagcgagaAacgctagtggaggagtgcaatagggcggaatgcatc |
| | cagagaggTgtgtcAccatcacaAgcacaAggTctgggctccaacctcgtcacggaggtgcgtgtctaca |
| | actggtttgccaaccggcgcaaagaagaagccttccggcacaagctggccatgacctgcagggatgagtt |
| | tcccaccatggtgtttccttctgggcagatcagccaggcctcggccttggccccggcccctccccaagtc |
| | ctgccccaggctccagcccctgcccctgctccagccatggtatcagctctggcccaggccccagcccctg |
| | tcccagtcctagccccaggccctcctcaAgctgtggccccacctgcccccaagcccacccaAgctgggga |
| | aggaacgctgtcagaggccctgctgcagctgcagtttgatgatgaagacctgggggccttgcttggcaac |
| | agcacagacccagctgtgttcacagacctggcatccgtcgacaactccgagtttcagcagctgctgaacc |
| | agggcatacctgtggccccccacacaactgagcccatgctgatggagtaccctgaggctataactcgcct |
| | agtgacaggggcccagaggccccccgacccagctcctgctccactgggggccccggggctccccaatggc |
| | ctcctttcaggagatgaagacttctcctccattgcggacatggacttctcagccctgctgagtcagatca |
| | gctcc |
| LG 39 | cgactgtgccttctagttgccagccatctgttgtttgcccctcccccgtgccttccttgaccctggaagg |
| core | tgccactcccactgtcctttcctaataaaatgaggaaattgcatcgcattgtctgagtaggtgtcattct |
| insert | attctggggggtggggtggggcaggacagcaagggggaggattgggaagacaatagcaggcatgctgggg |
| (w/o | atgcggtgggctctatgggataagcttgatatcgaattcatcgatgttaataattaacatatatgttaat |
| homology | cattaacatatagttaattattaaccgctatgttaatgattaacaacggttaataattaacatatatgtt |
| wings) | aatcattaacatataactagtctagagggtatataatgggggccactagtctactaccagagTtcatcgc |
| SEQ ID | tagcgctaccggatccgccaccATGGCCCTGCCAGTAACGGCTCTGCTGCTGCCACTTGCTCTGCTCCTC |
| NO: 1240 | CATGCAGCCAGGCCTCAGGTCCAGTTGGTACAGAGCGGCGCCGAAGTGAAAAAGCCTGGGGCGTCCGTCA |
| | AAGTGTCTTGCAAGGCCTCCGGCTATACATTCACCGGGTACTACATGCATTGGGTGCGGCAGGCACCTGG |
| | CCAGGGTCTAGAATGGATGGGCCGGATCAATCCCAACTCCGGCGGCACAAACTATGCTCAGAAATTTCAA |
| | GGTCGCGTCACCATGACCCGTGACACAAGTACGAGCACCGTCTACATGGAGCTGTCCTCCCTCAGGAGCG |
| | AGGATACAGCCGTGTACTATTGTGCAAGGGAGCGCGCCGGCTATAGCAGCGGGCAGTTCGATTATTGGGG |
| | ACAAGGGACTCTGGTAACTGTGTCCTCCGGAGGCGGAGGATCAGGCGGAGGAGGCTCAGGAGGTGGAGGT |
| | TCTGACATTCAGATGACTCAATCTCCCTCGTCACTGTCAGCTAGTGTTGGGGATAGAGTGACTATTACCT |
| | GCCGAGCCAGTCAGTCAATATCTAACTGGCTCGCATGGTACCAGCAGAAGCCAGGGAAGGCTCCCAAACT |
| | GCTGATCTACGCCGCGAGCACCCTTCAGAATGGCGTGCCGTCTAGATTTAGCGGTTCTGGGTCTGGGACC |
| | GACTTTACACTTACTATCAGTAGTTTACAACCAGAGGACTTTGCTACTTATTACTGTCAACAGAGCTACA |
| | CCTTCCCTATTACGTTCGGCCAGGGAACAAAAGTTGAAATCAAGGCGGCAGCAaccacgacgccagcgcc |
| | gcgaccaccaacaccggcgcccaccatcgcgtcgcagccactgtcactgcgcccagaagcgtgccggcca |
| | gcggcggggggcgcagtgcaTacgagggggctggacttcgcctgtgatatctacatctgggcgcccttgg |
| | ccgggacttgtggggtccttctcctgtcactggttatcaccctttactgcaaacggggcagaaagaaact |
| | cctgtatatattcaaacaaccatttatgagaccagtacaaactactcaagaagaggacggctgtagctgc |
| | cgatttccagaagaagaagaaggaggatgtgaactgagagtgaagttcagcaggagcgcagacgcccccg |
| | cgtaccagcagggccagaaccagctctataacgagctcaatctaggacgaagagaggagtacgatgtttt |
| | ggacaagaggcgtggccgggaccctgagatggggggaaagccgagaaggaagaaccctcaggaaggcctg |
| | tacaatgaactgcagaaagataagatggcggaggcctacagtgagattgggatgaaaggcgagcgccgga |
| | ggggcaaggggcacgatggcctttaccagggtctcagtacagccaccaaggacacctacgacgcccttca |
| | catgcaggccctgccccctaggtaaaatcaacctctggattacaaaatttgtgaaagattgactggtatt |
| | cttaactatgttgctccttttacgctatgtggatacgctgctttaatgcctttgtatcatgctattgctt |
| | cccgtatggctttcattttctcctccttgtataaatcctggttgctgtctctttatgaggagttgtggcc |
| | cgttgtcaggcaacgtggcgtggtCtgcactgtgtttgctgacgcaacccccactggttggggcattgcc |
| | accacctgtcagctcctttccgggactttcgctttccccctccctattgccacggcggaactcatcgccg |
| | cctgccttgcccgctgctggacaggggctcggctgttgggcactgacaattccgtggtgttgtcggggaa |
| | atcatcgtcctttccttggctgctcgcctgtgttgccacctggattctgcgcgggacgtccttctgctac |
| | gtcccttcggccctcaatccagcggaccttccttcccgcggcctgctgccggctctgcggcctcttccgc |
| | gtcttcgccttcgccctcagacgagtcggatctccctttgggccgcctccccgcctggatccttgacttg |
| | cggccaacttgtttattgcagcttataatggttacaaataaagcaatagcatcacaaatttcacaaataa |
| | agcatttttttcactgcattctagttgtggtttgtccaaactcatcaatgtatcttatcatgtctgggat |
| | ccttgacttgcggccgcaactcccacctgcaacatgcgtgactgactgaggccgcgactctagagtcgac |
| | cggatctgcgatcgctccggtgcccgtcagtgggcagagcgcacatcgcccacagtccccgagaagttgg |
| | ggggaggggtcggcaattgaacgggtgcctagagaaggtggcgcggggtaaactgggaaagtgatgtcgt |
| | gtactggctccgcctttttcccgagggtgggggagaaccgtatataagtgcagtagtcgccgtgaacgtt |
| | ctttttcgcaacgggtttgccgccagaacacagctgaagcttcgaggggctcgcatctctccttcacgcg |
| | cccgccgccctacctgaggccgccatccacgccggttgagtcgcgttctgccgcctcccgcctgtggtgc |
| | ctcctgaactgcgtccgccgtctaggtaaGTcgactcgttggatccCCACTACCCGGATCAACGCCCTAG |
| | GTTTATGTTTGGATGAACTGACATACGCGTATCCGTCTTAAGAATCTTTTCAAACACTAGTAGTGAAATA |
| | TATATTAAACTAGTGTTTGAAAAGATTCTTATTACGGTAACGCGGAATTCGCAACTATTTTATCAATTTT |
| | TTGCGTCGACACTTCAAGGGGCTTGCGGCCGCAACCATCTCCATGGCTGTTTGAATGAGGCTTCAGTACT |
| | TTACAGAATCGTTGCCTGCACATCTTGGAAACACTTGCTGGGATTACTTCGACTTCTTAACCCAACAGAA |
| | GGCTCGAGAAGGTATATTGCTGTTGACAGTGAGCGCCAGTGTGAAGCTCTTGTCAGATAGTGAAGCCACA |
| | GATGTATCTGACAAGAGCTTCACACTGATGCCTACTGCCTCGGACTTCAAGGGGCTAGAATTCGAGCAAT |
| | TATCTTGTTTACTAAAACTGAATACCTTGCTATCTCTTTGATACATTTTTACAAAGCTGAATTAAAATGG |
| | TATAAATTAAATCACTTTGACGGTGACTTAGGAGTATGCCGGATCAACGCCCTAGGTTTATGTTTGGATG |
| | AACTGACATACGCGTATCCGTCTAAAAATCAATCTCATTTCCTGGTAGTGAAATATATATTAAACCAGGA |
| | AATGAGATTGATTTTTTTACGGTAACGCGGAATTCGCAACTATTTTATCAATTTTTTGCGTCGACGACTG |
| | TGACAGCAGAGTATGCCGGATCAACGCCCTAGGTTTATGTTTGGATGAACTGACATACGCGTATCCGTCT |
| | TATGTAAAAGACAAACAATGCGTAGTGAAATATATATTAAACGCATTGTTTGTCTTTTACATATTACGGT |
| | AACGCGGAATTCGCAACTATTTTATCAATTTTTTGCGTCGACCGGAACTATCTTGAAGAGTAGTAGTGGa |
| | ctagtgtgacgctgctgacccctttctttcccttctACAGATCCAAGCTGTGACCGGCGCCTACacctgc |
| | agcccaagcttaccatggccttaccagtgaccgccttgctcctgccgctggccttgctgctccacgccgc |
| | caggcctGAAATCGTCCTGACACAGTCTCCAGATTTTCAGAGCGTGACGCCAAAGGAGAAAGTGACAATT |
| | ACATGCCGGGCATCTCAGTCTGTTGGGTCTGGGTTGCATTGGTATCAGCAAAAGCCCGACCAGTCACCCA |
| | AACTGCTCATCAAATATGCAAGCCAGAGTTTTTCAGGCGTACCTTCACGATTTAGCGGAAGTGGTTCTGG |
| | CACTGACTTCACCTTGACGATTAATAGCCTGGAAGTAGAAGACGCTGCCACTTTCTACTGCCTGCAAAGT |
| | AGCTCCCTGCCCTGGACTTTTGGGCAGGGTACTAAGGTCGAGATCAAGGGCTCGACAAGCGGAAGTGGCA |
| | AACCGGGCAGCGGCGAGGGAAGCACCAAGGGACAAGTGCAACTGCAAGAGTCTGGACCCGGGCTGGTGAA |
| | ACCAAGCGAGACATTATCCCTCACTTGTACCGTGTCAGGCGGTAGTGTGTCCTCCGGGAATTTCTACTGG |
| | AGTTGGATACGCCAGCCTCCTGGGAAGGGCCTTGAATGGATTGGCTACATCTACTATTCAGGCTCCACCT |
| | ACTACAACCCGTCTTTGAAGTCAAGGGTTACGATAAGCGTCGATACCTCCAAAAACCAATTCTCCCTAAA |
| | GCTCAGATCGTTAACTGCCGCTGATACCGCGGTGTACTATTGTGCCCGTTGGATGACCAAAGTTAAGGGT |
| | TATTTCGACTATTGGGGACAAGGGACACTTGTCACCGTGTCCTCCGCAACCAcgacgccagcgccgcgac |
| | caccaacaccggcgcccaccatcgcgtcgcagcccctgtccctgcgcccTgaggcgtgcttcatgtacgt |
| | ggcggcggccgcctttgtgcttctgttcttcgtgggctgcggggtgctgctgtcccgtaaacgCagacgt |
| | caacacggtcaactgtggtttccagaaggttttaaggtctccgaagcaagtaagaagaaaagacgtgaac |
| | cactgggagaagatagcgtcggtctgaaaccactcaagaatgccatggtttctaaactgagccagctgca |
| | gacggagctcctggcggccctgctggagtcagggctgagcaaagaggcactgctccaggcactgggCgag |
| | ccggggccctacctcctggctggagaaggccccctggacaagggggagtcctgcggcggcggtcgagggg |
| | agctggctgagctgcccaatgggctgggggagactcggggctccgaggacgagacCgacgacgatgggga |
| | agacttcacgccacccatcctcaaagagctggagaacctcagccctgaggaggcggcccaccagaaagcc |
| | gtggtggagacccttctgcaggaggacccgtggcgtgtggcgaagatggtcaagtcctacctgcagcagc |
| | acaacatcccacagcgggaggtggtcgataccactggcctcaaccagtcccacctgtcccaacacctcaa |
| | caagggcactcccatgaagacgcagaagcgggccgccctgtacacctggtaTgtccgcaagcagcgagag |
| | gtggcgcagcagttcacccatgcagggcagggagggctgattgaAgaGcccacaggAgatgagctaccaa |
| | ccaagaaggggcggaggaaccgtttcaagtggggcccagcatcccagcagatcctgttccaggcctatga |
| | gaggcagaagaaccctagcaaggaggagcgagaAacgctagtggaggagtgcaatagggcggaatgcatc |
| | cagagaggTgtgtcAccatcacaAgcacaAggTctgggctccaacctcgtcacggaggtgcgtgtctaca |
| | actggtttgccaaccggcgcaaagaagaagccttccggcacaagctggccatgacctgcagggatgagtt |
| | tcccaccatggtgtttccttctgggcagatcagccaggcctcggccttggccccggcccctccccaagtc |
| | ctgccccaggctccagcccctgcccctgctccagccatggtatcagctctggcccaggccccagcccctg |
| | tcccagtcctagccccaggccctcctcaAgctgtggccccacctgcccccaagcccacccaAgctgggga |
| | aggaacgctgtcagaggccctgctgcagctgcagtttgatgatgaagacctgggggccttgcttggcaac |
| | agcacagacccagctgtgttcacagacctggcatccgtcgacaactccgagtttcagcagctgctgaacc |
| | agggcatacctgtggccccccacacaactgagcccatgctgatggagtaccctgaggctataactcgcct |
| | agtgacaggggcccagaggccccccgacccagctcctgctccactgggggccccggggctccccaatggc |
| | ctcctttcaggagatgaagacttctcctccattgcggacatggacttctcagccctgctgagtcagatca |
| | gctcc |
| LG 43 | cgactgtgccttctagttgccagccatctgttgtttgcccctcccccgtgccttccttgaccctggaagg |
| core | tgccactcccactgtcctttcctaataaaatgaggaaattgcatcgcattgtctgagtaggtgtcattct |
| insert | attctggggggtggggtggggcaggacagcaagggggaggattgggaagacaatagcaggcatgctgggg |
| (w/o | atgcggtgggctctatgggataagcttgatatcgaattcatcgatgttaataattaacatatatgttaat |
| homology | cattaacatatagttaattattaaccgctatgttaatgattaacaacggttaataattaacatatatgtt |
| wings) | aatcattaacatataactagtctagagggtatataatgggggccactagtctactaccagagTtcatcgc |
| SEQ ID | tagcgctaccggatccgccaccATGGCCCTGCCAGTAACGGCTCTGCTGCTGCCACTTGCTCTGCTCCTC |
| NO: 1241 | CATGCAGCCAGGCCTCAAGTGCAGCTGGTCCAGTCTGGGGCGGAAGTGAAAAAGCCCGGAGCTAGTGTAA |
| | AGGTGTCCTGTAAAGCCAGCGGCTACACCTTCACCGGTTATTACCTGCATTGGGTCCGGCAGGCTCCTGG |
| | CCAGGGCCTGGAGTGGATGGGCTGGATTTCCGCATATAACGGAAACACAAATTACGCCCAGAACCTGCAA |
| | GGCCGCGTGACCATGACCAGGGACACAAGCACTAGCACTGTCTACATGGAGTTGTCTAGCTTGAGAAGCG |
| | AAGATACCGCTGTGTACTATTGCGCCCGACACTCTTACTCGGGCTCATACTCAACGCTACCCTATTATGG |
| | GATGGATGTTTGGGGTCAAGGGACAACGGTCACAGTATCCTCTGGAGGCGGTGGCAGCGGAGGAGGCGGG |
| | TCTGGAGGTGGTGGATCAGACATTCAGATGACCCAGTCACCAAGTTCCTTATCCGCAAGCGTTGGGGATC |
| | GTGTTACAATTACTTGCAGGGCCTCGCAAGGGATCTCTAATTATCTCGCTTGGTACCAGCAGAAACCTGG |
| | GAAAGCACCCAAGCTGCTGATCTACACTGCAAGCACACTTTTTCCAGGAGTGCCGTCAAGATTCTCTGGG |
| | TCCGGGAGTGGCACTGACTTCACCCTTACCATCTCCTCCCTCCAGCCTGAGGACTTTGCCACATATTATT |
| | GTCAACAGAGTTACTCCATACCACTCACGTTTGGCGGCGGAACAAAaGTtGAAATCAAGGCGGCAGCAac |
| | cacgacgccagcgccgcgaccaccaacaccggcgcccaccatcgcgtcgcagccactgtcactgcgccca |
| | gaagcgtgccggccagcggcggggggcgcagtgcaTacgagggggctggacttcgcctgtgatatctaca |
| | tctgggcgcccttggccgggacttgtggggtccttctcctgtcactggttatcaccctttactgcaaacg |
| | gggcagaaagaaactcctgtatatattcaaacaaccatttatgagaccagtacaaactactcaagaagag |
| | gacggctgtagctgccgatttccagaagaagaagaaggaggatgtgaactgagagtgaagttcagcagga |
| | gcgcagacgcccccgcgtaccagcagggccagaaccagctctataacgagctcaatctaggacgaagaga |
| | ggagtacgatgttttggacaagaggcgtggccgggaccctgagatggggggaaagccgagaaggaagaac |
| | cctcaggaaggcctgtacaatgaactgcagaaagataagatggcggaggcctacagtgagattgggatga |
| | aaggcgagcgccggaggggcaaggggcacgatggcctttaccagggtctcagtacagccaccaaggacac |
| | ctacgacgcccttcacatgcaggccctgccccctaggtaaaatcaacctctggattacaaaatttgtgaa |
| | agattgactggtattcttaactatgttgctccttttacgctatgtggatacgctgctttaatgcctttgt |
| | atcatgctattgcttcccgtatggctttcattttctcctccttgtataaatcctggttgctgtctcttta |
| | tgaggagttgtggcccgttgtcaggcaacgtggcgtggtCtgcactgtgtttgctgacgcaacccccact |
| | ggttggggcattgccaccacctgtcagctcctttccgggactttcgctttccccctccctattgccacgg |
| | cggaactcatcgccgcctgccttgcccgctgctggacaggggctcggctgttgggcactgacaattccgt |
| | ggtgttgtcggggaaatcatcgtcctttccttggctgctcgcctgtgttgccacctggattctgcgcggg |
| | acgtccttctgctacgtcccttcggccctcaatccagcggaccttccttcccgcggcctgctgccggctc |
| | tgcggcctcttccgcgtcttcgccttcgccctcagacgagtcggatctccctttgggccgcctccccgcc |
| | tggatccttgacttgcggccaacttgtttattgcagcttataatggttacaaataaagcaatagcatcac |
| | aaatttcacaaataaagcatttttttcactgcattctagttgtggtttgtccaaactcatcaatgtatct |
| | tatcatgtctgggatccttgacttgcggccgcaactcccacctgcaacatgcgtgactgactgaggccgc |
| | gactctagagtcgaccggatctgcgatcgctccggtgcccgtcagtgggcagagcgcacatcgcccacag |
| | tccccgagaagttggggggaggggtcggcaattgaacgggtgcctagagaaggtggcgcggggtaaactg |
| | ggaaagtgatgtcgtgtactggctccgcctttttcccgagggtgggggagaaccgtatataagtgcagta |
| | gtcgccgtgaacgttctttttcgcaacgggtttgccgccagaacacagctgaagcttcgaggggctcgca |
| | tctctccttcacgcgcccgccgccctacctgaggccgccatccacgccggttgagtcgcgttctgccgcc |
| | tcccgcctgtggtgcctcctgaactgcgtccgccgtctaggtaaGTcgactcgttggatccCCACTACCC |
| | GGATCAACGCCCTAGGTTTATGTTTGGATGAACTGACATACGCGTATCCGTCTTAAGAATCTTTTCAAAC |
| | ACTAGTAGTGAAATATATATTAAACTAGTGTTTGAAAAGATTCTTATTACGGTAACGCGGAATTCGCAAC |
| | TATTTTATCAATTTTTTGCGTCGACACTTCAAGGGGCTTGCGGCCGCAACCATCTCCATGGCTGTTTGAA |
| | TGAGGCTTCAGTACTTTACAGAATCGTTGCCTGCACATCTTGGAAACACTTGCTGGGATTACTTCGACTT |
| | CTTAACCCAACAGAAGGCTCGAGAAGGTATATTGCTGTTGACAGTGAGCGCCAGTGTGAAGCTCTTGTCA |
| | GATAGTGAAGCCACAGATGTATCTGACAAGAGCTTCACACTGATGCCTACTGCCTCGGACTTCAAGGGGC |
| | TAGAATTCGAGCAATTATCTTGTTTACTAAAACTGAATACCTTGCTATCTCTTTGATACATTTTTACAAA |
| | GCTGAATTAAAATGGTATAAATTAAATCACTTTGACGGTGACTTAGGAGTATGCCGGATCAACGCCCTAG |
| | GTTTATGTTTGGATGAACTGACATACGCGTATCCGTCTAAAAATCAATCTCATTTCCTGGTAGTGAAATA |
| | TATATTAAACCAGGAAATGAGATTGATTTTTTTACGGTAACGCGGAATTCGCAACTATTTTATCAATTTT |
| | TTGCGTCGACGACTGTGACAGCAGAGTATGCCGGATCAACGCCCTAGGTTTATGTTTGGATGAACTGACA |
| | TACGCGTATCCGTCTTATGTAAAAGACAAACAATGCGTAGTGAAATATATATTAAACGCATTGTTTGTCT |
| | TTTACATATTACGGTAACGCGGAATTCGCAACTATTTTATCAATTTTTTGCGTCGACCGGAACTATCTTG |
| | AAGAGTAGTAGTGGactagtgtgacgctgctgacccctttctttcccttctACAGATCCAAGCTGTGACC |
| | GGCGCCTACacctgcagcccaagcttaccatggccttaccagtgaccgccttgctcctgccgctggcctt |
| | gctgctccacgccgccaggcctGAGATAGTCCTGACCCAGTCACCCGATTTCCAGAGTGTTACTCCTAAG |
| | GAGAAAGTGACTATAACATGCCGGGCATCTCAGTCTGTCGGAAGCGGGCTACATTGGTACCAGCAGAAGC |
| | CTGACCAGAGCCCGAAACTGCTCATCAAATATGCCTCCCAGTCGTTTTCTGGCGTGCCCTCTCGCTTTTC |
| | CGGAAGCGGATCTGGCACAGACTTCACCTTGACCATCAATAGCCTGGAAACTGAGGACGCCGCTACGTAT |
| | TTCTGCCAGCAGTCCTCCAGTCTGCCTTGGACATTTGGTCAGGGAACGAAGGTGGAGATCAAGGGTTCAA |
| | CATCAGGGAGCGGGAAACCGGGCTCTGGCGAGGGCTCAACAAAGGGACAAGTGCAACTGCAAGAATCGGG |
| | ACCCGGGCTGGTTAAACCAAGTGAAACCCTTTCCCTCACTTGTACCGTAAGCGGCGGTAGCGTGTCCTCT |
| | GGTAGCTACTATTGGAGTTGGATTAGGCAGGCGCCAGGGAAAGGCCTCGAATGGATTGGGTATATCTACT |
| | ACAGCGGCAGTAACTACTACAACCCATCATTGAAGTCTAGAGTGACAATTAGTGTCGATACCTCTAAAAA |
| | TCAATTCTCACTTAAGCTGCGAGCTGTAACCGCCGCAGACACTGCGGTGTACTATTGTGCCCGTTGGATG |
| | ACCACTATCAAGGGCTACTTCGATTATTGGGGACAAGGGACATTAGTTACGGTGTCCTCCGCAACCAcga |
| | cgccagcgccgcgaccaccaacaccggcgcccaccatcgcgtcgcagcccctgtccctgcgcccTgaggc |
| | gtgcttcatgtacgtggcggcggccgcctttgtgcttctgttcttcgtgggctgcggggtgctgctgtcc |
| | cgtaaacgCagacgtcaacacggtcaactgtggtttccagaaggttttaaggtctccgaagcaagtaaga |
| | agaaaagacgtgaaccactgggagaagatagcgtcggtctgaaaccactcaagaatgccatggtttctaa |
| | actgagccagctgcagacggagctcctggcggccctgctggagtcagggctgagcaaagaggcactgctc |
| | caggcactgggCgagccggggccctacctcctggctggagaaggccccctggacaagggggagtcctgcg |
| | gcggcggtcgaggggagctggctgagctgcccaatgggctgggggagactcggggctccgaggacgagac |
| | Cgacgacgatggggaagacttcacgccacccatcctcaaagagctggagaacctcagccctgaggaggcg |
| | gcccaccagaaagccgtggtggagacccttctgcaggaggacccgtggcgtgtggcgaagatggtcaagt |
| | cctacctgcagcagcacaacatcccacagcgggaggtggtcgataccactggcctcaaccagtcccacct |
| | gtcccaacacctcaacaagggcactcccatgaagacgcagaagcgggccgccctgtacacctggtaTgtc |
| | cgcaagcagcgagaggtggcgcagcagttcacccatgcagggcagggagggctgattgaAgaGcccacag |
| | gAgatgagctaccaaccaagaaggggcggaggaaccgtttcaagtggggcccagcatcccagcagatcct |
| | gttccaggcctatgagaggcagaagaaccctagcaaggaggagcgagaAacgctagtggaggagtgcaat |
| | agggcggaatgcatccagagaggTgtgtcAccatcacaAgcacaAggTctgggctccaacctcgtcacgg |
| | aggtgcgtgtctacaactggtttgccaaccggcgcaaagaagaagccttccggcacaagctggccatgac |
| | ctgcagggatgagtttcccaccatggtgtttccttctgggcagatcagccaggcctcggccttggccccg |
| | gcccctccccaagtcctgccccaggctccagcccctgcccctgctccagccatggtatcagctctggccc |
| | aggccccagcccctgtcccagtcctagccccaggccctcctcaAgctgtggccccacctgcccccaagcc |
| | cacccaAgctggggaaggaacgctgtcagaggccctgctgcagctgcagtttgatgatgaagacctgggg |
| | gccttgcttggcaacagcacagacccagctgtgttcacagacctggcatccgtcgacaactccgagtttc |
| | agcagctgctgaaccagggcatacctgtggccccccacacaactgagcccatgctgatggagtaccctga |
| | ggctataactcgcctagtgacaggggcccagaggccccccgacccagctcctgctccactgggggccccg |
| | gggctccccaatggcctcctttcaggagatgaagacttctcctccattgcggacatggacttctcagccc |
| | tgctgagtcagatcagctcc |
| LG 47 | cgactgtgccttctagttgccagccatctgttgtttgcccctcccccgtgccttccttgaccctggaagg |
| core | tgccactcccactgtcctttcctaataaaatgaggaaattgcatcgcattgtctgagtaggtgtcattct |
| insert | attctggggggtggggtggggcaggacagcaagggggaggattgggaagacaatagcaggcatgctgggg |
| (w/o | atgcggtgggctctatgggataagcttgatatcgaattcatcgatgttaataattaacatatatgttaat |
| homology | cattaacatatagttaattattaaccgctatgttaatgattaacaacggttaataattaacatatatgtt |
| wings) | aatcattaacatataactagtctagagggtatataatgggggccactagtctactaccagagTtcatcgc |
| SEQ ID | tagcgctaccggatccgccaccATGGCCCTGCCAGTAACGGCTCTGCTGCTGCCACTTGCTCTGCTCCTC |
| NO: 1242 | CATGCAGCCAGGCCTCAGGTCCAGTTGGTACAGAGCGGCGCCGAAGTGAAAAAGCCTGGGGCGTCCGTCA |
| | AAGTGTCTTGCAAGGCCTCCGGCTATACATTCACCGGGTACTACATGCATTGGGTGCGGCAGGCACCTGG |
| | CCAGGGTCTAGAATGGATGGGCCGGATCAATCCCAACTCCGGCGGCACAAACTATGCTCAGAAATTTCAA |
| | GGTCGCGTCACCATGACCCGTGACACAAGTACGAGCACCGTCTACATGGAGCTGTCCTCCCTCAGGAGCG |
| | AGGATACAGCCGTGTACTATTGTGCAAGGGAGCGCGCCGGCTATAGCAGCGGGCAGTTCGATTATTGGGG |
| | ACAAGGGACTCTGGTAACTGTGTCCTCCGGAGGCGGAGGATCAGGCGGAGGAGGCTCAGGAGGTGGAGGT |
| | TCTGACATTCAGATGACTCAATCTCCCTCGTCACTGTCAGCTAGTGTTGGGGATAGAGTGACTATTACCT |
| | GCCGAGCCAGTCAGTCAATATCTAACTGGCTCGCATGGTACCAGCAGAAGCCAGGGAAGGCTCCCAAACT |
| | GCTGATCTACGCCGCGAGCACCCTTCAGAATGGCGTGCCGTCTAGATTTAGCGGTTCTGGGTCTGGGACC |
| | GACTTTACACTTACTATCAGTAGTTTACAACCAGAGGACTTTGCTACTTATTACTGTCAACAGAGCTACA |
| | CCTTCCCTATTACGTTCGGCCAGGGAACAAAAGTTGAAATCAAGGCGGCAGCAaccacgacgccagcgcc |
| | gcgaccaccaacaccggcgcccaccatcgcgtcgcagccactgtcactgcgcccagaagcgtgccggcca |
| | gcggcggggggcgcagtgcaTacgagggggctggacttcgcctgtgatatctacatctgggcgcccttgg |
| | ccgggacttgtggggtccttctcctgtcactggttatcaccctttactgcaaacggggcagaaagaaact |
| | cctgtatatattcaaacaaccatttatgagaccagtacaaactactcaagaagaggacggctgtagctgc |
| | cgatttccagaagaagaagaaggaggatgtgaactgagagtgaagttcagcaggagcgcagacgcccccg |
| | cgtaccagcagggccagaaccagctctataacgagctcaatctaggacgaagagaggagtacgatgtttt |
| | ggacaagaggcgtggccgggaccctgagatggggggaaagccgagaaggaagaaccctcaggaaggcctg |
| | tacaatgaactgcagaaagataagatggcggaggcctacagtgagattgggatgaaaggcgagcgccgga |
| | ggggcaaggggcacgatggcctttaccagggtctcagtacagccaccaaggacacctacgacgcccttca |
| | catgcaggccctgccccctaggtaaaatcaacctctggattacaaaatttgtgaaagattgactggtatt |
| | cttaactatgttgctccttttacgctatgtggatacgctgctttaatgcctttgtatcatgctattgctt |
| | cccgtatggctttcattttctcctccttgtataaatcctggttgctgtctctttatgaggagttgtggcc |
| | cgttgtcaggcaacgtggcgtggtCtgcactgtgtttgctgacgcaacccccactggttggggcattgcc |
| | accacctgtcagctcctttccgggactttcgctttccccctccctattgccacggcggaactcatcgccg |
| | cctgccttgcccgctgctggacaggggctcggctgttgggcactgacaattccgtggtgttgtcggggaa |
| | atcatcgtcctttccttggctgctcgcctgtgttgccacctggattctgcgcgggacgtccttctgctac |
| | gtcccttcggccctcaatccagcggaccttccttcccgcggcctgctgccggctctgcggcctcttccgc |
| | gtcttcgccttcgccctcagacgagtcggatctccctttgggccgcctccccgcctggatccttgacttg |
| | cggccaacttgtttattgcagcttataatggttacaaataaagcaatagcatcacaaatttcacaaataa |
| | agcatttttttcactgcattctagttgtggtttgtccaaactcatcaatgtatcttatcatgtctgggat |
| | ccttgacttgcggccgcaactcccacctgcaacatgcgtgactgactgaggccgcgactctagagtcgac |
| | cggatctgcgatcgctccggtgcccgtcagtgggcagagcgcacatcgcccacagtccccgagaagttgg |
| | ggggaggggtcggcaattgaacgggtgcctagagaaggtggcgcggggtaaactgggaaagtgatgtcgt |
| | gtactggctccgcctttttcccgagggtgggggagaaccgtatataagtgcagtagtcgccgtgaacgtt |
| | ctttttcgcaacgggtttgccgccagaacacagctgaagcttcgaggggctcgcatctctccttcacgcg |
| | cccgccgccctacctgaggccgccatccacgccggttgagtcgcgttctgccgcctcccgcctgtggtgc |
| | ctcctgaactgcgtccgccgtctaggtaaGTcgactcgttggatccCCACTACCCGGATCAACGCCCTAG |
| | GTTTATGTTTGGATGAACTGACATACGCGTATCCGTCTTAAGAATCTTTTCAAACACTAGTAGTGAAATA |
| | TATATTAAACTAGTGTTTGAAAAGATTCTTATTACGGTAACGCGGAATTCGCAACTATTTTATCAATTTT |
| | TTGCGTCGACACTTCAAGGGGCTTGCGGCCGCAACCATCTCCATGGCTGTTTGAATGAGGCTTCAGTACT |
| | TTACAGAATCGTTGCCTGCACATCTTGGAAACACTTGCTGGGATTACTTCGACTTCTTAACCCAACAGAA |
| | GGCTCGAGAAGGTATATTGCTGTTGACAGTGAGCGCCAGTGTGAAGCTCTTGTCAGATAGTGAAGCCACA |
| | GATGTATCTGACAAGAGCTTCACACTGATGCCTACTGCCTCGGACTTCAAGGGGCTAGAATTCGAGCAAT |
| | TATCTTGTTTACTAAAACTGAATACCTTGCTATCTCTTTGATACATTTTTACAAAGCTGAATTAAAATGG |
| | TATAAATTAAATCACTTTGACGGTGACTTAGGAGTATGCCGGATCAACGCCCTAGGTTTATGTTTGGATG |
| | AACTGACATACGCGTATCCGTCTAAAAATCAATCTCATTTCCTGGTAGTGAAATATATATTAAACCAGGA |
| | AATGAGATTGATTTTTTTACGGTAACGCGGAATTCGCAACTATTTTATCAATTTTTTGCGTCGACGACTG |
| | TGACAGCAGAGTATGCCGGATCAACGCCCTAGGTTTATGTTTGGATGAACTGACATACGCGTATCCGTCT |
| | TATGTAAAAGACAAACAATGCGTAGTGAAATATATATTAAACGCATTGTTTGTCTTTTACATATTACGGT |
| | AACGCGGAATTCGCAACTATTTTATCAATTTTTTGCGTCGACCGGAACTATCTTGAAGAGTAGTAGTGGa |
| | ctagtgtgacgctgctgacccctttctttcccttctACAGATCCAAGCTGTGACCGGCGCCTACacctgc |
| | agcccaagcttaccatggccttaccagtgaccgccttgctcctgccgctggccttgctgctccacgccgc |
| | caggcctGAGATAGTCCTGACCCAGTCACCCGATTTCCAGAGTGTTACTCCTAAGGAGAAAGTGACTATA |
| | ACATGCCGGGCATCTCAGTCTGTCGGAAGCGGGCTACATTGGTACCAGCAGAAGCCTGACCAGAGCCCGA |
| | AACTGCTCATCAAATATGCCTCCCAGTCGTTTTCTGGCGTGCCCTCTCGCTTTTCCGGAAGCGGATCTGG |
| | CACAGACTTCACCTTGACCATCAATAGCCTGGAAACTGAGGACGCCGCTACGTATTTCTGCCAGCAGTCC |
| | TCCAGTCTGCCTTGGACATTTGGTCAGGGAACGAAGGTGGAGATCAAGGGTTCAACATCAGGGAGCGGGA |
| | AACCGGGCTCTGGCGAGGGCTCAACAAAGGGACAAGTGCAACTGCAAGAATCGGGACCCGGGCTGGTTAA |
| | ACCAAGTGAAACCCTTTCCCTCACTTGTACCGTAAGCGGCGGTAGCGTGTCCTCTGGTAGCTACTATTGG |
| | AGTTGGATTAGGCAGGCGCCAGGGAAAGGCCTCGAATGGATTGGGTATATCTACTACAGCGGCAGTAACT |
| | ACTACAACCCATCATTGAAGTCTAGAGTGACAATTAGTGTCGATACCTCTAAAAATCAATTCTCACTTAA |
| | GCTGCGAGCTGTAACCGCCGCAGACACTGCGGTGTACTATTGTGCCCGTTGGATGACCACTATCAAGGGC |
| | TACTTCGATTATTGGGGACAAGGGACATTAGTTACGGTGTCCTCCGCAACCAcgacgccagcgccgcgac |
| | caccaacaccggcgcccaccatcgcgtcgcagcccctgtccctgcgcccTgaggcgtgcttcatgtacgt |
| | ggcggcggccgcctttgtgcttctgttcttcgtgggctgcggggtgctgctgtcccgtaaacgCagacgt |
| | caacacggtcaactgtggtttccagaaggttttaaggtctccgaagcaagtaagaagaaaagacgtgaac |
| | cactgggagaagatagcgtcggtctgaaaccactcaagaatgccatggtttctaaactgagccagctgca |
| | gacggagctcctggcggccctgctggagtcagggctgagcaaagaggcactgctccaggcactgggCgag |
| | ccggggccctacctcctggctggagaaggccccctggacaagggggagtcctgcggcggcggtcgagggg |
| | agctggctgagctgcccaatgggctgggggagactcggggctccgaggacgagacCgacgacgatgggga |
| | agacttcacgccacccatcctcaaagagctggagaacctcagccctgaggaggcggcccaccagaaagcc |
| | gtggtggagacccttctgcaggaggacccgtggcgtgtggcgaagatggtcaagtcctacctgcagcagc |
| | acaacatcccacagcgggaggtggtcgataccactggcctcaaccagtcccacctgtcccaacacctcaa |
| | caagggcactcccatgaagacgcagaagcgggccgccctgtacacctggtaTgtccgcaagcagcgagag |
| | gtggcgcagcagttcacccatgcagggcagggagggctgattgaAgaGcccacaggAgatgagctaccaa |
| | ccaagaaggggcggaggaaccgtttcaagtggggcccagcatcccagcagatcctgttccaggcctatga |
| | gaggcagaagaaccctagcaaggaggagcgagaAacgctagtggaggagtgcaatagggcggaatgcatc |
| | cagagaggTgtgtcAccatcacaAgcacaAggTctgggctccaacctcgtcacggaggtgcgtgtctaca |
| | actggtttgccaaccggcgcaaagaagaagccttccggcacaagctggccatgacctgcagggatgagtt |
| | tcccaccatggtgtttccttctgggcagatcagccaggcctcggccttggccccggcccctccccaagtc |
| | ctgccccaggctccagcccctgcccctgctccagccatggtatcagctctggcccaggccccagcccctg |
| | tcccagtcctagccccaggccctcctcaAgctgtggccccacctgcccccaagcccacccaAgctgggga |
| | aggaacgctgtcagaggccctgctgcagctgcagtttgatgatgaagacctgggggccttgcttggcaac |
| | agcacagacccagctgtgttcacagacctggcatccgtcgacaactccgagtttcagcagctgctgaacc |
| | agggcatacctgtggccccccacacaactgagcccatgctgatggagtaccctgaggctataactcgcct |
| | agtgacaggggcccagaggccccccgacccagctcctgctccactgggggccccggggctccccaatggc |
| | ctcctttcaggagatgaagacttctcctccattgcggacatggacttctcagccctgctgagtcagatca |
| | gctcc |
| WPRE | aatcaacctctggattacaaaatttgtgaaagattgactggtattcttaactatgttgctccttttacgc |
| element | tatgtggatacgctgctttaatgcctttgtatcatgctattgcttcccgtatggctttcattttctcctc |
| SEQ ID | cttgtataaatcctggttgctgtctctttatgaggagttgtggcccgttgtcaggcaacgtggcgtggtC |
| NO: 1243 | tgcactgtgtttgctgacgcaacccccactggttggggcattgccaccacctgtcagctcctttccggga |
| | ctttcgctttccccctccctattgccacggcggaactcatcgccgcctgccttgcccgctgctggacagg |
| | ggctcggctgttgggcactgacaattccgtggtgttgtcggggaaatcatcgtcctttccttggctgctc |
| | gcctgtgttgccacctggattctgcgcgggacgtccttctgctacgtcccttcggccctcaatccagcgg |
| | accttccttcccgcggcctgctgccggctctgcggcctcttccgcgtcttcgccttcgccctcagacgag |
| | tcggatctccctttgggccgcctccccgcctg |

Table 19 provides a summary of the elements in each exemplary logic gate and their nucleotide position as compared to the Logic Gate sequences provided in SEQ ID NOs: 1120, 1121, 1122, 1123, and 1124. In some embodiments, the system comprises one or more element provided in Table 19.

In some embodiments, the logic gate DNA insert does not comprise the first 480 nucleotides or the last 473 nucleotides of SEQ ID NOs: 1120, 1121, 1122, 1123, or 1124. For example, in some embodiments, the logic gate DNA insert comprises the sequence as set forth in SEQ ID NOs: 1238, 1239, 1240, 1241, or 1242. In some embodiments, the system is encoded by a nucleic acid comprising a sequence with at least 80%, 85%, 90%, 91%, 92%, 93%, 94%, 95%, 96%, 97%, 98%, 99%, or 100% identity to a sequence comprising nucleotides 481-7257 of SEQ ID NO: 1120; a sequence with at least 80%, 85%, 90%, 91%, 92%, 93%, 94%, 95%, 96%, 97%, 98%, 99%, or 100% identity to a sequence comprising nucleotides 481-7239 of SEQ ID NO: 1121; a sequence with at least 80%, 85%, 90%, 91%, 92%, 93%, 94%, 95%, 96%, 97%, 98%, 99%, or 100% identity to a sequence comprising nucleotides 481-7621 of SEQ ID NO: 1122; a sequence with at least 80%, 85%, 90%, 91%, 92%, 93%, 94%, 95%, 96%, 97%, 98%, 99%, or 100% identity to a sequence comprising nucleotides 481-7636 of SEQ ID NO: 1123; or a sequence with at least 80%, 85%, 90%, 91%, 92%, 93%, 94%, 95%, 96%, 97%, 98%, 99%, 100% identity to a sequence comprising nucleotides 481-7621 of SEQ ID NO: 1124; a sequence with at least 80%, 85%, 90%, 91%, 92%, 93%, 94%, 95%, 96%, 97%, 98%, 99%, or 100% identity to a sequence comprising nucleotides 24-7707 of SEQ ID NO: 1120; a sequence with at least 80%, 85%, 90%, 91%, 92%, 93%, 94%, 95%, 96%, 97%, 98%, 99%, or 100% identity to a sequence comprising nucleotides 24-7689 of SEQ ID NO: 1121; a sequence with at least 80%, 85%, 90%, 91%, 92%, 93%, 94%, 95%, 96%, 97%, 98%, 99%, or 100% identity to a sequence comprising nucleotides 24-8071 of SEQ ID NO: 1122; a sequence with at least 80%, 85%, 90%, 91%, 92%, 93%, 94%, 95%, 96%, 97%, 98%, 99%, or 100% identity to a sequence comprising nucleotides 24-8086 of SEQ ID NO: 1123; or a sequence with at least 80%, 85%, 90%, 91%, 92%, 93%, 94%, 95%, 96%, 97%, 98%, 99%, or 100% identity to a sequence comprising nucleotides 24-8071 of SEQ ID NO: 1124.

TABLE 19
Nucleotide positions of elements in Exemplary Logic Gate Systems with Homology Regions

| | Logic Gate | Logic Gate | Logic Gate | Logic Gate | Logic Gate |
|---|---|---|---|---|---|
| | 39 SEQ ID | 43 SEQ ID | 47 SEQ ID | 219 SEQ ID | 239 SEQ ID |
| Element | NO: 1122 | NO: 1123 | NO: 1124 | NO: 1120 | NO: 1121 |
| sgRNA_94 SEQ ID NO: 932 | 1-20 | 1-20 | 1-20 | 1-20 | 1-20 |
| PAM sequence GGG | 21-23 | 21-23 | 21-23 | 21-23 | 21-23 |
| GS94 5′ homology SEQ ID NO: 1235 | 24-473 | 24-473 | 24-473 | 24-473 | 24-473 |
| BGH polyA SEQ ID NO: 995 | 481-708 | 481-708 | 481-708 | 481-708 | 481-708 |
| 6X HNF1α response elements SEQ | 736-845 | 736-845 | 736-845 | 736-845 | 736-845 |
| ID NO: 1245 | | | | | |
| YB-TATA promoter SEQ ID NO: 1246 | 851-875 | 851-875 | 851-875 | 851-875 | 851-875 |
| CD8a signal peptide (amino acid | 923-985 | 923-985 | 923-985 | 923-985 | 923-985 |
| SEQ ID NO: 825) | | | | | |
| CAR scFv | 986-1714 | 986-1729 | 986-1714 | 986-1729 | 986-1714 |
| AAA linker (amino acids AAA) | 1715-1723 | 1730-1738 | 1715-1723 | 1730-1738 | 1715-1723 |
| CD8a hinge (amino acid SEQ ID NO: 821) | 1724-1858 | 1739-1873 | 1724-1858 | 1739-1873 | 1724-1858 |
| CD8a TM (amino acid SEQ ID NO: 822 | 1859-1930 | 1874-1845 | 1859-1930 | 1874-1945 | 1859-1930 |
| 4-1BB (amino acid SEQ ID NO: 823) | 1931-2056 | 1946-2071 | 1931-2056 | 1946-2071 | 1931-2056 |
| CD3zeta (amino acid SEQ ID NO: 824) | 2057-2392 | 2072-2407 | 2057-2392 | 2072-2407 | 2057-2392 |
| WPRE SEQ ID NO: 1243 | 2396-2987 | 2411-3002 | 2396-2987 | 2411-3002 | 2396-2987 |
| SV40 polyA SEQ ID NO: 1244 | 3006-3137 | 3021-3152 | 3006-3137 | 3021-3152 | 3006-3137 |
| EF1a promoter SEQ ID NO: 991 | 3212-3585 | 3227-3600 | 3212-3585 | 3227-3600 | 3212-3585 |
| FAS/PTPN/TGFBR2 shRNA module | 3586-4604 | 3601-4619 | 3586-4604 | 3601-4619 | 3586-4604 |
| SEQ ID NO: 972 | | | | | |
| FAS/PTPN/TGFBR2 shRNA module | 3586-4580 | 3601-4595 | 3586-4580 | 3601-4595 | 3586-4580 |
| intron sequence (SEQ ID NO: 1252) | | | | | |
| CD8a signal peptide (amino acid | 4625-4687 | 4640-4702 | 4625-4687 | 4640-4702 | 4625-4687 |
| SEQ ID NO: 825) | | | | | |
| Priming receptor scFv | 4688-5425 | 4703-5440 | 4688-5425 | 4703-5443 | 4688-5425 |
| CD8a hinge (amino acid SEQ ID NO: 827) | 5429-5509 | 5444-5524 | 5429-5509 | 5447-5527 | 5429-5509 |
| Notch_TMD (amino acid SEQ ID NO: 828) | 5510-5575 | 5525-5590 | 5510-5575 | 5528-5593 | 5510-5575 |
| Notch1_JMD (amino acid SEQ ID NO: 829) | 5576-5704 | 5591-5719 | 5576-5704 | 5594-5722 | 5576-5704 |
| HNF1α DBD (amino acid SEQ ID NO: 830) | 5705-6553 | 5720-6568 | 5705-6553 | 5723-6571 | 5705-6553 |
| TCR Linker (amino acids TCR) | 6554-6562 | 6569-6577 | 6554-6562 | 6572-6580 | 6554-6562 |
| p65 (amino acid SEQ ID NO: 831) | 6563-7135 | 6578-7150 | 6563-7135 | 6581-7153 | 6563-7135 |
| human GH1 polyA SEQ ID NO: 994 | 7143-7621 | 7158-7636 | 7143-7621 | NA | NA |
| 2X Synthetic polyA SEQ ID NO: 993 | NA | NA | NA | 7161-7257 | 7143-7239 |
| GS94 3′ homology SEQ ID NO: 1236 | 7622-8071 | 7637-8086 | 7622-8071 | 7258-7707 | 7240-7689 |
| PAM sequence CCC | 8072-8074 | 8087-8089 | 8072-8074 | 7708-7710 | 7690-7692 |
| sgRNA 94 reverse complement sequence | 8075-8094 | 8090-8109 | 8075-8094 | 7711-7730 | 7693-7712 |
| SEQ ID NO: 1237 | | | | | |

### Suppressors of Gene Expression

In various embodiments, a logic gate system provided herein comprises one or more suppressors of gene expression. A suppressor of gene expression can be used, for example, to suppress activity of genes that have inhibitory effects on T cell properties, such as expansion or target cell killing. Suppressors of gene expression can function via any mechanism known in the art. Suppressors of gene expression can function, for example, by knock-out of the genomic sequence, suppression of gene transcription, or suppression of protein translation (“knock-down”). Examples of suppressors of gene expression include, but are not limited to, sgRNAs, shRNAs, RNAi molecules, TALENs, and zinc-finger nucleases (ZFNs).

In one aspect, provided herein are one or more nucleic acids, wherein the one or more nucleic acids encode: a first chimeric polypeptide that comprises a priming receptor comprising a first extracellular antigen-binding domain that specifically binds human Solute Carrier Family 34 Member 2 (SLC34A2); a second chimeric polypeptide that comprises a chimeric antigen receptor (CAR) comprising a second extracellular antigen-binding domain that specifically binds to human Transmembrane protease, serine 4 (TMPRSS4) and at least one or more nucleic acids comprising a nucleic acid that is complementary to a portion of a nucleic acid encoding human Fas Cell Surface Death Receptor (FAS) comprising the sequence set forth in SEQ ID NO: 964; and/or a nucleic that is complementary to a portion of the nucleic acid encoding human Transforming Growth factor (TGF)-β Receptor 2 (TGFBR2) comprising the sequence set forth in SEQ ID NO: 965. In some embodiments, the nucleic acid sequence is at least 15 nucleotides in length and is complementary to nucleotides 1126 to 1364 of the nucleic acid encoding human FAS set forth in SEQ ID NO: 964. In some embodiments, the nucleic acid sequence is complementary to nucleotides 1126 to 1147 of a nucleic acid encoding human FAS set forth in SEQ ID NO: 964. In some embodiments, the nucleic acid comprises a nucleic acid sequence at least 15 nucleotides in length complementary to a portion thereof of the nucleotide sequence encoding Phosphatase Non-Receptor Type 2 (PTPN2) set forth in SEQ ID NO: 966. In some embodiments, the nucleic acid sequence is complementary to nucleotides 518-559 of the nucleic acid encoding human PTPN2 set forth in SEQ ID NO: 966. In some embodiments, the nucleic acid sequence is complementary to nucleotides 518-539 of the nucleic acid encoding human PTPN2 set forth in SEQ ID NO: 966.

### RNA Interference Molecules

Transforming Growth Factor Beta Receptor 1 (TGF-βR1 or TGFBR1; HGNC: 11772, NCBI Entrez Gene: 7046, UniProtKB/Swiss-Prot: P36897) is a transmembrane serine/threonine protein kinase and forms a heteromeric complex with TGF-beta receptor type II (TGFRB2) when bound to TGF-beta, transducing the TGF-beta signal from the cell surface to the cytoplasm.

Transforming Growth Factor Beta Receptor 2 (TGF-βR2 or TGFBR2; HGNC: 11773, NCBI Entrez Gene: 7048, UniProtKB/Swiss-Prot: P37173) is a transmembrane serine/threonine protein kinase and forms a heterodimeric complex with TGF-beta receptor type-1 (TGFBR1) when bound to TGF-beta, resulting in transduction of the TGF-beta signal from the cell surface to the cytoplasm.

Fas Cell Surface Death Receptor (or Fas Receptor, FAS, CD95, or TNFRSF6; HGNC: 11920, NCBI Entrez Gene: 355; UniProtKB/Swiss-Prot: P25445) is an apoptosis-inducing TNF receptor superfamily member.

Protein Tyrosine Phosphatase Non-Receptor Type 2 (PTPN2; HGNC: 9650, NCBI Entrez Gene: 5771; UniProtKB/Swiss-Prot: P17706) is a phosphatase that regulates interferon and many other signaling pathways.

Sequences of FAS, PTPN2, and TGFBR2 nucleic acids, and nucleic acids that target such sequences are provided in Table 20 below. In some embodiments, the logic gate comprises at least one sequence as set forth in SEQ ID NOs: 967-990.

TABLE 20
Gene Expression Suppressors and Targets

| SEQ | | |
|---|---|---|
| ID | | |
| NO | Name | Sequence |
| 964 | FAS mRNA | CTCTTCTCCCGCGGGTTGGTGGACCCGCTCAGTACGGAGTTGGGGAAGCTCTTTCACTTC |
| | NCBI | GGAGGATTGCTCAACAACCATGCTGGGCATCTGGACCCTCCTACCTCTGGTTCTTACGTC |
| | NM_000043.6 | TGTTGCTAGATTATCGTCCAAAAGTGTTAATGCCCAAGTGACTGACATCAACTCCAAGGG |
| | | ATTGGAATTGAGGAAGACTGTTACTACAGTTGAGACTCAGAACTTGGAAGGCCTGCATCA |
| | | TGATGGCCAATTCTGCCATAAGCCCTGTCCTCCAGGTGAAAGGAAAGCTAGGGACTGCAC |
| | | AGTCAATGGGGATGAACCAGACTGCGTGCCCTGCCAAGAAGGGAAGGAGTACACAGACAA |
| | | AGCCCATTTTTCTTCCAAATGCAGAAGATGTAGATTGTGTGATGAAGGACATGGCTTAGA |
| | | AGTGGAAATAAACTGCACCCGGACCCAGAATACCAAGTGCAGATGTAAACCAAACTTTTT |
| | | TTGTAACTCTACTGTATGTGAACACTGTGACCCTTGCACCAAATGTGAACATGGAATCAT |
| | | CAAGGAATGCACACTCACCAGCAACACCAAGTGCAAAGAGGAAGGATCCAGATCTAACTT |
| | | GGGGTGGCTTTGTCTTCTTCTTTTGCCAATTCCACTAATTGTTTGGGTGAAGAGAAAGGA |
| | | AGTACAGAAAACATGCAGAAAGCACAGAAAGGAAAACCAAGGTTCTCATGAATCTCCAAC |
| | | TTTAAATCCTGAAACAGTGGCAATAAATTTATCTGATGTTGACTTGAGTAAATATATCAC |
| | | CACTATTGCTGGAGTCATGACACTAAGTCAAGTTAAAGGCTTTGTTCGAAAGAATGGTGT |
| | | CAATGAAGCCAAAATAGATGAGATCAAGAATGACAATGTCCAAGACACAGCAGAACAGAA |
| | | AGTTCAACTGCTTCGTAATTGGCATCAACTTCATGGAAAGAAAGAAGCGTATGACACATT |
| | | GATTAAAGATCTCAAAAAAGCCAATCTTTGTACTCTTGCAGAGAAAATTCAGACTATCAT |
| | | CCTCAAGGACATTACTAGTGACTCAGAAAATTCAAACTTCAGAAATGAAATCCAAAGCTT |
| | | GGTCTAGAGTGAAAAACAACAAATTCAGTTCTGAGTATATGCAATTAGTGTTTGAAAAGA |
| | | TTCTTAATAGCTGGCTGTAAATACTGCTTGGTTTTTTACTGGGTACATTTTATCATTTAT |
| | | TAGCGCTGAAGAGCCAACATATTTGTAGATTTTTAATATCTCATGATTCTGCCTCCAAGG |
| | | ATGTTTAAAATCTAGTTGGGAAAACAAACTTCATCAAGAGTAAATGCAGTGGCATGCTAA |
| | | GTACCCAAATAGGAGTGTATGCAGAGGATGAAAGATTAAGATTATGCTCTGGCATCTAAC |
| | | ATATGATTCTGTAGTATGAATGTAATCAGTGTATGTTAGTACAAATGTCTATCCACAGGC |
| | | TAACCCCACTCTATGAATCAATAGAAGAAGCTATGACCTTTTGCTGAAATATCAGTTACT |
| | | GAACAGGCAGGCCACTTTGCCTCTAAATTACCTCTGATAATTCTAGAGATTTTACCATAT |
| | | TTCTAAACTTTGTTTATAACTCTGAGAAGATCATATTTATGTAAAGTATATGTATTTGAG |
| | | TGCAGAATTTAAATAAGGCTCTACCTCAAAGACCTTTGCACAGTTTATTGGTGTCATATT |
| | | ATACAATATTTCAATTGTGAATTCACATAGAAAACATTAAATTATAATGTTTGACTATTA |
| | | TATATGTGTATGCATTTTACTGGCTCAAAACTACCTACTTCTTTCTCAGGCATCAAAAGC |
| | | ATTTTGAGCAGGAGAGTATTACTAGAGCTTTGCCACCTCTCCATTTTTGCCTTGGTGCTC |
| | | ATCTTAATGGCCTAATGCACCCCCAAACATGGAAATATCACCAAAAAATACTTAATAGTC |
| | | CACCAAAAGGCAAGACTGCCCTTAGAAATTCTAGCCTGGTTTGGAGATACTAACTGCTCT |
| | | CAGAGAAAGTAGCTTTGTGACATGTCATGAACCCATGTTTGCAATCAAAGATGATAAAAT |
| | | AGATTCTTATTTTTCCCCCACCCCCGAAAATGTTCAATAATGTCCCATGTAAAACCTGCT |
| | | ACAAATGGCAGCTTATACATAGCAATGGTAAAATCATCATCTGGATTTAGGAATTGCTCT |
| | | TGTCATACCCCCAAGTTTCTAAGATTTAAGATTCTCCTTACTACTATCCTACGTTTAAAT |
| | | ATCTTTGAAAGTTTGTATTAAATGTGAATTTTAAGAAATAATATTTATATTTCTGTAAAT |
| | | GTAAACTGTGAAGATAGTTATAAACTGAAGCAGATACCTGGAACCACCTAAAGAACTTCC |
| | | ATTTATGGAGGATTTTTTTGCCCCTTGTGTTTGGAATTATAAAATATAGGTAAAAGTACG |
| | | TAATTAAATAATGTTTTTGGTATTTCTGGTTTTCTCTTTTTTGGTAGGGGCTTGCTTTTT |
| | | GGTTTTGTCTTCCTTTTCTCTAACTGATGCTAAATATAACTTGTCTTTAATGCTTCTTGG |
| | | ATCCCTTAGAAGGTACTTCCTTTTTAACCTTAACCCTTTTAGTAGTTAAATAATTATTTC |
| | | CATAGGTTGCTATTGCCAAGAAGACCTCTTCCAAACAGCACATGATTATTCGTCAAACAG |
| | | TTTCGTATTCCAGATACTGGAATGTGGATAAGAAAGTATACATTTCAAGGGGTAGGTTTT |
| | | ATTATTAAGAAAGCCAAATGAGGATTTTGAAATATTCTTTCCTGCATATTATCCATTCTA |
| | | GCTACATGCTGGCCAGTGGGCCACCTTTCTTTTCTGCAATTTAATGCTAGTAATATATTC |
| | | TATTTAACCCATGAGTCCCAAAGTATTAGCATTTCAACATGTAAGCATGTCGGTAAGATA |
| | | GTTGTGCTTTGCTTAGGGTTCCCTCCTGTGTTATGGTCTGGAAAGTGTCTTTAGGCAGAA |
| | | AGTCTGAGTGATCACAGGGTTCACTCATTAATTTCTCTTTTCTGAGCCATCATAGTCTGT |
| | | GCTGTCTGCTCTCCAGTTTTCTATTTCTAGACAGAAGTAGGGCAAGTTAGGTACTAGTTA |
| | | TTCTTCATGGCCAGAAGTGCAAGTTCTACTTTGCAAGACAAGATTAAGTTAGAGAACACC |
| | | CTATTCCACTTTGGTGAACTCAGAGCAAGAACTTTGAGTTCCTTTGGGAGGAAGACAGTG |
| | | GAGAAGTCTTTGTACTTGGTGATGTGGTTTTTTTCCTCATGGCTTCACCTAGTGGCCCCA |
| | | AGCATGACTTCTCCCATGTCAATGAGCACAGCCACATTCCCGAGTTGAGGTGACCCCACG |
| | | GTCCAGAATCATCCTCATTCTGGTGAACCTGGTTCTCTTTGTGGTGGGCATACTGGGTAG |
| | | GAGAATCACCCAAAGGTCACCCATGAGCTGCAGAAAAAAAGGCTATTTGCAGAAGGAGCT |
| | | CACAGATCACATTGAAAGCATTGCATATTCAAACATCTTGGTCTTCTTTATTGGCATGCC |
| | | CACAGGGTCTTCTGACCTCTGATTAGATCAGACACTTTTTAGATATTGAATCATCAGTTT |
| | | CTGTACAACTATCTGAATAAGGTATATAATCAATGAAATTTAGAATTTTTTTCTATGCTT |
| | | ACTCCTGATTGGTAATTTGTTTGGGTTTAGAATTCTATACAAGGCCATTTGTAATTTTCC |
| | | TCAGCACTTTAAAAATATTAAACCATGTTTTCTTAA |
| 965 | TGF-β | ACTCGCGCGCACGGAGCGACGACACCCCCGCGCGTGCACCCGCTCGGGACAGGAGCCGGA |
| | Receptor 2 | CTCCTGTGCAGCTTCCCTCGGCCGCCGGGGGCCTCCCCGCGCCTCGCCGGCCTCCAGGCC |
| | (TGFBR2) | CCCTCCTGGCTGGCGAGCGGGCGCCACATCTGGCCCGCACATCTGCGCTGCCGGCCCGGC |
| | cDNA | GCGGGGTCCGGAGAGGGCGCGGCGCGGAGGCGCAGCCAGGGGTCCGGGAAGGCGCCGTCC |
| | | GCTGCGCTGGGGGCTCGGTCTATGACGAGCAGCGGGGTCTGCCATGGGTCGGGGGCTGCT |
| | | CAGGGGCCTGTGGCCGCTGCACATCGTCCTGTGGACGCGTATCGCCAGCACGATCCCACC |
| | | GCACGTTCAGAAGTCGGTTAATAACGACATGATAGTCACTGACAACAACGGTGCAGTCAA |
| | | GTTTCCACAACTGTGTAAATTTTGTGATGTGAGATTTTCCACCTGTGACAACCAGAAATC |
| | | CTGCATGAGCAACTGCAGCATCACCTCCATCTGTGAGAAGCCACAGGAAGTCTGTGTGGC |
| | | TGTATGGAGAAAGAATGACGAGAACATAACACTAGAGACAGTTTGCCATGACCCCAAGCT |
| | | CCCCTACCATGACTTTATTCTGGAAGATGCTGCTTCTCCAAAGTGCATTATGAAGGaaaa |
| | | aaaaaaGCCTGGTGAGACTTTCTTCATGTGTTCCTGTAGCTCTGATGAGTGCAATGACAA |
| | | CATCATCTTCTCAGAAGAATATAACACCAGCAATCCTGACTTGTTGCTAGTCATATTTCA |
| | | AGTGACAGGCATCAGCCTCCTGCCACCACTGGGAGTTGCCATATCTGTCATCATCATCTT |
| | | CTACTGCTACCGCGTTAACCGGCAGCAGAAGCTGAGTTCAACCTGGGAAACCGGCAAGAC |
| | | GCGGAAGCTCATGGAGTTCAGCGAGCACTGTGCCATCATCCTGGAAGATGACCGCTCTGA |
| | | CATCAGCTCCACGTGTGCCAACAACATCAACCACAACACAGAGCTGCTGCCCATTGAGCT |
| | | GGACACCCTGGTGGGGAAAGGTCGCTTTGCTGAGGTCTATAAGGCCAAGCTGAAGCAGAA |
| | | CACTTCAGAGCAGTTTGAGACAGTGGCAGTCAAGATCTTTCCCTATGAGGAGTATGCCTC |
| | | TTGGAAGACAGAGAAGGACATCTTCTCAGACATCAATCTGAAGCATGAGAACATACTCCA |
| | | GTTCCTGACGGCTGAGGAGCGGAAGACGGAGTTGGGGAAACAATACTGGCTGATCACCGC |
| | | CTTCCACGCCAAGGGCAACCTACAGGAGTACCTGACGCGGCATGTCATCAGCTGGGAGGA |
| | | CCTGCGCAAGCTGGGCAGCTCCCTCGCCCGGGGGATTGCTCACCTCCACAGTGATCACAC |
| | | TCCATGTGGGAGGCCCAAGATGCCCATCGTGCACAGGGACCTCAAGAGCTCCAATATCCT |
| | | CGTGAAGAACGACCTAACCTGCTGCCTGTGTGACTTTGGGCTTTCCCTGCGTCTGGACCC |
| | | TACTCTGTCTGTGGATGACCTGGCTAACAGTGGGCAGGTGGGAACTGCAAGATACATGGC |
| | | TCCAGAAGTCCTAGAATCCAGGATGAATTTGGAGAATGTTGAGTCCTTCAAGCAGACCGA |
| | | TGTCTACTCCATGGCTCTGGTGCTCTGGGAAATGACATCTCGCTGTAATGCAGTGGGAGA |
| | | AGTAAAAGATTATGAGCCTCCATTTGGTTCCAAGGTGCGGGAGCACCCCTGTGTCGAAAG |
| | | CATGAAGGACAACGTGTTGAGAGATCGAGGGCGACCAGAAATTCCCAGCTTCTGGCTCAA |
| | | CCACCAGGGCATCCAGATGGTGTGTGAGACGTTGACTGAGTGCTGGGACCACGACCCAGA |
| | | GGCCCGTCTCACAGCCCAGTGTGTGGCAGAACGCTTCAGTGAGCTGGAGCATCTGGACAG |
| | | GCTCTCGGGGAGGAGCTGCTCGGAGGAGAAGATTCCTGAAGACGGCTCCCTAAACACTAC |
| | | CAAATAGCTCTTCTGGGGCAGGCTGGGCCATGTCCAAAGAGGCTGCCCCTCTCACCAAAG |
| | | AACAGAGGCAGCAGGAAGCTGCCCCTGAACTGATGCTTCCTGGAAAACCAAGGGGGTCAC |
| | | TCCCCTCCCTGTAAGCTGTGGGGATAAGCAGAAACAACAGCAGCAGGGAGTGGGTGACAT |
| | | AGAGCATTCTATGCCTTTGACATTGTCATAGGATAAGCTGTGTTAGCACTTCCTCAGGAA |
| | | ATGAGATTGATTTTTACAATAGCCAATAACATTTGCACTTTATTAATGCCTGtatataaa |
| | | tatgaatagctatgttttatatatatatatatatatctatatatgtctatagctctatat |
| | | atataGCCATACCTTGAAAAGAGACAAGGAAAAACATCAAATATTCCCAGGAAATTGGTT |
| | | TTATTGGAGAACTCCAGAACCAAGCAGAGAAGGAAGGGACCCATGACAGCATTAGCATTT |
| | | GACAATCACACATGCAGTGGTTCTCTGACTGTAAAACAGTGAACTTTGCATGAGGAAAGA |
| | | GGCTCCATGTCTCACAGCCAGCTATGACCACATTGCACTTGCTTTTGCAAAATAATCATT |
| | | CCCTGCCTAGCACTTCTCTTCTGGCCATGGAACTAAGTACAGTGGCACTGTTTGAGGACC |
| | | AGTGTTCCCGGGGTTCCTGTGTGCCCTTATTTCTCCTGGACTTTTCATTTAAGCTCCAAG |
| | | CCCCAAATCTGGGGGGCTAGTTTAGAAACTCTCCCTCAACCTAGTTTAGAAACTCTACCC |
| | | CATCTTTAATACCTTGAATGTTTTGAACCCCACTTTTTACCTTCATGGGTTGCAGAAAAA |
| | | TCAGAACAGATGTCCCCATCCATGCGATTGCCCCACCATCTACTAATGAAAAATTGTTCT |
| | | TTTTTTCATCTTTCCCCTGCACTTATGTTACTATTCTCTGCTCCCAGCCTTCATCCTTTT |
| | | CTAAAAAGGAGCAAATTCTCACTCTAGGCTTTATCGTGTTTACTTTTTCATTACACTTGA |
| | | CTTGATTTTCTAGTTTTCTATACAAACACCAATGGGTTCCATCTTTCTGGGCTCCTGATT |
| | | GCTCAAGCACAGTTTGGCCTGATGAAGAGGATTTCAACTACACAATACTATCATTGTCAG |
| | | GACTATGACCTCAGGCACTCTAAACATAtgttttgtttggtcagcacagcgtttcaaaaa |
| | | gtgaagccactttataaatatttggagattttgcaggaaaatctggatccccagGTAAGG |
| | | ATAGCAGATGGTTTTCAGTTATCTCCAGTCCACGTTCACAAAATGTGAAGGTGTGGAGAC |
| | | ACTTACAAAGCTGCCTCACTTCTCACTGTAAACATTAGCTCTTTCCACTGCCTACCTGGA |
| | | CCCCAGTCTAGGAATTAAATCTGCACCTAACCAAGGTCCCTTGTAAGAAATGTCCATTCA |
| | | AGCAGTCATTCTCTGGGTATATAATATGATTTTGACTACCTTATCTGGTGTTAAGATTTG |
| | | AAGTTGGCCTTTTATTGGACTAAAGGGGAACTCCTTTAAGGGTCTCAGTTAGCCCAAGTT |
| | | TCTTTTGCTTATATGTTAATAGTTTTACCCTCTGCATTGGAGAGAGGAGTGCTTTACTCC |
| | | AAGAAGCTTTCCTCATGGTTACCGTTCTCTCCATCATGCCAGCCTTCTCAACCTTTGCAG |
| | | AAATTACTAGAGAGGATTTGAATGTGGGACACAAAGGTCCCATTTGCAGTTAGAAAATTT |
| | | GTGTCCACAAGGACAAGAACAAAGTATGAGCTTTAAAACTCCATAGGAAACTTGTTAATC |
| | | AACAAAGAAGTGTTAATGCTGCAAGTAATCTCTTTTTTAAAACTTTTTGAAGCTACTTAT |
| | | TTTCAGCCAAATAGGAATATTAGAGAGGGACTGGTAGTGAGAATATCAGCTCTGTTTGGA |
| | | TGGTGGAAGGTCTCATTTTATTGAGATTTTTAAGATACATGCAAAGGTTTGGAAATAGAA |
| | | CCTCTAGGCACCCTCCTCAGTGTGGGTGGGCTGAGAGTTAAAGACAGTGTGGCTGCAGTA |
| | | GCATAGAGGCGCCTAGAAATTCCACTTGCACCGTAGGGCATGCTGATACCATCCCAATAG |
| | | CTGTTGCCCATTGACCTCTAGTGGTGAGTTTCTAGAATACTGGTCCATTCATGAGATATT |
| | | CAAGATTCAAGAGTATTCTCACTTCTGGGTTATCAGCATAAACTGGAATGTAGTGTCAGA |
| | | GGATACTGTGGCTTGTTTTGTTTATGtttttttttCTTATTCAAGAAAAAAGACCAAGGA |
| | | ATAACATTCTGTAGTTCCTAAAAATACTGACTTTTTTCACTACTATACATAAAGGGAAAG |
| | | TTTTATTCTTTTATGGAACACTTCAGCTGTACTCATGTATTAAAATAGGAATGTGAATGC |
| | | TATATACTCTTTTTATATCAAAAGTCTCAAGCACTTATTTTTATTCTATGCATTGTTTGT |
| | | CTTTTACATAAATAAAATGTTTATTAGATTGAATAAAGCAAAATACTCAGGTGAGCATCC |
| | | TGCCTCCTGTTCCCATTCCTAGTAGCTAAA |
| 966 | PTPN2 | GCATGCGCCGCAGCGCCAGCGCTCTCCCCGGATCGTGCGGGGCCTGAGCCTCTCCGCCGG |
| | mRNA NCBI | CGCAGGCTCTGCTCGCGCCAGCTCGCTCCCGCAGCCATGCCCACCACCATCGAGCGGGAG |
| | NM_002828.4 | TTCGAAGAGTTGGATACTCAGCGTCGCTGGCAGCCGCTGTACTTGGAAATTCGAAATGAG |
| | | TCCCATGACTATCCTCATAGAGTGGCCAAGTTTCCAGAAAACAGAAATCGAAACAGATAC |
| | | AGAGATGTAAGCCCATATGATCACAGTCGTGTTAAACTGCAAAATGCTGAGAATGATTAT |
| | | ATTAATGCCAGTTTAGTTGACATAGAAGAGGCACAAAGGAGTTACATCTTAACACAGGGT |
| | | CCACTTCCTAACACATGCTGCCATTTCTGGCTTATGGTTTGGCAGCAGAAGACCAAAGCA |
| | | GTTGTCATGCTGAACCGCATTGTGGAGAAAGAATCGGTTAAATGTGCACAGTACTGGCCA |
| | | ACAGATGACCAAGAGATGCTGTTTAAAGAAACAGGATTCAGTGTGAAGCTCTTGTCAGAA |
| | | GATGTGAAGTCGTATTATACAGTACATCTACTACAATTAGAAAATATCAATAGTGGTGAA |
| | | ACCAGAACAATATCTCACTTTCATTATACTACCTGGCCAGATTTTGGAGTCCCTGAATCA |
| | | CCAGCTTCATTTCTCAATTTCTTGTTTAAAGTGAGAGAATCTGGCTCCTTGAACCCTGAC |
| | | CATGGGCCTGCGGTGATCCACTGTAGTGCAGGCATTGGGCGCTCTGGCACCTTCTCTCTG |
| | | GTAGACACTTGTCTTGTTTTGATGGAAAAAGGAGATGATATTAACATAAAACAAGTGTTA |
| | | CTGAACATGAGAAAATACCGAATGGGTCTTATTCAGACCCCAGATCAACTGAGATTCTCA |
| | | TACATGGCTATAATAGAAGGAGCAAAATGTATAAAGGGAGATTCTAGTATACAGAAACGA |
| | | TGGAAAGAACTTTCTAAGGAAGACTTATCTCCTGCCTTTGATCATTCACCAAACAAAATA |
| | | ATGACTGAAAAATACAATGGGAACAGAATAGGTCTAGAAGAAGAAAAACTGACAGGTGAC |
| | | CGATGTACAGGACTTTCCTCTAAAATGCAAGATACAATGGAGGAGAACAGTGAGAGTGCT |
| | | CTACGGAAACGTATTCGAGAGGACAGAAAGGCCACCACAGCTCAGAAGGTGCAGCAGATG |
| | | AAACAGAGGCTAAATGAGAATGAACGAAAAAGAAAAAGGTGGTTATATTGGCAACCTATT |
| | | CTCACTAAGATGGGGTTTATGTCAGTCATTTTGGTTGGCGCTTTTGTTGGCTGGACACTG |
| | | TTTTTTCAGCAAAATGCCCTATAAACAATTAATTTTGCCCAGCAAGCTTCTGCACTAGTA |
| | | ACTGACAGTGCTACATTAATCATAGGGGTTTGTCTGCAGCAAACGCCTCATATCCCAAAA |
| | | ACGGTGCAGTAGAATAGACATCAACCAGATAAGTGATATTTACAGTCACAAGCCCAACAT |
| | | CTCAGGACTCTTGACTGCAGGTTCCTCTGAACCCCAAACTGTAAATGGCTGTCTAAAATA |
| | | AAGACATTCATGTTTGTTAAAAACTGGTAAATTTTGCAACTGTATTCATACATGTCAAAC |
| | | ACAGTATTTCACCTGACCAACATTGAGATATCCTTTATCACAGGATTTGTTTTTGGAGGC |
| | | TATCTGGATTTTAACCTGCACTTGATATAAGCAATAAATATTGTGGTTTTATCTACGTTA |
| | | TTGGAAAGAAAATGACATTTAAATAATGTGTGTAATGTATAATGTACTATTGACATGGGC |
| | | ATCAACACTTTTATTCTTAAGCATTTCAGGGTAAATATATTTTATAAGTATCTATTTAAT |
| | | CTTTTGTAGTTAACTGTACTTTTTAAGAGCTCAATTTGAAAAATCTGTTACTAAAAAAAT |
| | | AAATTGTATGTCGATTGAATTGTACTGGATACATTTTCCATTTTTCTAAAGAGAAGTTTG |
| | | ATATGAGCAGTTAGAAGTTGGAATAAGCAATTTCTACTATATATTGCATTTCTTTTATGT |
| | | TTTACAGTTTTCCCCATTTTAAAAAGAAAAGCAAACAAAGAAACAAAAGTTTTTCCTAAA |
| | | AATATCTTTGAAGGAAAATTCTCCTTACTGGGATAGTCAGGTAAACAGTTGGTCAAGACT |
| | | TTGTAAAGAAATTGGTTTCTGTAAATCCCATTATTGATATGTTTATTTTTCATGAAAATT |
| | | TCAATGTAGTTGGGGTAGATTATGATTTAGGAAGCAAAAGTAAGAAGCAGCATTTTATGA |
| | | TTCATAATTTCAGTTTACTAGACTGAAGTTTTGAAGTAAACACTTTTCAGTTTCTTTCTA |
| | | CTTCAATAAATAGTATGATTATATGCAAACCTTACATTGTCATTTTAACTTAATGAATAT |
| | | TTTTTAAAGCAAACTGTTTAATGAATTTAACTGCTCATTTGAATGCTAGCTTTCCTCAGA |
| | | TTTCAACATTCCATTCAGTGTTTAATTTGTCTTACTTAAACTTGAAATTGTTGTTACAAA |
| | | TTTAATTGCTAGGAGGCATGGATAGCATACATTATTATGGATAGCATACCTTATTTCAGT |
| | | GGTTTTCAAACTATGCTCATTGGATGTCCAGGTGGGTCAAGAGGTTACTTTCAACCACAG |
| | | CATCTCTGCCTTGTCTCTTTATATGCCACATAAGATTTCTGCATAAGGCTTAAGTATTTT |
| | | AAAGGGGGCAGTTATCATTTAAAAACAGTTTGGTCGGGCGCGGTGGCTCATGCCTGTAAT |
| | | CCCAGCACTTTGGGAGGCTGAAGTGGGCAGATCACCTGAGGTCAGGAGTTCAAGACCAGC |
| | | GAGGGCACCTGTAATCTCAGCTACTCAGGAGGCTGAGGTAGGAGAATTGCTTGAACCCAG |
| | | CTGGCCAACGTGGTGAAACACCATCTCTACTAAAAATGCAAAAATTAGCTGGGCATGGTG |
| | | GAGGGCACCTGTAATCTCAGCTACTCAGGAGGCTGAGGTAGGAGAATTGCTTGAACCCAG |
| | | GAGATGGAGGTTGCAGTGAGCTGAGATCACGTCACTGCACTCCAGCCAGGGCGACAGAGC |
| | | GAGACTCCATCTCAAAAGAAACAAACAAAAAAAACAGTTTGGGCCGGGTGTGGTGGCTCA |
| | | CGCTTGTAATCCCAGCACTTCGGAAGGCCAAGGCGGGCGGATCACGAGGTCAAGAGATGG |
| | | AGACTGTCCTGGCCAACATGGTGAAATCCCTTCTTTACTAAAAATACAAAAATTATCTGG |
| | | GCGTGGTGGTGCATGCCTGTAGTCCCAGCTCCTTGGGAGGCTAAGGCAGGAGAATCACTT |
| | | GAACCCGGGAGGCAGAGGTTGCAGTGAGCCGAGATTGCACCACTGCACTCCAGCCTGGCA |
| | | ACAGAGCAAGACTTCGTCTCAAAAAAAAAAAAAAAAAAAGTTTGAAAACCATTGGTATAG |
| | | ATAGATATTTTGAATTGATTTGCATAGTCTCCTTGAATGTGTTAAATTATGTTGAAAGTA |
| | | TGAAAGCAGGATGTAGGTGGTACTACATATTAAATAAGATTTATATAACA |
| 967 | FAS_11 | TTAAGAATCTTTTCAAACACTA |
| 968 | PTPN2_14 | TCTGACAAGAGCTTCACACTGA |
| 969 | TGFBR2_37 | TTATGTAAAAGACAAACAATGC |
| 970 | TGFBR2_23 | TAAAAATCAATCTCATTTCCTG |
| 971 | FAS/TGFBR2/ | gtaagtcgactcgttggatccccactacccggatcaacgccctaggtttatgtttggatg |
| | TGFBR2 | aactgacatacgcgtatccgtcttaagaatcttttcaaacactagtagtgaaatatatat |
| | shRNA | taaactagtgtttgaaaagattcttattacggtaacgcggaattcgcaactattttatca |
| | module | attttttgcgtcgacgacggtgacttaggagtatgccggatcaacgccctaggtttatgt |
| | | ttggatgaactgacatacgcgtatccgtctaaaaatcaatctcatttcctggtagtgaaa |
| | | tatatattaaaccaggaaatgagattgatttttttacggtaacgcggaattcgcaactat |
| | | tttatcaattttttgcgtcgacgactgtgacagcagagtatgccggatcaacgccctagg |
| | | tttatgtttggatgaactgacatacgcgtatccgtcttatgtaaaagacaaacaatgcgt |
| | | agtgaaatatatattaaacgcattgtttgtcttttacatattacggtaacgcggaattcg |
| | | caactattttatcaattttttgcgtcgaccggaactatcttgaagagtagtagtggacta |
| | | gtgtgacgctgctgacccctttctttcccttctacaga |
| 972 | FAS/PTPN2/ | gtaaGTcgactcgttggatccCCACTACCCGGATCAACGCCCTAGGTTTATGTTTGGATG |
| | TGFBR2/ | AACTGACATACGCGTATCCGTCTTAAGAATCTTTTCAAACACTAGTAGTGAAATATATAT |
| | TGFBR2 | TAAACTAGTGTTTGAAAAGATTCTTATTACGGTAACGCGGAATTCGCAACTATTTTATCA |
| | shRNA | ATTTTTTGCGTCGACACTTCAAGGGGCTTGCGGCCGCAACCATCTCCATGGCTGTTTGAA |
| | module | TGAGGCTTCAGTACTTTACAGAATCGTTGCCTGCACATCTTGGAAACACTTGCTGGGATT |
| | | ACTTCGACTTCTTAACCCAACAGAAGGCTCGAGAAGGTATATTGCTGTTGACAGTGAGCG |
| | | CCAGTGTGAAGCTCTTGTCAGATAGTGAAGCCACAGATGTATCTGACAAGAGCTTCACAC |
| | | TGATGCCTACTGCCTCGGACTTCAAGGGGCTAGAATTCGAGCAATTATCTTGTTTACTAA |
| | | AACTGAATACCTTGCTATCTCTTTGATACATTTTTACAAAGCTGAATTAAAATGGTATAA |
| | | ATTAAATCACTTTGACGGTGACTTAGGAGTATGCCGGATCAACGCCCTAGGTTTATGTTT |
| | | GGATGAACTGACATACGCGTATCCGTCTAAAAATCAATCTCATTTCCTGGTAGTGAAATA |
| | | TATATTAAACCAGGAAATGAGATTGATTTTTTTACGGTAACGCGGAATTCGCAACTATTT |
| | | TATCAATTTTTTGCGTCGACGACTGTGACAGCAGAGTATGCCGGATCAACGCCCTAGGTT |
| | | TATGTTTGGATGAACTGACATACGCGTATCCGTCTTATGTAAAAGACAAACAATGCGTAG |
| | | TGAAATATATATTAAACGCATTGTTTGTCTTTTACATATTACGGTAACGCGGAATTCGCA |
| | | ACTATTTTATCAATTTTTTGCGTCGACCGGAACTATCTTGAAGAGTAGTAGTGGactagt |
| | | gtgacgctgctgacccctttctttcccttctACAGATCCAAGCTGTGACCGGCGCCTAC |
| 1252 | FAS/PTPN2/ | gtaaGTcgactcgttggatccCCACTACCCGGATCAACGCCCTAGGTTTATGTTTGGATG |
| | TGFBR2/ | AACTGACATACGCGTATCCGTCTTAAGAATCTTTTCAAACACTAGTAGTGAAATATATAT |
| | TGFBR2 | TAAACTAGTGTTTGAAAAGATTCTTATTACGGTAACGCGGAATTCGCAACTATTTTATCA |
| | shRNA | ATTTTTTGCGTCGACACTTCAAGGGGCTTGCGGCCGCAACCATCTCCATGGCTGTTTGAA |
| | module | TGAGGCTTCAGTACTTTACAGAATCGTTGCCTGCACATCTTGGAAACACTTGCTGGGATT |
| | | ACTTCGACTTCTTAACCCAACAGAAGGCTCGAGAAGGTATATTGCTGTTGACAGTGAGCG |
| | | CCAGTGTGAAGCTCTTGTCAGATAGTGAAGCCACAGATGTATCTGACAAGAGCTTCACAC |
| | | TGATGCCTACTGCCTCGGACTTCAAGGGGCTAGAATTCGAGCAATTATCTTGTTTACTAA |
| | | AACTGAATACCTTGCTATCTCTTTGATACATTTTTACAAAGCTGAATTAAAATGGTATAA |
| | | ATTAAATCACTTTGACGGTGACTTAGGAGTATGCCGGATCAACGCCCTAGGTTTATGTTT |
| | | GGATGAACTGACATACGCGTATCCGTCTAAAAATCAATCTCATTTCCTGGTAGTGAAATA |
| | | TATATTAAACCAGGAAATGAGATTGATTTTTTTACGGTAACGCGGAATTCGCAACTATTT |
| | | TATCAATTTTTTGCGTCGACGACTGTGACAGCAGAGTATGCCGGATCAACGCCCTAGGTT |
| | | TATGTTTGGATGAACTGACATACGCGTATCCGTCTTATGTAAAAGACAAACAATGCGTAG |
| | | TGAAATATATATTAAACGCATTGTTTGTCTTTTACATATTACGGTAACGCGGAATTCGCA |
| | | ACTATTTTATCAATTTTTTGCGTCGACCGGAACTATCTTGAAGAGTAGTAGTGGactagt |
| | | gtgacgctgctgaccccttttttcccttctACAG |
| 973 | FAS miR3G- | GTAAGTCGACTCGTTGGATCCCCACTACCCGGATCAACGCCCTAGGTTTATGTTTGGATG |
| | PTPN2 | AACTGACATACGCGTATCCGTCTTAAGAATCTTTTCAAACACTAGTAGTGAAATATATAT |
| | miRE | TAAACTAGTGTTTGAAAAGATTCTTATTACGGTAACGCGGAATTCGCAACTATTTTATCA |
| | Module | ATTTTTTGCGTCGACACTTCAAGGGGCTTGCGGCCGCAACCATCTCCATGGCTGTTTGAA |
| | | TGAGGCTTCAGTACTTTACAGAATCGTTGCCTGCACATCTTGGAAACACTTGCTGGGATT |
| | | ACTTCGACTTCTTAACCCAACAGAAGGCTCGAGAAGGTATATTGCTGTTGACAGTGAGCG |
| | | CCAGTGTGAAGCTCTTGTCAGATAGTGAAGCCACAGATGTATCTGACAAGAGCTTCACAC |
| | | TGATGCCTACTGCCTCGGACTTCAAGGGGCTAGAATTCGAGCAATTATCTTGTTTACTAA |
| | | AACTGAATACCTTGCTATCTCTTTGATACATTTTTACAAAGCTGAATTAAAATGGTATAA |
| | | ATTAAATCACTTTTTCATCTGACCAGTAGTGGACTAGTGTGACGCTGCTGACCCCTTTCT |
| | | TTCCCTTCTACAG |
| 974 | 2XTGFBR2- | CCGGATCAACGCCCTAGGTTTATGTTTGGATGAACTGACATACGCGTATCCGTCTAAAAA |
| | miR3G | TCAATCTCATTTCCTGGTAGTGAAATATATATTAAACCAGGAAATGAGATTGATTTTTTT |
| | Module | ACGGTAACGCGGAATTCGCAACTATTTTATCAATTTTTTGCGTCGACGACTGTGACAGCA |
| | | GAGTATGCCGGATCAACGCCCTAGGTTTATGTTTGGATGAACTGACATACGCGTATCCGT |
| | | CTTATGTAAAAGACAAACAATGCGTAGTGAAATATATATTAAACGCATTGTTTGTCTTTT |
| | | ACATATTACGGTAACGCGGAATTCGCAACTATTTTATCAATTTTTTGCGTCGAC |
| 975 | FAS-PTPN2- | CCACTACCCGGATCAACGCCCTAGGTTTATGTTTGGATGAACTGACATACGCGTATCCGT |
| | 2XTGFBR2 | CTTAAGAATCTTTTCAAACACTAGTAGTGAAATATATATTAAACTAGTGTTTGAAAAGAT |
| | miR3G | TCTTATTACGGTAACGCGGAATTCGCAACTATTTTATCAATTTTTTGCGTCGACACTTCA |
| | Module | AGGGGCTTGCGGCCGCAACCATCTCCATGGCTGTTTGAATGAGGCTTCAGTACTTTACAG |
| | | AATCGTTGCCTGCACATCTTGGAAACACTTGCTGGGATTACTTCGACTTCTTAACCCAAC |
| | | AGAAGGCTCGAGAAGGTATATTGCTGTTGACAGTGAGCGCCAGTGTGAAGCTCTTGTCAG |
| | | ATAGTGAAGCCACAGATGTATCTGACAAGAGCTTCACACTGATGCCTACTGCCTCGGACT |
| | | TCAAGGGGCTAGAATTCGAGCAATTATCTTGTTTACTAAAACTGAATACCTTGCTATCTC |
| | | TTTGATACATTTTTACAAAGCTGAATTAAAATGGTATAAATTAAATCACTTTGACGGTGA |
| | | CTTAGGAGTATGCCGGATCAACGCCCTAGGTTTATGTTTGGATGAACTGACATACGCGTA |
| | | TCCGTCTAAAAATCAATCTCATTTCCTGGTAGTGAAATATATATTAAACCAGGAAATGAG |
| | | ATTGATTTTTTTACGGTAACGCGGAATTCGCAACTATTTTATCAATTTTTTGCGTCGACG |
| | | ACTGTGACAGCAGAGTATGCCGGATCAACGCCCTAGGTTTATGTTTGGATGAACTGACAT |
| | | ACGCGTATCCGTCTTATGTAAAAGACAAACAATGCGTAGTGAAATATATATTAAACGCAT |
| | | TGTTTGTCTTTTACATATTACGGTAACGCGGAATTCGCAACTATTTTATCAATTTTTTGC |
| | | GTCGAC |
| 976 | FAS- | GGATCTGCGATCGCTCCGGTGCCCGTCAGTGGGCAGAGCGCACATCGCCCACAGTCCCCG |
| | TGFBR2- | AGAAGTTGGGGGGAGGGGTCGGCAATTGAACGGGTGCCTAGAGAAGGTGGCGCGGGGTAA |
| | TGFBR2 | ACTGGGAAAGTGATGTCGTGTACTGGCTCCGCCTTTTTCCCGAGGGTGGGGGAGAACCGT |
| | architecture 1 | ATATAAGTGCAGTAGTCGCCGTGAACGTTCTTTTTCGCAACGGGTTTGCCGCCAGAACAC |
| | (3G-3G-3G) | AGCTGAAGCTTCGAGGGGCTCGCATCTCTCCTTCACGCGCCCGCCGCCCTACCTGAGGCC |
| | triple shRNA | GCCATCCACGCCGGTTGAGTCGCGTTCTGCCGCCTCCCGCCTGTGGTGCCTCCTGAACTG |
| | module, with | CGTCCGCCGTCTAGGTAAGTCGACTCGTTGGATCCCCACTACCCGGATCAACGCCCTAGG |
| | potential | TTTATGTTTGGATGAACTGACATACGCGTATCCGTCTTAAGAATCTTTTCAAACACTAGT |
| | transgene | AGTGAAATATATATTAAACTAGTGTTTGAAAAGATTCTTATTACGGTAACGCGGAATTCG |
| | | CAACTATTTTATCAATTTTTTGCGTCGACGACGGTGACTTAGGAGTATGCCGGATCAACG |
| | | CCCTAGGTTTATGTTTGGATGAACTGACATACGCGTATCCGTCTAAAAATCAATCTCATT |
| | | TCCTGGTAGTGAAATATATATTAAACCAGGAAATGAGATTGATTTTTTTACGGTAACGCG |
| | | GAATTCGCAACTATTTTATCAATTTTTTGCGTCGACGACTGTGACAGCAGAGTATGCCGG |
| | | ATCAACGCCCTAGGTTTATGTTTGGATGAACTGACATACGCGTATCCGTCTTATGTAAAA |
| | | GACAAACAATGCGTAGTGAAATATATATTAAACGCATTGTTTGTCTTTTACATATTACGG |
| | | TAACGCGGAATTCGCAACTATTTTATCAATTTTTTGCGTCGACCGGAACTATCTTGAAGA |
| | | GTAGTAGTGGACTAGTGTGACGCTGCTGACCCCTTTCTTTCCCTTCTACAGATCCAAGCT |
| | | GTGACCGGCGCCTACACCTGCAGCCCAAGCTTACCNNNNNNNNNNNNNNNNNNNNNNNNN |
| | | NNNNNTAAAGGACGGGTGGCATCCCTGTGACCCCTCCCCAGTGCCTCTCCTGGCCCTGGA |
| | | AGTTGCCACTCCAGTGCCCACCAGCCTTGTCCTAATAAAATTAAGTTGCATCATTTTGTC |
| | | TGACTAGGTGTCCTTCTATAATATTATGGGGTGGAGGGGGGTGGTATGGAGCAAGGGGCA |
| | | AGTTGGGAAGACAACCTGTAGGGCCTGCGGGGTCTATTGGGAACCAAGCTGGAGTGCAGT |
| | | GGCACAATCTTGGCTCACTGCAATCTCCGCCTCCTGGGTTCAAGCGATTCTCCTGCCTCA |
| | | GCCTCCCGAGTTGTTGGGATTCCAGGCATGCATGACCAGGCTCAGCTAATTTTTGTTTTT |
| | | TTGGTAGAAACGGGGTTTCACCATATTGGCCAGGCTGGTCTCCAACTCCTAATCTCAGGT |
| | | GATCTACCCACCTTGGCCTCCCAAATTGCTGGGATTACAGGCGTGAACCACTGCTCCCTT |
| | | CCCTGTCCTTC |
| | | (NNNNNNNNNNNNNNNNNNNNNNNNNNNNNN can be absent or can encode |
| | | a transgene of any length) |
| 977 | FAS- | GGATCTGCGATCGCTCCGGTGCCCGTCAGTGGGCAGAGCGCACATCGCCCACAGTCCCCG |
| | TGFBR2- | AGAAGTTGGGGGGAGGGGTCGGCAATTGAACGGGTGCCTAGAGAAGGTGGCGCGGGGTAA |
| | TGFBR2 | ACTGGGAAAGTGATGTCGTGTACTGGCTCCGCCTTTTTCCCGAGGGTGGGGGAGAACCGT |
| | architecture 1 | ATATAAGTGCAGTAGTCGCCGTGAACGTTCTTTTTCGCAACGGGTTTGCCGCCAGAACAC |
| | (3G-3G-3G) | AGCTGAAGCTTCGAGGGGCTCGCATCTCTCCTTCACGCGCCCGCCGCCCTACCTGAGGCC |
| | triple shRNA | GCCATCCACGCCGGTTGAGTCGCGTTCTGCCGCCTCCCGCCTGTGGTGCCTCCTGAACTG |
| | module, 5′ | CGTCCGCCGTCTAGGTAAGTCGACTCGTTGGATCCCCACTACCCGGATCAACGCCCTAGG |
| | end | TTTATGTTTGGATGAACTGACATACGCGTATCCGTCTTAAGAATCTTTTCAAACACTAGT |
| | | AGTGAAATATATATTAAACTAGTGTTTGAAAAGATTCTTATTACGGTAACGCGGAATTCG |
| | | CAACTATTTTATCAATTTTTTGCGTCGACGACGGTGACTTAGGAGTATGCCGGATCAACG |
| | | CCCTAGGTTTATGTTTGGATGAACTGACATACGCGTATCCGTCTAAAAATCAATCTCATT |
| | | TCCTGGTAGTGAAATATATATTAAACCAGGAAATGAGATTGATTTTTTTACGGTAACGCG |
| | | GAATTCGCAACTATTTTATCAATTTTTTGCGTCGACGACTGTGACAGCAGAGTATGCCGG |
| | | ATCAACGCCCTAGGTTTATGTTTGGATGAACTGACATACGCGTATCCGTCTTATGTAAAA |
| | | GACAAACAATGCGTAGTGAAATATATATTAAACGCATTGTTTGTCTTTTACATATTACGG |
| | | TAACGCGGAATTCGCAACTATTTTATCAATTTTTTGCGTCGACCGGAACTATCTTGAAGA |
| | | GTAGTAGTGGACTAGTGTGACGCTGCTGACCCCTTTCTTTCCCTTCTACAGATCCAAGCT |
| | | GTGACCGGCGCCTACACCTGCAGCCCAAGCTTACC |
| 978 | FAS- | TAAAGGACGGGTGGCATCCCTGTGACCCCTCCCCAGTGCCTCTCCTGGCCCTGGAAGTTG |
| | TGFBR2- | CCACTCCAGTGCCCACCAGCCTTGTCCTAATAAAATTAAGTTGCATCATTTTGTCTGACT |
| | TGFBR2 | AGGTGTCCTTCTATAATATTATGGGGTGGAGGGGGGTGGTATGGAGCAAGGGGCAAGTTG |
| | architecture 1 | GGAAGACAACCTGTAGGGCCTGCGGGGTCTATTGGGAACCAAGCTGGAGTGCAGTGGCAC |
| | (3G-3G-3G) | AATCTTGGCTCACTGCAATCTCCGCCTCCTGGGTTCAAGCGATTCTCCTGCCTCAGCCTC |
| | triple shRNA | CCGAGTTGTTGGGATTCCAGGCATGCATGACCAGGCTCAGCTAATTTTTGTTTTTTTGGT |
| | module, 3′ | AGAAACGGGGTTTCACCATATTGGCCAGGCTGGTCTCCAACTCCTAATCTCAGGTGATCT |
| | end | ACCCACCTTGGCCTCCCAAATTGCTGGGATTACAGGCGTGAACCACTGCTCCCTTCCCTG |
| | | TCCTTC |
| 979 | TGFBR2- | GGATCTGCGATCGCTCCGGTGCCCGTCAGTGGGCAGAGCGCACATCGCCCACAGTCCCCG |
| | TGFBR2- | AGAAGTTGGGGGGAGGGGTCGGCAATTGAACGGGTGCCTAGAGAAGGTGGCGCGGGGTAA |
| | FAS | ACTGGGAAAGTGATGTCGTGTACTGGCTCCGCCTTTTTCCCGAGGGTGGGGGAGAACCGT |
| | architecture 2 | ATATAAGTGCAGTAGTCGCCGTGAACGTTCTTTTTCGCAACGGGTTTGCCGCCAGAACAC |
| | triple shRNA | AGCTGAAGCTTCGAGGGGCTCGCATCTCTCCTTCACGCGCCCGCCGCCCTACCTGAGGCC |
| | module, with | GCCATCCACGCCGGTTGAGTCGCGTTCTGCCGCCTCCCGCCTGTGGTGCCTCCTGAACTG |
| | potential | CGTCCGCCGTCTAGGTAAGTCGACTCGTTGGATCCCCACTACCCGGATCAACGCCCTAGG |
| | transgene | TTTATGTTTGGATGAACTGACATACGCGTATCCGTCTAAAAATCAATCTCATTTCCTGGT |
| | insert | AGTGAAATATATATTAAACCAGGAAATGAGATTGATTTTTTTACGGTAACGCGGAATTCG |
| | | CAACTATTTTATCAATTTTTTGCGTCGACACTTCAAGGGGCTTGCGGCCGCAACCATCTC |
| | | CATGGCGACGGTGACTTAGGAGTATGCCGGATCAACGCCCTAGGTTTATGTTTGGATGAA |
| | | CTGACATACGCGTATCCGTCTTATGTAAAAGACAAACAATGCGTAGTGAAATATATATTA |
| | | AACGCATTGTTTGTCTTTTACATATTACGGTAACGCGGAATTCGCAACTATTTTATCAAT |
| | | TTTTTGCGTCGACGACTGTGACAGCAGAGTATGCCGGATCAACGCCCTAGGTTTATGTTT |
| | | GGATGAACTGACATACGCGTATCCGTCTTAAGAATCTTTTCAAACACTAGTAGTGAAATA |
| | | TATATTAAACTAGTGTTTGAAAAGATTCTTATTACGGTAACGCGGAATTCGCAACTATTT |
| | | TATCAATTTTTTGCGTCGACCGGAACTATCTTGAAGAGTAGTAGTGGACTAGTGTGACGC |
| | | TGCTGACCCCTTTCTTTCCCTTCTACAGATCCAAGCTGTGACCGGCGCCTACACCTGCAG |
| | | CCCAAGCTTACCNNNNNNNNNNNNNNNNNNNNNNNNNNNNNNTAAAGGACGGGTGGCATC |
| | | CCTGTGACCCCTCCCCAGTGCCTCTCCTGGCCCTGGAAGTTGCCACTCCAGTGCCCACCA |
| | | GCCTTGTCCTAATAAAATTAAGTTGCATCATTTTGTCTGACTAGGTGTCCTTCTATAATA |
| | | TTATGGGGTGGAGGGGGGTGGTATGGAGCAAGGGGCAAGTTGGGAAGACAACCTGTAGGG |
| | | CCTGCGGGGTCTATTGGGAACCAAGCTGGAGTGCAGTGGCACAATCTTGGCTCACTGCAA |
| | | TCTCCGCCTCCTGGGTTCAAGCGATTCTCCTGCCTCAGCCTCCCGAGTTGTTGGGATTCC |
| | | AGGCATGCATGACCAGGCTCAGCTAATTTTTGTTTTTTTGGTAGAAACGGGGTTTCACCA |
| | | TATTGGCCAGGCTGGTCTCCAACTCCTAATCTCAGGTGATCTACCCACCTTGGCCTCCCA |
| | | AATTGCTGGGATTACAGGCGTGAACCACTGCTCCCTTCCCTGTCCTTC |
| | | (NNNNNNNNNNNNNNNNNNNNNNNNNNNNNN can be absent or can encode |
| | | a transgene of any length) |
| 980 | TGFBR2- | GGATCTGCGATCGCTCCGGTGCCCGTCAGTGGGCAGAGCGCACATCGCCCACAGTCCCCG |
| | TGFBR2- | AGAAGTTGGGGGGAGGGGTCGGCAATTGAACGGGTGCCTAGAGAAGGTGGCGCGGGGTAA |
| | FAS | ACTGGGAAAGTGATGTCGTGTACTGGCTCCGCCTTTTTCCCGAGGGTGGGGGAGAACCGT |
| | architecture 2 | ATATAAGTGCAGTAGTCGCCGTGAACGTTCTTTTTCGCAACGGGTTTGCCGCCAGAACAC |
| | triple shRNA | AGCTGAAGCTTCGAGGGGCTCGCATCTCTCCTTCACGCGCCCGCCGCCCTACCTGAGGCC |
| | module, 5′ | GCCATCCACGCCGGTTGAGTCGCGTTCTGCCGCCTCCCGCCTGTGGTGCCTCCTGAACTG |
| | end | CGTCCGCCGTCTAGGTAAGTCGACTCGTTGGATCCCCACTACCCGGATCAACGCCCTAGG |
| | | TTTATGTTTGGATGAACTGACATACGCGTATCCGTCTAAAAATCAATCTCATTTCCTGGT |
| | | AGTGAAATATATATTAAACCAGGAAATGAGATTGATTTTTTTACGGTAACGCGGAATTCG |
| | | CAACTATTTTATCAATTTTTTGCGTCGACACTTCAAGGGGCTTGCGGCCGCAACCATCTC |
| | | CATGGCGACGGTGACTTAGGAGTATGCCGGATCAACGCCCTAGGTTTATGTTTGGATGAA |
| | | CTGACATACGCGTATCCGTCTTATGTAAAAGACAAACAATGCGTAGTGAAATATATATTA |
| | | AACGCATTGTTTGTCTTTTACATATTACGGTAACGCGGAATTCGCAACTATTTTATCAAT |
| | | TTTTTGCGTCGACGACTGTGACAGCAGAGTATGCCGGATCAACGCCCTAGGTTTATGTTT |
| | | GGATGAACTGACATACGCGTATCCGTCTTAAGAATCTTTTCAAACACTAGTAGTGAAATA |
| | | TATATTAAACTAGTGTTTGAAAAGATTCTTATTACGGTAACGCGGAATTCGCAACTATTT |
| | | TATCAATTTTTTGCGTCGACCGGAACTATCTTGAAGAGTAGTAGTGGACTAGTGTGACGC |
| | | TGCTGACCCCTTTCTTTCCCTTCTACAGATCCAAGCTGTGACCGGCGCCTACACCTGCAG |
| | | CCCAAGCTTACC |
| 981 | TGFBR2- | TAAAGGACGGGTGGCATCCCTGTGACCCCTCCCCAGTGCCTCTCCTGGCCCTGGAAGTTG |
| | TGFBR2- | CCACTCCAGTGCCCACCAGCCTTGTCCTAATAAAATTAAGTTGCATCATTTTGTCTGACT |
| | FAS | AGGTGTCCTTCTATAATATTATGGGGTGGAGGGGGGTGGTATGGAGCAAGGGGCAAGTTG |
| | architecture 2 | GGAAGACAACCTGTAGGGCCTGCGGGGTCTATTGGGAACCAAGCTGGAGTGCAGTGGCAC |
| | triple shRNA | AATCTTGGCTCACTGCAATCTCCGCCTCCTGGGTTCAAGCGATTCTCCTGCCTCAGCCTC |
| | module, 3′ | CCGAGTTGTTGGGATTCCAGGCATGCATGACCAGGCTCAGCTAATTTTTGTTTTTTTGGT |
| | end | AGAAACGGGGTTTCACCATATTGGCCAGGCTGGTCTCCAACTCCTAATCTCAGGTGATCT |
| | | ACCCACCTTGGCCTCCCAAATTGCTGGGATTACAGGCGTGAACCACTGCTCCCTTCCCTG |
| | | TCCTTC |
| 982 | FAS- | GGATCTGCGATCGCTCCGGTGCCCGTCAGTGGGCAGAGCGCACATCGCCCACAGTCCCCG |
| | TGFBR2- | AGAAGTTGGGGGGAGGGGTCGGCAATTGAACGGGTGCCTAGAGAAGGTGGCGCGGGGTAA |
| | TGFBR2 | ACTGGGAAAGTGATGTCGTGTACTGGCTCCGCCTTTTTCCCGAGGGTGGGGGAGAACCGT |
| | architecture 3 | ATATAAGTGCAGTAGTCGCCGTGAACGTTCTTTTTCGCAACGGGTTTGCCGCCAGAACAC |
| | (3G-E-3G) | AGCTGAAGCTTCGAGGGGCTCGCATCTCTCCTTCACGCGCCCGCCGCCCTACCTGAGGCC |
| | triple shRNA | GCCATCCACGCCGGTTGAGTCGCGTTCTGCCGCCTCCCGCCTGTGGTGCCTCCTGAACTG |
| | module, with | CGTCCGCCGTCTAGGTAAGTCGACTCGTTGGATCCCCACTACCCGGATCAACGCCCTAGG |
| | potential | TTTATGTTTGGATGAACTGACATACGCGTATCCGTCTTAAGAATCTTTTCAAACACTAGT |
| | transgene | AGTGAAATATATATTAAACTAGTGTTTGAAAAGATTCTTATTACGGTAACGCGGAATTCG |
| | insert | CAACTATTTTATCAATTTTTTGCGTCGACACTTCAAGGGGCTTGCGGCCGCAACCATCTC |
| | | CATGGCGACGGTGACTTAGGAGTATGTGTTTGAATGAGGCTTCAGTACTTTACAGAATCG |
| | | TTGCCTGCACATCTTGGAAACACTTGCTGGGATTACTTCGACTTCTTAACCCAACAGAAG |
| | | GCTCGAGAAGGTATATTGCTGTTGACAGTGAGCGCAGGAAATGAGATTGATTTTTTTAGT |
| | | GAAGCCACAGATGTATAAAAATCAATCTCATTTCCTGTGCCTACTGCCTCGGACTTCAAG |
| | | GGGCTAGAATTCGAGCAATTATCTTGTTTACTAAAACTGAATACCTTGCTATCTCTTTGA |
| | | TACATTTTTACAAAGCTGAATTAAAATGGTATAAATTAAATCACTTTGACTGTGACAGCA |
| | | GAGTATGCCGGATCAACGCCCTAGGTTTATGTTTGGATGAACTGACATACGCGTATCCGT |
| | | CTTATGTAAAAGACAAACAATGCGTAGTGAAATATATATTAAACGCATTGTTTGTCTTTT |
| | | ACATATTACGGTAACGCGGAATTCGCAACTATTTTATCAATTTTTTGCGTCGACCGGAAC |
| | | TATCTTGAAGAGTAGTAGTGGACTAGTGTGACGCTGCTGACCCCTTTCTTTCCCTTCTAC |
| | | AGATCCAAGCTGTGACCGGCGCCTACACCTGCAGCCCAAGCTTACCNNNNNNNNNNNNNN |
| | | NNNNNNNNNNNNNNNNTAAAGGACGGGTGGCATCCCTGTGACCCCTCCCCAGTGCCTCTC |
| | | CTGGCCCTGGAAGTTGCCACTCCAGTGCCCACCAGCCTTGTCCTAATAAAATTAAGTTGC |
| | | ATCATTTTGTCTGACTAGGTGTCCTTCTATAATATTATGGGGTGGAGGGGGGTGGTATGG |
| | | AGCAAGGGGCAAGTTGGGAAGACAACCTGTAGGGCCTGCGGGGTCTATTGGGAACCAAGC |
| | | TGGAGTGCAGTGGCACAATCTTGGCTCACTGCAATCTCCGCCTCCTGGGTTCAAGCGATT |
| | | CTCCTGCCTCAGCCTCCCGAGTTGTTGGGATTCCAGGCATGCATGACCAGGCTCAGCTAA |
| | | TTTTTGTTTTTTTGGTAGAAACGGGGTTTCACCATATTGGCCAGGCTGGTCTCCAACTCC |
| | | TAATCTCAGGTGATCTACCCACCTTGGCCTCCCAAATTGCTGGGATTACAGGCGTGAACC |
| | | ACTGCTCCCTTCCCTGTCCTTC |
| | | (NNNNNNNNNNNNNNNNNNNNNNNNNNNNNN can be absent or can encode |
| | | a transgene of any length) |
| 983 | FAS- | GGATCTGCGATCGCTCCGGTGCCCGTCAGTGGGCAGAGCGCACATCGCCCACAGTCCCCG |
| | TGFBR2- | AGAAGTTGGGGGGAGGGGTCGGCAATTGAACGGGTGCCTAGAGAAGGTGGCGCGGGGTAA |
| | TGFBR2 | ACTGGGAAAGTGATGTCGTGTACTGGCTCCGCCTTTTTCCCGAGGGTGGGGGAGAACCGT |
| | architecture 3 | ATATAAGTGCAGTAGTCGCCGTGAACGTTCTTTTTCGCAACGGGTTTGCCGCCAGAACAC |
| | (3G-E-3G) | AGCTGAAGCTTCGAGGGGCTCGCATCTCTCCTTCACGCGCCCGCCGCCCTACCTGAGGCC |
| | triple shRNA | GCCATCCACGCCGGTTGAGTCGCGTTCTGCCGCCTCCCGCCTGTGGTGCCTCCTGAACTG |
| | module, 5′ | CGTCCGCCGTCTAGGTAAGTCGACTCGTTGGATCCCCACTACCCGGATCAACGCCCTAGG |
| | end | TTTATGTTTGGATGAACTGACATACGCGTATCCGTCTTAAGAATCTTTTCAAACACTAGT |
| | | AGTGAAATATATATTAAACTAGTGTTTGAAAAGATTCTTATTACGGTAACGCGGAATTCG |
| | | CAACTATTTTATCAATTTTTTGCGTCGACACTTCAAGGGGCTTGCGGCCGCAACCATCTC |
| | | CATGGCGACGGTGACTTAGGAGTATGTGTTTGAATGAGGCTTCAGTACTTTACAGAATCG |
| | | TTGCCTGCACATCTTGGAAACACTTGCTGGGATTACTTCGACTTCTTAACCCAACAGAAG |
| | | GCTCGAGAAGGTATATTGCTGTTGACAGTGAGCGCAGGAAATGAGATTGATTTTTTTAGT |
| | | GAAGCCACAGATGTATAAAAATCAATCTCATTTCCTGTGCCTACTGCCTCGGACTTCAAG |
| | | GGGCTAGAATTCGAGCAATTATCTTGTTTACTAAAACTGAATACCTTGCTATCTCTTTGA |
| | | TACATTTTTACAAAGCTGAATTAAAATGGTATAAATTAAATCACTTTGACTGTGACAGCA |
| | | GAGTATGCCGGATCAACGCCCTAGGTTTATGTTTGGATGAACTGACATACGCGTATCCGT |
| | | CTTATGTAAAAGACAAACAATGCGTAGTGAAATATATATTAAACGCATTGTTTGTCTTTT |
| | | ACATATTACGGTAACGCGGAATTCGCAACTATTTTATCAATTTTTTGCGTCGACCGGAAC |
| | | TATCTTGAAGAGTAGTAGTGGACTAGTGTGACGCTGCTGACCCCTTTCTTTCCCTTCTAC |
| | | AGATCCAAGCTGTGACCGGCGCCTACACCTGCAGCCCAAGCTTACC |
| 984 | FAS- | TAAAGGACGGGTGGCATCCCTGTGACCCCTCCCCAGTGCCTCTCCTGGCCCTGGAAGTTG |
| | TGFBR2- | CCACTCCAGTGCCCACCAGCCTTGTCCTAATAAAATTAAGTTGCATCATTTTGTCTGACT |
| | TGFBR2 | AGGTGTCCTTCTATAATATTATGGGGTGGAGGGGGGTGGTATGGAGCAAGGGGCAAGTTG |
| | architecture 3 | GGAAGACAACCTGTAGGGCCTGCGGGGTCTATTGGGAACCAAGCTGGAGTGCAGTGGCAC |
| | (3G-E-3G) | AATCTTGGCTCACTGCAATCTCCGCCTCCTGGGTTCAAGCGATTCTCCTGCCTCAGCCTC |
| | triple shRNA | CCGAGTTGTTGGGATTCCAGGCATGCATGACCAGGCTCAGCTAATTTTTGTTTTTTTGGT |
| | module, 3′ | AGAAACGGGGTTTCACCATATTGGCCAGGCTGGTCTCCAACTCCTAATCTCAGGTGATCT |
| | end | ACCCACCTTGGCCTCCCAAATTGCTGGGATTACAGGCGTGAACCACTGCTCCCTTCCCTG |
| | | TCCTTC |
| 985 | FAS- | CGGATCAACGCCCTAGGTTTATGTTTGGATGAACTGACATACGCGTATCCGTCTTAAGAA |
| | TGFBR2- | TCTTTTCAAACACTAGTAGTGAAATATATATTAAACTAGTGTTTGAAAAGATTCTTATTA |
| | TGFBR2 | CGGTAACGCGGAATTCGCAACTATTTTATCAATTTTTTGCGTCGACACTTCAAGGGGCTT |
| | (3G-E-3G) | GCGGCCGCAACCATCTCCATGGCGACGGTGACTTAGGAGTATGTGTTTGAATGAGGCTTC |
| | triple shRNA | AGTACTTTACAGAATCGTTGCCTGCACATCTTGGAAACACTTGCTGGGATTACTTCGACT |
| | module | TCTTAACCCAACAGAAGGCTCGAGAAGGTATATTGCTGTTGACAGTGAGCGCAGGAAATG |
| | | AGATTGATTTTTTTAGTGAAGCCACAGATGTATAAAAATCAATCTCATTTCCTGTGCCTA |
| | | CTGCCTCGGACTTCAAGGGGCTAGAATTCGAGCAATTATCTTGTTTACTAAAACTGAATA |
| | | CCTTGCTATCTCTTTGATACATTTTTACAAAGCTGAATTAAAATGGTATAAATTAAATCA |
| | | CTTTGACTGTGACAGCAGAGTATGCCGGATCAACGCCCTAGGTTTATGTTTGGATGAACT |
| | | GACATACGCGTATCCGTCTTATGTAAAAGACAAACAATGCGTAGTGAAATATATATTAAA |
| | | CGCATTGTTTGTCTTTTACATATTACGGTAACGCGGAATTCGCAACTATTTTATCAATTT |
| | | TTTGCGTCGAC |
| 986 | TGFBR2_23- | TGTTTGAATGAGGCTTCAGTACTTTACAGAATCGTTGCCTGCACATCTTGGAAACACTTG |
| | TGBR2_37 | CTGGGATTACTTCGACTTCTTAACCCAACAGAAGGCTCGAGAAGGTATATTGCTGTTGAC |
| | miR3G and | AGTGAGCGCAGGAAATGAGATTGATTTTTTTAGTGAAGCCACAGATGTATAAAAATCAAT |
| | miR3E | CTCATTTCCTGTGCCTACTGCCTCGGACTTCAAGGGGCTAGAATTCGAGCAATTATCTTG |
| | Module | TTTACTAAAACTGAATACCTTGCTATCTCTTTGATACATTTTTACAAAGCTGAATTAAAA |
| | | TGGTATAAATTAAATCACTTTGACTGTGACAGCAGAGTATGCCGGATCAACGCCCTAGGT |
| | | TTATGTTTGGATGAACTGACATACGCGTATCCGTCTTATGTAAAAGACAAACAATGCGTA |
| | | GTGAAATATATATTAAACGCATTGTTTGTCTTTTACATATTACGGTAACGCGGAATTCGC |
| | | AACTATTTTATCAATTTTTTGCGTCGAC |
| 987 | TGFBR2- | CCGGATCAACGCCCTAGGTTTATGTTTGGATGAACTGACATACGCGTATCCGTCTAAAAA |
| | TGFBR2- | TCAATCTCATTTCCTGGTAGTGAAATATATATTAAACCAGGAAATGAGATTGATTTTTTT |
| | FAS miR3G | ACGGTAACGCGGAATTCGCAACTATTTTATCAATTTTTTGCGTCGACACTTCAAGGGGCT |
| | triple shRNA | TGCGGCCGCAACCATCTCCATGGCGACGGTGACTTAGGAGTATGCCGGATCAACGCCCTA |
| | module | GGTTTATGTTTGGATGAACTGACATACGCGTATCCGTCTTATGTAAAAGACAAACAATGC |
| | | GTAGTGAAATATATATTAAACGCATTGTTTGTCTTTTACATATTACGGTAACGCGGAATT |
| | | CGCAACTATTTTATCAATTTTTTGCGTCGACGACTGTGACAGCAGAGTATGCCGGATCAA |
| | | CGCCCTAGGTTTATGTTTGGATGAACTGACATACGCGTATCCGTCTTAAGAATCTTTTCA |
| | | AACACTAGTAGTGAAATATATATTAAACTAGTGTTTGAAAAGATTCTTATTACGGTAACG |
| | | CGGAATTCGCAACTATTTTATCAATTTTTTGCGTCGAC |
| 988 | FAS-PTPN2- | GGATCTGCGATCGCTCCGGTGCCCGTCAGTGGGCAGAGCGCACATCGCCCACAGTCCCCG |
| | TGFBR2- | AGAAGTTGGGGGGAGGGGTCGGCAATTGAACGGGTGCCTAGAGAAGGTGGCGCGGGGTAA |
| | TGFBR2 | ACTGGGAAAGTGATGTCGTGTACTGGCTCCGCCTTTTTCCCGAGGGTGGGGGAGAACCGT |
| | quadruple | ATATAAGTGCAGTAGTCGCCGTGAACGTTCTTTTTCGCAACGGGTTTGCCGCCAGAACAC |
| | shRNA | AGCTGAAGCTTCGAGGGGCTCGCATCTCTCCTTCACGCGCCCGCCGCCCTACCTGAGGCC |
| | module, with | GCCATCCACGCCGGTTGAGTCGCGTTCTGCCGCCTCCCGCCTGTGGTGCCTCCTGAACTG |
| | potential | CGTCCGCCGTCTAGGTAAGTCGACTCGTTGGATCCCCACTACCCGGATCAACGCCCTAGG |
| | transgene | TTTATGTTTGGATGAACTGACATACGCGTATCCGTCTTAAGAATCTTTTCAAACACTAGT |
| | insert | AGTGAAATATATATTAAACTAGTGTTTGAAAAGATTCTTATTACGGTAACGCGGAATTCG |
| | | CAACTATTTTATCAATTTTTTGCGTCGACACTTCAAGGGGCTTGCGGCCGCAACCATCTC |
| | | CATGGCTGTTTGAATGAGGCTTCAGTACTTTACAGAATCGTTGCCTGCACATCTTGGAAA |
| | | CACTTGCTGGGATTACTTCGACTTCTTAACCCAACAGAAGGCTCGAGAAGGTATATTGCT |
| | | GTTGACAGTGAGCGCCAGTGTGAAGCTCTTGTCAGATAGTGAAGCCACAGATGTATCTGA |
| | | CAAGAGCTTCACACTGATGCCTACTGCCTCGGACTTCAAGGGGCTAGAATTCGAGCAATT |
| | | ATCTTGTTTACTAAAACTGAATACCTTGCTATCTCTTTGATACATTTTTACAAAGCTGAA |
| | | TTAAAATGGTATAAATTAAATCACTTTGACGGTGACTTAGGAGTATGCCGGATCAACGCC |
| | | CTAGGTTTATGTTTGGATGAACTGACATACGCGTATCCGTCTAAAAATCAATCTCATTTC |
| | | CTGGTAGTGAAATATATATTAAACCAGGAAATGAGATTGATTTTTTTACGGTAACGCGGA |
| | | ATTCGCAACTATTTTATCAATTTTTTGCGTCGACGACTGTGACAGCAGAGTATGCCGGAT |
| | | CAACGCCCTAGGTTTATGTTTGGATGAACTGACATACGCGTATCCGTCTTATGTAAAAGA |
| | | CAAACAATGCGTAGTGAAATATATATTAAACGCATTGTTTGTCTTTTACATATTACGGTA |
| | | ACGCGGAATTCGCAACTATTTTATCAATTTTTTGCGTCGACCGGAACTATCTTGAAGAGT |
| | | AGTAGTGGACTAGTGTGACGCTGCTGACCCCTTTCTTTCCCTTCTACAGATCCAAGCTGT |
| | | GACCGGCGCCTACACCTGCAGCCCAAGCTTACCNNNNNNNNNNNNNNNNNNNNNNNNNNN |
| | | NNNTAAAGGACGGGTGGCATCCCTGTGACCCCTCCCCAGTGCCTCTCCTGGCCCTGGAAG |
| | | TTGCCACTCCAGTGCCCACCAGCCTTGTCCTAATAAAATTAAGTTGCATCATTTTGTCTG |
| | | ACTAGGTGTCCTTCTATAATATTATGGGGTGGAGGGGGGTGGTATGGAGCAAGGGGCAAG |
| | | TTGGGAAGACAACCTGTAGGGCCTGCGGGGTCTATTGGGAACCAAGCTGGAGTGCAGTGG |
| | | CACAATCTTGGCTCACTGCAATCTCCGCCTCCTGGGTTCAAGCGATTCTCCTGCCTCAGC |
| | | CTCCCGAGTTGTTGGGATTCCAGGCATGCATGACCAGGCTCAGCTAATTTTTGTTTTTTT |
| | | GGTAGAAACGGGGTTTCACCATATTGGCCAGGCTGGTCTCCAACTCCTAATCTCAGGTGA |
| | | TCTACCCACCTTGGCCTCCCAAATTGCTGGGATTACAGGCGTGAACCACTGCTCCCTTCC |
| | | CTGTCCTTC |
| | | (NNNNNNNNNNNNNNNNNNNNNNNNNNNNNN can be absent or can encode |
| | | a transgene of any length) |
| 989 | FAS-PTPN2- | GGATCTGCGATCGCTCCGGTGCCCGTCAGTGGGCAGAGCGCACATCGCCCACAGTCCCCG |
| | TGFBR2- | AGAAGTTGGGGGGAGGGGTCGGCAATTGAACGGGTGCCTAGAGAAGGTGGCGCGGGGTAA |
| | TGFBR2 | ACTGGGAAAGTGATGTCGTGTACTGGCTCCGCCTTTTTCCCGAGGGTGGGGGAGAACCGT |
| | quadruple | ATATAAGTGCAGTAGTCGCCGTGAACGTTCTTTTTCGCAACGGGTTTGCCGCCAGAACAC |
| | shRNA | AGCTGAAGCTTCGAGGGGCTCGCATCTCTCCTTCACGCGCCCGCCGCCCTACCTGAGGCC |
| | module, 5′ | GCCATCCACGCCGGTTGAGTCGCGTTCTGCCGCCTCCCGCCTGTGGTGCCTCCTGAACTG |
| | end | CGTCCGCCGTCTAGGTAAGTCGACTCGTTGGATCCCCACTACCCGGATCAACGCCCTAGG |
| | | TTTATGTTTGGATGAACTGACATACGCGTATCCGTCTTAAGAATCTTTTCAAACACTAGT |
| | | AGTGAAATATATATTAAACTAGTGTTTGAAAAGATTCTTATTACGGTAACGCGGAATTCG |
| | | CAACTATTTTATCAATTTTTTGCGTCGACACTTCAAGGGGCTTGCGGCCGCAACCATCTC |
| | | CATGGCTGTTTGAATGAGGCTTCAGTACTTTACAGAATCGTTGCCTGCACATCTTGGAAA |
| | | CACTTGCTGGGATTACTTCGACTTCTTAACCCAACAGAAGGCTCGAGAAGGTATATTGCT |
| | | GTTGACAGTGAGCGCCAGTGTGAAGCTCTTGTCAGATAGTGAAGCCACAGATGTATCTGA |
| | | CAAGAGCTTCACACTGATGCCTACTGCCTCGGACTTCAAGGGGCTAGAATTCGAGCAATT |
| | | ATCTTGTTTACTAAAACTGAATACCTTGCTATCTCTTTGATACATTTTTACAAAGCTGAA |
| | | TTAAAATGGTATAAATTAAATCACTTTGACGGTGACTTAGGAGTATGCCGGATCAACGCC |
| | | CTAGGTTTATGTTTGGATGAACTGACATACGCGTATCCGTCTAAAAATCAATCTCATTTC |
| | | CTGGTAGTGAAATATATATTAAACCAGGAAATGAGATTGATTTTTTTACGGTAACGCGGA |
| | | ATTCGCAACTATTTTATCAATTTTTTGCGTCGACGACTGTGACAGCAGAGTATGCCGGAT |
| | | CAACGCCCTAGGTTTATGTTTGGATGAACTGACATACGCGTATCCGTCTTATGTAAAAGA |
| | | CAAACAATGCGTAGTGAAATATATATTAAACGCATTGTTTGTCTTTTACATATTACGGTA |
| | | ACGCGGAATTCGCAACTATTTTATCAATTTTTTGCGTCGACCGGAACTATCTTGAAGAGT |
| | | AGTAGTGGACTAGTGTGACGCTGCTGACCCCTTTCTTTCCCTTCTACAGATCCAAGCTGT |
| | | GACCGGCGCCTACACCTGCAGCCCAAGCTTACC |
| 990 | FAS-PTPN2- | TAAAGGACGGGTGGCATCCCTGTGACCCCTCCCCAGTGCCTCTCCTGGCCCTGGAAGTTG |
| | TGFBR2- | CCACTCCAGTGCCCACCAGCCTTGTCCTAATAAAATTAAGTTGCATCATTTTGTCTGACT |
| | TGFBR2 | AGGTGTCCTTCTATAATATTATGGGGTGGAGGGGGGTGGTATGGAGCAAGGGGCAAGTTG |
| | quadruple | GGAAGACAACCTGTAGGGCCTGCGGGGTCTATTGGGAACCAAGCTGGAGTGCAGTGGCAC |
| | shRNA | AATCTTGGCTCACTGCAATCTCCGCCTCCTGGGTTCAAGCGATTCTCCTGCCTCAGCCTC |
| | module, 3′ | CCGAGTTGTTGGGATTCCAGGCATGCATGACCAGGCTCAGCTAATTTTTGTTTTTTTGGT |
| | end | AGAAACGGGGTTTCACCATATTGGCCAGGCTGGTCTCCAACTCCTAATCTCAGGTGATCT |
| | | ACCCACCTTGGCCTCCCAAATTGCTGGGATTACAGGCGTGAACCACTGCTCCCTTCCCTG |
| | | TCCTTC |

As used herein, “target gene” refers to a nucleic acid sequence in a cell, wherein the expression of the sequence may be specifically and effectively modulated using the nucleic acid molecules and methods described herein. In certain embodiments, the target gene may be implicated in the growth (proliferation), maintenance (survival), and/or immune behavior of an individual's immune cells. In some embodiments, the target gene is FAS. In some embodiments, the target gene is PTPN2. In some embodiments, the target gene is Transforming Growth Factor Beta Receptor 2 (TGFBR2). In some embodiments, two or more nucleic acid molecules target the TFGBR2 gene. In some embodiments, the target gene is Transforming Growth Factor Beta Receptor 1 (TGFBR1). In some embodiments, more than one target gene is modulated using a nucleic acid molecule and methods described herein. In some embodiments, at least two target genes are modulated using the nucleic acid molecules and methods described herein. In some embodiments, the nucleic acid molecule(s) is an shRNA. In some embodiments, the target genes are at least TGFBR1 and TGFBR2. In some embodiments, the target genes are at least FAS and TGFBR2. In some embodiments, the target genes are at least FAS, TGFBR1, and TGFBR2. In some embodiments, the target genes are at least FAS, TGFBR2, and PTPN2. In some embodiments, the target genes are at least FAS, PTPN2, TGFBR1, and TGFBR2.

In one aspect, provided herein are nucleic acids comprising a nucleic acid sequence at least 15 nucleotides in length complementary to a portion of the nucleic acid sequence encoding human Transforming Growth Factor Beta Receptor 2 (TGFBR2) (SEQ ID NO: 965). In some embodiments, the nucleic acid comprises a nucleic acid sequence at least 15 nucleotides in length complementary to nucleotides 2215-2236, 4430-4451, or 3761-3782 of the nucleic acid sequence encoding human Transforming Growth Factor Beta Receptor 2 (TGFBR2) set forth in SEQ ID NO: 965. In some embodiments, the nucleic acid comprises a sequence selected from the group consisting of the sequences set forth in SEQ ID NOs: 969 or 970. In some embodiments, the nucleic acid comprises the sequences set forth in SEQ ID NOs: 969 and 970.

In one aspect, provided herein are nucleic acids comprising a nucleic acid sequence at least 15 nucleotides in length complementary to a nucleic acid encoding human Transforming Growth Factor Beta Receptor 2 (TGFBR2) (SEQ ID NO: 965), wherein the nucleic acid sequence at least 15 nucleotides in length is at least 80%, 85%, 90%, 91%, 92%, 93%, 94%, 95%, 96%, 97%, 98%, 99%, or 100% complementary to a portion of the nucleic acid sequence encoding human Transforming Growth Factor Beta Receptor 2 (TGFBR2) (SEQ ID NO: 965). In some embodiments, the nucleic acid comprises at least 80%, 85%, 90%, 91%, 92%, 93%, 94%, 95%, 96%, 97%, 98%, 99%, or 100% identity to a sequence selected from the group consisting of the sequences set forth in SEQ ID NOs: 969 or 970. In some embodiments, the nucleic acid comprises at least two sequences with at least 80%, 85%, 90%, 91%, 92%, 93%, 94%, 95%, 96%, 97%, 98%, 99%, or 100% identity to a sequence selected from the group consisting of the sequences set forth in SEQ ID NOs: 969 or 970.

In some embodiments, the nucleic acid comprises a nucleic acid sequence at least 15 nucleotides in length complementary to a portion of a nucleic acid sequence encoding human Fas Cell Surface Death Receptor (FAS) set forth in SEQ ID NO: 964. In some embodiments, the nucleic acid comprises a sequence as set forth in SEQ ID NO: 967. In some embodiments, the nucleic acid sequence is complementary to nucleotides 1126 to 1364 of a nucleic acid encoding human FAS set forth in SEQ ID NO: 964. In some embodiments, the nucleic acid sequence is complementary to nucleotides 1126 to 1147 of a nucleic acid encoding human FAS set forth in SEQ ID NO: 964. In some embodiments, the nucleic acid sequence is complementary to nucleotides 1126 to 1147 of a nucleic acid encoding human FAS set forth in SEQ ID NO: 964.

In one aspect, provided herein are nucleic acid comprising a nucleic acid sequence at least 15 nucleotides in length complementary to a nucleic acid encoding human FAS set forth in SEQ ID NO: 964, wherein the nucleic acid sequence at least 15 nucleotides in length is at least 80%, 85%, 90%, 91%, 92%, 93%, 94%, 95%, 96%, 97%, 98%, 99%, or 100% complementary to a nucleic acid encoding human FAS set forth in SEQ ID NO: 964. In some embodiments, the nucleic acid comprises at least 80%, 85%, 90%, 91%, 92%, 93%, 94%, 95%, 96%, 97%, 98%, 99%, or 100% identity to a sequence as set forth in SEQ ID NO: 967.

In some embodiments, the nucleic acid comprises a nucleic acid sequence at least 15 nucleotides in length complementary to a nucleic acid encoding human Protein Tyrosine Phosphatase Non-Receptor Type 2 (PTPN2) set forth in SEQ ID NO: 966. In some embodiments, the nucleic acid comprises a sequence as set forth in SEQ ID NO: 968. In some embodiments, the nucleic acid sequence is complementary to nucleotides 518-559 of a nucleic acid encoding human PTPN2 set forth in SEQ ID NO: 966. In some embodiments, the nucleic acid sequence is complementary to nucleotides 518-539 of a nucleic acid encoding human PTPN2 set forth in SEQ ID NO: 966.

In one aspect, provided herein are nucleic acid comprising a nucleic acid sequence at least 15 nucleotides in length complementary to a nucleic acid encoding human Protein Tyrosine Phosphatase Non-Receptor Type 2 (PTPN2) set forth in SEQ ID NO: 966, wherein the nucleic acid sequence at least 15 nucleotides in length is at least 80%, 85%, 90%, 91%, 92%, 93%, 94%, 95%, 96%, 97%, 98%, 99%, or 100% complementary to a nucleic acid encoding human Protein Tyrosine Phosphatase Non-Receptor Type 2 (PTPN2) set forth in SEQ ID NO: 966. In some embodiments, the nucleic acid comprises at least 80%, 85%, 90%, 91%, 92%, 93%, 94%, 95%, 96%, 97%, 98%, 99%, or 100% identity to a sequence as set forth in SEQ ID NOs: 968.

In some embodiments, the nucleic acid is capable of reducing expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the nucleic acid is capable of reducing expression of TGFBR2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the nucleic acid is capable of reducing expression of PTPN2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the nucleic acid sequence is at least 16, 17, 18, 19, 20, 21, or 22 nucleotides in length.

In some embodiments, the nucleic acid is an RNA interference (RNAi) molecule. Exemplary RNAi molecules include short hairpin RNA (shRNA), a small interfering RNA (siRNA), a double stranded RNA (dsRNA), or an antisense oligonucleotide. In some embodiments, the nucleic acid is a short hairpin RNA (shRNA), a small interfering RNA (siRNA), a double stranded RNA (dsRNA), or an antisense oligonucleotide. In some embodiments, the nucleic acid is an shRNA.

Single-stranded hairpin ribonucleic acids (shRNAs) are short duplexes where the sense and antisense strands are linked by a hairpin loop. They consist of a stem-loop structure that can be transcribed in cells from an RNA polymerase II or RNA polymerase III promoter on a plasmid construct. Once expressed, shRNAs are processed into RNAi species. Expression of shRNA from a plasmid is known to be relatively stable, thereby providing strong advantages over, for example, the use of synthetic siRNAs. shRNA expression units may be incorporated into a variety of plasmids, liposomes, viral vectors, and other vehicles for delivery and integration into a target cell. Expression of shRNA from a plasmid can be stably integrated for constitutive expression. shRNAs are synthesized in the nucleus of cells, further processed and transported to the cytoplasm, and then incorporated into the RNA-induced silencing complex (RISC) for activity. The shRNAs are converted into active siRNA molecules (which are capable of binding to, sequestering, and/or preventing the translation of mRNA transcripts encoded by target genes).

The Argonaute family of proteins is the major component of RISC. Within the Argonaute family of proteins, only Ago2 contains endonuclease activity that is capable of cleaving and releasing the passenger strand from the stem portion of the shRNA molecule. The remaining three members of Argonaute family, Ago1, Ago3 and Ago4, which do not have identifiable endonuclease activity, are also assembled into RISC and are believed to function through a cleavage-independent manner. Thus, RISC can be characterized as having cleavage-dependent and cleavage-independent pathways.

RNAi (e.g., antisense RNA, siRNA, microRNA, shRNA, etc.) are described in International Publication Nos. WO2018232356A1, WO2019084552A1, WO2019226998A1, WO2020014235A1, WO2020123871A1, and WO2020186219A1, each of which is herein incorporated by reference for all purposes.

Antisense oligonucleotide structure and chemical modifications are described in International PCT Publication No. WO20/132521, which is hereby incorporated by reference.

dsRNA and shRNA molecules and methods of use and production are described in U.S. Pat. Nos. 8,829,264; 9,556,431; and 8,252,526, each of which are hereby incorporated by reference

siRNA molecules and methods of use and production are described in U.S. Pat. No. 7,361,752 and US Patent Application No. US20050048647, both of which are hereby incorporated by reference.

Additional methods and compositions for RNA interference such as shRNA, siRNA, dsRNA, and antisense oligonucleotides are generally known in the art, and are further described in U.S. Pat. Nos. 7,361,752; 8,829,264; 9,556,431; 8,252,526, International PCT Publication No. WO00/44895; International PCT Publication No. WO01/36646; International PCT Publication No. WO99/32619; International PCT Publication No. WO00/01846; International PCT Publication No. WO01/29058; and International PCT Publication No. WO00/44914; International PCT Publication No. WO04/030634; International PCT Publication No. WO 2024059618; each of which are hereby incorporated by reference.

The nucleic acid sequences (or constructs) that may be used to encode the RNAi molecules, such as an shRNA described herein, may comprise a promoter, which is operably linked (or connected), directly or indirectly, to a sequence encoding the RNAi molecules. Such promoters may be selected based on the host cell and the effect sought. Non-limiting examples of suitable promoters include constitutive and inducible promoters, such as EF1α or inducible Hepatocyte Nuclear Factor 1α (HNF1α)-YB TATA or RNA polymerase II (pol II)-based promoters. In some embodiments, the constitutive promoter is EF1α. In some embodiments, the EF1α promoter comprises as sequence as set forth in SEQ ID NO: 991. Non-limiting examples of suitable promoters further include the tetracycline inducible or repressible promoter, RNA polymerase I or III-based promoters, the pol II dependent viral promoters, such as the CMV-IE promoter, and the pol III U6 and H1 promoters, as well as Hepatocyte Nuclear Factor 1α (HNF1α)-YB TATA promotor provided in SEQ ID NO: 992. The bacteriophage T7 promoter may also be used (in which case it will be appreciated that the T7 polymerase must also be present). The nucleic acid sequences need not be restricted to the use of any single promoter, especially since the nucleic acid sequences may comprise two or more shRNAs (i.e., a combination of effectors), including but not limited to incorporated shRNA molecules. Each incorporated promoter may control one, or any combination of, the shRNA molecule components.

In certain embodiments, the promoter may be preferentially active in the targeted cells, e.g., it may be desirable to preferentially express at least one nucleic acid in immune cells using an immune cell-specific promoter. Introduction of such constructs into host cells may be effected under conditions whereby the two or more nucleic acids that are contained within the nucleic acid precursor transcript initially reside within a single primary transcript, such that the separate RNA molecules (for example, shRNA each comprising its own stem-loop structure) are subsequently excised from such precursor transcript by an endogenous ribonuclease. The resulting mature nucleic acids (e.g., shRNAs) may then induce degradation, and/or translation repression, of target gene nucleic acid (e.g., mRNA) transcripts produced in the cell. Alternatively, each of the precursor stem-loop structures may be produced as part of a separate transcript, in which case each nucleic acid sequence will preferably include its own promoter and transcription terminator sequences. Additionally, the multiple nucleic acid precursor transcripts may reside within a single primary transcript.

The stem-loop structures of the shRNA nucleic acids described herein may be about 40 to 100 nucleotides long or, preferably, about 50 to 75 nucleotides long. The stem region may be about 15-45 nucleotides in length (or more), or about 20-30 nucleotides in length. In some embodiments, the stem region is 22 nucleotides in length. In some embodiments, the stem region is 15, 16, 17, 18, 19, 20, 21, 22, 23, 24, 25, 26, 27 28 29, 30, 31, 32, 33, 34, 35, 36, 37, 38, 39, 40, 41, 42, 43, 44, or 45 nucleotides in length.

The stem may comprise a perfectly complementary duplex (but for any 3′ tail), however, bulges or interior loops may be present on either arm of the stem. The number of such bulges and asymmetric interior loops are preferably few in number (e.g., 1, 2 or 3) and are about 3 nucleotides or less in size. The terminal loop portion may comprise about 4 or more nucleotides, but preferably not more than about 25. The loop portion will preferably be 6-15 nucleotides in size.

As described herein, the stem regions of the shRNAs comprise passenger strands and guide strands, whereby the guide strands contain sequences complementary to the target nucleic acid (e.g., mRNA) transcript encoded by the target gene(s). Preferably, the G-C content and matching of guide strand and passenger strand is carefully designed for thermodynamically-favorable strand unwind activity with or without endonuclease cleavage. Furthermore, the specificity of the guide strand is preferably confirmed via a BLAST search (www.ncbi.nim.nih.gov/BLAST).

The disclosure herein provides that the expression level of multiple target genes may be modulated using the methods and nucleic acids described herein. For example, the disclosure herein provides that a first set of nucleic acids may be designed to include a sequence (a guide strand) that is designed to reduce the expression level of a first target gene, whereas a second set of nucleic acids may be designed to include a sequence (a guide strand) that is designed to reduce the expression level of a second target gene. The different sets of nucleic acids may be expressed and reside within the same, or separate, preliminary transcripts. In certain embodiments, such multiplex approach, i.e., the use of the nucleic acids described herein to modulate the expression level of two or more target genes, may have an enhanced therapeutic effect on a patient. For example, if a patient is provided with cells expressing the nucleic acid molecules described herein to treat, prevent, or ameliorate the effects of cancer, it may be desirable to provide the patient with two or more types of nucleic acid molecules, which are designed to reduce the expression level of multiple genes that are implicated in activation or repression of immune cells.

The nucleic acid molecule(s) described herein may be capable of reducing target gene expression in a cell by at least more than about 50% as compared to a control cell that does not comprise the nucleic acid molecule(s). For example, the nucleic acid molecule(s) (e.g., shRNA) can be capable of reducing expression of a target gene selected from the group consisting of FAS and TGBFR2 in the immune cell by at least 10%, 15%, 20%, 25%, 30%, 35%, 40%, 45%, 50%, 55%, 60%, 65%, 75%, 80%, 85%, 90%, 95%, 97%, 98%, 99%, or more as compared to a control cell that does not comprise the nucleic acid molecule(s). The nucleic acid molecule(s) can be capable of reducing expression of a target gene selected from the group consisting of FAS and TGBFR2 in the immune cell by at least between about 50-100%, 50-99%, 50-95%, 50-90%, 50-85%, 50-80%, 50-75%, 50-70%, 50-65%, 50-60%, 50-55%, or as compared to a control cell that does not comprise the nucleic acid molecule(s). In some embodiments, the nucleic acid molecule(s) is capable of reducing expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid molecule(s). In some embodiments, the nucleic acid molecule(s) is capable of reducing expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid molecule(s). In some embodiments, the nucleic acid molecule(s) is capable of reducing expression of TGBFR2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid molecule(s).

The nucleic acid molecule(s) may be chemically synthesized, or in vitro transcribed, and may further include one or more modifications to phosphate-sugar backbone or nucleosides residues.

Other methods known in the art for introducing nucleic acids to cells may be used, such as lipid-mediated carrier transport, chemical mediated transport, such as calcium phosphate, and the like. Thus, the nucleic acid molecule(s) construct may be introduced along with components that perform one or more of the following activities: enhance RNA uptake by the cell, promote annealing of the duplex strands for shRNA, stabilize the annealed shRNA strands, or otherwise increase inhibition of the target gene.

### Nucleic Acids and Vectors

In various embodiments, the present disclosure contemplates nucleic acid inserts that comprise one or more transgenes encoding the priming receptors, CARs, or suppressors of gene expression as described herein. In some embodiments, the nucleic acids are recombinant nucleic acids. In some embodiments, the nucleic acids are synthetic nucleic acids. In some embodiments, the insert encodes a priming receptor transgene. In some embodiments, the insert encodes a CAR transgene. In some embodiments, the insert comprises one or more suppressors of gene expression. In some embodiments, the insert comprises a priming receptor transgene and a CAR transgene. In some embodiments, the insert comprises a priming receptor transgene and one or more suppressors of gene expression. In some embodiments, the insert comprises a CAR transgene and one or more suppressors of gene expression. In some embodiments, the insert comprises a CAR transgene, a priming receptor transgene, and a suppressor of gene expression.

In one aspect, provided herein are nucleic acids comprising a nucleotide sequence that is at least 95%, 96%, 97%, 98%, 99%, or 100% identical to SEQ ID NO: 1242, which contains functional domains and wherein the activity of the functional domains is not altered relative to those in a nucleic acid comprising or consisting of SEQ ID NO: 1242. For example, the nucleotide differences can be silent substitutions, additions or deletions of nucleotides.

In one aspect, provided herein are nucleic acids comprising a nucleotide sequence that is at least 95%, 96%, 97%, 98%, 99%, or 100% identical to SEQ ID NO: 1124, which contains functional domains and wherein the activity of the functional domains is not altered relative to those in a nucleic acid comprising or consisting of SEQ ID NO: 1124. For example, the nucleotide differences can be silent substitutions, additions or deletions of nucleotides.

In one aspect, provided herein is a nucleic acid comprising SEQ ID NO: 1242, or a nucleic acid at least 95%, 96%, 97%, 98%, 99%, or 100% identical to SEQ ID NO: 1242.

In one aspect, provided herein is a nucleic acid comprising SEQ ID NO: 1124, or a nucleic acid at least 95%, 96%, 97%, 98%, 99%, or 100% identical to SEQ ID NO: 1124.

In some embodiments, the nucleic acid is a linear nucleic acid. In some embodiments, the nucleic acid is a circular nucleic acid. In some embodiments, the nucleic acid further comprises an additional 5′ and/or 3′ nucleotide sequence(s). In some embodiments, the additional 5′ and/or 3′ nucleotide sequence(s) comprises from 1-100 nucleotides, optionally 1, 5, 10, 20, 30, 40, 50, 60, 70, 80, 90 or 100 nucleotides or between 1-10, 10-20, 20-30, 30-40, 40-50, 50-60, 60-70, 70-80, 80-90, or 90-100 nucleotides. In some embodiments, the additional 5′ nucleotide sequence and the additional 3′ nucleotide sequence each comprise a protelomerase binding sequence.

In some embodiments, the nucleic acid is a closed end DNA (ceDNA).

### Additional Elements

In some embodiments, the one or more nucleic acid(s) further comprises a 5′ homology directed repair arm and/or a 3′ homology directed repair arm complementary to an insertion site in a host cell chromosome. In some embodiments, the one or more nucleic acid(s) comprises the 5′ homology directed repair arm and the 3′ homology directed repair arm. In some embodiments, the one or more nucleic acid(s) is incorporated into an expression cassette or an expression vector. In some embodiments, the expression cassette or the expression vector further comprises a constitutive promoter upstream of the one or more nucleic acid(s).

For example, in the exemplary Logic Gates provided herein, the nucleotide sequences comprising a 5′ homology directed repair arm and a 3′ homology directed repair arm complementary to the GS94 locus insertion site comprise nucleotides 24-473 and 7258-7707 of SEQ ID NO: 1120; nucleotides 24-473 and 7240-7689 of SEQ ID NO: 1121; nucleotides 24-473 and 7622-8071 of SEQ ID NO: 1122; nucleotides 24-473 and 7637-8086 of SEQ ID NO: 1123; or nucleotides 24-473 and 7622-8071 of SEQ ID NO: 1124. In some embodiments, the vector provided herein comprises nucleotides 24-473 and 7258-7707 of SEQ ID NO: 1120; nucleotides 24-473 and 7240-7689 of SEQ ID NO: 1121; nucleotides 24-473 and 7622-8071 of SEQ ID NO: 1122; nucleotides 24-473 and 7637-8086 of SEQ ID NO: 1123; or nucleotides 24-473 and 7622-8071 of SEQ ID NO: 1124.

In some embodiments, the nucleotide sequences that are homologous to genomic sequences flanking the GS94 locus insertion site comprise SEQ ID NOs: 1235 and 1236. In some embodiments, the vector comprises homology regions to the gRNA of the RNP complex used for inserting the nucleic acid into the genome of a cell. In some embodiments, the sequences of the gRNA homology regions comprise SEQ ID NOs: 932 and 1237.

In some embodiments, the priming receptor, CAR, first nucleic acid, and the second nucleic acid are incorporated into a single expression cassette or a single expression vector. In some embodiments, the priming receptor, CAR, first nucleic acid, and the second nucleic acid are incorporated into two or more expression cassettes or expression vectors. In some embodiments, the expression vector(s) is a non-viral vector.

The one or more interfering nucleic acid sequences (e.g., one or more shRNA) can be encoded in the intron regions of the recombinant nucleic acid insert, DNA template, single expression cassette, or a single expression vector that also encodes the priming receptor and/or the CAR. For example, if the DNA template includes promoters, such as EF1α, or inducible promoters such as the HNF1α-YB TATA promoter, described herein, to drive expression of the CAR or priming receptor, the one or more nucleic acid sequences (e.g., shRNA sequences) can be encoded in the promoter intronic region. In some embodiments, the one or more nucleic acid sequences is encoded in at least one intron region of the nucleic acid insert, module, cassette, or DNA template. In some embodiments, the one or more nucleic acid sequences is encoded in at least one EF1α intron region of the nucleic acid insert, module, cassette, or DNA template.

In some embodiments, the present disclosure contemplates nucleic acid(s), modules, cassettes, or DNA template inserts that comprise one or more transgenes encoding the priming receptors and/or CARs as described herein. In some embodiments, the DNA template insert or cassette encodes a priming receptor transgene. In some embodiments, the DNA template insert or cassette encodes a chimeric antigen receptor transgene. In some embodiments, the DNA template insert encodes a first nucleic acid complementary to at least 15 nucleotides of a human FAS nucleic acid sequence, and a second nucleic acid complementary to at least 15 nucleotides of a human PTPN2 or TGFBR2 nucleic acid sequence. In some embodiments, the DNA template insert comprises a priming receptor transgene and a chimeric antigen receptor transgene. In some embodiments, the DNA template insert comprises a priming receptor transgene, a chimeric antigen receptor transgene, a first nucleic acid complementary to at least 15 nucleotides of a human FAS nucleic acid A sequence, and a second nucleic acid complementary to at least 15 nucleotides of a human PTPN2 or TGFBR2 nucleic acid sequence. In some embodiments, the DNA template insert comprises a priming receptor transgene, a chimeric antigen receptor transgene, a first nucleic acid complementary to at least 15 nucleotides of a human FAS nucleic acid sequence, and a second nucleic acid complementary to at least 15 nucleotides of a human PTPN2 nucleic acid sequence.

In some embodiments, the one or more recombinant nucleic acid(s) are encoded on a single DNA template insert. In some embodiments, the one or more recombinant nucleic acid(s) are encoded on multiple DNA template inserts. For example, the one or more recombinant nucleic acid(s) can be encoded on two, three, or four DNA template inserts.

The DNA template insert can also comprise a self-cleaving peptide. Examples of self-cleaving peptides include, but are not limited to, self-cleaving viral 2A peptides, for example, a porcine teschovirus-1 (P2A) peptide, a Thosea asigna virus (T2A) peptide, an equine rhinitis A virus (E2A) peptide, or a foot-and-mouth disease virus (F2A) peptide. Self-cleaving 2A peptides allow expression of multiple gene products from a single construct. (See, for example, Chang et al. “Cleavage efficient 2A peptides for high level monoclonal antibody expression in CHO cells,” MAbs 7(2): 403-412 (2015)).

The DNA template insert can also comprise a WPRE element. WPRE elements are generally described in Higashimoto, T., et al. Gene Ther 14, 1298-1304 (2007); and Zufferey, R., et al. J Virol. 1999 April; 73(4):2886-92., both of which are hereby incorporated by reference. An exemplary WPRE element is also provided in SEQ ID NO: 1243.

The DNA template insert can also comprise a synthetic polyA signal, an SV40 polyA signal, a human growth hormone (GH1) polyA signal, or a bovine growth hormone (bGH) polyA signal. In some embodiments, the polyA signal comprises the sequence as set forth in SEQ ID NOs: 993, 994, 995, or 1244.

Table 21 provides the sequences of exemplary polyadenylation (polyA) signal sequences.

TABLE 21
Exemplary polyadenylation (polyA) signal sequences

| SEQ | | |
|---|---|---|
| ID | | |
| NO | Name | Sequence |
| 993 | 2X Synthetic | aataaaagatctttaatgaaaatagatctgtgtgttggttttttgtgtg |
| | polyA | aataaaagatccagagctctagagatctgtgtgttggttttttgtgtg |
| 994 | Human GH1 | cgggtggcatccctgtgacccctccccagtgcctctcctggccctggaa |
| | polyA | gttgccactccagtgcccaccagccttgtcctaataaaattaagttgca |
| | | tcattttgtctgactaggtgtccttctataatattatggggtggagggg |
| | | ggggtatggagcaaggggcaagttgggaagacaacctgtagggcctgcg |
| | | gggtctattgggaaccaagctggagtgcagtggcacaatcttggctcac |
| | | tgcaatctccgcctcctgggttcaagcgattctcctgcctcagcctccc |
| | | gagttgttgggattccaggcatgcatgaccaggctcagctaatttttgt |
| | | ttttttggtagaaacggggtttcaccatattggccaggctggtctccaa |
| | | ctcctaatctcaggtgatctacccaccttggcctcccaaattgctggga |
| | | ttacaggcgtgaaccactgctcccttccctgtccttc |
| 995 | Bovine GH | cgactgtgccttctagttgccagccatctgttgtttgcccctcccccgt |
| | polyA | gccttccttgaccctggaaggtgccactcccactgtcctttcctaataa |
| | | aatgaggaaattgcatcgcattgtctgagtaggtgtcattctattctgg |
| | | ggggggggtggggcaggacagcaagggggaggattgggaagacaatagc |
| | | aggcatgctggggatgcggtgggctctatgg |
| 1244 | SV40 polyA | aacttgtttattgcagcttataatggttacaaataaagcaatagcatca |
| | | caaatttcacaaataaagcatttttttcactgcattctagttgtggttt |
| | | gtccaaactcatcaatgtatcttatcatgtctgg |

### Cells

Also provided herein are cells (e.g., immune cells) comprising at least one DNA template non-virally inserted into a target region of the genome of the cell, wherein DNA template encodes the priming receptor and CAR system as described herein, optionally also the gene expression suppressor molecule. Also provided herein are immune cells comprising a priming receptor that specifically binds SLC43A2 and a chimeric antigen receptor that specifically binds TMPRSS4. The cell can further comprise a gene expression suppressor such as an RNAi molecule (e.g., shRNA) or an sgRNA for CRISPR-based knockout of a target gene.

A cell, such as a human cell, comprising a DNA template insert at a target locus or safe harbor site as described in the present disclosure can be referred to as an engineered cell, e.g. an engineered human cell, or a recombinant cell, e.g., a recombinant human cell. In some embodiments, the immune cell is any cell that can give rise to a pluripotent immune cell. In some embodiments, the immune cell is a primary immune cell. In some embodiments, the immune cell can be an induced pluripotent stem cell (iPSC) or a human pluripotent stem cell (HSPC). In some embodiments, the immune cell comprises primary hematopoietic cells or primary hematopoietic stem cells. In some embodiments, that engineered cell is a stem cell, a human cell, a primary cell, an hematopoietic cell, an adaptive immune cell, an innate immune cell, a natural killer (NK) cell, a T cell, a CD8+ cell, a CD4+ cell, or a T cell progenitor. In some embodiments, the immune cells are T cells. In some embodiments, the T cells are regulatory T cells, effector T cells, or naïve T cells. In some embodiments, the T cells are CD8⁺ T cells. In some embodiments, the T cells are CD4⁺ T cells. In some embodiments, the T cells are CD4⁺CD8⁺ T cells.

In some embodiments, the engineered cell is a stem cell, a human cell, a primary cell, an hematopoietic cell, an hematopoietic stem cell, an adaptive immune cell, an innate immune cell, a T cell or a T cell progenitor. Non-limiting examples of immune cells that are contemplated in the present disclosure include T cell, B cell, natural killer (NK) cell, NKT/iNKT cell, macrophage, myeloid cell, and dendritic cells. Non-limiting examples of stem cells that are contemplated in the present disclosure include pluripotent stem cells (PSCs), embryonic stem cells (ESCs), induced pluripotent stem cells (iPSCs), embryo-derived embryonic stem cells obtained by nuclear transfer (ntES; nuclear transfer ES), male germline stem cells (GS cells), embryonic germ cells (EG cells), hematopoietic stem/progenitor stem cells (HSPCs), somatic stem cells (adult stem cells), hemangioblasts, neural stem cells, mesenchymal stem cells and stem cells of other cells (including osteocyte, chondrocyte, myocyte, cardiac myocyte, neuron, tendon cell, adipocyte, pancreocyte, hepatocyte, nephrocyte and follicle cells and so on). In some embodiments, the engineered cells is a T cell, NK cells, iPSC, and HSPC. In some embodiments, the engineered cells used in the present disclosure are human cell lines grown in vitro (e.g., deliberately immortalized cell lines, cancer cell lines, etc.). In some embodiments, the engineered cells are autologous. In some embodiments, the engineered cells are allogeneic.

In one aspect, provided herein are cells comprising a nucleotide sequence comprising SEQ ID NO: 1242 or a nucleotide sequence that differs therefrom in at most 50 nucleotides, wherein the differences are silent substitutions, additions or deletions.

In one aspect, provided herein are cells comprising a nucleotide sequence comprising SEQ ID NO: 1124 or a nucleotide sequence that differs therefrom in at most 50 nucleotides, wherein the differences are silent substitutions, additions or deletions.

In some embodiments, the cell is a human cell. In some embodiments, the cell is a primary cell. In some embodiments, the cell is a T cell. In some embodiments, the cell is manufactured from a cell obtained from a human subject.

In some embodiments, the cell comprises at least one protein encoded by SEQ ID NO: 1124 or SEQ ID NO: 1242.

Also provided herein are populations of cells comprising a plurality of the immune cell. In some embodiments, the genome of at least 20%, 30%, 40%, 50%, 60%, 70%, 80%, 90%, 95%, 99% or greater of the cells comprises the priming receptor and CAR system and/or gene suppressor as described herein.

### Methods of Treating Immune-Related Conditions or Diseases

In one aspect, the disclosure provides methods of treating an immune-related condition (e.g., cancer) in a subject comprising administering to the subject an effective amount of a composition, e.g., a cell or population of cells, comprising a synthetic immune receptor that specifically binds to TMPRSS4 and/or SLC34A2. In one aspect, the invention provides methods of treating an immune-related condition (e.g., cancer) in a subject comprising administering to the subject an effective amount of a cell or population of cells comprising a system comprising a synthetic transcriptional modulator (e.g., a priming receptor) that specifically binds to human SLC34A2 and a synthetic immune receptor (e.g., a CAR) that specifically binds to human TMPRSS4. In some embodiments, the composition further comprises a first nucleic acid sequence at least 15 nucleotides in length, wherein the first nucleic acid sequence is complementary to a portion of the nucleic acid sequence encoding human Fas Cell Surface Death Receptor (FAS) set forth in SEQ ID NO: 964, and at least one second nucleic acid sequence at least 15 nucleotides in length, wherein the second nucleic acid sequence is complementary to a portion of the a nucleic acid sequence encoding human TGFBR2 set forth in SEQ ID NO: 965. In some embodiments, the synthetic immune receptor that specifically binds to TMPRSS4 is a chimeric antigen receptor that specifically binds to TMPRSS4. In some embodiments, the synthetic immune receptor that specifically binds to SLC34A2 is a priming receptor that specifically binds to SLC34A2.

In one aspect, the disclosure provides methods of enhancing an immune response, e.g., for killing cancer cells, in a subject comprising administering to the subject an effective amount of a composition, e.g., a cell or population of cells, comprising a synthetic immune receptor that specifically binds to human TMPRSS4 and/or human SLC34A2. In one aspect, the disclosure provides methods of enhancing an immune response, e.g., for killing cancer cells, in an individual comprising administering to the subject an effective amount of a composition comprising a system comprising a priming receptor that specifically binds to SLC34A2, a synthetic immune receptor that specifically binds to TMPRSS4, a first nucleic acid sequence at least 15 nucleotides in length, wherein the first nucleic acid sequence is complementary to a nucleic acid encoding human Fas Cell Surface Death Receptor (FAS) set forth in SEQ ID NO: 964, and a second nucleic acid sequence at least 15 nucleotides in length, wherein the second nucleic acid sequence is complementary to a nucleic acid encoding human Phosphatase Non-Receptor Type 2 (PTPN2) set forth in SEQ ID NO: 966; or complementary to a nucleic acid sequence encoding human Transforming Growth factor (TGF)-β Receptor 2 (TGFBR2) set forth in SEQ ID NO: 965.

In one aspect, the disclosure provides methods of inhibiting (e.g., killing, disabling, causing cytolysis of, or preventing growth or expansion) of a target cell or target tissue that expresses both TMPRSS4 and SLC34A2. In one aspect, the invention provides methods of killing or causing cytolysis of, a target cell or target tissue that expressed both TMPRSS4 and SLC34A2. In some embodiments, the target cell is a cancer cell or the target tissue is a cancer tissue.

In one aspect, the invention provides methods of inducing cytolysis of a target cell in a subject comprising administering to the subject an effective amount of a composition (such as a cell or a composition of cells (e.g., a population of cells)) comprising a synthetic immune receptor that specifically binds to TMPRSS4 and/or SLC34A2. In one aspect, the invention provides methods of enhancing an immune response in a subject comprising administering to the subject an effective amount of a composition (such as a cell or a composition of cells (e.g., a population of cells) comprising a synthetic immune receptor that specifically binds to TMPRSS4 and/or SLC34A2. In one aspect, the invention provides methods of enhancing an immune response in a subject comprising administering to the subject an effective amount of a composition (such as a cell or a composition of cells (e.g., a population of cells)) comprising a system comprising a priming receptor that specifically binds to SLC34A2, a synthetic immune receptor that specifically binds to TMPRSS4, a first nucleic acid sequence at least 15 nucleotides in length, wherein the first nucleic acid sequence is complementary to a nucleic acid encoding human Fas Cell Surface Death Receptor (FAS) comprising the sequence set forth in SEQ ID NO: 964, and a second nucleic acid sequence at least 15 nucleotides in length, wherein the second nucleic acid sequence is complementary to a nucleic acid encoding human Phosphatase Non-Receptor Type 2 (PTPN2) comprising the sequence set forth in SEQ ID NO: 966; or complementary to a nucleic encoding human Transforming Growth factor (TGF)-β Receptor 2 (TGFBR2) comprising the sequence set forth in SEQ ID NO: 965.

In some embodiments, the nucleic acid is an shRNA molecule. In some embodiments, the shRNA is selected from the group consisting of a FAS shRNA molecule, a PTPN2 shRNA molecule, and a TGFBR2 shRNA molecule. In some embodiments, the cell comprises at least a FAS shRNA molecule. In some embodiments, the cell comprises at least a PTPN2 shRNA molecule. In some embodiments, the cell comprises at least a TGFBR2 shRNA molecule. In some embodiments, the cell comprises at least a second TGFBR2 shRNA molecule. In some embodiments, the cell comprises at least a FAS shRNA molecule and a PTPN2 shRNA molecule. In some embodiments, the cell comprises at least a FAS shRNA molecule and a TGFBR2 shRNA molecule. In some embodiments, the cell comprises at least a PTPN2 shRNA molecule and a TGFBR2 shRNA molecule. In one aspect, the invention provides methods of enhancing an immune response in a subject comprising administering to the subject an effective amount of a composition comprising a cell comprising at least one shRNA molecule, wherein the shRNA molecule is selected from the group consisting of a FAS shRNA molecule, a PTPN2 shRNA molecule, and a TGFBR2 shRNA molecule.

In some embodiments, the methods provided herein are useful for the treatment of an immune-related condition in a subject. In some embodiments, the immune-related condition is cancer. In one embodiment, the subject is a human.

In some embodiments, the methods provided herein (such as methods of enhancing an immune response or inducing cytolysis of a target cell) are useful for the treatment of cancer and as such a subject receiving the system described herein has cancer. In some embodiments, the cancer is a solid cancer. In some embodiments, the cancer is a liquid cancer. In some embodiments, the cancer is immunoevasive. In some embodiments, the cancer is immunoresponsive. In particular embodiments, the cancer is non-small cell lung cancer (NSCLC), ovarian cancer, cervical cancer, endometrial cancer, uterine cancer, pancreatic cancer, esophageal cancer, head and neck squamous cell cancer, thyroid cancer, bladder cancer, breast cancer, cholangiocarcinoma cancer, colon cancer, rectal cancer, kidney cancer, renal cell carcinoma, prostate cancer, stomach cancer, or gastric cancer. In some embodiments, the cancer or cancer cell expresses both TMPRSS4 and SLC34A2.

In some embodiments, the treatment results in a decrease in the cancer volume or size. In some embodiments, the treatment is effective at reducing a cancer volume as compared to the cancer volume prior to administration of the antibody. In some embodiments, the treatment results in a decrease in the cancer growth rate. In some embodiments, the treatment is effective at reducing a cancer growth rate as compared to the cancer growth rate prior to administration of the antibody. In some embodiments, the treatment is effective at eliminating the cancer. In some embodiments, the treatment is effective at killing the cancer or cancer cells.

In some embodiments, TMPRSS4 and/or SLC34A2 are expressed at a higher level in the target cell as compared to a non-target cell. In some embodiments, TMPRSS4 and/or SLC34A2 are expressed at a higher level in the cancer cell as compared to a non-cancer cell. Levels of TMPRSS4 and/or SLC34A2 RNA or protein expression can be assessed by any technique known in the field, including, but not limited to, protein assays or nucleic assays such as FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometry, HPLC, qPCR, RT-qPCR, multiplex qPCR or RT-qPCR, RNA-seq, microarray analysis, SAGE, MassARRAY technique, and FISH, and combinations thereof.

### Methods of Immune Modulation and Induced Cytolysis

Methods of administration of a cell comprising a synthetic immune receptor that specifically binds to TMPRSS4 and/or SLC34A2 or a cell comprising a system comprising a priming receptor that specifically binds to SLC34A2 and a chimeric antigen receptor that specifically binds to TMPRSS4 to modulate an immune response are provided herein. Modulation can be an increase or decrease in an immune response. In some embodiments, modulation is an increase in an immune response. In some embodiments, the immune response is secretion of pro-inflammatory cytokines or chemokines, or T cell-mediated cytotoxicity. In some embodiments, the immune response is inducing a cytolytic response (e.g., T cell-mediated cytotoxicity) in a target cell by a cell expressing the system. In some embodiments, the immune response is killing a target cell by a cell expressing the system.

In one aspect, provided herein are methods of inducing cytolysis (e.g., via T cell-mediated cytotoxicity) by or killing a target cell by contacting the target cell with a cell comprising a system comprising a priming receptor that specifically binds to SLC34A2 and a chimeric antigen receptor that specifically binds to TMPRSS4, wherein the target cell expresses SLC34A2 and TMPRSS4. In some embodiments, the target cell is a cancer cell. In some embodiments, the contacting happens in vivo in a subject.

In one aspect, administration of a cell comprising a synthetic immune receptor that specifically binds to TMPRSS4 and/or SLC34A2 or a cell comprising a system comprising a priming receptor that specifically binds to SLC34A2 and a chimeric antigen receptor that specifically binds to TMPRSS4 as described herein can result in induction of pro-inflammatory molecules, such as cytokines or chemokines. Generally, induced pro-inflammatory molecules are present at levels greater than that achieved with isotype control. Such pro-inflammatory molecules in turn result in activation of anti-tumor immunity, including, but not limited to, T cell activation, T cell proliferation, T cell differentiation, M1-like macrophage activation, and NK cell activation. Thus, the administration of a cell comprising a synthetic immune receptor that specifically binds to TMPRSS4 and/or SLC34A2 or a cell comprising a system comprising a priming receptor that specifically binds to SLC34A2 and a chimeric antigen receptor that specifically binds to TMPRSS4 can induce multiple anti-tumor immune mechanisms that lead to tumor destruction or cytolysis of tumor cells.

In one aspect, administration of a cell comprising a synthetic immune receptor that specifically binds to TMPRSS4 and/or SLC34A2 or a cell comprising a system comprising a priming receptor that specifically binds to SLC34A2 and a chimeric antigen receptor that specifically binds to TMPRSS4 as described herein can result in T cell-mediated cytotoxicity. Generally, cytotoxic T cells kill target cells bearing specific antigen(s), (e.g., such as SLC34A2 and TMPRSS4) and do not kill neighboring cells that do not express the specific antigen(s).

In one aspect, provided herein are methods of increasing an immune response (e.g., inducing a pro-inflammatory response or T cell-mediated cytotoxicity) in a subject comprising administering to the subject an effective amount of a cell comprising a synthetic immune receptor that specifically binds to TMPRSS4 and/or SLC34A2 or a cell comprising a system comprising a priming receptor that specifically binds to SLC34A2 and a chimeric antigen receptor that specifically binds to TMPRSS4. In some embodiments, the method of increasing an immune response in a subject comprises administering to the subject a cell comprising a synthetic immune receptor that specifically binds to TMPRSS4 and/or SLC34A2 or a cell comprising a system comprising a priming receptor that specifically binds to SLC34A2 and a chimeric antigen receptor that specifically binds to TMPRSS4.

In some embodiments, the cell is present in a pharmaceutical composition further comprising a pharmaceutically acceptable excipient.

In any and all aspects of increasing an immune response as described herein, any increase or decrease or alteration of an aspect of characteristic(s) or function(s) is as compared to a cell not comprising a composition comprising a synthetic immune receptor that specifically binds to TMPRSS4 and/or SLC34A2 or a system comprising a priming receptor that specifically binds to SLC34A2 and a chimeric antigen receptor that specifically binds to TMPRSS4.

Increasing an immune response can be both enhancing an immune response or inducing an immune response. For instance, increasing an immune response encompasses both the start or initiation of an immune response, or ramping up or amplifying an on-going or existing immune response. In some embodiments, the treatment induces an immune response. In some embodiments, the induced immune response is an adaptive immune response. In some embodiments, the induced immune response is an innate immune response. In some embodiments, the treatment enhances an immune response. In some embodiments, the enhanced immune response is an adaptive immune response. In some embodiments, the enhanced immune response is an innate immune response. In some embodiments, the treatment increases an immune response. In some embodiments, the increased immune response is an adaptive immune response. In some embodiments, the increased immune response is an innate immune response. In some embodiments, the immune response is started or initiated by administration of a cell comprising a synthetic immune receptor that specifically binds to TMPRSS4 or a cell comprising a system comprising a priming receptor that specifically binds to SLC34A2 and a chimeric antigen receptor that specifically binds to TMPRSS4. In some embodiments, the immune response is enhanced by administration of a cell comprising a synthetic immune receptor that specifically binds to TMPRSS4 or a cell comprising a system comprising a priming receptor that specifically binds to SLC34A2 and a chimeric antigen receptor that specifically binds to TMPRSS4.

In one aspect, the present application provides methods of genetically editing a cell with a synthetic immune receptor that specifically binds to TMPRSS4 and/or SLC34A2 or a system comprising a priming receptor that specifically binds to SLC34A2 and a synthetic immune receptor that specifically binds to TMPRSS4. In some embodiments, the cell is further genetically edited to comprise a first nucleic acid sequence at least 15 nucleotides in length complementary to a nucleic acid encoding human Fas Cell Surface Death Receptor (FAS) comprising the sequence set forth in SEQ ID NO: 964, and a second nucleic acid sequence at least 15 nucleotides in length complementary to a nucleic acid encoding human Phosphatase Non-Receptor Type 2 (PTPN2) comprising the sequence set forth in SEQ ID NO: 966; or complementary to a nucleic acid encoding human Transforming Growth factor (TGF)-β Receptor 2 (TGFBR2) comprising the sequence set forth in SEQ ID NO: 965, which results in the modulation of the immune function of the cell. The modulation can be increasing an immune response. In some embodiments, the modulation is an increase in immune function. In some embodiments, the modulation of function leads to the expression of a synthetic immune receptor that specifically binds to TMPRSS4, such as a CAR that specifically binds to TMPRSS4. In some embodiments, the modulation of function leads to the activation of a cell comprising the system.

In some embodiments, the cell is a natural killer (NK) cell, a T cell, a CD8+ T cell, a CD4+ T cell, a primary T cell, or a T cell progenitor.

In some embodiments, the modulation of function of the cells comprising the priming receptor and CAR system as described herein leads to an increase in the cells' abilities to stimulate both native and activated T-cells, for example, by increasing cytokine or chemokine secretion by the cells expressing the priming receptor and CAR system. In some embodiments, the modulation of function enhances or increases the cells' ability to produce cytokines, chemokines, CARs, or costimulatory or activating receptors. In some embodiments, the modulation increases the T-cell stimulatory function of the cells expressing the priming receptor and CAR system, including, for example, the cells' abilities to trigger T-cell receptor (TCR) signaling, T-cell proliferation, or T-cell cytokine production.

In some embodiments, the increased immune response is secretion of cytokines and chemokines. In some embodiments, the priming receptor and CAR system induces increased expression of at least one cytokine or chemokine in a cell as compared to an isotype control cell. In some embodiments, the at least one cytokine or chemokine is selected from the group consisting of: TNFα and IFNγ. In some embodiments, the cytokine or chemokine is TNFα. In some embodiments, the cytokine or chemokine is IFNγ. In some embodiments, the cytokine or chemokine secretion is increased a between bout 1-100-fold 1, 5, 10, 20, 30, 40, 50, 60, 70, 80, 90, 100, 1-10, 10-20, 20-30, 30-40, 40-50, 50-60, 60-70, 70-80, 80-90, or 90-100 fold as compared to an untreated cell or a cell treated with an isotype control antibody. In some embodiments, the chemokine is TNFα and the secretion is increased between about 1-100-fold, 1-fold, 5-fold, 10-fold, 20-fold, 30-fold, 40-fold, 50-fold, 60-fold, 70-fold, 80-fold, 90-fold, 100-fold, 1-10-fold, 10-20-fold, 20-30-fold, 30-40-fold, 40-50-fold, 50-60-fold, 60-70-fold, 70-80-fold, 80-90-fold, or 90-100-fold as compared to an untreated cell or a cell treated with an isotype control antibody. In some embodiments, the cytokine is IFNγ and the secretion is increased between about 1-100-fold, 1-fold, 5-fold, 10-fold, 20-fold, 30-fold, 40-fold, 50-fold, 60-fold, 70-fold, 80-fold, 90-fold, 100-fold, 1-10-fold, 10-20-fold, 20-30-fold, 30-40-fold, 40-50-fold, 50-60-fold, 60-70-fold, 70-80-fold, 80-90-fold, or 90-100-fold as compared to an untreated cell or a cell treated with an isotype control antibody.

In some embodiments, the enhanced immune response is anti-tumor immune cell recruitment and activation.

In some embodiments, the cell expressing the priming receptor and CAR system induces a memory immune response as compared to an isotype control cell. In general, a memory immune response is a protective immune response upon a subsequent exposure to pathogens or antigens that the immune system encountered previously. Exemplary memory immune responses include the immune response after infection or vaccination with an antigen. In general, memory immune responses are mediated by lymphocytes such as T cells or B cells. In some embodiments, the memory immune response is a protective immune response to cancer, including cancer cell growth, proliferation, or metastasis. In some embodiments, the memory immune response inhibits, prevents, or reduces cancer cell growth, proliferation, or metastasis.

### Methods of Reducing Gene Expression

One aspect of the invention provides a method for attenuating expression of a target gene in mammalian cells, comprising introducing into the mammalian cells a recombinant nucleic acid complementary to the target gene mRNA, such as a single-stranded hairpin ribonucleic acid (shRNA), siRNA, dsRNA, or antisense oligonucleotide. In some embodiments, the recombinant nucleic acid complementary to the target gene mRNA is an shRNA. In some embodiments, the shRNA comprises self-complementary sequences of 19 to 100 nucleotides that form a duplex region, which self-complementary sequences hybridize under intracellular conditions to a target gene mRNA transcript. In some embodiments, the shRNA comprises self-complementary sequences of 22 nt. In some embodiments, the shRNA: (i) is a substrate for cleavage by a RNaseIII enzyme to produce a double-stranded RNA product, (ii) does not produce a general sequence-independent killing of the mammalian cells, and (iii) reduces expression of said target gene in a manner dependent on the sequence of said complementary regions. In some embodiments, the target gene is FAS. In some embodiments, the target gene is human FAS. In some embodiments, the target gene is PTPN2. In some embodiments, the target gene is human PTPN2. In some embodiments, the target gene is TGFBR2. In some embodiments, the target gene is human TGFBR2.

The immune cell comprising the recombinant nucleic acid can have reduced or decreased expression of a target gene selected from the group consisting of FAS, PTPN2, and TGFBR2. In some embodiments, the immune cell has reduced FAS, PTPN2, and/or TGFBR2 expression of between about 50-100%, 50-99%, 50-95%, 50-90%, 50-85%, 50-80%, 50-75%, 50-70%, 50-65%, 50-60%, 50-55%, as compared to a control cell that does not comprise the recombinant nucleic acid molecule(s). In some embodiments, the immune cell has reduced FAS expression in the immune cell by at least 50%, 55%, 60%, 65%, 75%, 80%, 85%, 90%, 95%, or 99% as compared to a control cell that does not comprise the recombinant nucleic acid molecule(s). In some embodiments, the immune cell has reduced PTPN2 expression in the immune cell by at least 50%, 55%, 60%, 65%, 75%, 80%, 85%, 90%, 95%, or 99% as compared to a control cell that does not comprise the recombinant nucleic acid molecule(s). In some embodiments, the immune cell has reduced TGFBR2 expression in the immune cell by at least 50%, 55%, 60%, 65%, 75%, 80%, 85%, 90%, 95%, or 99% as compared to a control cell that does not comprise the recombinant nucleic acid molecule(s).

In some embodiments, expression of FAS in the immune cell is reduced by at least 50%, 55%, 60%, 65%, 75%, 80%, 85%, 90%, 95%, or 99% as compared to a control cell that does not comprise the first nucleic acid. In some embodiments, the second nucleic acid is capable of reducing expression of PTPN2 in the immune cell by at least 50%, 55%, 60%, 65%, 75%, 80%, 85%, 90%, 95%, or 99% as compared to a control cell that does not comprise the second nucleic acid. In some embodiments, expression of PTPN2 in the immune cell is reduced by at least 50%, 55%, 60%, 65%, 75%, 80%, 85%, 90%, 95%, or 99% as compared to a control cell that does not comprise the second nucleic acid.

In some embodiments, the second nucleic acid is capable of reducing expression of TGFBR2 in the immune cell by at least 50%, 55%, 60%, 65%, 75%, 80%, 85%, 90%, 95%, or 99% as compared to a control cell that does not comprise the second nucleic acid. In some embodiments, expression of TGFBR2 in the immune cell is reduced by at least 50%, 55%, 60%, 65%, 75%, 80%, 85%, 90%, 95%, or 99% as compared to a control cell that does not comprise the second nucleic acid.

In some embodiments, expression of FAS, PTPN2, and/or TGFBR2 is determined by a nucleic acid assay or a protein assay. In some embodiments, the nucleic acid assay comprises at least one of polymerase chain reaction (PCR), quantitative PCR (qPCR), RT-qPCR, microarray, gene array, or RNAseq

### Methods of Editing Cells

Provided herein are methods of inserting nucleotide sequences greater than about 5 kilobases in length into the genome of a cell, in the absence of a viral vector. In some embodiments, the nucleotide sequence greater than about 5 kilobase in length can be inserted into the genome of a primary immune cell, in the absence of a viral vector

Integration of large nucleic acids, for example nucleic acids greater than 5 kilobase in size, into cells, can be limited by low efficiency of integration, off-target effects and/or loss of cell viability. Described herein are methods and compositions for achieving integration of a nucleotide sequence, for example, a nucleotide sequence greater than about 5 kilobases in size, into the genome of a cell. In some methods the efficiency of integration is increased, off-target effects are reduced and/or loss of cell viability is reduced.

The plasmid can be introduced into an immune cell with a nuclease, such as a CRISPR-associated system (Cas). The nuclease can be introduced in a ribonucleoprotein format with a guide RNA (gRNA) that targets a specific site on the genome of the immune cell. The nuclease cuts the genomic DNA at this specific site. The specific site may be a portion of the genome that encodes an endogenous immune cell receptor. Thus, cutting the genome at this site will cause the immune cell to no longer express an endogenous immune cell receptor.

The plasmid may include 5′ and 3′ homology-directed repair arms complementary to sequences at a specific site on the genome of the immune cell. The complementary sequences are on either side of the site cut by the nuclease, which allows the plasmid to be incorporated at a specified insertion site on the immune cell's genome. Once the plasmid is incorporated, the cell will express the priming receptor. However, as explained, the design of the transgene cassette ensures that non-virally delivered circuit system receptors do not express CAR until the priming receptor binds to its cognate ligand and releases the cleavable transcription factor.

Initially, an immune cell such as a T cell is activated. The immune cell or T cell may be obtained from a patient. Thus, the present disclosure provides methods in which immune cells, such as T cells, are harvested from a patient. Then, the plasmid that encodes the CAR and priming receptor are introduced into the immune cell (e.g., the T cell). Advantageously, the plasmids of the present disclosure can be introduced using electroporation. When introducing the plasmid via electroporation, the nuclease may also be introduced. By using electroporation, methods of the present disclosure avoid the use of viral vectors for introducing transgenes, which is a known bottleneck in immune cell engineering. The immune cells (e.g., the T cells) are then expanded and co-cultured to create a sufficient quantity of engineered immune cells to be used as a therapeutic treatment.

Methods for editing the genome of a cell can include a) providing a Cas9 ribonucleoprotein complex (RNP)-DNA template complex comprising: (i) the RNP, wherein the RNP comprises a Cas9 nuclease domain and a guide RNA, wherein the guide RNA specifically hybridizes to a target region of the genome of the cell, and wherein the Cas9 nuclease domain cleaves the target region to create an insertion site in the genome of the cell; and (ii) a double-stranded or single-stranded DNA template, wherein the size of the DNA template is greater than about 200 nucleotides, wherein the 5′ and 3′ ends of the DNA template comprise nucleotide sequences that are homologous to genomic sequences flanking the insertion site, and wherein the molar ratio of RNP to DNA template in the complex is from about 3:1 to about 100:1; and b) introducing the RNP-DNA template complex into the cell.

In some embodiments, the methods described herein provide an efficiency of delivery of the RNP-DNA template complex of at least about 20%, 25%, 30%, 35%, 40%, 45%, 50%, 55%, 60%, 65%, 70%, 75%, 80%, 85%, 90%, 95%, 97.5%, 99%, 99.5%, 99%, or higher. In some cases, the efficiency is determined with respect to cells that are viable after introducing the RNP-DNA template into the cell. In some cases, the efficiency is determined with respect to the total number of cells (viable or non-viable) in which the RNP-DNA template is introduced into the cell.

As another example, the efficiency of delivery can be determined by quantifying the number of genome edited cells in a population of cells (as compared to total cells or total viable cells obtained after the introducing step). Various methods for quantifying genome editing can be utilized. These methods include, but are not limited to, the use of a mismatch-specific nuclease, such as T7 endonuclease I; sequencing of one or more target loci (e.g., by sanger sequencing of cloned target locus amplification fragments); and high-throughput deep sequencing.

In some embodiments, loss of cell viability is reduced as compared to loss of cell viability after introduction of naked DNA into a cell or introduction of DNA into a cell using a viral vector. The reduction can be a reduction of at least 10%, 20%, 30%, 40%, 50%, 60%, 70%, 80%, 90%, 100% or any percentage in between these percentages. In some embodiments, off-target effects of integration are reduced as compared to off-target integration after introduction of naked DNA into a cell or introduction of DNA into a cell using a viral vector. The reduction can be a reduction of at least 10%, 20%, 30%, 40%, 50%, 60%, 70%, 80%, 90%, 100% or any percentage in between these percentages.

In some cases, the methods described herein provide for high cell viability of cells to which the RNP-DNA template has been introduced. In some cases, the viability of the cells to which the RNP-DNA template has been introduced is at least about 20%, 25%, 30%, 35%, 40%, 45%, 50%, 55%, 60%, 65%, 70%, 75%, 80%, 85%, 90%, 95%, 97.5%, 99%, 99.5%, 99%, or higher. In some cases, the viability of the cells to which the RNP-DNA template has been introduced is from about 20% to about 99%, from about 30% to about 90%, from about 35% to about 85% or 90% or higher, from about 40% to about 85% or 90% or higher, from about 50% to about 85% or 90% or higher, from about 50% to about 85% or 90% or higher, from about 60% to about 85% or 90% or higher, or from about 70% to about 85% or 90% or higher.

In the methods provided herein, the molar ratio of RNP to DNA template can be from about 3:1 to about 100:1. For example, the molar ratio can be from about 5:1 to 10:1, from about 5:1 to about 15:1, 5:1 to about 20:1; 5:1 to about 25:1; from about 8:1 to about 12:1; from about 8:1 to about 15:1, from about 8:1 to about 20:1, or from about 8:1 to about 25:1.

In some embodiments, the DNA template is at a concentration of about 2.5 pM to about 25 pM. For example, the concentration of DNA template can be about 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25 pM or any concentration in between these concentrations.

In some embodiments, the size or length of the DNA template is greater than about 4.5 kb, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, or 10 kb or any size of DNA template in between these sizes. For example, the size of the DNA template can be about 4.5 kb to about 10 kb, about 5 kb to about 10 kb, about 5 kb to about 9 kb, about 5 kb to about 8 kb, about 5 kb to about 7 kb, about 5 kb to about 6 kb, about kb 6 to about 10 kb, about 6 kb to about 9 kb, about 6 kb to about 8 kb, about 6 kb to about 7 kb, about 7 kb to about 10 kb, about 7 kb to about 9 kb, about 7 kb to about 8 kb, about 8 kb to about 10 kb, about 8 kb to about 9 kb, or about 9 kb to about 10 kb.

In some embodiments, the amount of DNA template is about 1 μg to about 10 μg. For example, the amount of DNA template can be about 1 μg to about 2 μg, about 1 μg to about 3 μg, about 1 μg to about 4 μg, about 1 μg to about 5 μg, about 1 μg to about 6 μg, about 1 μg to about 7 μg, about 1 μg to about 8 μg, about 1 μg to about 9 μg, about 1 μg to about 10 μg. In some embodiments the amount of DNA template is about 2 μg to about 3 μg, about 2 μg to about 4 μg, about 2 μg to about 5 μg, about 2 μg to about 6 μg, about 2 μg to about 7 μg, about 2 μg to about 8 μg, about 2 μg to about 9 μg, or 2 μg to about 10 μg. In some embodiments the amount of DNA template is about 3 μg to about 4 μg, about 3 μg to about 5 μg, about 3 μg to about 6 μg, about 3 μg to about 7 μg, about 3 μg to about 8 μg, about 3 μg to about 9 μg, or about 3 μg to about 10 μg. In some embodiments, the amount of DNA template is about 4 μg to about 5 μg, about 4 μg to about 6 μg, about 4 μg to about 7 μg, about 4 μg to about 8 μg, about 4 μg to about 9 μg, or about 4 μg to about 10 μg. In some embodiments, the amount of DNA template is about 5 μg to about 6 μg, about 5 μg to about 7 μg, about 5 μg to about 8 μg, about 5 μg to about 9 μg, or about 5 μg to about 10 μg. In some embodiments, the amount of DNA template is about 6 μg to about 7 μg, about 6 μg to about 8 μg, about 6 μg to about 9 μg, or about 6 μg to about 10 μg. In some embodiments, the amount of DNA template is about 7 μg to about 8 μg, about 7 μg to about 9 μg, or about 7 μg to about 10 μg. In some embodiments, the amount of DNA template is about 8 μg to about 9 μg, or about 8 μg to about 10 μg. In some embodiments, the amount of DNA template is about 9 μg to about 10 μg.

In some cases, the size of the DNA template is large enough and in sufficient quantity to be lethal as naked DNA. In some embodiments, the DNA template encodes a heterologous protein or a fragment thereof. In some embodiments, the DNA template encodes at least one protein or comprises at least one gene. In some embodiments, the DNA template encodes at least two proteins or comprises at least two genes. In some embodiments, the DNA template encodes one, two, three, four, five, six, seven, eight, nine, ten, or more proteins or comprises one, two, three, four, five, six, seven, eight, nine, ten, or more genes.

In some embodiments, the DNA template includes regulatory sequences, for example, a promoter sequence and/or an enhancer sequence to regulate expression of the heterologous protein or fragment thereof after insertion into the genome of a cell.

In some cases, the DNA template is a linear DNA template. In some cases, the DNA template is a single-stranded DNA template. In some cases, the single-stranded DNA template is a pure single-stranded DNA template. As used herein, by “pure single-stranded DNA” is meant single-stranded DNA that substantially lacks the other or opposite strand of DNA. By “substantially lacks” is meant that the pure single-stranded DNA lacks at least 100-fold more of one strand than another strand of DNA. In some embodiments, the DNA template comprises a modification at its 5′ and/or 3′ terminus, e.g., to stabilize or protect the DNA template. Exemplary modifications include closed ends, such as closed end DNA (ceDNA) or doggy bone DNA (dbDNA;Touchlight). The template DNA may comprise additional nucleotides between the modification and the 5′ or 3′ end of the DNA template.

In some cases, the RNP-DNA template complex is formed by incubating the RNP with the DNA template for less than about one minute to about thirty minutes, at a temperature of about 20° C. to about 25° C. For example, the RNP can be incubated with the DNA template for about 5 seconds, 10 seconds, 15 seconds, 20 seconds, 25 seconds, 30 seconds, 35 seconds, 40 seconds, 45 seconds, 50 seconds, 55 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes or 30 minutes or any amount of time in between these times, at a temperature of about 20° C., 21° C., 22° C., 23° C., 24° C., or 25° C. In another example, the RNP can be incubated with the DNA template for less than about one minute to about one minute, for less than about one minute to about 5 minutes, for less than about 1 minute to about 10 minutes, for about 5 minutes to 10 minutes, for about 5 minutes to 15 minutes, for about 10 to about 15 minutes, for about 10 minutes to about 20 minutes, or for about 10 minutes to about 30 minutes, at a temperature of about 20° C. to about 25° C. In some embodiments, the RNP-DNA template complex and the cell are mixed prior to introducing the RNP-DNA template complex into the cell.

In some embodiments introducing the RNP-DNA template complex comprises electroporation. Methods, compositions, and devices for electroporating cells to introduce a RNP-DNA template complex can include those described in the examples herein. Additional or alternative methods, compositions, and devices for electroporating cells to introduce a RNP-DNA template complex can include those described in WO/2006/001614 or Kim, J. A. et al. Biosens. Bioelectron. 23, 1353-1360 (2008). Additional or alternative methods, compositions, and devices for electroporating cells to introduce a RNP-DNA template complex can include those described in U.S. Patent Appl. Pub. Nos. 2006/0094095; 2005/0064596; or 2006/0087522. Additional or alternative methods, compositions, and devices for electroporating cells to introduce a RNP-DNA template complex can include those described in Li, L. H. et al. Cancer Res. Treat. 1, 341-350 (2002); U.S. Pat. Nos. 6,773,669; 7,186,559; 7,771,984; 7,991,559; 6,485,961; 7,029,916; and U.S. Patent Appl. Pub. Nos: 2014/0017213; and 2012/0088842, all of which are hereby incorporated by reference. Additional or alternative methods, compositions, and devices for electroporating cells to introduce a RNP-DNA template complex can include those described in Geng, T. et al., J. Control Release 144, 91-100 (2010); and Wang, J., et al. Lab. Chip 10, 2057-2061 (2010), all of which are hereby incorporated by reference.

In some embodiments, the Cas9 protein can be in an active endonuclease form, such that when bound to target nucleic acid as part of a complex with a guide RNA or part of a complex with a DNA template, a double strand break is introduced into the target nucleic acid. The double strand break can be repaired by NHEJ to introduce random mutations, or HDR to introduce specific mutations. Various Cas9 nucleases can be utilized in the methods described herein. For example, a Cas9 nuclease that requires an NGG protospacer adjacent motif (PAM) immediately 3′ of the region targeted by the guide RNA can be utilized. Such Cas9 nucleases can be targeted to any region of a genome that contains an NGG sequence. As another example, Cas9 proteins with orthogonal PAM motif requirements can be utilized to target sequences that do not have an adjacent NGG PAM sequence. Exemplary Cas9 proteins with orthogonal PAM sequence specificities include, but are not limited to, CFP1, those described in Nature Methods 10, 1116-1121 (2013), and those described in Zetsche et al., Cell, Volume 163, Issue 3, p759-771, 22 Oct. 2015, both of which are hereby incorporated by reference.

In some cases, the Cas9 protein is a nickase, such that when bound to target nucleic acid as part of a complex with a guide RNA, a single strand break or nick is introduced into the target nucleic acid. A pair of Cas9 nickases, each bound to a structurally different guide RNA, can be targeted to two proximal sites of a target genomic region and thus introduce a pair of proximal single stranded breaks into the target genomic region. Nickase pairs can provide enhanced specificity because off-target effects are likely to result in single nicks, which are generally repaired without lesion by base-excision repair mechanisms. Exemplary Cas9 nickases include Cas9 nucleases having a D10A or H840A mutation.

In some embodiments, the RNP comprises a Cas9 nuclease. In some embodiments, the RNP comprises a Cas9 nickase. In some embodiments, the RNP-DNA template complex comprises at least two structurally different RNP complexes. In some embodiments, the at least two structurally different RNP complexes contain structurally different Cas9 nuclease domains In some embodiments, the at least two structurally different RNP complexes contain structurally different guide RNAs. In some embodiments, wherein the at least two structurally different RNP complexes contain structurally different guide RNAs, each of the structurally different RNP complexes comprises a Cas9 nickase, and the structurally different guide RNAs hybridize to opposite strands of the target region.

In some cases, a plurality of RNP-DNA templates comprising structurally different ribonucleoprotein complexes is introduced into the cell. For example a Cas9 protein can be complexed with a plurality (e.g., 2, 3, 4, 5, or more, e.g., 2-10, 5-100, 20-100) of structurally different guide RNAs to target insertion of a DNA template at a plurality of structurally different target genomic regions.

In the methods and compositions provided herein, cells include, but are not limited to, eukaryotic cells, prokaryotic cells, animal cells, plant cells, fungal cells and the like. Optionally, the cell is a mammalian cell, for example, a human cell. The cell can be in vitro, ex vivo, or in vivo. The cell can also be a primary cell, a germ cell, a stem cell or a precursor cell. The precursor cell can be, for example, a pluripotent stem cell, or a hematopoietic stem cell. In some embodiments, the cell is a primary hematopoietic cell or a primary hematopoietic stem cell. In some embodiments, the primary hematopoietic cell is an immune cell. In some embodiments, the immune cell is a T cell. In some embodiments, the T cell is a regulatory T cell, an effector T cell, or a naïve T cell. In some embodiments, the T cell is a CD4⁺ T cell. In some embodiments, the T cell is a CD8⁺ T cell. In some embodiments, the T cell is a CD4⁺CD8⁺ T cell. In some embodiments, the T cell is a CD4⁻CD8⁻ T cell. Populations of any of the cells modified by any of the methods described herein are also provided. In some embodiments, the methods further comprise expanding the population of modified cells.

In some cases, the cells are removed from a subject, modified using any of the methods described herein and administered to the patient. In other cases, any of the constructs described herein is delivered to the patient in vivo. See, for example, U.S. Pat. No. 9,737,604 and Zhang et al. “Lipid nanoparticle-mediated efficient delivery of CRISPR/Cas9 for tumor therapy,” NPG Asia Materials Volume 9, page e441 (2017), both of which are hereby incorporated by reference.

In some embodiments, the RNP-DNA template complex is introduced into about 1×10⁵ to about 2×10⁶ cells. For example, the RNP-DNA template complex can be introduced into about 1×10⁵ to about 5×10⁵ cells, about 1×10⁵ to about 1×10⁶, 1×10⁵ to about 1.5×10⁶, 1×10⁵ to about 2×10⁶, about 1×10⁶ to about 1.5×10⁶ cells or about 1×10⁶ to about 2×10⁶.

In some cases, the methods and compositions described herein can be used for generation, modification, use, or control of recombinant T cells, such as chimeric antigen receptor T cells (CAR T cells). Such CAR T cells can be used to treat or prevent cancer, an infectious disease, or autoimmune disease in a subject. For example, in some embodiments, one or more gene products are inserted or knocked-in to a T cell to express a heterologous protein (e.g., a chimeric antigen receptor (CAR) or a priming receptor).

Genetic engineering (e.g., genome editing, nuclease-mediated editing, CRISPR/Cas9-mediated editing, etc.), engineering expression of heterologous receptors (e.g., CAR and/or TCRs), and RNAi (e.g., antisense RNA, siRNA, microRNA, shRNA, etc.) are described in International Publication Nos. WO2018232356A1, WO2019084552A1, WO2019226998A1, WO2020014235A1, WO2020123871A1, and WO2020186219A1, each of which is herein incorporated by reference for all purposes.

### Insertion Sites

Methods for editing the genome of a T cell, specifically, include a method of editing the genome of a human T cell comprise inserting a nucleic acid sequence or construct into a target region in exon 1 of the TCR-α subunit (TRAC) gene in the human T cell. In some embodiments, the target region is in exon 1 of the constant domain of TRAC gene. In other embodiments, the target region is in exon 1, exon 2 or exon 3, prior to the start of the sequence encoding the TCR-α transmembrane domain.

Methods for editing the genome of a T cell also include a method of editing the genome of a human T cell comprise inserting a nucleic acid sequence or construct into a target region in exon 1 of a TCR-β subunit (TRBC) gene in the human T cell. In some embodiments, the target region is in exon 1 of the TRBC1 or TRBC2 gene.

Methods for editing the genome of a T cell, specifically, include a method of editing the genome of a human T cell comprise inserting a nucleic acid sequence or construct into a target region of a genomic safe harbor (GSH) site.

Methods for editing the genome of a T cell also include a method of editing the genome of a human T cell comprise inserting a nucleic acid sequence or construct into a GS94 target region (locus chr11: 128340000-128350000).

In some embodiments, the target region is target region is the GS94 locus.

Gene editing therapies include, for example, vector integration and site specific integration. Site-specific integration is a promising alternative to random integration of viral vectors, as it mitigates the risks of insertional mutagenesis or insertional oncogenesis (Kolb et al. Trends Biotechnol. 2005 23:399-406; Porteus et al. Nat Biotechnol. 2005 23:967-973; Paques et al. Curr Gen Ther. 2007 7:49-66). However, site specific integration continues to face challenges such as poor knock-in efficiency, risk of insertional oncogenesis, unstable and/or anomalous expression of adjacent genes or the transgene, low accessibility (e.g., within 20 kB of adjacent genes), etc., These challenges can be addressed, in part, through the identification and use of safe harbor loci or safe harbor sites (SHS), which are sites in which genes or genetic elements can be incorporated without disruption to expression or regulation of adjacent genes.

The most widely used of the putative human safe harbor sites is the AAVS1 site on chromosome 19q, which was initially identified as a site for recurrent adenoassociated virus insertion. Other potential SHS have been identified on the basis of homology, with sites first identified in other species (e.g., the human homolog of the permissive murine Rosa26 locus) or among the growing number of human genes that appear non-essential under some circumstances. One putative SHS of this type is the CCR5 chemokine receptor gene, which, when disrupted, confers resistance to human immunodeficiency virus infection. Additional potential genomic SHS have been identified in human and other cell types on the basis of viral integration site mapping or gene-trap analyses, as was the original murine Rosa26 locus. The three top SHS, AAVS1, CCR5, and Rosa26, are in close proximity to many protein coding genes and regulatory elements. (See Sadelain, M., et al. (2012). Safe harbours for the integration of new DNA in the human genome. Nature reviews Cancer, 12 (1), 51-58, the relevant disclosures of which are herein incorporated by reference in their entirety).

The AAVS1 (also known as the PPP1R12C locus) on human chromosome 19 is a known SHS for hosting transgenes (e.g., DNA transgenes) with expected function. It is at position 19q13.42. It has an open chromatin structure and is transcription-competent. The canonical SHS locus for AAVS1 is chr19: 55,625,241-55,629,351. See Pellenz et al. “New Human Chromosomal Sites with “Safe Harbor” Potential for Targeted Transgene Insertion.” Human gene therapy vol. 30, 7 (2019): 814-828, the relevant disclosures of which are herein incorporated by reference. An exemplary AAVS1 target gRNA and target sequence are provided below:

AAVS1-gRNA sequence:
(SEQ ID NO: 837)
ggggccactagggacaggatGTTTTAGAGCTAGAAATAGCAAGTTAAAA
TAAGGCTAGTCCGTTATCAACTTGAAAAAGTGGCACCGAGTCGGTGCTT
TTTTT
AAVS1 target sequence:
(SEQ ID NO: 838)
ggggccactagggacaggat

CCR5, which is located on chromosome 3 at position 3p21.31, encodes the major co-receptor for HIV-1. Disruption at this site in the CCR5 gene has been beneficial in HIV/AIDS therapy and prompted the development of zinc-finger nucleases that target its third exon. The canonical SHS locus for CCR5 is chr3: 46,414,443-46,414,942. See Pellenz et al. “New Human Chromosomal Sites with “Safe Harbor” Potential for Targeted Transgene Insertion.” Human gene therapy vol. 30, 7 (2019): 814-828, the relevant disclosures of which are herein incorporated by reference.

The mouse Rosa26 locus is particularly useful for genetic modification as it can be targeted with high efficiency and is expressed in most cell types tested. Irion et al. 2007 (“Identification and targeting of the ROSA26 locus in human embryonic stem cells.” Nature biotechnology 25.12 (2007): 1477-1482, the relevant disclosure of which are herein incorporated by reference) identified the human homolog, human ROSA26, in chromosome 3 (position 3p25.3). The canonical SHS locus for human Rosa26 (hRosa26) is chr3: 9,415,082-9,414,043. See Pellenz et al. “New Human Chromosomal Sites with “Safe Harbor” Potential for Targeted Transgene Insertion.” Human gene therapy vol. 30, 7 (2019): 814-828, the relevant disclosures of which are herein incorporated by reference.

Additional examples of safe harbor sites are provided in Pellenz et al. “New Human Chromosomal Sites with “Safe Harbor” Potential for Targeted Transgene Insertion.” Human gene therapy vol. 30, 7 (2019): 814-828, the relevant disclosures of which are herein incorporated by reference. Examples of additional integration sites are provided in Table 9.

In some embodiments, the safe harbor sites allow for high transgene expression (sufficient to allow for transgene functionality or treatment of a disease of interest) and stable expression of the transgene over several days, weeks or months. In some embodiments, knockout of the gene at the safe harbor locus confers benefit to the function of the cell, or the gene at the safe harbor locus has no known function within the cell. In some embodiments the safe harbor locus results in stable transgene expression in vitro with or without CD3/CD28 stimulation, negligible off-target cleavage as detected by iGuide-Seq or CRISPR-Seq, less off-target cleavage relative to other loci as detected by iGuide-Seq or CRISPR-Seq, negligible transgene-independent cytotoxicity, negligible transgene-independent cytokine expression, negligible transgene-independent chimeric antigen receptor expression, negligible deregulation or silencing of nearby genes, and positioned outside of a cancer-related gene.

As used, a “nearby gene” can refer to a gene that is within about 100 kilobases (kb), about 125 kb, about 150 kb, about 175 kb, about 200 kb, about 225 kb, about 250 kb, about 275 kb, about 300 kb, about 325 kb, about 350 kb, about 375 kb, about 400 kb, about 425 kb, about 450 kb, about 475 kb, about 500 kb, about 525 kb, about 550 kb away from the safe harbor locus (integration site).

In some embodiments, the present disclosure contemplates inserts that comprise one or more transgenes. The transgene can encode a therapeutic protein, an antibody, a peptide, or any other gene of interest. The transgene integration can result in, for example, enhanced therapeutic properties. These enhanced therapeutic properties, as used herein, refer to an enhanced therapeutic property of a cell when compared to a typical immune cell of the same normal cell type. For example, a T cell having “enhanced therapeutic properties” has an enhanced, improved, and/or increased treatment outcome when compared to a typical, unmodified and/or naturally occurring T cell. The therapeutic properties of immune cells can include, but are not limited to, cell transplantation, transport, homing, viability, self-renewal, persistence, immune response control and regulation, survival, and cytotoxicity. The therapeutic properties of immune cells are also manifested by: antigen-targeted receptor expression; HLA presentation or lack thereof; tolerance to the intratumoral microenvironment; induction of bystander immune cells and immune regulation; improved target specificity with reduction; resistance to treatments such as chemotherapy.

As used herein, the term “insert size” refers to the length of the nucleotide sequence being integrated (inserted) at the target locus or safe harbor site. In some embodiments, the insert size comprises at least about 4.5 kb to about 10 kb. In some embodiments, the insert size comprises about 5000 nucleotides or more basepairs. In some embodiments, the insert size comprises up to 4.5, 4.8, 5, 6, 7, 8, 9, 10, 11, 12, 13, 14, 15, 16, 17, 18, 19, 20 kb or the sizes in between. In some embodiments, the insert size is greater than 4.5, 4.8, 5, 6, 7, 8, 9, 10, 11, 12, 13, 14, 15, 16, 17, 18, 19, 20 kb or the sizes in between. In some embodiments, the insert size is within the range of 4.5-15 kb or is any number in that range. In some embodiments, the insert size is within the range of 4.8-8.3 kb or is any number in that range. In some embodiments, the insert size is within the range of 5-8.3 kb or is any number in that range. In some embodiments, the insert size is within the range of 5-15 kb or is any number in that range. In some embodiments, the insert size is within the range of 4.5-20 kb or is any number in that range. In some embodiments, the insert size is 5-10 kb. In some embodiments, the insert size is 4.5-10, 5-10, 6-10, 7-10, 8-10, 9-10 kb. In some embodiments, the insert size is 4.5-11, 6-11, 7-11, 8-11, 9-11, or 10-11 kb. In some embodiments, the insert size is 4.5-12, 6-12, 7-12, 8-12, 9-12, 10-12, or 11-12 kb. In some embodiments, the insert size is 4.5-13, 6-13, 7-13, 8-13, 9-13, 10-13, 11-13, or 12-13 kb. In some embodiments, the insert size is 4.5-14, 6-14, 7-14, 8-14, 9-14, 10-14, 11-14, 12-14 or 13-14 kb. In some embodiments, the insert size is 4.5-15, 6-15, 7-15, 8-15, 9-15, 10-15, 11-15, 12-15, 13-15, or 14-15 kb. In some embodiments, the insert size is 4.5-16, 6-16, 7-16, 8-16, 9-16, 10-16, 11-16, 12-16, 13-16, 14-16 or 15-16 kb. In some embodiments, the insert size is 4.5-17, 6-17, 7-17, 8-17, 9-17, 10-17, 11-17, 12-17, 13-17, or 14-17, 15-17 or 16-17 kb. In some embodiments, the insert size is 4.5-18, 6-18, 7-18, 8-18, 9-18, 10-18, 11-18, 12-18, 13-18, 14-18, 15-18, 16-18 or 17-18 kb. In some embodiments, the insert size is 4.5-19, 6-19, 7-19, 8-19, 9-19, 10-19, 11-19, 12-19, 13-19, 14-19, 15-19, 16-19, 17-19, or 18-19 kb. In some embodiments, the insert size is 4.5-20, 6-20, 7-20, 8-20, 9-20, 10-20, 11-20, 12-20, 13-20, 14-20, 15-20, 16-20, 17-20, 18-20, or 19-20 kb.

The inserts of the present disclosure refer to nucleic acid molecules or polynucleotide inserted at a target locus or safe harbor site. In some embodiments, the nucleotide sequence is a DNA molecule, e.g., genomic DNA, or comprises deoxy-ribonucleotides. In some embodiments, the insert comprises a smaller fragment of DNA, such as a plastid DNA, mitochondrial DNA, or DNA isolated in the form of a plasmid, a fosmid, a cosmid, a bacterial artificial chromosome (BAC), a yeast artificial chromosome (YAC), and/or any other sub-genome segment of DNA. In some embodiments, the insert is an RNA molecule or comprises ribonucleotides. The nucleotides in the insert are contemplated as naturally occurring nucleotides, non-naturally occurring, and modified nucleotides. Nucleotides may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications. The polynucleotides can be in any topological conformation, including single-stranded, double-stranded, partially duplexed, triplexed, hairpinned, circular conformations, and other three-dimension conformations contemplated in the art.

The inserts can have coding and/or non-coding regions. The insert can comprises a non-coding sequence (e.g., control elements, e.g., a promoter sequence). In some embodiments, the insert encodes transcription factors. In some embodiments, the insert encodes an antigen binding receptors such as single receptors, T-cell receptors (TCRs), priming receptors, CARs, mAbs, etc. In some embodiments, the insert is a human sequence. In some embodiments, the insert is chimeric. In some embodiments, the insert is a multi-gene/multi-module therapeutic cassette. A multi-gene/multi-module therapeutic cassette refers to an insert or cassette having one or more than one receptor (e.g., synthetic receptors), other exogenous protein coding sequences, non-coding RNAs, transcriptional regulatory elements, and/or insulator sequences, etc.

In some embodiments, the nucleic acid sequence is inserted into the genome of the T cell via non-viral delivery. In non-viral delivery methods, the nucleic acid can be naked DNA, or in a non-viral plasmid or vector. Non-viral delivery techniques can be site-specific integration techniques, as described herein or known to those of ordinary skill in the art. Examples of site-specific techniques for integration into the safe harbor loci include, without limitation, homology-dependent engineering using nucleases and homology independent targeted insertion using Cas9 or other CRISPR endonucleases.

In some embodiments, the insert is integrated at a safe harbor site by introducing into the engineered cell, (a) a targeted nuclease that cleaves a target region in the safe harbor site to create the insertion site; and (b) the nucleic acid sequence (insert), wherein the insert is incorporated at the insertion site by, e.g., HDR. Examples of non-viral delivery techniques that can be used in the methods of the present disclosure are provided in U.S. application Ser. Nos. 16/568,116 and 16/622,843, the relevant disclosures of which are herein incorporated by reference in their entirety. In some embodiments, the genomic safe harbor is the GS94 target region (chr11: 128340000-128350000). In some embodiments, the genomic safe harbor is the GS102 target region (chr15: 92830000-92840000).

Examples of integration sites contemplated are provided in Table 22. In some embodiments, an integration site comprises any site and/or sgRNA selected from Table 22.

TABLE 22
sgRNA sequences

| | SEQ | | | | |
|---|---|---|---|---|---|
| | ID | | sgRNA start coor | sgRNA | Integration |
| sgRNA ID | NO: | sgRNA Sequence | GRCH38 | Target Loci | Site |
| sgRNA_1 | 839 | GCACCTGAATACCACGCCTG | chr16:88811818 | APRT | APRT |
| sgRNA_2 | 840 | CGCCTGCGATGTAGTCGATG | chr16:88811551 | APRT | APRT |
| sgRNA_3 | 841 | CAGGACGGGCGAGATGTCCC | chr16:88811640 | APRT | APRT |
| sgRNA_4 | 842 | CTGAATCTTTGGAGTACCTG | chr15:44715425 | B2M | B2M |
| sgRNA_5 | 843 | GGCCACGGAGCGAGACATCT | chr15:44711550 | B2M | B2M |
| sgRNA_6 | 844 | AAGTCAACTTCAATGTCGGA | chr15:44715515 | B2M | B2M |
| sgRNA_7 | 845 | GCTTGGAGGCCTGATCAGCG | chr19:36141111 | CAPNS1 | CAPNS1 |
| sgRNA_8 | 846 | CTTATCTCTTCGCAGCGAGG | chr19:36142301 | CAPNS1 | CAPNS1 |
| sgRNA_9 | 847 | CACACATTACTCCAACATTG | chr19:36142676 | CAPNS1 | CAPNS1 |
| sgRNA_10 | 848 | TTCCGCAAAATAGAGCCCCA | chr3:105746019 | CBLB | CBLB |
| sgRNA_11 | 849 | TGCACAGAACTATCGTACCA | chr3:105751622 | CBLB | CBLB |
| sgRNA_12 | 850 | GCAATAAGACTCTTTAAAGA | chr3:105853470 | CBLB | CBLB |
| sgRNA_13 | 851 | CAAAGAGATTACGAATGCCT | chr1:116754658 | CD2 | CD2 |
| sgRNA_14 | 852 | CAAGGCACCCCAGGTTTCCA | chr1:116754663 | CD2 | CD2 |
| sgRNA_15 | 853 | TTACGAATGCCTTGGAAACC | chr1:116754666 | CD2 | CD2 |
| sgRNA_16 | 854 | CAGAGACGCATCTGACCCTC | chr11:118315540 | CD3E | CD3E |
| sgRNA_17 | 855 | CATGCAGTTCTCACACACTG | chr11:118313715 | CD3E | CD3E |
| sgRNA_18 | 856 | GTGTGAGAACTGCATGGAGA | chr11:118313715 | CD3E | CD3E |
| sgRNA_19 | 857 | TCTCATTTCAGGAAACCACT | chr11:118349748 | CD3G | CD3G |
| sgRNA_20 | 858 | AGTCATACACCTTAACCAAG | chr11:118349754 | CD3G | CD3G |
| sgRNA_21 | 859 | TTCAAGGAAACCAGTTGAGG | chr11:118352458 | CD3G | CD3G |
| sgRNA_22 | 860 | GAGCCTTGCCTGGAAATCTG | chr11:61118177 | CD5 | CD5 |
| sgRNA_23 | 861 | AAGCGTCAAAAGTCTGCCAG | chr11:61118324 | CD5 | CD5 |
| sgRNA_24 | 862 | CGTTCCAACTCGAAGTGCCA | chr11:61118121 | CD5 | CD5 |
| sgRNA_25 | 863 | GAGCGACTGGGACACGGTGA | chr9:136866246 | EDF1 | EDF1 |
| sgRNA_26 | 864 | GCTGCGCAAGAAGGGCCCTA | chr9:136866211 | EDF1 | EDF1 |
| sgRNA_27 | 865 | TTGTTCTGGCCAGCAGCCCC | chr9:136863433 | EDF1 | EDF1 |
| sgRNA_28 | 866 | CTTCCAGAGCCACATCATCG | chr19:48965791 | FTL | FTL |
| sgRNA_29 | 867 | GGGACTCACCAGAGAGAGGT | chr19:48965601 | FTL | FTL |
| sgRNA_30 | 868 | CGGTCGAAATAGAAGCCCTA | chr19:48965770 | FTL | FTL |
| sgRNA_31 | 869 | AAAAGGATATTGTGCAACTG | chr10:87933015 | PTEN | PTEN |
| sgRNA_32 | 870 | TGTGCATATTTATTACATCG | chr10:87933183 | PTEN | PTEN |
| sgRNA_33 | 871 | TTTGTGAAGATCTTGACCAA | chr10:87933087 | PTEN | PTEN |
| sgRNA_34 | 872 | TGTCATGCTGAACCGCATTG | chr18:12830972 | PTPN2 | PTPN2 |
| sgRNA_35 | 873 | CCACTCTATGAGGATAGTCA | chr18:12859219 | PTPN2 | PTPN2 |
| sgRNA_36 | 874 | TTGACATAGAAGAGGCACAA | chr18:12836828 | PTPN2 | PTPN2 |
| sgRNA_37 | 875 | GAGTACTACACTCAGCAGCA | chr12:6952098 | PTPN6 | PTPN6 |
| sgRNA_38 | 876 | TCACGCACAAGAAACGTCCA | chr12:6954872 | PTPN6 | PTPN6 |
| sgRNA_39 | 877 | AGGTCTCGGTGAAACCACCT | chr12:6951610 | PTPN6 | PTPN6 |
| sgRNA_40 | 878 | AGCATTATCCAAAGAGTCCG | chr1:198696873 | PTPRC | PTPRC |
| sgRNA_41 | 879 | ATATTAATTCTTACCAGTGG | chr1:198692370 | PTPRC | PTPRC |
| sgRNA_42 | 880 | AGCTTTAAATCAAGGTTCAT | chr1:198756176 | PTPRC | PTPRC |
| sgRNA_43 | 881 | ATCCCGAGCCCTAAGGTGCA | chr11:67436325 | PTPRCAP | PTPRCAP |
| sgRNA_44 | 882 | GGCAGCGCGGAGGACAGCGT | chr11:67436285 | PTPRCAP | PTPRCAP |
| sgRNA_45 | 883 | CTCAGGGGGCTACTACCACC | chr11:67436170 | PTPRCAP | PTPRCAP |
| sgRNA_46 | 884 | GTCACCGACGAGACCAGAAG | chr5:82277810 | RPS23 | RPS23 |
| sgRNA_47 | 885 | GTCGTGGACTTCGTACTGCT | chr5:82277843 | RPS23 | RPS23 |
| sgRNA_48 | 886 | TAATTTTTAGGCAAGTGTCG | chr5:82277860 | RPS23 | RPS23 |
| sgRNA_49 | 887 | TTAGCTGTTAGACTTGAATA | chr14:51993810 | RTRAF | RTRAF |
| sgRNA_50 | 888 | CGAGAGCCGTCAACTTGCGT | chr14:51989652 | RTRAF | RTRAF |
| sgRNA_51 | 889 | CGGCTTCAACTGCAAAGGTG | chr14:51989700 | RTRAF | RTRAF |
| sgRNA_52 | 890 | TATGAAAAAGCAGAGCGACT | chr15:43793025 | SERF2 | SERF2 |
| sgRNA_53 | 891 | TCTGGCGGGCGAGCTCACGC | chr15:43792989 | SERF2 | SERF2 |
| sgRNA_54 | 892 | CTCACGCTGGTTACCGCCTA | chr15:43792977 | SERF2 | SERF2 |
| sgRNA_55 | 893 | AAAGATTACGAACTTCCCTG | chr12:46207559 | SLC38A1 | SLC38A1 |
| sgRNA_56 | 894 | GTTAAAAACAGACATGCCTA | chr12:46229232 | SLC38A1 | SLC38A1 |
| sgRNA_57 | 895 | ATGCCTAAGGAGGTTGTACC | chr12:46229246 | SLC38A1 | SLC38A1 |
| sgRNA_58 | 896 | CTCCAGGTATCCCATCGAAA | chr18:47869418 | SMAD2 | SMAD2 |
| sgRNA_59 | 897 | CACCAAATACGATAGATCAG | chr18:47870532 | SMAD2 | SMAD2 |
| sgRNA_60 | 898 | TGGCGGCGTGAATGGCAAGA | chr18:47896729 | SMAD2 | SMAD2 |
| sgRNA_61 | 899 | TAGGATGGTAGCACACAACC | chr16:11255478 | SOCS1 | SOCS1 |
| sgRNA_62 | 900 | CAGCAGCAGAGCCCCGACGG | chr16:11255432 | SOCS1 | SOCS1 |
| sgRNA_63 | 901 | CGGCGTGCGAACGGAATGTG | chr16:11255296 | SOCS1 | SOCS1 |
| sgRNA_64 | 902 | TATAGACGCTGCCCGACGTC | chr15:40038895 | SRP14 | SRP14 |
| sgRNA_65 | 903 | TCCAAAGAAGGGTACTGTGG | chr15:40038368 | SRP14 | SRP14 |
| sgRNA_66 | 904 | ACAGTACCCTTCTTTGGAAT | chr15:40038358 | SRP14 | SRP14 |
| sgRNA_67 | 905 | GCGACGGGCGCATCTACGTG | chr12:120469572 | SRSF9 | SRSF9 |
| sgRNA_68 | 906 | CCCGACCTCCATAAGTCCTG | chr12:120465700 | SRSF9 | SRSF9 |
| sgRNA_69 | 907 | GGGGTCCTCGAAGCGCACGA | chr12:120469426 | SRSF9 | SRSF9 |
| sgRNA_70 | 908 | TGCTCTGTTTAGAAGATGAC | chr5:32591641 | SUB1 | SUB1 |
| sgRNA_71 | 909 | ATATTCTTTTCTAGTTAAAG | chr5:32591566 | SUB1 | SUB1 |
| sgRNA_72 | 910 | CCTGTAAAGAAACAAAAGAC | chr5:32591614 | SUB1 | SUB1 |
| sgRNA_73 | 911 | TGGAGAAAGACGTAACTTCG | chr4:105234315 | TET2 | TET2 |
| sgRNA_74 | 912 | TCTGCCCTGAGGTATGCGAT | chr4:105234747 | TET2 | TET2 |
| sgRNA_75 | 913 | ATTCCGCTTGGTGAAAACGA | chr4:105235656 | TET2 | TET2 |
| sgRNA_76 | 914 | CAGGCACAATAGAAACAACG | chr3:114295571 | TIGIT | TIGIT |
| sgRNA_77 | 915 | CCATTTGTAATGCTGACTTG | chr3:114295700 | TIGIT | TIGIT |
| sgRNA_78 | 916 | CTGGGTCACTTGTGCCGTGG | chr3:114295634 | TIGIT | TIGIT |
| sgRNA_79 | 917 | GTCAGGGTTCTGGATATCTG | chr14:22547508 | TRAC | TRAC |
| sgRNA_80 | 918 | TGGATTTAGAGTCTCTCAGC | chr14:22547541 | TRAC | TRAC |
| sgRNA_81 | 919 | CTGCGGCTGTGGTCCAGCTG | chr14:22550661 | TRAC | TRAC |
| sgRNA_82 | 920 | ACAAAACTGTGCTAGACATG | chr14:22547658 | TRAC | TRAC |
| sgRNA_83 | 921 | TTCTTCCCCAGCCCAGGTAA | chr14:22547778 | TRAC | TRAC |
| sgRNA_84 | 922 | CGTCATGAGCAGATTAAACC | chr14:22550625 | TRAC | TRAC |
| sgRNA_85 | 923 | GAGAGCGCCTGCGACCCGAG | chr19:58544980 | TRIM28 | TRIM28 |
| sgRNA_86 | 924 | CCAGCGGGTGAAGTACACCA | chr19:58544869 | TRIM28 | TRIM28 |
| sgRNA_87 | 925 | GGAGCGCTTTTCGCCGCCAG | chr19:58544839 | TRIM28 | TRIM28 |
| sgRNA_88 | 926 | TGAGGCCTGGACCTTATGCA | chr10:33134193 | chr10:33130000- | desert_1 |
| | | | | 33140000 | (GS88) |
| sgRNA_89 | 927 | CCTGGTGGAGTGAACCATGA | chr10:33132917 | chr10:33130000- | desert_1 |
| | | | | 33140000 | (GS89) |
| sgRNA_90 | 928 | CAAGCACTTAGGTTCCCCTG | chr10:33134633 | chr10:33130000- | desert_1 |
| | | | | 33140000 | (GS90) |
| sgRNA_91 | 929 | GGTCTCCCTACAATTCAGCG | chr10:72294568 | chr10:72290000- | desert_2 |
| | | | | 72300000 | (GS91) |
| sgRNA_92 | 930 | CACAGCGCGTGACTGCAATG | chr10:72298268 | chr10:72290000- | desert_2 |
| | | | | 72300000 | (GS92) |
| sgRNA_93 | 931 | TCTGGGGCACCAATTCTAGG | chr10:72292786 | chr10:72290000- | desert_2 |
| | | | | 72300000 | (GS93) |
| sgRNA_94 | 932 | GAGCCATGCTTGGCTTACGA | chr11:128342576 | chr11:128340000- | desert_3 |
| | | | | 128350000 | (GS94) |
| sgRNA_95 | 933 | GTACAAGTACTTATCTCATG | chr11:128343592 | chr11:128340000- | desert_3 |
| | | | | 128350000 | (GS95) |
| sgRNA_96 | 934 | GAGATAACAACATAACAACA | chr11:128347170 | chr11:128340000- | desert_3 |
| | | | | 128350000 | (GS96) |
| sgRNA_97 | 935 | CATATTCCATAGTCTTTGGG | chr11:65425000 | chr11:65425000- | desert_4 |
| | | | | 65427000 | (GS97) |
| | | | | (NEAT1) | |
| sgRNA_98 | 936 | CTGCCCCTTAGCAACTTAGG | chr11:65425507 | chr11:65425000- | desert_4 |
| | | | | 65427000 | (GS98) |
| | | | | (NEAT1) | |
| sgRNA_99 | 937 | TGTTTAAAAATATGTTGACA | chr11:65426264 | chr11:65425000- | desert_4 |
| | | | | 65427000 | (GS99) |
| | | | | (NEAT1) | |
| sgRNA_100 | 938 | CCAGGAATGGAAACTCACGC | chr15:92830315 | chr15:92830000- | desert_5 |
| | | | | 92840000 | (GS100) |
| sgRNA_101 | 939 | GAGGCCGCTGAATTAACCCG | chr15:92831850 | chr15:92830000- | desert_5 |
| | | | | 92840000 | (GS101) |
| sgRNA_102 | 940 | ATACACGCACACTTGCAGAA | chr15:92831131 | chr15:92830000- | desert_5 |
| | | | | 92840000 | (GS102) |
| sgRNA_103 | 941 | GAGCAGACAGAAACCCAGGG | chr16:11225670 | chr16:11220000- | desert_6 |
| | | | | 11230000 | (GS103) |
| sgRNA_104 | 942 | TGAGTCTCCAAACAGAACAG | chr16:11226284 | chr16:11220000- | desert_6 |
| | | | | 11230000 | (GS104) |
| sgRNA_105 | 943 | TAATATCACTGACTTCACGG | chr16:11225029 | chr16:11220000- | desert_6 |
| | | | | 11230000 | (GS105) |
| sgRNA_106 | 944 | TACACACAATGTAAGCAGCA | chr2:87467461 | chr2:87460000- | desert_7 |
| | | | | 87470000 | (GS106) |
| sgRNA_107 | 945 | GGGAGCTCAATTCGAAACCA | chr2:87468809 | chr2:87460000- | desert_7 |
| | | | | 87470000 | (GS107) |
| sgRNA_108 | 946 | TTGGACAGGTGAGACAGTCG | chr2:87467001 | chr2:87460000- | desert_7 |
| | | | | 87470000 | (GS108) |
| sgRNA_109 | 947 | AAGCTCACTCAGATAGTGTG | chr3:186511316 | chr3:186510000- | desert_8 |
| | | | | 186520000 | (GS109) |
| sgRNA_110 | 948 | CAGGAGAACCACCTTACACG | chr3:186515260 | chr3:186510000- | desert_8 |
| | | | | 186520000 | (GS110) |
| sgRNA_111 | 949 | GGACAGACCCTGATTCACAA | chr3:186519655 | chr3:186510000- | desert_8 |
| | | | | 186520000 | (GS111) |
| sgRNA_112 | 950 | ACATGGCAGTCTATGAACAG | chr3:59451154 | chr3:59450000- | desert_9 |
| | | | | 59460000 | (GS112) |
| sgRNA_113 | 951 | CCTATAGAGAGTACTACTTG | chr3:59456416 | chr3:59450000- | desert_9 |
| | | | | 59460000 | (GS113) |
| sgRNA_114 | 952 | CCAACCGGGTCTTCATTACG | chr3:59457029 | chr3:59450000- | desert_9 |
| | | | | 59460000 | (GS114) |
| sgRNA_115 | 953 | TCAAGCGTAGAGTTCCGAGT | chr8:127993006 | chr8:127980000- | desert_10 |
| | | | | 128000000 | (GS115) |
| sgRNA_116 | 954 | TCATGCAATTATGGACCCAG | chr8:127994663 | chr8:127980000- | desert_10 |
| | | | | 128000000 | (GS116) |
| sgRNA_117 | 955 | CGGGAAAGTGACTGGCCATG | chr8:127996766 | chr8:127980000- | desert_10 |
| | | | | 128000000 | (GS117) |
| sgRNA_118 | 956 | TGAGATTGAAATCAAATCGG | chr9:7974159 | chr9:7970000- | desert_11 |
| | | | | 7980000 | (GS118) |
| sgRNA_119 | 957 | TATGCAATATTCATCACGCG | chr9:7977914 | chr9:7970000- | desert_11 |
| | | | | 7980000 | (GS119) |
| sgRNA_120 | 958 | AATGTGTTAAATCAAATGCA | chr9:7976895 | chr9:7970000- | desert_11 |
| | | | | 7980000 | (GS120) |

### CRISPR-Cas Editing

One effective example of gene editing is the CRISPR-Cas approach (e.g., CRISPR-Cas9). This approach incorporates the use of a guide polynucleotide (e.g., guide ribonucleic acid or gRNA) and a Cas endonuclease (e.g., Cas9 endonuclease).

The guide polynucleotide includes a first nucleotide sequence domain (also referred to as a variable targeting domain or VT domain) that is complementary to a nucleotide sequence in the target DNA, and a second nucleotide that interacts with a Cas endonuclease polypeptide. It can be a double molecule (also referred to as a double-stranded guide polynucleotide) comprising a sequence domain (referred to as a Cas endonuclease recognition domain or CER domain). The CER domain of this double molecule guide polynucleotide comprises two separate molecules that hybridize along the complementary region. The two separate molecules can be RNA sequences, DNA sequences and/or RNA-DNA combination sequences.

Genome editing using CRISPR-Cas approaches relies on the repair of site-specific DNA double-strand breaks (DSBs) induced by the RNA-guided Cas endonuclease (e.g., Cas 9 endonuclease). Homology-directed repair (HDR) of these DSBs enables precise editing of the genome by introducing defined genomic changes, including base substitutions, sequence insertions, and deletions. Conventional HDR-based CRISPR/Cas9 genome-editing involves transfecting cells with Cas9, gRNA and donor DNA containing homologous arms matching the genomic locus of interest.

HITI (homology independent targeted insertion) uses a non-homologous end joining (NHEJ)-based homology-independent strategy and the method can be more efficient than HDR. Guide RNAs (gRNAs) target the insertion site. For HITI, donor plasmids lack homology arms and DSB repair does not occur through the HDR pathway. The donor polynucleotide construct can be engineered to include Cas9 cleavage site(s) flanking the gene or sequence to be inserted. This results in Cas9 cleavage at both the donor plasmid and the genomic target sequence. Both target and donor have blunt ends and the linearized donor DNA plasmid is used by the NHEJ pathway resulting integration into the genomic DSB site. (See, for example, Suzuki, K., et al. (2016). In vivo genome editing via CRISPR/Cas9 mediated homology-independent targeted integration. Nature, 540 (7631), 144-149, the relevant disclosures of which are herein incorporated in their entirety).

Methods for conducing gene editing using CRISPR-Cas approaches are known to those of ordinary skill in the art. (See, for example, US Application Nos. U.S. Ser. No. 16/312,676, U.S. Ser. No. 15/303,722, and U.S. Ser. No. 15/628,533, the disclosures of which are hereby incorporated by reference in their entireties). Additionally, uses of endonucleases for inserting transgenes into safe harbor loci are described, for example, in U.S. Ser. No. 13/036,343, the disclosure of which is herein incorporated by reference in their entirety.

The guide RNAs and/or mRNA (or DNA) encoding an endonuclease can be chemically linked to one or more moieties or conjugates that enhance the activity, cellular distribution, or cellular uptake of the oligonucleotide. Non-limiting examples of such moieties include lipid moieties such as a cholesterol moiety, cholic acid, a thioether, a thiocholesterol, an aliphatic chain (e.g., dodecandiol or undecyl residues), a phospholipid, e.g., di-hexadecyl-rac-glycerol or triethylammonium 1,2-di-O-hexadecyl-rac-glycero-3-H-phosphonate, a polyamine or a polyethylene glycol chain, adamantane acetic acid, a palmityl moiety and an octadecylamine or hexylamino-carbonyl-t oxycholesterol moiety. See for example US Patent Publication No. 20180127786, the disclosure of which is herein incorporated by reference in its entirety.

### Therapeutic Applications

For therapeutic applications, the engineered cells, populations thereof, or compositions thereof are administered to a subject, generally a mammal, generally a human, in an effective amount. The engineered cells may be administered to a subject by infusion (e.g., continuous infusion over a period of time) or other modes of administration known to those of ordinary skill in the art.

The engineered cells provided herein not only find use in gene therapy but also in non-pharmaceutical uses such as, e.g., production of animal models and production of recombinant cell lines expressing a protein of interest.

The engineered cells of the present disclosure can be any cell, generally a mammalian cell, generally a human cell that has been modified by integrating a transgene at a safe harbor locus described herein. Exemplary cells are provided in the Recombinant Cells section.

The engineered cells, compositions and methods of the present disclosure are useful for therapeutic applications such as CAR T cell therapy and TCR T cell therapy. In some embodiments, the insertion of a sequence encoding a transgene within a safe harbor locus maintains the TCR expression relative to instances when there is no insertion and enables transgene expression while maintaining TCR function.

In some embodiments, the present disclosure provides methods of treating a subject in need of treatment by administering to the subject a composition comprising any of the engineered cells described herein. In some embodiments, administration of the engineered cell composition results in a desired pharmacological and/or physiological effect. That effect can be partial or complete cure of the disease and/or adverse effects resulting from the disease. In some embodiments, treatment encompasses any treatment of a disease in a subject (e.g., mammal, e.g., human). Further, treatment may stabilize or reduce undesirable clinical symptoms in subjects (e.g., patients). The cells provided herein populations thereof, or compositions thereof may be administered during or after the occurrence of the disease.

In certain embodiments, the subject has a disease, condition, and/or injury that can be treated and/or ameliorated by cell therapy. In some embodiments, the subject in need of cell therapy is a subject having an injury, disease, or condition, thereby causing cell therapy (e.g., therapy in which cellular material is administered to the subject). However, it is contemplated that it is possible to treat, ameliorate and/or reduce the severity of at least one symptom associated with the injury, disease or condition.

### Method of Administration

An effective amount of the immune cell comprising the system may be administered for the treatment of cancer. The appropriate dosage of the immune cell comprising the system may be determined based on the type of cancer to be treated, the type of the immune cell comprising the system, the severity and course of the cancer, the clinical condition of the subject the subject's clinical history and response to the treatment, and the discretion of the attending physician.

### Pharmaceutical Compositions

The engineered recombinant cells provided herein can be administered as part of a pharmaceutical compositions. These compositions can comprise, in addition to one or more of the recombinant cells, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g., oral, intravenous, cutaneous or subcutaneous, nasal, intramuscular, intraperitoneal routes. The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

Various modes of administering the additional therapeutic agents are contemplated herein. In some embodiments, the additional therapeutic agent is administered by any suitable mode of administration.

A composition can be administered alone or in combination with other treatments, either simultaneously or sequentially dependent upon the condition to be treated.

### Kits and Articles of Manufacture

The present application provides kits comprising any one or more of the system or cell compositions described herein along with instructions for use. The instructions for use can be present in the kits as a package insert, in the labeling of the container of the kit or components thereof, or can be in digital form (e.g., on a CD-ROM, via a link on the internet). A kit can include one or more of a genome-targeting nucleic acid, a polynucleotide encoding a genome-targeting nucleic acid, a site-directed polypeptide, and/or a polynucleotide encoding a site-directed polypeptide. Additional components within the kits are also contemplated, for example, buffer (such as reconstituting buffer, stabilizing buffer, diluting buffer), and/or one or more control vectors.

In some embodiments, the kits further contain a component selected from any of secondary antibodies, reagents for immunohistochemistry analysis, pharmaceutically acceptable excipient and instruction manual and any combination thereof. In one specific embodiment, the kit comprises a pharmaceutical composition comprising any one or more of the antibody compositions described herein, with one or more pharmaceutically acceptable excipients.

The present application also provides articles of manufacture comprising any one of the antibody compositions or kits described herein. Examples of an article of manufacture include vials (including sealed vials).

### EXAMPLES

### Example 1: TMPRSS4 Antibody Discovery

This example discloses the identification and selection of antigen binding proteins (e.g., antibodies or antigen binding fragments thereof such as single chain variable fragments (scFv)) that bind to TMPRSS4 using cell-based phage display. The SuperHuman2.0 Library from Charles River was used to discover the antibodies targeting human TMPRSS4 by iterative binding of phage to the cell surface expression TMPRSS4. The antibodies obtained specifically bind to engineered HEK293T with full length TMPRSS4 over-expression.

### Materials and Methods

### Cell Line Engineering for TMPRSS4 Expression

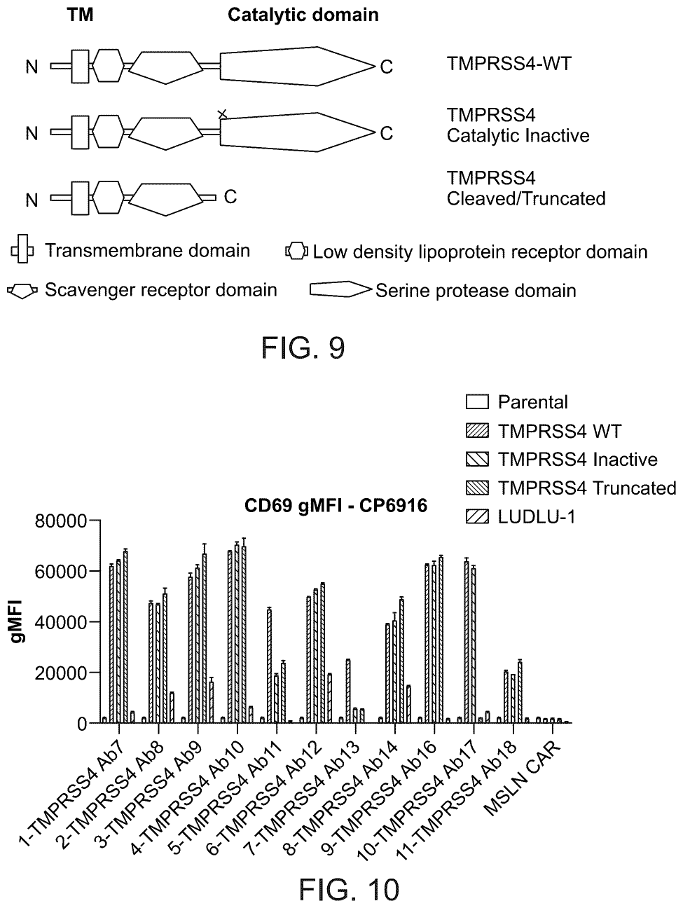

HEK293T was selected as a parental cell line. TMPRSS4 full-length, TMPRSS4 truncated format with a 150 amino acid residue ECD, and TMPRSS4 catalytic inactive format (comprising a D290A mutation) were transfected individually into HEK293T cells (FIG. 9). Separate HEK293T cell lines were generated for each of the three different forms of TMPRSS4. TMPRSS4 expression in the engineered HEK293T cells was validated by flow cytometry.

### Cell-Based Phage Selection

Four iterative rounds of phage selection were performed against the HEK293T cells engineered to overexpress human TMPRSS4 with the SuperHuman2.0 Library. This library contains fully human scFvs in a single VH-VL orientation with the (G₄S)₃ linker (SEQ ID NO: 819). The phage library was incubated with the cells, and non-binding phage was removed by washing, followed by elution of specifically bound phage. The enrichment of antibodies against TMPRSS4 was evaluated by polyclonal phage flow cytometry against HEK293T-TMPRSS4.

### Monoclonal Phage Screening and Positive Hits Sequencing

Monoclonal phages were prepared, screened for binding to HEK293T-TMPRSS4 cells by flow cytometry. Monoclonal phage clones were incubated with HEK293T-TMPRSS4 cells, the cells were washed and then incubated with a PE conjugated mouse anti-M13 mAb, and washed again before being analyzed by flow cytometry. The positive candidates were selected based on cytometry results, then followed by next-gen sequencing of the positive hits.

### Binding Activity Validation

To validate the binding activity of the positive hits to TMPRSS4, 5 candidate VH/VL pairs were cloned into mammalian expression vectors containing the mouse IgG2a constant domains. The resulting chimeric mlgG2a antibodies were transiently expressed from CHO cultures and purified via affinity chromatography. TMPRSS4-expressing HEK293T cell lines were used to assess the binding of recombinant antibodies to cell surface expressed TMPRSS4. Briefly, HEK293T parental and HEK293T-TMPRSS4 engineered cells were first blocked with a human Fc blocker for 10 min at room temperature, stained for 30 min on ice with recombinant antibodies in an 8-point serial dilution, and washed 3 times with BD Stain Buffer. Subsequently, cells were stained for 30 min on ice with anti-mouse IgG2a-PE secondary antibody. Cell surface binding was measured on an Attune flow cytometer.

### Results

### Cell Line Engineering for TMPRSS4 Expression

The different formats of TMPRSS4 were expressed on the surface of HEK293T cells with at least a 2 log shift compared to parental cell lines. The wild type full length TMPRSS4 is cell line CL681, the truncated TMPRSS4 is cell line CL610 and the catalytical inactive TMPRSS4 D290A is cell line CL612 (data not shown).

### Phage Antigen Binding Protein Enrichment Validation

Four iterative rounds of phage selection were performed against HEK293-TMPRSS4 (cell line CL681). The enrichment of antibodies against TMPRSS4 was then evaluated by polyclonal phage flow cytometry. Enrichment was observed from round 3 panning, and the phage antibodies were further enriched after round 4 (data not shown).

### The Monoclonal Phage Screening Results and Unique Positive Hits Sequence

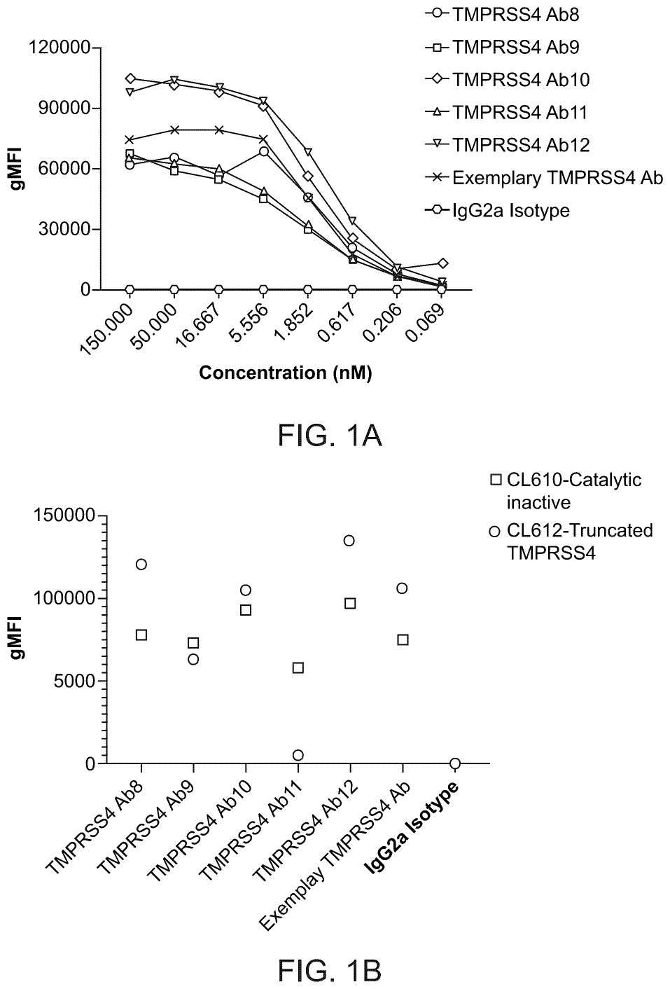

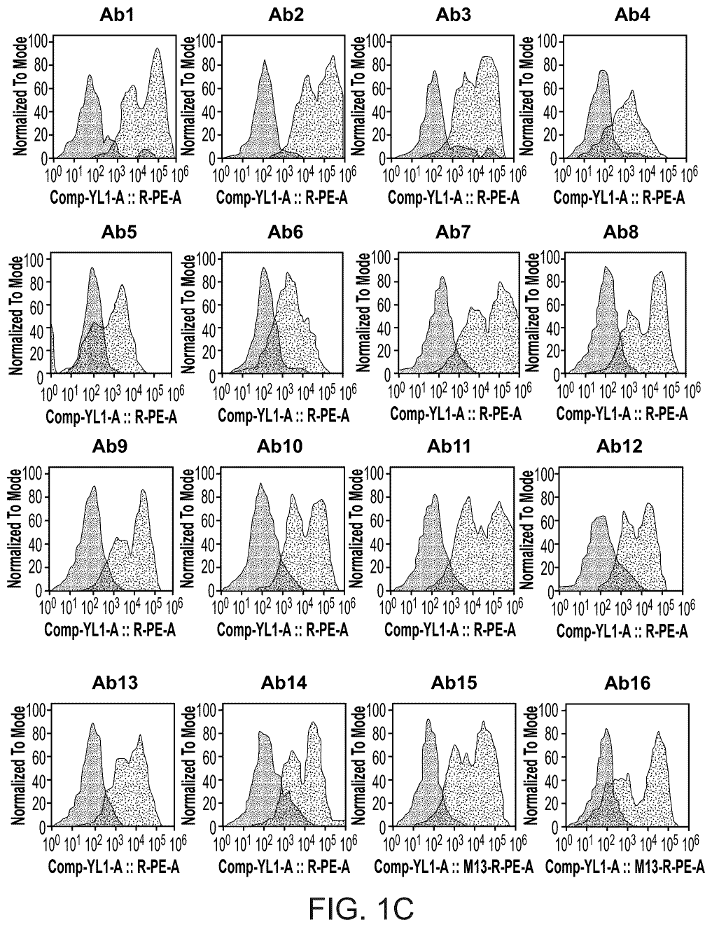

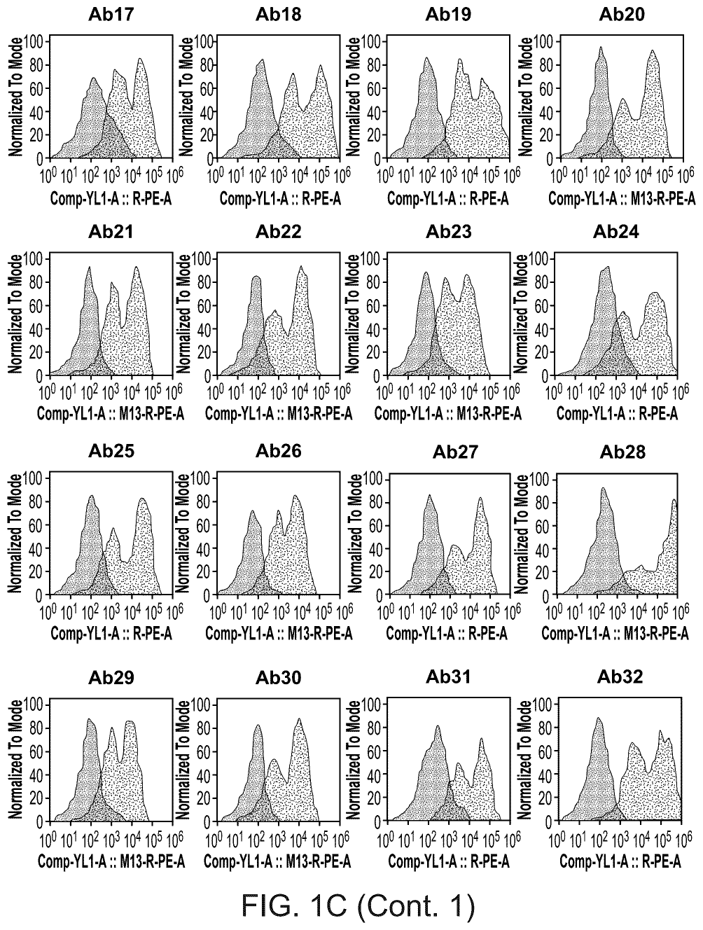

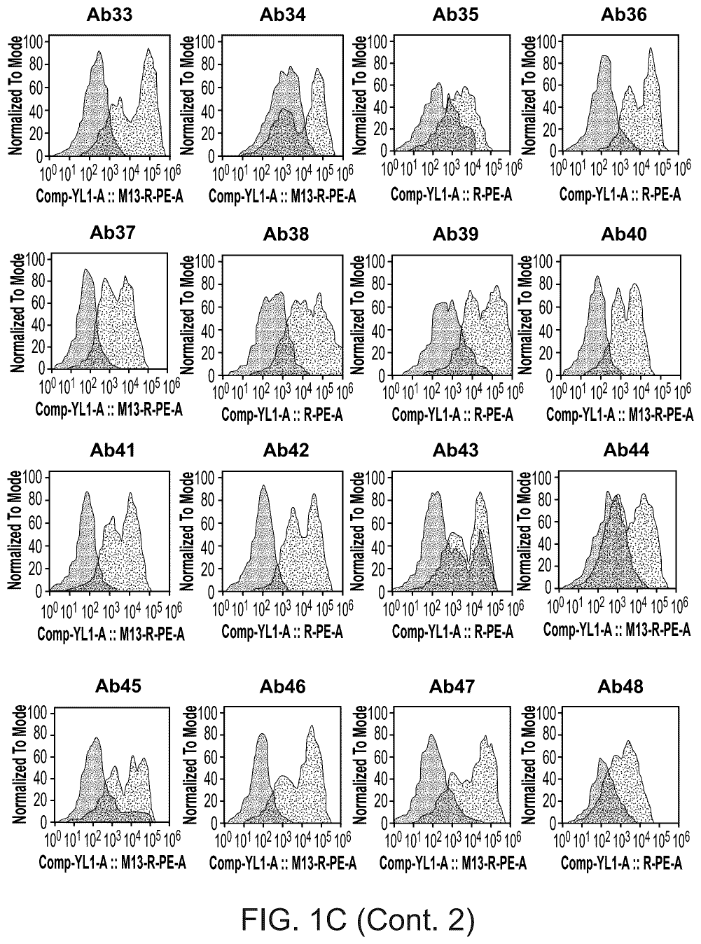

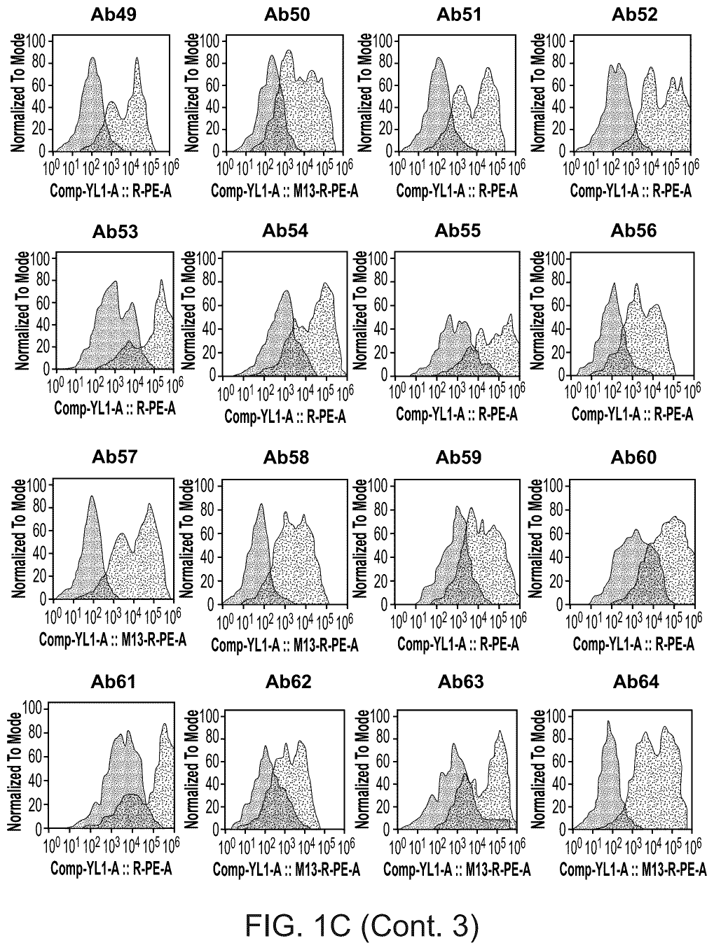

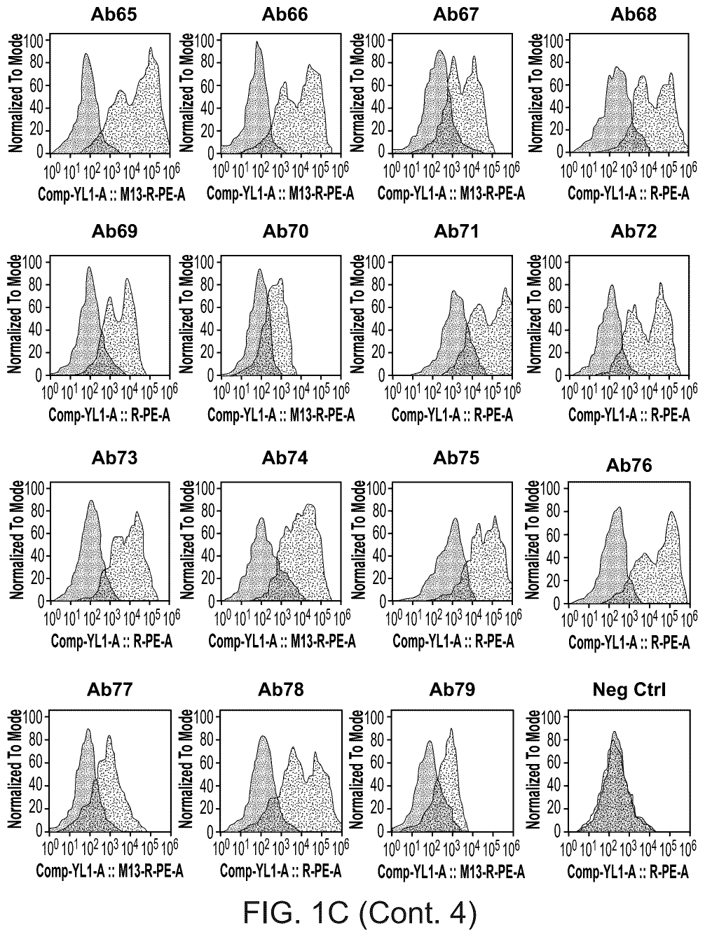

As the polyclonal phage showed antibody enrichment against TMPRSS4, the monoclonal phages were screened for binding to the HEK293T-TMPRSS4 cell line by flow cytometry to identify the positive hits. There were 14 positive hits from round 3 and 303 positive hits from round 4 for a total of 317 positive hits from both rounds of monoclonal screening. The positive hits were analyzed by next-generation sequencing (NGS) to determine the antibody sequences. The sequence results showed 79 unique antibodies. The 79 unique scFv sequences are listed in Table 4. Flow cytometry histograms of the 79 unique antibodies binding to the HEK293T-TMPRSS4 cell line are shown in FIG. 1C.

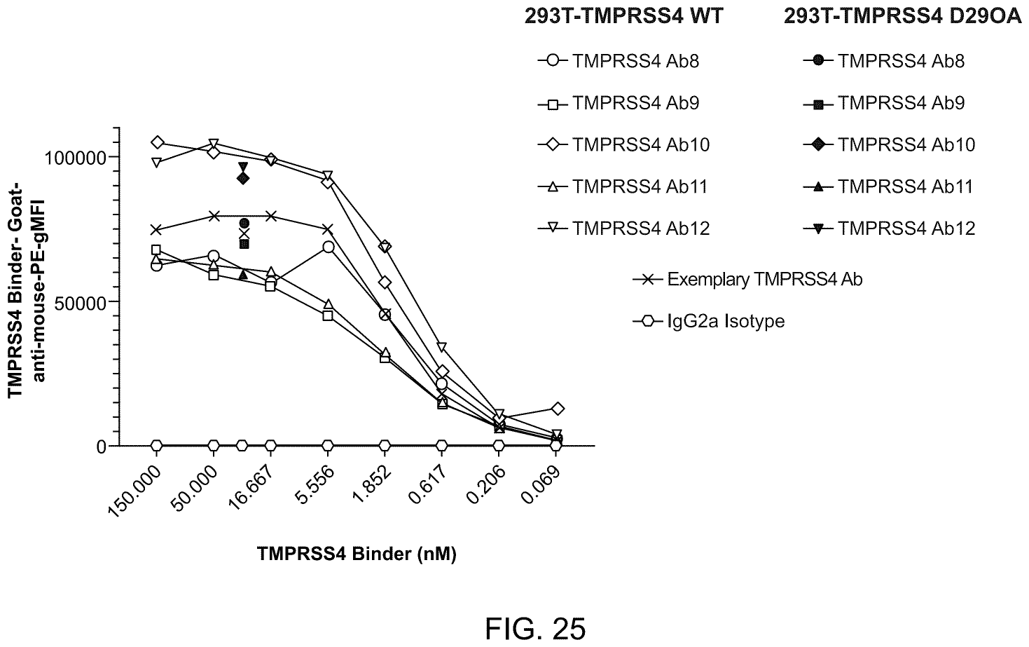

To validate the binding activity of phage antibodies to TMPRSS4, five candidate VH/VL pairs were reformatted to mouse IgG2 chimeric recombinant proteins. Binding of the mIgG2 chimeric recombinant antibodies to TMPRSS4 was assessed by cell-binding dose curves on parental HEK293T and HEK293T-TMPRSS4 cell lines. The five antibodies bound specifically to the TMPRSS4-expressing cell line and did not bind to the TMPRSS4-negative parental cells (FIG. 1A), indicating the antibodies discovered by cell-based phage selection can function as TMPRSS4 antigen recognition domains. In addition, 4 of 5 antibodies were found to bind to the membrane-proximal domain of TMPRSS4 as indicated by binding to both the CL610 cell line expressing the catalytically inactive TMPRSS4 D290A and also to the CL612 cell line expressing the truncated TMPRSS4 that lacks the distal domain of the antigen (FIG. 1B). To validate that binding of the antibodies was independent of catalytic activity, cell binding of the selected antibodies to 293T-TMPRSS4 D290A cells was assessed via flow cytometry. All five TMPRSS4 antibodies tested showed similar binding compared to the 293T-TMPRSS4 WT cells, thereby indicating that the binding was independent of TMPRSS4 catalytic activity (FIG. 25).

### Example 2: Development of TMPRSS4 Constitutive CARs

### Materials and Methods

### Generation of Cells Expressing TMPRSS4 Constitutive CARs

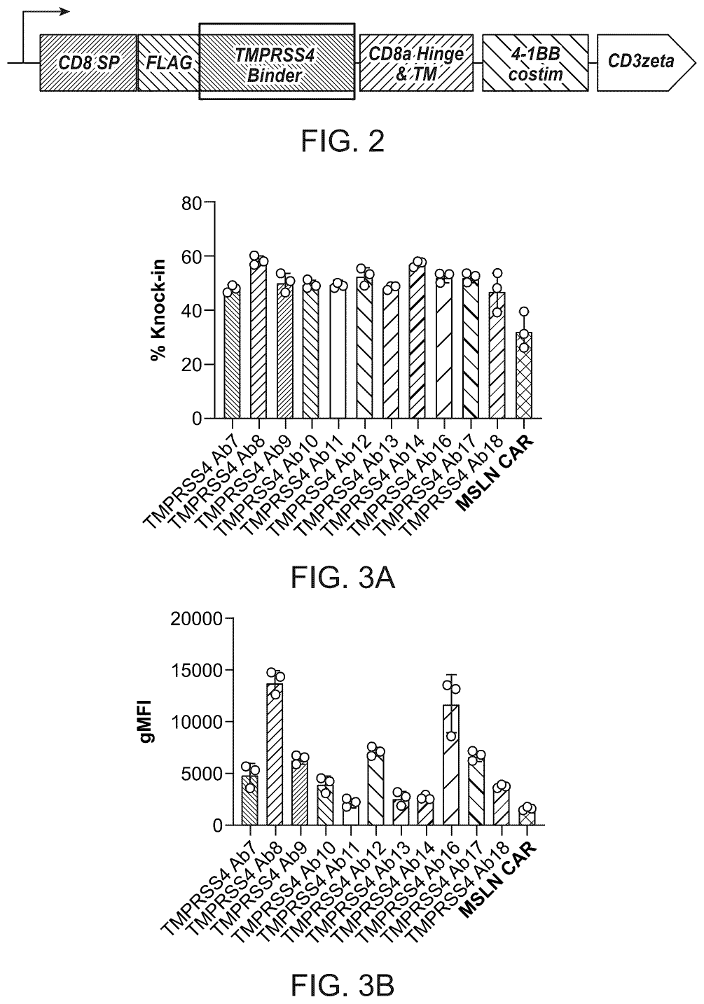

Selected TMPRSS4 antibodies identified in Example 1 and 6 other TMPRSS4 antibodies from the phage screen were next used to prepare chimeric antigen receptors (CARs) for further screening. CAR constructs included (from 5′ to 3′) a CD8 signal peptide (SEQ ID NO: 825), a Flag epitope tag (SEQ ID NO:959), the TMPRSS4-binding scFv, a CD8α hinge domain (SEQ ID NO:821), a CD8α transmembrane domain (SEQ ID NO:822), a 4-1BB costimulatory domain (SEQ ID NO:823), and a CD35 activation domain (SEQ ID NO: 824) (FIG. 2).

T cells were activated for two days using CD3-CD28 beads. At day 2, beads were removed followed by the delivery of the CAR transgene to the GS94 site in the genome of the T cells. Transgene integration was performed using a CRISPR-based process and electroporation step by combining activated T cells, CRISPR/Cas9 RNP with an sgRNA that targeted the GS94 non-coding safe harbor loci integration site, and plasmid DNA constituting a repair template to effect insertion of the transgene cassette via cellular DNA repair machinery.

Following electroporation, cells were recovered and expanded in T cell media for 7 days. Negative control T cells were generated using a mock electroporation process that edited T cells with ribonucleoprotein (RNP) in the absence of donor plasmid (RNP control).

The GS94 CRISPR/Cas9 RNP used was generated by complexing single guide RNA (sgRNA) with recombinant Streptococcus pyogenes Cas9 (SpCas9). The sgRNA contained a protospacer sequence directing the CRISPR/Cas9 RNP to the GS94-transgene integration site. The plasmid DNA repair template contained the CAR transgene cassette, flanked by 450 base pair (bp) sequences homologous to the regions flanking the integration site to effect repair-mediated insertion.

### Flow Cytometry

Cell count and % editing were determined by pelleting cells at 300×g for 5 min, and resuspending in FACS buffer containing anti-FLAG BV421 for surface CAR expression or antibodies for markers of activation (CD25) or exhaustion (TIM3). Following a 20 min staining period at room temperature, cells were spun down and washed 1× with FACS Buffer. Following a spin down, cells were resuspended in 50 μL of FACS buffer, then topped with 50 μL of CountBright Plus counting beads. Data were acquired on an Attune N×T flow cytometer. FSC and SSC parameters were used to specify gates for counting beads versus T cells. Absolute cell count was derived by using the formula: Cells/μL=(Cell count/Counting beads count)×Counting beads concentration from bottle. Fold change of edited T cell number and % edited T cells was determined by the formula: T-cell fold change from D0=(T cell count at D6/T cell count at DO).

### Cytotoxicity Assays

CAR-expressing cells were co-cultured with target cells at varying E:T ratios for 72 hours at 37° C. Following incubation, cytotoxicity was measured using a luciferase reporter assay. Data are presented as the mean±standard deviation of 3 donors.

### Cytokine Secretion

To further assess the specificity and function of T cells expressing CARs, supernatants were collected from target cytotoxicity co-cultures (Effector: Target ratio of 1:1, 72 hour co-culture). Following incubation, supernatants were collected at endpoint and cytokine release levels were measured using a Luminex assay.

### Results

### Generation of Cells Expressing TMPRSS4 Constitutive CARs

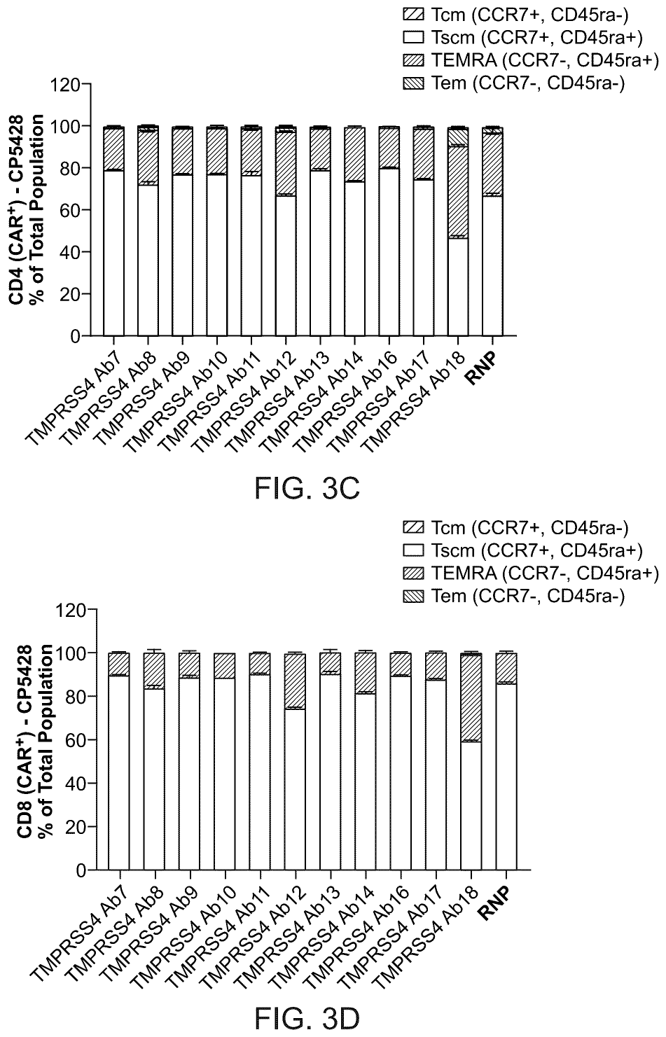

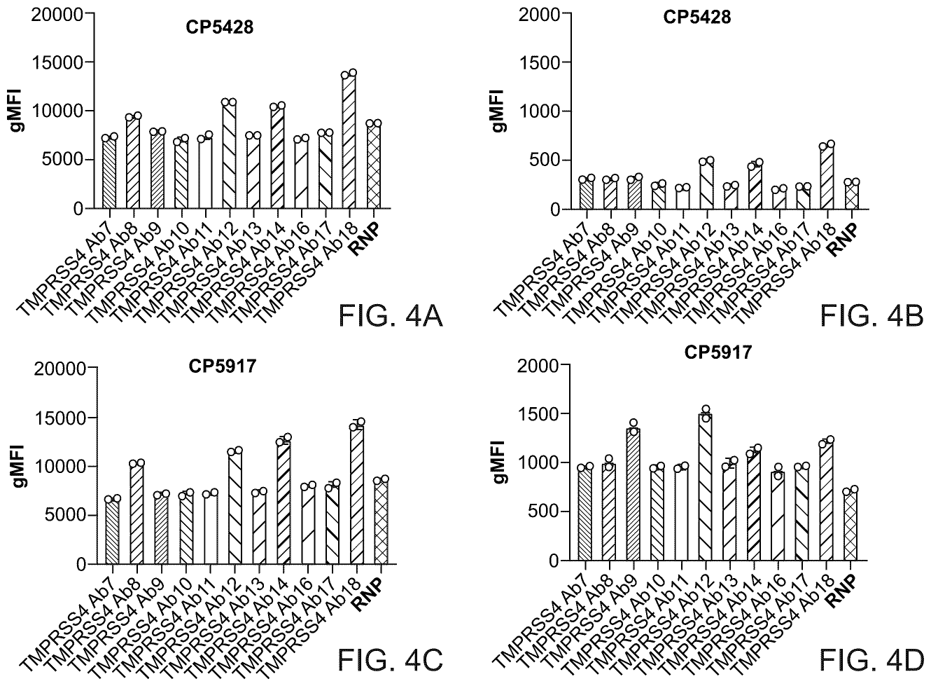

CAR expression constructs were introduced into T cells isolated from three donors by electroporation as described above. CAR expression was measured by flow cytometry to assess the percentage of cells having CAR knock-in (FIG. 3A) and receptor expression level as measured by geometric mean fluorescence intensity (gMFI) (FIG. 3B). CAR-expressing T cells were gated on expression of CD4 or CD8 and further analyzed for expression of CCR7 and CD45RA to classify cells into central memory T cells (T_{CM}; CCR7⁺/CD45RA-), stem cell memory T cells (T_{CSM}; CCR7⁺/CD45RA⁺), effector memory T cells (T_{EM}; CCR7⁻/CD45RA⁻), and effector memory T cells re-expressing CD45RA (T_{EMRA}; CCR7⁻/CD45RA⁺). Similar to RNP control, CAR-expressing cells were predominantly T_{CM} cells with T_{EMRA} cells being the second most prevalent subtype for both CD4⁺ (FIG. 3C) and CD8⁺ (FIG. 3D) T cells. TMPRSS4 CAR-expressing cells also showed similar surface expression of activation marker CD25 (FIGS. 4A and 4C) and exhaustion marker TIM3 (FIGS. 4B and 4D) compared to RNP control.

### Functional Assessment of Cells Expressing TMPRSS4 Constitutive CARs

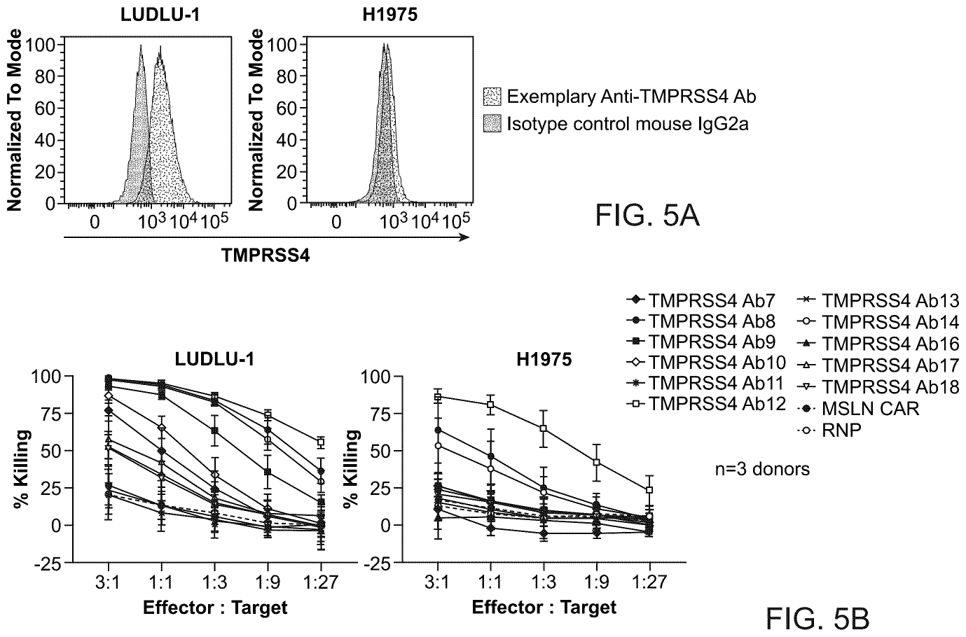

Cells expressing TMPRSS4 CARs were tested for their ability to specifically kill TMPRSS4-expressing target cells. LUDLU-1 lung squamous cell carcinoma cells and H1975 non-small cell lung cancer cells were confirmed to have positive surface expression of TMPRSS4 based on flow cytometry using an exemplary TMPRSS4 antibody (FIG. 5A). T cells expressing a TMPRSS4 CAR, a control CAR, or RNP control were incubated with LUDLU-1 or H1975 target cells for 72 hours at various effector-to-target (E:T) ratios (e.g., 3:1, 1:1, 1:3, 1:9, and 1:27). Percent (%) killing of target cells was measured using a luciferase reporter assay (FIG. 5B). The TMPRSS4 antibodies were ranked based on their ability to induce cytotoxicity of LUDLU-1 and H1975 target cells.

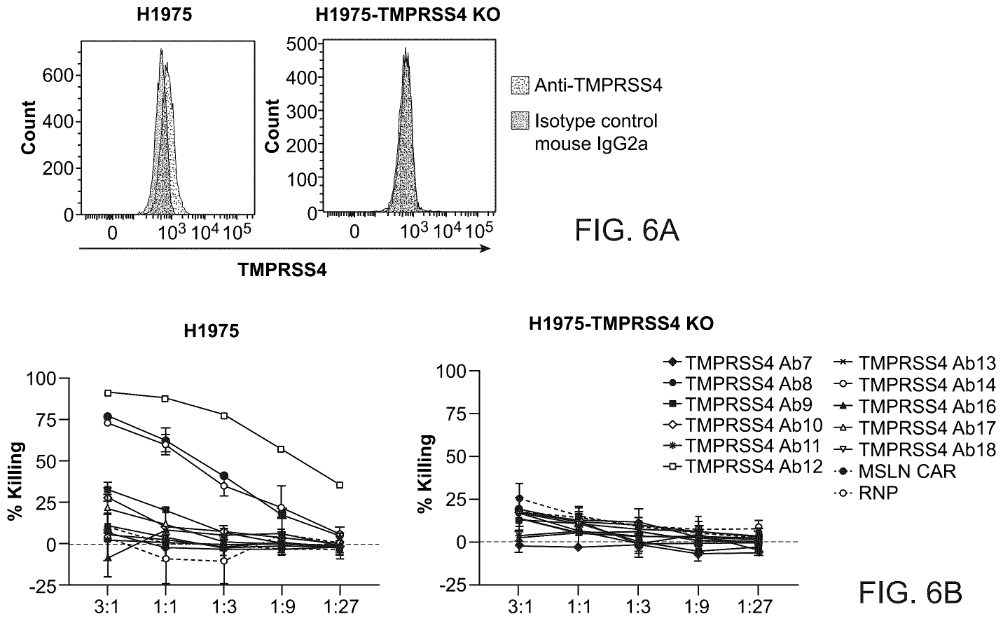

To further assess the specificity of TMPRSS4 CARS, TMPRSS4 was knocked out in H1975 cells, and loss of TMPRSS4 surface expression was confirmed by flow cytometry using the exemplary TMPRSS4 antibody (FIG. 6A). TMPRSS4 CAR-expressing T cells were incubated with wild-type and TMPRSS4-knockout H1975 cells for 72 hours at various E:T ratios, and killing of target cells was measured by luciferase assay. (FIG. 6B). CARs that demonstrated cytotoxicity against wild-type H1975 cells also demonstrated no killing of TMPRSS4-knockout cells, indicating the cytotoxicity induced by the TMPRSS4 CARs was target-specific.

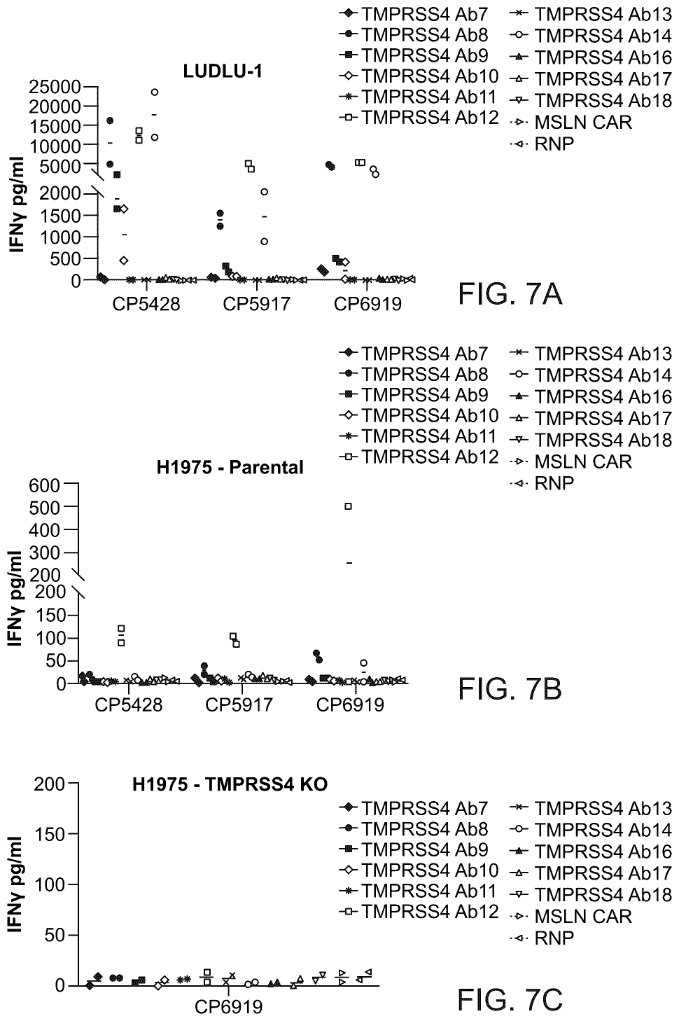

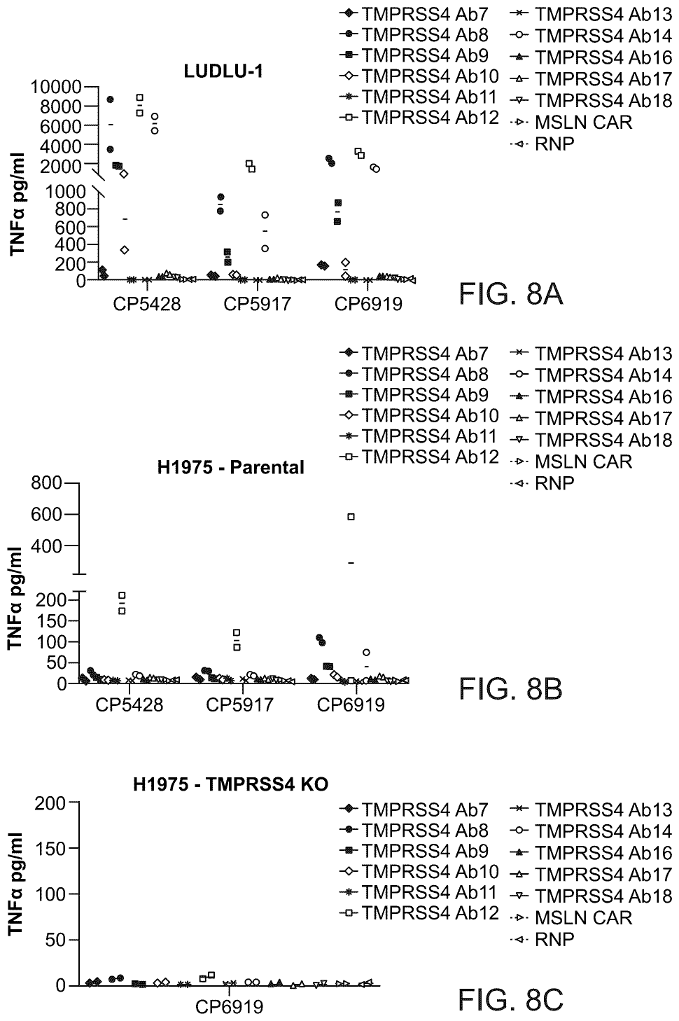

To assess activation of T cells, secretion of interferon gamma (IFNγ) and tumor necrosis factor alpha (TNFα) was measured from T cells incubated for 72 hours with TMPRSS4-positive LUDLU-1 and H1975 target cells, as well as TMPRSS4-knockout H1975 cells at an E:T ratio of 1:1. Secretion of both IFNγ and TNFα was highest in cells incubated with LUDLU-1 cells (FIGS. 7A and 8A), with lower secretion observed from cells incubated wild-type H1975 cells (FIGS. 7B and 8B), and nearly undetectable IFNγ or TNFα secretion was observed from CAR-expressing cells incubated with TMPRSS4 knockout H1975 (H1975-TMPRSS4 KO) cells (FIGS. 7C and 8C). These results align to the levels of TMPRSS4 surface expression observed by flow cytometry for each cell line (FIGS. 5A and 6A).

### Activation of Cells by Different TMPRSS4 Forms

To assess the ability of the TMPRSS4 antibodies to bind to specific forms of TMPRSS4, two mutant constructs of TMPRSS4 were prepared (FIG. 9): one having a mutation ablating serine protease activity (“catalytic inactive” TMPRSS4) and the other having a truncation of the serine protease domain (“cleaved/truncated” TMPRSS4). Wild-type TMPRSS4, as well as the two mutants, were each expressed in HEK293T cells. Transduced HEK293T cells, TMPRSS4-negative parental HEK293T cells, and naturally TMPRSS4-expressing LUDLU-1 cells were incubated with T cells expressing a TMPRSS4 CAR or a control CAR at a 1:1 E:T ratio for 24 hours. T cells were collected after incubation, gated for live cells expressing CD3 and Flag (CAR expression), and analyzed by flow cytometry for expression of early T cell activation marker CD69. Most tested TMPRSS4 antibodies showed similar activation for all three TMPRSS4 forms indicating that they bind to the membrane-proximal domain of TMPRSS4. In contrast, TMPRSS4 Ab 17 showed strong activation by wild-type and catalytic inactive TMPRSS4, but reduced activation by truncated TMPRSS4 (FIG. 10) indicating that it binds to a membrane-distal epitope of TMPRSS4 that is not present in the “cleaved/truncated” antigen format.

### Example 3: Generation and Assessment of SLC34A2 Antibodies

This example provides methods used to generate human anti-Solute Carrier Family 34 Member 2 (SLC34A2) monoclonal antibodies.

Human SLC34A2 (alias: NPT2B) is a multi-pass transmembrane protein of the solute carrier family with annotated 8 transmembrane regions according to Uniprot (entry 095436·NPT2B_HUMAN). Structure predictions identified the longest extracellular loop (herein called ECL2), set forth in SEQ ID NO:1119 and corresponding to residues 234-362 within human SLC34A2 protein, as the optimal target region for raising antibodies.

SLC34A2 extracellular loop
(ECL2; SEQ ID NO: 1119)
VEVATHYLEIITQLIVESFHFKNGEDAPDLLKVITKPFTKLIVQLDKKV
ISQIAMNDEKAKNKSLVKIWCKTFTNKTQINVTVPSTANCTSPSLCWTD
GIQNWTMKNVTYKENIAKCQHIFVNFHLPDL

### A. Immunization Strategies

Human antibodies that bind to SLC34A2 were generated by immunizing mice that were genetically modified to produce antibodies containing fully human antibody variable regions with three different antigen approaches.

The goal of the three different antigen approaches was to generate optimal antibody diversity towards human SLC34A2. Specifically, the mice were immunized with one of the following immunogens:
(i) an SLC34A2-KLH conjugate composed of a SLC34A2 peptide fragment (underlined and bold amino acids in ECL2 sequence, corresponding to amino acids 79-106 of ECL2 sequence set forth in SEQ ID NO: 1119 with a C-terminal Cysteine residue (SEQ ID NO: 1150), which served as an acceptor for conjugation to KLH (Q10583.2; SEQ ID NO: 1151);
(ii) an SLC34A2 recombinant fusion protein (SEQ ID NO: 1152) composed of the human SLC34A2 ECL2 sequence (SEQ ID NO: 1119) fused to a murine Fc domain (SEQ ID NO: 1153);
(iii) cells expressing SLC34A2, namely EpH4 cells, which are a murine breast cancer cell line modified to overexpress full-length human SLC34A2 (SEQ ID NO: 962).

Mice were injected up to 9 times intraperitoneally, subcutaneously, or in the hock. The mice immunized with peptide (i) or cells (iii) were given a priming dose of human SLC34A2 DNA (NM_006424; SEQ ID NO: 963) linked to gold particles that was administered using a gene gun. The spleen and lymph nodes of serum-titer positive mice were harvested and antibodies specific to human SLC34A2 were generated using single B cell cloning.

### B. Single B Cell Cloning and Antibody Sequencing

Single B cell cloning (SBC) was used to isolate SLC34A2-specific monoclonal antibodies from the immunized mice described above. The lymph nodes and/or spleen cells were incubated and stained using markers identifying live and dead cells, markers identifying IgG-positive class-switched memory B-cells, as well as two or more soluble SLC34A2 antigens (SLC34A2 peptide set forth as INVTVPSTANCTSPSLCWTDGIQNWTMK (SEQ ID NO: 1149 and/or SLC34A2 recombinant fusion protein set forth in SEQ ID NO: 1152).

Using a FACS sorter, antigen-specific B cells were individually sorted at 1 cell per well into multi-well plates containing lysis buffer, thereby lysing the cells and releasing the RNA. Lysates were subjected to multiple rounds of PCR to isolate the V_{H} and V_{L} regions of the captured B cell receptor for next generation sequencing (NGS) and to append promoter/signal peptide and constant region blocks to the requisite ends of the variable region.

The final PCR reactions generated Transcriptionally Active PCR (TAP) (Liang et al., J Biol Chem. 2002:277 (5): 3593-8) products that were transfected via high-throughput methods into Expi293 cells and purified using protein A to generate small-scale amounts of recombinant hIgG1/kappa antibody (SEQ ID NOS: 1247 and 1248) material for screening by ELISA and flow cytometry.

The V_{H} and V_{L} regions from the positive human SLC34A2 monoclonal antibodies were recovered and sequenced by next generation sequencing (NGS).

### C. Binding to SLC34A2 Positive Cells

Antibodies were screened by standard flow cytometry methods for binding to human OVCAR-3 human ovarian cancer cells which endogenously express human SLC34A2. HEK293 cells were used as a non-transfected negative control. Briefly, OVCAR-3 and HEK293 cells that had been detached were incubated at 4° C. for 30-60 minutes with 8 serial dilutions of the SLC34A2 antibody (eight 5-fold dilution steps, starting from a top concentration of 133 nM Ab). Cells were washed before adding fluorescently labelled anti-human Fc secondary antibody (AF647 F(ab′)₂ gt-anti-hu IgG Fc-specific; Jackson Cat No 109-606-098) for at least 30 minutes at 4° C. Stained cells were then resuspended in cold FACS buffer and analyzed by flow cytometry for geometric mean fluorescent intensity (GeoMFI) and total cell counts on an iQue automated flow cytometer.

About 262 antibodies that bind specifically to SLC34A2-expressing OVCAR3 cells were identified. 16 antibodies were chosen for further characterization. The sequences of these 16 SLC34A2 antibodies are shown in Table 23 below. Table 23 sets forth the SEQ ID NO corresponding to the sequence for the V_{H} and three HCDRs, and V_{L} and three LCDRs.

TABLE 23
Sequence identifier (SEQ ID NO:) for SLC34A2 Antibodies

| SLC34A2 | SEQ ID NO: | | | | | | | |
|---|---|---|---|---|---|---|---|---|
| Antibody | V_{H} | CDR-H1 | CDR-H2 | CDR-H3 | V_{L} | CDR-L1 | CDR-L2 | CDR-L3 |
| SLC34A2 Ab 1 | 1001 | 1002 | 1003 | 1004 | 1005 | 1006 | 1007 | 1008 |
| SLC34A2 Ab 2 | 1009 | 1010 | 1011 | 1012 | 1013 | 1006 | 1007 | 1014 |
| SLC34A2 Ab 3 | 1015 | 1016 | 1017 | 1018 | 1019/1125 | 1020 | 1021 | 1022 |
| and 3.1 | | | | | | | | |
| SLC34A2 Ab 4 | 1023 | 1024 | 1025 | 1026 | 1027 | 1028 | 1029 | 1030 |
| SLC34A2 Ab 5 | 1031 | 1032 | 1033 | 1034 | 1035/1154 | 1036 | 1037 | 1038 |
| and 5.1 | | | | | | | | |
| SLC34A2 Ab 6 | 1039 | 1040 | 1041 | 1042 | 1043/1155 | 1044 | 1045 | 1046 |
| and 6.1 | | | | | | | | |
| SLC34A2 Ab 7 | 1047 | 1048 | 1049 | 1050 | 1051 | 1036 | 15 | 1052 |
| SLC34A2 Ab 8 | 1053 | 1048 | 1054 | 1055 | 1056 | 1036 | 1057 | 1058 |
| SLC34A2 Ab 9 | 1059 | 1060 | 1061 | 1062 | 1063/1156 | 1064 | 1021 | 1065 |
| and 9.1 | | | | | | | | |
| SLC34A2 Ab 10 | 1066 | 1067 | 1068 | 1069 | 1070/1233 | 1028 | 1071 | 1072 |
| and 10.1 | | | | | | | | |
| SLC34A2 Ab 11 | 1073 | 1032 | 1074 | 1075 | 1076 | 1251 | 15 | 1077 |
| SLC34A2 Ab 12 | 1078 | 1079 | 1080 | 1081 | 1082 | 1036 | 15 | 1083 |
| SLC34A2 Ab 13 | 1084 | 1085 | 1086 | 1087 | 1088 | 1089 | 1021 | 1065 |
| SLC34A2 Ab 14 | 1090 | 1010 | 1091 | 1092 | 1093 | 1006 | 1007 | 1008 |
| SLC34A2 Ab 15 | 1094 | 1095 | 1003 | 1096 | 1097/1234 | 1036 | 1098 | 1099 |
| and 15.1 | | | | | | | | |
| SLC34A2 Ab 16 | 1100 | 1101 | 1102 | 1096 | 1103/1250 | 1251 | 1104 | 1105 |
| and 16.1 | | | | | | | | |

### Example 4: SLC34A2 Antibody Epitope Binding

This example provides methods used to determine the epitope recognized by SLC34A2 monoclonal antibodies in Table 21 of Example 3.

The epitope recognized by the SLC34A2 antibodies was determined by ELISA binding assays using the SLC34A2 peptide immunogen (SEQ ID NO: 1149), the ECL-mFc fusion protein (SEQ ID NO: 1249), and a biotinylated irrelevant peptide as negative control. Briefly, NeutrAvidin plates were coated at 1 μg/ml overnight, washed and incubated with blocking buffer for at least 1 hr. Biotinylated antigens as well as irrelevant antigens were then added at 1 μg/ml for at least 1 hr at RT, followed by rigorous washing. HRP-conjugated anti-human Fc detection reagent (goat anti-human IgG-Fc-HRP cat: 109-036-098; Jackson ImmunoResearch) was applied and incubated at 4° C. for 30-60 min. After additional wash steps, 3,3′,5,5′-Tetramethylbenzidine (TMB) substrate was added and optical density read using an appropriate plate reader.

The epitope identified for each of the 16 antibodies is shown in Table 24. EC50 values for binding SLC34A2-expressing OVCAR3 cells as determined in Example 3 are also shown in

TABLE 24
Several strong antibodies with an EC50 less than 15 nM were identified.
Table 24. EC50 for SLC34A2 Antibodies

| | | | | | Max | |
|---|---|---|---|---|---|---|
| | | | | SEQ | Signal by | EC50 [nM] |
| SLC34A2 | | Epitope | | ID | flow | by flow |
| Antibody | Immunogen | Recognized | HCDR3 | NO: | cytometry | cytometry |
| Ab 8 | mECL2-Fc | N.N. | HPAGYSSSWSAFDI | 1055 | 354 | 6 |
| | protein | | | | | |
| Ab 11 | mECL2-Fc | N.N. | HPRGIAARWGNWFDP | 1075 | 224 | 6 |
| | protein | | | | | |
| Ab 4 | DNA + cells | ECL2+/peptide- | DGPLWGNYFDY | 1026 | 277 | 5 |
| Ab 5 | DNA + cells | ECL2+/peptide- | HGRGTIGYFDY | 1034 | 271 | 5 |
| Ab 6 | DNA + cells | ECL2+/peptide- | QGTNWGLYFDY | 1042 | 211 | 6 |
| Ab 7 | mECL2-Fc | ECL2+/peptide | HPAGYSTRWSAFDI | 1050 | 278 | 16 |
| | protein | weak | | | | |
| Ab 12 | mECL2-Fc | peptide 1+/ | HPRGSYGANFDY | 1081 | 305 | 5 |
| | protein | ECL2+ | | | | |
| Ab 13 | mECL2-Fc | peptide 1+/ | IPALRFLEWLP | 1087 | 233 | 41 |
| | protein | ECL2+ | | | | |
| Ab 3 | mECL2-Fc | peptide 1+/ | IPVLRFLEWLP | 1018 | 232 | 24 |
| | protein | ECL2+ | | | | |
| Ab 9 | mECL2-Fc | peptide 1+/ | IPVSRFLEWLP | 1062 | 281 | 37 |
| | protein | ECL2+ | | | | |
| Ab 10 | mECL2-Fc | peptide 1+/ | RGYTYGYFFDY | 1069 | 218 | 20 |
| | protein | ECL2+ | | | | |
| Ab 1 | Peptide-1 | peptide 1+/ | WMTKVKGYFDY | 1004 | 353 | 7 |
| | | ECL2+ | | | | |
| Ab 2 | Peptide-1 | peptide 1+/ | WMTTIKGYFDY | 1012 | 330 | 7 |
| | | ECL2+ | | | | |
| Ab 11 | Peptide-1 | peptide 1+/ | WMTTVKGYFDY | 1092 | 288 | 9 |
| | | ECL2+ | | | | |
| Ab 15 | Peptide-1 | peptide 1+/ | YIVGRPGFNWFDP | 1096 | 346 | 8 |
| | | ECL2+ | | | | |
| Ab 16 | Peptide-1 | peptide 1+/ | YIVGRPGFNWFDP | 1096 | 296 | 6 |
| | | ECL2+ | | | | |

N.N. means epitope is unknown; ECL2 corresponds to SEQ ID NO: 1119; peptide 1 corresponds to SEQ ID NO: 1149.

### Example 5: Development of TMPRSS4 CAR and SLC34A2 Priming Receptor Logic Gates

### Materials and Methods

### Logic Gate Construction and Screening

A library of 368 circuits (also called logic gates), composed of 8 TMPRSS4 CARs from an initial 78 CAR library, and 17 SLC34A2 PrimeRs, with some tested in two scFv orientations (e.g., V_{H}-V_{L} and V_{L}-V_{H}) was cloned with and without fidelity tuning and tested to identify circuits with maximized potency and fidelity profiles. The antibody combinations used in the 368 circuits is provided in Table 25.

TABLE 25
Circuit Library

| Logic | | |
|---|---|---|
| Gate | TMPRSS4 | SCL34A2 |
| (LG) # | scFv | scFv |
| 1 | AB 14 | Ab 4 |
| 2 | AB 40 | Ab 4 |
| 3 | AB 48 | Ab 4 |
| 4 | AB 62 | Ab 4 |
| 5 | AB 12 | Ab 4 |
| 6 | AB 7 | Ab 4 |
| 7 | AB 49 | Ab 4 |
| 8 | AB 29 | Ab 4 |
| 9 | AB 14 | Ab 6 |
| 10 | AB 40 | Ab 6 |
| 11 | AB 48 | Ab 6 |
| 12 | AB 62 | Ab 6 |
| 13 | AB 12 | Ab 6 |
| 14 | AB 7 | Ab 6 |
| 15 | AB 49 | Ab 6 |
| 16 | AB 29 | Ab 6 |
| 17 | AB 14 | Ab 12 |
| 18 | AB 40 | Ab 12 |
| 19 | AB 48 | Ab 12 |
| 20 | AB 62 | Ab 12 |
| 21 | AB 12 | Ab 12 |
| 22 | AB 7 | Ab 12 |
| 23 | AB 49 | Ab 12 |
| 24 | AB 29 | Ab 12 |
| 25 | AB 14 | Ab 3 |
| 26 | AB 40 | Ab 3 |
| 27 | AB 48 | Ab 3 |
| 28 | AB 62 | Ab 3 |
| 29 | AB 12 | Ab 3 |
| 30 | AB 7 | Ab 3 |
| 31 | AB 49 | Ab 3 |
| 32 | AB 29 | Ab 3 |
| 33 | AB 14 | Ab 1 |
| 34 | AB 40 | Ab 1 |
| 35 | AB 48 | Ab 1 |
| 36 | AB 62 | Ab 1 |
| 37 | AB 12 | Ab 1 |
| 38 | AB 7 | Ab 1 |
| 39 | AB 49 | Ab 1 |
| 40 | AB 29 | Ab 1 |
| 41 | AB 14 | Ab 2 |
| 42 | AB 40 | Ab 2 |
| 43 | AB 48 | Ab 2 |
| 44 | AB 62 | Ab 2 |
| 45 | AB 12 | Ab 2 |
| 46 | AB 7 | Ab 2 |
| 47 | AB 49 | Ab 2 |
| 48 | AB 29 | Ab 2 |
| 49 | AB 14 | Ab 16 |
| 50 | AB 40 | Ab 16 |
| 51 | AB 48 | Ab 16 |
| 52 | AB 62 | Ab 16 |
| 53 | AB 12 | Ab 16 |
| 54 | AB 7 | Ab 16 |
| 55 | AB 49 | Ab 16 |
| 56 | AB 29 | Ab 16 |
| 57 | AB 14 | Ab 8 |
| 58 | AB 40 | Ab 8 |
| 59 | AB 48 | Ab 8 |
| 60 | AB 62 | Ab 8 |
| 61 | AB 12 | Ab 8 |
| 62 | AB 7 | Ab 8 |
| 63 | AB 49 | Ab 8 |
| 64 | AB 29 | Ab 8 |
| 65 | AB 14 | Ab 5 |
| 66 | AB 40 | Ab 5 |
| 67 | AB 48 | Ab 5 |
| 68 | AB 62 | Ab 5 |
| 69 | AB 12 | Ab 5 |
| 70 | AB 7 | Ab 5 |
| 71 | AB 49 | Ab 5 |
| 72 | AB 29 | Ab 5 |
| 73 | AB 14 | Ab 7 |
| 74 | AB 40 | Ab 7 |
| 75 | AB 48 | Ab 7 |
| 76 | AB 62 | Ab 7 |
| 77 | AB 12 | Ab 7 |
| 78 | AB 7 | Ab 7 |
| 79 | AB 49 | Ab 7 |
| 80 | AB 29 | Ab 7 |
| 81 | AB 14 | Ab 13 |
| 82 | AB 40 | Ab 13 |
| 83 | AB 48 | Ab 13 |
| 84 | AB 62 | Ab 13 |
| 85 | AB 12 | Ab 13 |
| 86 | AB 7 | Ab 13 |
| 87 | AB 49 | Ab 13 |
| 88 | AB 29 | Ab 13 |
| 89 | AB 14 | Ab 10 |
| 90 | AB 40 | Ab 10 |
| 91 | AB 48 | Ab 10 |
| 92 | AB 62 | Ab 10 |
| 93 | AB 12 | Ab 10 |
| 94 | AB 7 | Ab 10 |
| 95 | AB 49 | Ab 10 |
| 96 | AB 29 | Ab 10 |
| 97 | AB 14 | Ab 14 |
| 98 | AB 40 | Ab 14 |
| 99 | AB 48 | Ab 14 |
| 100 | AB 62 | Ab 14 |
| 101 | AB 12 | Ab 14 |
| 102 | AB 7 | Ab 14 |
| 103 | AB 49 | Ab 14 |
| 104 | AB 29 | Ab 14 |
| 105 | AB 14 | Ab 15 |
| 106 | AB 40 | Ab 15 |
| 107 | AB 48 | Ab 15 |
| 108 | AB 62 | Ab 15 |
| 109 | AB 12 | Ab 15 |
| 110 | AB 7 | Ab 15 |
| 111 | AB 49 | Ab 15 |
| 112 | AB 29 | Ab 15 |
| 113 | AB 14 | Ab 4 |
| 114 | AB 40 | Ab 4 |
| 115 | AB 48 | Ab 4 |
| 116 | AB 62 | Ab 4 |
| 117 | AB 12 | Ab 4 |
| 118 | AB 7 | Ab 4 |
| 119 | AB 49 | Ab 4 |
| 120 | AB 29 | Ab 4 |
| 121 | AB 14 | Ab 6 |
| 122 | AB 40 | Ab 6 |
| 123 | AB 48 | Ab 6 |
| 124 | AB 62 | Ab 6 |
| 125 | AB 12 | Ab 6 |
| 126 | AB 7 | Ab 6 |
| 127 | AB 49 | Ab 6 |
| 128 | AB 29 | Ab 6 |
| 129 | AB 14 | Ab 12 |
| 130 | AB 40 | Ab 12 |
| 131 | AB 48 | Ab 12 |
| 132 | AB 62 | Ab 12 |
| 133 | AB 12 | Ab 12 |
| 134 | AB 7 | Ab 12 |
| 135 | AB 49 | Ab 12 |
| 136 | AB 29 | Ab 12 |
| 137 | AB 14 | Ab 3 |
| 138 | AB 40 | Ab 3 |
| 139 | AB 48 | Ab 3 |
| 140 | AB 62 | Ab 3 |
| 141 | AB 12 | Ab 3 |
| 142 | AB 7 | Ab 3 |
| 143 | AB 49 | Ab 3 |
| 144 | AB 29 | Ab 3 |
| 145 | AB 14 | Ab 1 |
| 146 | AB 40 | Ab 1 |
| 147 | AB 48 | Ab 1 |
| 148 | AB 62 | Ab 1 |
| 149 | AB 12 | Ab 1 |
| 150 | AB 7 | Ab 1 |
| 151 | AB 49 | Ab 1 |
| 152 | AB 29 | Ab 1 |
| 153 | AB 14 | Ab 2 |
| 154 | AB 40 | Ab 2 |
| 155 | AB 48 | Ab 2 |
| 156 | AB 62 | Ab 2 |
| 157 | AB 12 | Ab 2 |
| 158 | AB 7 | Ab 2 |
| 159 | AB 49 | Ab 2 |
| 160 | AB 29 | Ab 2 |
| 161 | AB 14 | Ab 16 |
| 162 | AB 40 | Ab 16 |
| 163 | AB 48 | Ab 16 |
| 164 | AB 62 | Ab 16 |
| 165 | AB 12 | Ab 16 |
| 166 | AB 7 | Ab 16 |
| 167 | AB 49 | Ab 16 |
| 168 | AB 29 | Ab 16 |
| 169 | AB 14 | Ab 9 |
| 170 | AB 40 | Ab 9 |
| 171 | AB 48 | Ab 9 |
| 172 | AB 62 | Ab 9 |
| 173 | AB 12 | Ab 9 |
| 174 | AB 7 | Ab 9 |
| 175 | AB 49 | Ab 9 |
| 176 | AB 29 | Ab 9 |
| 177 | AB 14 | Ab 11 |
| 178 | AB 40 | Ab 11 |
| 179 | AB 48 | Ab 11 |
| 180 | AB 62 | Ab 11 |
| 181 | AB 12 | Ab 11 |
| 182 | AB 7 | Ab 11 |
| 183 | AB 49 | Ab 11 |
| 184 | AB 29 | Ab 11 |
| 185 | AB 14 | Ab 17 |
| 186 | AB 40 | Ab 17 |
| 187 | AB 48 | Ab 17 |
| 188 | AB 62 | Ab 17 |
| 189 | AB 12 | Ab 17 |
| 190 | AB 7 | Ab 17 |
| 191 | AB 49 | Ab 17 |
| 192 | AB 29 | Ab 17 |
| 193 | AB 14 | Ab 4 |
| 194 | AB 40 | Ab 4 |
| 195 | AB 48 | Ab 4 |
| 196 | AB 62 | Ab 4 |
| 197 | AB 12 | Ab 4 |
| 198 | AB 7 | Ab 4 |
| 199 | AB 49 | Ab 4 |
| 200 | AB 29 | Ab 4 |
| 201 | AB 14 | Ab 6 |
| 202 | AB 40 | Ab 6 |
| 203 | AB 48 | Ab 6 |
| 204 | AB 62 | Ab 6 |
| 205 | AB 12 | Ab 6 |
| 206 | AB 7 | Ab 6 |
| 207 | AB 49 | Ab 6 |
| 208 | AB 29 | Ab 6 |
| 209 | AB 14 | Ab 12 |
| 210 | AB 40 | Ab 12 |
| 211 | AB 48 | Ab 12 |
| 212 | AB 62 | Ab 12 |
| 213 | AB 12 | Ab 12 |
| 214 | AB 7 | Ab 12 |
| 215 | AB 49 | Ab 12 |
| 216 | AB 29 | Ab 12 |
| 217 | AB 14 | Ab 3 |
| 218 | AB 40 | Ab 3 |
| 219 | AB 48 | Ab 3 |
| 220 | AB 62 | Ab 3 |
| 221 | AB 12 | Ab 3 |
| 222 | AB 7 | Ab 3 |
| 223 | AB 49 | Ab 3 |
| 224 | AB 29 | Ab 3 |
| 225 | AB 14 | Ab 1 |
| 226 | AB 40 | Ab 1 |
| 227 | AB 48 | Ab 1 |
| 228 | AB 62 | Ab 1 |
| 229 | AB 12 | Ab 1 |
| 230 | AB 7 | Ab 1 |
| 231 | AB 49 | Ab 1 |
| 232 | AB 29 | Ab 1 |
| 233 | AB 14 | Ab 2 |
| 234 | AB 40 | Ab 2 |
| 235 | AB 48 | Ab 2 |
| 236 | AB 62 | Ab 2 |
| 237 | AB 12 | Ab 2 |
| 238 | AB 7 | Ab 2 |
| 239 | AB 49 | Ab 2 |
| 240 | AB 29 | Ab 2 |
| 241 | AB 14 | Ab 16 |
| 242 | AB 40 | Ab 16 |
| 243 | AB 48 | Ab 16 |
| 244 | AB 62 | Ab 16 |
| 245 | AB 12 | Ab 16 |
| 246 | AB 7 | Ab 16 |
| 247 | AB 49 | Ab 16 |
| 248 | AB 29 | Ab 16 |
| 249 | AB 14 | Ab 8 |
| 250 | AB 40 | Ab 8 |
| 251 | AB 48 | Ab 8 |
| 252 | AB 62 | Ab 8 |
| 253 | AB 12 | Ab 8 |
| 254 | AB 7 | Ab 8 |
| 255 | AB 49 | Ab 8 |
| 256 | AB 29 | Ab 8 |
| 257 | AB 14 | Ab 5 |
| 258 | AB 40 | Ab 5 |
| 259 | AB 48 | Ab 5 |
| 260 | AB 62 | Ab 5 |
| 261 | AB 12 | Ab 5 |
| 262 | AB 7 | Ab 5 |
| 263 | AB 49 | Ab 5 |
| 264 | AB 29 | Ab 5 |
| 265 | AB 14 | Ab 7 |
| 266 | AB 40 | Ab 7 |
| 267 | AB 48 | Ab 7 |
| 268 | AB 62 | Ab 7 |
| 269 | AB 12 | Ab 7 |
| 270 | AB 7 | Ab 7 |
| 271 | AB 49 | Ab 7 |
| 272 | AB 29 | Ab 7 |
| 273 | AB 14 | Ab 13 |
| 274 | AB 40 | Ab 13 |
| 275 | AB 48 | Ab 13 |
| 276 | AB 62 | Ab 13 |
| 277 | AB 12 | Ab 13 |
| 278 | AB 7 | Ab 13 |
| 279 | AB 49 | Ab 13 |
| 280 | AB 29 | Ab 13 |
| 281 | AB 14 | Ab 10 |
| 282 | AB 40 | Ab 10 |
| 283 | AB 48 | Ab 10 |
| 284 | AB 62 | Ab 10 |
| 285 | AB 12 | Ab 10 |
| 286 | AB 7 | Ab 10 |
| 287 | AB 49 | Ab 10 |
| 288 | AB 29 | Ab 10 |
| 289 | AB 14 | Ab 14 |
| 290 | AB 40 | Ab 14 |
| 291 | AB 48 | Ab 14 |
| 292 | AB 62 | Ab 14 |
| 293 | AB 12 | Ab 14 |
| 294 | AB 7 | Ab 14 |
| 295 | AB 49 | Ab 14 |
| 296 | AB 29 | Ab 14 |
| 297 | AB 14 | Ab 15 |
| 298 | AB 40 | Ab 15 |
| 299 | AB 48 | Ab 15 |
| 300 | AB 62 | Ab 15 |
| 301 | AB 12 | Ab 15 |
| 302 | AB 7 | Ab 15 |
| 303 | AB 49 | Ab 15 |
| 304 | AB 29 | Ab 15 |
| 305 | AB 14 | Ab 4 |
| 306 | AB 40 | Ab 4 |
| 307 | AB 48 | Ab 4 |
| 308 | AB 62 | Ab 4 |
| 309 | AB 12 | Ab 4 |
| 310 | AB 7 | Ab 4 |
| 311 | AB 49 | Ab 4 |
| 312 | AB 29 | Ab 4 |
| 313 | AB 14 | Ab 6 |
| 314 | AB 40 | Ab 6 |
| 315 | AB 48 | Ab 6 |
| 316 | AB 62 | Ab 6 |
| 317 | AB 12 | Ab 6 |
| 318 | AB 7 | Ab 6 |
| 319 | AB 49 | Ab 6 |
| 320 | AB 29 | Ab 6 |
| 321 | AB 14 | Ab 12 |
| 322 | AB 40 | Ab 12 |
| 323 | AB 48 | Ab 12 |
| 324 | AB 62 | Ab 12 |
| 325 | AB 12 | Ab 12 |
| 326 | AB 7 | Ab 12 |
| 327 | AB 49 | Ab 12 |
| 328 | AB 29 | Ab 12 |
| 329 | AB 14 | Ab 3 |
| 330 | AB 40 | Ab 3 |
| 331 | AB 48 | Ab 3 |
| 332 | AB 62 | Ab 3 |
| 333 | AB 12 | Ab 3 |
| 334 | AB 7 | Ab 3 |
| 335 | AB 49 | Ab 3 |
| 336 | AB 29 | Ab 3 |
| 337 | AB 14 | Ab 1 |
| 338 | AB 40 | Ab 1 |
| 339 | AB 48 | Ab 1 |
| 340 | AB 62 | Ab 1 |
| 341 | AB 12 | Ab 1 |
| 342 | AB 7 | Ab 1 |
| 343 | AB 49 | Ab 1 |
| 344 | AB 29 | Ab 1 |
| 345 | AB 14 | Ab 2 |
| 346 | AB 40 | Ab 2 |
| 347 | AB 48 | Ab 2 |
| 348 | AB 62 | Ab 2 |
| 349 | AB 12 | Ab 2 |
| 350 | AB 7 | Ab 2 |
| 351 | AB 49 | Ab 2 |
| 352 | AB 29 | Ab 2 |
| 353 | AB 14 | Ab 16 |
| 354 | AB 40 | Ab 16 |
| 355 | AB 48 | Ab 16 |
| 356 | AB 62 | Ab 16 |
| 357 | AB 12 | Ab 16 |
| 358 | AB 7 | Ab 16 |
| 359 | AB 49 | Ab 16 |
| 360 | AB 29 | Ab 16 |
| 361 | AB 14 | Ab 17 |
| 362 | AB 40 | Ab 17 |
| 363 | AB 48 | Ab 17 |
| 364 | AB 62 | Ab 17 |
| 365 | AB 12 | Ab 17 |
| 366 | AB 7 | Ab 17 |
| 367 | AB 49 | Ab 17 |
| 368 | AB 29 | Ab 17 |

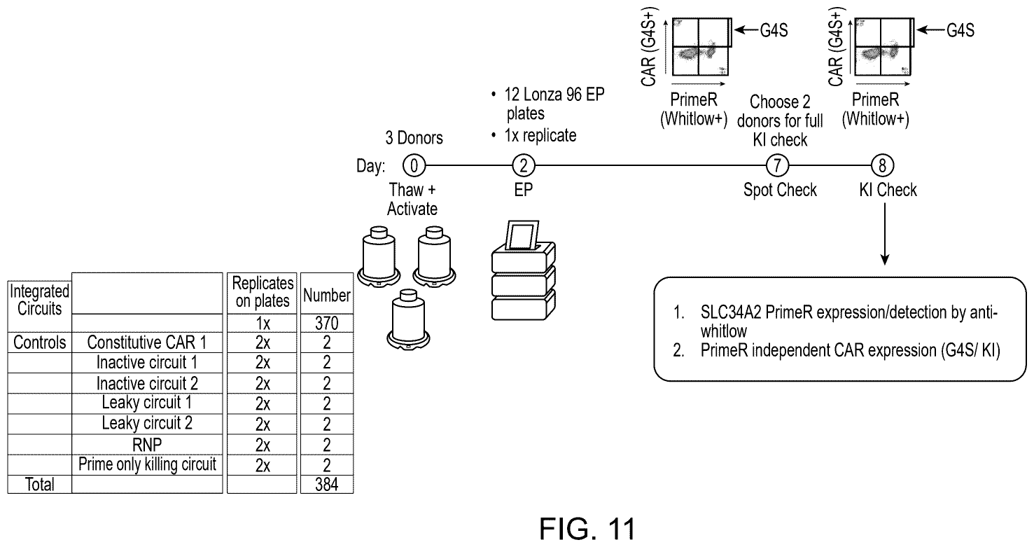

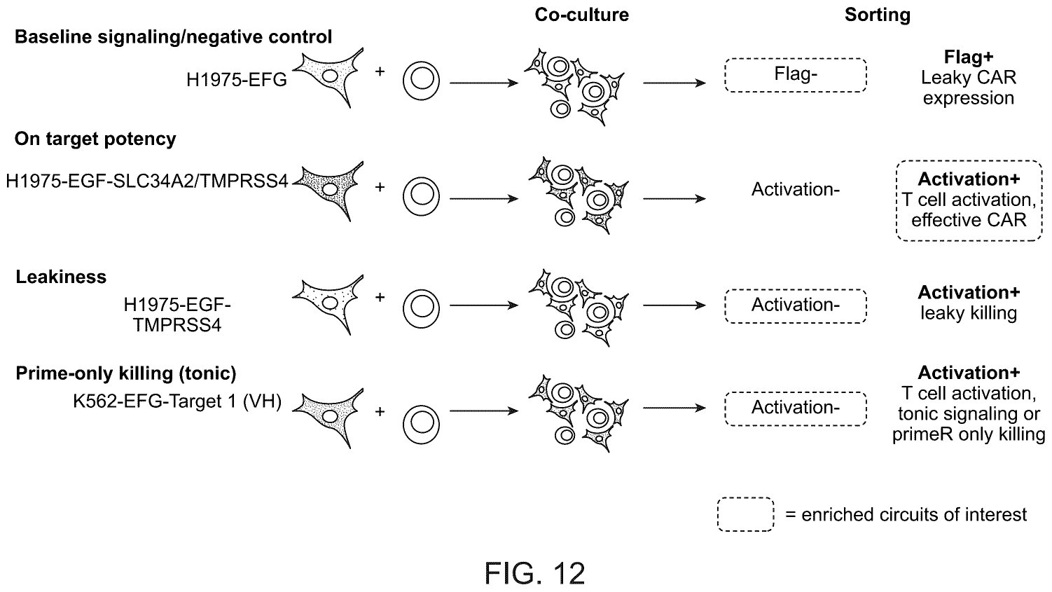

Arrayed testing and down selection occurred in 2 steps. First, all circuits (i.e., logic gates) were engineered into T-cells from 2 donors and the expression of CARs and PrimeRs was evaluated by flow cytometry. A subset of 86 circuits (e.g., logic gates) was selected and these were engineered into T cells from two more donors followed by functional testing in co-culture assays. Finally, the top 5 circuits were selected for in vitro and in vivo pre clinical testing with deep functional assessment. A schematic of the T cell engineering process is provided in FIG. 11. A schematic for assessing T cells expressing a logic gate comprising a an SLC34A2 primeR and a TMPRSS4 CAR is provided in FIG. 12.

### T-Cell Engineering

T cells were activated for two days using CD3-CD28 beads. At day 2, beads were removed followed by the delivery of the CAR transgene to the GS94 site in the genome of the T cells. Transgene integration was performed using a CRISPR-based process and electroporation step by combining activated T cells, CRISPR/Cas9 RNP with an sgRNA that targeted the GS94 non-coding safe harbor loci integration site, and plasmid DNA constituting a repair template to effect insertion of the transgene cassette via cellular DNA repair machinery.

Following electroporation, cells were recovered and expanded in T cell media for 7 days. Negative control T cells were generated using a mock electroporation process that edited T cells with ribonucleoprotein (RNP) in the absence of donor plasmid (RNP control).

### T-Cell Characterization by Flow Cytometry

Cell count and % editing were determined by pelleting cells at 300×g for 5 min, and resuspending in FACS buffer containing anti-G4S-AF647 for surface CAR expression and anti-Whitlow-PE for the surface expression of primeR (% knock in (KI)). Following a 1 hr staining period at 4° C., cells were spun down and washed 1× with FACS Buffer. Following a spin down, cells were resuspended in 50 μL of FACS buffer, then topped with 50 μL of CountBright Plus counting beads. Data were acquired on an Attune NXT flow cytometer. FSC and SSC parameters were used to specify gates for counting beads versus T cells. Absolute cell count was derived by using the formula: Cells/μL=(Cell count/Counting beads count)×Counting beads concentration from bottle.

Co-Culture with Tumor Lines and Luc Based Cytotoxicity

Engineered cells were co-cultured with target cells (H1975-TMPRSS4 D290A) at varying E:T ratios for 72 hours at 37° C. Following incubation, cytotoxicity was measured using a luciferase reporter assay. Data are presented as the mean±standard deviation.

### Cytokine Secretion by Lumit

To further assess the specificity and function of T cells expressing CARs, supernatants were collected from target cytotoxicity co-cultures. Following incubation, supernatants were collected at endpoint and cytokine release levels were measured using a Lumit ELISA assay according to the manufacturer protocol.

### Repetitive Stimulation Assay (RSA)

Engineered T-cells were co-cultured with dual-antigen expressing tumor cells (SLC34A2-TMPRSS4) which also express GFP, at an E:T of 1:9 (one ICT for every 9 tumor cells) at 37° C. Every 3 days, the supernatant containing the T-cells was collected and half of it was cocultured with fresh tumor cells (volumetric split), for up to 10 days. Tumor cell viability was tracked continuously using the Incucyte imaging system. Data represents area under the curve±standard deviation.

### Results

A library composed of 8 TMPRSS4 CARs paired with 17 SLC34A2 PrimeRs in V_{L}-V_{H} orientation and 7 SLC34A2 primeRs in V_{H}-V_{L} orientation, was cloned into logic gates. The majority of primeR and CAR combinations were cloned with and without a Synthetic poly A modification on the primeR gene (2x_syn_pA), to increase potential fidelity of the circuits. The resulting library had 368 circuits in total. These were engineered in arrayed format into naive T-cells to generate logic gated (LG) T-cells (also called integrated circuit T cells or ICTs).

To select the optimal circuits, which demonstrated antigen sensitivity, high potency against tumor cells, and high logic gate fidelity, the following characteristics were evaluated in the LG cells:
% Knock-in (KI) and primeR MFI
Basal leaky expression of CAR
T-cell activity (Cytotoxicity and cytokine secretion) upon co culture with tumor cells

Down selection was performed in two tiers. In the first tier, all 368 LG cells and relevant controls were engineered into 2 donor T-cells. % KI and basal CAR expression were measured using flow cytometry via detection of Whitlow-linker in the primeR and G4S linker in the CAR genes. Leaky CAR conversion was calculated by % CAR/% KI. Circuits with >7.5% conversion were excluded from further consideration. Additionally, any LG samples with <7% KI were also excluded. Analysis was performed separately for each donor and any circuits that were inconsistent between the resulting candidate lists were excluded as well.

Finally, a small number of circuits were excluded or included based on overall performance and additional data from prior studies (rational inclusion or exclusion).

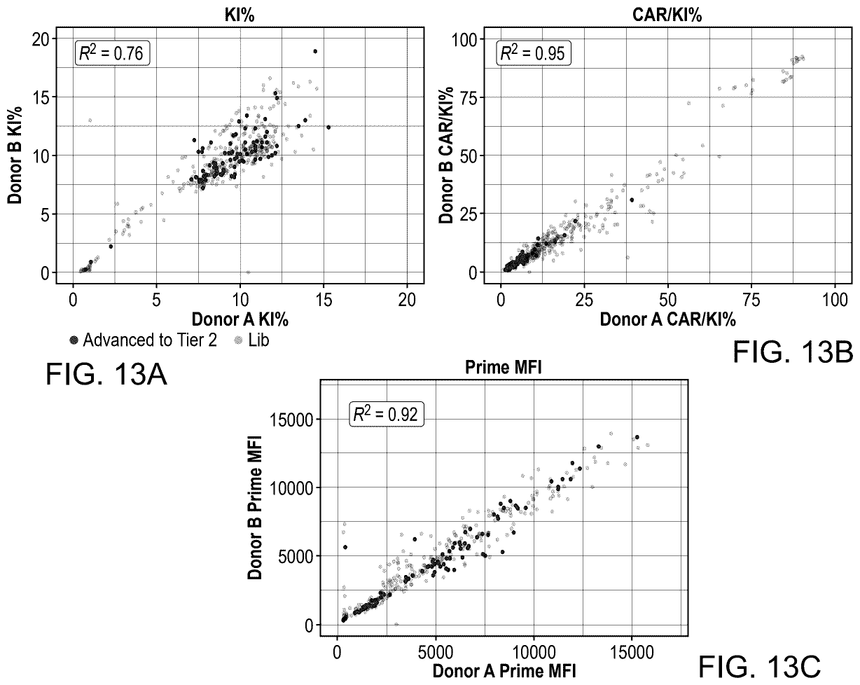

This down-selection step resulted in a list of 86 circuits with acceptable behavior for % gene knock in (KI) and % conversion (FIGS. 13A and 13B) and a range of primeR expression levels (FIG. 13C). The dark spots indicate logic gates (or circuits) that advanced to Tier 2 selection.

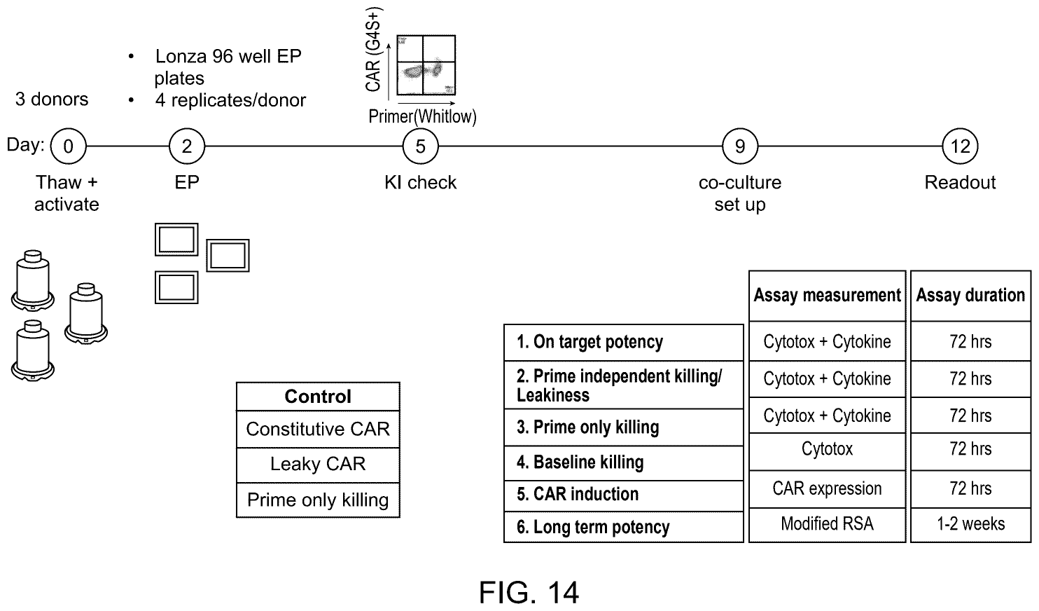

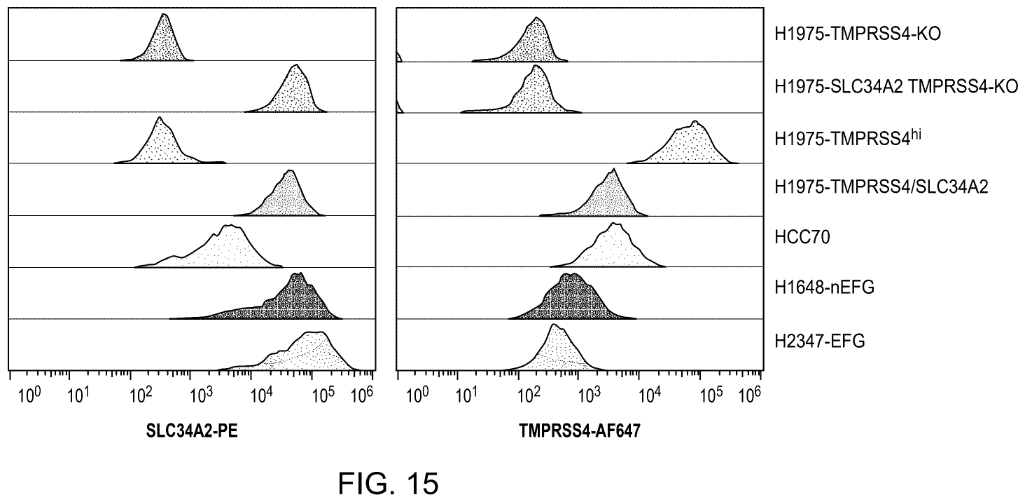

In tier 2, the selected subset of 86 circuits were engineered into 2 additional donor T-cells. % KI was evaluated again, and the cells were co-cultured with several tumor cell lines at various E:Ts (a schematic of the process is provided in FIG. 14). To evaluate potency, 72 hour co-cultures were done with three tumor lines; H1975 NSCLC line engineered with both TMPRSS4 and SLC34A2 at levels that match patient tumor samples (HCC70 was used as surrogate to show relevant TMPRSS4 antigen levels), and two endogenous antigen expression lines—H1648, H2347 which express slightly lower levels of TMPRSS4 compared to the engineered line (FIG. 15). Additionally, the engineered dual antigen H1975 line was used in a repetitive stimulation assay (RSA), to test long term activity of the cells. To measure circuit fidelity, H1975 cells expressing only TMPRSS4 or SLC34A2 were used. Basal activity was demonstrated using H1975 with no expression of either antigen (FIG. 15).

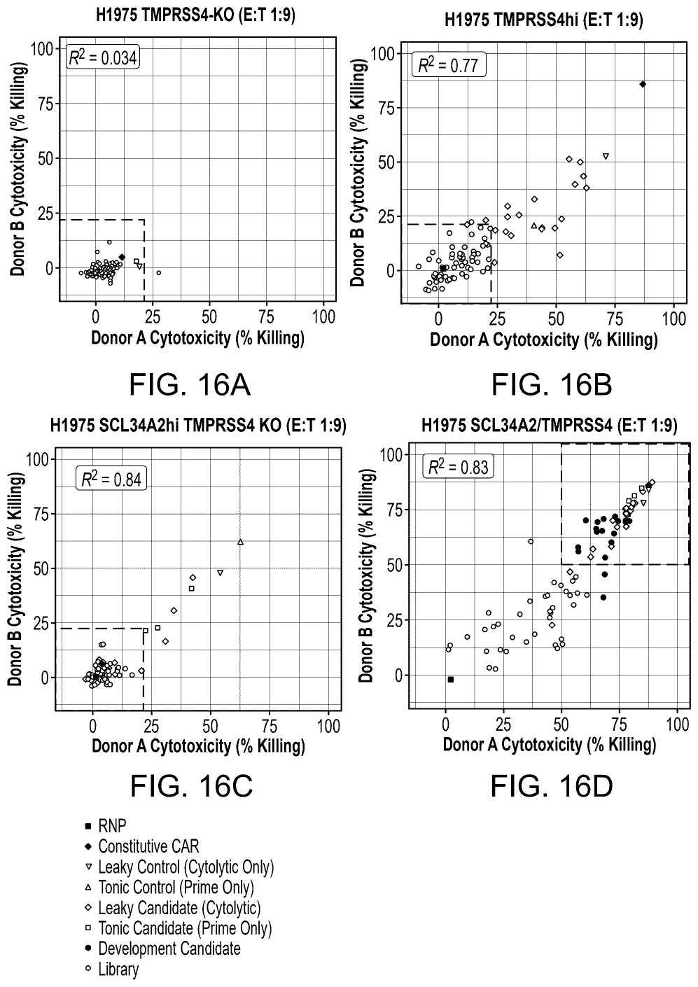

T-cells activity was evaluated by measuring % cytotoxicity at the end of each 72 h co-culture. RNP cells were used for normalizing basal differences between responses to the various tumor lines. For selection of top 23 LG candidates, H1975 based co-cultures with a matched E:T of 1:9 were used. The variance in the basal activity co-culture was calculated and used to create a gate of 3 standard deviations (3xSD, 22.5%) above RNP for each co culture. Any circuits demonstrating activity outside of this gate in the fidelity co-cultures, were excluded (FIGS. 16A, 16B, and 16C). FIG. 16A shows the % killing (cytotoxicity) by engineered logic gate T cells from Donor B compared to the % killing (cytotoxicity) by engineered logic gate T cells from Donor A of TMPRSS4 knockout (KO) cells. 16B shows the % killing (cytotoxicity) by engineered logic gate T cells from Donor B compared to the % killing (cytotoxicity) by engineered logic gate T cells from Donor A of TMPRSS4hi (high TMPRSS4 expression) cells. 16C shows the % killing (cytotoxicity) by engineered logic gate T cells from Donor B compared to the % killing (cytotoxicity) by engineered logic gate T cells from Donor A of SLC34A2hi (high SLC34A2 expression)-TMPRSS4 knockout (KO) cells. 16D shows the % killing (cytotoxicity) by engineered logic gate T cells from Donor B compared to the % killing (cytotoxicity) by engineered logic gate T cells from Donor A of SLC34A2-TMPRSS4 expressing cells. Additionally, any LG samples with <50% on target cytotoxicity were also excluded. This strategy yielded a list of top 23 LG candidates (FIG. 16D).

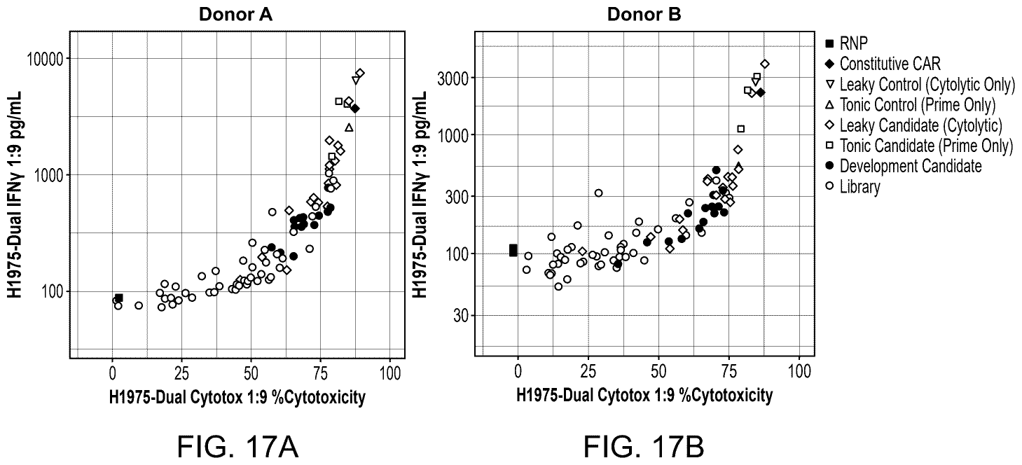

For further characterization supernatants were collected from co-cultures of T cells and SLC34A2-TMPRSS4 expressing cells at E:T of 1:9 and IFNγ secretion was measured by ELISA. Cytokine secretion was well correlated with cytotoxicity for both donors (FIGS. 17A and 17B).

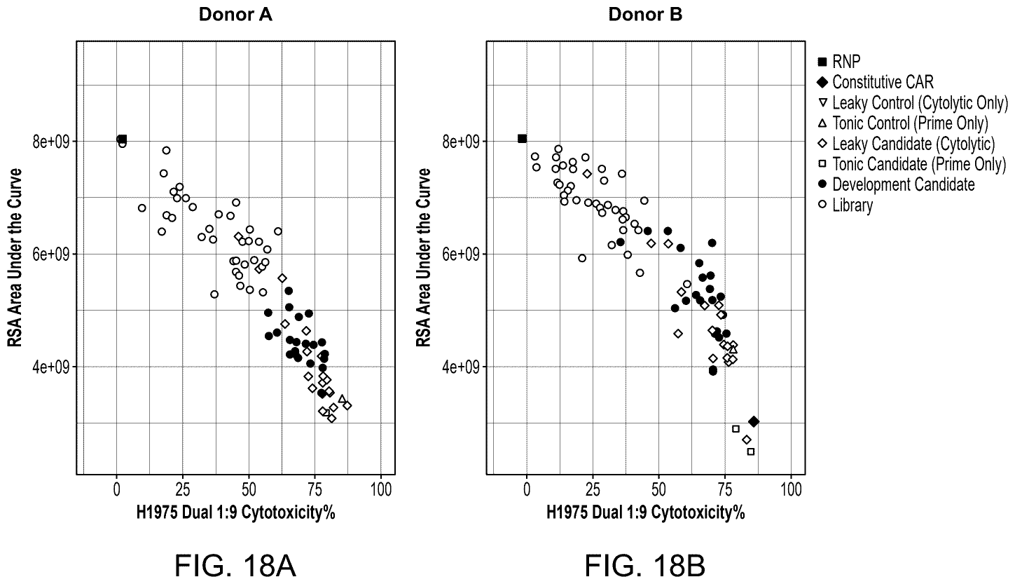

Additionally, the results from the RSA, in which tumor cells viability was assessed, were inversely correlated with the cytotoxicity results of SLC34A2-TMPRSS4 expressing cells, indicating that LG cells were ranked similarly in both acute (72 h) and long term (RSA) potency assays (FIGS. 18A and 18B).

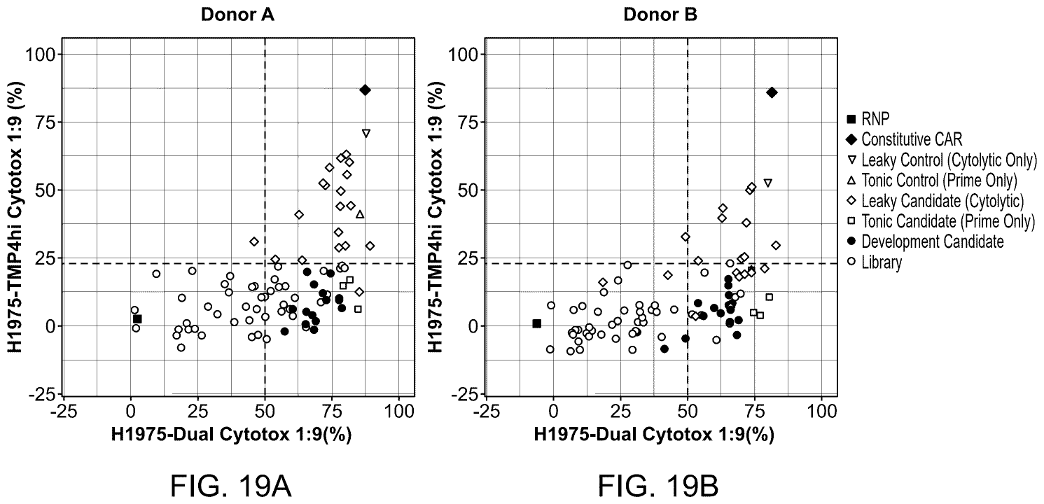

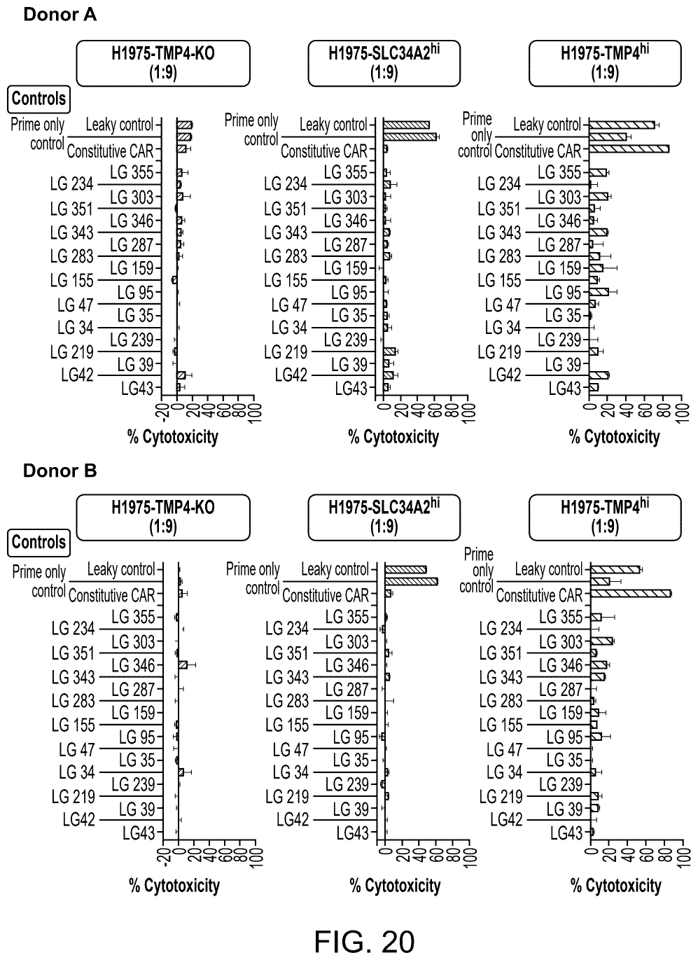

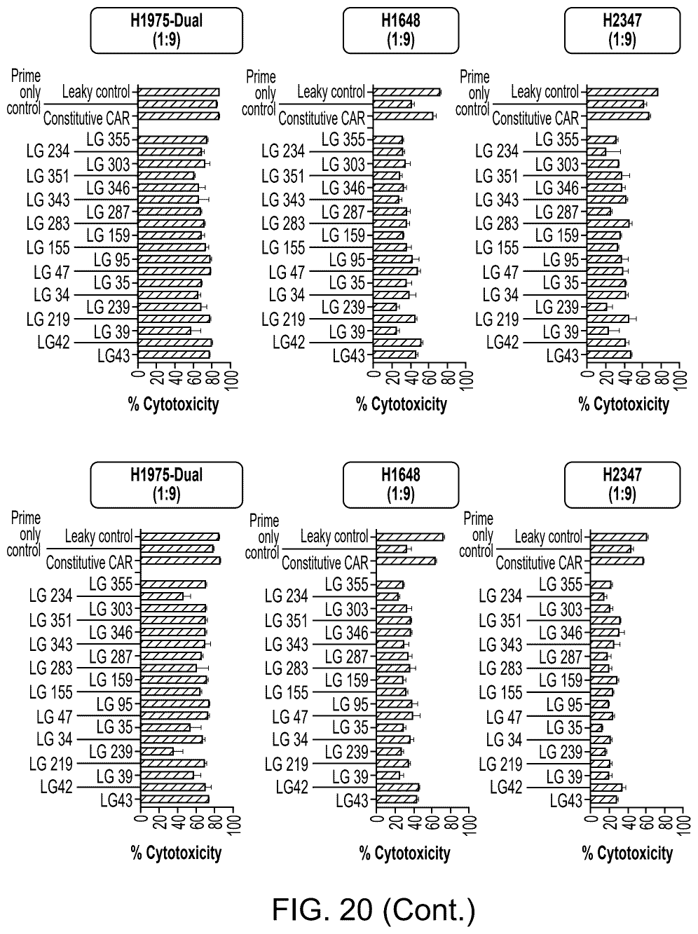

The top 23 candidates show the desired activity profile of high potency and high fidelity (low killing of cytolytic only target line) compared to the rest of the library, while maintaining a small range of variation in their individual profiles. All 23 candidates demonstrate potency even when co-cultured with tumor lines expressing lower levels of TMPRSS4 and SLC34A2 (FIGS. 19A and 19B and FIG. 20).

In order to select the best 5 LG circuits of these top 23, ranking was generated which combined measurements from all of the collected cytotoxicity data in all tested E:Ts, including the RSA. For this ranking the Z-score for each data point in each assay was calculated. The z-scores were averaged for each LG circuit across the two categories of activity. For on-target potency, Z-scores were averaged for all 3 dual-antigen expressing lines, along with the RSA scores. For fidelity, z-scores for the single antigen expressing lines were averaged. Thus two scores for each LG circuit-one for potency and one for fidelity were generated.

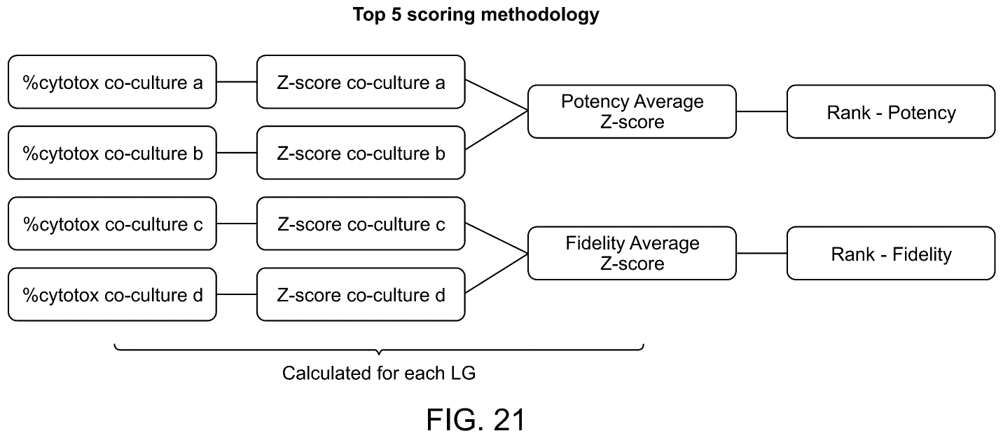

The scores were used to rank the circuits separately for fidelity and for potency and then selected the best in each category (two prioritizing fidelity and three prioritizing potency), which were consistent between the two donors tested (FIG. 21).

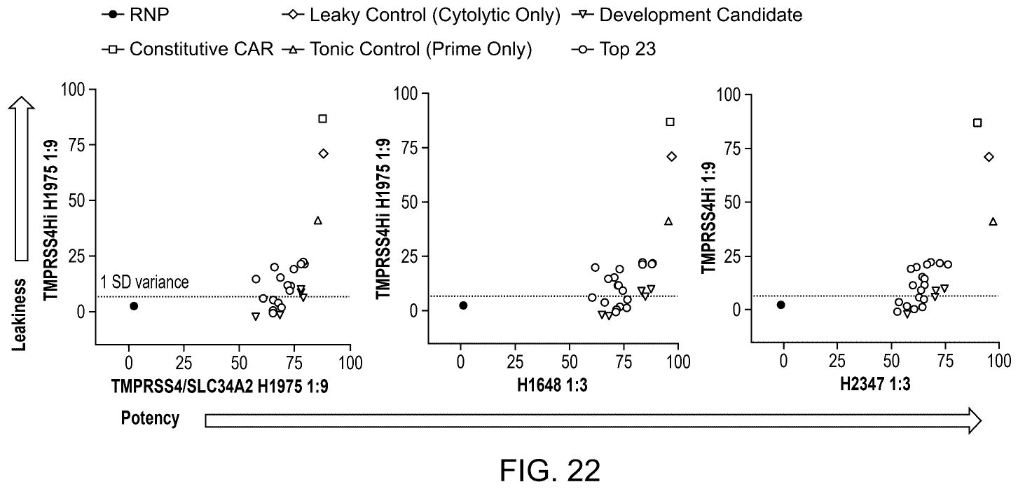

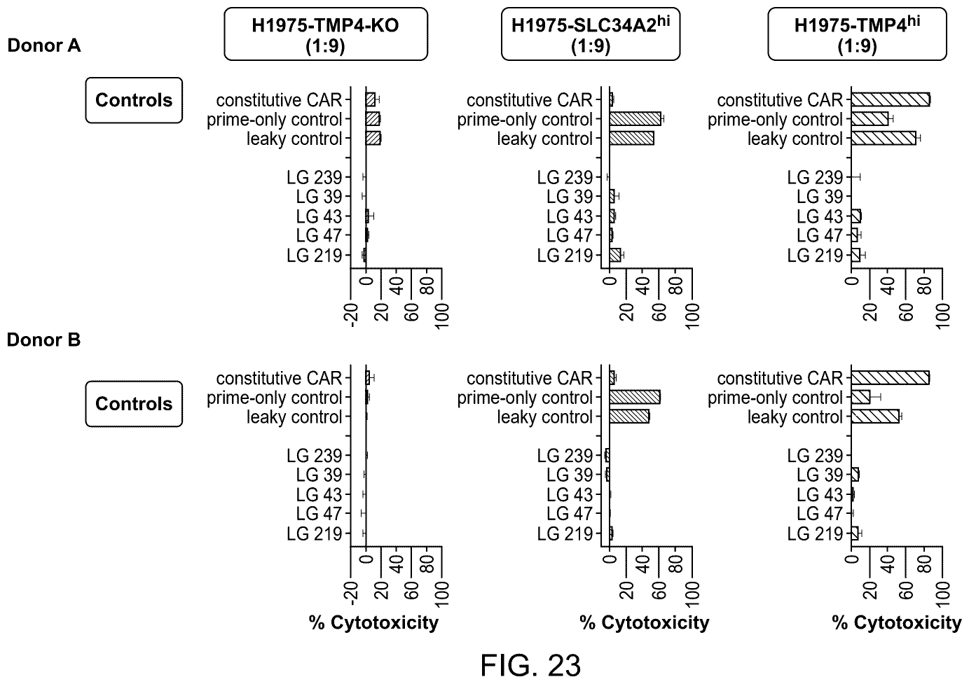

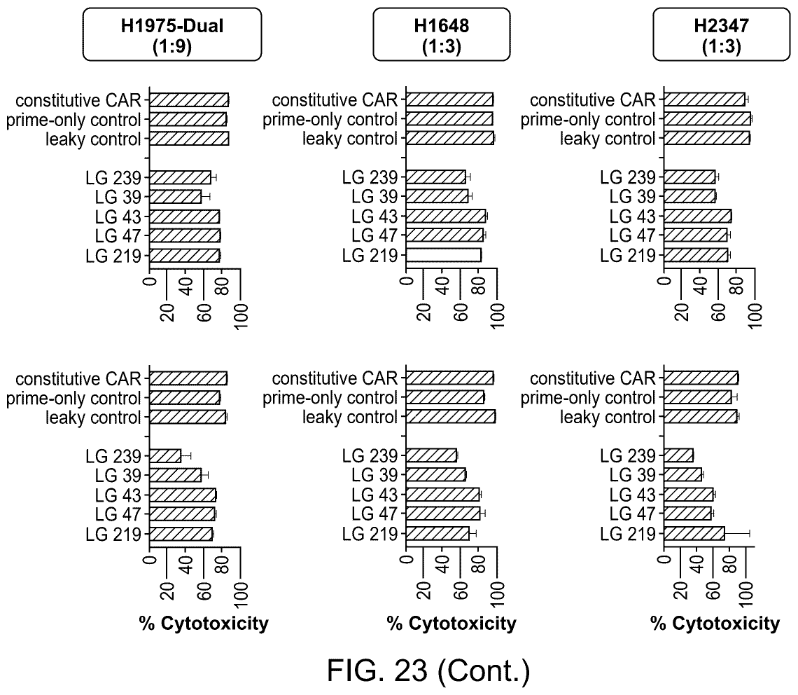

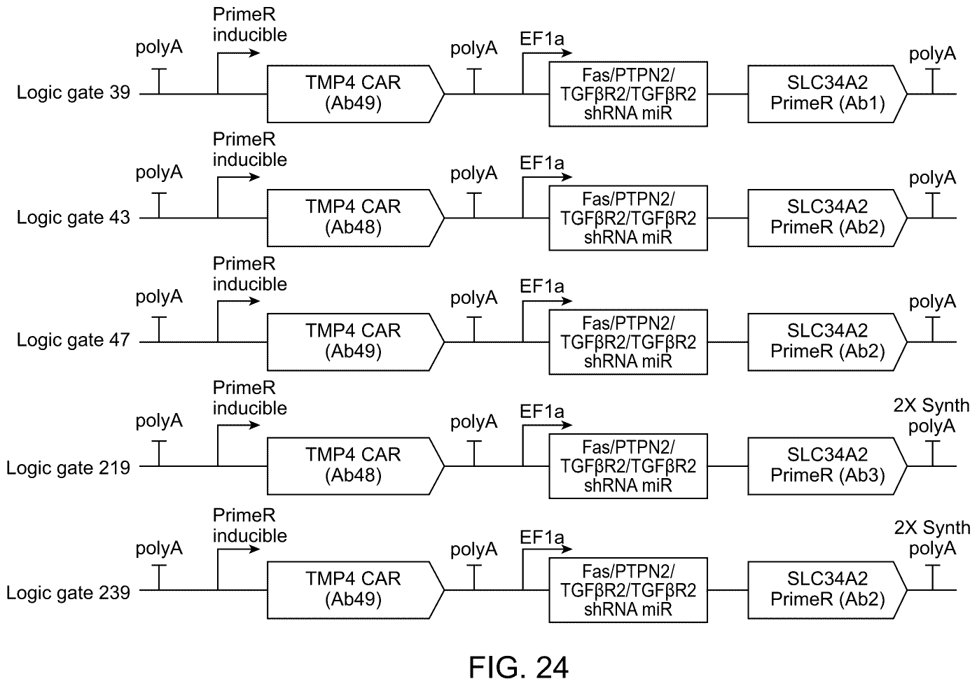

The top five candidates selected using this methodology exhibit the desired high fidelity and high potency profile for logic gate activity. All five candidates maintain potency when co-cultured with tumor lines expressing lower levels of TMPRSS4 and SLC34A2, and show little to no measurable activity when co-cultured with target cells expressing only TMPRSS4 or only SLC34A2 (FIG. 22 and FIG. 23). The five candidates were LG 239, LG 39, LG 43, LG 219, and LG 47. The DNA sequences of the logic gate circuit inserts are provided in SEQ ID NOs: 1120-1124. The LG 239 circuit comprises a synthetic polyA signal (2×polyA, SEQ ID NO: 993) while the LG 47 circuit comprises a bGH poly A signal (SEQ ID NO: 995). Table 26 provides the TMPRSS4 and SLC34A2 V_{H} and V_{L} sequences used in the scFv's used in the five circuits. Diagrams of the logic gate circuits are provided in FIG. 24.

TABLE 26

| | TMPRSS4 scFv in CAR | | | SLC34A2 scFv in primeR | | |
|---|---|---|---|---|---|---|
| | TMPRSS4 | VH SEQ | VL SEQ | SLC34A2 | VH SEQ | VL SEQ |
| LG # | Ab # | ID NO | ID NO | Ab # | ID NO | ID NO |
| LG 239 | Ab 49 | 326 | 327 | Ab 2 | 1009 | 1013 |
| (SEQ ID NO: 1121) | | | | | | |
| LG 39 | Ab 49 | 326 | 327 | Ab 1 | 1001 | 1005 |
| (SEQ ID NO: 1122) | | | | | | |
| LG 43 | Ab 48 | 319 | 320 | Ab 2 | 1009 | 1013 |
| (SEQ ID NO: 1123) | | | | | | |
| LG 219 | Ab 48 | 319 | 320 | Ab 3 | 1015 | 1125 |
| (SEQ ID NO: 1120) | | | | | | |
| LG 47 | Ab 49 | 326 | 327 | Ab 2 | 1009 | 1013 |
| (SEQ ID NO: 1124) | | | | | | |

### Example 6: In Vitro Characterization of TMPRSS4 CAR and SLC34A2 Priming Receptor Logic Gate T Cells

### Materials and Methods

### FAS and TGFBR2 Expression Via Flow Cytometry

Reduction of FAS and TGFBR2 in T cells engineered to express an shRNA module targeting different genes alone or in combination was determined via flow cytometry by staining with anti-CD95 (FAS) FITC antibody and an anti-TGFβ receptor II PE antibody and analyzed on an Attune N×T Flow Cytometer. Expression in transgene positive T cells (e.g., T cells comprising the PrimeR/CAR and shRNA module insert) was compared to transgene negative T cells (e.g., T cells without the PrimeR/CAR and shRNA module insert) to generate a total percentage of reduction. % Reduction (flow) was calculated using the formula: 1-(CD95/TGFBR2 gMFI of PrimeR+/CD95/TGFBR2 gMFI of PrimeR−)*100. Data are presented as the mean±standard deviation averaged across 4 donors.

### PTPN2 Expression Via Western Blot Analysis

Engineered logic gate (LG) T cells expressing an shRNA module targeting different genes alone or in combination were enriched via magnetic-bead based column enrichment to obtain a pure transgene-positive (PrimeR/CAR and shRNA module insert positive) fraction using an anti-Whitlow linker Alexa Fluor 647 antibody and anti-Cy5/anti-alexa fluor 647 MicroBeads. A pure transgene-negative (PrimeR/CAR and shRNA module insert negative) fraction was also obtained by depleting any transgene-positive cells from using the same magnetic-bead based column enrichment. Both populations of edited T cells were then lysed and heated to reduce and denature proteins. Total protein was quantified using the Pierce BCA Protein Assay Kit. Normalized lysates were then loaded into an SDS-PAGE gel and run. Protein was transferred from the gel to a PVDF membrane, blocked, and stained for β-actin (control) or PTPN2 primary antibody and HRP conjugated secondary antibody. The blot was imaged with the Bio-Rad ChemiDoc and relative PTPN2 expression quantified. PTPN2% Reduction (WB) was calculated using the formula: 1−(adjusted volume intensity of PrimeR+/adjusted volume intensity of PrimeR−)*100. Data are presented as the mean±standard deviation averaged across 4 donors.

### Cytotoxicity Assays

Engineered LG T cells were co-cultured with target cells at varying E:T ratios (1:1, 1:3, 1:9, 1:27, 1:81, and 1:243) for 72 hours at 37° C. Following incubation, cytotoxicity was measured using a luciferase reporter assay. The engineered logic gate T cells comprised the expression module coding for the indicated combination of SLC34A2 priming receptors and TMPRSS4 CARs as shown in Table 25 and the FAS/PTPN2/TGFBR2/TGFBR2 quad shRNA module (SEQ ID NO: 1252 or 972), unless otherwise indicated. Logic gate T cells comprising the indicated SLC34A2 priming receptors and TMPRSS4 CARs, and a control Luc/Luc/Luc/Luc quad shRNA are denoted as “quad Luc shRNA”. Data are presented as the mean±standard deviation of 3 donors.

### CAR Kinetics Assay

For the CAR “ON” kinetics, engineered LG T cells were enriched via magnetic-bead based column enrichment, to obtain a pure transgene-positive fraction using an anti-Whitlow linker Alexa Fluor 647 antibody and anti-Cy5/anti-alexa fluor 647 MicroBeads. Samples were rested for 72 hours. Enriched engineered LG T cells were cultured with either engineered 786-O cell lines that were negative for the priming antigen SLC34A2 or positive for SLC34A2 at an E:T ratio of 1:10. Co-cultures occurred in a 24-well tissue culture plate and standard T cell media supplemented with IL-7 and IL-15, and induced for 96 hours. Every 24 hours, co-cultures were harvested and T-cells were stained for PrimeR and CAR expression using an anti-Whitlow linker Alexa Fluor 647 antibody and an anti-G4S linker PE antibody and analyzed by flow cytometry on an Attune N×T. % CAR induction was calculated using the formula: 100%*(total CAR in SLC34A2 cell line (Q1⁺Q2)/total PrimeR in parental cell line (Q2⁺Q3)). CAR MFI was determined by gating on Live, GFP−, CAR+ cells.

For CAR “OFF” kinetics, enriched engineered LG T cells were cultured with engineered 786-O cell lines that were positive for SLC34A2 at an E:T ratio of 1:10. Cells were cultured in standard T cell media supplemented with IL-7 and IL-15 and were induced for 96 hours. The co-cultured samples were depleted for target cells via magnetic-bead based column enrichment using anti-SLC34A2 PE and anti-PE MicroBeads to obtain a pure population of engineered LG T cells. After removal from priming antigen, the cells were cultured in standard T cell media supplemented with IL-7 and IL-15 for 168 hours with measurements evaluating CAR expression at 24, 48, 72, 96 and 168 hours. Every 24 hours, cultures were harvested and T-cells were stained for PrimeR and CAR expression using an anti-Whitlow linker Alexa Fluor 647 antibody and an anti-G4S linker PE antibody and analyzed by flow cytometry on an Attune N×T.

### Long-Term Repetitive Stimulation Assay (RSA)

For long term cytotoxicity assays, engineered LG T cells were enriched via magnetic-bead based column enrichment, to obtain a pure transgene-positive fraction using an anti-Whitlow linker Alexa Fluor 647 antibody and anti-Cy5/anti-alexa fluor 647 MicroBeads Samples were then left to rest for 48 hours. Engineered LG T cells and RNP controls were co-cultured with 10,000 H1975 target cells engineered to express SLC34A2 and TMPRSS4 at 1:3 Effector: Target (E:T) ratio, at 37° C. Every 3-4 days, half of the cell culture was removed and replated with 10,000 target cells. Co-culture was terminated at 14 days after the initial target cell challenge. Cell cultures were imaged using Incucyte and target cell killing was determined by measuring the fluorescence intensity of GFP signal engineered to express in target cells. Data are presented as mean and SEM of 3 donors.

### Priming Antigen Heterogeneity Cytotoxicity Assay

Engineered LG T cells were co-cultured with 7500 H1975-EFG-SLC34A2/TMPRSS4 and H1975-EFG-TMPRSS4 cells mixed at various ratios to model different levels of priming antigen heterogeneity (ratios used were 100:0, 85:15, 50:50, 5:95, and 0:100 SLC34A2/TMPRSS4: TMPRSS4 cells) as well as different E:T ratios (1:1, 1:3, and 1:9) with the LG T cells. T cells expressing a constitutive CAR with the TPMRSS4 Ab48 binder were also incubated with the heterogeneous target cell populations as a positive control. After 72 hours at 37° C., cytotoxicity was measured using a luciferase reporter assay. Data are presented as the mean±standard deviation of 3 donors.

### Transpriming Assay

Engineered LG T cells were cultured with a mixture of 786-O cells expressing SLC34A2 and 293T cells expressing either WT TMPRSS4 or D290A TMPRSS4 at a 1:1:1 ratio. The expression level of WT and D290A TMPRSS4-positive 293 Ts were matched by flow cytometry to ensure equivalent expression. Supernatant from these cultures were harvested at 72 hrs and levels of IFN-γ were determined using a Lumit Immuno-assay. The % difference in IFNγ produced between the TMPRSS4 cell lines was calculated by using the formula: 100*((IFNγ WT-IFNγ D290A)/(IFNγ D290a)). Data are presented as the average of 3 technical replicates±standard deviation for each of three donor.

### Results

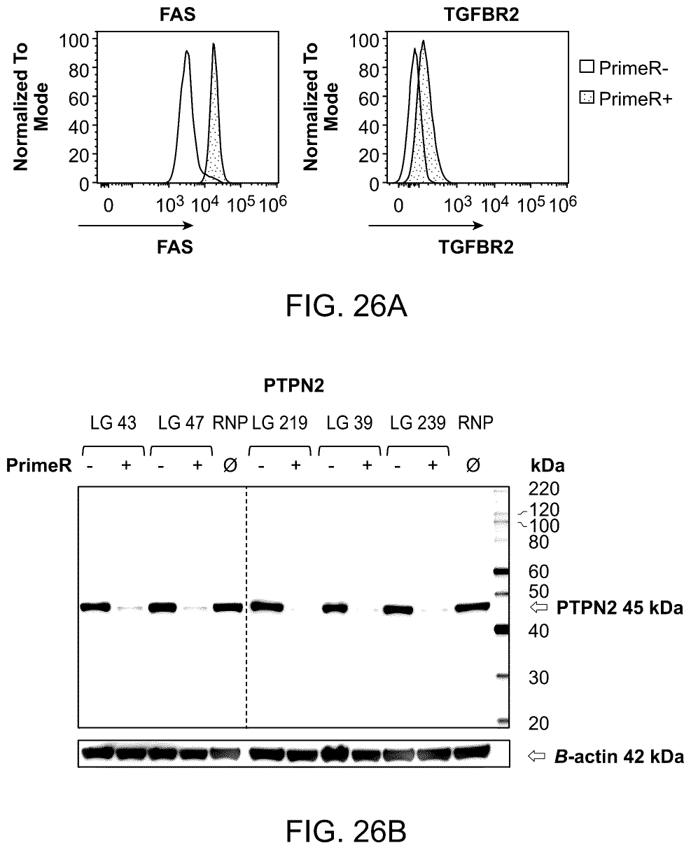

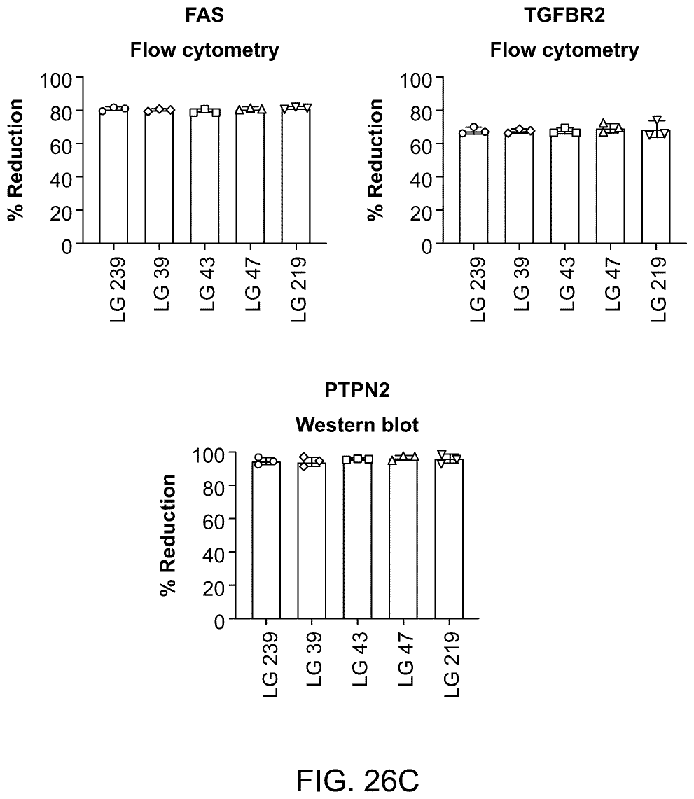

Five logic gates and shRNA module combinations were selected for in vitro characterization (FIG. 24). To characterize the shRNA module of the LG 239, LG 39, LG43, LG47, and LG219 T cell circuits, relative expression of FAS, PTPN2 and TGFBR2 was measured via flow or western blot and % reduction was quantified. Representative histograms of both FAS and TGFBR2 reduction are shown in FIG. 26A. Transgene-negative T cells lacking the Logic Gate and shRNA expression insert (PrimeR−, right peak) showed higher levels of FAS and TGFBR2 expression compared to transgene-positive T cells comprising the Logic Gate and shRNA expression insert (PrimeR+, left peak), indicating that the FAS and TGFBR2 shRNA expressed in the LG T cells reduced FAS and TGFBR2 expression in the T cells. FIG. 26B shows a Western blot image of PTPN2 in transgene-positive and transgene-negative cells from one donor. Transgene-negative samples had a prominent band at 42 kDa corresponding with presence of PTPN2, whereas transgene-positive samples had only a faint band present at the same size, indicating reduction of PTPN2 protein expression in transgene-positive T cells. Unedited cells (RNP) were included as a negative control. FIG. 26C provides quantification of the reduced FAS, TGFBR2 and PTPN2 expression as an average of 3 donors with standard deviation. As shown in FIG. 26C, FAS expression was reduced approximately 80%, TGFBR2 expression was reduced approximately 70% and PTPN2 expression was reduced approximately 95% in the T cells expressing the quad FAS, PTPN2 and 2xTGFBR2 shRNA (SEQ ID NO: 1252). Thus, each of the LG T cell constructs tested showed knockdown of FAS, PTPN2 and TGFBR2 gene expression.

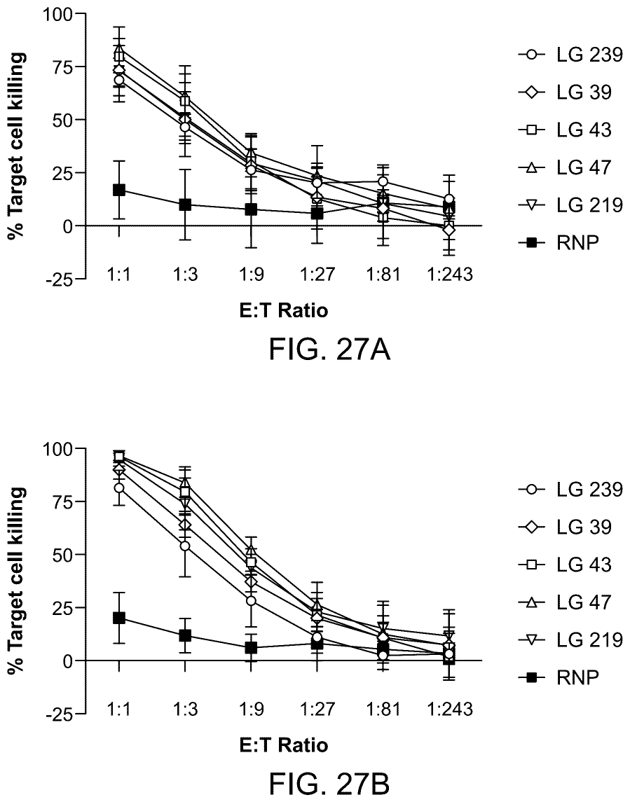

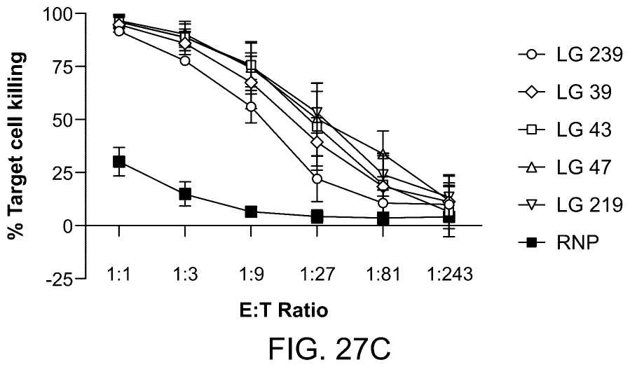

The cytolytic activity of the selected LG T cells were evaluated by co-culturing the T cells with cell lines H1648 or H2347 that endogenously express TMPRSS4 and SLC34A2, and the H1975-SLC34A2/TMPRSS4 cell line engineered to express median levels of both target antigens as observed in primary human tumors as scored by IHC. After 72 hours at 37° C., cytotoxicity was measured using a luciferase reporter assay. Engineered LG T cells targeting SLC34A2 and TMPRSS4 demonstrated potent on-target cytotoxicity against all three dual antigen cell lines (FIG. 27A, FIG. 27B and FIG. 27C). Background cytotoxicity was observed from the negative control RNP-only T cells. Thus, all the selected LG T cells killed indication-specific, dual-antigen cell lines expressing SLC34A2 and TMPRSS4 antigens at and below tumor-relevant levels.

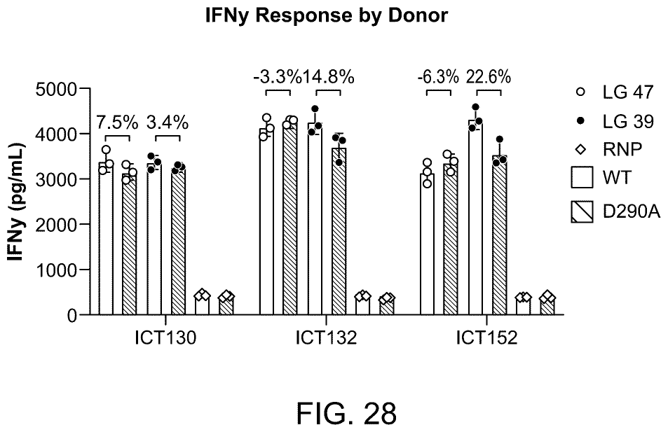

IFNγ production was observed by engineered LG 47 and LG 39 T cells after incubation with both the TMPRSS4 WT and TMPRSS4 D290A cell lines, in T cells engineered from 3 donors (FIG. 28). IFNγ secretion was comparable between cultures with WT or D290A TMPRSS4 for both LG 47 and LG 39 across the three donors tested.

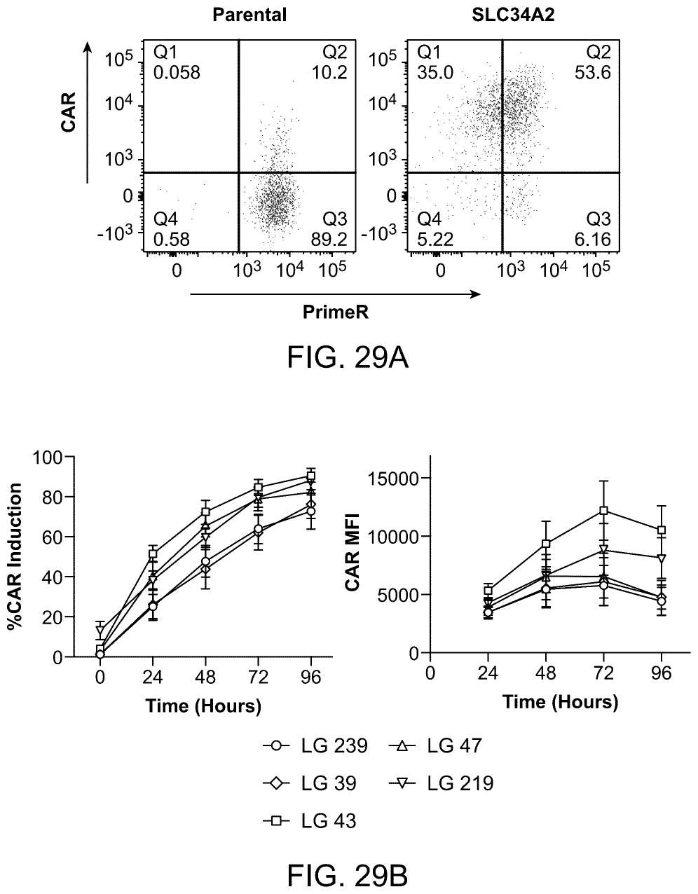

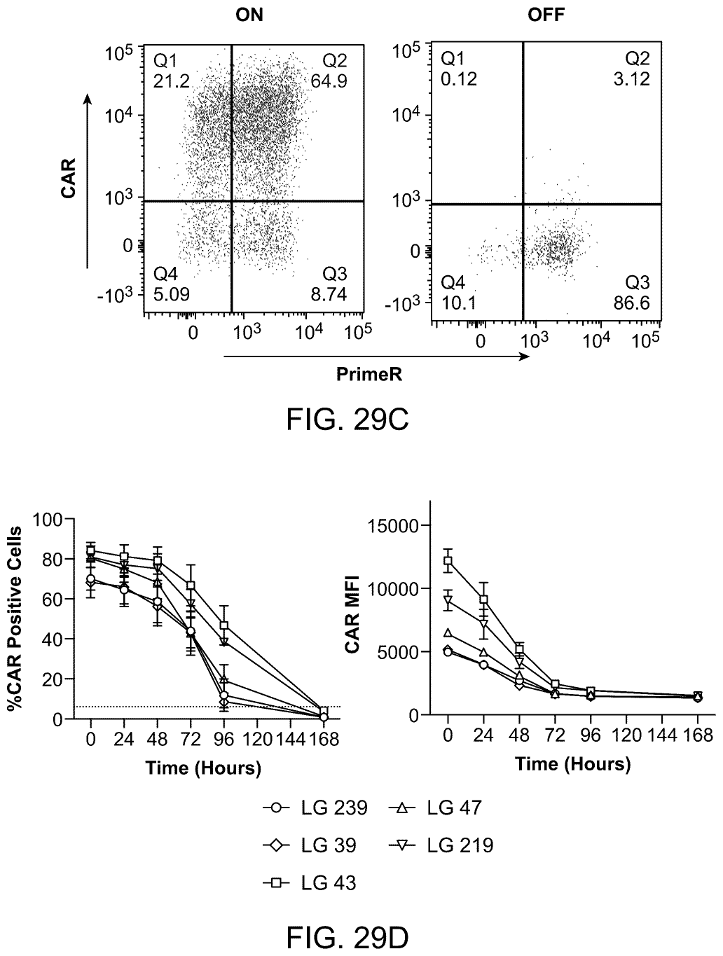

To show that CAR induction in engineered LG T cells occurred in a priming antigen dependent manner (e.g., after contacting a SLC34A2 antigen), an ON/OFF kinetics assay was developed. The CAR kinetics assay demonstrated that all engineered LG T cells cultured with cell lines that expressed the SLC34A2 priming antigen achieved over a 70% CAR induction by 96 hours post onset of induction (FIG. 29B). FIG. 29A shows a representative flow plot of the engineered LG T-cells co-cultured with a parental cell line (left) and an SLC34A2 positive cell line (right). CAR expression was detected only in the context of the SLC34A2-expressing cell line indicating that antigen binding of the priming receptor to its cognate ligand drove CAR expression. FIG. 29C shows representative flow plots of engineered LG T cells removed from priming antigen in an “ON” state (e.g., after incubation with cells expressing SLC34A2) and an “OFF” state (e.g., after incubation with cells that did not express SLC34A2). After removal from the priming antigen, the percent CAR expression (left panel) and CAR mean fluorescent intensity (MFI, right panel) decreased over time, reaching a minimum at around 96 hours (FIG. 29D). Therefore, all selected LG T cells demonstrated SLC34A2-dependent CAR expression induction and rapid loss of CAR expression upon removal of the priming antigen.

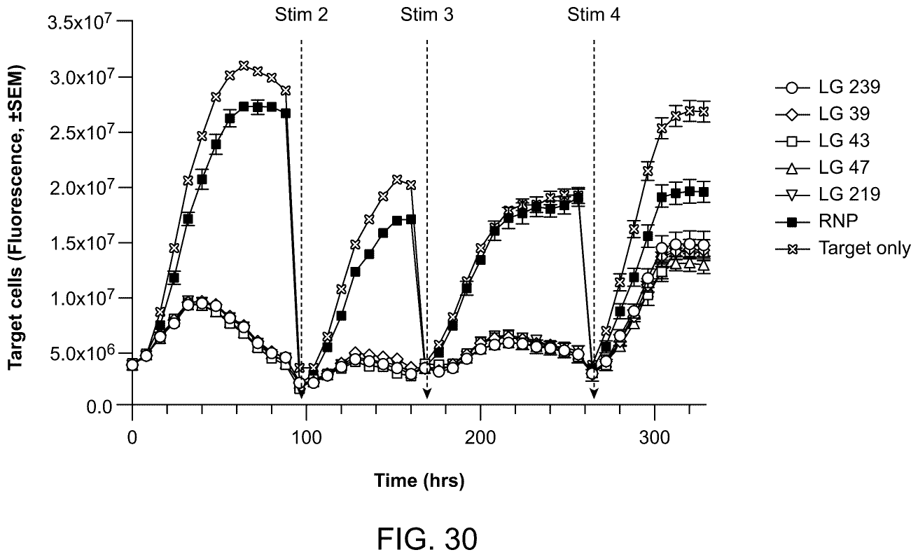

A repetitive stimulation assay (RSA) was also performed, to determine if the efficacy of the engineered LG T cells could be maintained over repeated stimulation with target antigen. The LG T cells maintained long term cytotoxic efficacy against the H1975-SLC34A2/TMPRSS4 target line at 1:3 E:T in the RSA, as compared to the RNP control (FIG. 30).

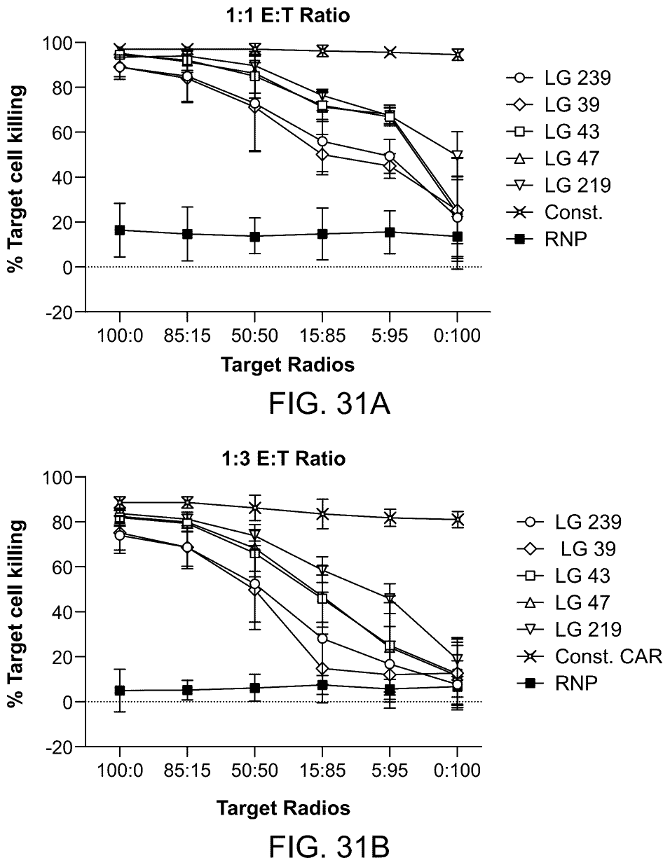

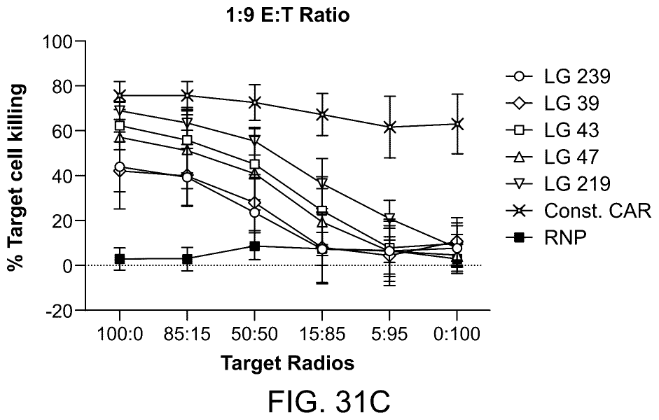

To assess the minimum proportion of prime antigen positive cells necessary to induce target cell killing by the engineered LG T cells, a cytotoxicity assay was developed in which tumor antigen heterogeneity was controlled by mixing target cells H1975-EFG-SLC34A2/TMPRSS4 and H1975-EFG-TMPRSS4 at defined ratios (100:0, 85:15, 50:50, 5:95, and 0:100 SLC34A2/TMPRSS4: TMPRSS4 cells). Heterogenous target cell populations were also incubated with LG T cells as different ratios (E:T ratios (1:1, 1:3, and 1:9). A T cell expressing a constitutive TMPRSS4 CAR comprising an Ab48 extracellular domain was used as a positive control. FIG. 31A shows the % target killing at a 1:1 E:T ratio. FIG. 31B shows the % target killing at a 1:3 E:T ratio. FIG. 31C shows the % target killing at a 1:9 E:T ratio. The constitutive CAR control T cells killed all TMPRSS4 positive target cell conditions equally well regardless of SLC34A2 expression. Only background cytotoxicity was observed from the negative control RNP-only T cells. The priming antigen heterogeneity cytotoxicity assay demonstrated that logic gate T cells were capable of eliminating heterogenous populations of cancer cells that express the priming antigen on only a small minority of cells.

### Example 7: In Vivo Characterization of TMPRSS4 CAR and SLC34A2 Priming Receptor Logic Gate T Cells

### Materials and Methods

### In Vivo Xenograft Tumor Model

To establish an indication-specific lung adenocarcinoma (LUAD) xenograft model, H1975 tumor cells were engineered to express TMPRSS4 and SLC34A2. The H1975-SLC34A2/TMPRSS4 cells were implanted subcutaneously in one flank of NSG MHC I/II double-knockout (NSG-DKO) mice and allowed to grow to palpable tumors of 150 mm³ volume. Engineered LG T-cells or RNP cells were then infused into mice via the tail vein by IV administration (n=7 mice per group) at a dose of 2.5×10⁶ edited cells. Progression in tumor volume was measured using a caliper twice per week. Data are presented as the mean±standard error of the mean (SEM) of 7 mice per test group

### In Vivo Dual Flank Subcutaneous Xenograft Tumor Model

For a dual flank lung adenocarcinoma H1975 subcutaneous xenograft model, H1975 cells expressing TMPRSS4 only (H1975-TMPRSS4) and H1975 cells expressing TMPRSS4 and SLC34A2 (H1975-SLC34A2/TMPRSS4) were subcutaneously injected in contralateral flanks of NSG MHC I/II DKO mice. When the H1975 tumors reached mean tumor volume of 150 mm³, tumor-bearing animals were randomized and injected intravenously with a single dose of 2.5×10⁶ of selected engineered LG (LG 239, LG 39, LG 43, LG47, and LG 219) comprising the quad FAS/PTNP2/2xTGFBR2 shRNA module (SEQ ID NO: 1252), RNP or a constitutive TMPRSS4 CAR with the TPMPRSS4 Ab48 extracellular binding domain T cells. Progression in tumor volume was measured using a caliper twice per week. Data are presented as the mean±standard error of the mean (SEM) of 7 mice per test group.

### Results

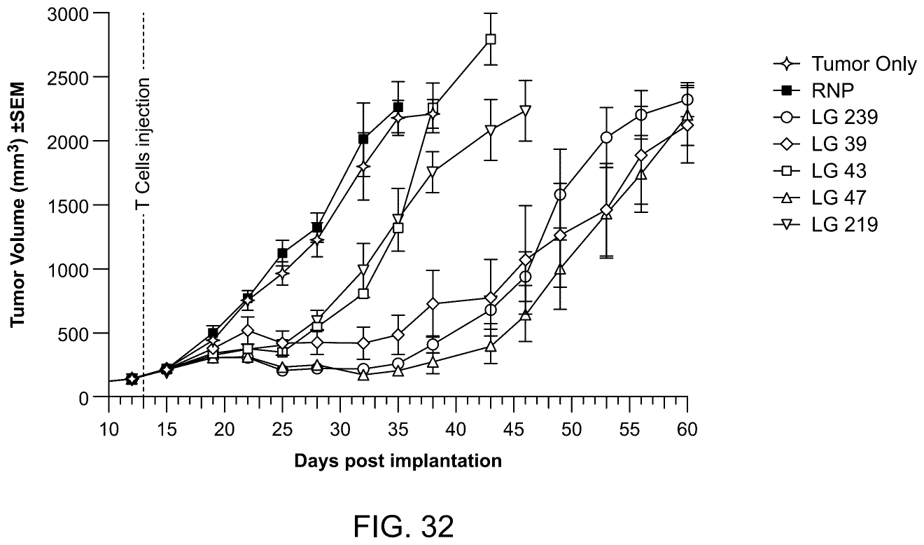

As shown in FIG. 32, all engineered LG T cells demonstrated tumor-growth inhibition at a dose of 2.5×10⁶ cells in the single flank subcutaneous in vivo assay. Three of the five LG T cells (LG 239, LG 39, LG 47) also showed prolonged tumor control (>20 days post T cell injection). By day 10 post T cell injection, mice that received the 2.5×10⁶ dose of the engineered LG T cells achieved anti-tumor effect as defined by reduced tumor volume, compared to mice infused with RNP negative control cells. Thus, engineered LG T cells successfully inhibited growth of H1975-nEFG-SLC43A2-TMPRSS4 non-small cell lung carcinoma tumors in vivo. In addition, LG 47, LG 39, and LG 239 showed the most potent tumor control in this in vivo efficacy study.

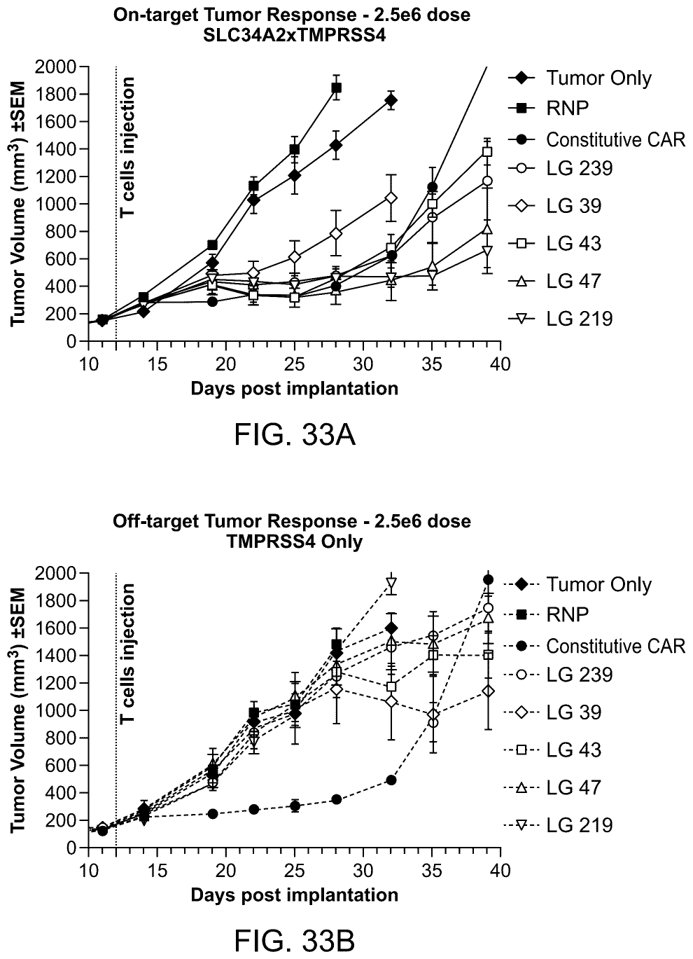

For in vivo specificity, engineered LG T cells, RNP and constitutive CAR T cells were injected into mice bearing two distinct tumors on contralateral flanks. H1975-TMPRSS4 cells were injected into one flank and H1975-TMPRSS4-SLC34A2 cells were injected into the other flank. The LG T cells demonstrated specific tumor growth inhibition against the H1975 SLC34A2-TMPRSS4 expressing tumors (FIG. 33A) but showed minimal impact on the growth of the H1975 TMPRSS4-only expressing tumors (FIG. 33B). LG 47 and LG 239 demonstrated the highest specificity, while LG 47 and LG 39 showed superior on-target response.

T cells expressing the constitutive CAR demonstrated anti-tumor effect on both tumors, confirming the non-specific activity of the CAR as a positive control. In addition, the constitutive CAR T cells initially triggered a tumor response in both on-target and off-target tumors, but lost tumor control over time.

The selected engineered LG T cells demonstrated specific killing of tumors expressing both the priming antigen (SLC34A2) and the cytolytic antigen (TMPRSS4). In addition, the selected LG T cells showed minimal cytolytic effect on the tumors that only expressed TMPRSS4, which were a surrogate for healthy tissues that express TMPRSS4 only. Without wishing to be bound by theory, this demonstrated the functionality and mechanism of action of the sequential AND gate employed in the engineered LG T cells.

### Example 8: In Vitro and In Vivo Characterization of shRNA Modules in TMPRSS4 CAR and SLC34A2 Priming Receptor Logic Gate T Cells

### Materials and Methods

### In Vitro Assays

To characterize the impact of shRNA knockdown of individual gene targets on the LG T cells, engineered LG T cells expressing a control shRNA module comprising a quad luciferase shRNA (Luc/Luc/Luc/Luc, quad luc) as a negative control or shRNA modules individually targeting FAS, PTPN2, or TGFBR2. The new LG T cells were characterized and compared to T cells comprising the full quad FAS/PTPN2/TGFBR2/TGFBR2 shRNA module (SEQ ID NO: 1252) as described in Example 6. In particular, the % knock down of gene expression of FAS, PTPN2, and TGFBR2 was assessed by flow cytometry and cytotoxicity and proliferation of LG 47 T cells in the repeat stimulation assay (RSA) using K562-SLC34A2/TMPRSS4 D290A cells was characterized. In the RSA assay, exogenous human IL-2 cytokine (50 U/mL) was added to the media.

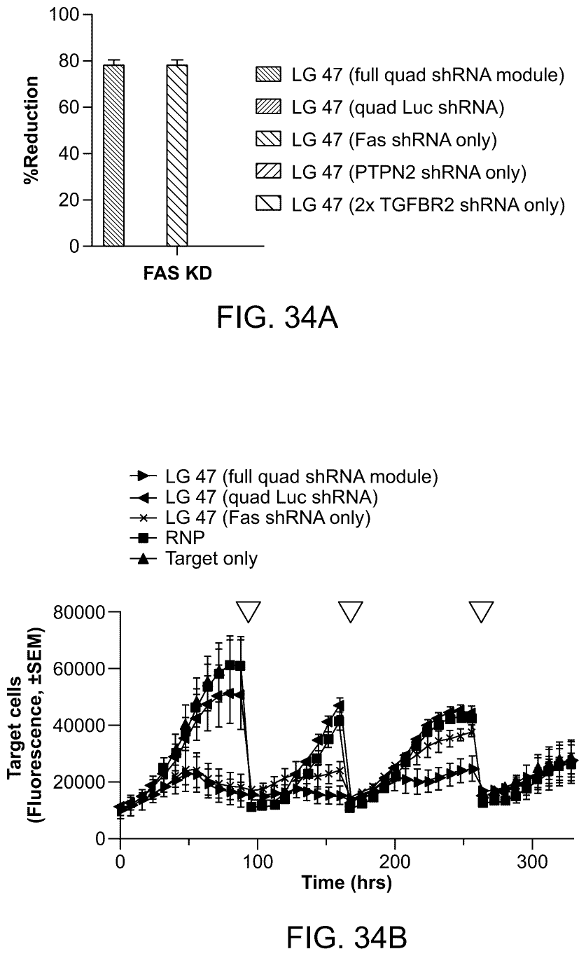

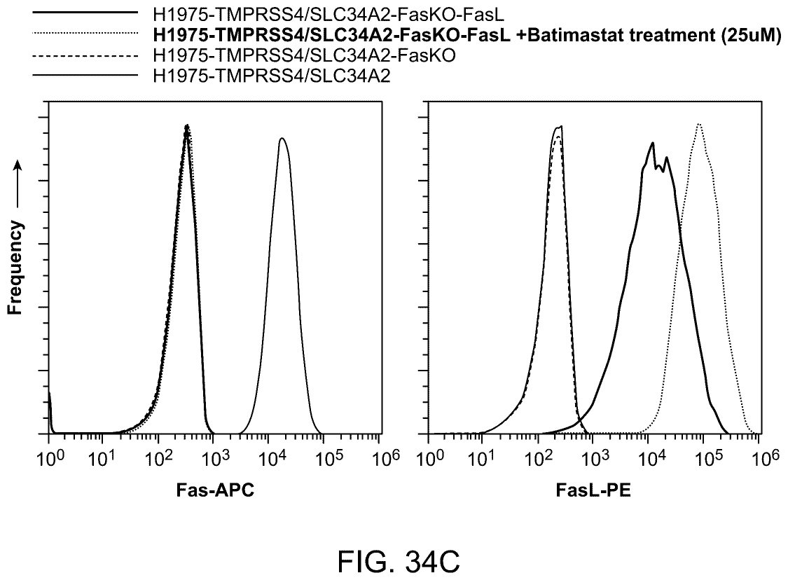

In addition, a H1975-SLC34A2/TMPRSS4 engineered cell line was engineered to knockout FAS expression and overexpress FAS ligand (FASL) (H1975-SLC34A2/TMPRSS4-FasKO-FasL). This cell line was used in the RSA assay as described in Example 6. FIG. 34C shows a histogram of FAS and FASL expression in the double knock out cell line H1975-SLC34A2/TMPRSS4-FasKO-FasL, compared to FAS and FASL expression in cells with only a FAS knock out (H1975-SLC34A2/TMPRSS4-FasKO), the double KO cells treated with bastimastat, and the engineered parental cell line H1975-SLC34A2/TMPRSS4.

pSMAD Assay

Engineered LG T cells comprising the full shRNA module, the 2xTGFBR2 shRNA module, or the quad luc shRNA module were incubated in the presence and absence of 5 ng/ml TGFb for 72 hours. T cells were harvested for quantification of phosphorylated SMAD (pSMAD). pSMAD levels were determined via FACS and quantified as the geometric MFI (gMFI).

### In Vivo Assays

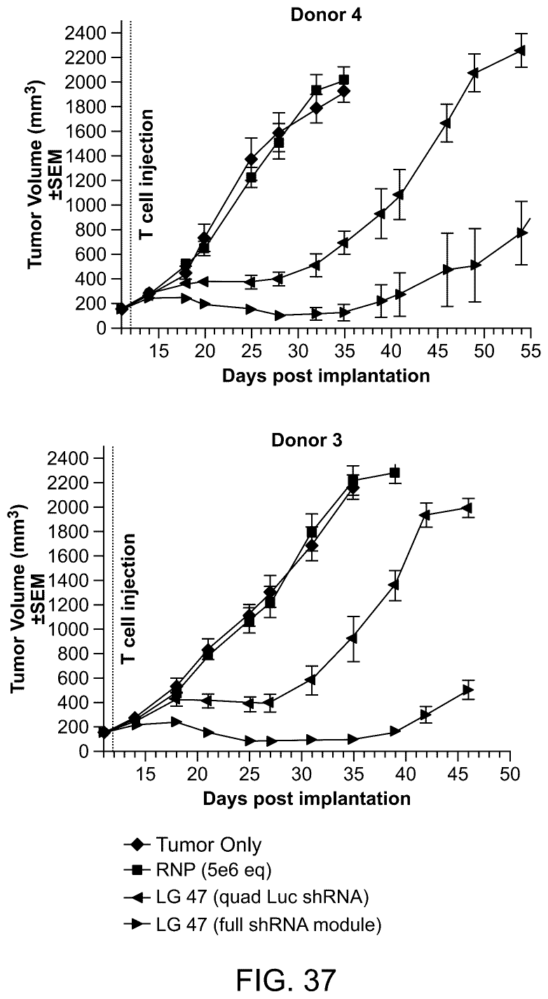

The LG 47 T cells comprising the full shRNA module and the quad luc shRNA module were tested in vivo using the H1975 xenograft tumor model described in Example 7, with the exception of dosing the mice with 5×10⁶ (FIG. 37) LG T cells.

### Results

FIG. 34A shows the % reduction of FAS in engineered LG T cells by flow cytometry. FAS knock down (KD) was observed only in T cells containing a FAS shRNA (e.g., the full shRNA module or the individual FAS shRNA module). FIG. 34B shows the in vitro functional effects of downregulating FAS in T cells incubated with target cells overexpressing FASL and no FAS (H1975-SLC34A2/TMPRSS4-FasKO-FasL). The FAS KD T cells (full shRNA and FAS shRNA) showed improved tumor control as compared to the non-targeting control (quad luc) shRNA T cells over repeated stimulations. Taken together and without wishing to be bound by theory, the data suggest that FAS shRNA knockdown enhanced cytotoxicity in the setting of FASL overexpression.

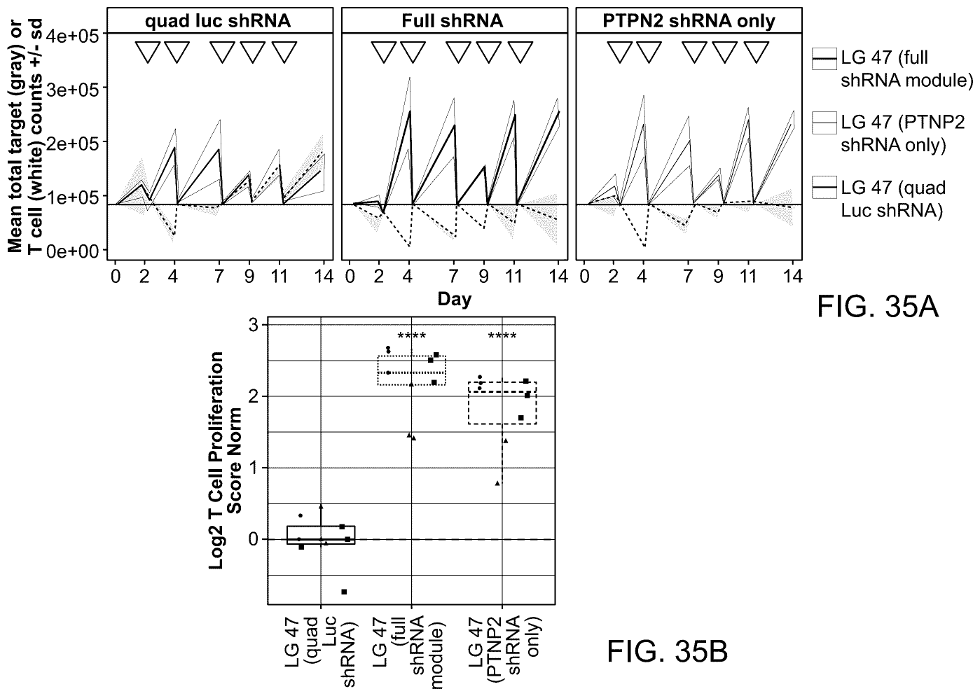

Next, the effects of targeting only the PTPN2 gene were assessed. FIG. 35A shows that shRNA knockdown of PTPN2 enhances T cell cytotoxicity against the target cells and maintained T cell proliferation over time in a repetitive stimulation assay. T cell proliferation of each of the LG T cell lines tested in the RSA is shown in FIG. 35B. The LG T cells expressing the PTPN2 shRNA proliferated while the LG T cells expressing only the control non-targeting quad luc shRNA did not.

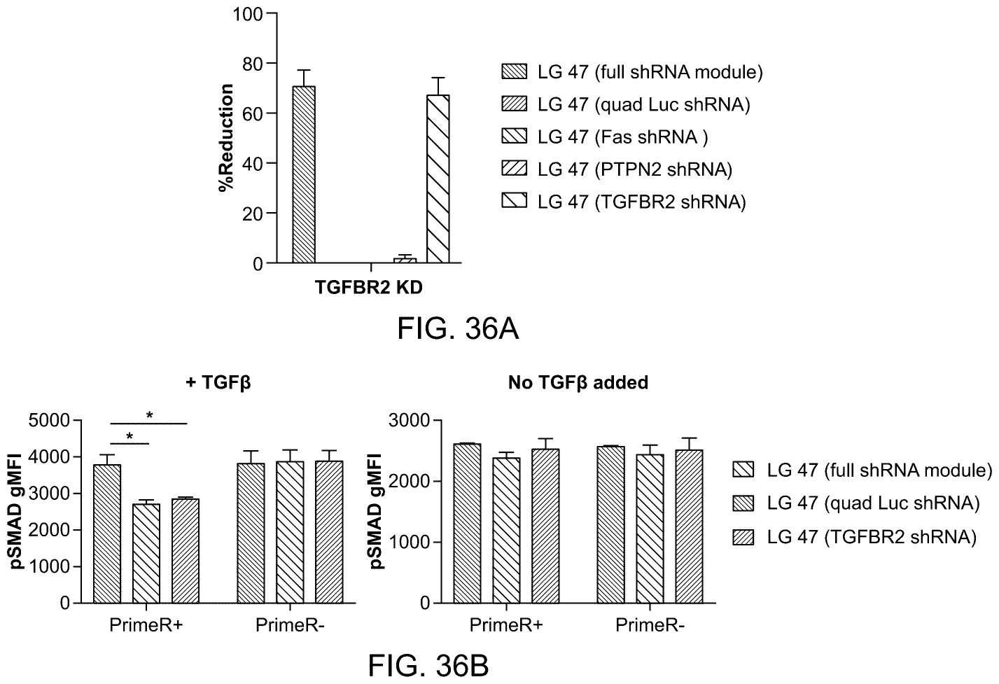

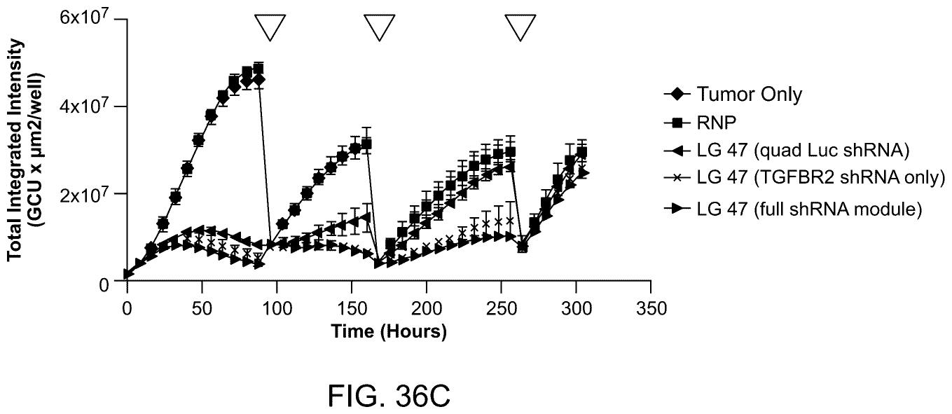

Finally, the effect of downregulation of TGFBR2 on the T cell activity was assessed. FIG. 36A shows % reduction of TGFBR2 by flow cytometry. TGFBR2 knock down (KD) was seen only in the LG T cells comprising a TGFBR2 shRNA (full shRNA module, far left bar and TGFBR2 module, far right bar). FIG. 36B shows that in the presence of exogenous TGFβ, the T cells with a TGFBR2 KD (full shRNA module, middle bar, or TGFBR2 shRNA, right bar) had reduced phosphorylated SMAD as compared to the T cells with the control non-targeting quad luc shRNA (left bar). FIG. 36C shows the in vitro functional effects of TGFBR2 downregulation in the RSA. LG T cells showed effective tumor control after repetitive stimulation when TGFBR2 was knocked down, e.g., in the LG 47 T cells expressing the full shRNA or the TGFBR2 shRNA, but not in the non-targeting quad luc shRNA T cells. Thus, TGFBR2 shRNA knockdown in the LG 47 T cells enhanced T cell cytotoxicity of the LG T cells in the presence of TGFβ-mediated suppression in vitro.

The in vivo H1975 mouse model was also used to assess the effect of the shRNA modules on the function of the LG T cells. The LG 47 T cells comprising the non-targeting control quad luc shRNA had decreased ability to inhibit tumor growth as compared to LG 47 T cells comprising the FAS/PTPN2/TGFBR2/TGFBR2 quad shRNA (FIG. 37). Thus, FAS/PTPN2/TGFBR2 shRNA knockdown in the LG 47 T cells enhanced the T cell cytotoxicity against tumor cells in vivo.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

TABLE 27
Additional Sequences

| Name and SEQ ID | |
|---|---|
| NO | Sequence |
| SLC34A2 peptide | INVTVPSTANCTSPSLCWTDGIQNWTMK |
| fragment | |
| SEQ ID NO: 1149 | |
| SLC34A2 peptide | INVTVPSTANCTSPSLCWTDGIQNWTMKC |
| fragment with C | |
| terminal cysteine | |
| SEQ ID NO: 1150 | |
| KLH | MLSVRLLIVVLALANAENLVRKSVEHLTQEETLDLQAALRELQMDSSSIGFQKIAAAHGA |
| SEQ ID NO: 1151 | PASCVHKDTSIACCIHGMPTFPHWHRAYVVHMERALQTKRRTSGLPYWDWTEPITQLPSL |
| | AADPVYIDSQGGKAHTNYWYRGNIDFLDKKTNRAVDDRLFEKVKPGQHTHLMESVLDALE |
| | QDEFCKFEIQFELAHNAIHYLVGGKHDYSMANLEYTAYDPIFFLHHSNVDRIFAIWQRLQ |
| | ELRNKDPKAMDCAQELLHQKMEPFSWEDNDIPLTNEHSTPADLFDYCELHYDYDTLNLNG |
| | MTPEELKTYLDERSSRARAFASFRLKGFGGSANVFVYVCIPDDNDRNDDHCEKAGDFFVL |
| | GGPSEMKWQFYRPYLFDLSDTVHKMGMKLDGHYTVKAELFSVNGTALPDDLLPHPVVVHH |
| | PEKGFTDPPVKHHQSANLLVRKNINDLTREEVLNLREAFHKFQEDRSVDGYQATAEYHGL |
| | PARCPRPDAKDRYACCVHGMPIFPHWHRLFVTQVEDALVGRGATIGIPYWDWTEPMTHIP |
| | GLAGNKTYVDSHGASHTNPFHSSVIAFEENAPHTKRQIDQRLFKPATFGHHTDLFNQILY |
| | AFEQEDYCDFEVQFEITHNTIHAWTGGSEHFSMSSLHYTAFDPLFYFHHSNVDRLWAVWQ |
| | ALQMRRHKPYRAHCAISLEHMHLKPFAFSSPLNNNEKTHANAMPNKIYDYENVLHYTYED |
| | LTFGGISLENIEKMIHENQQEDRIYAGFLLAGIRTSANVDIFIKTTDSVQHKAGTFAVLG |
| | GSKEMKWGFDRVFKFDITHVLKDLDLTADGDFEVTVDITEVDGTKLASSLIPHASVIREH |
| | ARVKFDKVPRSRLIRKNVDRLSPEEMNELRKALALLKEDKSAGGFQQLGAFHGEPKWCPS |
| | PEASKKFACCVHGMSVFPHWHRLLTVQSENALRRHGYDGALPYWDWTSPLNHLPELADHE |
| | KYVDPEDGVEKHNPWFDGHIDTVDKTTTRSVQNKLFEQPEFGHYTSIAKQVLLALEQDNF |
| | CDFEIQYEIAHNYIHALVGGAQPYGMASLRYTAFDPLFYLHHSNTDRIWAIWQALQKYRG |
| | KPYNVANCAVTSMREPLQPFGLSANINTDHVTKEHSVPFNVFDYKTNFNYEYDTLEFNGL |
| | SISQLNKKLEAIKSQDRFFAGFLLSGFKKSSLVKFNICTDSSNCHPAGEFYLLGDENEMP |
| | WAYDRVFKYDITEKLHDLKLHAEDHFYIDYEVFDLKPASLGKDLFKQPSVIHEPRIGHHE |
| | GEVYQAEVTSANRIRKNIENLSLGELESLRAAFLEIENDGTYESIAKFHGSPGLCQLNGN |
| | PISCCVHGMPTFPHWHRLYVVVVENALLKKGSSVAVPYWDWTKRIEHLPHLISDATYYNS |
| | RQHHYETNPFHHGKITHENEITTRDPKDSLFHSDYFYEQVLYALEQDNFCDFEIQLEILH |
| | NALHSLLGGKGKYSMSNLDYAAFDPVFFLHHATTDRIWAIWQDLQRFRKRPYREANCAIQ |
| | LMHTPLQPFDKSDNNDEATKTHATPHDGFEYQNSFGYAYDNLELNHYSIPQLDHMLQERK |
| | RHDRVFAGFLLHNIGTSADGHVFVCLPTGEHTKDCSHEAGMFSILGGQTEMSFVEDRLYK |
| | LDITKALKKNGVHLQGDFDLEIEITAVNGSHLDSHVIHSPTILFEAGTDSAHTDDGHTEP |
| | VMIRKDITQLDKRQQLSLVKALESMKADHSSDGFQAIASFHALPPLCPSPAASKRFACCV |
| | HGMATFPQWHRLYTVQFQDSLRKHGAVVGLPYWDWTLPRSELPELLTVSTIHDPETGRDI |
| | PNPFIGSKIEFEGENVHTKRDINRDRLFQGSTKTHHNWFIEQALLALEQTNYCDFEVQFE |
| | IMHNGVHTWVGGKEPYGIGHLHYASYDPLFYIHHSQTDRIWAIWQSLQRFRGLSGSEANC |
| | AVNLMKTPLKPFSFGAPYNLNDHTHDFSKPEDTEDYQKFGYIYDTLEFAGWSIRGIDHIV |
| | RNRQEHSRVFAGFLLEGFGTSATVDFQVCRTAGDCEDAGYFTVLGGEKEMPWAFDRLYKY |
| | DITETLDKMNLRHDEIFQIEVTITSYDGTVLDSGLIPTPSIIYDPAHHDISSHHLSLNKV |
| | RHDLSTLSERDIGSLKYALSSLQADTSADGFAAIASFHGLPAKCNDSHNNEVACCIHGMP |
| | TFPHWHRLYTLQFEQALRRHGSSVAVPYWDWTKPIHNIPHLFTDKEYYDVWRNKVMPNPF |
| | ARGYVPSHDTYTVRDVQEGLFHLTSTGEHSALLNQALLALEQHDYCDFAVQFEVMHNTIH |
| | YLVGGPQVYSLSSLHYASYDPIFFIHHSFVDKVWAVWQALQEKRGLPSDRADCAVSLMTQ |
| | NMRPFHYEINHNQFTKKHAVPNDVFKYELLGYRYDNLEIGGMNLHEIEKEIKDKQHHVRV |
| | FAGFLLHGIRTSADVQFQICKTSEDCHHGGQIFVLGGTKEMAWAYNRLFKYDITHALHDA |
| | HITPEDVFHPSEPFFIKVSVTAVNGTVLPASILHAPTIIYEPGLDHHEDHHSSSMAGHGV |
| | RKEINTLTTAEVDNLKDAMRAVMADHGPNGYQAIAAFHGNPPMCPMPDGKNYSCCTHGMA |
| | TFPHWHRLYTKQMEDALTAHGARVGLPYWDGTTAFTALPTFVTDEEDNPFHHGHIDYLGV |
| | DTTRSPRDKLENDPERGSESFFYRQVLLALEQTDFCQFEVQFEITHNAIHSWTGGLTPYG |
| | MSTLEYTTYDPLFWLHHANTDRIWAIWQALQEYRGLPYDHANCEIQAMKRPLRPFSDPIN |
| | HNAFTHSNAKPTDVFEYSRFNFQYDNLRFHGMTIKKLEHELEKQKEEDRTFAAFLLHGIK |
| | KSADVSFDVCNHDGECHFAGTFAILGGEHEMPWSFDRLFRYDITQVLKQMHLEYDSDFTF |
| | HMRIIDTSGKQLPSDLIKMPTVEHSPGGKHHEKHHEDHHEDILVRKNIHSLSHHEAEELR |
| | DALYKLQNDESHGGYEHIAGFHGYPNLCPEKGDEKYPCCVHGMSIFPHWHRLHTIQFERA |
| | LKKHGSHLGIPYWDWTQTISSLPTFFADSGNNNPFFKYHIRSINQDTVRDVNEAIFQQTK |
| | FGEFSSIFYLALQALEEDNYCDFEVQYEILHNEVHALIGGAEKYSMSTLEYSAFDPYFMI |
| | HHASLDKIWIIWQELQKRRVKPAHAGSCAGDIMHVPLHPFNYESVNNDDFTRENSLPNAV |
| | VDSHRFNYKYDNLNLHGHNIEELEEVLRSLRLKSRVFAGFVLSGIRTTAVVKVYIKSGTD |
| | SDDEYAGSFVILGGAKEMPWAYERLYRFDITETVHNLNLTDDHVKFRFDLKKYDHTELDA |
| | SVLPAPIIVRRPNNAVEDIIEIPIGKDVNLPPKVVVKRGTKIMFMSVDEAVTTPMLNLGS |
| | YTAMFKCKVPPFSFHAFELGKMYSVESGDYFMTASTTELCNDNNLRIHVHVDDE |
| SLC34A2 | VEVATHYLEIITQLIVESFHFKNGEDAPDLLKVITKPFTKLIVQLDKKVISQIAMNDEKA |
| recombinant | KNKSLVKIWCKTFTNKTQINVTVPSTANCTSPSLCWTDGIQNWTMKNVTYKENIAKCQHI |
| fusion protein | FVNFHLPDLGSAAAEPRSPTIKPCPPCKCPAPNLEGGPSVFIFPPKIKDVLMISLSPIVT |
| SEQ ID NO: 1152 | CVVVDVSEDDPDVQISWFVNNVEVHTAQTQTHREDYNSTLRVVSALPIQHQDWMSGKAFA |
| | CAVNNKDLPAPIERTISKPKGSVRAPQVYVLPPPEEEMTKKQVTLTCMVTDEMPEDIYVE |
| | WTNNGKTELNYKNTEPVLDSDGSYFMYSKLRVEKKNWVERNSYSCSVVHEGLHNHHTTKS |
| | FSRTPGK |
| SLC34A2 ECL2 | VEVATHYLEIITQLIVESFHFKNGEDAPDLLKVITKPFTKLIVQLDKKVISQIAMNDEKA |
| SEQ ID NO: 1119 | KNKSLVKIWCKTFTNKTQINVTVPSTANCTSPSLCWTDGIQNWTMKNVTYKENIAKCQHI |
| | FVNFHLPDL |
| Murine Fc domain | EPRSPTIKPCPPCKCPAPNLEGGPSVFIFPPKIKDVLMISLSPIVTCVVVDVSEDDPDVQ |
| SEQ ID NO: 1153 | ISWFVNNVEVHTAQTQTHREDYNSTLRVVSALPIQHQDWMSGKAFACAVNNKDLPAPIER |
| | TISKPKGSVRAPQVYVLPPPEEEMTKKQVTLTCMVTDFMPEDIYVEWTNNGKTELNYKNT |
| | EPVLDSDGSYFMYSKLRVEKKNWVERNSYSCSVVHEGLHNHHTTKSFSRTPGK |
| Constant heavy | ASTKGPSVFPLAPSSKSTSGGTAALGCLVKDYFPEPVTVSWNSGALTSGVHTFPAVLQSS |
| chain IgG1 | GLYSLSSVVTVPSSSLGTQTYICNVNHKPSNTKVDKRVEPKSCDKTHTCPPCPAPELLGG |
| SEQ ID NO: 1247 | PSVFLFPPKPKDTLMISRTPEVTCVVVDVSHEDPEVKFNWYVDGVEVHNAKTKPREEQYN |
| | STYRVVSVLTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSKLTVDKSRW |
| | QQGNVFSCSVMHEALHNHYTQKSLSLSPG |
| Constant light | RTVAAPSVFIFPPSDEQLKSGTASVVCLLNNFYPREAKVQWKVDNALQSGNSQESVTEQD |
| kappa chain | SKDSTYSLSSTLTLSKADYEKHKVYACEVTHQGLSSPVTKSENRGEC |
| SEQ ID NO: 1248 | |
| ECL-mFc fusion | VEVATHYLEIITQLIVESFHFKNGEDAPDLLKVITKPFTKLIVQLDKKVISQIAMNDEKA |
| protein | KNKSLVKIWCKTFTNKTQINVTVPSTANCTSPSLCWTDGIQNWTMKNVTYKENIAKCQHI |
| SEQ ID NO: 1249 | FVNFHLPDLGSAAAEPRSPTIKPCPPCKCPAPNLEGGPSVFIFPPKIKDVLMISLSPIVT |
| | CVVVDVSEDDPDVQISWFVNNVEVHTAQTQTHREDYNSTLRVVSALPIQHQDWMSGKAFA |
| | CAVNNKDLPAPIERTISKPKGSVRAPQVYVLPPPEEEMTKKQVTLTCMVTDFMPEDIYVE |
| | WTNNGKTELNYKNTEPVLDSDGSYFMYSKLRVEKKNWVERNSYSCSVVHEGLHNHHTTKS |
| | FSRTPG |

1. A system comprising:
a. a first chimeric polypeptide comprising a priming receptor comprising a first antigen-binding domain that specifically binds to Solute Carrier Family 34 Member 2 (SLC34A2) (SEQ ID NO: 962); and
b. a second chimeric polypeptide comprising a chimeric antigen receptor (CAR) comprising a second antigen-binding domain that specifically binds to Transmembrane protease, serine 4 (TMPRSS4) (SEQ ID NO: 960).

2.-11. (canceled)

12. The system of claim 1, comprising at least one or more nucleic acids comprising a nucleic acid sequence at least 15 nucleotides in length complementary to a portion thereof of:
a. a nucleic acid encoding human Fas Cell Surface Death Receptor (FAS) comprising the sequence set forth in SEQ ID NO: 964; and/or
b. a nucleic encoding human Transforming Growth factor (TGF)-β Receptor 2 (TGFBR2) comprising the sequence set forth in SEQ ID NO: 965; and/or
c. a nucleic acid encoding Phosphatase Non-Receptor Type 2 (PTPN2) comprising the sequence set forth in SEQ ID NO: 966.

13.-70. (canceled)

71. One or more nucleic acids comprising at least one nucleic acid fragment comprising a nucleotide sequence encoding the system of claim 1.

72. The nucleic acid(s) of claim 71, comprising a sequence with at least 80%, 85%, 90%, 91%, 92%, 93%, 94%, 95%, 96%, 97%, 98%, 99%, or 100% identity to a sequence as set forth in SEQ ID NO: 1238; a sequence with at least 80%, 85%, 90%, 91%, 92%, 93%, 94%, 95%, 96%, 97%, 98%, 99%, or 100% identity to a sequence as set forth in SEQ ID NO: 1239; a sequence with at least 80%, 85%, 90%, 91%, 92%, 93%, 94%, 95%, 96%, 97%, 98%, 99%, or 100% identity to a sequence as set forth in SEQ ID NO: 1240; a sequence with at least 80%, 85%, 90%, 91%, 92%, 93%, 94%, 95%, 96%, 97%, 98%, 99%, or 100% identity to a sequence as set forth in SEQ ID NO: 1241; a sequence with at least 80%, 85%, 90%, 91%, 92%, 93%, 94%, 95%, 96%, 97%, 98%, 99%, or 100% identity to a sequence as set forth in SEQ ID NO: 1242; at least 80%, 85%, 90%, 91%, 92%, 93%, 94%, 95%, 96%, 97%, 98%, 99%, or 100% identity to a sequence comprising nucleotides 481-7257 of SEQ ID NO: 1120; a sequence with at least 80%, 85%, 90%, 91%, 92%, 93%, 94%, 95%, 96%, 97%, 98%, 99%, or 100% identity to a sequence comprising nucleotides 481-7239 of SEQ ID NO: 1121; a sequence with at least 80%, 85%, 90%, 91%, 92%, 93%, 94%, 95%, 96%, 97%, 98%, 99%, or 100% identity to a sequence comprising nucleotides 481-7621 of SEQ ID NO: 1122; a sequence with at least 80%, 85%, 90%, 91%, 92%, 93%, 94%, 95%, 96%, 97%, 98%, 99%, or 100% identity to a sequence comprising nucleotides 481-7636 of SEQ ID NO: 1123; or a sequence with at least 80%, 85%, 90%, 91%, 92%, 93%, 94%, 95%, 96%, 97%, 98%, 99%, 100% identity to a sequence comprising nucleotides 481-7621 of SEQ ID NO: 1124; a sequence with at least 80%, 85%, 90%, 91%, 92%, 93%, 94%, 95%, 96%, 97%, 98%, 99%, or 100% identity to a sequence as set forth in SEQ ID NO: 1120; a sequence with at least 80%, 85%, 90%, 91%, 92%, 93%, 94%, 95%, 96%, 97%, 98%, 99%, or 100% identity to a sequence as set forth in SEQ ID NO: 1121; a sequence with at least 80%, 85%, 90%, 91%, 92%, 93%, 94%, 95%, 96%, 97%, 98%, 99%, or 100% identity to a sequence as set forth in SEQ ID NO: 1122; a sequence with at least 80%, 85%, 90%, 91%, 92%, 93%, 94%, 95%, 96%, 97%, 98%, 99%, or 100% identity to a sequence as set forth in SEQ ID NO: 1123; a sequence with at least 80%, 85%, 90%, 91%, 92%, 93%, 94%, 95%, 96%, 97%, 98%, 99%, or 100% identity to a sequence as set forth in SEQ ID NO: 1124; a sequence with at least 80%, 85%, 90%, 91%, 92%, 93%, 94%, 95%, 96%, 97%, 98%, 99%, or 100% identity to a sequence comprising nucleotides 24-7707 of SEQ ID NO: 1120; a sequence with at least 80%, 85%, 90%, 91%, 92%, 93%, 94%, 95%, 96%, 97%, 98%, 99%, or 100% identity to a sequence comprising nucleotides 24-7689 of SEQ ID NO: 1121; a sequence with at least 80%, 85%, 90%, 91%, 92%, 93%, 94%, 95%, 96%, 97%, 98%, 99%, or 100% identity to a sequence comprising nucleotides 24-8071 of SEQ ID NO: 1122; a sequence with at least 80%, 85%, 90%, 91%, 92%, 93%, 94%, 95%, 96%, 97%, 98%, 99%, or 100% identity to a sequence comprising nucleotides 24-8086 of SEQ ID NO: 1123; or a sequence with at least 80%, 85%, 90%, 91%, 92%, 93%, 94%, 95%, 96%, 97%, 98%, 99%, or 100% identity to a sequence comprising nucleotides 24-8071 of SEQ ID NO: 1124.

73. (canceled)

74. One or more nucleic acid(s), wherein the one or more nucleic acid(s) encode:
a. a first chimeric polypeptide comprising a priming receptor comprising a first antigen-binding domain that specifically binds to human Solute Carrier Family 34 Member 2 (SLC34A2);
b. a second chimeric polypeptide comprising a chimeric antigen receptor (CAR) comprising a second antigen-binding domain that specifically binds to human Transmembrane protease, serine 4 (TMPRSS4).

75. The nucleic acid(s) of claim 74, comprising at least one nucleic acid sequence at least 15 nucleotides in length complementary to a portion thereof of:
a. a nucleic acid encoding human Fas Cell Surface Death Receptor (FAS) comprising the sequence set forth in SEQ ID NO: 964; and/or
b. a nucleic acid encoding human Transforming Growth factor (TGF)-Receptor 2 (TGFBR2) comprising the sequence set forth in SEQ ID NO: 965; and/or
c. a nucleic acid encoding Phosphatase Non-Receptor Type 2 (PTPN2) comprising the sequence set forth in SEQ ID NO: 966.

76. (canceled)

77. The nucleic acid(s) of claim 74, wherein the first antigen-binding domain comprises a heavy chain comprising a first variable heavy (VH) chain sequence comprising three heavy chain CDR sequences, CDR-H1, CDR-H2, and CDR-H3, of the VH sequences set forth in SEQ ID NOs: 1001, 1009, or 1015, and a first variable light (VL) chain sequence comprising three light chain CDR sequences, CDR-L1, CDR-L2, and CDR-L3, of the VL sequences set forth in SEQ ID NOs: 1005, 1013, 1125, or 1019, optionally wherein:
a. CDR-H1 comprises the sequence set forth in SEQ ID NO: 1002, CDR-H2 comprises the sequence set forth in SEQ ID NO: 1003, CDR-H3 comprises the sequence set forth in SEQ ID NO: 1004, CDR-L1 comprises the sequence set forth in SEQ ID NO: 1006, CDR-L2 comprises the sequence set forth in SEQ ID NO: 1007, and CDR-L3 comprises the sequence set forth in SEQ ID NO: 1008; or
b. CDR-H1 comprises the sequence set forth in SEQ ID NO: 1010, CDR-H2 comprises the sequence set forth in SEQ ID NO: 1011, CDR-H3 comprises the sequence set forth in SEQ ID NO: 1012, CDR-L1 comprises the sequence set forth in SEQ ID NO: 1006, CDR-L2 comprises the sequence set forth in SEQ ID NO: 1007, and CDR-L3 comprises the sequence set forth in SEQ ID NO: 1014; or
c. CDR-H1 comprises the sequence set forth in SEQ ID NO: 1016, CDR-H2 comprises the sequence set forth in SEQ ID NO: 1017, CDR-H3 comprises the sequence set forth in SEQ ID NO: 1018, CDR-L1 comprises the sequence set forth in SEQ ID NO: 1020, CDR-L2 comprises the sequence set forth in SEQ ID NO: 1021, and CDR-L3 comprises the sequence set forth in SEQ ID NO: 1022.

78.-80. (canceled)

81. The nucleic acid(s) of claim 74, wherein the second antigen-binding domain comprises a second variable heavy (VH) chain sequence comprising three heavy chain CDR sequences, CDR-H1, CDR-H2, and CDR-H3, of the VH sequences set forth in SEQ ID NOs: 319 or 326, and a second variable light (VL) chain sequence comprising three light chain CDR sequences, CDR-L1, CDR-L2, and CDR-L3, of the VL sequence set forth in SEQ ID NOs: 320 or 327, optionally wherein:
a. CDR-H1 comprises the sequence set forth in SEQ ID NO: 321, CDR-H2 comprises the sequence set forth in SEQ ID NO: 322, CDR-H3 comprises the sequence set forth in SEQ ID NO: 323, CDR-L1 comprises the sequence set forth in SEQ ID NO: 324, CDR-L2 comprises the sequence set forth in SEQ ID NO: 325, and CDR-L3 comprises the sequence set forth in SEQ ID NO: 16; and
b. CDR-H1 comprises the sequence set forth in SEQ ID NO: 193, CDR-H2 comprises the sequence set forth in SEQ ID NO: 80, CDR-H3 comprises the sequence set forth in SEQ ID NO: 328, CDR-L1 comprises the sequence set forth in SEQ ID NO: 329, CDR-L2 comprises the sequence set forth in SEQ ID NO: 330, and CDR-L3 comprises the sequence set forth in SEQ ID NO: 331.

82.-95. (canceled)

96. The nucleic acid(s) of claim 74, wherein the at least one or more nucleic acid sequence is encoded in at least one intron region of the nucleic acid.

97.-98. (canceled)

99. The nucleic acid(s) of claim 71, wherein the nucleic acid comprises:
(i) an inducible promoter operably linked to the nucleotide sequence encoding the CAR, wherein the inducible promoter drives the inducible expression of the CAR;
(ii) a constitutive promoter operably linked to the nucleotide sequence encoding the priming receptor, wherein the constitutive promoter drives constitutive expression of the priming receptor; or
(iii) an inducible promoter element operably linked to the nucleotide sequence encoding the CAR and a constitutive promoter operably linked to the nucleotide sequence encoding the priming receptor.

100.-112. (canceled)

113. The nucleic acid(s) of claim 71, wherein the nucleic acid comprises a 5′ homology directed repair arm and a 3′ homology directed repair arm, both of which are complementary to an insertion site in a host cell chromosome.

114.-118. (canceled)

119. A vector comprising the nucleic acid of claim 71.

120.-126. (canceled)

127. An isolated cell comprising
at least one nucleic acid of claim 71.

128. The isolated cell of claim 127, wherein the cell is an immune cell.

129. (canceled)

130. The isolated cell of claim 128, wherein the immune cell is a primary human immune cell.

131. The isolated cell of claim 130, wherein the primary immune cell is a natural killer (NK) cell, a T cell, a CD8+ T cell, a CD4+ T cell, a primary T cell, or a T cell progenitor.

132.-133. (canceled)

134. A population of isolated cells comprising a plurality of cells of claim 127.

135. A pharmaceutical composition comprising the isolated cells of claim 127, and a pharmaceutically acceptable excipient.

136. A pharmaceutical composition comprising the nucleic acid of claim 71, and a pharmaceutically acceptable excipient.

137. A method of editing a cell, comprising inserting the nucleic acid of claim 71 into a genome of the cell.

138.-139. (canceled)

140. A method of killing or inhibiting a target cell in a subject comprising administering the cells of claim 127 to the subject, wherein the cell kills the target cell and/or triggers cytolysis of the target cell.

141.-143. (canceled)

144. A method of treating a disease in a human subject comprising administering the cells of claim 127 to the subject.

145. The method of claim 144, wherein the disease is cancer.

146.-147.

148. A method of treating a disease in a subject comprising:
a. determining or having determined the presence of human SLC34A2-positive (SLC34A2+) cells in a cancer sample obtained from the subject; and/or
b. determining or having determined the presence of human TMPRSS4-positive (TMPRSS4+) cells in a cancer sample obtained from the subject; and
c. administering the cell or immune cell of claim 127 to the subject.

149-151. (canceled)

152. A method of modulating the activity of a cell or immune cell comprising:
a. obtaining a cell or immune cell comprising
the nucleic acid of claim 71;
and
b. contacting the cell or immune cell with a target cell expressing SLC34A2 and TMPRSS4, wherein binding of the priming receptor to SLC34A2 on the target cell induces activation of the priming receptor and expression of the chimeric antigen receptor and wherein binding of the chimeric antigen receptor to TMPRSS4 on the target cell modulates the activity of the immune cell.

153.-159. (canceled)

160. A method of inducing expression of a chimeric antigen receptor with a priming receptor in a cell comprising:
a. obtaining a cell or immune cell comprising
the nucleic acid of claim 71;
and
b. contacting the cell or immune cell with a cell expressing SLC34A2, wherein binding of the priming receptor to SLC34A2 on the cell induces activation of the priming receptor and expression of the chimeric antigen receptor.

161.-167. (canceled)

168. An isolated human cell expressing a priming receptor comprising an antigen-binding domain that specifically binds to human SLC34A2 and a CAR comprising an antigen-binding domain that specifically binds to human TMPPRSS4, wherein binding of the priming receptor to SLC34A2 on the surface of a target cell and binding of the CAR to TMPRSS4 on the surface of a target cell induces lysis of the cell expressing TMPRSS4.

169. (canceled)

170. One or more nucleic acids comprising a nucleic acid sequence encoding a first cell surface receptor that specifically binds to human SLC34A2 and a nucleic acid sequence encoding a second cell surface receptor that specifically binds to human TMPRSS4, wherein binding of the first and second cell surface receptors to SLC34A2 and TMPRSS4 on the surface of one or more human cells, respectively, induces lysis of a human cell with TMPRSS4 on the surface.

171.-181. (canceled)

182. A cell comprising a nucleotide sequence comprising SEQ ID NO: 1238, 1239, 1240, 1241, or 1242 or a nucleotide sequence that differs therefrom in at most 50 nucleotides, wherein the differences are silent substitutions, additions or deletions.

183. A cell comprising a nucleotide sequence comprising SEQ ID NO: 1120, 1121, 1122, 1123, or 1124 or a nucleotide sequence that differs therefrom in at most 50 nucleotides, wherein the differences are silent substitutions, additions or deletions.

184.-188. (canceled)
